(19) United States
(12) Patent Application Publication
(10) Pub. No.: US 2025/0152717 A1
(43) Pub. Date: May 15, 2025

(54) BINDING PROTEINS AND ENGINEERED CELLS SPECIFIC FOR NEOANTIGENS AND USES THEREOF

(71) Applicants: Affini-T Therapeutics, Inc., Watertown, MA (US); Fred Hutchinson Cancer Center, Seattle, WA (US)

(72) Inventors: Philip GREENBERG, Mercer Island, WA (US); Tijana MARTINOV, Seattle, WA (US); Rachel PERRET, Wellington (NZ); Gary SHAPIRO, Arlington, MA (US); Xingyue HE, Natick, MA (US); Allison DRAIN, Watertown, MA (US); Hongjing QU, Watertown, MA (US); Michele HOFFMANN, Medfield, MA (US); Jinsheng LIANG, Shrewsbury, MA (US); Thomas SCHMITT, Seattle, WA (US)

(73) Assignees: Affini-T Therapeutics, Inc., Watertown, MA (US); Fred Hutchinson Cancer Center, Seattle, WA (US)

(21) Appl. No.: 18/955,703

(22) Filed: Nov. 21, 2024

Related U.S. Application Data
(63) Continuation of application No. PCT/US2023/023150, filed on May 22, 2023.
(60) Provisional application No. 63/501,973, filed on May 12, 2023.
(60) Provisional application No. 63/380,527, filed on Oct. 21, 2022.
(60) Provisional application No. 63/344,965, filed on May 23, 2022.

(51) Int. Cl. A61K 40/42; A61K 40/11; A61K 40/32; A61P 35/00; C07K 14/725; C07K 16/40

(52) U.S. Cl. CPC A61K 40/4253; A61K 40/11; A61K 40/32; A61P 35/00; C07K 14/7051; C07K 16/40

(57) ABSTRACT

The present disclosure provides compositions and methods for targeting a neoantigen to, for example, treat or prevent cancer. Disclosed embodiments include binding proteins, such as T cell receptors bind to a neoantigen:HLA complex. Disclosed binding proteins are highly sensitive to antigen, capable of inducing activation of host T cells at low concentrations of peptide antigen. In certain embodiments, binding proteins of the present disclosure are non-alloreactive against, are substantially non-alloreactive against, and/or have a low risk of alloreactivity against (i) amino acid sequences from the human proteome and/or (ii) against human HLA alleles. Polynucleotides encoding such binding protein can introduced into a host cell, such as a T cell, and the cell can be used in immunotherapy for treating various cancers.

### CROSS-REFERENCE TO RELATED APPLICATIONS

This application is a continuation under 35 U.S.C. § 111(a) of PCT International Patent Application No. PCT/US2023/023150, filed May 22, 2023, designating the United States and published in English, which claims priority to and the benefit of U.S. Provisional Application No. 63/344,965, filed on May 23, 2022; 63/380,527, filed on Oct. 21, 2022; and 63/501,973, filed on May 12, 2023, the entire contents of which are incorporated by reference herein.

### SEQUENCE LISTING

The present application contains a Sequence Listing which has been submitted electronically in XML format. The entire contents of the electronic XML Sequence Listing (Date of creation: Jan. 29, 2025; Size: 1,112,948 bytes; Name: 218378-040205US_SL.xml) are incorporated by reference herein.

### BACKGROUND

T cell-based immunotherapies began to be developed when tumor-reactive T cells were found among a population of tumor-infiltrating lymphocytes (TILs). One strategy, known as adoptive T cell transfer, in some contexts involves the isolation of tumor infiltrating lymphocytes pre-selected for tumor-reactivity, clonal expansion of the tumor-reactive T cells induced by anti-CD3 and anti-CD28 antibodies in the presence of IL-2, and infusing the expanded cell population back to the tumor-bearing patient. Isolation of tumor-reactive T cell clones led to the development of another immunotherapeutic approach—the generation of recombinant T cell receptors (TCRs) specific for particular antigens, which may be introduced into T cells, e.g., using a vector delivery system, to confer specificity for a desired target such as a tumor-associated peptide presented by a major histocompatibility complex (MHC) molecule expressed on a tumor cell (known as human leukocyte antigen (HLA) molecule in humans).

### SUMMARY OF THE INVENTION

In some aspects, the present disclosure provides for: a polynucleotide comprising a nucleic acid sequence encoding: (a) a binding protein, wherein the binding protein comprises: (i) a T cell receptor (TCR) or a functional derivative thereof; or (ii) a chimeric antigen receptor (CAR) or a functional derivative thereof; and (b) a fusion protein, wherein the fusion protein comprises: (i) an extracellular component comprising a CD95 ligand (FasL) binding domain that comprises a CD95 (Fas) ectodomain or a functional fragment thereof; and (ii) an intracellular component comprising a CD137 (4-1BB) intracellular signaling domain, wherein the nucleic acid sequence encoding the binding protein is positioned upstream of the nucleic acid sequence encoding the fusion polypeptide. In some embodiments, the polynucleotide further comprises a nucleic acid sequence encoding: (c) a CD8 co-receptor α or β chain or a portion or variant thereof, wherein the sequence encoding the binding protein is positioned upstream of the sequence encoding the extracellular portion of a CD8 co-receptor α or β chain or the portion or variant thereof. In some embodiments, the polynucleotide further comprises a nucleic acid sequence encoding: (c) a CD8 co-receptor α and β chain or portions or variants thereof, wherein the sequence encoding the binding protein is positioned upstream of the sequence encoding the extracellular portion of the CD8 co-receptor α and β chains or the portions or variants thereof. In some embodiments, the nucleic acid sequence encoding the fusion protein further encodes: a hydrophobic component between the extracellular and intracellular components of the fusion protein. In some embodiments, the binding protein comprises a binding domain that binds to a peptide:HLA complex, wherein the complex comprises a neoantigen peptide and an HLA protein. In some embodiments, the binding protein comprises a single-chain TCR (scTCR) or a single-chain T cell receptor variable fragment (scTv). In some embodiments, the binding protein comprises a TCR α chain variable (Vα) domain or a TCR β chain variable (Vβ) domain. In some embodiments, the binding protein comprises a TCR α chain variable (Vα) domain and a TCR β chain variable (Vβ) domain. In some embodiments, the CD95 (Fas) ligand binding domain is a Fas ectodomain or a functional fragment thereof. In some embodiments, the intracellular component is a CD137 (4-1BB) transmembrane domain or a functional fragment thereof. In some embodiments, the CD95 (Fas) ectodomain or a functional fragment thereof comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 81, or the CD137 (4-1BB) intracellular signaling domain comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:82. In some embodiments, the fusion protein comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 80. In some embodiments, the nucleic acid sequence encoding the fusion protein comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 83. In some embodiments, the CD95 (Fas) ectodomain or functional fragment thereof comprises at least one of residues R68, F97, K100, R102, R103, L106, F133, H142 of SEQ ID NO: 81. In some embodiments, the CD137 (4-1BB) intracellular signaling domain or a portion or variant thereof comprises of the amino acid sequence of SEQ ID NO:82. In some embodiments, the CD8 co-receptor α or β chain or a portion or variant thereof comprises the amino acid sequence of SEQ ID NO:65 or the amino acid sequence of SEQ ID NO:66. In some embodiments, the neoantigen peptide is a KRAS, HRAS, NRAS, p53, or PIK3CA mutant peptide. In some embodiments, the KRAS mutant peptide comprises x-V-G-A-x-G-x-x-K, wherein x denotes any amino acid. In some embodiments, the KRAS mutant peptide is a KRAS G12V mutant peptide. In some embodiments, the KRAS G12V mutant peptide comprises the amino acid sequence VVVGAVGVGK (SEQ ID NO:2) or VVGAVGVGK (SEQ ID NO:3). In some embodiments, the HLA protein is encoded by an HLA-A*11 or HLA-A*11:01 allele. In some embodiments, the polynucleotide further comprises a nucleic acid sequence encoding a self-cleaving peptide between the nucleic acid sequence encoding the TCR receptor variable α (Vα) region and the nucleic acid sequence encoding the TCR receptor variable β (Vβ) region. In some embodiments, the polynucleotide further comprises a nucleic acid sequence encoding a self-cleaving peptide disposed between (a) and (b) or, where (c) is present, (b) and (c). In some embodiments, the polynucleotide further comprises a nucleic acid sequence encoding a self-cleaving peptide between the sequence encoding the CD8 co-receptor α chain and the sequence encoding the CD8 co-receptor β chain. In some embodiments, the polynucleotide further comprises a nucleic acid sequence that encodes a self-cleaving peptide that is disposed between the nucleic acid sequence encoding a binding protein and the nucleic acid sequence encoding a polypeptide comprising an extracellular portion of a CD8 co-receptor α chain; and/or the nucleic acid sequence encoding a binding protein and the nucleic acid sequence encoding a polypeptide comprising an extracellular portion of a CD8 co-receptor β chain. In some embodiments, the polynucleotide further comprises, operably linked in-frame:(i) (pnBP)-(pnSCP₁)-(pnCD8α)-(pnSCP₂)-(pnCD8β)-(pnFP); (ii) (pnBP)-(pnSCP₁)-(pnCD8β)-(pnSCP₂)-(pnCD8α)-(pnFP); (iii)(pnBP)-(pnSCP₁)-(pnFP)-(pnSCP₁)-(pnCD8α)-(pnSCP₂)-(pnCD8β); or (iv) (pnBP)-(pnSCP₁)-(pnFP)-(pnSCP₁)-(pnCD8β)-(pnSCP₂)-(pnCD8α); wherein pnCD8α is the nucleic acid sequence encoding a polypeptide that comprises an extracellular portion of a CD8 co-receptor α chain, wherein pnCD8β is the nucleic acid sequence encoding a polypeptide that comprises an extracellular portion of a CD8 co-receptor α chain, wherein pnBP is the nucleic acid sequence encoding a binding protein, wherein pnFP is the nucleic acid sequence encoding a fusion protein, and wherein pnSCP₁ and pnSCP₂ are each independently a polynucleotide encoding a self-cleaving peptide, wherein the polynucleotides and/or the encoded self-cleaving peptides are optionally the same or different. In some embodiments, the self-cleaving peptide is a P2A, T2A, E2A, or a furin peptide. In some embodiments, the P2A, T2A, or E2A peptide comprises the amino acid sequence of SEQ ID NO:74, 75, or 76, respectively. In some embodiments, the furin peptide comprises the amino acid sequence RAKR. In some embodiments, the binding protein and fusion protein are encoded in a single construct or continuous genomic segment. In some embodiments, the binding protein, fusion protein, and CD8α or CD8β or both are encoded in a single construct or continuous genomic segment. In some embodiments, the binding protein and fusion protein are encoded in a single open reading frame. In some embodiments, binding protein and fusion protein are operably linked to a single promoter. In some embodiments, binding protein and fusion protein are operably linked to different promoters.

In some aspects, the present disclosure provides for a vector comprising any of the polynucleotides described herein. In some embodiments, the vector is a viral vector. In some embodiments, the viral vector is a lentiviral vector or a γ-retroviral vector.

In some aspects, the present disclosure provides for a host cell comprising any of the polynucleotides or any of the vectors described herein. In some embodiments, the host cell does not replicate for more than 5, 6, 7 8, 9, 10, 12, 14, 16, 18, 24, 36, or 48 hours in the absence of exogenous cytokines. In some embodiments, the host cell is a hematopoietic progenitor cell or human immune cell. In some embodiments, the host cell is a human immune cell and the human immune cell comprises a T cell, a NK cell, a NK-T cell, a dendritic cell, a macrophage, a monocyte, or any combination thereof. In some embodiments, the human immune cell comprises a T cell, the T cell comprises a CD4⁺ T cell, a CD8⁺ T cell, a CD4⁻ CD8⁻ double negative T cell, a γδ T cell, a naïve T cell, a central memory T cell, a stem cell memory T cell, an effector memory T cell, or any combination thereof.

In some aspects, the present disclosure provides for a method for treating a disease or disorder associated with a KRAS G12V mutation or a NRAS G12V mutation or a HRAS G12V mutation in a subject, the method comprising administering to the subject an effective amount of any of the host cells described herein. In some embodiments, the disease or disorder comprises a cancer. In some embodiments, the cancer is a solid cancer or a hematological malignancy. In some embodiments, the cancer is a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Myelomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatosis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma. In some embodiments, the effective amount of the host cell is administered to the subject parenterally or intravenously. In some embodiments, the effective amount comprises about 10⁴ cells/kg to about 10¹¹ cells/kg. In some embodiments, the effective amount comprises CD4⁺ T cells and CD8⁺ T cells. In some embodiments, the effective amount comprises substantial amounts of CD4⁺ T cells and CD8⁺ T cells. In some embodiments, the method further comprises administering a cytokine to the subject. In some embodiments, the cytokine comprises IL-2, IL-15, or IL-21. In some embodiments, the subject has received or is receiving an immune checkpoint inhibitor and/or an agonist of a stimulatory immune checkpoint agent. In some embodiments, the subject has received myeloablation therapy. In some embodiments, the cancer is reduced by at least 50%, at least 60%, at least 70%, at least 80%, or at least 90% in a period following administering the effective amount of the host cells. In some embodiments, the period comprises fewer than or equal to 120 days, fewer than or equal to 60 days, fewer than or equal to 50 days, fewer than or equal to 40 days, fewer than or equal to 30 days, or fewer than or equal to 20 days. In some embodiments, the method further comprises administering at least a second dose.

In some aspects, the present disclosure provides for a method of eliciting an immune reaction against a cell expressing a neoantigen, the method comprising contacting the cell with a cell comprising any of the polynucleotides or vectors described herein.

In some aspects, the present disclosure provides for a method of eliciting an immune reaction against a cell expressing a neoantigen, the method comprising contacting the cell with any of the host cells described herein. In some embodiments, the cell is a cancer cell. In some embodiments, the cancer cell is pancreatic cancer cell, a lung cancer cell, or a colorectal cancer cell. In some embodiments, the pancreatic cancer cell is a pancreatic ductal adenocarcinoma cell. In some embodiments, the lung cancer cell is a non-small cell lung cancer cell.

In some aspects, the present disclosure provides for a method of genetically engineering an immune cell, the method comprising contacting the cell with a polynucleotide comprising a nucleic acid sequence encoding a T cell receptor (TCR) or functional fragment or variant thereof, a CD8α and/or a CD8β co-receptor or functional fragment or variant thereof, and a fusion protein comprising a CD95 (Fas) ectodomain or a functional fragment thereof and an intracellular component comprising a CD137 (4-1BB) intracellular signaling domain, and expanding the immune cell. In some embodiments, the polynucleotide is any of the polynucleotides or any of the vectors described herein.

In some aspects, the present disclosure provides for a host cell comprising: (a) a fusion protein, wherein the fusion protein comprises: (i) an extracellular component comprising a CD95 ligand (FasL) binding domain that comprises a CD95 (Fas) ectodomain or a functional fragment thereof; and (ii) an intracellular component comprising a CD137 (4-1BB) intracellular signaling domain, wherein the nucleic acid sequence encoding the binding protein is positioned upstream of the nucleic acid sequence encoding the fusion polypeptide; and (b) an exogenous CD8 co-receptor α or β chain or a portion or variant thereof. In some embodiments, the exogenous CD8 co-receptor α or β chain or a portion or variant thereof is expressed from a locus other than a native locus of a CD8 co-receptor α or β chain. In some embodiments, the host cell comprises an mRNA encoding the exogenous CD8 co-receptor α or β chain or a portion or variant thereof comprising a non-native 3′ or 5′ untranslated region (UTR). In some cases, a sequence encoding exogenous CD8 co-receptor α or β chain or a portion or variant thereof is on a same mRNA with a sequence encoding the fusion polypeptide. In some embodiments, the non-native 3′ or 5′ UTR is a viral UTR, an adenoviral UTR, or a lentiviral UTR. In some embodiments, the host cell comprises a native TCR. exogenous CD8 co-receptor α or β chain or a portion or variant thereof the fusion protein further encodes a hydrophobic component between the extracellular and intracellular components of the fusion protein. In some embodiments, the CD95 (Fas) ligand binding domain is a Fas ectodomain or a functional fragment thereof. In some embodiments, the intracellular component is a CD137 (4-1BB) transmembrane domain or a functional fragment thereof. In some embodiments, the CD95 (Fas) ectodomain or a functional fragment thereof comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 81, or the CD137 (4-1BB) intracellular signaling domain comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:82. In some embodiments, the fusion protein comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 80. In some embodiments, the CD95 (Fas) ectodomain or functional fragment thereof comprises at least one of residues R68, F97, K100, R102, R103, L106, F133, H142 of SEQ ID NO: 81. In some embodiments, the CD137 (4-1BB) intracellular signaling domain or a portion or variant thereof comprises of the amino acid sequence of SEQ ID NO:82. In some embodiments, the CD8 co-receptor α or β chain or a portion or variant thereof comprises the amino acid sequence of SEQ ID NO:65 or the amino acid sequence of SEQ ID NO:66. In some embodiments, the host cell further comprises a binding protein comprising an exogenous TCR. In some embodiments, the binding protein comprises a binding domain that binds to a peptide:HLA complex, wherein the complex comprises a neoantigen peptide and an HLA protein. In some embodiments, the neoantigen peptide is a KRAS, HRAS, NRAS, p53, or PIK3CA mutant peptide In some embodiments, the KRAS mutant peptide comprises x-V-G-A-x-G-x-x-K, wherein x denotes any amino acid. In some embodiments, the neoantigen peptide is a KRAS mutant peptide, wherein the KRAS mutant peptide is a KRAS G12V mutant peptide. In some embodiments, the KRAS G12V mutant peptide comprises the amino acid sequence VVVGAVGVGK (SEQ ID NO:2) or VVGAVGVGK (SEQ ID NO:3). In some embodiments, the HLA protein is encoded by an HLA-A*11 or HLA-A*11:01 allele. In some embodiments, the fusion protein and the CD8a or CD80 or both are encoded in a single construct or continuous genomic segment. In some embodiments, the fusion protein and CD8a or CD80 or both are all encoded in a single open reading frame. In some embodiments, the host cell does not replicate for more than 5, 6, 7 8, 9, 10, 12, 14, 16, 18, 24, 36, or 48 hours in the absence of exogenous cytokines. In some embodiments, the host cell is a hematopoietic progenitor cell or human immune cell. In some embodiments, the host cell is a human immune cell, wherein the human immune cell comprises a T cell, a NK cell, a NK-T cell, a dendritic cell, a macrophage, a monocyte, or any combination thereof. In some embodiments, the human immune cell is a T cell, wherein the T cell comprises a CD4⁺ T cell, a CD8⁺ T cell, a CD4⁻ CD8⁻ double negative T cell, a γδ T cell, a naïve T cell, a central memory T cell, a stem cell memory T cell, an effector memory T cell, or any combination thereof.

In some aspects, the present disclosure provides for a method for treating a cancer in a subject, comprising administering to the subject an effective amount of any of the host cells described herein. In some embodiments, the host cell further comprises a TCR directed against an antigen displayed by said cancer. In some embodiments, the cancer is a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Myelomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatosis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma. In some embodiments, the effective amount of the host cell is administered to the subject parenterally or intravenously. In some embodiments, the effective amount comprises about 10⁴ cells/kg to about 10¹¹ cells/kg. In some embodiments, the effective amount comprises CD4⁺ T cells and CD8⁺ T cells. In some embodiments, the method further comprises administering a cytokine to the subject. In some embodiments, the cytokine comprises IL-2, IL-15, or IL-21. In some embodiments, the subject has received or is receiving an immune checkpoint inhibitor and/or an agonist of a stimulatory immune checkpoint agent. In some embodiments, the subject has received myeloablation therapy. In some embodiments, the cancer is reduced by at least 50%, at least 60%, at least 70%, at least 80%, or at least 90% in a period following administering the effective amount of the host cell. In some embodiments, the period comprises fewer than or equal to 120 days, fewer than or equal to 60 days, fewer than or equal to 50 days, fewer than or equal to 40 days, fewer than or equal to 30 days, or fewer than or equal to 20 days. In some embodiments, the method further comprises administering at least a second dose. In some embodiments, the host cells have been validated by any of the methods described in Table 3.

In some aspects, the present disclosure provides for a composition comprising a plurality of host cell, wherein the host cells comprise T-cells directed against, or specific for, a neoantigen (e.g. a mutant KRAS peptide) wherein the composition: (a) comprises at least 10%, 15%, 20%, 25%, 30%, 35%, 40%, 45%, 50%, 55%, 60%, 65%, 70%, 75%, 80%, 85%, 90%, or greater CD3+ cells that stain with dextramer specific for mutant KRAS peptide as assessed by flow cytometry; (b) comprises at least 80%, 85%, 90%, 92%, 94%, 96%, 98%, or greater T cells that are CD3-positive as assessed by flow cytometry; (c) comprises at least 70%, 75%, 80%, 85%, 90%, or greater viable cells as assessed by automated cell counting. In some embodiments, the host cells are any of the host cells described herein. In some embodiments, the composition comprises at least 10%, 15%, 20%, 25%, 30%, 35%, 40%, 45%, 50%, 55%, 60%, 65%, 70%, 75%, 80%, 85%, 90%, or greater CD3+ cells that stain with dextramer specific for mutant KRAS G12V peptide as assessed by flow cytometry. In some embodiments, the composition comprises at least 80%, 85%, 90%, 92%, 94%, 96%, 98%, or greater T cells that are CD3-positive as assessed by flow cytometry. In some embodiments, the composition further comprises a pharmaceutically acceptable excipient.

In some aspects, the present disclosure provides for any of the host cells or any of the vectors described herein and a pharmaceutically acceptable excipient.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A, 1B, 1C, 1D, and 1E relate to identification of KRAS G12V-specific T cell receptors (TCRs) from the T cell repertoire of healthy human donors. (FIG. 1A) (left) Schematic showing a process for identifying HLA-A11-restricted mutant KRAS (mKRAS)-specific T cell lines from donor samples and (right) TNFα production by CD8+ T cells expressing a mKRAS-specific TCR in the absence (left) or presence (right) of mKRAS G12V peptide. (FIG. 1B) Schematic diagrams of processes for (top) sorting and sequencing mKRAS-reactive CD8+ T cells and (bottom) engineering CD8+ T cells to heterologously express a mKRAS-specific TCR. Fifty-six mKRAS-specific TCRs (G12V-specific or G12D-specific) were isolated, and sensitivity and cytotoxicity assays were performed. (FIG. 1C) Fold-enrichment of T cell clones in vitro with and without KRAS G12V mutant peptide. (FIG. 1D) Activation of TCR-transduced T cells in vitro as assessed by the percentage of T cells expressing GFP under the control of Nur77 locus, in the presence of varying concentrations of KRAS G12V mutant peptide. T cells were transduced to express a TCR as shown in the figure key. (FIG. 1E) Log EC50 KRAS G12V 9-mer peptide values (representing the concentration of KRAS G12V peptide required for TCR-transduced T cells to produce their half-maximal response of Nur77 expression).

FIGS. 2A, 2B, and 2C show functional avidity of TCR 11NA4 (see Table 1) compared with that of TCR 220_21 (V-domain amino acid sequences sh7own in SEQ ID NOs:61 (Vα) and 62 (Vβ)) and TCR “BNT” (Vα domain amino acid sequence (with signal peptide) shown in SEQ ID NO:60; Vβ domain amino acid sequence (with signal peptide) shown in SEQ ID NO:59). (FIG. 2A) Percent of TCR-transduced primary CD8+ T cells expressing CD137 at the indicated concentrations of KRAS G12V peptide; (FIG. 2B) log EC50 of the TCRs for KRAS G12V peptide; (FIG. 2C) T cell activation as measured by percent of TCR-transduced primary CD8+ T cells expressing CD137 at the indicated concentrations of KRAS G12V peptide. (FIG. 2D) log EC50 of the TCRs for KRAS G12V exposed to 9-mer and 10-mer peptides; (FIG. 2E) T cell activation as measured by percent of TCR-transduced primary CD8+ T cells expressing CD137 after exposure to the indicated KRAS G12 peptide. (FIG. 2F) Percent of TCR-transduced primary CD8+ T cells expressing IFN-γ at the indicated concentrations of KRAS G12V peptide.

FIGS. 3A and 3B show activation of TCR-transduced T cells (assessed by percentage of TCR-transduced T cells expressing CD137) cocultured with HLA-A11+ KRAS G12V-expressing tumor cell lines. (FIG. 3A) shows activation of T cells expressing one of four different TCRs in multiple cell lines and in the presence of KRAS peptide comprising the G12V mutation. “UT”=Untransduced, negative control. (FIG. 3B) shows superior activation of T cells expressing the TCR 11N4A relative to other TCRs. “UNTR”—Untransduced, negative control.

FIGS. 4A and 4B relate to specific killing of HLA-A11+ KRAS G12V-expressing tumor cell lines by CD8+T cells expressing a KRAS G12V-specific TCR in an Incuyte killing assay. In this assay, the Red Object Area indicates the presence of tumor cells. (FIG. 4A) mKRAS+/HLA-A11+ tumor cell growth curves in an IncuCyte® killing assay. Tested conditions were tumor cells only, tumor cells+T cells transduced to express TCR 11N4A, and tumor cells transduced to express comparator TCR 220_21. The red object area on the y-axis shows tumor cell growth. Additional tumor cells were added at 72 h. (FIG. 4B) Data from another killing assay experiment in which T cells and SW480 tumor cell line were co-cultured at the indicated effector:target ratios.

FIGS. 5A, 5B, and 5C relate to mutagenesis scanning experiments using KRAS G12 9-mer and 10-mer peptides to characterize the peptide binding motif of TCR 11N4A. (FIG. 5A) Percent of TCR-transduced T cells expressing Nur77-GFP when in the presence of G12V peptide or a variant of the G12V peptide with the amino acid at the indicated position replaced with alanine, glycine, or threonine, as indicated. Left: results from mutational scanning of KRAS G12 9-mer peptide. Right: results from mutational scanning of KRAS G12 10-mer peptide. (FIG. 5B) Percentage of TCR 11N4A-transduced CD8+ T cells expressing the activation marker Nur77 (linked to a reporter gene) when in the presence of the indicated 9-mer peptide. (FIG. 5C) Results from searching the human proteome using ScanProsite (prosite.expasy.org/scanprosite/) using the search string: x-V-G-A-x-G-x-x-K (SEQ ID NO:4). Peptides from the human proteome were scored for predicted binding to HLA-A11.

FIGS. 6A, 6B, 6C, 6D, 6E, 6F, 6G, and 6H show that TCR 11N4A has a low risk of autoreactivity in humans. XScan analysis predicted a single peptide RAB7B that may have potential off-target reactivity in the genome. However, RAB7B peptide failed to stimulate transduced CD4/CD8 T cells at physiologic concentrations demonstrating lack of autoreactivity. (FIG. 6A, FIG. 6B) Reactivity of TCR 11NA4-transduced T cells to a panel of potentially cross-reactive peptides (see FIG. 5B). (FIG. 6C) Peptide dose-response curve of cells transduced to express TCR 11N4A and exposed to KRAS G12V or RAB7B peptide and (FIG. 6D) calculated negative log EC50 of TCR 11NA4-transduced T cells against RAB7B peptide versus cognate KRAS G12V peptide. (FIG. 6E) Percentage of TCR 11N4A-transduced CD8+ T cells expressing CD137 in response to overnight culture with a comprehensive panel of positional scanning peptides containing a substitution of every possible amino acid at each position of the cognate KRAS G12V peptide (172 peptides). Peptides that elicited a response of greater than 15% were considered positive in this assay. (FIG. 6F) Potentially cross-reactive peptides identified from searching ScanProsite for the potentially cross-reactive motif identified from the data FIG. 6E. (FIG. 6G) CD137 expression (determined by flow cytometry) by sort-purified primary CD8⁺ T cells transduced to express TCR 11N4A or TCR 11N4A+CD8αβ and cultured overnight with 100 ng/ml potentially cross-reactive peptide. (FIG. 6H) Similar to the results shown in FIG. 6C, CD8+ T cells lentivirally transduced with A11 G12V TCR, CD8α/CD8β, and FAS-41BB fusion protein are not stimulated following titrated RAB7B peptide incubation (bottom line) and is stimulated following titrated KRAS mutant G12V peptide incubation (top line).

FIGS. 7A and 7B relate to assessing potential alloreactivity of TCR 11N4A. (FIG. 7A) B lymphoblastoid cell line (B-LCL) expressing different HLA alleles were incubated with TCR 11N4A-transduced CD8+ T cells and the T cells were assessed for reactivity, as determined by expression of IFN-γ or CD137. (FIG. 7B) Results from the alloreactivity screen: percent of CD137+ TCR 11N4A-transduced T cells with (top) or without (bottom) co-expression of CD8αβ against B-LCLs expressing common HLA alleles.

FIG. 8 shows killing activity of CD8+ and CD4+ T cells engineered to express TCR 11N4A and a CD8αβ co-receptor (e.g. exogenous CD8αβ co-receptor) against mKRAS:HLA-A11+ tumor cells.

FIGS. 9A, 9B, 9C, 9D, 9E, 9F, 9G, and 9H show nucleotide (FIG. 9A-FIG. 9E) and amino acid (FIG. 9F-FIG. 9H) sequences relating to TCR 11N4A and expression constructs encoding or comprising the same.

FIGS. 10A, 10B, 10C, 10D, 10E, and 10F show nucleotide (FIG. 10A-FIG. 10C) and amino acid (FIG. 10D-FIG. 10F) sequences relating to TCR 11N6 and expression constructs encoding or comprising the same.

It will be understood that not all the sequences shown in FIGS. 9A-10F contains every sequence feature indicated in the key. In the figure keys, the CDR3 sequences are shown in accordance with the IMGT junction definition.

FIG. 11 demonstrates that cells transduced with a single lentiviral construct bearing TCR 11N4A, CD8 αβ co-receptors, and FAS/41BB fusion successfully express all three markers. Shown are representative flow cytometric plots of engineered TCR expression (G12V Tetramer, top), FAS-41BB fusion protein (FAS, middle), and exogenous CD8 (CD8 gated via CD4+, bottom) in primary human CD4/CD8 T cells either untransduced (left) or engineered to express A11 G12V TCR+CD8αβ+FAS-41BB (right). Intracellular 2A staining (x-axis) identified transduced cells via 2A elements that separate the individual parameters within the lentiviral construct. CD8 analysis included only CD4+ T cells, thus excluding endogenous CD8+ T cells. T cells activated with anti-CD3/CD28 beads for 2 days, lentivirally transduced, and analyzed by flow cytometry after 3 days of expansion.

FIGS. 12A and 12B demonstrate that cells transduced with TCR 11N4A, CD8α/CD8β co-receptors, and FAS-41BB fusion protein are reactive to endogenous KRAS mutant peptide presented by MHC class I. (FIG. 12A) Shown is a bar graph of CD137 expression on transduced CD4 T cells co-cultured with A11 KRAS G12V mutant cell lines. (FIG. 12B) Shown is a bar graph of CD137 expression on transduced CD8 T cells co-cultured with A11 KRAS G12V mutant cell lines. The cell lines include cell lines SW527, SW620, CFPAC1, COR-L23, DAN-G, and NCI-H441 expressing HLA-A*11:01 and endogenous KRAS mutant G12V. The induced CD137 expression demonstrates reactivity to endogenous KRAS mutant peptide presented by MHC class I.

FIG. 13 demonstrates that a FAS-41BB fusion protein improves KRAS engineered T cell sensitivity of re-stimulated T cells. In this experiment, T-cells comprising the TCR 11N4A against KRAS, CD8α and CD8β co-receptors, and a FAS/41BB fusion protein according to SEQ ID NO: 80 (alongside the indicated controls) were treated with escalating G12V peptide concentration to stimulate the TCR and the percentage of cells stimulated to express the CD137 receptor were assessed. Inclusion of the FAS-41BB fusion protein effectively increased the magnitude of the stimulatory response of the G12V peptide.

FIGS. 14A-14E demonstrate that a FAS-41BB fusion protein improves KRAS engineered T-cell tumor killing in vitro (e.g., in cell lines expressing Fas ligand). FIG. 14A shows the confluence of SW527 after being co-cultured with untransduced T cells, primary CD4 and CD8 T cells transduced with TCRKRASG12V (11N4A)+CD8α/β co-receptor or with TCRKRASG12V, CD8α/β, and FAS-41BB at a 5:1 or a 2:1 Effector:Target ratio. FIG. 14B is a graph summarizing the results of an experiment in which untransduced T cells (UTD), T cells from Donor 1 transduced with TCRKRASG12V+CD8α/β co-receptor or T cells transduced with TCRKRASG12V, CD8α/β, and FAS-41BB were co-cultured with 1×10⁴ HLA-A*11:01 SW620 tumor cells overexpressing FASLG and a NucLight Red fluorescent protein at a 5:1 effector:target ratio for up to 8 days. Cultures were restimulated approximately every 72 hours with equal numbers of tumor cells to mimic chronic antigen stimulation (▴). FIG. 14C shows the results of the same experiment using T cells from a different donor. FIG. 14D shows the results of the same experiment using T cells from Donor 1 and co-culturing these cells with COR-L23 tumor cells. FIG. 14E shows the results of the same experiment in FIG. 14D using T cells from a different donor. Two different donors were tested within the same study. Tumor confluence as measured by total NucLight Red object area is reported as a metric of tumor cell growth/viability throughout the study.

FIG. 15A demonstrates that a FAS-41BB fusion protein improves expansion of KRAS TCR bearing cells in an in vitro re-challenge assay. Shown in the left panel of the figure is a scheme whereby T-cells comprising the TCR 11N4A against KRAS, CD8αβ co-receptor, and a FAS/41BB fusion protein according to SEQ ID NO: 80 (alongside the indicated controls) were co-cultured with SW527 cells for 3-4 days, followed by counting and transfer to a fresh cell plate of SW527 cells; repeating transfer to fresh plates of SW527 cells repeatedly as indicated. In the right panel is shown a graph of the expansion of the transferred T cells over time. As can be seen in the right panel graph, FAS-41BB fusion protein inclusion with KRAS TCRs improves replication of KRAS TCR bearing cells.

FIG. 15B demonstrates that expansion of KRAS TCR-, CD8α/CD8β-, and FAS-41BB fusion protein-bearing cells in an in vitro re-challenge assay is improved when the cells comprise both CD4⁺ and CD8⁺ cells. Shown is a plot of accumulated fold expansion of CD4+ (triangle; the middle line), CD8+ (square; the 2^{nd} from bottom line), CD4+/CD8+ mixture (circle; the top line), or corresponding untransduced control (the bottom line) primary T cells in co-culture with SW527 cell line expressing HLA-A*11:01 and endogenous KRAS mutant G12V.

FIG. 15C shows TCR-engineered cells from two different healthy donors (D1, D2) or untransduced donor T cells (UTD) that were co-cultured with 1×10⁴ various HLA-A*11:01⁺ KRASG12V+ tumor cells at a 5:1 effector:target ratio for 7 days during which time fresh tumor cells were added twice into the coculture to restimulate the T cells. On day 7, T cell proliferation was measured by flow cytometric propidium iodine (PI) staining of CD4+ and CD8+ T cells. PI negative T cell counts are plotted as Live Lymphocyte count/μL.

FIG. 16A demonstrates that a FAS-41BB fusion protein improves efficacy of KRAS TCR bearing cells in an in vivo xenograft tumor model with SW527 cells. In this experiment, T-cells comprising the TCR 11N4A against KRAS, CD8αβ co-receptor (e.g. exogenous CD8αβ co-receptor), and a FAS/41BB fusion protein according to SEQ ID NO: 80 (alongside the indicated controls) were administered at a dose of 10 million T-cells intravenously to immunodeficient mice bearing subcutaneous SW527 tumors and tumor volume was measured over time. As can be seen by the graph, Fas/41BB fusion protein inclusion with KRAS TCRs improves killing of the SW527 tumors in vivo beyond that of cells lacking the Fas/41BB fusion protein.

FIG. 16B demonstrates that tumor-bearing mice administered cells transduced with TCR 11N4A, CD8αβ co-receptor (e.g. exogenous CD8αβ co-receptor), and FAS/41BB fusion protein have superior survival in vivo versus cells transduced with TCR 11N4A and CD8αβ co-receptor (e.g. exogenous CD8αβ co-receptor)s without FAS/41BB fusion.

FIG. 16C demonstrates a complete response has been achieved in certain mice with SW527 tumor cell subcutaneous inoculation received a single intravenous administration of about 1×10⁷ primary CD4/CD8 T cells lentivirally transduced with A11 G12V TCR, CD8αβ co-receptor (e.g. exogenous CD8αβ co-receptor), and FAS-41BB (bottom lines) compared to untransduced T cells (top lines).

FIG. 16D demonstrates that tumor-bearing mice administered cells transduced with TCR 11N4A, CD8αβ co-receptor (e.g. exogenous CD8αβ co-receptor), and FAS/41BB fusion protein show enhanced survival relative to mice administered untransduced cells. Shown is a Kaplan-Meier survival curve of tumor-bearing mice following administration of engineered CD4/CD8 T cells. Shown is the probability of survival of mice bearing SW527 xenografts expressing HLA-A*11:01 and endogenous KRAS mutant G12V. Lines depict tumor-bearing mice receiving primary CD4/CD8 T cells either untransduced (grey) or lentivirally transduced with A11 G12V TCR, CD8αβ co-receptor (e.g. exogenous CD8αβ co-receptor), and FAS-41BB (top flat line). Cells were expanded for 7 days with anti-CD3/CD28 beads following transduction. To initiate the experiment, ten million transduced T cells were intravenously administered 10 days following SW527 cell subcutaneous inoculation when tumor reached approximately 100 mm³. T cells were cryopreserved and thawed prior to administration.

FIG. 17A-17D demonstrate that KRAS TCR-, CD8α/CD8β-, and FAS-41BB fusion protein-bearing cells show improved anti-tumor activity when they comprise both CD4⁺ and CD8⁺ cells. FIG. 17A is a plot of confluence of SW527 tumor cell line expressing a red fluorescent protein, HLA-A*11:01, and endogenous KRAS mutant G12V monitored in a live tumor-visualization assay quantifying red fluorescence signal over time. Cultures comprised a SW527 monoculture (grey) or were co-cultured with untransduced CD4+/CD8+ mixed T cells (black), or CD4+ (red), CD8+ (blue), or CD4+/CD8+ mixed (green) T cells lentivirally transduced with A11 G12V TCR+CD8αβ+FAS-41BB. Primary T cells were activated with anti-CD3/CD28 beads, expanded for 5 days following transduction, co-cultured with SW527 cells at an initial ratio of 0.5:1, and every 3 days (indicated by arrow) additional fresh SW527 cells were added to the culture. FIG. 17B is a plot summarizing the results of the same experiment performed in FIG. 17A but in SW620 cells. FIG. 17C is a plot summarizing the results of the same experiment performed in FIG. 17A but in CFPAC1 cells. FIG. 17D is a plot summarizing the results of the same experiment performed in FIG. 17A but in COR-L23 cells.

FIG. 18 demonstrates that cells transduced with TCR 11N4A, CD8αβ co-receptor (e.g. exogenous CD8αβ co-receptor), and FAS/41BB fusion protein fail to proliferate in the absence of exogenous cytokine support, enhancing their safety profile. Shown is a plot of persistence (measured by cell count) of CD4+/CD8+ T cells monitored by quantifying cells every 2-4 days in absence of exogenous cytokines. Shown are primary T cells either untransduced (grey line; top line) or transduced with A11 G12V TCR+CD8αβ+FAS-41BB (bottom line) that have been expanded with anti-CD3/CD28 beads in media containing IL2/IL7/IL15 for 7-10 days and transferred to media without cytokine. Half of the media (without cytokine) was replenished every 2-4 days.

FIG. 19 illustrates several designs for lentiviral vectors that comprise anti-KRAS TCR, FAS-41BB fusion protein, and CD8a/CD80. Most of the designs contemplated expressing anti-KRAS TCR (“TCRb” or “TCRa”), CD8α/CD8β (“CD8a” or “CD8b”), and FAS-41BB (“FasBB”) on a single translated RNA under a single promoter (“MSCV”, or Murine Stem Cell Virus promoter) with the usage of in-frame sequences encoding self-cleaving peptides (“P2A”, “T2A”, “F-P2A”) separating regions encoding the separate polypeptides. Some (22992-8, 22992-9) involve expression of Fas-41BB under a separate promoter (“PGK” or phosphoglycerate kinase promoter).

FIG. 20 demonstrates that T cells generated by a manufacturing strategy that involves a single vector comprising anti-KRAS TCR, FAS-41BB fusion protein, and CD8α/CD8β show superior TCR expression and surface activity versus cells generated by a strategy that involves anti-KRAS TCR and FAS-41BB fusion proteins on separate vectors. FIG. 20A shows alternate designs of the lentiviral vector. FIG. 20B shows FACS analyses of T cells transduced as described previously with the generated lentiviral vectors. FIG. 20C shows the percentage of cells expressing a cistron comprising the anti-KRAS TCR (“2A+%”), the percentage of cells expressing functional TCR and a cistron comprising the anti-KRAS TCR (“Tet+2A+%”), overall functional TCR expression (“Tet MFI”), FAS-41BB fusion protein expression (“Fas MFI”), and CD8α/CD8β coreceptor expression by CD4+ cells (“CD8 MFI under CD4+”). The FACS analysis indicated that in terms of TCR and CD8 expression, the single lentiviral strategy (“22992-4”) was superior to the dual lentiviral strategy (“2 lentivirus”)

FIG. 21A shows the activation of T cells generated by a manufacturing strategy that involves a single vector comprising anti-KRAS TCR, FAS-41BB fusion protein, and CD8α/CD8β or a dual vector system.

FIG. 21B shows the cell killing activity of these cells when administered as fresh TCR-T cells or after thawing in various tumor cell lines.

FIG. 22A shows long term repeat stimulation and tumor cell killing of T cells generated by a manufacturing strategy that involves a single vector comprising anti-KRAS G12V TCR, FAS-41BB fusion protein, and CD8α/CD8β or a dual vector system.

FIG. 22B shows the changes in tumor cell volume after administration of these cells in in vivo xenograft models.

FIG. 22C shows the changes in tumor cell volume after administration of cells comprising an anti-KRAS G12D TCR, FAS-41BB fusion protein, and CD8α/CD8β in in vivo xenograft models.

### DETAILED DESCRIPTION

Effective T cell activation often requires or is enhanced by a concurrent co-stimulatory signal. In the tumor microenvironment, co-stimulatory molecules are generally downregulated. Accordingly, there is a need for configurations of cells used for adoptive T cell therapy that counteract this downregulation of co-stimulatory molecules or generally enhance the effect of antigen-targeted receptors on such T cells in the tumor microenvironment. Additionally, the tumor microenvironment may comprise heterogenous cell types (e.g., stromal cells, endothelial cells, and tumor-associated macrophages, granulocytes, and inflammatory monocytes) which contribute to T cell suppression through direct contact and secretion of soluble inhibitory factors.

Some aspects of the present disclosure generally relate to cells (e.g., immune effector cells such as CD4+ and/or CD8⁺ T cells) that express 1) an exogenous binding protein that binds to a neoantigen peptide:HLA complex, 2) a fusion protein (e.g., Fas-41BB fusion protein), and 3) a CD8αβ co-receptor (e.g. exogenous CD8αβ co-receptor). Some aspects of the present disclosure generally relate to one or more constructs encoding 1) an exogenous binding protein that binds to a neoantigen peptide:HLA complex, 2) a fusion protein (e.g., Fas-41BB fusion protein), and 3) a CD8αβ co-receptor (e.g. exogenous CD8αβ co-receptor).

Some aspects of the present disclosure generally relate to fusion proteins (e.g., fusion receptors or “switch” receptors) that convert T cell inhibitory signals in the tumor microenvironment into T cell activating or proliferatory signals. Accordingly, some aspects of the disclosure relate to fusion proteins comprising an extracellular domain specific for soluble or cell-anchored inhibitory ligands linked to an intracellular domain that contributes to T-cell activation (e.g., a 4-1BB intracellular signaling domain, or a CD28 intracellular signaling domain). In some cases, such proteins comprise an extracellular domain derived from a Fas receptor and an intracellular domain derived from a 4-1BB receptor (e.g., Fas-41BB fusion proteins).

Without wishing to be bound by theory, such Fas-41BB fusion proteins may inhibit T cell apoptosis, enhance IL-2 or IFN-γ secretion, favor memory T cell development, increase T cell metabolic capacity, and/or improve T cell proliferation, persistence and fitness through NF-κB activation, increased Bcl-2 expression, and PI3K and MEK-1/2 signaling pathway activation in response to Fas ligand (FASLG) in the tumor microenvironment. Alternatively or additionally, such Fas-41BB fusion proteins may act in a dominant negative fashion or sequester Fas ligand expression by tumors, endothelium, and stimulated T cells in the tumor microenvironment, preventing elimination or apoptosis of T cells upon tumor infiltration. Fas ligand has been documented to be expressed in the tumor microenvironment of many solid tumors, and it is contemplated that the presence of Fas ligand in the microenvironment of solid tumors may contribute to limited efficacy of T cell adoptive cell therapy.

Some aspects of the present disclosure generally relate to binding proteins specific for Ras neoantigens, modified immune cells expressing the same, polynucleotides that encode the binding proteins, and related uses. Mutated Ras proteins (e.g., KRAS, NRAS, HRAS) can produce neoantigens, including a G→V mutation at position 12 of the full-length KRAS protein (SEQ ID NO: 1; UniProt KB P01116) or at position 12 of the full-length NRAS protein (SEQ ID NO: 78; Uniprot KB P01111) or at position 12 of the full-length HRAS protein (SEQ ID NO:79; Uniprot KB P01112).

Some aspects of the present disclosure generally relate to binding proteins specific for p53 neoantigens, modified immune cells expressing the same, polynucleotides that encode the binding proteins, and related uses. Mutated p53 proteins can potentially produce neoantigens; for example, at positions R175, G245, R248, R249, R273 and R282 (relative to SEQ ID NO:1039 (wild type p53). Missense mutations account for approximately 70%-80% of p53 mutations, and downregulation of wild type p53 activity occurs in most, if not all, human malignancies (Duffy et al., Seminars Cancer Bio., 79:58-67 (2022).

Some aspects of the present disclosure generally relate to binding proteins specific for PIK3CA neoantigens, modified immune cells expressing the same, polynucleotides that encode the binding proteins, and related uses. Mutated p53 proteins can potentially produce neoantigens; for example, at positions R38, G106, C420, E453, E542, E545, M1043, and H1047 (relative to SEQ ID NO:1040 (wild type PIK3CA). Missense mutations account for approximately 70%-80% of PIK3CA mutations, and mutations in PIK3CA activity have been found in many human cancers (Ligresti et al., Cell Cycle, 8(9):1352-58 (2009).

In the present disclosure, binding proteins that are capable of binding to neoantigens are provided. In certain aspects, binding proteins (and host cells, such as immune cells, that comprise a heterologous polynucleotide that encodes a binding protein of the present disclosure) are provided that comprise a TCR Vα domain and a TCR VP domain, wherein the binding proteins are capable of binding to a neoantigen peptide:HLA complex.

For example, in the present disclosure, binding proteins that are capable of binding to Ras neoantigens are provided. In certain aspects, binding proteins (and host cells, such as immune cells, that comprise a heterologous polynucleotide that encodes a Ras-specific binding protein of the present disclosure) are provided that comprise a TCR Vα domain and a TCR Vβ domain, wherein the binding proteins are capable of binding to a Ras peptide antigen:HLA complex, wherein the Ras peptide antigen comprises, consists essentially of, or consists of the amino acid sequence set forth in any one of SEQ ID NOs:2 or 3. In certain embodiments, the HLA comprises HLA-A*11, such as HLA-A*11:01.

Disclosed binding proteins are highly sensitive to antigen, capable of inducing activation of host T cells at low concentrations of peptide antigen. In certain embodiments, of a population or sample of (e.g., CD8+ and/or CD4+) T cells expressing a binding protein have half-maximal expression of the activation marker Nur77 when in the presence of [Log EC50 less than −9 M (e.g., between −9 M and −10 M)] peptide. In certain embodiments, of a population or sample of (e.g., CD8+ and/or CD4+) T cells expressing a binding protein, the T cells have half-maximal expression of CD137 when in the presence of [Log EC50 less than −10 M (e.g., between −10 M and −11 M)]. In certain embodiments, of a population or sample of (e.g., CD8+ and/or CD4+) T cells expressing a binding protein, the T cells have half-maximal expression of IFN-γ when in the presence of [Log EC50 less than −10 M (e.g., between −10 M and −11 M)] peptide.

Host (e.g., T) cells expressing a binding protein according to the present disclosure are activated (e.g., as determined by expression of CD137) in the presence of a neoantigen to which the binding protein recognizes. For example, a binding protein that recognizes and binds a mutant KRAS is activated in the presence of mutant KRAS-expressing cancer cell lines (e.g., OVCAR5 (ovarian serous adenocarcinoma), DAN-G (pancreatic adenocarcinoma), CFPAC1 (pancreatic adenocarcinoma), SW480 (colon carcinoma), SW527 (breast carcinoma), and NCI-H441 (lung adenocarcinoma) cell lines).

In some embodiments, host cells (e.g., T cells, such as CD4+ T cells or CD8+ T cells) expressing a binding protein according to the present disclosure are capable of specifically killing cells expressing a neoantigen (e.g., mutant KRAS-expressing cells (e.g., SW480 cells, such as at an 8:1 effector:target ratio, a 4:1 effector:target ratio, or a 2:1 effector:target ratio)). In some embodiments, the host cells expressing a binding protein according to the present disclosure are capable of specifically killing cells expressing a neoantigen (e.g., mutant KRAS-expressing cells) for over 144 hours in vitro, including when additional tumor cells are added at 72 hours in a re-challenge setting.

In certain embodiments, binding proteins of the present disclosure are non-alloreactive against, are substantially non-alloreactive against, and/or have a low risk of alloreactivity against (i) amino acid sequences from the human proteome and/or (ii) against human HLA alleles.

In any of the herein disclosed embodiments, a binding protein can be human, humanized, or chimeric. Also provided are polynucleotides that encode a binding protein, vectors that comprise a polynucleotide, and host cells that comprise a polynucleotide and/or vector and/or that express a binding protein. Presently disclosed binding proteins, and host cells (e.g., T cells, NK cells, NK-T cells) are useful for treating a disease or disorder associated with a KRAS neoantigen, such as, for example, a cancer. Presently disclosed binding proteins can also bind to G12V antigens arising in human NRAS or human HRAS, which proteins comprise an identical sequence to KRAS in the region near residue G12. Accordingly, the disclosed compositions are useful in treating disease or disorders associated with a KRAS neoantigen, with a NRAS neoantigen comprising a G12V mutation, or with a HRAS neoantigen comprising a G12V mutation, or any combination thereof.

Also provided are methods and uses of the presently disclosed binding proteins, polynucleotides, vectors, host cells, and related compositions, for the treatment of a disease or disorder associated with a neoantigen (e.g., KRAS, NRAS, HRAS, p53, and/or PIK3CA) mutation as provided herein.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include”, “have”, and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity or avidity of a binding protein).

As used herein, “protein” or “polypeptide” generally refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers. In some embodiments, a “peptide” (e.g., a peptide antigen) refers to a polymer of about 8-10 amino acid residues in length.

As used herein, a “hematopoietic progenitor cell” generally refers to a cell that can be derived from hematopoietic stem cells or fetal tissue and is capable of further differentiation into mature cell types (e.g., immune system cells). Exemplary hematopoietic progenitor cells include those with a CD24^{Lo} Lin⁻ CD117⁺ phenotype or those found in the thymus (referred to as progenitor thymocytes).

As used herein, an “immune system cell” generally refers to any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4⁺ T cell, a CD8⁺ T cell, a CD4⁻ CD8⁻ double negative T cell, a γδ T cell, a regulatory T cell, a natural killer cell, a natural killer T cell, and a dendritic cell. Macrophages and dendritic cells can be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” generally refers to an immune system cell that matures in the thymus and produces a T cell receptor (TCR). T cells can be naïve (“TN”; not exposed to antigen; increased expression of CD62L, CCR7, CD28, CD3, CD127, and CD45RA, and decreased or no expression of CD45RO as compared to T_{CM} (described herein)), memory T cells (T_{M}) (antigen experienced and long-lived), including stem cell memory T cells, and effector cells (antigen-experienced, cytotoxic). T_{M} can be further divided into subsets of central memory T cells (T_{CM} expresses CD62L, CCR7, CD28, CD95, CD45RO, and CD127) and effector memory T cells (T_{EM} express CD45RO, decreased expression of CD62L, CCR7, CD28, and CD45RA). Effector T cells (T_{E}) refers to antigen-experienced CD8⁺ cytotoxic T lymphocytes that express CD45RA, have decreased expression of CD62L, CCR7, and CD28 as compared to T_{CM}, and are positive for granzyme and perforin. Helper T cells (T_{H}) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other example T cells include regulatory T cells, such as CD4⁺ CD25⁺ (Foxp3⁺) regulatory T cells and Treg17 cells, as well as Tr1, Th3, CD8⁺ CD28⁻, and Qa-1 restricted T cells.

“T cell receptor” (TCR) generally refers to an immunoglobulin superfamily member having a variable binding domain, a constant domain, a transmembrane region, and a short cytoplasmic tail; see, e. g., Janeway et al., Immunobiology: The Immune System in Health and Disease, 3rd Ed., Current Biology Publications, p. 433, 1997) capable of specifically binding to an antigen peptide bound to a MHC receptor. A TCR can be found on the surface of a cell or in soluble form and generally is comprised of a heterodimer having α and β chains (also known as TCR α and TCRβ, respectively), or γ and δ chains (also known as TCRγ and TCRδ, respectively). In certain embodiments, a polynucleotide encoding a binding protein of this disclosure, e.g., a TCR, can be codon optimized to enhance expression in a particular host cell, such, for example, as a cell of the immune system, a hematopoietic stem cell, a T cell, a primary T cell, a T cell line, a NK cell, or a natural killer T cell (Scholten et al., Clin. Immunol. 119:135, 2006). Exemplary T cells that can express binding proteins and TCRs of this disclosure include CD4⁺ T cells, CD8⁺ T cells, and related subpopulations thereof (e.g., naïve, central memory, stem cell memory, effector memory).

Like other immunoglobulins (e.g., antibodies), the extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.)) at the N-terminus, and one constant domain (e.g., α-chain constant domain or C_{α}, typically 5 amino acids 117 to 259 based on Kabat, β-chain constant domain or C_{β}, typically amino acids 117 to 295 based on Kabat) adjacent the cell membrane. Also, like immunoglobulins, the variable domains contain complementary determining regions (CDRs) separated by framework regions (FRs) (see, e.g., Jores et al., Proc. Nat'l Acad. Sci. USA 87:9138, 1990; Chothia et al., EMBO J. 7:3745, 1988; see also Lefranc et al., Dev. Comp. Immunol. 27:55, 2003). The source of a TCR as used in the present disclosure may be from various animal species, such as a human, mouse, rat, rabbit, or other mammal.

The term “variable region” or “variable domain” generally refers to the domain of an immunoglobulin superfamily binding protein (e.g., a TCR α-chain or β-chain (or γ chain and δ chain for γδ TCRs)) that is involved in binding of the immunoglobulin superfamily binding protein (e.g., TCR) to antigen. The variable domains of the α-chain and β-chain (Vα and Vβ, respectively) of a native TCR generally have similar structures, with each domain comprising four generally conserved framework regions (FRs) and three CDRs. The Vα domain is encoded by two separate DNA segments, the variable gene segment and the joining gene segment (V-J); the Vβ domain is encoded by three separate DNA segments, the variable gene segment, the diversity gene segment, and the joining gene segment (V-D-J). A single Vα or Vβ domain may be sufficient to confer antigen-binding specificity. Furthermore, TCRs that bind a particular antigen may be isolated using a Vα or Vβ domain from a TCR that binds the antigen to screen a library of complementary Vα or Vβ domains, respectively.

The terms “complementarity determining region,” and “CDR,” are generally synonymous with “hypervariable region” or “HVR,” and generally refer to sequences of amino acids within immunoglobulin (e.g., TCR) variable regions. CDRs confer antigen specificity and binding affinity and are separated from one another in primary amino acid sequence by framework regions. In general, there are three CDRs in each TCR α-chain variable region (αCDR1, αCDR2, αCDR3) and three CDRs in each TCR β-chain variable region (βCDR1, βCDR2, βCDR3). In TCRs, CDR3 is thought to be the main CDR responsible for recognizing processed antigen. In general, CDR1 and CDR2 interact mainly or exclusively with the MHC.

CDR1 and CDR2 are encoded within the variable gene segment of a TCR variable region-coding sequence, whereas CDR3 is encoded by the region spanning the variable and joining segments for Vα, or the region spanning variable, diversity, and joining segments for Vβ. Thus, if the identity of the variable gene segment of a Vα or Vβ is known, the sequences of their corresponding CDR1 and CDR2 can be deduced; e.g., according to a numbering scheme as described herein. Compared with CDR1 and CDR2, CDR3, and in particular CDR3α, is typically significantly more diverse due to the addition and loss of nucleotides during the recombination process.

TCR variable domain sequences can be aligned to a numbering scheme (e.g., Kabat, Chothia, EU, IMGT, Enhanced Chothia, and Aho), allowing equivalent residue positions to be annotated and for different molecules to be compared using, for example, ANARC1 software tool (2016, Bioinformatics 15:298-300). A numbering scheme provides a standardized delineation of framework regions and CDRs in the TCR variable domains. In certain embodiments, a CDR of the present disclosure is identified according to the IMGT numbering scheme (Lefranc et al., Dev. Comp. Immunol. 27:55, 2003; imgt.org/IMGTindex/V-QUEST.php). In some embodiments, a CDR (e.g., CDR3) is identified or defined in accordance with the IMGT junction definition. In some embodiments, a CDR (e.g., CDR3) is identified or defined in accordance with the IMGT definition. In some embodiments, a CDR of the present disclosure is identified or defined according to the Kabat numbering scheme or method. In some embodiments, a CDR of the present disclosure is identified or defined according to the Chothia numbering scheme or method. In some embodiments, a CDR of the present disclosure is identified or defined according to the EU numbering scheme or method. In some embodiments, a CDR of the present disclosure is identified or defined according to the enhanced Chothia numbering scheme or method. In some embodiments, a CDR or defined of the present disclosure is identified according to the Aho numbering scheme or method.

The source of a TCR as used in the present disclosure may be from any of a variety of animal species, such as a human, mouse, rat, rabbit, or other mammal. TCR constant domain sequences may be from, for example, human, mouse, marsupial (e.g., opossum, bandicoot, wallaby), shark, or non-human primate. In certain embodiments, TCR constant domain sequences are human or comprise engineered variants of human sequences. TCR constant domains may be engineered to improve pairing, expression, stability, or any combination of these. See, e.g., Cohen et al., Cancer Res, 2007; Kuball et al., Blood 2007; and Haga-Freidman et al., Journal of Immunology 2009. Examples of engineering in TCR Cα and Cβ include mutation of a native amino acid to a cysteine so that a disulfide bond forms between the introduced cysteine of one TCR constant domain and a native cysteine of the other TCR constant domain. Such mutations can include T48C in Cα, T57C in Cβ, or both. Mutations to improve stability can include a mutation in the Cα transmembrane domain from the sequence LSVIGF to the sequence LLVIVL (“L-V-L” mutation; see Haga-Friedman et al., J Immunol 188:5538-5546 (2012), the TCR mutations and mutant TCR constant domain sequences of which are incorporated herein by reference).

As used herein, the term “CD8 co-receptor” or “CD8” generally refers to the cell surface glycoprotein CD8, either as an alpha-alpha homodimer or an alpha-beta heterodimer. The CD8 co-receptor assists in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). There are five (5) human CD8 beta chain isoforms (see UniProtKB identifier P10966) and a single human CD8 alpha chain isoform (see UniProtKB identifier P01732).

“CD4” generally refers to an immunoglobulin co-receptor glycoprotein that assists the TCR in communicating with antigen-presenting cells (see, Campbell & Reece, Biology 909 (Benjamin Cummings, Sixth Ed., 2002)). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII β2, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce or recruit various types of immune system cells, including T helper cells, and immune responses.

In certain embodiments, a TCR is found on the surface of T cells (or T lymphocytes) and associates with a CD3 complex. “CD3” is a multi-protein complex of six chains (see, Abbas and Lichtman, 2003; Janeway et al., p. 172 and 178, 1999) that is associated with antigen signaling in T cells. In mammals, the complex comprises a CD3γ chain, a CD3δ chain, two CD3ε chains, and a homodimer of CD3ζ chains. The CD3γ, CD3β, and CD3ε chains are related cell surface proteins of the immunoglobulin superfamily containing a single immunoglobulin domain. The transmembrane regions of the CD3γ, CD3β, and CD3ε chains are negatively charged, which is believed to allow these chains to associate with positively charged regions of T cell receptor chains. The intracellular tails of the CD3γ, CD3β, and CD3ε chains each contain a single conserved motif known as an immunoreceptor tyrosine-based activation motif or ITAM, whereas each CD3ζ chain has three. Without wishing to be bound by theory, it is believed that the ITAMs are important for the signaling capacity of a TCR complex. CD3 as used in the present disclosure may be from various animal species, including human, mouse, rat, or other mammals.

As used herein, “TCR complex” generally refers to a complex formed by the association of CD3 with TCR. For example, a TCR complex can be composed of a CD3γ chain, a CD3β chain, two CD3ε chains, a homodimer of CD3ζ chains, a TCRα chain, and a TCRβ chain. Alternatively, a TCR complex can be composed of a CD3γ chain, a CD3β chain, two CD3ε chains, a homodimer of CD3ζ chains, a TCRγ chain, and a TCRβ chain.

A “component of a TCR complex”, as used herein, generally refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Chimeric antigen receptor” (CAR) generally refers to a fusion protein that is engineered to contain two or more naturally occurring amino acid sequences, domains, or motifs, linked together in a way that does not occur naturally or does not occur naturally in a host cell, which fusion protein can function as a receptor when present on a surface of a cell. CARs can include an extracellular portion comprising an antigen-binding domain (e.g., obtained or derived from an immunoglobulin or immunoglobulin-like molecule, such as a TCR binding domain derived or obtained from a TCR specific for a cancer antigen, a scFv derived or obtained from an antibody, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016), Stone et al., Cancer Immunol. Immunother., 63(11):1163 (2014), and Walseng et al., Scientific Reports 7:10713 (2017), which CAR constructs and methods of making the same are incorporated by reference herein). CARs of the present disclosure that specifically bind to a Ras antigen (e.g., in the context of a peptide:HLA complex) comprise a TCR Vα domain and a VP domain.

Any polypeptide of this disclosure can, as encoded by a polynucleotide sequence, comprise a “signal peptide” (also known as a leader sequence, leader peptide, or transit peptide). Signal peptides can target newly synthesized polypeptides to their appropriate location inside or outside the cell. In some contexts, signal peptides are from about 15 to about 22 amino acids in length. A signal peptide may be removed from the polypeptide during, or once localization (e.g., membrane insertion) or secretion is completed. Polypeptides that have a signal peptide are referred to herein as a “pre-protein” and polypeptides having their signal peptide removed are referred to herein as “mature” proteins or polypeptides. In any of the herein disclosed embodiments, a binding protein or fusion protein comprises, or is, a mature protein, or is or comprises a pre-protein.

A “linker” generally refers to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In certain embodiments, a linker is comprised of about two to about 35 amino acids, for instance, or about four to about 20 amino acids or about eight to about 15 amino acids or about 15 to about 25 amino acids. Example linkers include glycine-serine linkers.

“Antigen” or “Ag” as used herein generally refers to an immunogenic molecule that provokes an immune response. This immune response may involve antibody production, activation of specific immunologically competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Example biological samples that can contain one or more antigens include tissue samples, tumor samples, cells, biological fluids, or combinations thereof. Antigens can be produced by cells that have been modified or genetically engineered to express an antigen, or that endogenously (e.g., without modification or genetic engineering by human intervention) express a mutation or polymorphism that is immunogenic.

A “neoantigen,” as used herein, generally refers to a host cellular product containing a structural change, alteration, or mutation that creates a new antigen or antigenic epitope that has not previously been observed in the subject's genome (i.e., in a sample of healthy tissue from the subject) or been “seen” or recognized by the host's immune system, which: (a) is processed by the cell's antigen-processing and transport mechanisms and presented on the cell surface in association with an MHC (e.g., HLA) molecule; and (b) elicits an immune response (e.g., a cellular (T cell) response). Neoantigens may originate, for example, from coding polynucleotides having alterations (substitution, addition, deletion) that result in an altered or mutated product, or from the insertion of an exogenous nucleic acid molecule or protein into a cell, or from exposure to environmental factors (e.g., chemical, radiological) resulting in a genetic change. Neoantigens may arise separately from a tumor antigen or may arise from or be associated with a tumor antigen. “Tumor neoantigen” (or “tumor-specific neoantigen”) refers to a protein comprising a neoantigenic determinant associated with, arising from, or arising within a tumor cell or plurality of cells within a tumor. Tumor neoantigenic determinants are found on, for example, antigenic tumor proteins or peptides that contain one or more somatic mutations or chromosomal rearrangements encoded by the DNA of tumor cells (e.g., pancreas cancer, lung cancer, colorectal cancers), as well as proteins or peptides from viral open reading frames associated with virus-associated tumors (e.g., cervical cancers, some head and neck cancers). The terms “antigen” and “neoantigen” are used interchangeably herein when referring to a Ras antigen comprising a mutation as disclosed herein. In some embodiments, a neoantigen comprises a RAS peptide (e.g., KRAS, HRAS, or NRAS), a BRAF peptide, a CALR peptide, a DNMT3A peptide, a EGFR peptide, a ERBB2 peptide, a ESR1 peptide, a FGFR3 peptide, a FLT3 peptide, a GNA11 peptide, a GNAQ peptide, an IDH peptide, an MYD88 peptide, a p53 peptide, a PIK3CA peptide, or an SF3B1 peptide. In some embodiments, a neoantigen comprises an ALK peptide, an EGFR peptide, a HER2 peptide, a KIT peptide, a MET peptide, an NRG1 peptide, an NTRK peptide, a PDGFRα peptide, a RAF peptide, a RET peptide, or a ROS1 peptide.[WH1] This list is not exhaustive as other neoantigens are contemplated. In some embodiments, a neoantigen comprises an oncogenic driver mutation. Without being bound by theory, oncogenic driver mutations are believed to be responsible for the initiation and maintenance of a cancer.

The term “epitope” or “antigenic epitope” generally includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three-dimensional structural characteristics, as well as specific charge characteristics.

As used herein, the term “KRAS (or NRAS or HRAS) antigen (or neoantigen)” or “KRAS (or NRAS or HRAS) peptide antigen (or neoantigen)” or “KRAS (NRAS or HRAS) peptide” generally refers to a naturally or synthetically produced peptide portion of a KRAS or NRAS or HRAS protein ranging in length from about 7 amino acids, about 8 amino acids, about 9 amino acids, about 10 amino acids, up to about 20 amino acids, and comprising at least one amino acid alteration caused by a G12 (e.g., G12V) mutation (wherein position 12 is in reference to the full-length KRAS protein sequence set forth in SEQ ID NO:1; and is also in reference to the full-length NRAS and HRAS protein sequence set forth in SEQ ID NOs: 78 and 79, respectively), which peptide can form a complex with a MHC (e.g., HLA) molecule, and a binding protein of this disclosure specific for a KRAS or NRAS or HRAS peptide:MHC (e.g., HLA) complex can specifically bind to such as complex. An example KRAS (or NRAS or HRAS) antigen comprises, consists essentially of, or consists of a peptide having the amino acid sequence of SEQ ID NO:2 or 3.

“Major histocompatibility complex” (MHC) generally refers to glycoproteins that deliver peptide antigens to a cell surface of all nucleated cells. MHC class I molecules are heterodimers having a membrane spanning α chain (with three a domains) and a non-covalently associated β₂ microglobulin. MHC class II molecules are composed of two transmembrane glycoproteins, α and β, both of which span the membrane. Each chain comprises two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. Human MHC is referred to as human leukocyte antigen (HLA). HLAs corresponding to “class I” MHC present peptides from inside the cell and include, for example, HLA-A, HLA-B, and HLA-C. Alleles include, for example, HLA A*11, such as HLA-A*11:01. HLAs corresponding to “class II” MHC present peptides from outside the cell and include, for example, HLA-DP, HLA-DM, HLA-DOA, HLA-DOB, HLA-DQ, and HLA-DR.

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MHC gene that is relevant for antigen presentation) APC and T cells, are well-established (see, e.g., Murphy, Janeway's Immunobiology (8^{th} Ed.) 2011 Garland Science, NY; chapters 6, 9 and 16). For example, processed antigen peptides originating in the cytosol (e.g., tumor antigen, intracellular pathogen) are generally from about 7 amino acids to about 11 amino acids in length and will associate with class I MHC (HLA) molecules, whereas peptides processed in the vesicular system (e.g., bacterial, viral) will vary in length from about 10 amino acids to about 25 amino acids and associate with class II MHC (HLA) molecules.

The term “KRAS-specific binding protein,” as used herein, generally refers to a protein or polypeptide, such as, for example, a TCR, a scTv, a scTCR, or CAR, that binds to a KRAS peptide antigen or a NRAS peptide antigen or a HRAS peptide antigen (or to a KRAS or NRAS or HRAS peptide antigen:HLA complex, e.g., on a cell surface), and does not bind a peptide that does not contain the KRAS or NRAS or HRAS peptide antigen and does not bind to an HLA complex containing such a peptide.

Binding proteins of this disclosure, such as TCRs, scTCRs, and CARs, contain a binding domain specific for a target. A “binding domain” (also referred to as a “binding region” or “binding moiety”), as used herein, refers to a molecule or portion thereof (e.g., peptide, oligopeptide, polypeptide, protein) that possesses the ability to specifically and non-covalently associate, unite, or combine with a target (e.g., KRAS or NRAS or HRAS peptide or KRAS or NRAS or HRAS peptide:MHC complex). A binding domain includes any naturally occurring, synthetic, semi-synthetic, or recombinantly produced binding partner for a biological molecule, a molecular complex (i.e., complex comprising two or more biological molecules), or other target of interest. Example binding domains include immunoglobulin variable regions or single chain constructs comprising the same (e.g., single chain TCR (scTCR) or scTv).

In certain embodiments, a Ras-specific binding protein binds to a KRAS (or NRAS or HRAS) peptide (or a KRAS (or NRAS or HRAS):HLA complex) with a K_{d} of less than about 10⁻⁸ M, less than about 10⁻⁹ M, less than about 10⁻⁰ M, less than about 10⁻¹¹ M, less than about 10⁻¹² M, or less than about 10⁻¹³ M, or with an affinity that is about the same as, at least about the same as, or is greater than at or about the affinity exhibited by an example Ras-specific binding protein provided herein, such as any of the Ras-specific TCRs provided herein, for example, as measured by the same assay. In certain embodiments, a Ras-specific binding protein comprises a Ras-specific immunoglobulin superfamily binding protein or binding portion thereof.

As used herein “specifically binds” or “specific for” generally refers to an association or union of a binding protein (e.g., TCR receptor) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³ M).

In certain embodiments, a receptor or binding domain may have “enhanced affinity,” which generally refers to a selected or engineered receptors or binding domain with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof.

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

In certain embodiments, a neoantigen (e.g., KRAS (or NRAS, or HRAS), p53, and/or PIK3CA)-specific binding domain alone (i.e., without any other portion of a neoantigen (e.g., KRAS (or NRAS, or HRAS), p53, and/or PIK3CA)-specific binding protein) can be soluble and can bind to neoantigen (e.g., KRAS (or NRAS, or HRAS), p53, and/or PIK3CA) (or a neoantigen (e.g., KRAS (or NRAS, or HRAS), p53, and/or PIK3CA) peptide, or a neoantigen (e.g., KRAS (or NRAS, or HRAS), p53, and/or PIK3CA) peptide:HLA complex) with a K_{d} of less than about 10⁻⁸ M, less than about 10⁻⁹ M, less than about 10⁻¹⁰ M, less than about 10⁻¹¹ M, less than about 10⁻¹² M, or less than about 10⁻¹³ M. In particular embodiments, a neoantigen (e.g., KRAS (or NRAS, or HRAS), p53, and/or PIK3CA)-specific binding domain includes a neoantigen (e.g., KRAS (or NRAS, or HRAS), p53, and/or PIK3CA)-specific scTCR (e.g., single chain αβTCR proteins such as Vα-L-Vβ, Vβ-L-Vα, Vα-Cα-L-Vα, or Vα-L-Vβ-Cβ, wherein Vα and Vβ are TCRα and β variable domains respectively, Cα and Cβ are TCRα and β constant domains, respectively, and L is a linker, such as a linker described herein).

The term “functional avidity”, as used herein, generally refers to a biological measure or activation threshold of an in vitro immune cell (e.g., T cell, NK cell, NK-T cell) response to a given concentration of a ligand, wherein the biological measures can include cytokine production (e.g., IFN-γ production, IL-2 production, etc.), cytotoxic activity, activation markers (e.g., CD137, Nur77) and proliferation. For example, T cells that biologically (immunologically) respond in vitro to a low antigen dose by, for example, producing cytokines, exhibiting cytotoxic activity, or proliferating are considered to have high functional avidity, while T cells having lower functional avidity require higher amounts of antigen before an immune response, similar to the high-avidity T cells, is elicited. It will be understood that functional avidity is different from affinity and avidity. Affinity refers to the strength of any given bond between a binding protein and its antigen/ligand. Some binding proteins are multivalent and bind to multiple antigens—in this case, the strength of the overall connection is the avidity.

Numerous correlations exist between the functional avidity and the effectiveness of an immune response. Some ex vivo studies have shown that distinct T cell functions (e.g., proliferation, cytokines production, etc.) can be triggered at different thresholds (see, e.g., Betts et al., J. Immunol. 172:6407, 2004; Langenkamp et al., Eur. J. Immunol. 32:2046, 2002). Factors that affect functional avidity can include (a) the affinity of a TCR for the pMHC-complex, that is, the strength of the interaction between the TCR and pMHC (Cawthon et al., J. Immunol. 167:2577, 2001), (b) expression levels of the TCR, and, in some embodiments, CD4 or CD8 co receptors, on the host cell and (c) the distribution and composition of signaling molecules (Viola and Lanzavecchia, Science 273:104, 1996), as well as expression levels of molecules that attenuate T cell function and TCR signaling.

The concentration of antigen needed to induce a half-maximum response (e.g., production of a cytokine or activation marker by a host cell; fluorescence intensity when binding to a labeled peptide:HLA multimer) between the baseline and maximum response after a specified exposure time is referred to as the “half maximal effective concentration” or “EC50”. The EC50 value is generally presented as a molar (moles/liter) amount, but it is often converted into a logarithmic value as follows −log₁₀(EC50). For example, if the EC50 equals 1 μM (10⁻⁶ M), the log₁₀(EC50) value is −6. Another value used is pEC50, which is defined as the negative logarithm of the EC50 (−log₁₀(EC50)). In the above example, the EC50 equaling 1 μM has a pEC50 value of 6. In certain embodiments, the functional avidity of a binding protein of this disclosure will comprise a measure of an ability of the binding protein to promote activation and/or IFNγ production by T cells, which can be measured using assays known in the art and described herein. In certain embodiments, functional avidity will comprise a measure of the ability of the binding protein, upon binding to antigen, to activate a host cell, such as a T cell.

Binding proteins disclosed herein can comprise high functional avidity that can, for example, facilitate elicitation of immune cell effector functions (e.g., activation, proliferation, cytokine production, and/or cytotoxicity) against even lower levels of a presented a neoantigen peptide, such as the KRAS G12V mutant peptide of SEQ ID NO: 2 or SEQ ID NO: 3.

In some embodiments, the binding protein has a log 10EC50 for the neoantigen peptide of about −6.0 or less, about −6.1 or less, about −6.2 or less, about −6.3 or less, about −6.4 or less, about −6.5 or less, about −6.6 or less, about −6.7 or less, about −6.8 or less, about −6.9 or less, about −7.0 or less, about −7.1 or less, about −7.2 or less, about −7.3 or less, about −7.4 or less, about −7.5 or less, about −7.6 or less, about −7.7 or less, about −7.8 or less, about −7.9 or less, about −8.0 or less, about −8.1 or less, about −8.2 or less, about −8.3 or less, about −8.4 or less, about −8.5 or less, about −8.6 or less, about −8.7 or less, about −8.8 or less, about −8.9 or less, about −9 or less, about −9.1 or less, about −9.2 or less, about −9.3 or less, about −9.4 or less, about −9.5 or less, about −9.6 or less, about −9.7 or less, about −9.8 or less, about −9.9 or less, or about −10 or less.

In some embodiments, a host cell disclosed herein comprises a binding protein (e.g., TCR) that binds a target neoantigen of the binding protein (for example, a KRAS G12 mutant peptide, such as KRAS G12V mutant peptide, e.g., present in a peptide:HLA complex) with an EC50 (e.g., peptide dose at which a half-maximal activation of a T cell population is reached) of less than about 100 mM, less than about 10 mM, less than about 1 mM, less than about 500 M, less than about 100 μM, less than about 50 μM, less than about 10 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 900 μM, less than about 800 μM, less than about 700 μM, less than about 600 μM, less than about 500 μM, less than about 400 μM, less than about 300 μM, less than about 200 μM, less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, or less than about 1 μM. The EC50 can be determined by an assay to identify a peptide dose at which a half-maximal activation of a T cell population is reached, e.g., as reflected by expression an activation marker (e.g., CD137, CD69, Granzyme B, CD107a, IFN-gamma, TNF-α, IL-12, a cytokine, an interleukin, an interferon) upon exposure to target cells in the presence of various concentrations of the mutant peptide.

In some embodiments, a host cell disclosed herein comprises a binding protein (e.g., TCR) that binds a target neoantigen of the binding protein (for example, a KRAS G12 mutant peptide, such as KRAS G12V mutant peptide, e.g., present in a peptide:HLA complex) with an EC50 (e.g., peptide dose at which a half-maximal activation of a T cell population is reached) of at least about 100 mM, at least about 10 mM, at least about 1 mM, at least about 500 μM, at least about 100 μM, at least about 50 μM, at least about 10 μM, at least about 5 μM, at least about 4 μM, at least about 3 μM, at least about 2 μM, at least about 1 μM, at least about 900 nM, at least about 800 nM, at least about 700 nM, at least about 600 nM, at least about 500 nM, at least about 400 nM, at least about 300 nM, at least about 200 nM, at least about 100 nM, at least about 90 nM, at least about 80 nM, at least about 70 nM, at least about 60 nM, at least about 50 nM, at least about 40 nM, at least about 30 nM, at least about 20 nM, at least about 10 nM, at least about 5 nM, at least about 1 nM, at least about 900 pM, at least about 800 pM, at least about 700 pM, at least about 600 pM, at least about 500 pM, at least about 400 pM, at least about 300 pM, at least about 200 pM, at least about 100 pM, at least about 90 pM, at least about 80 pM, at least about 70 pM, at least about 60 pM, at least about 50 pM, at least about 40 pM, at least about 30 pM, at least about 20 pM, at least about 10 pM, at least about 5 pM, or at least about 1 pM.

A host cell can comprise a transgenic polynucleotide encoding a chimeric fusion protein that comprises an IL7R intracellular signaling domain. The chimeric fusion protein can comprise, for example, an intracellular portion of an Interleukin 7 Receptor A (IL7RA) polypeptide, or a portion or variant thereof that is capable of contributing to an IL-7 signal in a host cell. A chimeric IL7R fusion protein can, for example, provide a “signal 3” to increase STAT5 phosphorylation and host cell functionality, enhance proliferation of a host cell, increase host cell survival (e.g., in the tumor microenvironment), and/or enhance chemokine receptor expression.

Interleukin-7 receptor subunit alpha can also be referred to as IL7R-α, as IL7RA, as IL-7R-alpha, as ILRA, as Interleukin-7 receptor-α, as interleukin 7 receptor, as Cluster of Differentiation 127 as CD127, or as CDW127.

An IL7R intracellular signaling domain can comprise an amino acid sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to SEQ ID NO: 1041.

In some embodiments, the IL7R intracellular signaling domain comprises (a) one or more residues of a BOX1 motif corresponding to residues 8-15 (VWPSLPDH) relative to SEQ ID NO: 1041 when optimally aligned, or (b) Y185 relative to SEQ ID NO: 1041 when optimally aligned. In some embodiments, the IL7R intracellular signaling domain comprises one or more residues of a FERM domain corresponding to residues 1-6 (KKRIKPI) or residues 16-28 (KKTLEHLCKKPRK) relative to SEQ ID NO: 1041 when optimally aligned.

In some embodiments, the chimeric fusion protein comprises an IL7R transmembrane domain. The IL7R transmembrane domain can comprise an amino acid sequence with at least 80%, at least 85%, at least 90%, at least 95%, at least 99%, or 100% sequence identity to SEQ ID NO: 1042. In some embodiments, the IL7R transmembrane domain comprises a mutation relative to SEQ ID NO: 1042. In some embodiments, the mutation is, or comprises, the insertion of one or more cysteines, and/or one or more prolines, into the amino acid sequence of SEQ ID NO: 1042. In some embodiments, the mutation enables or facilitates homodimerization of the receptor. In some embodiments, the mutation comprises an insertion of a trimer peptide of cysteine, proline, threonine (CPT) into the transmembrane domain. In some embodiments, the threonine of the CPT insertion is not threonine but another amino acid, and in at least specific cases that other amino acid is or is not cysteine or proline. In some embodiments, the chimeric fusion protein comprises a transmembrane domain of IL7R, IL2RA, IL2RB, IL2RG, IL14R, IL15R, IL9R, IL21R, CD2, CD40L, CD58, CD80, or SIRPα.

In some embodiments, the chimeric fusion protein comprises an extracellular component comprising: (i) an extracellular domain of a Cluster of Differentiation 80 (CD80) polypeptide, or a portion or variant thereof that is capable of binding a CD28 or CTLA-4 polypeptide; (ii) an extracellular domain of a Cluster of Differentiation 58 (CD58) polypeptide, or a portion or variant thereof that is capable of binding a Cluster of Differentiation 2 (CD2) polypeptide; (iii) an extracellular domain of a Signal Regulatory Protein Alpha (SIRPα) polypeptide, or a portion or variant thereof that is capable of binding a Cluster of Differentiation 47 (CD47) polypeptide; (iv) an extracellular domain of a Cluster of Differentiation 40L (CD40L) polypeptide, or a portion or variant thereof that is capable of binding a CD40 polypeptide; (v) an extracellular domain of a Cluster of Differentiation 2 (CD2) receptor, or a portion or variant thereof that is capable of binding a CD58 polypeptide; or (vi) an extracellular domain of a Cluster of Differentiation 34 (CD34) polypeptide.

In some embodiments, the chimeric fusion protein comprises an extracellular component comprising an extracellular domain of a Cluster of Differentiation 80 (CD80) polypeptide, or a portion or variant thereof that is capable of binding a CD28 or CTLA-4 polypeptide. In some embodiments, the extracellular domain of CD80 comprises an amino acid sequence with at least 80%, at least 85%, at least 90%, at least 95%, at least 98%, at least 99%, or 100% sequence identity to SEQ ID NO: 1043.

In some embodiments, the chimeric fusion protein comprises an extracellular component comprising an extracellular domain of a Cluster of Differentiation 58 (CD58) polypeptide, or a portion or variant thereof that is capable of binding a CD28 or CTLA-4 polypeptide. In some embodiments, the extracellular domain of CD8β comprises an amino acid sequence with at least 80%, at least 85%, at least 90%, at least 95%, at least 98%, at least 99%, or 100% sequence identity to SEQ ID NO: 1044.

In some embodiments, the chimeric fusion protein comprises an extracellular component comprising an extracellular domain of CD34. In some embodiments, the extracellular domain of CD34 comprises an amino acid sequence with at least 80%, at least 85%, at least 90%, at least 95%, at least 98%, at least 99%, or 100% sequence identity to SEQ ID NO: 1045.

In some embodiments, a population of host cells comprising one or more modifications disclosed herein (e.g., expression of a Fas-41BB fusion protein or chimeric IL7R polypeptide disclosed herein) exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 10 fold, at least 50 fold, or at least 100 fold, at least 500 fold, or at least 1000 fold increased proliferation in response to target cells (e.g., that present a KRAS G12D peptide) as compared to a population of control cells (for example, corresponding cells lacking the Fas-41BB fusion protein or chimeric IL7R polypeptide). The proliferation can be, for example, as determined by an in vitro lymphoproliferation assay or measurement of host cell numbers after co-incubation. The host cells can comprise an extracellular binding protein (e.g., a TCR comprising Vα and Vβ regions and/or CDRs disclosed herein), and/or a modification that results in decreased expression of endogenous TRAC, TRBC1, and/or TRBC2.

In some embodiments, a population of host cells comprising one or more modifications disclosed herein (e.g., expression of a Fas-41BB fusion protein or chimeric IL7R polypeptide disclosed herein) exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 10 fold, at least 50 fold, or at least 100 fold, at least 500 fold, or at least 1000 fold increased killing of target cells as compared to a population of control cells (for example, corresponding cells lacking the Fas-41BB fusion protein or chimeric IL7R polypeptide). The killing of target cells can be, for example, as determined by an in vitro cytotoxicity assay. The host cells can comprise an extracellular binding protein (e.g., a TCR comprising Vα and Vβ regions and/or CDRs disclosed herein), and/or a modification that results in decreased expression of endogenous TRAC, TRBC1, and/or TRBC2.

A nucleic acid encoding a polypeptide disclosed herein (e.g., extracellular binding protein, CD8 co-receptor chain or an extracellular portion thereof, Fas-41BB fusion protein, or chimeric IL7R fusion protein) can encode a signal peptide. In some cases, a polypeptide of the disclosure comprises a signal peptide. A signal peptide can be cleaved off during processing of the polypeptide, thus in some cases a mature polypeptide disclosed herein does not contain a signal peptide.

A signal peptide at the N-terminus of a protein can be involved in transport of the protein to or through a membrane, transport to different a membranous cellular compartment, or secretion of the protein from the cell. A nucleic acid encoding a protein of the disclosure can encode a signal peptide to facilitate membrane insertion and surface localization of the protein. A signal peptide can be selected for its ability to facilitate ER processing and cell surface localization of the protein. Any suitable signal peptide can be used. In some cases, the signal peptide can comprise a G-CSF signal peptide or a CD8α signal peptide. A signal peptide can be about 10 to about 40 amino acids in length. In some cases, a signal peptide is at least about 10, 15, 16, 20, 21, 22, 25, or 30 amino acids in length, or more. In some cases, a signal peptide is at most about 15, 16, 20, 21, 22, 25, or 30 amino acids in length, or less. In some cases, a signal peptide is about 16-30 amino acids in length.

In some embodiments, a binding protein (e.g., TCR) binds a target (for example, a KRAS G12 mutant peptide, such as KRAS G12V mutant peptide, e.g., present in a peptide:HLA complex) with a KD of less than about 100 mM, less than about 10 mM, less than about 1 mM, less than about 500 μM, less than about 100 μM, less than about 50 μM, less than about 10 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 900 pM, less than about 800 pM, less than about 700 pM, less than about 600 pM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, or less than about 1 pM.

Also contemplated are fusion proteins comprising a scTCR or scTv of the present disclosure linked to the constant domain of an antibody (e.g., IgG (1, 2, 3, 4), IgE, IgD, IgA, IgM, and variants thereof) or a fragment thereof (e.g., a fragment that, in some embodiments, retains binding to one or more Fc receptors, to C1q, to Protein A, to Protein G, or any combination thereof), and including immunoglobulin heavy chain monomers and multimers, such as Fc dimers; see, e.g., Wong et al., J. Immunol. 198:1 Supp. (2017). Variant Fc polypeptides comprising mutations that enhance, reduce, or abrogate binding to or by, e.g., FcRn or other Fc receptors, are known and are contemplated within this disclosure.

In certain embodiments, a binding protein or fusion protein (e.g., TCR, scTCR, CAR) of the present disclosure is expressed by a host cell (e.g., by a T cell, NK cell, or NK-T cell heterologously expressing the binding protein or fusion protein). Avidity of such a host cell for a neoantigen (e.g., KRAS (or NRAS, or HRAS), p53, and/or PIK3CA) peptide antigen or a neoantigen (e.g., KRAS (or NRAS, or HRAS), p53, and/or PIK3CA) peptide antigen:HLA complex can be determined by, for example, exposing the host cell to the peptide, or to a peptide:HLA complex (e.g., organized as a tetramer), or to an antigen-presenting cell (APC) that presents the peptide to the host cell, optionally in a peptide:HLA complex, and then measuring an activity of the host cell, such as, for example, production or secretion of cytokines (e.g., IFN-γ; TNFα); increased expression of host cell signaling or activation components (e.g., CD137 (4-1BB)); proliferation of the host cell; or killing of the APC (e.g., using a labeled-chromium release assay).

As used herein, “nucleic acid” or “nucleic acid molecule” or “polynucleotide” generally refers to any of deoxyribonucleic acid (DNA), ribonucleic acid (RNA), oligonucleotides, polynucleotides, fragments thereof generated, for example, by the polymerase chain reaction (PCR) or by in vitro translation, and also to fragments generated by any of ligation, scission, endonuclease action, or exonuclease action. In certain embodiments, the nucleic acids of the present disclosure are produced by PCR. Nucleic acids can be composed of monomers that are naturally occurring nucleotides (such as deoxyribonucleotides and ribonucleotides), analogs of naturally occurring nucleotides (e.g., α-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single-stranded or double-stranded.

The term “isolated” generally means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid can be part of a vector and/or such nucleic acid or polypeptide can be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

As used herein, the terms “recombinant”, “engineered”, and “modified” generally refer to a cell, microorganism, nucleic acid molecule, polypeptide, protein, plasmid, or vector that has been modified by introduction of an exogenous nucleic acid molecule, or refers to a cell or microorganism that has been genetically engineered by human intervention—that is, modified by introduction of a heterologous nucleic acid molecule, or refers to a cell or microorganism that has been altered such that expression of an endogenous nucleic acid molecule or gene is controlled, deregulated or constitutive, where such alterations or modifications can be introduced by genetic engineering. Human-generated genetic alterations can include, for example, modifications introducing nucleic acid molecules (which may include an expression control element, such as a promoter) encoding one or more proteins or enzymes, or other nucleic acid molecule additions, deletions, substitutions, or other functional disruption of or addition to a cell's genetic material. Example modifications include those in coding regions or functional fragments thereof of heterologous or homologous polypeptides from a reference or parent molecule.

As used herein, “mutation” generally refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). In certain embodiments, a mutation is a substitution of one or three codons or amino acids, a deletion of one to about 5 codons or amino acids, or a combination thereof.

A “conservative substitution” generally refers to a substitution of one amino acid for another amino acid that has similar properties. Example conservative substitutions are well known in the art (see, e.g., WO 97/09433 at page 10; Lehninger, Biochemistry, 2^{nd} Edition; Worth Publishers, Inc. NY, NY, pp. 71-77, 1975; Lewin, Genes IV, Oxford University Press, NY and Cell Press, Cambridge, MA, p. 8, 1990).

In certain embodiments, proteins (e.g., binding protein, immunogenic peptide) according to the present disclosure comprise a variant sequence as compared to a reference sequence (e.g., a variant TCR CDR (e.g., CDR3β_as compared to a reference TCR CDR3β disclosed herein). As used herein, a “variant” amino acid sequence, peptide, or polypeptide, refers to an amino acid sequence (or peptide or polypeptide) having one, two, or three amino acid substitutions, deletions, and/or insertions as compared to a reference amino acid sequence. In certain embodiments, a variant amino acid sequence, peptide, or polypeptide, retains substantially a same functionality (e.g., binding specificity and affinity for a peptide:HLA complex) as the reference molecule; for example, a variant TCR fragment as disclosed herein retains about 50%, about 60%, about 70%, about 80%, about 90%, about 95%, about 99%, at least about 50%, at least about 60%, at least about 70%, at least about 80%, at least about 90%, at least about 95%, at least about 99%, or 100% of the antigen-binding specificity and affinity as compared to a reference TCR binding fragment.

An“altered domain” or “altered protein” generally refers to a motif, region, domain, peptide, polypeptide, or protein with a non-identical sequence identity to a wild type motif, region, domain, peptide, polypeptide, or protein (e.g., a wild type TCRα chain, TCRβ chain, TCRα constant domain, TCRβ constant domain) of at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9%).

Altered domains or altered proteins or derivatives can include those based on all possible codon choices for the same amino acid and codon choices based on conservative amino acid substitutions. For example, the following six groups each contain amino acids that are conservative substitutions for one another: 1) alanine (ala; A), serine (ser; S), threonine (thr; T); 2) aspartic acid (asp; D), glutamic acid (glu; E); 3) asparagine (asn; N), glutamine (gln; Q); 4) arginine (arg; R), lysine (lys; K); 5) Isoleucine (ile; I), leucine (L), methionine (met; M), valine (val; V); and 6) phenylalanine (phe; F), tyrosine (tyr; Y), tryptophan (trp; W). (See also WO97/09433 at page 10, Lehninger, Biochemistry, 2^{nd} Edition, Worth Publishers, Inc., NY, NY, pp. 71-77, 1975; Lewin Genes IV, Oxford University Press, NY and Cell Press, Cambridge, MA, p.8, 1990; Creighton, Proteins, W. H. Freeman and Company 1984). In addition, individual substitutions, deletions or additions that alter, add or delete, a single amino acid or a small percentage of amino acids in an encoded sequence are also “conservative substitutions.”

The term “construct” generally refers to any polynucleotide that contains a recombinant nucleic acid molecule. A “transgene” or “transgene construct” refers to a construct that contains two or more genes operably linked in an arrangement that is not found in nature. The term “operably-linked” (or “operably linked” herein) generally refers to the association of two or more nucleic acid molecules on a single nucleic acid fragment so that the function of one is affected by the other. For example, a promoter is operably-linked with a coding sequence when it can affect the expression of that coding sequence (i.e., the coding sequence is under the transcriptional control of the promoter). “Unlinked” generally means that the associated genetic elements are not closely associated with one another and the function of one does not affect the other. In some embodiments, the genes present in a transgene are operably linked to an expression control sequence (e.g., a promoter).

A construct (e.g., a transgene) can be present in a vector (e.g., a bacterial vector, a viral vector) or can be integrated into a genome. A “vector” generally refers to a nucleic acid molecule that is capable of transporting another nucleic acid molecule. Vectors can be, for example, plasmids, cosmids, viruses, a RNA vector or a linear or circular DNA or RNA molecule that can include chromosomal, non-chromosomal, semi-synthetic or synthetic nucleic acid molecules. Example vectors are those capable of autonomous replication (episomal vector) or expression of nucleic acid molecules to which they are linked (expression vectors). Vectors useful in the compositions and methods of this disclosure are described further herein.

The term “expression,” as used herein, generally refers to the process by which a polypeptide is produced based on the encoding sequence of a nucleic acid molecule, such as a gene. The process can include transcription, post-transcriptional control, post-transcriptional modification, translation, post-translational control, post translational modification, or any combination thereof.

The term “introduced” in the context of inserting a nucleic acid molecule into a cell, generally means “transfection,” or “transformation,” or “transduction” and includes reference to the incorporation of a nucleic acid molecule into a eukaryotic or prokaryotic cell wherein the nucleic acid molecule can be incorporated into the genome of a cell (e.g., a chromosome, a plasmid, a plastid, or a mitochondrial DNA), converted into an autonomous replicon, or transiently expressed (e.g., transfected mRNA).

As used herein, “heterologous” or “exogenous” nucleic acid molecule, construct, or sequence generally refers to a nucleic acid molecule or portion of a nucleic acid molecule that is not native to a host cell but can be homologous to a nucleic acid molecule or portion of a nucleic acid molecule from the host cell. The source of the heterologous or exogenous nucleic acid molecule, construct or sequence can be from a different genus or species. In certain embodiments, a heterologous or exogenous nucleic acid molecule is added (i.e., not endogenous or native) to a host cell or host genome by, for example, conjugation, transformation, transfection, transduction, electroporation, or the like, wherein the added molecule can integrate into the host genome or exist as extra-chromosomal genetic material (e.g., as a plasmid or other form of self-replicating vector), and can be present in multiple copies. In addition, “heterologous” refers to a non-native enzyme, protein or other activity encoded by an exogenous nucleic acid molecule introduced into the host cell, even if the host cell encodes a homologous protein or activity. Moreover, a cell comprising a “modification” or a “heterologous” polynucleotide or binding protein includes progeny of that cell, regardless of whether the progeny were themselves transduced, transfected, or otherwise manipulated or changed.

As described herein, more than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express one or more heterologous or exogenous nucleic acid molecule encoding desired TCR specific for a Ras antigen peptide (e.g., TCRα and TCRβ) and optionally, as disclosed herein, also encoding a CD8 co-receptor polypeptide comprising a α chain, a β chain, or a portion thereof, such as an extracellular portion capable of binding to MHC. When two or more exogenous nucleic acid molecules are introduced into a host cell, it is understood that the two or more exogenous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “endogenous” or “native” generally refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., a promoter, translational attenuation sequences) can be used to alter or regulate expression of a second native gene or nucleic acid molecule, wherein the expression or regulation of the second native gene or nucleic acid molecule differs from normal expression or regulation in a parent cell.

The term “homologous” or “homolog” generally refers to a molecule or activity found in or derived from a host cell, species or strain. For example, a heterologous or exogenous nucleic acid molecule can be homologous to a native host cell gene, and can optionally have an altered expression level, a different sequence, an altered activity, or any combination thereof.

“Sequence identity,” as used herein, generally refers to the percentage of amino acid residues or nucleobases in one sequence that are identical with the amino acid residues or nucleobases (respectively) in a reference sequence after aligning the sequences and introducing gaps, if necessary, to achieve the maximum percent sequence identity, and not considering any conservative substitutions as part of the sequence identity. The percentage sequence identity values can be generated using the NCBI BLAST 2.0 software as defined by Altschul et al. (1997), Nucl. Acids Res. 25:3389-3402, with the parameters set to default values. Additionally or alternatively, the degree of sequence identity between two sequences can be determined, for example, by comparing the two sequences using computer programs designed for this purpose, such as global or local alignment algorithms. Non-limiting examples include BLASTp, BLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm. A global alignment algorithm, such as a Needleman and Wunsch algorithm, can be used to align two sequences over their entire length, maximizing the number of matches and minimizing the number of gaps. Default settings can be used.

To generate similarity scores for two amino acid sequences, scoring matrices can be used that assign positive scores for some non-identical amino acids (e.g., conservative amino acid substitutions, amino acids with similar physio-chemical properties, and/or amino acids that exhibit frequent substitutions in orthologs, homologs, or paralogs), Non-limiting examples of scoring matrices include PAM30, PAM70, PAM250, BLOSUM45, BLOSUM50, BLOUM62, BLOSUM80, and BLOSUM90.

Variants of nucleic acid molecules of this disclosure are also contemplated. Variant nucleic acid molecules are at least 70%, at least 75%, at least 80%, at least 85%, at least 90%, and can be at least 95%, at least 96%, at least 97%, at least 98%, at least 99%, or at least 99.9% identical to a nucleic acid molecule of a defined or reference polynucleotide as described herein, or that hybridize to a polynucleotide under stringent hybridization conditions of 0.015 M sodium chloride, 0.0015 M sodium citrate at about 65-68° C. or 0.015 M sodium chloride, 0.0015 M sodium citrate, and 50% formamide at about 42° C. Nucleic acid molecule variants retain the capacity to encode a binding protein or a binding domain thereof having a functionality described herein, such as binding a target molecule.

The term “isolated” generally means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid can be part of a vector and/or such nucleic acid or polypeptide can be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

In some contexts, the term “variant” as used herein, generally refers to at least one fragment of the full-length sequence referred to, more specifically one or more amino acid or nucleic acid sequence which is, relative to the full-length sequence, truncated at one or both termini by one or more amino acids. Such a fragment includes or encodes for a peptide having at least 6, 7, 8, 10, 12, 15, 20, 25, 50, 75, 100, 150, or 200 successive amino acids of the original sequence or a variant thereof. The total length of the variant may be at least 6, 7, 8, 9, 10, 11, 12, 20, 25, 30, 40, 50, 60, 70, 80, 90, 100, or more amino acids.

In some embodiments, the term “variant” relates not only to at least one fragment, but also to a polypeptide or a fragment thereof including amino acid sequences that are at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to the reference amino acid sequence referred to or the fragment thereof, wherein amino acids other than those essential for the biological activity or the fold or structure of the polypeptide are deleted or substituted, one or more such essential amino acids are replaced in a conservative manner, and/or amino acids are added such that the biological activity of the polypeptide is preserved. The state of the art includes various methods that may be used to align two given nucleic acid or amino acid sequences and to calculate the degree of identity (see, e.g., Arthur Lesk (2008), Introduction to bioinformatics, Oxford University Press, 2008, 3rd edition). In some embodiments, the Clustal W software can be used using default settings (Larkin, M. A., et al. (2007). Clustal W and Clustal X version 2.0. Bioinformatics, 23, 2947-2948).

In certain embodiments, variants may, in addition, include chemical modifications, for example, isotopic labels or covalent modifications such as glycosylation, phosphorylation, acetylation, decarboxylation, citrullination, hydroxylation and the like. Methods for modifying polypeptides are known and in general will be employed so as not to abolish or substantially diminish a desired activity of the polypeptide.

In an embodiment, the term “variant” of a nucleic acid molecule includes nucleic acids the complementary strand of which hybridizes, for example, under stringent conditions, to the reference or wild type nucleic acid. Stringency of hybridization reactions is readily determinable by one of ordinary skill in the art, and in general is an empirical calculation dependent on probe length, washing temperature, and salt concentration. In general, longer probes require higher temperatures for proper annealing, while shorter probes less so. Hybridization generally depends on the ability of denatured DNA to reanneal to complementary strands present in an environment below their melting temperature: the higher the degree of desired homology between the probe and hybridizable sequence, the higher the relative temperature which may be used. As a result, higher relative temperatures can make the reaction conditions more stringent, while lower temperatures less so. For additional details and explanation of stringency of hybridization reactions, see Ausubel, F. M. (1995), Current Protocols in Molecular Biology. John Wiley & Sons, Inc. Moreover, the person skilled in the art may follow the instructions given in the manual Boehringer Mannheim GmbH (1993) The DIG System Users Guide for Filter Hybridization, Boehringer Mannheim GmbH, Mannheim, Germany and in Liebl, W., Ehrmann, M., Ludwig, W., and Schleifer, K. H. (1991) International Journal of Systematic Bacteriology 41: 255-260 on how to identify DNA sequences by means of hybridization. In an embodiment, stringent conditions are applied for any hybridization, i.e., hybridization occurs only if the probe is 70% or more identical to the target sequence. Probes having a lower degree of identity with respect to the target sequence may hybridize, but such hybrids are unstable and will be removed in a washing step under stringent conditions, for example, lowering the concentration of salt to 2×SSC or, optionally and subsequently, to 0.5×SSC, while the temperature is, for example, about 50° C.-68° C., about 52° C.-68° C., about 54° C.-68° C., about 56° C.-68° C., about 58° C.-68° C., about 60° C.-68° C., about 62° C.-68° C., about 64° C.-68° C., or about 66° C.-68° C. In an embodiment, the temperature is about 64° C.-68° C. or about 66° C.-68° C. It is possible to adjust the concentration of salt to 0.2×SSC or even 0.1×SSC. Nucleic acid sequences having a degree of identity with respect to the reference or wild type sequence of at least 70%, at least 80%, at least 85%, at least 90%, at least 91%, at least 92%, at least 93%, at least 94%, at least 95%, at least 96%, at least 97%, at least 98%, at least 99%, or at least 99.5% may be isolated. In an embodiment, the term variant of a nucleic acid sequence, as used herein, refers to any nucleic acid sequence that encodes the same amino acid sequence and variants thereof as the reference nucleic acid sequence, in line with the degeneracy of the genetic code.

A “functional variant” generally refers to a polypeptide or polynucleotide that is structurally similar or substantially structurally similar to a parent or reference compound of this disclosure, but differs, in some contexts slightly, in composition (e.g., one base, atom or functional group is different, added, or removed; or one or more amino acids are mutated, inserted, or deleted), such that the polypeptide or encoded polypeptide is capable of performing at least one function of the encoded parent polypeptide with at least 50% efficiency, or at least 55%, at least 60%, at least 70%, at least 75%, at least 80%, at least 85%, at least 90%, at least 95%, at least 96%, at least 97%, at least 98%, at least 99%, at least 99.9%, or at least 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (KD) constant), avidity, or activation of a host cell. As used herein, a “functional portion” or “functional fragment” refers to a polypeptide or polynucleotide that comprises only a domain, motif, portion or fragment of a parent or reference compound, and the polypeptide or encoded polypeptide retains at least 50% activity associated with the domain, portion or fragment of the parent or reference compound, or at least 55 at least 60%, at least 70%, at least 75%, at least 80%, at least 85%, at least 90%, at least 95%, at least 96%, at least 97%, at least 98%, at least 99%, at least 99.9%, or at least 100% level of activity of the parent polypeptide, or provides a biological benefit (e.g., effector function).

A “functional portion” or “functional fragment” of a polypeptide or encoded polypeptide of this disclosure generally has “similar binding” or “similar activity” when the functional portion or fragment displays no more than a 50% reduction in performance in a selected assay as compared to the parent or reference polypeptide (alternatively or additionally, no more than 20% or 10%, or no more than a log difference as compared to the parent or reference with regard to affinity), such as an assay for measuring binding affinity or measuring effector function (e.g., cytokine release). Functional variants of specifically disclosed binding proteins and polynucleotides are contemplated.

An “altered domain” or “altered protein” generally refers to a motif, region, domain, peptide, polypeptide, or protein with a non-identical sequence identity to a wild type motif, region, domain, peptide, polypeptide, or protein (e.g., a wild type TCRα chain, TCRβ chain, TCRα constant domain, or TCRβ constant domain) of at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9%).

Included in the current disclosure are variants of any of the binding proteins described herein (e.g., a TCR α-chain or a TCR β-chain, or fragments thereof such as Vα or Vβ chains or CDR1α, CDR2α, CDR3α, CDR1β, CDR2β, or CDR3β) with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally or alternatively, by comparing aligned sequences of homologous proteins from different species, conservative substitutions can be identified by locating amino acid residues that have been mutated between species (e.g., non-conserved residues without altering the basic functions of the encoded proteins. Such conservatively substituted variants may include variants with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity any one of the systems described herein. In some embodiments, such conservatively substituted variants are functional variants

Conservative substitution tables providing functionally similar amino acids are available from a variety of references (see, for example, Creighton, Proteins: Structures and Molecular Properties (W H Freeman & Co.; 2nd Edition (December 1993))). The following eight groups each contain amino acids that are conservative substitutions for one another:
a. Alanine (A), Glycine (G);
b. Aspartic acid (D), Glutamic acid (E);
c. Asparagine (N), Glutamine (Q);
d. Arginine (R), Lysine (K);
e. Isoleucine (I), Leucine (L), Methionine (M), Valine (V);
f. Phenylalanine (F), Tyrosine (Y), Tryptophan (W);
g. Serine (S), Threonine (T); and
h. Cysteine (C), Methionine (M).

### Binding Proteins

In one aspect, the present disclosure provides a binding protein, comprising a T cell receptor (TCR) α chain variable (Vα) domain and a TCR β chain variable (Vβ) domain, wherein the binding protein is capable of binding to a peptide:HLA complex, wherein the peptide comprises, consists essentially of, or consists of the amino acid sequence set forth in SEQ ID NO:2 or SEQ ID NO:3. In certain embodiments, the HLA comprises an HLA-A*11, optionally HLA-A*11:01. In any of the presently disclosed embodiments, the binding protein can be heterologously expressed by a human immune system cell, such as, for example, a T cell.

In certain embodiments, the Vα domain and/or the Vβ domain are each independently human, humanized, or chimeric, and each can be human. In some embodiments, the Vα domain is human and the Vβ domain is human. Binding proteins, compositions, and methods disclosed herein can utilize a Vα domain, Vβ domain, or CDRs therefrom derived from a human subject, for example, from sequencing of an isolated T cell or population thereof from a human subject. TCR Vα domains, Vβ domains, and CDRs therefrom isolated from a human subject can have advantageous properties over variable domains and CDRs from other sources, such as mice transgenic for a single human HLA allele. For example, Vα domains, Vβ domains, and CDRs derived from a human subject can have undergone negative thymic selection against substantially the whole human peptidome presented by a full set of human HLA molecules in vivo, which can reduce the likelihood that the binding protein is cross-reactive to other human self-antigens. In some embodiments, a binding protein disclosed herein is substantially non-reactive to a human proteome presented by one or more HLA alleles. The reactivity can be determined by any suitable method. In some embodiments, no significant response by binding protein-transduced T cells to the human proteome presented by the one or more HLA allele(s) is observed or predicted with peptide concentrations of 500 nM or lower, 400 nM or lower, 300 nM or lower, 200 nM or lower, 100 nM or lower, 50 nM or lower, 10 nM or lower, 5 nM or lower, or 1 nM or lower.

In some embodiments, a binding protein comprises one or more variable domains or one or more CDRs derived from (e.g., identified in) a T cell of a subject (e.g., a human subject) having a disease, such as a cancer. In some embodiments, a binding protein comprises one or more variable domains or one or more CDRs derived from a T cell of a human subject having a cancer disclosed herein. In some embodiments, a binding protein comprises one or more variable domains or one or more CDRs derived from a T cell of a subject (e.g., a human subject) having a disease associated with a neoantigen (e.g., KRAS (or NRAS, or HRAS), p53, and/or PIK3CA)mutation, such as a KRAS G12V or G12D mutation. In some embodiments, a binding protein comprises one or more variable domains or one or more CDRs derived from a T cell of a subject (e.g., a human subject) with a cell that comprises a neoantigen (e.g., KRAS (or NRAS, or HRAS), p53, and/or PIK3CA) mutation, such as a KRAS G12V or G12D mutation.

In some embodiments, a binding protein comprises one or more variable domains or one or more CDRs derived from a T cell of a healthy subject (e.g., a healthy human subject). In some embodiments, a healthy subject lacks a specific pathological diagnosis (e.g., disease diagnosis, such as a cancer diagnosis). In some embodiments, a healthy subject lacks a specific pathological diagnosis, but comprises a different pathological diagnosis, for example, lacks a cancer diagnosis but comprises a diagnosis of hypertension or type II diabetes.

Presently disclosed binding proteins are capable of being heterologously expressed by host cells, such as, for example, human immune cells, such as T cells. Furthermore, expression of a presently disclosed binding protein can confer advantageous properties upon a host cell; e.g., having binding specificity for a neoantigen:HLA complex of the present disclosure, improved activation, proliferation, or killing activity in the presence of a neoantigen:HLA presenting tumor cell, or the like.

For example, in certain embodiments, when the binding protein is expressed by an immune cell (e.g., a human T cell, optionally a CD8+ and/or CD4+ T cell, a NK cell, or a NK-T cell), the immune cell is capable of specifically killing a HLA-A*11:01⁺ tumor cell that expresses a peptide comprising or consisting of the amino acid sequence set forth in SEQ ID NO:2 or 3. Killing of a target cell can be determined, for example, the Incucyte® bioimaging platform (Essen Bioscience). In certain embodiments, this platform uses activated caspase and labelled (e.g., RapidRed or NucRed) tumor cell signals, wherein overlap is measured and increased overlap area equals tumor cell death by apoptosis. Killing can also be determined using a 4-hour assay in which target cells are loaded with labeled chromium (⁵¹Cr), and ⁵¹Cr and the supernatant is measured following 4-hour co-incubation with an immune cell expressing a binding protein of the present disclosure. In certain embodiments, a killing assay can be performed using an effector:target cell ratio of 0.1:1, 0.5:1, 1:1, 2:1, 3:1, 4:1, 5:1, 6:1, 7:1, 8:1, 9:1, 10:1, 20:1, 25:1, 50:1, or 100:1, or the like.

In any of the presently disclosed embodiments, when the binding protein is expressed by an immune cell (e.g., a human T cell, optionally a CD8+ and/or CD4+ T cell, a NK cell, or a NK-T cell), the immune cell has elevated expression of Nur77 when in the presence of a tumor cell (e.g. an HLA-A11:01⁺ tumor cell) that expresses a neoantigen peptide (e.g., a peptide comprising or consisting of the amino acid sequence set forth in SEQ ID NO:2 or 3), optionally in the further presence of exogenous IFN-γ, wherein the Nur77 expression is elevated as compared to: (i) Nur77 expression by a reference immune cell (i.e., of the same cell type as, and otherwise phenotypically and/or genotypically at least substantially identical or functionally equivalent to, the immune cell expressing the binding protein) not expressing the binding protein, when the reference immune cell is in the presence of the tumor cell; and/or (ii) Nur77 expression by the immune cell expressing the binding protein when not in the presence of the tumor cell and/or when not in the presence of an antigen-presenting cell expressing a neoantigen peptide:HLA complex (e.g., wherein the peptide comprises, consists essentially of, or consists of the amino acid sequence set forth in SEQ ID NO:2 or 3, and wherein the HLA is optionally HLA-A*11:01). Expression of Nur77 can be determined, for example, using a transgenic expression construct comprising a Nur77 locus operably linked to a sequence encoding a reporter construct; e.g., dTomato (see Ahsouri and Weiss, J Immunol 198(2):657-668 (2017)).

In any of the presently disclosed embodiments, when the binding protein is expressed by an immune cell (e.g., a human T cell, optionally a CD8+ and/or CD4+ T cell, a NK cell, or a NK-T cell), the immune cell has elevated expression of CD137 (also known as 41BB) when in the presence of a HLA-A*02⁺ tumor cell that expresses a neoantigen peptide (e.g., a peptide comprising or consisting of the amino acid sequence set forth in SEQ ID NO:2 or 3), optionally in the further presence of exogenous IFN-γ, wherein the CD137 expression is elevated as compared to: (i) CD137 expression by a reference immune cell not expressing the binding protein, when the reference immune cell is in the presence of the tumor cell; and/or (ii) CD137 expression by the immune cell expressing the binding protein when not in the presence of the tumor cell and/or when not in the presence of an antigen-presenting cell expressing a neoantigen peptide:HLA complex (e.g., wherein the peptide comprises, consists essentially of, or consists of the amino acid sequence set forth in SEQ ID NO:2 or 3, and wherein the HLA is optionally HLA-A*11:01). CD137 expression can be determined using, for example, flow cytometry using a labeled anti-CD137 antibody. In certain embodiments, CD137 is measured following a 16-hour assay in which the immune cell is co-incubated with or stimulated with peptide or a target cell expressing the peptide.

In any of the presently disclosed embodiments: (i) the binding protein is encoded by a polynucleotide that is heterologous to the immune cell; (ii) the immune cell comprises a human CD8⁺ T cell, a human CD4+ T cell, or both; (iii) the tumor cell expressing a neoantigen peptide (e.g., a peptide comprising or consisting of the amino acid sequence set forth in SEQ ID NO:2 or 3 is HLA-A*11:01⁺); and/or (iv) the tumor cell comprises a OVCAR5 (ovarian serous adenocarcinoma), DAN-G (pancreatic adenocarcinoma), CFPAC1 (pancreatic adenocarcinoma), SW480 (colon carcinoma), SW527 (breast carcinoma), or NCI-H441 (lung adenocarcinoma) cell.

In certain embodiments, the binding protein is capable of binding to the peptide:HLA complex independent of, or in the absence of, CD8. CD8-independent binding can be determined by expressing the binding protein in a CD8-negative cell (e.g., a CD4⁺ T cell, a Jurkat cell, or the like) and identifying binding of the cell to a target. In some embodiments, a binding protein is provided that comprises: (a) a T cell receptor (TCR) α chain variable (Vα) domain comprising the complementarity determining region 3 (CDR3α) amino acid sequence set forth in any one of SEQ ID NOs:16, 17, 42, and 43, or a variant thereof having one, two, or three, optionally conservative, amino acid substitutions; and/or (b) a TCR β chain variable (Vβ) domain comprising the CDR3β amino acid sequence set forth in any one of SEQ ID NOs:26, 27, 52, and 53, or a variant thereof having one, two, or three, optionally conservative, amino acid substitutions, wherein the binding protein is capable of binding to a peptide:HLA complex, wherein the peptide comprises, consists essentially of, or consists of the amino acid sequence VVVGAVGVGK (SEQ ID NO:2) or VVGAVGVGK (SEQ ID NO:3) and wherein the HLA comprises an HLA-A*11. In certain embodiments, the HLA comprises HLA-A*11:01.

The Vα domain and/or the Vβ domain can be human, humanized, or chimeric, and can be human.

In certain embodiments, the binding protein comprises the CDR3α and CDR3β amino acid sequences set forth in SEQ ID NOs: (i) 17 and 27, respectively, or variants thereof having one, two, or three, optionally conservative, amino acid substitutions; (ii) 16 and 26, respectively, or variants thereof having one, two, or three, optionally conservative, amino acid substitutions; (iii) 53 and 43, respectively, or variants thereof having one, two, or three, optionally conservative, amino acid substitutions; or (iv) 52 and 42, respectively, or variants thereof having one, two, or three, optionally conservative, amino acid substitutions.

In some embodiments, the binding protein further comprises: (i) in the Vα domain, the CDR1α amino acid sequence set forth in SEQ ID NO: 14 or 40, or a variant thereof having one or two, optionally conservative, amino acid substitutions; (ii) in the Vα domain, the CDR2α amino acid sequence set forth in SEQ ID NO:15 or 41, or a variant thereof having one or two, optionally conservative, amino acid substitutions; (iii) in the Vβ domain, the CDR1β acid sequence set forth in SEQ ID NO:24 or 50, or a variant thereof having one or two, optionally conservative, amino acid substitutions; (iv) in the Vβ domain, the CDR2β acid sequence set forth in SEQ ID NO:25 or 51, or a variant thereof having one or two, optionally conservative, amino acid substitutions; or (v) any combination of (i)-(iv).

In certain embodiments, the binding protein comprises the CDR1α, CDR2α, CDR3α, CDR1β, CDR2β, and CDR3β amino acid sequences set forth in SEQ ID NOs: 14, 15, 16 or 17, 24, 25, and 26 or 27, respectively.

In other embodiments, the binding protein comprises the CDR1α, CDR2α, CDR3α, CDR1β, CDR2β, and CDR3β amino acid sequences set forth in SEQ ID NOs: 40, 41, 42 or 43, 50, 51, and 52 or 52, respectively.

In some embodiments: (i) the Vα domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO: 13 or 39; and/or (ii) the Vβ domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO:23 or 49.

In some embodiments, the Vα domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO:13, and wherein the Vβ domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO:23.

In some embodiments, the Vα domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO:39, and wherein the Vβ domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO:49.

In certain embodiments, the Vα domain comprises, consists essentially of, or consists of the amino acid sequence set forth in SEQ ID NO:13 and the and the Vβ domain comprises or consist of amino acid sequence set forth in SEQ ID NO:23.

In certain embodiments, the Vα domain comprises, consists essentially of, or consists of the amino acid sequence set forth in SEQ ID NO:39 and the and the Vβ domain comprises or consist of amino acid sequence set forth in SEQ ID NO:49.

In some embodiments, the variable domain comprises an amino acid sequence with one or more insertions, deletions, and/or substitutions relative to any one of SEQ ID NOs: 13, 23, 39, and 49.

For example, the variable domain can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 13, 23, 39, and 49.

In some embodiments, the variable domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 13, 23, 39, and 49.

In some embodiments, the variable domain comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 13, 23, 39, and 49.

The one or more insertions can be at the N-terminus, the C-terminus, within the amino acid sequence, or a combination thereof. The one or more insertions can be contiguous, non-contiguous, or a combination thereof.

In some embodiments, the variable domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 13, 23, 39, and 49.

In some embodiments, the variable domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 13, 23, 39, and 49.

In some embodiments, the variable domain comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 13, 23, 39, and 49.

The one or more deletions can be at the N-terminus, the C-terminus, within the amino acid sequence, or a combination thereof. The one or more deletions can be contiguous, non-contiguous, or a combination thereof.

In some embodiments, the variable domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 13, 23, 39, and 49.

In some embodiments, the variable domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 13, 23, 39, and 49.

In some embodiments, the variable domain comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 13, 23, 39, and 49.

The one or more substitutions can be at the N-terminus, the C-terminus, within the amino acid sequence, or a combination thereof. The one or more substitutions can be contiguous, non-contiguous, or a combination thereof.

The binding protein can further comprise a TCR α chain constant domain (Cα) and/or a TCR β chain constant domain (Cβ). The TCR α chain constant domain (Cα) and/or a TCR β chain constant domain (Cβ) can be human. The TCR α chain constant domain (Cα) and/or a TCR β chain constant domain (Cβ) can be mammalian. The TCR α chain constant domain (Cα) and/or a TCR β chain constant domain (Cβ) can be engineered.

In some embodiments, the Cα comprises, consists essentially of, or consists of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs:18, 19, 44, 45, and 69.

In some embodiments, the Cβ comprises, consists essentially of, or consists of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs: 28, 29, 54, 55, and 70-73.

In certain embodiments, the Cα and the Cβ comprise or consist of amino acid sequences having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the amino acid sequences set forth in SEQ ID NOs: (i) 18 and 28, respectively; (ii) 19 and 29, respectively; (iii) 44 and 54, respectively; or (iv) 45 and 55, respectively.

The binding protein can comprise (i) an extracellular domain of TCR alpha chain, TCR beta chain, TCR gamma chain, or TCR delta chain; (ii) a transmembrane domain of a TCR alpha chain, TCR beta chain, TCR gamma chain, or TCR delta chain; and/or (iii) a cytoplasmic domain of TCR alpha chain, TCR beta chain, TCR gamma chain, or TCR delta chain. The binding protein can comprise a full length or substantially full length TCR alpha chain, TCR beta chain, TCR gamma chain, and/or TCR delta chain.

In some embodiments, the binding protein comprises an amino acid sequence with one or more insertions, deletions, and/or substitutions relative to any one of SEQ ID NOs: 12, 18-22, 28-30, 38, 44-46, 48, 54-56, and 69.

For example, the binding protein can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 12, 18-22, 28-30, 38, 44-46, 48, 54-56, and 69.

In some embodiments, the binding protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 12, 18-22, 28-30, 38, 44-46, 48, 54-56, and 69.

In some embodiments, the binding protein comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 12, 18-22, 28-30, 38, 44-46, 48, 54-56, and 69.

The one or more insertions can be at the N-terminus, the C-terminus, within the amino acid sequence, or a combination thereof. The one or more insertions can be contiguous, non-contiguous, or a combination thereof.

In some embodiments, the binding protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 12, 18-22, 28-30, 38, 44-46, 48, 54-56, and 69.

In some embodiments, the binding protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 12, 18-22, 28-30, 38, 44-46, 48, 54-56, and 69.

In some embodiments, the binding protein comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 12, 18-22, 28-30, 38, 44-46, 48, 54-56, and 69.

The one or more deletions can be at the N-terminus, the C-terminus, within the amino acid sequence, or a combination thereof. The one or more deletions can be contiguous, non-contiguous, or a combination thereof.

In some embodiments, the binding protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 12, 18-22, 28-30, 38, 44-46, 48, 54-56, and 69.

In some embodiments, the binding protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 12, 18-22, 28-30, 38, 44-46, 48, 54-56, and 69.

In some embodiments, the binding protein comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 12, 18-22, 28-30, 38, 44-46, 48, 54-56, and 69.

The one or more substitutions can be at the N-terminus, the C-terminus, within the amino acid sequence, or a combination thereof. The one or more substitutions can be contiguous, non-contiguous, or a combination thereof.

In some embodiments, the binding protein comprises a TCR α chain and a TCR β chain, wherein the TCR α chain and the TCR β chain comprise or consist of amino acid sequences having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the amino acid sequences set forth in: (i) SEQ ID NOs:12 and 22, respectively; (ii) SEQ ID NOs: 20 and 30, respectively; (iii) SEQ ID NOS: 12 and 30, respectively; (iv) SEQ ID NOs:20 and 22, respectively; (v) SEQ ID NOs:38 and 48, respectively; (vi) SEQ ID NOs: 46 and 56, respectively; (vii) SEQ ID NOs:38 and 56, respectively; or (viii) SEQ ID NOs:46 and 48, respectively.

In any of the presently disclosed embodiments, a binding protein can comprise a TCR, a single-chain TCR (scTCR), a scTv, or a chimeric antigen receptor (CAR). Methods for producing engineered TCRs are described in, for example, Bowerman et al., Mol. Immunol., 46(15):3000 (2009), the techniques of which are herein incorporated by reference. Methods for making CARs are known in the art and are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al., 2007, Clin. Cancer Res. 13:5426, the techniques of which are herein incorporated by reference. In some embodiments, a binding protein comprises a soluble TCR, optionally fused to a binding domain (e.g., a scFv) specific for a CD3 protein. See Elie Dolgin, Nature Biotechnology 40:441-449 (2022).

Some examples of binding proteins are included in TABLE 2. In some embodiments, the binding protein comprises an amino acid sequence in TABLE 2. In some embodiments, the binding protein comprises an amino acid sequence that has at least 60%, at least 65%, at least 70%, at least 75%, at least 80%, at least 85%, or at least 90% sequence identity to a sequence in TABLE 2. In some embodiments, the binding protein comprises an amino acid sequence that has at least 91%, at least 92%, at least 93%, at least 94%, at least 95%, at least 96%, at least 97%, at least 98%, or at least 99% sequence identity to a sequence in TABLE 2. 99.1%, at least 99.2%, at least 99.3%, at least 99.4%, at least 99.5%, at least 99.6%, at least 99.7%, at least 99.8%, or at least 99.9% sequence identity to a sequence in TABLE 2. In some embodiments, the binding protein comprises a sequence that has at most 99.9%, at most 99.8%, at most 99.7%, at most 99.6%, at most 99.5%, at most 99.4%, at most 99.3%, at most 99.2%, or at most 99.1% to a sequence in TABLE 2. In some embodiments, the binding protein comprises a sequence that has at most 99%, at most 98%, at most 97%, at most 96%, at most 95%, at most 94%, at most 93%, at most 92%, or at most 91% to a sequence in TABLE 2. In some embodiments, the binding protein comprises a sequence that has at most 90%, at most 85%, at most 80%, at most 75%, at most 70%, at most 65%, or at most 60% sequence to a sequence in TABLE 2. In some embodiments, the binding protein comprises a sequence that has about 60%, about 65%, about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% sequence identity to a sequence in TABLE 2, or a range defined by any two of the aforementioned percentages. In some embodiments, the binding protein includes a fragment of any of the aforementioned sequences. In some embodiments, the binding protein includes any combination of any of the aforementioned sequences. Any of the aforementioned binding proteins or binding protein sequences may be useful in a method or composition described herein. For example, a binding protein may be included in a cell with a fusion protein that includes a component of CD95 (Fas) and CD137 (4-1BB) and/or a CD8αβ co-receptor (e.g. exogenous CD8αβ co-receptor).

In any of the presently disclosed embodiments, a polynucleotide encoding a binding protein can further comprise: (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii). Without being bound by theory, in certain embodiments, co-expression or concurrent expression of a binding protein and a CD8 co-receptor protein or portion thereof functional to bind to an HLA molecule may improve one or more desired activity of a host cell (e.g., immune cell, such as a T cell, optionally a CD4⁺ T cell) as compared to expression of the binding protein alone. It will be understood that the binding protein-encoding polynucleotide and the CD8 co-receptor polypeptide-encoding polynucleotide may be present on a single nucleic acid molecule (e.g., in a same expression vector), or may be present on separate nucleic acid molecules in a host cell.

In any of the presently disclosed embodiments, a CD8 co-receptor alpha chain can comprise, consist essentially of, or consist of SEQ ID NO.:87, or SEQ ID NO.:87 with the signal peptide removed. An example of a polynucleotide encoding SEQ ID NO.: 87 is provided in SEQ ID NO.:88. In some embodiments, a CD8 co-receptor alpha chain comprises, consists essentially of, or consists of an amino acid sequence with at least 70%, at least 75%, at least 80%, at least 85%, at least 90%, at least 95%, at least 97%, or at least 99% sequence identity to the amino acid sequence of SEQ ID NO.:87, or SEQ ID NO.:87 with the signal peptide removed.

In any of the presently disclosed embodiments, a CD8 co-receptor beta chain can comprise, consist essentially of, or consist of SEQ ID NO.:89, or SEQ ID NO.:89 with the signal peptide removed. An example of a polynucleotide encoding SEQ ID NO.:89 is provided in SEQ ID NO.:90. In some embodiments, a CD8 co-receptor beta chain comprises, consists essentially of, or consists of an amino acid sequence with at least 70%, at least 75%, at least 80%, at least 85%, at least 90%, at least 95%, at least 97%, or at least 99% sequence identity to the amino acid sequence of SEQ ID NO.:89, or SEQ ID NO.:89 with the signal peptide removed.

In certain further embodiments, a polynucleotide comprises: (a) the polynucleotide encoding a polypeptide comprising an extracellular portion of a CD8 co-receptor α chain; (b) the polynucleotide encoding a polypeptide comprising an extracellular portion of a CD8 co-receptor β chain; and (c) a polynucleotide encoding a self-cleaving peptide disposed between the polynucleotide of (a) and the polynucleotide of (b). In further embodiments, a polynucleotide comprises a polynucleotide that encodes a self-cleaving peptide and is disposed between: (1) the polynucleotide encoding a binding protein and the polynucleotide encoding a polypeptide comprising an extracellular portion of a CD8 co-receptor α chain; and/or (2) the polynucleotide encoding a binding protein and the polynucleotide encoding a polypeptide comprising an extracellular portion of a CD8 co-receptor β chain.

In still further embodiments, a polynucleotide can comprise, operably linked in-frame: (i) (pnCD8α)-(pnSCP1)-(pnCD8β)-(pnSCP2)-(pnBP); (ii) (pnCD8β)-(pnSCP1)-(pnCD8α)-(pnSCP2)-(pnBP); (iii) (pnBP)-(pnSCP1)-(pnCD8α)-(pnSCP2)-(pnCD8β); (iv) (pnBP)-(pnSCP1)-(pnCD8β)-(pnSCP2)-(pnCD8α); (v) (pnCD8α)-(pnSCP1)-(pnBP)-(pnSCP2)-(pnCD8β); or (vi) (pnCD8β)-(pnSCP1)-(pnBP)-(pnSCP2)-(pnCD8a), wherein pnCD8α is the polynucleotide encoding a polypeptide that comprises an extracellular portion of a CD8 co-receptor α chain, wherein pnCD8β is the polynucleotide encoding a polypeptide that comprises an extracellular portion of a CD8 co-receptor α chain, wherein pnBP is the polynucleotide encoding a binding protein, and wherein pnSCP1 and pnSCP2 are each independently a polynucleotide encoding a self-cleaving peptide, wherein the polynucleotides and/or the encoded self-cleaving peptides are optionally the same or different (e.g., P2A, T2A, F2A, E2A).

It will be understood that self-cleaving peptide can comprise a linker N-terminal and/or C-terminal thereto. An example of a linker is GSG. In some embodiments, a T2A peptide is provided that comprises a N-terminal GSG linker. In some embodiments, the GSG-T2A sequence comprises, consists essentially of, or consists of GSG and the amino acid sequence of SEQ ID NO.:75. In some embodiments, a GSG-P2A sequence comprises, consists essentially of, or consists of SEQ ID NO.:74.

In certain embodiments, the encoded binding protein comprises a TCRα chain and a TCRβ chain, wherein the polynucleotide comprises a polynucleotide encoding a self-cleaving peptide disposed between the polynucleotide encoding a TCRα chain and the polynucleotide encoding a TCRβ chain. In further embodiments, the polynucleotide comprises, operably linked in-frame: (i) (pnCD8α)-(pnSCP1)-(pnCD8β)-(pnSCP2)-(pnTCRβ)-(pnSCP3)-(pnTCRα); (ii)(pnCD8β)-(pnSCP1)-(pnCD8α)-(pnSCP2)-(pnTCRβ)-(pnSCP3)-(pnTCRα); (iii) (pnCD8α)-(pnSCP1)-(pnCD8β)-(pnSCP2)-(pnTCRα)-(pnSCP3)-(pnTCRβ); (iv) (pnCD8β)-(pnSCP1)-(pnCD8α)-(pnSCP2)-(pnTCRα)-(pnSCP3)-(pnTCRβ); (v) (pnTCRβ)-(pnSCP1)-(pnTCRα)-(pnSCP2)-(pnCD8α)-(pnSCP3)-(pnCD8β); (vi) (pnTCRβ)-(pnSCP1)-(pnTCRα)-(pnSCP2)-(pnCD8β)-(pnSCP3)-(pnCD8α); (vii) (pnTCRα)-(pnSCP1)-(pnTCRβ)-(pnSCP2)-(pnCD8α)-(pnSCP3)-(pnCD8β); (viii) (pnTCRα)-(pnSCP1)-(pnTCRβ)-(pnSCP2)-(pnCD8β)-(pnSCP3)-(pnCD8α), wherein pnCD8α is the polynucleotide encoding a polypeptide that comprises an extracellular portion of a CD8 co-receptor α chain,
wherein pnCD8β is the polynucleotide encoding a polypeptide that comprises an extracellular portion of a CD8 co-receptor α chain, wherein pnTCRα is the polynucleotide encoding a TCR α chain, wherein pnTCRβ is the polynucleotide encoding a TCR β chain, and wherein pnSCP1, pnSCP2, and pnSCP3 are each independently a polynucleotide encoding a self-cleaving peptide, wherein the polynucleotides and/or the encoded self-cleaving peptides are optionally the same or different.

In certain embodiments, an encoded polypeptide of the present disclosure comprises one or more junction amino acids. “Junction amino acids” or “junction amino acid residues” refer to one or more (e.g., 2 to about 10) amino acid residues between two adjacent motifs, regions or domains of a polypeptide, such as between a binding domain and an adjacent constant domain or between a TCR chain and an adjacent self-cleaving peptide. Junction amino acids can result from the design of a construct that encodes a fusion protein (e.g., amino acid residues resulting from the use of a restriction enzyme site during the construction of a nucleic acid molecule encoding a fusion protein), or from cleavage of, for example, a self-cleaving peptide adjacent one or more domains of an encoded binding protein of this disclosure (e.g., a P2A peptide disposed between a TCR α-chain and a TCR β-chain, the self-cleavage of which can leave one or more junction amino acids in the α-chain, the TCR β-chain, or both).

In further embodiments, a binding protein is expressed as part of a transgene construct that encodes, and/or a host cell of the present disclosure can encode: one or more additional accessory protein, such as a safety switch protein; a tag, a selection marker; a CD8 co-receptor β-chain; a CD8 co-receptor α-chain or both; or any combination thereof. Polynucleotides and transgene constructs useful for encoding and expressing binding proteins and accessory components (e.g., one or more of a safety switch protein, a selection marker, CD8 co-receptor β-chain, or a CD8 co-receptor α-chain) are described in PCT application PCT/US2017/053112, the polynucleotides, transgene constructs, and accessory components, including the nucleotide and amino acid sequences, of which are hereby incorporated by reference. It will be understood that any or all of a binding protein of the present disclosure, a safety switch protein, a tag, a selection marker, a CD8 co-receptor β-chain, or a CD8 co-receptor α-chain may be encoded by a single nucleic acid molecule or may be encoded by polynucleotide sequences that are, or are present on, separate nucleic acid molecules.

Example safety switch proteins include, for example, a truncated EGF receptor polypeptide (huEGFRt) that is devoid of extracellular N-terminal ligand binding domains and intracellular receptor tyrosine kinase activity, but that retains its native amino acid sequence, has type I transmembrane cell surface localization, and has a conformationally intact binding epitope for pharmaceutical-grade anti-EGFR monoclonal antibody, cetuximab (Erbitux) tEGF receptor (tEGFr; Wang et al., Blood 118:1255-1263, 2011); a caspase polypeptide (e.g., iCasp9; Straathof et al., Blood 105:4247-4254, 2005; Di Stasi et al., N. Engl. J Med. 365:1673-1683, 2011; Zhou and Brenner, Exp. Hematol. pii:S0301-472X(16)30513-6. doi:10.1016/j.exphem.2016.07.011), RQR8 (Philip et al., Blood 124:1277-1287, 2014); a 10-amino-acid tag derived from the human c-myc protein (Myc) (Kieback et al., Proc. Natl. Acad. Sci. USA 105:623-628, 2008); and a marker/safety switch polypeptide, such as RQR (CD20+CD34; Philip et al., 2014).

Other accessory components useful for modified host cells of the present disclosure comprise a tag or selection marker that allows the cells to be identified, sorted, isolated, enriched, or tracked. For example, marked host cells having desired characteristics (e.g., an antigen-specific TCR and a safety switch protein) can be sorted away from unmarked cells in a sample and more efficiently activated and expanded for inclusion in a product of desired purity.

As used herein, the term “selection marker” comprises a nucleic acid construct (and the encoded gene product) that confers an identifiable change to a cell permitting detection and positive selection of immune cells transduced with a polynucleotide comprising a selection marker. RQR is a selection marker that comprises a major extracellular loop of CD20 and two minimal CD34 binding sites. In some embodiments, an RQR-encoding polynucleotide comprises a polynucleotide that encodes the 16-amino-acid CD34 minimal epitope. In some embodiments, the CD34 minimal epitope is incorporated at the amino terminal position of a CD8 co-receptor stalk domain (Q8). In further embodiments, the CD34 minimal binding site sequence can be combined with a target epitope for CD20 to form a compact marker/suicide gene for T cells (RQR8) (Philip et al., 2014, incorporated by reference herein). This construct allows for the selection of host cells expressing the construct, with for example, CD34 specific antibody bound to magnetic beads (Miltenyi) and that utilizes clinically accepted pharmaceutical antibody, rituximab, that allows for the selective deletion of a transgene expressing engineered T cell (Philip et al., 2014).

Further example selection markers also include several truncated type I transmembrane proteins normally not expressed on T cells: the truncated low-affinity nerve growth factor, truncated CD19, and truncated CD34 (see for example, Di Stasi et al., N. Engl. J. Med. 365:1673-1683, 2011; Mavilio et al., Blood 83:1988-1997, 1994; Fehse et al., Mol. Ther. 1:448-456, 2000; each incorporated herein in their entirety). A useful feature of CD19 and CD34 is the availability of the off-the-shelf Miltenyi CliniMACs™ selection system that can target these markers for clinical-grade sorting. However, CD19 and CD34 are relatively large surface proteins that may tax the vector packaging capacity and transcriptional efficiency of an integrating vector. Surface markers containing the extracellular, non-signaling domains or various proteins (e.g., CD19, CD34, LNGFR) also can be employed. Any selection marker may be employed (e.g., one acceptable for Good Manufacturing Practices). In certain embodiments, selection markers are expressed with a polynucleotide that encodes a gene product of interest (e.g., a binding protein of the present disclosure, such as a TCR or CAR). Further examples of selection markers include, for example, reporters such as GFP, EGFP, β-gal or chloramphenicol acetyltransferase (CAT). In certain embodiments, a selection marker, such as, for example, CD34 is expressed by a cell and the CD34 can be used to select enrich for, or isolate (e.g., by immunomagnetic selection) the transduced cells of interest for use in the methods described herein. As used herein, a CD34 marker is distinguished from an anti-CD34 antibody, or, for example, a scFv, TCR, or other antigen recognition moiety that binds to CD34.

In certain embodiments, a selection marker comprises an RQR polypeptide, a truncated low-affinity nerve growth factor (tNGFR), a truncated CD19 (tCD19), a truncated CD34 (tCD34), or any combination thereof.

Regarding RQR polypeptides, without wishing to be bound by theory, it is believed that distance from the host cell surface is important for RQR polypeptides to function as selection markers/safety switches (Philip et al., 2010 (supra)). In some embodiments, the encoded RQR polypeptide is contained in a β-chain, an α-chain, or both, or a fragment or variant of either or both, of the encoded CD8 co-receptor. In specific embodiments, a modified host cell comprises a heterologous polynucleotide encoding iCasp9 and a heterologous polynucleotide encoding a recombinant CD8 co-receptor protein that comprises a β-chain containing a RQR polypeptide and further comprises a CD8 α-chain.

An encoded CD8 co-receptor includes, in some embodiments, an α-chain or a fragment or variant thereof. An amino acid sequence of the human CD8 co-receptor α-chain precursor is known and is provided at, for example, UniProtKB-P30433 (see also UniProtKB-P31783; -P10732; and -P10731). An encoded CD8 co-receptor includes, in some embodiments, a 1-chain or a fragment or variant thereof. An amino acid sequence of the human CD8 co-receptor β-chain precursor is known and is provided at, for example, UniProtKB-P10966 (see also UniProtKB-Q9UQ56; -E9PD41; Q8TD28; and -P30434; and -P05541).

An isolated polynucleotide of this disclosure may further comprise a polynucleotide encoding a safety switch protein, a selection marker, a CD8 co-receptor beta chain, or a CD8 co-receptor alpha chain as disclosed herein, or may comprise a polynucleotide encoding any combination thereof.

In any of the presently disclosed embodiments, a polynucleotide can be codon optimized for expression in a host cell. In some embodiments, the host cell comprises a human immune system cell, such as a T cell, a NK cell, or a NK-T cell (Scholten et al., Clin. Immunol. 119:135, 2006). Codon optimization can be performed using known techniques and tools, e.g., using the GenScript® OptimumGene™ tool, or GeneArt (Life Technologies). Codon-optimized sequences include sequences that are partially codon-optimized (i.e., one or more of the codons is optimized for expression in the host cell) and those that are fully codon-optimized. It will be appreciated that in embodiments wherein a polynucleotide encodes more than one polypeptide (e.g., a TCR α chain, a TCR β chain, a CD8 co-receptor α chain, a CD8 co-receptor β chain, and one or more self-cleaving peptides), each polypeptide can independently fully codon optimized, partially codon optimized, or not codon optimized.

Amino acid and polynucleotide sequences for example binding proteins “11N4A” and “11N6” are shown in Table 1.

TABLE 1
Certain Polynucleotide and Amino Acid Sequences
related to TCRs 11N4A and 11N6

| TCR | |
|---|---|
| 11N4A | 11N6 |

Polynucleotide Sequences

| | | |
|---|---|---|
| TCR α-chain with signal peptide, original polynucleotide | 5 | 33 |
| TCR β-chain with signal peptide, original polynucleotide | 6 | 34 |
| TCRβ-P2A-TCRα, codon-optimization (A) | 7 | 35 |
| TCRβ-P2A-TCRα, codon-optimization (B) | 8 | — |
| CD8α-T2A-CD8β-P2A-TCRβ-P2A-TCRα, codon- | 9 | 36 |
| optimization (A) | | |
| CD8α-T2A-CD8β-P2A-TCRβ-P2A-TCRα, codon- | 10 | — |
| optimization (B) | | |

Amino acid Sequences

| | | |
|---|---|---|
| TCR α-chain with signal peptide, original | 11 | 37 |
| TCR α-chain without signal peptide, original | 12 | 38 |
| TCR α-chain variable domain, without signal peptide | 13 | 39 |
| TCR α-chain variable domain, CDR1α | 14 | 40 |
| TCR α-chain variable domain, CDR2α | 15 | 41 |
| TCR α-chain variable domain, CDR3α - IMGT junction | 16 | 42 |
| TCR α-chain variable domain, CDR3α - IMGT | 17 | 43 |
| TCR α-chain constant domain, original | 18 | 44 |
| TCR α-chain constant domain, cys-modified | 19 | 45 |
| TCR α-chain without signal peptide, cys-modified | 20 | 46 |
| TCR β-chain with signal peptide, original | 21 | 47 |
| TCR β-chain without signal peptide, original | 22 | 48 |
| TCR β-chain variable domain, without signal peptide | 23 | 49 |
| TCR β-chain variable domain, CDR1β | 24 | 50 |
| TCR β-chain variable domain, CDR2β | 25 | 51 |
| TCR β-chain variable domain, CDR3β - IMGT junction | 26 | 52 |
| TCR β-chain variable domain, CDR3β - IMGT | 27 | 53 |
| TCR β-chain constant domain, original | 28 | 54 |
| TCR β-chain constant domain, cys-modified | 29 | 55 |
| TCR β-chain without signal peptide, cys-modified | 30 | 56 |
| TCRβ-P2A-TCRα | 31 | 57 |
| CD8α-T2A-CD8β-P2A-TCRβ-P2A-TCRα | 32 | 58 |

### Vectors

In another aspect, the present disclosure provides an expression vector, comprising any polynucleotide as provided herein operably linked to an expression control sequence.

Also provided herein are vectors that comprise a polynucleotide or transgene construct of the instant disclosure. Some examples of vectors include plasmids, viral vectors, cosmids, and others. Some vectors may be capable of autonomous replication in a host cell into which they are introduced (e.g., bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding polypeptides as described herein) are co administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a vector selected from lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing TCR or CAR transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as Sleeping Beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

In certain embodiments, a vector is capable of delivering the polynucleotide or transgene construct to a host cell (e.g., a hematopoietic progenitor cell or a human immune system cell). In specific embodiments, a vector is capable of delivering a polynucleotide or transgene construct to human immune system cell, such as, for example, a CD4⁺ T cell, a CD8⁺ T cell, a CD4⁻ CD8⁻ double negative T cell, a stem cell memory T cell, a γδ T cell, a natural killer cell, a dendritic cell, or any combination thereof. In further embodiments, a vector is capable of delivering a transgene construct to a naïve T cell, a central memory T cell, an effector memory T cell, or any combination thereof. In some embodiments, a vector that encodes a polynucleotide or transgene construct of the present disclosure may further comprise a polynucleotide that encodes a nuclease that can be used to perform a chromosomal knockout in a host cell (e.g., a CRISPR-Cas endonuclease or another endonuclease as disclosed herein) or that can be used to deliver a therapeutic polynucleotide or transgene or portion thereof to a host cell in a gene therapy replacement or gene repair therapy. Alternatively, a nuclease used for a chromosomal knockout or a gene replacement or gene repair therapy can be delivered to a host cell independent of a vector that encodes a polynucleotide or transgene construct of this disclosure.

In certain embodiments, the vector is capable of delivering the polynucleotide to a host cell. In further embodiments, the host cell is a hematopoietic progenitor cell or a human immune system cell. In still further embodiments, the human immune system cell is a CD4+ T cell, a CD8+ T cell, a CD4−CD8− double negative T cell, a γδ T cell, a natural killer cell, a natural killer T cell, a macrophage, a monocyte, a dendritic cell, or any combination thereof. In yet further embodiments, the T cell is a naïve T cell, a central memory T cell, an effector memory T cell, or any combination thereof.

In any of the presently disclosed embodiments, the vector is a viral vector. In certain embodiments, the viral vector is a lentiviral vector or a γ-retroviral vector.

### Host Cells

Also provided herein are host cells that encode and/or express a binding protein (and, optionally, one or more accessory protein, such as a transduction marker, a CD8 co-receptor polypeptide, or the like, as provided herein). In certain embodiments, a host cell is provided that is modified to comprise a polynucleotide and/or an expression vector of the present disclosure, and/or to express a binding protein of the present disclosure.

Any suitable host cell may be modified to include a heterologous polynucleotide encoding a binding protein of this disclosure, including, for example, an immune cell, such as T cell, a NK cell, or a NK-T cell. In some embodiments, a modified immune cell comprises a CD4⁺ T cell, a CD8⁺ T cell, or both. Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein.

Any appropriate method can be used to transfect or transduce the cells, for example, the T cells, or to administer the polynucleotides or compositions of the present methods. Known methods for delivering polynucleotides to host cells include, for example, use of cationic polymers, lipid-like molecules, and certain commercial products such as, for example, IN-VIVO-JET PEI. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein.

In certain embodiments, the host cell or modified cell can be a peripheral blood mononuclear cell (PBMC). A host cell can be a lymphoid cell. A host cell can be a lymphocyte. In some embodiments, the host cell or modified cell can be a hematopoietic progenitor cell and/or or human immune cell. In some embodiments, the immune cell comprises a T cell, a NK cell, a NK-T cell, a dendritic cell, a macrophage, a monocyte, or any combination thereof. In some embodiments, the host or modified cell is a mammalian cell (e.g., a human cell or mouse cell). In further embodiments, the immune cell comprises a CD4+ T cell, a CD8+ T cell, a CD4− CD8− double negative T cell, a γδ T cell, or any combination thereof. In certain further embodiments, the immune cell comprises a CD4+ T cell and a CD8+ T cell. In certain still further embodiments, the CD4+ T cell, the CD8+ T cell, or both comprise (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii).

In any of the foregoing embodiments, a host cell (e.g., an immune cell) may be modified to reduce or eliminate expression of one or more endogenous genes that encode a polypeptide involved in immune signaling or other related activities. Example gene knockouts include those that encode PD-1, LAG-3, CTLA4, TIM3, TIGIT, FasL, an HLA molecule, a TCR molecule, or the like. Without wishing to be bound by theory, certain endogenously expressed immune cell proteins may be recognized as foreign by an allogeneic host receiving the modified immune cells, which may result in elimination of the modified immune cells (e.g., an HLA allele), or may downregulate the immune activity of the modified immune cells (e.g., PD-1, LAG-3, CTLA4, FasL, TIGIT, TIM3), or may interfere with the binding activity of a heterologously expressed binding protein of the present disclosure (e.g., an endogenous TCR of a modified T cell that binds a, e.g., non-Ras antigen and thereby interferes with the modified immune cell binding a cell that expresses a e.g., Ras antigen).

Accordingly, decreasing or eliminating expression or activity of such endogenous genes or proteins can improve the activity, tolerance, or persistence of the modified cells in an autologous or allogeneic host setting and may allow for universal administration of the cells (e.g., to any recipient regardless of HLA type). In certain embodiments, a modified cell is a donor cell (e.g., allogeneic) or an autologous cell. In certain embodiments, a modified cell of this disclosure comprises a chromosomal gene knockout of one or more of a gene that encodes PD-1, LAG-3, CTLA4, TIM3, TIGIT, FasL, an HLA component (e.g., a gene that encodes an al macroglobulin, an α2 macroglobulin, an α3 macroglobulin, a β1 microglobulin, or a 32 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24):5697 (2012); and Torikai et al., Blood 122(8):1341 (2013), the gene-editing techniques, compositions, and adoptive cell therapies of which are herein incorporated by reference in their entirety).

As used herein, the term “chromosomal gene knockout” generally refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in,” for target gene “knock-out,” and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) generally refers to a fusion protein comprising a zinc finger DNA-binding domain fused to a non-specific DNA cleavage domain, such as a FokI endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) generally refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histidine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little, or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system generally refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Example gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

As used herein, a “meganuclease,” also referred to as a “homing endonuclease,” generally refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Example meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from PD-1, LAG3, TIM3, CTLA4, TIGIT, FasL, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (e.g., of PD-1, TIM3, LAG3, CTLA4, TIGIT, FasL, an HLA component, or a TCR component, or any combination thereof) in the host cell.

In certain embodiments, a gene knockout comprises an insertion, a deletion, a mutation or a combination thereof, and made using a CRISPR/Cas nuclease system or base editing system (Komor, A. C.; Kim, Y. B.; Packer, M. S.; Zuris, J. A.; Liu, D. R. Nature 533, 420-424 (2016). Briefly, base editing is a genome-editing approach that uses components from CRISPR systems together with other enzymes to directly introduce point mutations into cellular DNA or RNA without making double-stranded DNA breaks. Certain DNA base editors comprise a catalytically disabled nuclease fused to a nucleobase deaminase enzyme and, in some cases, a DNA glycosylase inhibitor. RNA base editors function similarly, using components that target RNA. Base editors directly convert one base or base pair into another, enabling the efficient installation of point mutations in non-dividing cells without generating excess undesired editing by-products. See e.g., Rees H et al. Nature Reviews Genetics (2018).

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In certain embodiments, a chromosomal gene knockout comprises a knockout of an HLA component gene selected from an al macroglobulin gene, an α2 macroglobulin gene, an α3 macroglobulin gene, a β1 microglobulin gene, or a β2 microglobulin gene.

In certain embodiments, a chromosomal gene knockout comprises a knockout of a TCR component gene selected from a TCR α variable region gene, a TCR β variable region gene, a TCR constant region gene, or a combination thereof.

In some embodiments, a population of host cells comprising a binding protein disclosed herein exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, or at least 5000 fold increased functional avidity for a target antigen of the binding protein as compared to a population of control cells (for example, cells expressing a control binding protein specific for the same target antigen). The host cells can comprise a binding protein (e.g., a TCR comprising Vα and Vβ regions and/or CDRs disclosed herein) that binds a target antigen (for example, a neoantigen (e.g., p53 PIK3CA, NRAS, HRAS, or KRAS (e.g., a KRAS G12 mutant peptide, such as KRAS G12V mutant peptide, e.g., present in a peptide:HLA complex)). The increase in avidity can be, for example, as determined by an assay for determining expression an activation marker (e.g., CD137, CD69, Granzyme B, CD107a, IFN-gamma, TNF-α, IL-12, a cytokine, an interleukin, an interferon) upon exposure to target cells that express or present the target antigen, or and/or an assay to determine EC50 (e.g., peptide dose at which a half-maximal activation of a T cell population is reached).

### Host Cell Compositions and Unit Doses

In another aspect, compositions and unit doses are provided herein that comprise a modified host cell of the present disclosure and a pharmaceutically acceptable carrier, diluent, or excipient.

In certain embodiments, a host cell composition or unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of peripheral blood mononuclear cells (PBMCs).

In some embodiments, a host cell composition or unit dose comprises (i) a composition comprising at least about 50% modified CD4⁺ T cells, combined with (ii) a composition comprising at least about 50% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10ratio, wherein the host cell composition or unit dose contains a reduced amount or substantially no naïve T cells. In further embodiments, a host cell composition or unit dose comprises (i) a composition comprising at least about 60% modified CD4⁺ T cells, combined with (ii) a composition comprising at least about 60% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10ratio, wherein the unit dose contains a reduced amount or substantially no naïve T cells. In still further embodiments, a host cell composition or unit dose comprises (i) a composition comprising at least about 70% engineered CD4⁺ T cells, combined with (ii) a composition comprising at least about 70% engineered CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10ratio, wherein the unit dose contains a reduced amount or substantially no naïve T cells. In some embodiments, a host cell composition or unit dose comprises (i) a composition comprising at least about 80% modified CD4⁺ T cells, combined with (ii) a composition comprising at least about 80% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the host cell composition or unit dose contains a reduced amount or substantially no naïve T cells. In some embodiments, a host cell composition or unit dose comprises (i) a composition comprising at least about 85% modified CD4⁺ T cells, combined with (ii) a composition comprising at least about 85% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the host cell composition or unit dose contains a reduced amount or substantially no naïve T cells. In some embodiments, a host cell composition or unit dose comprises (i) a composition comprising at least about 90% modified CD4⁺ T cells, combined with (ii) a composition comprising at least about 90% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the host cell composition or unit dose contains a reduced amount or substantially no naïve T cells.

In some embodiments, the composition comprises a CD4+ cell population comprising (i) at least about 30%, at least about 40%, at least about 50%, at least about 60%, at least about 70%, at least about 80%, at least about 85%, at least about 90%, or at least about 95% modified CD4+ T cells. In some embodiments, the composition further comprises a CD8+ cell population comprising (ii) at least about 30%, at least about 40%, at least about 50%, at least about 60%, at least about 70%, at least about 80%, at least about 85%, at least about 90%, or at least about 95% modified CD8⁺ T cells.

In some embodiments, a host cell composition or unit dose comprises about a 1:1 ratio, about a 1:2 ratio, about a 1:3 ratio, about a 1:4 ratio, about a 1:5 ratio, about a 1:6 ratio, about a 1:7 ratio, about a 1:8 ratio, about a 1:9 ratio, about a 1:10 ratio, about a 2:1 ratio, about a 3:1 ratio, about a 4:1 ratio, about a 5:1 ratio, about a 6:1 ratio, about a 7:1 ratio, about an 8:1 ratio, about a 9:1 ratio, about a 10:1 ratio, about a 3:2 ratio, or about a 2:3 ratio of CD4+ to CD8+ T cells (for example, of CD4+ T cells modified to comprise or express a binding protein disclosed herein to CD8+ T cells modified to comprise or express a binding protein disclosed herein).

In some embodiments, a host cell composition or unit dose comprises ratio of CD4+ to CD8+ T cells that is at least 1:1, at least 1:2, at least 1:3, at least 1:4, at least 1:5, at least 1:6, at least 1:7, at least 1:8, at least 1:9, at least 1:10, at least 2:1, at least 3:1, at least 4:1, at least 5:1, at least 6:1, at least 7:1, at least 8:1, at least 9:1, at least 10:1, at least 3:2, or at least 2:3.

In some embodiments, a host cell composition or unit dose comprises ratio of CD4+ to CD8+ T cells that is at most 1:1, at most 1:2, at most 1:3, at most 1:4, at most 1:5, at most 1:6, at most 1:7, at most 1:8, at most 1:9, at most 1:10, at most 2:1, at most 3:1, at most 4:1, at most 5:1, at most 6:1, at most 7:1, at most 8:1, at most 9:1, at most 10:1, at most 3:2, or at most 2:3.

In some embodiments, a host cell composition or unit dose comprises ratio of CD4+ to CD8+ T cells that is between about 1:10 and 10:1, 1:10 and 8:1, 1:10 and 7:1, 1:10 and 6:1, 1:10 and 5:1, 1:10 and 4:1, 1:10 and 3:1, 1:10 and 2:1, 1:10 and 1:1, 1:10 and 1:2, 1:10 and 1:3, 1:10 and 1:4, 1:10 and 1:5, 1:10 and 1:7, 1:5 and 10:1, 1:5 and 8:1, 1:5 and 7:1, 1:5 and 6:1, 1:5 and 5:1, 1:5 and 4:1, 1:5 and 3:1, 1:5 and 2:1, 1:5 and 1:1, 1:5 and 1:2, 1:5 and 1:3, 1:5 and 1:4, 1:3 and 10:1, 1:3 and 8:1, 1:3 and 7:1, 1:3 and 6:1, 1:3 and 5:1, 1:3 and 4:1, 1:3 and 3:1, 1:3 and 2:1, 1:3 and 1:1, 1:3 and 1:2, 1:2 and 10:1, 1:2 and 8:1, 1:2 and 7:1, 1:2 and 6:1, 1:2 and 5:1, 1:2 and 4:1, 1:2 and 3:1, 1:2 and 2:1, 1:2 and 1:1, 1:1 and 10:1, 1:1 and 8:1, 1:1 and 7:1, 1:1 and 6:1, 1:1 and 5:1, 1:1 and 4:1, 1:1 and 3:1, 1:1 and 2:1, 2:1 and 10:1, 2:1 and 8:1, 2:1 and 7:1, 2:1 and 6:1, 2:1 and 5:1, 2:1 and 4:1, 2:1 and 3:1, 3:1 and 10:1, 3:1 and 8:1, 3:1 and 7:1, 3:1 and 6:1, 3:1 and 5:1, 3:1 and 4:1, 5:1 and 10:1, 5:1 and 8:1, 5:1 and 7:1, or 5:1 and 6:1.

CD4+ T cells in a composition, host cell composition, or unit dose can be CD4+ T cells that are modified or engineered to express a CD8 co-receptor disclosed herein, for example, using a vector or polynucleotide disclosed herein.

It will be appreciated that a host cell composition or unit dose of the present disclosure may comprise any host cell as described herein, or any combination of host cells. In certain embodiments, for example, a host cell composition or unit dose comprises modified CD8+ T cells, modified CD4+ T cells, or both, wherein these T cells are modified to encode a binding protein specific for a Ras peptide:HLA-A*11:01 complex. In addition or alternatively, a host cell composition or unit dose of the present disclosure can comprise any host cell or combination of host cells as described herein, and can further comprise a modified cell (e.g., immune cell, such as a T cell) expressing a binding protein specific for a different antigen (e.g., a different Ras antigen, or an antigen from a different protein or target, such as, for example, BCMA, CD3, CEACAM6, c-Met, EGFR, EGFRvIII, ErbB2, ErbB3, ErbB4, EphA2, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, TNFR1, TNFR2, PD1, PD-L1, PD-L2, HVEM, MAGE-A (e.g., including MAGE-A1, MAGE-A3, and MAGE-A4), mesothelin, NY-ESO-1, PSMA, RANK, ROR1, TNFRSF4, CD40, CD137, TWEAK-R, HLA, tumor- or pathogen-associated peptide bound to HLA, hTERT peptide bound to HLA, tyrosinase peptide bound to HLA, WT-1 peptide bound to HLA, LTβR, LIFRβ, LRP5, MUC1, OSMRβ, TCRα, TCRβ, CD19, CD20, CD22, CD25, CD28, CD30, CD33, CD52, CD56, CD79a, CD79b, CD80, CD81, CD86, CD123, CD171, CD276, B7H4, TLR7, TLR9, PTCH1, WT-1, HA¹-H, Robo1, α-fetoprotein (AFP), Frizzled, OX40, PRAME, and SSX-2. or the like). In some embodiments, the binding protein binds to a peptide (e.g., the different antigens presented above) complexed with an HLA protein, e.g., an HLA-A, -B, -C, E, -G, -H, -J, -K, or -L. For example, a unit dose can comprise modified CD8⁺ T cells expressing a binding protein that specifically binds to a Ras-HLA complex and modified CD4⁺ T cells (and/or modified CD8⁺ T cells) expressing a binding protein (e.g., a CAR) that specifically binds to a PSMA antigen. It will also be appreciated that any of the host cells disclosed herein may be administered in a combination therapy.

In any of the embodiments described herein, a host cell composition or unit dose comprises equal, or approximately equal numbers of engineered CD45RA⁻ CD3⁺ CD8⁺ and modified CD45RA⁻ CD3⁺ CD4⁺ T_{M} cells.

In any of the embodiments described herein, a host cell composition or unit dose comprises one or more populations of cells (e.g., CD4+ or CD8+ cells) that have undergone CD62L positive selection, for example, to improve in vivo persistence.

Host cells can be genetically engineered to comprise or express a binding protein ex vivo, in vitro, or in vivo.

### Uses

In additional aspects, the present disclosure provides methods for treating or for preventing a relapse of a disease or disorder associated with a KRAS G12V or a NRAS G12V mutation or a HRAS G12V mutation in a subject. Such diseases or disorders include, for example, cancers, such as solid cancers and hematological malignancies. In certain example embodiments, the disease or disorder comprises a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Myelomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatosis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma.

“Treat” or “treatment” or “ameliorate” generally refers to medical management of a disease, disorder, or condition of a subject (e.g., a human or non-human mammal, such as a primate, horse, cat, dog, goat, mouse, or rat). In general, an appropriate dose or treatment regimen comprising a composition (e.g., comprising a binding protein, polynucleotide, vector, host cell, host cell composition, unit dose, and/or immunogenic polypeptide) of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

A “therapeutically effective amount” or “effective amount”, as used herein, generally refers to an amount of a composition sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient.

The term “pharmaceutically acceptable excipient or carrier” or “physiologically acceptable excipient or carrier” generally refer to biologically compatible vehicles, e.g., physiological saline, which are described in greater detail herein, that are suitable for administration to a human or other non-human mammalian subject and generally recognized as safe or not causing a serious adverse event.

As used herein, “statistically significant” generally refers to a p value of 0.050 or less when calculated using the Students t-test or to values or indicators of statistical significance using another appropriate statistical test and indicates that it is unlikely that a particular event or result being measured has arisen by chance.

Subjects that can be treated according to the current disclosure are, in general, human and other primate subjects, such as monkeys and apes for veterinary medicine purposes. In any of the aforementioned embodiments, the subject may be a human subject. The subject can be a mammal. The subjects can be male or female and can be any suitable age, including infant, juvenile, adolescent, adult, and geriatric subjects. Compositions according to the present disclosure may be administered in a manner appropriate to the disease, condition, or disorder to be treated as determined by persons skilled in the medical art. In any of the above embodiments, a modified host cell, host cell composition, or unit dose as described herein is administered intravenously, intraperitoneally, intratumorally, into the bone marrow, into a lymph node, or into the cerebrospinal fluid so as to encounter target cells (e.g., leukemia cells). An appropriate dose, suitable duration, and frequency of administration of the compositions will be determined by such factors as a condition of the patient; size, type, and severity of the disease, condition, or disorder; the particular form of the active ingredient; and the method of administration.

As used herein, the term “adoptive immune therapy” or “adoptive immunotherapy” generally refers to administration of naturally occurring or genetically engineered, disease- or antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

In some embodiments, the subject expresses a Ras antigen comprising or consisting of the amino acid sequence set forth in any one of SEQ ID NOs:2-3.

In some embodiments, the subject is HLA-A⁺, HLA-B⁺, or HLA-C⁺. In some embodiments, the subject is HLA-A*11:01⁺

In certain embodiments, a method comprises determining the HLA type or types of a subject and/or identifying the presence of a neoantigen, prior to administering therapy according to the present disclosure.

Expression of an HLA allele can be determined by, for example, genetic sequencing (e.g., high throughput Next Generation Sequencing (NGS)). This genetic determination of the HLA expression is referred to herein as “HLA typing” and can determined though molecular approaches in a clinical laboratory licensed for HLA typing. In some embodiments, HLA typing is performed using PCR amplification followed by high throughput NGS and subsequent HLA determination. Herein, the HLA haplotype can be determined at the major HLA loci (e.g., HLA-A, HLA-B, HLA-C, etc.).

HLA typing can be performed using any known method, including, for example, protein or nucleic acid testing. Examples of nucleic acid testing include sequence-based typing (SBT) and use of sequence-specific oligonucleotide probes (SSOP) or sequence-specific primers (SSP). In certain embodiments, HLA typing is performed using PCR amplification followed by high throughput Next Generation Sequencing (NGS) and subsequent HLA determination. In some embodiments, sequence typing is performed using a system available through Scisco Genetics (sciscogenetics.com/pages/technology.html, the contents of which is incorporated herein by reference in its entirety). Other methods for HLA typing include, e.g., those disclosed in Mayor et al. PLoS One 10(5):e0127153 (2015), which methods and reagents are incorporated herein by reference.

In particular embodiments, a method comprises administering a composition comprising modified CD8+ and/or modified CD4+ T cells that comprise a heterologous polynucleotide encoding a second binding protein as provided herein.

In the case of host cell compositions or unit doses, the amount of cells therein is at least one cell (for example, one modified CD8⁺ T cell subpopulation (e.g., optionally comprising memory and/or naïve CD8⁺ T cells); one modified CD4⁺ T cell subpopulation (e.g., optionally comprising memory and/or naïve CD4⁺ T cells)) or is more typically greater than 10² cells, for example, up to 10⁴, up to 10⁵, up to 10⁶, up to 10⁷, up to 10⁸, up to 10⁹, or more than 10¹⁰ cells. In certain embodiments, the cells are administered in a range from about 10⁴ to about 10¹⁰ cells/m², or in a range of about 10⁵ to about 10⁹ cells/m². In some embodiments, an administered dose comprises up to about 3.3×10⁵ cells/kg. In some embodiments, an administered dose comprises up to about 1×10⁶ cells/kg. In some embodiments, an administered dose comprises up to about 3.3×10⁶ cells/kg. In some embodiments, an administered dose comprises up to about 1×10⁷ cells/kg. In certain embodiments, a modified immune cell is administered to a subject at a dose comprising up to about 5×10⁴ cells/kg, 5×10⁵ cells/kg, 5×10⁶ cells/kg, or up to about 5×10⁷ cells/kg. In certain embodiments, a modified immune cell is administered to a subject at a dose comprising at least about 5×10⁴ cells/kg, 5×10⁵ cells/kg, 5×10⁶ cells/kg, or up to about 5×10⁷ cells/kg. The number of cells will depend upon the ultimate use for which the composition is intended as will the type of cells included therein. For example, cells modified to contain a binding protein will comprise a cell population containing at least 30%, at least 35%, at least 40%, at least 45%, at least 50%, at least 55%, at least 60%, at least 65%, at least 70%, at least 75%, at least 80%, at least 85%, at least 90%, at least 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells. In certain embodiments, a unit dose of the modified immune cells can be co-administered with (e.g., simultaneously or contemporaneously with) hematopoietic stem cells from an allogeneic donor. In some embodiments, one or more of the modified immune cells comprised in the unit dose is autologous to the subject.

In some embodiments, the subject receiving the modified immune cell has previously received lymphodepleting chemotherapy. In further embodiments, the lymphodepleting chemotherapy comprises cyclophosphamide, fludarabine, anti-thymocyte globulin, or a combination thereof.

In some embodiments, the method further comprises administering an inhibitor of an immune checkpoint molecule, as disclosed herein, to the subject.

Also contemplated are pharmaceutical compositions (i.e., compositions) that comprise a composition (binding protein, polynucleotide, vector, host cell, host cell composition, unit dose, and/or immunogenic polypeptide) as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The pharmaceutical compositions described herein may be presented in unit-dose or multi-dose containers, such as sealed ampoules or vials. Such containers may be frozen to preserve the stability of the formulation until infusion into the patient. Doses will vary, but a dose for administration of a modified immune cell as described herein can be about 10⁴ cells/m², about 5×10⁴ cells/m², about 10⁵ cells/m², about 5×10⁵ cells/m², about 10⁶ cells/m², about 5×10⁶ cells/m², about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m². In certain embodiments, a unit dose comprises a modified immune cell as described herein at a dose of about 10⁴ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polyethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation can be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of engineered immune cells or active compound calculated to produce the desired effect in association with an appropriate pharmaceutical carrier.

In general, an appropriate dosage and treatment regimen provides the active molecules or cells in an amount sufficient to provide a benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine.

For prophylactic use, a dose can be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

As used herein, administration of a composition refers to delivering the same to a subject, regardless of the route or mode of delivery. Administration may be effected continuously or intermittently, and parenterally. A composition can be administered locally (e.g., intratumoral) or systemically (e.g., intravenously). Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., modified immune cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a composition described herein is administered to the subject, which may be administered at intervals between administrations of about two to about four weeks.

Treatment or prevention methods of this disclosure may be administered to a subject as part of a treatment course or regimen, which may comprise additional treatments prior to, or after, administration of the instantly disclosed unit doses, cells, or compositions. For example, in certain embodiments, a subject receiving a unit dose of the modified immune cell is receiving or had previously received a hematopoietic cell transplant (HCT; including myeloablative and non-myeloablative HCT). Techniques and regimens for performing HCT are known in the art and can comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in certain embodiments, a modified immune cell of the present disclosure can be administered with or shortly after hematopoietic stem cells in a modified HCT therapy. In some embodiments, the HCT comprises a donor hematopoietic cell comprising a chromosomal knockout of a gene that encodes an HLA component, a chromosomal knockout of a gene that encodes a TCR component, or both.

In further embodiments, the subject had previously received lymphodepleting chemotherapy prior to receiving the composition or HCT. In certain embodiments, a lymphodepleting chemotherapy comprises a conditioning regimen comprising cyclophosphamide, fludarabine, anti-thymocyte globulin, or a combination thereof.

Methods according to this disclosure may further include administering one or more additional agents to treat the disease or disorder in a combination therapy. For example, in certain embodiments, a combination therapy comprises administering a composition of the present disclosure with (concurrently, simultaneously, or sequentially) an immune checkpoint inhibitor. In some embodiments, a combination therapy comprises administering a composition of the present disclosure with an agonist of a stimulatory immune checkpoint agent. In further embodiments, a combination therapy comprises administering a composition of the present disclosure with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Example immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GAL9, KIR, PVR1G (CD112R), PVRL2, adenosine, A2aR, immunosuppressive cytokines (e.g., IL-10, IL-4, IL-1RA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise a composition of the present disclosure with one or more inhibitor of any one of the following immune suppression components, singly or in any combination.

In certain embodiments, a composition of the present disclosure is used in combination with a PD-1 inhibitor, for example a PD-1-specific antibody or binding fragment thereof, such as pidilizumab, nivolumab, pembrolizumab, MEDI0680 (formerly AMP-514), AMP-224, BMS-936558 or any combination thereof. In further embodiments, a composition of the present disclosure is used in combination with a PD-L1 specific antibody or binding fragment thereof, such as BMS-936559, durvalumab (MED14736), atezolizumab (RG7446), avelumab (MSB0010718C), MPDL3280A, or any combination thereof. Also contemplated are cemiplimab; IBI-308; nivolumab+relatlimab; BCD-100; camrelizumab; JS-001; spartalizumab; tislelizumab; AGEN-2034; BGBA-333+tislelizumab; CBT-501; dostarlimab; durvalumab+MEDI-0680; JNJ-3283; pazopanib hydrochloride+pembrolizumab; pidilizumab; REGN-1979+cemiplimab; ABBV-181; ADUS-100+spartalizumab; AK-104; AK-105; AMP-224; BAT-1306; BI-754091; CC-90006; cemiplimab+REGN-3767; CS-1003; GLS-010; LZM-009; MEDI-5752; MGD-013; PF-06801591; Sym-021; tislelizumab+pamiparib; XmAb-20717; AK-112; ALPN-202; AM-0001; an antibody to antagonize PD-1 for Alzheimer's disease; BH-2922; BH-2941; BH-2950; BH-2954; a biologic to antagonize CTLA-4 and PD-1 for solid tumor; a bispecific monoclonal antibody to target PD-1 and LAG-3 for oncology; BLSM-101; CB-201; CB-213; CBT-103; CBT-107; a cellular immunotherapy+PD-1 inhibitor; CX-188; HAB-21; HEISCOIII-003; IKT-202; JTX-4014; MCLA-134; MD-402; mDX-400; MGD-019; a monoclonal antibody to antagonize PDCD1 for oncology; a monoclonal antibody to antagonize PD-1 for oncology; an oncolytic virus to inhibit PD-1 for oncology; OT-2; PD-1 antagonist+ropeginterferon alfa-2b; PEGMP-7; PRS-332; RXI-762; STIA-1110; TSR-075; a vaccine to target HER2 and PD-1 for oncology; a vaccine to target PD-1 for oncology and autoimmune disorders; XmAb-23104; an antisense oligonucleotide to inhibit PD-1 for oncology; AT-16201; a bispecific monoclonal antibody to inhibit PD-1 for oncology; IMM-1802; monoclonal antibodies to antagonize PD-1 and CTLA-4 for solid tumor and hematological tumor; nivolumab biosimilar; a recombinant protein to agonize CD278 and CD28 and antagonize PD-1 for oncology; a recombinant protein to agonize PD-1 for autoimmune disorders and inflammatory disorders; SNA-01; SSI-361; YBL-006; AK-103; JY-034; AUR-012; BGB-108; drug to inhibit PD-1, Gal-9, and TIM-3 for solid tumor; ENUM-244C8; ENUM-388D4; MEDI-0680; monoclonal antibodies to antagonize PD-1 for metastatic melanoma and metastatic lung cancer; a monoclonal antibody to inhibit PD-1 for oncology; monoclonal antibodies to target CTLA-4 and PD-1 for oncology; a monoclonal antibody to antagonize PD-1 for NSCLC; monoclonal antibodies to inhibit PD-1 and TIM-3 for oncology; a monoclonal antibody to inhibit PD-1 for oncology; a recombinant protein to inhibit PD-1 and VEGF-A for hematological malignancies and solid tumor; a small molecule to antagonize PD-1 for oncology; Sym-016; inebilizumab+MEDI-0680; a vaccine to target PDL-1 and IDO for metastatic melanoma; an anti-PD-1 monoclonal antibody plus a cellular immunotherapy for glioblastoma; an antibody to antagonize PD-1 for oncology; monoclonal antibodies to inhibit PD-1/PD-L1 for hematological malignancies and bacterial infections; a monoclonal antibody to inhibit PD-1 for HIV; or a small molecule to inhibit PD-1 for solid tumor.

In certain embodiments, a composition of the present disclosure of the present disclosure is used in combination with a LAG3 inhibitor, such as LAG525, IMP321, IMP701, 9H12, BMS-986016, or any combination thereof.

In certain embodiments, a composition of the present disclosure is used in combination with an inhibitor of CTLA4. In particular embodiments, a composition of the present disclosure is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a composition of the present disclosure is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1.

In certain embodiments, a composition of the present disclosure is used in combination with an inhibitor of CD244.

In certain embodiments, a composition of the present disclosure is used in combination with an inhibitor of BLTA, HVEM, CD160, or any combination thereof. Anti CD160 antibodies are described in, for example, PCT Publication No. WO 2010/084158.

In certain embodiments, a composition of the present disclosure cell is used in combination with an inhibitor of TIM3.

In certain embodiments, a composition of the present disclosure is used in combination with an inhibitor of Gal9.

In certain embodiments, a composition of the present disclosure is used in combination with an inhibitor of adenosine signaling, such as a decoy adenosine receptor.

In certain embodiments, a composition of the present disclosure is used in combination with an inhibitor of A2aR.

In certain embodiments, a composition of the present disclosure is used in combination with an inhibitor of KIR, such as lirilumab (BMS-986015).

In certain embodiments, a composition of the present disclosure is used in combination with an inhibitor of an inhibitory cytokine (typically, a cytokine other than TGFβ) or Treg development or activity.

In certain embodiments, a composition of the present disclosure is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a composition of the present disclosure is used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof.

In certain embodiments, a composition of the present disclosure is used in combination with an inhibitor of VISTA, such as CA-170 (Curis, Lexington, Mass.).

In certain embodiments, a composition of the present disclosure is used in combination with an inhibitor of TIGIT such as, for example, COM902 (Compugen, Toronto, Ontario Canada), an inhibitor of CD155, such as, for example, COM701 (Compugen), or both.

In certain embodiments, a composition of the present disclosure is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a composition of the present disclosure is used in combination with a LAIR1 inhibitor.

In certain embodiments, a composition of the present disclosure is used in combination with an inhibitor of CEACAM-1, CEACAM-3, CEACAM-5, or any combination thereof.

In certain embodiments, a composition of the present disclosure is used in combination with an agent that increases the activity (i.e., is an agonist) of a stimulatory immune checkpoint molecule. For example a composition of the present disclosure can be used in combination with a CD137 (41BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a composition of the present disclosure with one or more agonist of a stimulatory immune checkpoint molecule, including any of the foregoing, singly or in any combination.

In certain embodiments, a combination therapy comprises a composition of the present disclosure and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a composition of the present disclosure and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer in a subject are well-known to those of ordinary skill in the art.

In certain embodiments, a combination therapy method comprises administering a composition of the present disclosure and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylprednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines may be used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with a composition of the present disclosure.

Also provided herein are methods for modulating an adoptive immunotherapy, wherein the methods comprise administering, to a subject who has previously received a modified host cell of the present disclosure that comprises a heterologous polynucleotide encoding a safety switch protein, a cognate compound of the safety switch protein in an amount effective to ablate in the subject the previously administered modified host cell.

In certain embodiments, the safety switch protein comprises tEGFR and the cognate compound is cetuximab, or the safety switch protein comprises iCasp9 and the cognate compound is AP1903 (e.g., dimerized AP1903), or the safety switch protein comprises a RQR polypeptide and the cognate compound is rituximab, or the safety switch protein comprises a myc binding domain and the cognate compound is an antibody specific for the myc binding domain.

In still further aspects, methods are provided for manufacturing a composition, or a unit dose of the present disclosure. In certain embodiments, the methods comprise combining (i) an aliquot of a host cell transduced with a vector of the present disclosure with (ii) a pharmaceutically acceptable carrier. In certain embodiments, vectors of the present disclosure are used to transfect/transduce a host cell (e.g., a T cell) for use in adoptive transfer therapy (e.g., targeting a cancer antigen).

In some embodiments, the methods further comprise, prior to the aliquoting, culturing the transduced host cell and selecting the transduced cell as having incorporated (i.e., expressing) the vector. In further embodiments, the methods comprise, following the culturing and selection and prior to the aliquoting, expanding the transduced host cell. In any of the embodiments of the instant methods, the manufactured composition or unit dose may be frozen (e.g., cryopreserved) for later use. Any appropriate host cell can be used for manufacturing a composition or unit dose according to the instant methods, including, for example, a hematopoietic stem cell, a T cell, a primary T cell, a T cell line, a NK cell, or a NK-T cell. In specific embodiments, the methods comprise a host cell which is a CD8⁺ T cell, a CD4⁺ T cell, or both.

Also provided are any of the binding proteins, polynucleotides, expression vectors, host cells, host cell compositions, unit doses, and immunogenic polypeptides, taken singly or in any combination, for use in treating a disease or disorder associated with a KRAS G12D mutation or a KRAS G12V or a NRAS G12D mutation or a NRAS G12V mutation or a HRAS G12V mutation or a HRAS G12D mutation in a subject.

Also provided are any of the binding proteins, polynucleotides, expression vectors, host cells, host cell compositions, unit doses, and immunogenic polypeptides, taken singly or in any combination, for use the manufacture of a medicament for treating a disease or disorder associated with a KRAS G12D mutation or a KRAS G12V or a NRAS G12D mutation or a NRAS G12V mutation or a HRAS G12V mutation or a HRAS G12D mutation in a subject.

In certain embodiments, the disease or disorder comprises a cancer. In some embodiments, the cancer is a solid cancer or a hematological malignancy. In certain embodiments, the disease or disorder is selected from a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Myelomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatosis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma. In some embodiments, the method comprises parenteral or intravenous administration of the subject composition. In some embodiments, the method comprises administering a plurality of doses of the binding protein, polynucleotide, expression vector, host cell, host cell composition, unit dose, and/or immunogenic polypeptide the subject.

In certain embodiments, the plurality of doses are administered at intervals between administrations of about two to about four weeks.

In certain embodiments, the composition comprises the modified host cell. In some embodiments, the method comprises administering the modified host cell to the subject at a dose of about 10⁴ cells/kg to about 10¹¹ cells/kg.

In certain embodiments, the method further comprises administering a cytokine to the subject. In some embodiments, the cytokine comprises IL-2, IL-15, or IL-21.

In certain embodiments, the subject has received or is receiving an immune checkpoint inhibitor and/or an agonist of a stimulatory immune checkpoint agent.

Also provided are methods that comprise introducing, into a host (e.g., T) cell, a polynucleotide encoding a binding protein of the present disclosure.

SEQUENCES

SEQ ID NO: 1-wt KRAS full (UniProt: P01116)
MTEYKLVVVG AGGVGKSALT IQLIQNHFVD EYDPTIEDSY RKQVVIDGET
CLLDILDTAG QEEYSAMRDQ YMRTGEGFLC VFAINNTKSF EDIHHYREQI
KRVKDSEDVP MVLVGNKCDL PSRTVDTKQA QDLARSYGIP FIETSAKTRQ
RVEDAFYTLV REIRQYRLKK ISKEEKTPGC VKIKKCIIM
SEQ ID NO: 2-KRAS 7-16 G12V
VVVGAVGVGK
SEQ ID NO: 3-KRAS 8-16 G12V
VVGAVGVGK
SEQ ID NO: 4-KRAS 8-16 G12V binding motif for TCR 11N4A
x-V-G-A-x-G-x-x-K
SEQ ID NO: 5-TCR 11N4A alpha chain with signal peptide-original (WT) nucleotide
sequence
atggccatgctcctgggggcatcagtgctgattctgtggcttcagccagactgggtaaacagtcaacagaagaatgatgaccagca
agttaagcaaaattcaccatccctgagcgtccaggaaggaagaatttctattctgaactgtgactatactaacagcatgtttgattat
ttcctatggtacaaaaaataccctgctgaaggtcctacattcctgatatctataagttccattaaggataaaaatgaagatggaagat
tcactgtcttcttaaacaaaagtgccaagcacctctctctgcacattgtgccctcccagcctggagactctgcagtgtacttctgtgca
gcaagtggggtttcaggaaacacacctcttgtctttggaaagggcacaagactttctgtgattgcaaatatccagaaccctgaccct
gccgtgtaccagctgagagactctaaatccagtgacaagtctgtctgcctattcaccgattttgattctcaaacaaatgtgtcacaaa
gtaaggattctgatgtgtatatcacagacaaaactgtgctagacatgaggtctatggacttcaagagcaacagtgctgtggcctgga
gcaacaaatctgactttgcatgtgcaaacgccttcaacaacagcattattccagaagacaccttcttccccagcccagaaagttcctg
tgatgtcaagctggtcgagaaaagctttgaaacagatacgaacctaaactttcaaaacctgtcagtgattgggttccgaatcctcctc
ctgaaagtggccgggtttaatctgctcatgacgctgcggctgtggtccagctga
SEQ ID NO: 6-TCR 11N4A beta chain with signal peptide-original (WT) nucleotide sequence
atgggctccaggctgctctgttgggtgctgctttgtctcctgggagcaggcccagtaaaggctggagtcactcaaactccaagatatc
tgatcaaaacgagaggacagcaagtgacactgagctgctcccctatctctgggcataggagtgtatcctggtaccaacagacccca
ggacagggccttcagttcctctttgaatacttcagtgagacacagagaaacaaaggaaacttccctggtcgattctcagggcgccag
ttctctaactctcgctctgagatgaatgtgagcaccttggagctgggggactcggccctttatctttgcgccagcagcgtcgggactgt
ggagcagtacttcgggccgggcaccaggctcacggtcacagaggacctgaaaaacgtgttcccacccgaggtcgctgtgtttgagc
catcagaagcagagatctcccacacccaaaaggccacactggtgtgcctggccacaggcttctaccccgaccacgtggagctgagc
tggtgggtgaatgggaaggaggtgcacagtggggtcagcacagacccgcagcccctcaaggagcagcccgccctcaatgactcca
gatactgcctgagcagccgcctgagggtctcggccaccttctggcagaacccccgcaaccacttccgctgtcaagtccagttctacg
ggctctcggagaatgacgagtggacccaggatagggccaaacctgtcacccagatcgtcagcgccgaggcctggggtagagcaga
ctgtggcttcacctccgagtcttaccagcaaggggtcctgtctgccaccatcctctatgagatcttgctagggaaggccaccttgtatg
ccgtgctggtcagtgccctcgtgctgatggccatggtcaagagaaaggattccagaggctag
SEQ ID NO: 7-TCR 11N4A TCRbeta-P2A-TCRalpha polynucleotide-Codon-optimization A
ATGGGCTCTAGACTGTTGTGTTGGGTTCTGCTGTGTCTGCTTGGAGCTGGACCTGTGAAAGCTGGAG
TTACCCAGACACCCAGATATCTGATCAAGACCAGAGGACAGCAGGTGACACTGAGCTGTAGCCCTAT
TTCTGGCCACAGGAGCGTTAGCTGGTATCAGCAAACACCCGGGCAGGGACTACAATTTCTATTCGAG
TACTTCAGCGAGACCCAGCGGAATAAGGGCAATTTTCCTGGCAGATTTAGCGGCAGGCAGTTCAGC
AACAGCAGAAGCGAGATGAACGTGAGCACCCTGGAATTAGGCGATTCTGCTCTGTACCTGTGTGCC
TCTTCTGTGGGAACAGTGGAGCAGTACTTTGGCCCCGGCACGAGACTGACAGTGACAGAGGACCTG
AAGAACGTGTTCCCCCCAGAGGTGGCCGTGTTCGAGCCTAGCGAGGCCGAGATCAGCCACACCCAG
AAAGCCACCCTCGTGTGCCTGGCCACCGGCTTTTACCCCGACCACGTGGAACTGTCTTGGTGGGTCA
ACGGCAAAGAGGTGCACAGCGGCGTCTGCACCGACCCCCAGCCCCTGAAAGAGCAGCCCGCCCTGA
ACGACAGCCGGTACTGTCTGAGCAGCAGACTGAGAGTGTCCGCCACCTTCTGGCAGAACCCCCGGA
ACCACTTCAGATGCCAGGTGCAGTTCTACGGCCTGAGCGAGAACGACGAGTGGACCCAGGACCGG
GCCAAGCCCGTGACCCAGATCGTGTCTGCTGAGGCCTGGGGCAGAGCCGATTGCGGCTTCACCAGC
GAGAGCTACCAGCAGGGCGTGCTGAGCGCCACCATCCTGTACGAGATCCTGCTGGGCAAGGCCACC
CTGTACGCCGTGCTGGTGTCCGCCCTGGTGCTGATGGCCATGGTCAAGCGGAAGGACAGCCGGGGC
GGTTCCGGAGCCACGAACTTCTCTCTGTTAAAGCAAGCAGGAGACGTGGAAGAAAACCCCGGTCCC
ATGGCCATGTTACTAGGAGCGAGCGTGCTGATTCTGTGGTTACAGCCTGATTGGGTGAACTCTCAGC
AGAAGAACGACGATCAGCAGGTGAAGCAGAATAGCCCCTCTCTGTCTGTGCAGGAGGGCAGAATCT
CTATCCTGAATTGCGACTACACCAACAGCATGTTCGACTATTTTCTGTGGTACAAAAAATACCCCGCC
GAGGGCCCTACATTCCTGATCAGCATCAGCTCTATCAAGGACAAGAACGAGGATGGCAGATTTACC
GTGTTCCTGAACAAGAGCGCCAAGCACCTGAGCCTGCACATTGTGCCTTCTCAACCTGGCGATTCTG
CTGTGTACTTTTGTGCTGCCTCTGGAGTGAGCGGCAATACACCTCTAGTGTTCGGGAAGGGCACAAG
ACTGTCTGTTATTGCAAACATTCAAAACCCCGACCCTGCTGTGTACCAGCTGCGGGACAGCAAGAGC
AGCGACAAGAGCGTGTGCCTGTTCACCGACTTCGACAGCCAGACCAACGTGTCCCAGAGCAAGGAC
AGCGACGTGTACATCACCGATAAGTGCGTGCTGGACATGCGGAGCATGGACTTCAAGAGCAACAGC
GCCGTGGCCTGGTCCAACAAGAGCGACTTCGCCTGCGCCAACGCCTTCAACAACAGCATTATCCCCG
AGGACACATTCTTCCCAAGCCCCGAGAGCAGCTGCGACGTGAAGCTGGTGGAAAAGAGCTTCGAGA
CAGACACCAACCTGAACTTCCAGAACCTCAGCGTGATCGGCTTCCGGATCCTGCTGCTGAAGGTGGC
CGGCTTCAACCTGCTGATGACCCTGCGGCTGTGGTCCAGCTGA
SEQ ID NO: 8-11N4A TCRbeta-P2A-alpha polynucleotide Codon-optimization B
ATGGGATCTAGATTGCTTTGTTGGGTGCTGCTGTGCCTGCTCGGAGCCGGACCTGTGAAAGCTGGC
GTTACCCAGACACCTAGATACCTGATCAAGACCAGAGGCCAGCAAGTGACCCTGAGCTGCTCTCCTA
TCAGCGGCCACAGAAGCGTGTCCTGGTATCAGCAGACACCTGGACAGGGCCTGCAGTTCCTGTTCG
AGTACTTCAGCGAGACACAGCGGAACAAGGGCAACTTCCCCGGCAGATTTTCCGGCAGACAGTTCA
GCAACAGCCGCAGCGAGATGAACGTGTCCACACTGGAACTGGGCGACAGCGCCCTGTATCTGTGTG
CCTCTTCTGTGGGCACCGTGGAACAGTACTTTGGCCCTGGCACCAGACTGACCGTGACCGAGGATCT
GAAGAACGTGTTCCCACCTGAGGTGGCCGTGTTCGAGCCTTCTGAGGCCGAGATCAGCCACACACA
GAAAGCCACACTCGTGTGTCTGGCCACCGGCTTCTATCCCGATCACGTGGAACTGTCTTGGTGGGTC
AACGGCAAAGAGGTGCACAGCGGCGTCTGTACCGATCCTCAGCCTCTGAAAGAGCAGCCCGCTCTG
AACGACAGCAGATACTGCCTGAGCAGCAGACTGAGAGTGTCCGCCACCTTCTGGCAGAACCCCAGA
AACCACTTCAGATGCCAGGTGCAGTTCTACGGCCTGAGCGAGAACGATGAGTGGACCCAGGATAGA
GCCAAGCCTGTGACACAGATCGTGTCTGCCGAAGCCTGGGGCAGAGCCGATTGTGGCTTTACCAGC
GAGAGCTACCAGCAGGGCGTGCTGTCTGCCACAATCCTGTACGAGATCCTGCTGGGCAAAGCCACT
CTGTACGCCGTGCTGGTTTCTGCCCTGGTGCTGATGGCCATGGTCAAGCGGAAGGATTCTAGAGGC
GGATCCGGAGCCACCAACTTCAGCCTGCTTAAACAGGCCGGCGACGTGGAAGAGAACCCTGGACCT
ATGGCTATGCTGCTGGGAGCCTCTGTGCTGATCCTGTGGCTGCAACCCGATTGGGTCAACAGCCAGC
AGAAGAACGACGACCAGCAAGTCAAGCAGAACAGCCCCAGCCTGAGCGTGCAAGAGGGCAGAATC
AGCATCCTGAACTGCGACTACACCAACTCTATGTTCGACTACTTTCTGTGGTACAAGAAGTACCCCGC
CGAGGGACCCACCTTCCTGATCAGCATCAGCAGCATCAAGGACAAGAACGAGGACGGCCGGTTCAC
CGTGTTTCTGAACAAGAGCGCCAAGCACCTGAGCCTGCACATCGTGCCTTCTCAGCCTGGCGATAGC
GCCGTGTACTTTTGTGCTGCCAGCGGCGTGTCAGGCAACACCCCTCTGGTTTTTGGCAAGGGCACAC
GCCTGTCCGTGATCGCCAACATTCAGAACCCTGATCCTGCCGTGTACCAGCTGAGAGACAGCAAGAG
CAGCGACAAGAGCGTGTGCCTGTTCACCGACTTCGACAGCCAGACCAACGTGTCCCAGAGCAAGGA
CAGCGACGTGTACATCACCGATAAGTGCGTGCTGGACATGCGGAGCATGGACTTCAAGAGCAACAG
CGCCGTGGCCTGGTCCAACAAGTCCGATTTCGCCTGCGCCAACGCCTTCAACAACAGCATTATCCCC
GAGGACACATTCTTCCCAAGTCCTGAGTCCAGCTGCGACGTGAAGCTGGTGGAAAAGAGCTTCGAG
ACAGACACCAACCTGAACTTCCAGAATCTGAGCGTGATCGGCTTCAGAATCCTGCTGCTGAAGGTGG
CCGGATTCAACCTGCTGATGACCCTCAGACTGTGGTCCAGCTGA
SEQ ID NO: 9-CD8alpha-T2A-CD8beta-P2A-11N4A TCRbeta-P2A-alpha
polynucleotide Codon-optimization A
ATGGCTCTGCCTGTGACAGCTCTGCTGCTGCCTCTGGCTCTGCTTCTGCATGCCGCTAGACCCAGCCA
GTTCAGAGTGTCCCCTCTGGACAGAACCTGGAACCTGGGCGAGACAGTGGAACTGAAGTGCCAGGT
GCTGCTGAGCAATCCTACCAGCGGCTGCAGCTGGCTGTTTCAGCCTAGAGGTGCTGCCGCCTCTCCT
ACCTTTCTGCTGTACCTGAGCCAGAACAAGCCCAAGGCCGCCGAAGGACTGGACACCCAGAGATTC
AGCGGCAAGAGACTGGGCGACACCTTCGTGCTGACCCTGAGCGACTTCAGAAGAGAGAACGAGGG
CTACTACTTCTGCAGCGCCCTGAGCAACAGCATCATGTACTTCAGCCACTTCGTGCCCGTGTTTCTGC
CCGCCAAGCCTACAACAACCCCTGCTCCTAGACCTCCTACACCAGCTCCTACAATCGCCAGCCAGCCT
CTGTCTCTGAGGCCAGAAGCTTGTAGACCTGCTGCTGGCGGAGCCGTGCATACAAGAGGACTGGAT
TTCGCCTGCGACATCTACATCTGGGCCCCTCTGGCTGGAACATGTGGCGTGCTGCTGCTGTCCCTGG
TCATCACCCTGTACTGCAACCACCGGAACAGGCGGAGAGTGTGCAAGTGCCCTAGACCTGTGGTCA
AGAGCGGCGACAAGCCTAGCCTGAGCGCCAGATATGTTGGCAGCGGAGAAGGCAGAGGCTCCCTG
CTTACATGCGGCGACGTGGAAGAGAACCCCGGACCTATGAGGCCTAGACTGTGGCTGCTTCTGGCT
GCCCAGCTGACAGTGCTGCACGGCAATTCTGTCCTGCAGCAGACCCCTGCCTACATCAAGGTGCAGA
CCAACAAGATGGTCATGCTGAGCTGCGAGGCCAAGATCAGCCTGTCCAACATGCGGATCTACTGGC
TGCGGCAGAGACAGGCCCCTAGCTCTGATAGCCACCACGAGTTTCTGGCCCTGTGGGATTCTGCCAA
GGGCACCATTCACGGCGAGGAAGTGGAACAAGAGAAGATCGCCGTGTTCCGGGACGCCAGCAGAT
TCATCCTGAACCTGACCAGCGTGAAGCCCGAGGACAGCGGCATCTATTTCTGCATGATCGTGGGCA
GCCCCGAGCTGACATTTGGCAAGGGAACACAGCTGAGCGTGGTGGACTTCCTGCCTACTACAGCCC
AGCCTACCAAGAAGTCTACCCTGAAGAAACGCGTGTGCAGACTGCCCAGGCCTGAGACACAAAAGG
GCCCTCTGTGCAGCCCTATCACACTGGGATTGCTGGTGGCTGGCGTTCTGGTCCTGCTGGTGTCTCT
GGGAGTTGCCATCCACCTGTGCTGTAGAAGAAGGCGGGCCAGACTGCGGTTCATGAAGCAGTTCTA
CAAAGGCAGCGGCGCCACCAACTTCAGCCTGCTGAAACAAGCCGGCGACGTCGAGGAAAATCCTGG
ACCTATGGGCTCTAGACTGTTGTGTTGGGTTCTGCTGTGTCTGCTTGGAGCTGGACCTGTGAAAGCT
GGAGTTACCCAGACACCCAGATATCTGATCAAGACCAGAGGACAGCAGGTGACACTGAGCTGTAGC
CCTATTTCTGGCCACAGGAGCGTTAGCTGGTATCAGCAAACACCCGGGCAGGGACTACAATTTCTAT
TCGAGTACTTCAGCGAGACCCAGCGGAATAAGGGCAATTTTCCTGGCAGATTTAGCGGCAGGCAGT
TCAGCAACAGCAGAAGCGAGATGAACGTGAGCACCCTGGAATTAGGCGATTCTGCTCTGTACCTGT
GTGCCTCTTCTGTGGGAACAGTGGAGCAGTACTTTGGCCCCGGCACGAGACTGACAGTGACAGAGG
ACCTGAAGAACGTGTTCCCCCCAGAGGTGGCCGTGTTCGAGCCTAGCGAGGCCGAGATCAGCCACA
CCCAGAAAGCCACCCTCGTGTGCCTGGCCACCGGCTTTTACCCCGACCACGTGGAACTGTCTTGGTG
GGTCAACGGCAAAGAGGTGCACAGCGGCGTCTGCACCGACCCCCAGCCCCTGAAAGAGCAGCCCG
CCCTGAACGACAGCCGGTACTGTCTGAGCAGCAGACTGAGAGTGTCCGCCACCTTCTGGCAGAACC
CCCGGAACCACTTCAGATGCCAGGTGCAGTTCTACGGCCTGAGCGAGAACGACGAGTGGACCCAGG
ACCGGGCCAAGCCCGTGACCCAGATCGTGTCTGCTGAGGCCTGGGGCAGAGCCGATTGCGGCTTCA
CCAGCGAGAGCTACCAGCAGGGCGTGCTGAGCGCCACCATCCTGTACGAGATCCTGCTGGGCAAGG
CCACCCTGTACGCCGTGCTGGTGTCCGCCCTGGTGCTGATGGCCATGGTCAAGCGGAAGGACAGCC
GGGGCGGTTCCGGAGCCACGAACTTCTCTCTGTTAAAGCAAGCAGGAGACGTGGAAGAAAACCCCG
GTCCCATGGCCATGTTACTAGGAGCGAGCGTGCTGATTCTGTGGTTACAGCCTGATTGGGTGAACTC
TCAGCAGAAGAACGACGATCAGCAGGTGAAGCAGAATAGCCCCTCTCTGTCTGTGCAGGAGGGCA
GAATCTCTATCCTGAATTGCGACTACACCAACAGCATGTTCGACTATTTTCTGTGGTACAAAAAATAC
CCCGCCGAGGGCCCTACATTCCTGATCAGCATCAGCTCTATCAAGGACAAGAACGAGGATGGCAGA
TTTACCGTGTTCCTGAACAAGAGCGCCAAGCACCTGAGCCTGCACATTGTGCCTTCTCAACCTGGCG
ATTCTGCTGTGTACTTTTGTGCTGCCTCTGGAGTGAGCGGCAATACACCTCTAGTGTTCGGGAAGGG
CACAAGACTGTCTGTTATTGCAAACATTCAAAACCCCGACCCTGCTGTGTACCAGCTGCGGGACAGC
AAGAGCAGCGACAAGAGCGTGTGCCTGTTCACCGACTTCGACAGCCAGACCAACGTGTCCCAGAGC
AAGGACAGCGACGTGTACATCACCGATAAGTGCGTGCTGGACATGCGGAGCATGGACTTCAAGAGC
AACAGCGCCGTGGCCTGGTCCAACAAGAGCGACTTCGCCTGCGCCAACGCCTTCAACAACAGCATTA
TCCCCGAGGACACATTCTTCCCAAGCCCCGAGAGCAGCTGCGACGTGAAGCTGGTGGAAAAGAGCT
TCGAGACAGACACCAACCTGAACTTCCAGAACCTCAGCGTGATCGGCTTCCGGATCCTGCTGCTGAA
GGTGGCCGGCTTCAACCTGCTGATGACCCTGCGGCTGTGGTCCAGCTGA
SEQ ID NO: 10-CD8alpha-T2A-CD8beta-P2A-11N4A TCRbeta-P2A-alpha
polynucleotide Codon-optimization B
ATGGCATTGCCTGTTACAGCTCTGCTGCTGCCCCTGGCTCTGCTTCTGCATGCTGCTAGACCCAGCCA
GTTCAGAGTGTCCCCTCTGGACAGAACCTGGAACCTGGGCGAGACAGTGGAACTGAAGTGCCAGGT
GCTGCTGAGCAATCCTACCAGCGGCTGCAGCTGGCTGTTTCAGCCTAGAGGTGCTGCCGCCTCTCCT
ACCTTTCTGCTGTACCTGAGCCAGAACAAGCCCAAGGCCGCCGAAGGACTGGACACCCAGAGATTC
AGCGGCAAGAGACTGGGCGACACCTTCGTGCTGACCCTGAGCGACTTCAGAAGAGAGAACGAGGG
CTACTACTTCTGCAGCGCCCTGAGCAACAGCATCATGTACTTCAGCCACTTCGTGCCCGTGTTTCTGC
CCGCCAAGCCTACAACAACCCCTGCTCCTAGACCTCCTACACCAGCTCCTACAATCGCCAGCCAGCCT
CTGTCTCTGAGGCCAGAAGCTTGTAGACCTGCTGCTGGCGGAGCCGTGCATACAAGAGGACTGGAT
TTCGCCTGCGACATCTACATCTGGGCCCCTCTGGCTGGAACATGTGGCGTGCTGCTGCTGTCTCTGG
TCATCACCCTGTACTGCAACCACCGGAACAGGCGGAGAGTGTGCAAGTGCCCTAGACCTGTGGTCA
AGAGCGGCGACAAGCCTAGCCTGAGCGCCAGATATGTTGGCAGCGGAGAAGGCAGAGGCAGCCTG
CTTACATGCGGCGACGTGGAAGAGAACCCCGGACCTATGAGGCCTAGACTGTGGCTGCTTCTGGCT
GCCCAGCTGACAGTGCTGCACGGCAATTCTGTCCTGCAGCAGACCCCTGCCTACATCAAGGTGCAGA
CCAACAAGATGGTCATGCTGAGCTGCGAGGCCAAGATCAGCCTGTCCAACATGCGGATCTACTGGC
TGCGGCAGAGACAGGCCCCTAGCAGCGATTCTCACCACGAGTTTCTGGCCCTGTGGGATAGCGCCA
AGGGAACCATTCACGGCGAGGAAGTGGAACAAGAGAAGATCGCCGTGTTCCGGGACGCCAGCAGA
TTCATCCTGAACCTGACCAGCGTGAAGCCCGAGGACAGCGGCATCTATTTCTGCATGATCGTGGGCA
GCCCCGAGCTGACATTTGGCAAGGGAACACAGCTGAGCGTGGTGGACTTCCTGCCTACTACAGCCC
AGCCTACCAAGAAGTCTACCCTGAAGAAACGCGTGTGCAGACTGCCCAGGCCTGAGACACAAAAGG
GCCCTCTGTGCAGCCCTATCACACTGGGATTGCTGGTGGCTGGCGTTCTGGTCCTGCTGGTTTCTCTG
GGAGTTGCCATCCACCTGTGCTGCAGACGCAGAAGGGCCAGACTGCGGTTCATGAAGCAGTTCTAC
AAAGGCAGCGGCGCCACCAACTTCAGCCTGCTGAAACAAGCCGGCGACGTCGAAGAAAATCCTGGA
CCAATGGGCAGCAGACTGCTGTGCTGGGTTCTGCTGTGTCTGCTTGGAGCCGGACCTGTGAAAGCT
GGCGTGACCCAGACACCTAGATACCTGATCAAGACCAGAGGCCAGCAAGTGACACTGAGCTGTAGC
CCCATCAGCGGCCACAGAAGCGTGTCCTGGTATCAGCAGACTCCTGGACAGGGCCTGCAGTTCCTGT
TCGAGTACTTCTCCGAGACACAGAGGAACAAGGGCAACTTCCCCGGCAGATTCTCCGGCAGACAGT
TCAGCAACTCCCGCAGCGAGATGAACGTGTCCACACTGGAACTGGGAGATAGCGCCCTGTACCTGT
GTGCCTCTTCTGTGGGAACCGTGGAACAGTACTTCGGCCCTGGCACAAGACTGACCGTGACCGAGG
ACCTGAAGAACGTGTTCCCACCTGAGGTGGCCGTGTTCGAGCCTTCTGAGGCCGAGATCTCTCACAC
CCAGAAAGCCACACTCGTGTGTCTGGCCACCGGCTTCTATCCCGATCACGTGGAACTGTCTTGGTGG
GTCAACGGCAAAGAGGTGCACAGCGGCGTCTGTACCGATCCTCAGCCACTGAAAGAGCAGCCCGCT
CTGAACGACAGCAGATACTGCCTGTCCTCCAGACTGAGAGTGTCCGCCACCTTCTGGCAGAACCCCA
GAAACCACTTCAGGTGTCAGGTGCAGTTTTACGGCCTGAGCGAGAACGACGAGTGGACCCAGGATA
GAGCCAAGCCTGTGACACAGATCGTGTCTGCCGAAGCCTGGGGCAGAGCCGATTGTGGCTTTACCA
GCGAGAGCTACCAGCAGGGCGTTCTGTCTGCCACCATCCTGTACGAGATCCTGCTGGGCAAAGCCA
CTCTGTACGCCGTGTTGGTGTCTGCCCTGGTGCTGATGGCCATGGTCAAGCGGAAGGATTCTAGAG
GCGGATCCGGAGCCACAAATTTCTCACTGCTGAAGCAGGCCGGGGATGTTGAGGAAAACCCAGGAC
CTATGGCTATGCTGCTGGGAGCCTCTGTGCTGATCCTGTGGCTGCAACCCGATTGGGTCAACAGCCA
GCAGAAGAACGACGACCAGCAAGTCAAGCAGAACAGCCCCAGCCTGAGCGTGCAAGAGGGCAGAA
TCAGCATCCTGAACTGCGACTACACCAACTCTATGTTCGACTACTTTCTGTGGTACAAGAAGTACCCC
GCCGAGGGACCCACCTTCCTGATCAGCATCAGCAGCATCAAGGACAAGAACGAGGACGGCCGGTTC
ACCGTGTTTCTGAACAAGAGCGCCAAGCACCTGAGCCTGCACATCGTGCCTTCTCAGCCTGGCGATA
GCGCCGTGTACTTTTGTGCTGCCAGCGGCGTGTCAGGCAACACCCCTCTGGTTTTTGGCAAGGGCAC
ACGCCTGTCCGTGATCGCCAACATTCAGAACCCTGATCCTGCCGTGTACCAGCTGAGAGACAGCAAG
AGCAGCGACAAGAGCGTGTGCCTGTTCACCGACTTCGACAGCCAGACCAACGTGTCCCAGAGCAAG
GACAGCGACGTGTACATCACCGATAAGTGCGTGCTGGACATGCGGAGCATGGACTTCAAGAGCAAC
AGCGCCGTGGCCTGGTCCAACAAGTCCGATTTCGCCTGCGCCAACGCCTTCAACAACAGCATTATCC
CCGAGGACACATTCTTCCCAAGTCCTGAGTCCAGCTGCGACGTGAAGCTGGTGGAAAAGAGCTTCG
AGACAGACACCAACCTGAACTTCCAGAATCTGAGCGTGATCGGCTTCAGAATCCTGCTGCTGAAGGT
GGCCGGATTCAACCTGCTGATGACCCTCAGACTGTGGTCCAGCTGA
SEQ ID NO: 11-11N4A TCR alpha chain-original protein, with signal peptide underlined
MAMLLGASVLILWLQPDWVNSQQKNDDQQVKQNSPSLSVQEGRISILNCDYTNSMFDYFLWYKKYPA
EGPTFLISISSIKDKNEDGRFTVFLNKSAKHLSLHIVPSQPGDSAVYFCAASGVSGNTPLVFGKGTRLSVIAN
IQNPDPAVYQLRDSKSSDKSVCLFTDFDSQTNVSQSKDSDVYITDKTVLDMRSMDFKSNSAVAWSNKSD
FACANAFNNSIIPEDTFFPSPESSCDVKLVEKSFETDTNLNFQNLSVIGFRILLLKVAGFNLLMTLRLWSS*
SEQ ID NO: 12-11N4A TCR alpha chain-original protein, without signal peptide
QQKNDDQQVKQNSPSLSVQEGRISILNCDYTNSMFDYFLWYKKYPAEGPTFLISISSIKDKNEDGRFTVFL
NKSAKHLSLHIVPSQPGDSAVYFCAASGVSGNTPLVFGKGTRLSVIANIQNPDPAVYQLRDSKSSDKSVCL
FTDFDSQTNVSQSKDSDVYITDKTVLDMRSMDFKSNSAVAWSNKSDFACANAFNNSIIPEDTFFPSPESS
CDVKLVEKSFETDTNLNFQNLSVIGFRILLLKVAGFNLLMTLRLWSS*
SEQ ID NO: 13-11N4A TCR alpha chain variable domain, without signal peptide
QQKNDDQQVKQNSPSLSVQEGRISILNCDYTNSMFDYFLWYKKYPAEGPTFLISISSIKDKNEDGRFTVFL
NKSAKHLSLHIVPSQPGDSAVYFCAASGVSGNTPLVFGKGTRLSVIA
SEQ ID NO: 14-11N4A TCR alpha chain variable domain CDR1α
NSMFDY
SEQ ID NO: 15-11N4A TCR alpha chain variable domain CDR2α
ISSIKDK
SEQ ID NO: 16-11N4A TCR alpha chain variable domain CDR3α-IMGT junction
CAASGVSGNTPLVF
SEQ ID NO: 17-11N4A TCR alpha chain variable domain CDR3α-IMGT
AASGVSGNTPLV
SEQ ID NO: 18-11N4A TCR alpha chain constant domain (original protein)
NIQNPDPAVYQLRDSKSSDKSVCLFTDFDSQTNVSQSKDSDVYITDKTVLDMRSMDFKSNSAVAWSNK
SDFACANAFNNSIIPEDTFFPSPESSCDVKLVEKSFETDTNLNFQNLSVIGFRILLLKVAGFNLLMTLRLWSS
SEQ ID NO: 19-11N4A TCR alpha chain constant domain (cys-modified protein)
NIQNPDPAVYQLRDSKSSDKSVCLFTDFDSQTNVSQSKDSDVYITDKCVLDMRSMDFKSNSAVAWSNK
SDFACANAFNNSIIPEDTFFPSPESSCDVKLVEKSFETDTNLNFQNLSVIGFRILLLKVAGFNLLMTLRLWSS
SEQ ID NO: 20-11N4A TCR alpha chain, without signal peptide, cys-modified
QQKNDDQQVKQNSPSLSVQEGRISILNCDYTNSMFDYFLWYKKYPAEGPTFLISISSIKDKNEDGRFTVFL
NKSAKHLSLHIVPSQPGDSAVYFCAASGVSGNTPLVFGKGTRLSVIANIQNPDPAVYQLRDSKSSDKSVCL
FTDFDSQTNVSQSKDSDVYITDKCVLDMRSMDFKSNSAVAWSNKSDFACANAFNNSIIPEDTFFPSPESS
CDVKLVEKSFETDTNLNFQNLSVIGFRILLLKVAGFNLLMTLRLWSS
SEQ ID NO: 21-11N4A TCR beta chain-original protein, with signal peptide underlined
MGSRLLCWVLLCLLGAGPVKAGVTQTPRYLIKTRGQQVTLSCSPISGHRSVSWYQQTPGQGLQFLFEYFS
ETQRNKGNFPGRFSGRQFSNSRSEMNVSTLELGDSALYLCASSVGTVEQYFGPGTRLTVTEDLKNVFPPE
VAVFEPSEAEISHTQKATLVCLATGFYPDHVELSWWVNGKEVHSGVSTDPQPLKEQPALNDSRYCLSSRL
RVSATFWQNPRNHFRCQVQFYGLSENDEWTQDRAKPVTQIVSAEAWGRADCGFTSESYQQGVLSATI
LYEILLGKATLYAVLVSALVLMAMVKRKDSRG
SEQ ID NO: 22-11N4A TCR beta chain-original protein, without signal peptide
GVTQTPRYLIKTRGQQVTLSCSPISGHRSVSWYQQTPGQGLQFLFEYFSETQRNKGNFPGRFSGRQFSNS
RSEMNVSTLELGDSALYLCASSVGTVEQYFGPGTRLTVTEDLKNVFPPEVAVFEPSEAEISHTQKATLVCL
ATGFYPDHVELSWWVNGKEVHSGVSTDPQPLKEQPALNDSRYCLSSRLRVSATFWQNPRNHFRCQVQ
FYGLSENDEWTQDRAKPVTQIVSAEAWGRADCGFTSESYQQGVLSATILYEILLGKATLYAVLVSALVLM
AMVKRKDSRG
SEQ ID NO: 23-11N4A TCR beta chain variable domain, without signal peptide
GVTQTPRYLIKTRGQQVTLSCSPISGHRSVSWYQQTPGQGLQFLFEYFSETQRNKGNFPGRFSGRQFSNS
RSEMNVSTLELGDSALYLCASSVGTVEQYFGPGTRLTVT
SEQ ID NO: 24-11N4A TCR beta chain variable domain CDR1ß
SGHRS
SEQ ID NO: 25-11N4A TCR beta chain variable domain CDR2ß
YFSETQ
SEQ ID NO: 26-11N4A TCR beta chain variable domain CDR3ß-IMGT junction
CASSVGTVEQYF
SEQ ID NO: 27-11N4A TCR beta chain variable domain CDR3ß-IMGT
ASSVGTVEQY
SEQ ID NO: 28-11N4A TCR beta chain constant domain (original protein)
EDLKNVFPPEVAVFEPSEAEISHTQKATLVCLATGFYPDHVELSWWVNGKEVHSGVSTDPQPLKEQPAL
NDSRYCLSSRLRVSATFWQNPRNHFRCQVQFYGLSENDEWTQDRAKPVTQIVSAEAWGRADCGFTSE
SYQQGVLSATILYEILLGKATLYAVLVSALVLMAMVKRKDSRG*
SEQ ID NO: 29-11N4A TCR beta chain constant domain (cys-modified protein)
EDLKNVFPPEVAVFEPSEAEISHTQKATLVCLATGFYPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCGFTSE
SYQQGVLSATILYEILLGKATLYAVLVSALVLMAMVKRKDSRG
SEQ ID NO: 30-11N4A TCR beta chain, without signal peptide (cys-modified protein)
GVTQTPRYLIKTRGQQVTLSCSPISGHRSVSWYQQTPGQGLQFLFEYFSETQRNKGNFPGRFSGRQFSNS
RSEMNVSTLELGDSALYLCASSVGTVEQYFGPGTRLTVTEDLKNVFPPEVAVFEPSEAEISHTQKATLVCL
ATGFYPDHVELSWWVNGKEVHSGVCTDPQPLKEQPALNDSRYCLSSRLRVSATFWQNPRNHFRCQVQ
FYGLSENDEWTQDRAKPVTQIVSAEAWGRADCGFTSESYQQGVLSATILYEILLGKATLYAVLVSALVLM
AMVKRKDSRG
SEQ ID NO: 31-11N4A TCRbeta-P2A-alpha-Protein, with signal peptides underlined
MGSRLLCWVLLCLLGAGPVKAGVTQTPRYLIKTRGQQVTLSCSPISGHRSVSWYQQTPGQGLQFLFEYFS
ETQRNKGNFPGRFSGRQFSNSRSEMNVSTLELGDSALYLCASSVGTVEQYFGPGTRLTVTEDLKNVFPPE
VAVFEPSEAEISHTQKATLVCLATGFYPDHVELSWWVNGKEVHSGVCTDPQPLKEQPALNDSRYCLSSRL
RVSATFWQNPRNHFRCQVQFYGLSENDEWTQDRAKPVTQIVSAEAWGRADCGFTSESYQQGVLSATI
LYEILLGKATLYAVLVSALVLMAMVKRKDSRGGSGATNFSLLKQAGDVEENPGPMAMLLGASVLILWLQ
PDWVNSQQKNDDQQVKQNSPSLSVQEGRISILNCDYTNSMFDYFLWYKKYPAEGPTFLISISSIKDKNED
GRFTVFLNKSAKHLSLHIVPSQPGDSAVYFCAASGVSGNTPLVFGKGTRLSVIANIQNPDPAVYQLRDSKS
SDKSVCLFTDFDSQTNVSQSKDSDVYITDKCVLDMRSMDFKSNSAVAWSNKSDFACANAFNNSIIPEDT
FFPSPESSCDVKLVEKSFETDTNLNFQNLSVIGFRILLLKVAGFNLLMTLRLWSS*
SEQ ID NO: 32 CD8alpha-T2A-CD8beta-P2A-11N4A TCRbeta-P2A-alpha-Protein,
with signal peptides underlined
MALPVTALLLPLALLLHAARPSQFRVSPLDRTWNLGETVELKCQVLLSNPTSGCSWLFQPRGAAASPTFL
LYLSQNKPKAAEGLDTQRFSGKRLGDTFVLTLSDFRRENEGYYFCSALSNSIMYFSHFVPVFLPAKPTTTPA
PRPPTPAPTIASQPLSLRPEACRPAAGGAVHTRGLDFACDIYIWAPLAGTCGVLLLSLVITLYCNHRNRRR
VCKCPRPVVKSGDKPSLSARYVGSGEGRGSLLTCGDVEENPGPMRPRLWLLLAAQLTVLHGNSVLQQTP
AYIKVQTNKMVMLSCEAKISLSNMRIYWLRQRQAPSSDSHHEFLALWDSAKGTIHGEEVEQEKIAVFRD
ASRFILNLTSVKPEDSGIYFCMIVGSPELTFGKGTQLSVVDFLPTTAQPTKKSTLKKRVCRLPRPETQKGPLC
SPITLGLLVAGVLVLLVSLGVAIHLCCRRRRARLRFMKQFYKGSGATNFSLLKQAGDVEENPGPMGSRLLC
WVLLCLLGAGPVKAGVTQTPRYLIKTRGQQVTLSCSPISGHRSVSWYQQTPGQGLQFLFEYFSETQRNK
GNFPGRFSGRQFSNSRSEMNVSTLELGDSALYLCASSVGTVEQYFGPGTRLTVTEDLKNVFPPEVAVFEP
SEAEISHTQKATLVCLATGFYPDHVELSWWVNGKEVHSGVCTDPQPLKEQPALNDSRYCLSSRLRVSATF
WQNPRNHFRCQVQFYGLSENDEWTQDRAKPVTQIVSAEAWGRADCGFTSESYQQGVLSATILYEILLG
KATLYAVLVSALVLMAMVKRKDSRGGSGATNFSLLKQAGDVEENPGPMAMLLGASVLILWLQPDWVN
SQQKNDDQQVKQNSPSLSVQEGRISILNCDYTNSMFDYFLWYKKYPAEGPTFLISISSIKDKNEDGRFTVF
LNKSAKHLSLHIVPSQPGDSAVYFCAASGVSGNTPLVFGKGTRLSVIANIQNPDPAVYQLRDSKSSDKSVC
LFTDFDSQTNVSQSKDSDVYITDKCVLDMRSMDFKSNSAVAWSNKSDFACANAFNNSIIPEDTFFPSPES
SCDVKLVEKSFETDTNLNFQNLSVIGFRILLLKVAGFNLLMTLRLWSS*
SEQ ID NO: 33-11N6 TCR alpha-original nucleotide sequence
Atggaaactctcctgggagtgtctttggtgattctatggcttcaactggctagggtgaacagtcaacagggagaagaggatcctcag
gccttgagcatccaggagggtgaaaatgccaccatgaactgcagttacaaaactagtataaacaatttacagtggtatagacaaaa
ttcaggtagaggccttgtccacctaattttaatacgttcaaatgaaagagagaaacacagtggaagattaagagtcacgcttgacac
ttccaagaaaagcagttccttgttgatcacggcttcccgggcagcagacactgcttcttacttctgtgctacggaccctatgaacacc
aatgcaggcaaatcaacctttggggatgggactacgctcactgtgaagccaaatatccagaaccctgaccctgccgtgtaccagct
gagagactctaaatccagtgacaagtctgtctgcctattcaccgattttgattctcaaacaaatgtgtcacaaagtaaggattctgat
gtgtatatcacagacaaaactgtgctagacatgaggtctatggacttcaagagcaacagtgctgtggcctggagcaacaaatctga
ctttgcatgtgcaaacgccttcaacaacagcattattccagaagacaccttcttccccagcccagaaagttcctgtgatgtcaagctg
gtcgagaaaagctttgaaacagatacgaacctaaactttcaaaacctgtcagtgattgggttccgaatcctcctcctgaaagtggcc
gggtttaatctgctcatgacgctgcggctgtggtccagctga
SEQ ID NO: 34--11N6 TCR beta-original nucleotide sequence
atgggcaccaggctcctctgctgggcggccctctgtctcctgggagcagaactcacagaagctggagttgcccagtctcccagatat
aagattatagagaaaaggcagagtgtggctttttggtgcaatcctatatctggccatgctaccctttactggtaccagcagatcctgg
gacagggcccaaagcttctgattcagtttcagaataacggtgtagtggatgattcacagttgcctaaggatcgattttctgcagagag
gctcaaaggagtagactccactctcaagatccaacctgcaaagcttgaggactcggccgtgtatctctgtgccagcagcccctacgg
ggggagcgtctcctacgagcagtacttcgggccgggcaccaggctcacggtcacagaggacctgaaaaacgtgttcccacccgag
gtcgctgtgtttgagccatcagaagcagagatctcccacacccaaaaggccacactggtgtgcctggccacaggcttctaccccgac
cacgtggagctgagctggtgggtgaatgggaaggaggtgcacagtggggtcagcacagacccgcagcccctcaaggagcagccc
gccctcaatgactccagatactgcctgagcagccgcctgagggtctcggccaccttctggcagaacccccgcaaccacttccgctgt
caagtccagttctacgggctctcggagaatgacgagtggacccaggatagggccaaacctgtcacccagatcgtcagcgccgagg
cctggggtagagcagactgtggcttcacctccgagtcttaccagcaaggggtcctgtctgccaccatcctctatgagatcttgctagg
gaaggccaccttgtatgccgtgctggtcagtgccctcgtgctgatggccatggtcaagagaaaggattccagaggctag
SEQ ID NO: 35-11N6 TCRbeta-P2A-alpha Codon-optimized
ATGGGCACAAGACTTCTCTGTTGGGCTGCACTGTGCTTGCTTGGAGCTGAGCTGACAGAAGCTGGA
GTTGCCCAATCTCCTAGGTACAAGATCATCGAGAAGCGGCAGTCTGTGGCCTTTTGGTGCAATCCCA
TTAGCGGACATGCCACCCTGTACTGGTATCAGCAAATTCTGGGACAGGGCCCTAAACTGCTGATCCA
GTTCCAGAATAACGGCGTGGTGGACGATTCTCAACTGCCTAAGGACCGGTTTTCTGCCGAGAGACT
GAAAGGCGTTGATAGCACCCTGAAGATCCAACCTGCCAAACTGGAGGATTCTGCCGTGTACCTGTGT
GCTAGCAGCCCTTATGGAGGATCTGTGTCTTATGAGCAGTACTTCGGACCTGGCACCAGACTGACCG
TGACTGAAGACCTGAAGAACGTGTTCCCCCCAGAGGTGGCCGTGTTCGAGCCTAGCGAGGCCGAGA
TCAGCCACACCCAGAAAGCCACCCTCGTGTGCCTGGCCACCGGCTTTTACCCCGACCACGTGGAACT
GTCTTGGTGGGTCAACGGCAAAGAGGTGCACAGCGGCGTCTGCACCGACCCCCAGCCCCTGAAAGA
GCAGCCCGCCCTGAACGACAGCCGGTACTGTCTGAGCAGCAGACTGAGAGTGTCCGCCACCTTCTG
GCAGAACCCCCGGAACCACTTCAGATGCCAGGTGCAGTTCTACGGCCTGAGCGAGAACGACGAGTG
GACCCAGGACCGGGCCAAGCCCGTGACCCAGATCGTGTCTGCTGAGGCCTGGGGCAGAGCCGATT
GCGGCTTCACCAGCGAGAGCTACCAGCAGGGCGTGCTGAGCGCCACCATCCTGTACGAGATCCTGC
TGGGCAAGGCCACCCTGTACGCCGTGCTGGTGTCCGCCCTGGTGCTGATGGCCATGGTCAAGCGGA
AGGACAGCCGGGGCGGTTCCGGAGCCACGAACTTCTCTCTGTTAAAGCAAGCAGGAGACGTGGAA
GAAAACCCCGGTCCCATGGAGACACTGCTTGGCGTATCACTGGTGATTCTGTGGCTGCAACTGGCTA
GAGTGAACTCTCAGCAGGGAGAAGAGGATCCTCAAGCTCTGAGCATTCAGGAAGGCGAAAACGCA
ACCATGAATTGCTCATACAAGACCAGCATCAACAACCTGCAGTGGTACCGGCAGAATAGCGGAAGA
GGACTGGTTCACCTGATTTTAATCAGGTCTAATGAAAGGGAGAAGCACAGCGGCAGACTGAGAGTT
ACCCTGGACACATCCAAGAAATCTTCTTCTCTGCTGATTACAGCCTCTAGAGCCGCCGATACAGCCAG
CTACTTTTGTGCCACAGATCCCATGAACACCAATGCCGGAAAGAGCACATTCGGCGATGGCACAACC
CTGACAGTTAAGCCCAATATCCAGAATCCCGATCCTGCCGTGTACCAGCTGCGGGACAGCAAGAGC
AGCGACAAGAGCGTGTGCCTGTTCACCGACTTCGACAGCCAGACCAACGTGTCCCAGAGCAAGGAC
AGCGACGTGTACATCACCGATAAGTGCGTGCTGGACATGCGGAGCATGGACTTCAAGAGCAACAGC
GCCGTGGCCTGGTCCAACAAGAGCGACTTCGCCTGCGCCAACGCCTTCAACAACAGCATTATCCCCG
AGGACACATTCTTCCCAAGCCCCGAGAGCAGCTGCGACGTGAAGCTGGTGGAAAAGAGCTTCGAGA
CAGACACCAACCTGAACTTCCAGAACCTCAGCGTGATCGGCTTCCGGATCCTGCTGCTGAAGGTGGC
CGGCTTCAACCTGCTGATGACCCTGCGGCTGTGGTCCAGCTGA
SEQ ID NO: 36-CD8alpha-T2A-CD8beta-P2A-11N6 TCRbeta-P2A-alpha Codon-optimized
ATGGCTCTGCCTGTGACAGCTCTGCTGCTGCCTCTGGCTCTGCTTCTGCATGCCGCTAGACCCAGCCA
GTTCAGAGTGTCCCCTCTGGACAGAACCTGGAACCTGGGCGAGACAGTGGAACTGAAGTGCCAGGT
GCTGCTGAGCAATCCTACCAGCGGCTGCAGCTGGCTGTTTCAGCCTAGAGGTGCTGCCGCCTCTCCT
ACCTTTCTGCTGTACCTGAGCCAGAACAAGCCCAAGGCCGCCGAAGGACTGGACACCCAGAGATTC
AGCGGCAAGAGACTGGGCGACACCTTCGTGCTGACCCTGAGCGACTTCAGAAGAGAGAACGAGGG
CTACTACTTCTGCAGCGCCCTGAGCAACAGCATCATGTACTTCAGCCACTTCGTGCCCGTGTTTCTGC
CCGCCAAGCCTACAACAACCCCTGCTCCTAGACCTCCTACACCAGCTCCTACAATCGCCAGCCAGCCT
CTGTCTCTGAGGCCAGAAGCTTGTAGACCTGCTGCTGGCGGAGCCGTGCATACAAGAGGACTGGAT
TTCGCCTGCGACATCTACATCTGGGCCCCTCTGGCTGGAACATGTGGCGTGCTGCTGCTGTCCCTGG
TCATCACCCTGTACTGCAACCACCGGAACAGGCGGAGAGTGTGCAAGTGCCCTAGACCTGTGGTCA
AGAGCGGCGACAAGCCTAGCCTGAGCGCCAGATATGTTGGCAGCGGAGAAGGCAGAGGCTCCCTG
CTTACATGCGGCGACGTGGAAGAGAACCCCGGACCTATGAGGCCTAGACTGTGGCTGCTTCTGGCT
GCCCAGCTGACAGTGCTGCACGGCAATTCTGTCCTGCAGCAGACCCCTGCCTACATCAAGGTGCAGA
CCAACAAGATGGTCATGCTGAGCTGCGAGGCCAAGATCAGCCTGTCCAACATGCGGATCTACTGGC
TGCGGCAGAGACAGGCCCCTAGCTCTGATAGCCACCACGAGTTTCTGGCCCTGTGGGATTCTGCCAA
GGGCACCATTCACGGCGAGGAAGTGGAACAAGAGAAGATCGCCGTGTTCCGGGACGCCAGCAGAT
TCATCCTGAACCTGACCAGCGTGAAGCCCGAGGACAGCGGCATCTATTTCTGCATGATCGTGGGCA
GCCCCGAGCTGACATTTGGCAAGGGAACACAGCTGAGCGTGGTGGACTTCCTGCCTACTACAGCCC
AGCCTACCAAGAAGTCTACCCTGAAGAAACGCGTGTGCAGACTGCCCAGGCCTGAGACACAAAAGG
GCCCTCTGTGCAGCCCTATCACACTGGGATTGCTGGTGGCTGGCGTTCTGGTCCTGCTGGTGTCTCT
GGGAGTTGCCATCCACCTGTGCTGTAGAAGAAGGCGGGCCAGACTGCGGTTCATGAAGCAGTTCTA
CAAAGGCAGCGGCGCCACCAACTTCAGCCTGCTGAAACAAGCCGGCGACGTCGAGGAAAATCCTGG
ACCTATGGGCACAAGACTTCTCTGTTGGGCTGCACTGTGCTTGCTTGGAGCTGAGCTGACAGAAGCT
GGAGTTGCCCAATCTCCTAGGTACAAGATCATCGAGAAGCGGCAGTCTGTGGCCTTTTGGTGCAATC
CCATTAGCGGACATGCCACCCTGTACTGGTATCAGCAAATTCTGGGACAGGGCCCTAAACTGCTGAT
CCAGTTCCAGAATAACGGCGTGGTGGACGATTCTCAACTGCCTAAGGACCGGTTTTCTGCCGAGAG
ACTGAAAGGCGTTGATAGCACCCTGAAGATCCAACCTGCCAAACTGGAGGATTCTGCCGTGTACCTG
TGTGCTAGCAGCCCTTATGGAGGATCTGTGTCTTATGAGCAGTACTTCGGACCTGGCACCAGACTGA
CCGTGACTGAAGACCTGAAGAACGTGTTCCCCCCAGAGGTGGCCGTGTTCGAGCCTAGCGAGGCCG
AGATCAGCCACACCCAGAAAGCCACCCTCGTGTGCCTGGCCACCGGCTTTTACCCCGACCACGTGGA
ACTGTCTTGGTGGGTCAACGGCAAAGAGGTGCACAGCGGCGTCTGCACCGACCCCCAGCCCCTGAA
AGAGCAGCCCGCCCTGAACGACAGCCGGTACTGTCTGAGCAGCAGACTGAGAGTGTCCGCCACCTT
CTGGCAGAACCCCCGGAACCACTTCAGATGCCAGGTGCAGTTCTACGGCCTGAGCGAGAACGACGA
GTGGACCCAGGACCGGGCCAAGCCCGTGACCCAGATCGTGTCTGCTGAGGCCTGGGGCAGAGCCG
ATTGCGGCTTCACCAGCGAGAGCTACCAGCAGGGCGTGCTGAGCGCCACCATCCTGTACGAGATCC
TGCTGGGCAAGGCCACCCTGTACGCCGTGCTGGTGTCCGCCCTGGTGCTGATGGCCATGGTCAAGC
GGAAGGACAGCCGGGGCGGTTCCGGAGCCACCAACTTCAGCCTGCTTAAACAGGCCGGCGACGTG
GAAGAGAACCCTGGACCTATGGAGACACTGCTTGGCGTATCACTGGTGATTCTGTGGCTGCAACTG
GCTAGAGTGAACTCTCAGCAGGGAGAAGAGGATCCTCAAGCTCTGAGCATTCAGGAAGGCGAAAA
CGCAACCATGAATTGCTCATACAAGACCAGCATCAACAACCTGCAGTGGTACCGGCAGAATAGCGG
AAGAGGACTGGTTCACCTGATTTTAATCAGGTCTAATGAAAGGGAGAAGCACAGCGGCAGACTGAG
AGTTACCCTGGACACATCCAAGAAATCTTCTTCTCTGCTGATTACAGCCTCTAGAGCCGCCGATACAG
CCAGCTACTTTTGTGCCACAGATCCCATGAACACCAATGCCGGAAAGAGCACATTCGGCGATGGCAC
AACCCTGACAGTTAAGCCCAATATCCAGAATCCCGATCCTGCCGTGTACCAGCTGCGGGACAGCAAG
AGCAGCGACAAGAGCGTGTGCCTGTTCACCGACTTCGACAGCCAGACCAACGTGTCCCAGAGCAAG
GACAGCGACGTGTACATCACCGATAAGTGCGTGCTGGACATGCGGAGCATGGACTTCAAGAGCAAC
AGCGCCGTGGCCTGGTCCAACAAGAGCGACTTCGCCTGCGCCAACGCCTTCAACAACAGCATTATCC
CCGAGGACACATTCTTCCCAAGCCCCGAGAGCAGCTGCGACGTGAAGCTGGTGGAAAAGAGCTTCG
AGACAGACACCAACCTGAACTTCCAGAACCTCAGCGTGATCGGCTTCCGGATCCTGCTGCTGAAGGT
GGCCGGCTTCAACCTGCTGATGACCCTGCGGCTGTGGTCCAGCTGA
SEQ ID NO: 37-11N6 TCR alpha chain-original protein, with signal peptide underlined
METLLGVSLVILWLQLARVNSQQGEEDPQALSIQEGENATMNCSYKTSINNLQWYRQNSGRGLVHLILI
RSNEREKHSGRLRVTLDTSKKSSSLLITASRAADTASYFCATDPMNTNAGKSTFGDGTTLTVKPNIQNPDP
AVYQLRDSKSSDKSVCLFTDFDSQTNVSQSKDSDVYITDKTVLDMRSMDFKSNSAVAWSNKSDFACAN
AFNNSIIPEDTFFPSPESSCDVKLVEKSFETDTNLNFQNLSVIGFRILLLKVAGFNLLMTLRLWSS*
SEQ ID NO: 38-11N6 TCR alpha chain-original protein, without signal peptide
QQGEEDPQALSIQEGENATMNCSYKTSINNLQWYRQNSGRGLVHLILIRSNEREKHSGRLRVTLDTSKKS
SSLLITASRAADTASYFCATDPMNTNAGKSTFGDGTTLTVKPNIQNPDPAVYQLRDSKSSDKSVCLFTDFD
SQTNVSQSKDSDVYITDKTVLDMRSMDFKSNSAVAWSNKSDFACANAFNNSIIPEDTFFPSPESSCDVKL
VEKSFETDTNLNFQNLSVIGFRILLLKVAGFNLLMTLRLWSS*
SEQ ID NO: 39-11N6 TCR alpha chain variable domain, without signal peptide
QQGEEDPQALSIQEGENATMNCSYKTSINNLQWYRQNSGRGLVHLILIRSNEREKHSGRLRVTLDTSKKS
SSLLITASRAADTASYFCATDPMNTNAGKSTFGDGTTLTVKP
SEQ ID NO: 40--11N6 TCR alpha chain variable domain CDR1α
TSINN
SEQ ID NO: 41-11N6 TCR alpha chain variable domain CDR2α
IRSNERE
SEQ ID NO: 42-11N6 TCR alpha chain variable domain CDR3α-IMGT junction
CATDPMNTNAGKSTF
SEQ ID NO: 43-11N6 TCR alpha chain variable domain CDR3α-IMGT
ATDPMNTNAGKST
SEQ ID NO: 44-11N6 TCR alpha chain constant domain (original protein)
NIQNPDPAVYQLRDSKSSDKSVCLFTDFDSQTNVSQSKDSDVYITDKTVLDMRSMDFKSNSAVAWSNK
SDFACANAFNNSIIPEDTFFPSPESSCDVKLVEKSFETDTNLNFQNLSVIGFRILLLKVAGFNLLMTLRLWSS
SEQ ID NO: 45-11N6 TCR alpha chain constant domain (cys-modified protein)
NIQNPDPAVYQLRDSKSSDKSVCLFTDFDSQTNVSQSKDSDVYITDKCVLDMRSMDFKSNSAVAWSNK
SDFACANAFNNSIIPEDTFFPSPESSCDVKLVEKSFETDTNLNFQNLSVIGFRILLLKVAGFNLLMTLRLWSS
SEQ ID NO: 46-11N6 TCR alpha chain, without signal peptide, cys-modified
QQGEEDPQALSIQEGENATMNCSYKTSINNLQWYRQNSGRGLVHLILIRSNEREKHSGRLRVTLDTSKKS
SSLLITASRAADTASYFCATDPMNTNAGKSTFGDGTTLTVKPNIQNPDPAVYQLRDSKSSDKSVCLFTDFD
SQTNVSQSKDSDVYITDKCVLDMRSMDFKSNSAVAWSNKSDFACANAFNNSIIPEDTFFPSPESSCDVKL
VEKSFETDTNLNFQNLSVIGFRILLLKVAGFNLLMTLRLWSS
SEQ ID NO: 47-11N6 TCR beta chain original protein, with signal peptide underlined
MGTRLLCWAALCLLGAELTEAGVAQSPRYKIIEKRQSVAFWCNPISGHATLYWYQQILGQGPKLLIQFQN
NGVVDDSQLPKDRFSAERLKGVDSTLKIQPAKLEDSAVYLCASSPYGGSVSYEQYFGPGTRLTVTEDLKNV
FPPEVAVFEPSEAEISHTQKATLVCLATGFYPDHVELSWWVNGKEVHSGVSTDPQPLKEQPALNDSRYCL
SSRLRVSATFWQNPRNHFRCQVQFYGLSENDEWTQDRAKPVTQIVSAEAWGRADCGFTSESYQQGVL
SATILYEILLGKATLYAVLVSALVLMAMVKRKDSRG
SEQ ID NO: 48-11N6 TCR beta chain original protein, without signal peptide
GVAQSPRYKIIEKRQSVAFWCNPISGHATLYWYQQILGQGPKLLIQFQNNGVVDDSQLPKDRFSAERLK
GVDSTLKIQPAKLEDSAVYLCASSPYGGSVSYEQYFGPGTRLTVTEDLKNVFPPEVAVFEPSEAEISHTQKA
TLVCLATGFYPDHVELSWWVNGKEVHSGVSTDPQPLKEQPALNDSRYCLSSRLRVSATFWQNPRNHFR
CQVQFYGLSENDEWTQDRAKPVTQIVSAEAWGRADCGFTSESYQQGVLSATILYEILLGKATLYAVLVSA
LVLMAMVKRKDSRG
SEQ ID NO: 49-11N6 TCR beta chain variable domain, without signal peptide
GVAQSPRYKIIEKRQSVAFWCNPISGHATLYWYQQILGQGPKLLIQFQNNGVVDDSQLPKDRFSAERLK
GVDSTLKIQPAKLEDSAVYLCASSPYGGSVSYEQYFGPGTRLTVT
SEQ ID NO: 50-11N6 TCR beta chain variable domain CDR1ß
SGHAT
SEQ ID NO: 51-11N6 TCR beta chain variable domain CDR2ß
FQNNGV
SEQ ID NO: 52-11N6 TCR beta chain variable domain CDR3ß-IMGT junction
CASSPYGGSVSYEQYF
SEQ ID NO: 53-11N6 TCR beta chain variable domain CDR3ß-IMGT
ASSPYGGSVSYEQY
SEQ ID NO: 54 11N6 TCR beta chain constant domain (original protein)
EDLKNVFPPEVAVFEPSEAEISHTQKATLVCLATGFYPDHVELSWWVNGKEVHSGVSTDPQPLKEQPAL
NDSRYCLSSRLRVSATFWQNPRNHFRCQVQFYGLSENDEWTQDRAKPVTQIVSAEAWGRADCGFTSE
SYQQGVLSATILYEILLGKATLYAVLVSALVLMAMVKRKDSRG
SEQ ID NO: 55-11N6 TCR beta chain constant domain (cys-modified protein)
EDLKNVFPPEVAVFEPSEAEISHTQKATLVCLATGFYPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCGFTSE
SYQQGVLSATILYEILLGKATLYAVLVSALVLMAMVKRKDSRG
SEQ ID NO: 56-11N6 TCR beta chain (cys-modified protein)
GVAQSPRYKIIEKRQSVAFWCNPISGHATLYWYQQILGQGPKLLIQFQNNGVVDDSQLPKDRFSAERLK
GVDSTLKIQPAKLEDSAVYLCASSPYGGSVSYEQYFGPGTRLTVTEDLKNVFPPEVAVFEPSEAEISHTQKA
TLVCLATGFYPDHVELSWWVNGKEVHSGVCTDPQPLKEQPALNDSRYCLSSRLRVSATFWQNPRNHFR
CQVQFYGLSENDEWTQDRAKPVTQIVSAEAWGRADCGFTSESYQQGVLSATILYEILLGKATLYAVLVSA
LVLMAMVKRKDSRG
SEQ ID NO: 57-11N6 TCRbeta-P2A-alpha-Protein, with signal peptides underlined
MGTRLLCWAALCLLGAELTEAGVAQSPRYKIIEKRQSVAFWCNPISGHATLYWYQQILGQGPKLLIQFQN
NGVVDDSQLPKDRFSAERLKGVDSTLKIQPAKLEDSAVYLCASSPYGGSVSYEQYFGPGTRLTVTEDLKNV
FPPEVAVFEPSEAEISHTQKATLVCLATGFYPDHVELSWWVNGKEVHSGVCTDPQPLKEQPALNDSRYC
LSSRLRVSATFWQNPRNHFRCQVQFYGLSENDEWTQDRAKPVTQIVSAEAWGRADCGFTSESYQQGV
LSATILYEILLGKATLYAVLVSALVLMAMVKRKDSRGGSGATNFSLLKQAGDVEENPGPMETLLGVSLVIL
WLQLARVNSQQGEEDPQALSIQEGENATMNCSYKTSINNLQWYRQNSGRGLVHLILIRSNEREKHSGRL
RVTLDTSKKSSSLLITASRAADTASYFCATDPMNTNAGKSTFGDGTTLTVKPNIQNPDPAVYQLRDSKSSD
KSVCLFTDFDSQTNVSQSKDSDVYITDKCVLDMRSMDFKSNSAVAWSNKSDFACANAFNNSIIPEDTFF
PSPESSCDVKLVEKSFETDTNLNFQNLSVIGFRILLLKVAGFNLLMTLRLWSS*
SEQ ID NO: 58-CD8alpha-T2A-CD8beta-P2A-11N6 TCRbeta-P2A-alpha-Protein,
with signal peptides underlined
MALPVTALLLPLALLLHAARPSQFRVSPLDRTWNLGETVELKCQVLLSNPTSGCSWLFQPRGAAASPTFL
LYLSQNKPKAAEGLDTQRFSGKRLGDTFVLTLSDFRRENEGYYFCSALSNSIMYFSHFVPVFLPAKPTTTPA
PRPPTPAPTIASQPLSLRPEACRPAAGGAVHTRGLDFACDIYIWAPLAGTCGVLLLSLVITLYCNHRNRRR
VCKCPRPVVKSGDKPSLSARYVGSGEGRGSLLTCGDVEENPGPMRPRLWLLLAAQLTVLHGNSVLQQTP
AYIKVQTNKMVMLSCEAKISLSNMRIYWLRQRQAPSSDSHHEFLALWDSAKGTIHGEEVEQEKIAVFRD
ASRFILNLTSVKPEDSGIYFCMIVGSPELTFGKGTQLSVVDFLPTTAQPTKKSTLKKRVCRLPRPETQKGPLC
SPITLGLLVAGVLVLLVSLGVAIHLCCRRRRARLRFMKQFYKGSGATNFSLLKQAGDVEENPGPMGTRLLC
WAALCLLGAELTEAGVAQSPRYKIIEKRQSVAFWCNPISGHATLYWYQQILGQGPKLLIQFQNNGVVDD
SQLPKDRFSAERLKGVDSTLKIQPAKLEDSAVYLCASSPYGGSVSYEQYFGPGTRLTVTEDLKNVFPPEVAV
FEPSEAEISHTQKATLVCLATGFYPDHVELSWWVNGKEVHSGVCTDPQPLKEQPALNDSRYCLSSRLRVS
ATFWQNPRNHFRCQVQFYGLSENDEWTQDRAKPVTQIVSAEAWGRADCGFTSESYQQGVLSATILYEI
LLGKATLYAVLVSALVLMAMVKRKDSRGGSGATNFSLLKQAGDVEENPGPMETLLGVSLVILWLQLARV
NSQQGEEDPQALSIQEGENATMNCSYKTSINNLQWYRQNSGRGLVHLILIRSNEREKHSGRLRVTLDTSK
KSSSLLITASRAADTASYFCATDPMNTNAGKSTFGDGTTLTVKPNIQNPDPAVYQLRDSKSSDKSVCLFTD
FDSQTNVSQSKDSDVYITDKCVLDMRSMDFKSNSAVAWSNKSDFACANAFNNSIIPEDTFFPSPESSCD
VKLVEKSFETDTNLNFQNLSVIGFRILLLKVAGFNLLMTLRLWSS*
SEQ ID NO: 59-TCR BNT VB, with signal peptide
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LADIYEQYFGPGTRLTVT
SEQ ID NO: 60-TCR BNT Va, with signal peptide
METLLGVSLVILWLQLARVNSQQGEEDPQALSIQEGENATMNCSYKTSINNLQWYR
QNSGRGLVHLILIRSNEREKHSGRLRVTLDTSKKSSSLLITASRAADTASYFCATDRQ
SSGDKLTFGTGTRLAVRP
SEQ ID NO: 61-(TCR 220_21 Vα)
GEDVEQSLFLSVREGDSSVINCTYTDSSSTYLYWYKQEPGAGLQLLTYIFSNMDMKQ
DQRLTVLLNKKDKHLSLRIADTQTGDSAIYFCAEPIIGGNTPLVFGKGTRLSVIAN
SEQ ID NO: 62 (TCR 220_21 Vß)
GAGVSQSPRYKVAKRGQDVALRCDPISGHVSLFWYQQALGQGPEFLTYFQNEAQLD
KSGLPSDRFFAERPEGSVSTLKIQRTQQEDSAVYLCASSSEGLAGGPTAGELFFGEGS
RLTVL
SEQ ID NO: 63 (TCR 129_5 Vα)
AQSVTQPDIHITVSEGASLELRCNYSYGATPYLFWYVQSPGQGLQLLLKYFSGDTLV
QGIKGFEAEFKRSQSSFNLRKPSVHWSDAAEYFCAVGASGTYKYIFGTGTRLKVLAN
SEQ ID NO: 64 (TCR 129_5 Vß)
DAGVIQSPRHEVTEMGQEVTLRCKPISGHNSLFWYRQTMMRGLELLIYFNNNVPIDD
SGMPEDRFSAKMPNASFSTLKIQPSEPRDSAVYFCASSLALSYEQYFGPGTRLTVT
SEQ ID NO: 65-CD8α (Homo Sapiens, UniProt accession P01732, signal peptide underlined)
MALPVTALLLPLALLLHAARPSQFRVSPLDRTWNLGETVELKCQVLLSNPTSGCSWL
FQPRGAAASPTFLLYLSQNKPKAAEGLDTQRFSGKRLGDTFVLTLSDFRRENEGYYF
CSALSNSIMYFSHFVPVFLPAKPTTTPAPRPPTPAPTIASQPLSLRPEACRPAAGGAVH
TRGLDFACDIYIWAPLAGTCGVLLLSLVITLYCNHRNRRRVCKCPRPVVKSGDKPSL
SARYV
SEQ ID NO: 66-CD8ß (Homo Sapiens, UniProt accession P10966, signal peptide underlined)
MRPRLWLLLAAQLTVLHGNSVLQQTPAYIKVQTNKMVMLSCEAKISLSNMRIYWLR
QRQAPSSDSHHEFLALWDSAKGTIHGEEVEQEKIAVFRDASRFILNLTSVKPEDSGIY
FCMIVGSPELTFGKGTQLSVVDFLPTTAQPTKKSTLKKRVCRLPRPETQKGPLCSPITL
GLLVAGVLVLLVSLGVAIHLCCRRRRARLRFMKQFYK
SEQ ID NO: 67-[reserved]
SEQ ID NO: 68-[reserved]
SEQ ID NO: 69-TCR Ca amino acid sequence engineered to include threonine-to-
cysteine and LVL mutations
NIQNPDPAVYQLRDSKSSDKSVCLFTDFDSQTNVSQSKDSDVYITDKCVLDMRSMDF
KSNSAVAWSNKSDFACANAFNNSIIPEDTFFPSPESSCDVKLVEKSFETDTNLNFQNL
LVIVLRILLLKVAGFNLLMTLRLWSS
SEQ ID NO: 70-TRBC1 amino acid sequence (UniProt KB P01850)
EDLNKVFPPEV AVFEPSEAEI SHTQKATLVC LATGFFPDHV ELSWWVNGKE
VHSGVSTDPQ PLKEQPALND SRYCLSSRLR VSATFWQNPR NHFRCQVQFY
GLSENDEWTQ DRAKPVTQIV SAEAWGRADC GFTSVSYQQG VLSATILYEI
LLGKATLYAV LVSALVLMAM VKRKDF
SEQ ID NO: 71-TRBC1 amino acid sequence (cys-modified)
EDLNKVFPPEVAVFEPSEAEISHTQKATLVCLATGFFPDHVELSWWVNGKEVHSGVC
TDPQPLKEQPALNDSRYCLSSRLRVSATFWQNPRNHFRCQVQFYGLSENDEWTQDR
AKPVTQIVSAEAWGRADCGFTSVSYQQGVLSATILYEILLGKATLYAVLVSALVLMA
MVKRKDF
SEQ ID NO: 72-TRBC2 amino acid sequence (UniProt KB A0A5B9)
EDLKNVFPPKV AVFEPSEAEI SHTQKATLVC LATGFYPDHV ELSWWVNGKE
VHSGVSTDPQ PLKEQPALND SRYCLSSRLR VSATFWQNPR NHFRCQVQFY
GLSENDEWTQ DRAKPVTQIV SAEAWGRADC GFTSESYQQG VLSATILYEI
LLGKATLYAV LVSALVLMAM VKRKDSRG
SEQ ID NO: 73-TRBC2 amino acid sequence (cys-modified)
EDLKNVFPPKV AVFEPSEAEI SHTQKATLVC LATGFYPDHV ELSWWVNGKE
VHSGVCTDPQ PLKEQPALND SRYCLSSRLR VSATFWQNPR NHFRCQVQFY
GLSENDEWTQ DRAKPVTQIV SAEAWGRADC GFTSESYQQG VLSATILYEI
LLGKATLYAV LVSALVLMAM VKRKDSRG
(SEQ ID NO: 74) Porcine teschovirus-1 2A (P2A) self-cleaving peptide with N-terminal
GSG linker
GSGATNFSLLKQAGDVEENPGP
(SEQ ID NO: 75) Thoseaasigna virus 2A (T2A) self-cleaving peptide
LEGGGEGRGSLLTCGDVEENPGPR
(SEQ ID NO: 76) Equine rhinitis A virus (ERAV) 2A (E2A) self-cleaving peptide
QCTNYALLKLAGDVESNPGP
(SEQ ID NO: 77) Foot-and-Mouth disease virus 2A (F2A) self-cleaving peptide with N-
terminal G-S-G linker
GSGVKQTLNFDLLKLAGDVESNPGP
(SEQ ID NO: 78) NRAS (Uniprot KB P01111)
MTEYKLVVVG AGGVGKSALT IQLIQNHFVD EYDPTIEDSY RKQVVIDGET
CLLDILDTAG QEEYSAMRDQ YMRTGEGFLC VFAINNSKSF ADINLYREQI
KRVKDSDDVP MVLVGNKCDL PTRTVDTKQA HELAKSYGIP FIETSAKTRQ
GVEDAFYTLV REIRQYRMKK LNSSDDGTQG CMGLPCVVM
(SEQ ID NO: 79) HRAS (Uniprot KB P01112)
MTEYKLVVVG AGGVGKSALT IQLIQNHFVD EYDPTIEDSY RKQVVIDGET
CLLDILDTAG QEEYSAMRDQ YMRTGEGFLC VFAINNTKSF EDIHQYREQI
KRVKDSDDVP MVLVGNKCDL AARTVESRQA QDLARSYGIP YIETSAKTRQ
GVEDAFYTLV REIRQHKLRK LNPPDESGPG CMSCKCVLS
(SEQ ID NO: 80) Fas-41BB Fusion (amino acid)
MLGIWTLLPLVLTSVARLSSKSVNAQVTDINSKGLELRKTVTTVETQNLEGLHHDGQ
FCHKPCPPGERKARDCTVNGDEPDCVPCQEGKEYTDKAHFSSKCRRCRLCDEGHGL
EVEINCTRTQNTKCRCKPNFFCNSTVCEHCDPCTKCEHGIIKECTLTSNTKCKEEGSR
SNLGWLCLLLLPIPLIVWVKRGRKKLLYIFKQPFMRPVQTTQEEDGCSCRFPEEEEGG
CEL
(SEQ ID NO: 81) FAS extracellular domain containing fragment
MLGIWTLLPLVLTSVARLSSKSVNAQVTDINSKGLELRKTVTTVETQNLEGLHHDGQ
FCHKPCPPGERKARDCTVNGDEPDCVPCQEGKEYTDKAHFSSKCRRCRLCDEGHGL
EVEINCTRTQNTKCRCKPNFFCNSTVCEHCDPCTKCEHGIIKECTLTSNTKCKEEGSR
SN
(SEQ ID NO: 82) 41BB Intracellular domain-containing fragment
LCLLLLPIPLIVWVKRGRKKLLYIFKQPFMRPVQTTQEEDGCCRFPEEEEGGCEL
(SEQ ID NO: 83) FAS-41BB Fusion (Nucleotide encoding sequence)
ATGCTGGGCATCTGGACCCTGCTGCCCTTGGTGCTGACTAGCGTGGCTAGACTGA
GCAGCAAGAGCGTGAACGCCCAAGTGACCGACATCAACAGCAAGGGCCTGGAG
CTGAGAAAGACCGTGACCACCGTGGAGACACAGAACCTGGAGGGCCTGCACCAC
GACGGGCAGTTCTGCCACAAGCCCTGCCCCCCCGGCGAGAGAAAGGCTAGAGAC
TGCACCGTGAACGGCGACGAGCCCGACTGCGTGCCCTGCCAAGAGGGCAAGGAG
TACACCGACAAGGCCCACTTCAGCAGCAAGTGCAGAAGATGCAGACTGTGCGAC
GAGGGCCACGGCCTGGAGGTGGAGATCAACTGCACGCGTACGCAGAATACCAAA
TGCCGCTGCAAGCCCAACTTCTTCTGCAACAGCACCGTGTGCGAGCACTGCGACC
CCTGCACCAAGTGCGAGCACGGCATCATCAAGGAGTGCACCCTGACAAGCAACA
CCAAGTGTAAGGAAGAGGGCTCACGGAGCAACCTGGGCTGGCTGTGCCTGCTGC
TGCTGCCCATCCCCCTGATCGTGTGGGTGAAGAGAGGCAGAAAGAAGCTGCTGT
ACATCTTCAAGCAGCCCTTCATGAGACCCGTGCAGACCACCCAAGAGGAGGACG
GGTGCAGCTGTAGATTCCCCGAAGAAGAAGAAGGCGGCTGTGAGCTT

TABLE 2
Additional Sequences of Polypeptide and Nucleic acid Components described
herein

| SEQ | | |
|---|---|---|
| ID | | |
| NO | Target | Sequence (Amino acid or DNA) |
| 84 | KRAS | LVVVGAAGV |
| 85 | KRAS | LLVVGAAGV |
| 86 | KRAS | LMVVGAAGV |
| 87 | KRAS | LVVVGACGV |
| 88 | KRAS | LLVVGACGV |
| 89 | KRAS | LMVVGACGV |
| 90 | KRAS | LVVVGAVGV |
| 91 | KRAS | LLVVGAVGV |
| 92 | KRAS | LMVVGAVGV |
| 93 | PIK3CA | GLKDLLNPI |
| 94 | PIK3CA | GLKDLLNPV |
| 95 | PIK3CA | GMKDLLNPV |
| 96 | PIK3CA | ILNREIDFA |
| 97 | PIK3CA | ILNREIDFV |
| 98 | PIK3CA | IMNREIDFV |
| 99 | PIK3CA | ILNREIDFL |
| 100 | PIK3CA | HAGLSNRLARDNELRENDKEQLKAISTRDPLSEITEQEKDFLWSH |
| | | RHYCVTIPEILPKLLLSVKW |
| 101 | PIK3CA | HAGLSNRLARDNELRENDKEQLKAI |
| 102 | PIK3CA | LSNRLARDNELRENDKEQLKAISTRDPLSEITEQEKDFLWSHRHY |
| | | CVTIPEILPKLLLSVKWNSR |
| 103 | PIK3CA | LSNRLARDNELRENDKEQLKAISTRDPLSEITKQEKDFLWSHRHY |
| | | CVTIPEILPKLLLSVKWNSR |
| 104 | PIK3CA | KTLALDKTEQEALEYFMKQMNDAHHGGWTTKMDWIFHTIKQHA |
| 105 | PIK3CA | KTLALDKTEQEALEYFMKQMNDARHGGWTTKMDWIFHTIKQHA |
| 106 | PIK3CA | KTLALDKTEQEALEYFMKQMNDALHGGWTTKMDWIFHTIKQHA |
| 107 | PIK3CA | DSAIYN |
| 108 | PIK3CA | IQSSQRE |
| 109 | PIK3CA | CAVKGSDDYKLSF |
| 110 | PIK3CA | KGHSH |
| 111 | PIK3CA | KGHSH |
| 112 | PIK3CA | CASSPVNLAGVSRADTQYF |
| 113 | PIK3CA | METLLGLLILWLQLQWVSSKQEVTQIPAALSVPEGENLVLNCSFT |
| | | DSAIYNLQWFRQDPGKGLTSLLLIQSSQREQTSGRLNASLDKSSGR |
| | | STLYIAASQPGDSATYLCAVKGSDDYKLSFGAGTTVTVRA |
| 114 | PIK3CA | METLLGLLILWLQLQWVSSKQEVTQIPAALSVPEGENLVLNCSFT |
| | | DSAIYNLQWFRQDPGKGLTSLLLIQSSQREQTSGRLNASLDKSSGR |
| | | STLYIAASQPGDSATYLCAVKGSDDYKLSFGAGTTVTVRANIQNP |
| | | EPAVYQLKDPRSQDSTLCLFTDFDSQINVPKTMESGTFITDKCVLD |
| | | MKAMDSKSNGAIAWSNQTSFTCQDIFKETNATYPSSDVPCDATLT |
| | | EKSFETDMNLNFQNLLVIVLRILLLKVAGFNLLMTLRLWSS |
| 115 | PIK3CA | MDTRVLCCAVICLLGAGLSNAGVMQNPRHLVRRRGQEARLRCSP |
| | | MKGHSHVYWYRQLPEEGLKFMVYLQKENIIDESGMPKERFSAEF |
| | | PKEGPSILRIQQVVRGDSAAYFCASSPVNLAGVSRADTQYFGPGT |
| | | RLTVL |
| 116 | PIK3CA | MDTRVLCCAVICLLGAGLSNAGVMQNPRHLVRRRGQEARLRCSP |
| | | MKGHSHVYWYRQLPEEGLKFMVYLQKENIIDESGMPKERFSAEF |
| | | PKEGPSILRIQQVVRGDSAAYFCASSPVNLAGVSRADTQYFGPGT |
| | | RLTVLEDLRNVTPPKVSLFEPSKAEIANKQKATLVCLARGFFPDH |
| | | VELSWWVNGKEVHSGVCTDPQAYKESNYSYCLSSRLRVSATFW |
| | | HNPRNHFRCQVQFHGLSEEDKWPEGSPKPVTQNISAEAWGRADC |
| | | GITSASYQQGVLSATILYEILLGKATLYAVLVSTLVVMAMVKRKN |
| | | S |
| 117 | PIK3CA | TSGFNG |
| 118 | PIK3CA | NVLDGL |
| 119 | PIK3CA | CAVTSWGKLQF |
| 120 | PIK3CA | SGHTA |
| 121 | PIK3CA | FQGNSA |
| 122 | PIK3CA | CASSPRGYQPQHF |
| 123 | PIK3CA | MWGVFLLYVSMKMGGTTGQNIDQPTEMTATEGAIVQINCTYQTS |
| | | GFNGLFWYQQHAGEAPTFLSYNVLDGLEEKGRFSSFLSRSKGYSY |
| | | LLLKELQMKDSASYLCAVTSWGKLQFKLQFGAGTQVVVTP |
| 124 | PIK3CA | MWGVFLLYVSMKMGGTTGQNIDQPTEMTATEGAIVQINCTYQTS |
| | | GFNGLFWYQQHAGEAPTFLSYNVLDGLEEKGRFSSFLSRSKGYSY |
| | | LLLKELQMKDSASYLCAVTSWGKLQFKLQFGAGTQVVVTPNIQN |
| | | PEPAVYQLKDPRSQDSTLCLFTDFDSQINVPKTMESGTFITDKCVL |
| | | DMKAMDSKSNGAIAWSNQTSFTCQDIFKETNATYPSSDVPCDATL |
| | | TEKSFETDMNLNFQNLLVIVLRILLLKVAGFNLLMTLRLWSS |
| 125 | PIK3CA | MGTRLLFWVAFCLLGAYHTGAGVSQSPSNKVTEKGKDVELRCDP |
| | | ISGHTALYWYRQRLGQGLEFLIYFQGNSAPDKSGLPSDRFSAERT |
| | | GESVSTLTIQRTQQEDSAVYLCASSPRGYQPQHFGDGTRLSIL |
| 126 | PIK3CA | MGTRLLFWVAFCLLGAYHTGAGVSQSPSNKVTEKGKDVELRCDP |
| | | ISGHTALYWYRQRLGQGLEFLIYFQGNSAPDKSGLPSDRFSAERT |
| | | GESVSTLTIQRTQQEDSAVYLCASSPRGYQPQHFGDGTRLSILEDL |
| | | RNVTPPKVSLFEPSKAEIANKQKATLVCLARGFFPDHVELSWWVN |
| | | GKEVHSGVCTDPQAYKESNYSYCLSSRLRVSATFWHNPRNHFRC |
| | | QVQFHGLSEEDKWPEGSPKPVTQNISAEAWGRADCGITSASYQQ |
| | | GVLSATILYEILLGKATLYAVLVSTLVVMAMVKRKNS |
| 127 | PIK3CA | TSDQSYG |
| 128 | PIK3CA | QGSYDEQN |
| 129 | PIK3CA | CAMREVLDNTDKLIF |
| 130 | PIK3CA | KGHSH |
| 131 | PIK3CA | LQKENI |
| 132 | PIK3CA | CASSPPEAGLDTEAFF |
| 133 | PIK3CA | MSLSSLLKVVTASLWLGPGIAQKITQTQPGMFVQEKEAVTLDCTY |
| | | DTSDQSYGLFWYKQPSSGEMIFLIYQGSYDEQNATEGRYSLNFQK |
| | | ARKSANLVISASQLGDSAMYFCAMREVLDNTDKLIFGTGTRLQVF |
| | | P |
| 134 | PIK3CA | MSLSSLLKVVTASLWLGPGIAQKITQTQPGMFVQEKEAVTLDCTY |
| | | DTSDQSYGLFWYKQPSSGEMIFLIYQGSYDEQNATEGRYSLNFQK |
| | | ARKSANLVISASQLGDSAMYFCAMREVLDNTDKLIFGTGTRLQVF |
| | | PNIQNPEPAVYQLKDPRSQDSTLCLFTDFDSQINVPKTMESGTFITD |
| | | KCVLDMKAMDSKSNGAIAWSNQTSFTCQDIFKETNATYPSSDVP |
| | | CDATLTEKSFETDMNLNFQNLLVIVLRILLLKVAGFNLLMTLRLW |
| | | SS |
| 135 | PIK3CA | MDTRVLCCAVICLLGAGLSNAGVMQNPRHLVRRRGQEARLRCSP |
| | | MKGHSHVYWYRQLPEEGLKFMVYLQKENIIDESGMPKERFSAEF |
| | | PKEGPSILRIQQVVRGDSAAYFCASSPPEAGLDTEAFFGQGTRLTV |
| | | V |
| 136 | PIK3CA | MDTRVLCCAVICLLGAGLSNAGVMQNPRHLVRRRGQEARLRCSP |
| | | MKGHSHVYWYRQLPEEGLKFMVYLQKENIIDESGMPKERFSAEF |
| | | PKEGPSILRIQQVVRGDSAAYFCASSPPEAGLDTEAFFGQGTRLTV |
| | | VEDLRNVTPPKVSLFEPSKAEIANKQKATLVCLARGFFPDHVELS |
| | | WWVNGKEVHSGVCTDPQAYKESNYSYCLSSRLRVSATFWHNPR |
| | | NHFRCQVQFHGLSEEDKWPEGSPKPVTQNISAEAWGRADCGITSA |
| | | SYQQGVLSATILYEILLGKATLYAVLVSTLVVMAMVKRKNS |
| 137 | PIK3CA | NSAFQY |
| 138 | PIK3CA | TYSSGN |
| 139 | PIK3CA | CAMNSGGYQKVTF |
| 140 | PIK3CA | DFQATT |
| 141 | PIK3CA | SNEGSKA |
| 142 | PIK3CA | CSAREQGPLEEQYF |
| 143 | PIK3CA | MMKSLRVLLVILWLQLSWVWSQQKEVEQDPGPLSVPEGAIVSLN |
| | | CTYSNSAFQYFMWYRQYSRKGPELLMYTYSSGNKEDGRFTAQV |
| | | DKSSKYISLFIRDSQPSDSATYLCAMNSGGYQKVTFGIGTKLQVIP |
| 144 | PIK3CA | MMKSLRVLLVILWLQLSWVWSQQKEVEQDPGPLSVPEGAIVSLN |
| | | CTYSNSAFQYFMWYRQYSRKGPELLMYTYSSGNKEDGRFTAQV |
| | | DKSSKYISLFIRDSQPSDSATYLCAMNSGGYQKVTFIFTKLQVIPNI |
| | | QNPEPAVYQLKDPRSQDSTLCLFTDFDSQINVPKTMESGTFITDKC |
| | | VLDMKAMDSKSNGAIAWSNQTSFTCQDIFKETNATYPSSDVPCD |
| | | ATLTEKSFETDMNNFQNLLVIVLRILLLKVAGFNLLMTLRLWSS |
| 145 | PIK3CA | MLLLLLLLGPAGSGLGAVVSQHPSRVICKSGTSVKIECRSLDFQAT |
| | | TMFWYRQFPKQSLMLMATSNEGSKATYEQGVEKDKFLINHASLT |
| | | LSTLTVTSAHPEDSSFYICSACSAREQGPLEEQYFGPGTRLTVT |
| 146 | PIK3CA | MLLLLLLLGPAGSGLGAVVSQHPSRVICKSGTSVKIECRSLDFQAT |
| | | TMFWYRQFPKQSLMLMATSNEGSKATYEQGVEKDKFLINHASLT |
| | | LSTLTVTSAHPEDSSFYICSACSAREQGPLEEQYFGPGTRLTVTEDL |
| | | RNVTPPKVSLFEPSKAEIANKQKATLVCLARGFFPDHVELSWWVN |
| | | GKEVHSGVCTDPQAYKESNYSYCLSSRLRVSATFWHNPRNHFRC |
| | | QVQFHGLSEEDKWPEGSPKPVTQNISAEAWGRADCGITSASYQQ |
| | | GVLSATILYEILLGKATLYAVLVSTLVVMAMVKRKNS |
| 147 | PIK3CA | TISGNEY |
| 148 | PIK3CA | GLKNN |
| 149 | PIK3CA | CIVRVAGSARQLTF |
| 150 | PIK3CA | SGHDT |
| 151 | PIK3CA | YYEEEE |
| 152 | PIK3CA | CASSFGTATYEQYF |
| 153 | PIK3CA | MRLVARVTVFLTFGTIIDAKTTQPPSMDCAEGRAANLPCNHSTISG |
| | | NEYVYWYRQIHSQGPQYIIHGLKNNETNEMASLIITEDRKSSTLILP |
| | | HATLRDTAVYYCIVRVAGSARQLTFGSGTQLTVLP |
| 154 | PIK3CA | MRLVARVTVFLTFGTIIDAKTTQPPSMDCAEGRAANLPCNHSTISG |
| | | NEYVYWYRQIHSQGPQYIIHGLKNNETNEMASLIITEDRKSSTLILP |
| | | HATLRDTAVYYCIVRVAGSARQLTFGSGTQLTVLPNIQNPEPAVY |
| | | QLKDPRSQDSTLCLFTDFDSQINVPKTMESGTFITDKCVLDMKAM |
| | | DSKSNGAIAWSNQTSFTCQDIFKETNATYPSSDVPCDATLTEKSFE |
| | | TDMNLNFQNLLVIVLRILLLKVAGFNLLMTLRLWSS |
| 155 | PIK3CA | MGPGLLCWALLCLLGAGLVDAGVTQSPTHLIKTRGQQVTLRCSP |
| | | KSGHDTVSWYQQALGQGPQFIFQYYEEEERQRGNFPDRFSGHQFP |
| | | NYSSELNVNALLLGDSALYLCASSFGTATYEQYFGPGTRLTVT |
| 156 | PIK3CA | MGPGLLCWALLCLLGAGLVDAGVTQSPTHLIKTRGQQVTLRCSP |
| | | KSGHDTVSWYQQALGQGPQFIFQYYEEEERQRGNFPDRFSGHQFP |
| | | NYSSELNVNALLLGDSALYLCASSFGTATYEQYFGPGTRLTVTED |
| | | LRNVTPPKVSLFEPSKAEIANKQKATLVCLARGFFPDHVELSWWV |
| | | NGKEVHSGVCTDPQAYKESNYSYCLSSRLRVSATFWHNPRNHFR |
| | | CQVQFHGLSEEDKWPEGSPKPVTQNISAEAWGRADCGITSASYQQ |
| | | GVLSATILYEILLGKATLYAVLVSTLVVMAMVKRKNS |
| 157 | PIK3CA | DSASNY |
| 158 | PIK3CA | IRSNVGE |
| 159 | PIK3CA | CAASIPGTASKLTF |
| 160 | PIK3CA | MNHEY |
| 161 | PIK3CA | SMNVEV |
| 162 | PIK3CA | CASSPYRQGSYGYTF |
| 163 | PIK3CA | MTSIRAVFIFLWLQLDLVNGENVEQHPSTLSVQEGDSAVIKCTYS |
| | | DSASNYFPWYKQELGKGPQLIIDIRSNVGEKKDQRIAVTLNKTAK |
| | | HFSLHITETQPEDSAVYFCAASIPGTASKLTFGTGTRLQVTL |
| 164 | PIK3CA | MTSIRAVFIFLWLQLDLVNGENVEQHPSTLSVQEGDSAVIKCTYS |
| | | DSASNYFPWYKQELGKGPQLIIDIRSNVGEKKDQRIAVTLNKTAK |
| | | HFSLHITETQPEDSAVYFCAASIPGTASKLTFGTGTRLQVTLNIQNP |
| | | EPAVYQLKDPRSQDSTLCLFTDFDSQINVPKTMESGTFITDKCVLD |
| | | MKAMDSKSNGAIAWSNQTSFTCQDIFKETNATYPSSDVPCSATLT |
| | | EKSFETDMNLNFQNLLVIVLRILLLKVAGFNLLMTLRLWSS |
| 165 | PIK3CA | MGPQLLGYVVLCLLGAGPLEAQVTQNPRYLITVTGKKLTVTCSQ |
| | | NMNHEYMSWYRQDPGLGLRQIYYSMNVEVTDKGDVPEGYKVSR |
| | | KEKRNFPLILESPSPNQTSLYFCASSPYRQGSYGYTFGSGTRLTVV |
| 166 | PIK3CA | MGPQLLGYVVLCLLGAGPLEAQVTQNPRYLITVTGKKLTVTCSQ |
| | | NMNHEYMSWYRQDPGLGLRQIYYSMNVEVTDKGDVPEGYKVSR |
| | | KEKRNFPLILESPSPNQTSLYFCASSPYRQGSYGYTFGSGTRLTVV |
| | | EDLRNVTPPKVSLFEPSKAEIANKQKATLVCLARGFFPDHVELSW |
| | | WVNGKEVHSGVCTDPQAYKESNYSYCLSSRLRVSATFWHNPRN |
| | | HFRCQVQFHGLSEEDKWPEGSPKPVTQNISAEAWGRADCGITSAS |
| | | YQQGVLSATILYEILLGKATLYAVLVSTLVVMAMVKRKNS |
| 167 | PIK3CA | NTAFDY |
| 168 | PIK3CA | IRPDVSE |
| 169 | PIK3CA | CAASTGNFNKFYF |
| 170 | PIK3CA | SGHNS |
| 171 | PIK3CA | FNNNVP |
| 172 | PIK3CA | CASNRQGTVTEAFF |
| 173 | PIK3CA | MDKILGASFLVLWLQLCWVSGQQKEKSDQQQVKQSPQSLIVQKG |
| | | GISIINCAYENTAFDYFPWYQQFPGKGPALLIAIRPDVSEKKEGRFT |
| | | ISFNKSAKQFSLHIMDSQPGDSATYFCAASTGNFNKFYFGSGTKLN |
| | | VKP |
| 174 | PIK3CA | MDKILGASFLVLWLQLCWVSGQQKEKSDQQQVKQSPQSLIVQKG |
| | | GISIINCAYENTAFDYFPWYQQFPGKGPALLIAIRPDVSEKKEGRFT |
| | | ISFNKSAKQFSLHIMDSQPGDSATYFCAASTGNFNKFYFGSGTKLN |
| | | VKPNIQNPEPAVYQLKDPRSQDSTLCLFTDFDSQINVPKTMESGTF |
| | | ITDKCVLDMKAMDSKSNGAIAWSNQTSFTCQDIFKETNATYPSSD |
| | | VPCDATLTEKSFETDMNLNFQNLLVIVLRILLLKVAGENLLMTLRL |
| | | WSS |
| 175 | PIK3CA | MDSWTFCCVSLCILVAKHTDAGVIQSPRHEVTEMGQEVTLRCKPI |
| | | SGHNSLFWYRQTMMRGLELLIYFNNNVPIDDSGMPEDRFSAKMP |
| | | NASFSTLKIQPSEPRDSAVYFCASNRQGTVTEAFFGQGTRLTVV |
| 176 | PIK3CA | MDSWTFCCVSLCILVAKHTDAGVIQSPRHEVTEMGQEVTLRCKPI |
| | | SGHNSLFWYRQTMMRGLELLIYFNNNVPIDDSGMPEDRFSAKMP |
| | | NASFSTLKIQPSEPRDSAVYFCASNRQGTVTEAFFGQGTRLTVVED |
| | | LRNVTPPKVSLFEPSKAEIANKQKATLVCLARGFFPDHVELSWWV |
| | | NGKEVHSGVCTDPQAYKESNYSYCLSSRLRVSATFWHNPRHFRC |
| | | QVQFHGLSEEDKWPEGSPKPVTQNISAEAWGRADCGITSASYQQ |
| | | GVLSATILYEILLGKATLYAVLVSTLVVMAMVKRKNS |
| 177 | P53 | KTYPVQLWV |
| 178 | P53 | KLYPVQLWV |
| 179 | P53 | KMYPVQLWV |
| 180 | P53 | KMYPVQLWL |
| 181 | P53 | KLYPVQLWI |
| 182 | P53 | GLAPPQLLI |
| 183 | P53 | GLAPPQLLV |
| 184 | P53 | GMAPPQLLV |
| 185 | P53 | GLAPPQYLI |
| 186 | P53 | GLAPPQYLV |
| 187 | P53 | GMAPPQYLV |
| 188 | P53 | ALNNMFCQL |
| 189 | P53 | ALNNMFCQV |
| 190 | P53 | ANINNMFCQV |
| 191 | P53 | NMFCQLAKT |
| 192 | P53 | NLFCQLAKV |
| 193 | P53 | QLWVDSTPL |
| 194 | P53 | QLWVDSTPI |
| 195 | P53 | QLWVDSTPV |
| 196 | P53 | RLILTIITL |
| 197 | P53 | RLILTIITV |
| 198 | P53 | YQGSYGFLL |
| 199 | P53 | YQGSYGFLI |
| 200 | P53 | YQGSYGFLV |
| 201 | P53 | SVTCTYFPA |
| 202 | P53 | SMTCTYFPL |
| 203 | P53 | SLTCTYFPV |
| 204 | P53 | SMTCTYFPV |
| 205 | P53 | SLTCTYFPL |
| 206 | P53 | SMTCTYFPI |
| 207 | P53 | LLGRNSFEM |
| 208 | P53 | LLGRNSFEL |
| 209 | P53 | LLGRNSFEI |
| 210 | P53 | LLGRNSFEV |
| 211 | P53 | VVPCEPPEV |
| 212 | P53 | VLPCEPPEV |
| 213 | P53 | VMPCEPPEV |
| 214 | KRAS | METLLGLLILWLQLQWVSSKQEVTQIPAALSVPEGENLVLNCSFD |
| | | SAIYNLQWFRQDPGKGLTSLLLIQSSQREQTSGRLNASLDKSSGRS |
| | | TLYIAASQPGDSATYLCAPNDYKLSFGAGTTVTVRANIQNPDPAV |
| | | YQLRDS |
| 215 | KRAS | MGCRLLCWVLLCLLGAGPVKAGVTQTPRYLIKTRGQQVTLSCSPI |
| | | SGHRSVSWYQQTPGQGLQFLFEYFSETQRNKGNFPGRFSGRQFSN |
| | | SRSEMNVSTLELGDSALYLCASSFSSDNEQFFGPGTRLTVLEDLKN |
| | | VFPPEVAVFE |
| 216 | KRAS | METLLGVSLVILWLQLARVNSQQGEEDPQALSIQEGENATMNCSY |
| | | KTSINNLQWYRQNSGRGLVHLILIRSNEREKHSGRLRVTLDTSKKS |
| | | SSLLITASRAADTASYFCATDSNDYKLSFGAGTTVTVRANIQNPDP |
| | | AVYQLRDS |
| 217 | KRAS | MDIWLLCWVVLGFLGTDHTGAGVSQSPRYKVTKRGQDVALRCD |
| | | PISGHVSLYWYRQALGQGPEFLTYFNYEAQQDKSGLPNDRFSAER |
| | | PEGSISTLTIQRTEQRDSAMYRCASSWGGELFFGEGSRLTVLEDLK |
| | | NVFPPEVAVFE |
| 218 | KRAS | MACPGFLWALVISTCLEFSMAQTVTQSQPEMSVQEAETVTLSCTY |
| | | DTSESDYYLFWYKQPPSRQMILVIRQEAYKQQNATENRFSVNFQK |
| | | AAKSFSLKISDSQLGDAAMYFCAYRSDAGGTSYGKLTFGQGTILT |
| | | VHPNIQNPDPAVYQLRDS |
| 219 | KRAS | MSIGLLCWVLLCLLGAGSVETGVTQSPTHLIKTRGQQVTLRCSSQ |
| | | SGHNTVSWYQQALGQGPQFIFQYYREEENGRGNFPPRFSGLQFPN |
| | | YSSELNVNALELDDSALYLCASSPRTGGAGNTIYFGEGSWLTVVE |
| | | DLNKVFPPEVAVFE |
| 220 | KRAS | MASAPISMLAMLFTLSGLRAQSVAQPEDQVNVAEGNPLTVKCTY |
| | | SVSGNPYLFWYVQYPNRGLQFLLKYITGDNLVKGSYGFEAEFNKS |
| | | QTSFHLKKPSALVSDSALYFCAVRDIEVVNAGNMLTFGGGTRLM |
| | | VKPHIQNPDPAVYQLRDS |
| 221 | KRAS | MDIWLVCWVLLCLLGAGSVETGVTQSPTHLIKTRGQQVTLRCSSQ |
| | | SGHNTVSWYQQALGQGPQFIFQYYREEENGRGNFPPRFSGLQFPN |
| | | YSSELNVNALELDDSALYLCASTDRVRYEQFFGPGTRLTVLEDLK |
| | | NVFPPEVAVFE |
| 222 | KRAS | MEKNPLAAPLLILWFHLDCVSSILNVEQSPQSLHVQEGDSTNFTCS |
| | | FPSSNFYALHWYRWETAKSPEALFVMTLNGDEKKKGRISATLNT |
| | | KEGYSYLYIKGSQPEDSATYLCAFLGSGNTGKLIFGQGTTLQVKP |
| | | DIQNPDPAVYQLRDS |
| 223 | KRAS | MLLLLLLLGPGSGLGAVVSQHPSWVICKSGTSVKIECRSLDFQATT |
| | | MFWYRQFPKQSLMLMATSNEGSKATYEQGVEKDKFLINHASLTL |
| | | STLTVTSAHPEDSSFYICSAIRPSGGAFEQYFGPGTRLTVTEDLKNV |
| | | FPPEVAVFE |
| 224 | KRAS | MTSIRAVFIFLWLQLDLVNGENVEQHPSTLSVQEGDSAVIKCTYS |
| | | DSASNYFPWYKQELGKGPQLIIDIRSNVGEKKDQRIAVTLNKTAK |
| | | HFSLHITETQPEDSAVYFCAARATSGTYKYIFGTGTRLKVLANIQN |
| | | PDPAVYQLRDS |
| 225 | KRAS | MLLLLLLLGPGSGLGAVVSQHPSRVICKSGTSVKIECRSLDFQATT |
| | | MFWYRQFPKKSLMLMATSNEGSKATYEQGVEKDKFLINHASLTL |
| | | STLTVTSAHPEDSSFYICSASFSRDIETQYFGPGTRLLVLEDLKNVF |
| | | PPEVAVFE |
| 226 | KRAS | MWGAFLLYVSMKMGGTTGQNIDQPTEMTATEGAIVQINCTYQTS |
| | | GFNGLFWYQQHAGEAPTFLSYNVLDGLEEKGRFSSFLSRSKGYSY |
| | | LLLKELQMKDSASYLCAVRLGDAGNMLTFGGGTRLMVKPHIQNP |
| | | DPAVYQLRDS |
| 227 | KRAS | MGTRLLCCVAFCLLGAGPVDSGVTQTPKHLITATGQRVTLRCSPR |
| | | SGDLSVYWYQQSLDQGLQFLIQYYNGEERAKGNILERFSAQQFPD |
| | | LHSELNLSSLELGDSALYFCASSVGRGMNTEAFFGQGTRLTVVED |
| | | LNKVFPPEVAVFE |
| 228 | KRAS | MLLITSMLVLWMQLSQVNGQQVMQIPQYQHVQEGEDFTTYCNSS |
| | | TTLSNIQWYKQRPGGHPVFLIQLVKSGEVKKQKRLTFQFGEAKKN |
| | | SSLHITATQTTDVGTYFCADNAGNMLTFGGGTRLMVKPHIQNPDP |
| | | AVYQLRDS |
| 229 | KRAS | MLLLLLLLGPAGSGLGAVVSQHPSWVICKSGTSVKIECRSLDFQA |
| | | TTMFWYRQFPKQSLMLMATSNEGSKATYEQGVEKDKFLINHASL |
| | | TLSTLTVTSAHPEDSSFYICSARDPGRNYGYTFGSGTRLTVVEDLN |
| | | KVFPPEVAVFE |
| 230 | KRAS | MLLITSMLVLWMQLSQVNGQQVMQIPQYQHVQEGEDFTTYCNSS |
| | | TTLSNIQWYKQRPGGHPVFLIQLVKSGEVKKQKRLTFQFGEAKKN |
| | | SSLHITATQTTDVGTYFCGDNAGNMLTFGGGTRLMVKPHIQNPDP |
| | | AVYQLRDS |
| 231 | KRAS | MLLLLLLLGPGSGLGAVVSQHPSWVICKSGTSVKIECRSLDFQATT |
| | | MFWYRQFPKQSLMLMATSNEGSKATYEQGVEKDKFLINHASLTL |
| | | STLTVTSAHPEDSSFYICSARDEDRPYGYTFGSGTRLTVVEDLNKV |
| | | FPPEVAVFE |
| 232 | KRAS | MLLLLVPVLEVIFTLGGTRAQSVTQLGSHVSVSEGALVLLRCNYS |
| | | SSVPPYLFWYVQYPNQGLQLLLKYTSAATLVKGINGFEAEFKKSE |
| | | TSFHLTKPSAHMSDAAEYFCAVSDGGATNKLIFGTGTLLAVQPNI |
| | | QNPDPAVYQLRDS |
| 233 | KRAS | MGTRLLCCVVLCLLGANTVDGGITQSPKYLFRKEGQNVTLSCEQ |
| | | NLNHDAMYWYRQDPGQGLRLIYYSQIVNDFQKGDIAEGYSVSRE |
| | | KKESFPLTVTSAQKNPTAFYLCASSSGGRASFTQYFGPGTRLTVLE |
| | | DLKNVFPPEVAVFE |
| 234 | KRAS | MNYSPGLVSLILLLLGRTRGNSVTQMEGPVTLSEEAFLTINCTYTA |
| | | TGYPSLFWYVQYPGEGLQLLLKATKADDKGSNKGFEATYRKETT |
| | | SFHLEKGSVQVSDSAVYFCALSDPNNQGGKLIFGQGTELSVKPNI |
| | | QNPDPAVYQLRDS |
| 235 | KRAS | MGTRLLCCVVFCLLQAGPLDTAVSQTPKYLVTQMGNDKSIKCEQ |
| | | NLGHDTMYWYKQDSKKFLKIMFSYNNKELIINETVPNRFSPKSPD |
| | | KAHLNLHINSLELGDSAVYFCASSQVRGTTEAFFGQGTRLTVVED |
| | | LNKVFPPEVAVFE |
| 236 | KRAS | MKKLLAMILWLQLDRLSGELKVEQNPLFLSMQEGKNYTIYCNYS |
| | | TTSDRLYWYRQDPGKSLESLFVLLSNGAVKQEGRLMASLDTKAR |
| | | LSTLHITAAVHDLSATYFCAEPNTDKLIFGTGTRLQVFPNIQNPDP |
| | | AVYQLRDS |
| 237 | KRAS | MGPGLLHWMALCLLGTGHGDAMVIQNPRYQVTQFGKPVTLSCS |
| | | QTLNHNVMYWYQQKSSQAPKLLFHYYDKDFNNEADTPDNFQSR |
| | | RPNTSFCFLDIRSPGLGDTAMYLCATNYDRGYEQYFGPGTRLTVT |
| | | EDLKNVFPPEVAVFE |
| 238 | KRAS | MKSLRVLLVILWLQLSWVWSQQKEVEQNSGPLSVPEGAIASLNCT |
| | | YSDRGSQSFFWYRQYSGKSPELIMFIYSNGDKEDGRFTAQLNKAS |
| | | QYVSLLIRDSQPSDSATYLCAVWATGNQFYFGTGTSLTVIPNIQNP |
| | | DPAVYQLRDS |
| 239 | KRAS | MGPGLLHWMALCLLGTGHGDAMVIQNPRYQVTQFGKPVTLSCS |
| | | QTLNHNVMYWYQQKSSQAPKLLFHYYDKDFNNEADTPDNFQSR |
| | | RPNTSFCFLDIRSPGLGDTAMYLCATSFDRGYEQYFGPGTRLTVTE |
| | | DLKNVFPPEVAVFE |
| 240 | KRAS | MISLRVLLVILWLQLSWVWSQRKEVEQDPGPFNVPEGATVAFNC |
| | | TYSNSASQSFFWYRQDCRKEPKLLMSVYSSGNEDGRFTAQLNRA |
| | | SQYISLLIRDSKLSDSATYLCVVNERNSGYALNFGKGTSLLVTPHI |
| | | QNPDPAVYQLRDS |
| 241 | KRAS | MASLLFFCGAFYLLGTGSMDADVTQTPRNRITKTGKRIMLECSQT |
| | | KGHDRMYWYRQDPGLGLRLIYYSFDVKDINKGEISDGYSVSRQA |
| | | QAKFSLSLESAIPNQTALYFCATSGPDSNQPQHFGDGTRLSILEDL |
| | | NKVFPPEVAVFE |
| 242 | KRAS | MLLEHLLIILWMQLTWVSGQQLNQSPQSMFIQEGEDVSMNCTSSS |
| | | IFNTWLWYKQEPGEGPVLLIALYKAGELTSNGRLTAQFGITRKDSF |
| | | LNISASIPSDVGIYFCAGQLRYGGATNKLIFGTGTLLAVQPNIQNPD |
| | | PAVYQLRDS |
| 243 | KRAS | MGCRLLCCVAFCLLGAGPVDSGVTQTPKHLITATGQRVTLRCSPR |
| | | SGDLSVYWYQQSLDQGLQFLIHYYNGEERAKGNILERFSAQQFPD |
| | | LHSELNLSSLELGDSALYFCASSVGGASYEQYFGPGTRLTVTEDL |
| | | KNVFPPEVAVFE |
| 244 | KRAS | MRQVARVIVFLTLSTLSLAKTTQPISMDSYEGQEVNITCSHNNIAT |
| | | NDYITWYQQFPSQGPRFIIQGYKTKVTNEVASLFIPADRKSSTLSLP |
| | | RVSLSDTAVYYCLVGPTYNQGGKLIFGQGTELSVKPNIQNPDPAV |
| | | YQLRDS |
| 245 | KRAS | MLLLLLLLGPGSGLGAVVSQHPSRVICKSGTSVKIECRSLDFQATT |
| | | MFWYRQFPKKSLMLMATSNEGSKATYEQGVEKDKFLINHASLTL |
| | | STLTVTSAHPEDSSFYICSARTAEVELFFGSGTQLSVLEDLNKVFPP |
| | | EVAVFE |
| 246 | KRAS | MKTFAGFSFLFLWLQLDCMSRGEDVEQSLFLSVREGDSSVINCTY |
| | | TDSSSTYLYWYKQEPGAGLQLLTYIFSNMDMKQDQRLTVLLNKK |
| | | DKHLSLRIADTQTGDSAIYFCAVASWGSGSARQLTFGSGTQLTVL |
| | | PDIQNPDPAVYQLRDS |
| 247 | KRAS | MGTRLLCWALLCLLGAGLVDAGVTQSPTHLIKTRGQQVTLRCSP |
| | | KSGHDTVSWYQQALGQGPQFIFQYYEEEERQRGNFPDRFSGHQFP |
| | | NYSSELNVNALLLGDSALYLCASSLDAGYEQYFGPGTRLTVTEDL |
| | | KNVFPPEVAVFE |
| 248 | KRAS | METVLQVLLGILGFQAAWVSSQELEQSPQSLIVQEGKNLTINCTSS |
| | | KTLYGLYWYKQKYGEGLIFLMMLQKGGEEKSHEKITAKLDEKKQ |
| | | QSSLHITASQPSHAGIYLCGADMLDYQKVTFGIGTKLQVIPNIQNP |
| | | DPAVYQLRDS |
| 249 | KRAS | MGSWTLCCVSLCILVAKHTDAGVIQSPRHEVTEMGQEVTLRCKPI |
| | | SGHDYLFWYRQTMMRGLELLIYFNNNVPIDDSGMPEDRFSAKMP |
| | | NASFSTLKIQPSEPRDSAVYFCASRRGGSEQYFGPGTRLTVTEDLK |
| | | NVFPPEVAVFE |
| 250 | KRAS | MSLSSLLKVVTASLWLGPGIAQKITQTQPGMFVQEKEAVTLDCTY |
| | | DTSDPSYGLFWYKQPSSGEMIFLIYQGSYDQQNATEGRYSLNFQK |
| | | ARKSANLVISASQLGDSAMYFCAMREGQDSSYKLIFGSGTRLLVR |
| | | PDIQNPDPAVYQLRDS |
| 251 | KRAS | MGTRLLCCVAFCLLGAGPVDSGVTQTPKHLITATGQRVTLRCSPR |
| | | SGDLSVYWYQQSLDQGLQFLIQYYNGEERAKGNILERFSAQQFPD |
| | | LHSELNLSSLELGDSALYFCASSVLGPNTEAFFGQGTRLTVVEDLN |
| | | KVFPPEVAVFE |
| 252 | KRAS | MLTASLLRAVIASICVVSSMAQKVTQAQTEISVVEKEDVTLDCVY |
| | | ETRDTTYYLFWYKQPPSGELVFLIRRNSFDEQNEISGRYSWNFQKS |
| | | TSSFNFTITASQVVDSAVYFCALSNAGGTSYGKLTFGQGTILTVHP |
| | | NIQNPDPAVYQLRDS |
| 253 | KRAS | MGPQLLGYVVLCLLGAGPLEAQVTQNPRYLITVTGKKLTVTCSQ |
| | | NMNHEYMSWYRQDPGLGLRQIYYSMNVEVTDKGDVPEGYKVSR |
| | | KEKRNFPLILESPSPNQTSLYFCASSRRLPPPYGYTFGSGTRLTVVE |
| | | DLNKVFPPEVAVFE |
| 254 | KRAS | MLTASLLRAVIASICVVSSMAQKVTQAQTEISVVEKEDVTLDCVY |
| | | ETRDTTYYLFWYKQPPSGELVFLIRRNSFDEQNEISGRYSWNFQKS |
| | | TSSFNFTITASQVVDSAVYFCALSESSNTGKLIFGQGTTLQVKPDIQ |
| | | NPDPAVYQLRDS |
| 255 | KRAS | MGPQLLGYVVLCLLGAGPLEAQVTQNPRYLITVTGKKLTVTCSQ |
| | | NMNHEYMSWYRQDPGLGLRQIYYSMNVEVTDKGDVPEGYKVSR |
| | | KEKRNFPLILESPSPNQTSLYFCASSRREPPPYGYTFGSGTRLTVVE |
| | | DLNKVFPPEVAVFE |
| 256 | KRAS | MSLSSLLKVVTASLWLGPGIAQKITQTQPGMFVQEKEAVTLDCTY |
| | | DTSDPSYGLFWYKQPSSGEMIFLIYQGSYDQQNATEGRYSLNFQK |
| | | ARKSANLVISASQLGDSAMYFCAMREIRDSSYKLIFGSGTRLLVRP |
| | | DIQNPDPAVYQLRDS |
| 257 | KRAS | MGTRLLCCVAFCLLGAGPVDSGVTQTPKHLITATGQRVTLRCSPR |
| | | SGDLSVYWYQQSLDQGLQFLIQHYNGEERAKGNILERFSAQQFPD |
| | | LHSELNLSSLELGDSALYFCASSPAGPNTEAFFGQGTRLTVVEDLN |
| | | KVFPPEVAVFE |
| 258 | RAS/ | gaggtgcagctgttggagtctgggggaggcttggtacagcctggggggtccctgagactctcctgtgca |
| | P53 | gcctctggattcacctttagcagctatgccatgagctgggtccgccaggctccagggaaggggctggag |
| | | tgggtctcagctattagtggtagtggtggtagcacatactacgcagactccgtgaagggccggttcaccat |
| | | ctccagagacaattccaagaacacgctgtatctgcaaatgaacagcctgagagccgaggacacggcgg |
| | | tgtactactgcgcaagagccgagatgggagccgtattcgacatatggggtcagggtacaatggtcaccg |
| | | tctcctca |
| 259 | RAS/ | caggtgcagctggtggagtctgggggaggcgtggtccagcctgggaggtccctgagactctcctgtgc |
| | P53 | agcgtctggattcaccttcagtagctatggcatgcactgggtccgccaggctccaggcaaggggctgga |
| | | gtgggtggcagttatatcgtatgatggaagtaataaatactatgcagactccgtgaagggccgattcacca |
| | | tctccagagacaattccaagaacacgctgtatctgcaaatgaacagcctgagagccgaggacacggcg |
| | | gtgtactactgcgccagagacggtacttatctaggtggtctctggtacttcgacttatgggggagaggtac |
| | | cttggtcaccgtctcctca |
| 260 | RAS/ | caggtgcagctggtgcagtctggggctgaggtgaagaagcctggggcctcagtgaaggtttcctgcaa |
| | P53 | ggcatctggatacaccttcaccagctactatatgcactgggtgcgacaggcccctggacaagggcttga |
| | | gtggatgggaataatcaaccctggtggtggtagcacaagctacgcacagaagttccagggcagagtca |
| | | ccatgaccagggacacgtccacgagcacagtctacatggagctgagcagcctgagatctgaggacac |
| | | ggcggtgtactactgcgccagagagagttggccaatggacgtatggggccagggaacaactgtcaccg |
| | | tctcctca |
| 261 | RAS/ | cagctgcagctgcaggagtcgggcccaggactggtgaagccttcggagaccctgtccctcacctgcac |
| | P53 | tgtctctggtggctccatcagcagtagtagttactactggggctggatccgccagcccccagggaaggg |
| | | gctggagtggattgggagtatctcctatagtgggagcacctactacaacccgtccctcaagagtcgagtc |
| | | accatatccgtagacacgtccaagaaccagttctccctgaagctgagttctgtgaccgccgcagacacg |
| | | gcggtgtactactgcgccagaggcaggggatatgcaaccagcttagccttcgatatctggggtcagggt |
| | | acaatggtcaccgtctcctca |
| 262 | RAS/ | gaggtgcagctggtggagtctgggggaggcttggtacagcctggggggtccctgagactctcctgtgc |
| | P53 | agcctctggattcaccttcagtagctatagcatgaactgggtccgccaggctccagggaaggggctgga |
| | | gtgggtttcaaccattagtagtagtagtagtaccatatactacgcagactctgtgaagggccgattcaccat |
| | | ctccagagacaatgccaagaactcactgtatctgcaaatgaacagcctgagagctgaggacacggcgg |
| | | tgtactactgcgccagaggttctcaggagcacctgattttcgattattggggacagggtacattggtcacc |
| | | gtctcctca |
| 263 | RAS/ | caggtgcagctggtggagtctgggggaggcgtggtccagcctgggaggtccctgagactctcctgtgc |
| | P53 | agcgtctggattcaccttcagtagctatggcatgcactgggtccgccaggctccaggcaaggggctgga |
| | | gtgggtggcagttatatcgtatgatggaagtaataaatactatgcagactccgtgaagggccgattcacca |
| | | tctccagagacaattccaagaacacgctgtatctgcaaatgaacagcctgagagccgaggacacggcg |
| | | gtgtactactgcgccagaactgacttctggagcggatcccctccaggcttagattactggggacagggta |
| | | cattggtcaccgtctcctca |
| 264 | RAS/ | caggtgcagctggtgcagtctggggctgaggtgaagaagcctgggtcctcggtgaaggtctcctgcaa |
| | P53 | ggcttctggaggcaccttcagcagctatgctatcagctgggtgcgacaggcccctggacaagggcttga |
| | | gtggatgggagggatcatccctatctttggtacagcaaactacgcacagaagttccagggcagagtcac |
| | | gattaccgcggacgaatccacgagcacagcctacatggagctgagcagcctgagatctgaggacacg |
| | | gcggtgtactactgcgccagaactcctgaatactcctccagcatatggcactattactacggcatggacgt |
| | | atggggccagggaacaactgtcaccgtctcctca |
| 265 | RAS/ | caggtgcagctggtggagtctgggggaggcgtggtccagcctgggaggtccctgagactctcctgtgc |
| | P53 | agcgtctggattcaccttcagtagctatggcatgcactgggtccgccaggctccaggcaaggggctgga |
| | | gtgggtggcagttatatcgtatgatggaagtaataaatactatgcagactccgtgaagggccgattcacca |
| | | tctccagagacaattccaagaacacgctgtatctgcaaatgaacagcctgagagccgaggacacggcg |
| | | gtgtactactgcgtcaaggggccgttgcaggagccgccatacgattatggaatggacgtatggggccag |
| | | ggaacaactgtcaccgtctcctca |
| 266 | RAS/ | gaaattgtgttgacacagtctccagccaccctgtctttgtctccaggggaaagagccaccctctcctgcag |
| | P53 | ggccagtcagagtgttagcaggtacttagcctggtaccaacagaaacctggccaggctcccaggctcct |
| | | catctatgatgcatccaacagggccactggcatcccagccaggttcagtggcagtgggtctgggacaga |
| | | cttcactctcaccatcagcagcctagagcctgaagattttgcagtttattactgtcagcagagaatctcctgg |
| | | cctttcacttttggcggagggaccaaggttgagatcaaa |
| 267 | RAS/ | gatattgtgatgactcagtctccactctccctgcccgtcacccctggagagccggcctccatctcctgcag |
| | P53 | gtctagtcagagcctcctgcatagtaatggatacaactatttggattggtacctgcagaagccagggcagt |
| | | ctccacagctcctgatctatttgggttctaatcgggcctccggggtccctgacaggttcagtggcagtgga |
| | | tcaggcacagattttacactgaaaatcagcagagtggaggctgaggatgttggggtttattactgcatgca |
| | | gggactcggcctccctctcacttttggcggagggaccaaggttgagatcaaa |
| 268 | RAS/ | gaaatagtgatgacgcagtctccagccaccctgtctgtgtctccaggggaaagagccaccctctcctgca |
| | P53 | gggccagtcagagtgttagcagcaacttagcctggtaccagcagaaacctggccaggctcccaggctc |
| | | ctcatctatggtgcatccaccagggccactggtatcccagccaggttcagtggcagtgggtctgggaca |
| | | gagttcactctcaccatcagcagcctgcagtctgaagattttgcagtttattactgtcagcagtacgccgcct |
| | | accctacttttggggagggaccaaggttgagatcaaa |
| 269 | RAS/ | gaaattgtgttgacacagtctccagccaccctgtctttgtctccaggggaaagagccaccctctcctgcag |
| | P53 | ggccagtcagagtgttagcagctacttagcctggtaccaacagaaacctggccaggctcccaggctcct |
| | | catctatgatgcatccaacagggccactggcatcccagccaggttcagtggcagtgggtctgggacaga |
| | | cttcactctcaccatcagcagcctagagcctgaagattttgcagtttattactgtcagcagagacacgtctg |
| | | gcctcctacttttggcggagggaccaaggttgagatcaaa |
| 270 | RAS/ | gaaattgtgttgacacagtctccagccaccctgtctttgtctccaggggaaagagccaccctctcctgcag |
| | P53 | ggccagtcagagtgttagcaggtacttagcctggtaccaacagaaacctggccaggctcccaggctcct |
| | | catctatgatgcatccaacagggccactggcatcccagccaggttcagtggcagtgggtctgggacaga |
| | | cttcactctcaccatcagcagcctagagcctgaagattttgcagtttattactgtcagcagagattctactac |
| | | ccttggacttttggcggagggaccaaggttgagatcaaa |
| 271 | RAS/ | gacatccagttgacccagtctccatcttccgtgtctgcatctgtaggagacagagtcaccatcacttgtcgg |
| | P53 | gcgagtcagggtattagcagctggttagcctggtatcagcagaaaccagggaaagcccctaagctcctg |
| | | atctatggtgcatccagtttgcaaagtggggtcccatcaaggttcagcggcagtggatctgggacagattt |
| | | cactctcaccatcagcagcctgcagcctgaagattttgcaacttattactgtcagcagatatacaccttccct |
| | | ttcacttttggggagggaccaaggttgagatcaaa |
| 272 | RAS/ | gacatcgtgatgacccagtctccagactccctggctgtgtctctgggcgagagggccaccatcaactgc |
| | P53 | aagtccagccagagtgttttatacagctccaacaataagaactacttagcttggtaccagcagaaaccag |
| | | gacagcctcctaagctgctcatttactgggcatctacccgggaatccggggtccctgaccgattcagtgg |
| | | cagcgggtctgggacagatttcactctcaccatcagcagcctgcaggctgaagatgtggcagtttattact |
| | | gtcagcagttcgcccacactcctttcacttttggcggagggaccaaggttgagatcaaa |
| 273 | RAS/ | gaaatagtgatgacgcagtctccagccaccctgtctgtgtctccaggggaaagagccaccctctcctgca |
| | P53 | gggccagtcagagtgttagcagcaacttagcctggtaccagcagaaacctggccaggctcccaggctc |
| | | ctcatctatagcgcatccaccagggccactggtatcccagccaggttcagtggcagtgggtctgggaca |
| | | gagttcactctcaccatcagcagcctgcagtctgaagattttgcagtttattactgtcagcagcaccacgtct |
| | | ggcctctcacttttggcggagggaccaaggttgagatcaaa |
| 274 | RAS/ | EVQLLESGGGLVQPGGSLRLSCAASGFTFSSYAMSWVRQAPGKG |
| | P53 | LEWVSAISGSGGSTYYADSVKGRFTISRDNSKNTLYLQMNSLRAE |
| | | DTAVYYCARAEMGAVFDIWGQGTMVTVSS |
| 275 | RAS/ | QVQLVESGGGVVQPGRSLRLSCAASGFTFSSYGMHWVRQAPGKG |
| | P53 | LEWVAVISYDGSNKYYADSVKGRFTISRDNSKNTLYLQMNSLRA |
| | | EDTAVYYCARDGTYLGGLWYFDLWGRGTLVTVSS |
| 276 | RAS/ | QVQLVQSGAEVKKPGASVKVSCKASGYTFTSYYMHWVRQAPGQ |
| | P53 | GLEWMGIINPGGGSTSYAQKFQGRVTMTRDTSTSTVYMELSSLRS |
| | | EDTAVYYCARESWPMDVWGQGTTVTVSS |
| 277 | RAS/ | QLQLQESGPGLVKPSETLSLTCTVSGGSISSSSYYWGWIRQPPGKG |
| | P53 | LEWIGSISYSGSTYYNPSLKSRVTISVDTSKNQFSLKLSSVTAADTA |
| | | VYYCARGRGYATSLAFDIWGQGTMVTVSS |
| 278 | RAS/ | EVQLVESGGGLVQPGGSLRLSCAASGFTFSSYSMNWVRQAPGKG |
| | P53 | LEWVSTISSSSSTIYYADSVKGRFTISRDNAKNSLYLQMNSLRAED |
| | | TAVYYCARGSQEHLIFDYWGQGTLVTVSS |
| 279 | RAS/ | QVQLVESGGGVVQPGRSLRLSCAASGFTFSSYGMHWVRQAPGKG |
| | P53 | LEWVAVISYDGSNKYYADSVKGRFTISRDNSKNTLYLQMNSLRA |
| | | EDTAVYYCARTDFWSGSPPGLDYWGQGTLVTVSS |
| 280 | RAS/ | QVQLVQSGAEVKKPGSSVKVSCKASGGTFSSYAISWVRQAPGQG |
| | P53 | LEWMGGIIPIFGTANYAQKFQGRVTITADESTSTAYMELSSLRSED |
| | | TAVYYCARTPEYSSSIWHYYYGMDVWGQGTTVTVSS |
| 281 | RAS/ | QVQLVESGGGVVQPGRSLRLSCAASGFTFSSYGMHWVRQAPGKG |
| | P53 | LEWVAVISYDGSNKYYADSVKGRFTISRDNSKNTLYLQMNSLRA |
| | | EDTAVYYCVKGPLQEPPYDYGMDVWGQGTTVTVSS |
| 282 | RAS/ | EIVLTQSPATLSLSPGERATLSCRASQSVSRYLAWYQQKPGQAPRL |
| | P53 | LIYDASNRATGIPARFSGSGSGTDFTLTISSLEPEDFAVYYCQQRIS |
| | | WPFTFGGGTKVEIK |
| 283 | RAS/ | DIVMTQSPLSLPVTPGEPASISCRSSQSLLHSNGYNYLDWYLQKPG |
| | P53 | QSPQLLIYLGSNRASGVPDRFSGSGSGTDFTLKISRVEAEDVGVYY |
| | | CMQGLGLPLTFGGGTKVEIK |
| 284 | RAS/ | EIVMTQSPATLSVSPGERATLSCRASQSVSSNLAWYQQKPGQAPR |
| | P53 | LLIYGASTRATGIPARFSGSGSGTEFTLTISSLQSEDFAVYYCQQYA |
| | | AYPTFGGGTKVEIK |
| 285 | RAS/ | EIVLTQSPATLSLSPGERATLSCRASQSVSSYLAWYQQKPGQAPRL |
| | P53 | LIYDASNRATGIPARFSGSGSGTDFTLTISSLEPEDFAVYYCQQRHV |
| | | WPPTFGGGTKVEIK |
| 286 | RAS/ | EIVLTQSPATLSLSPGERATLSCRASQSVSRYLAWYQQKPGQAPRL |
| | P53 | LIYDASNRATGIPARFSGSGSGTDFTLTISSLEPEDFAVYYCQQRFY |
| | | YPWTFGGGTKVEIK |
| 287 | RAS/ | DIQLTQSPSSVSASVGDRVTITCRASQGISSWLAWYQQKPGKAPK |
| | P53 | LLIYGASSLQSGVPSRFSGSGSGTDFTLTISSLQPEDFATYYCQQIY |
| | | TFPFTFGGGTKVEIK |
| 288 | RAS/ | DIVMTQSPDSLAVSLGERATINCKSSQSVLYSSNNKNYLAWYQQK |
| | P53 | PGQPPKLLIYWASTRESGVPDRFSGSGSGTDFTLTISSLQAEDVAV |
| | | YYCQQFAHTPFTFGGGTKVEIK |
| 289 | RAS/ | EIVMTQSPATLSVSPGERATLSCRASQSVSSNLAWYQQKPGQAPR |
| | P53 | LLIYSASTRATGIPARFSGSGSGTEFTLTISSLQSEDFAVYYCQQHH |
| | | VWPLTFGGGTKVEIK |
| 290 | RAS/ | caggtgcagctgcaggaatccggaccggggctggtgaagcccagcgagactctgagtctcacgtgtac |
| | P53 | agtttctggaggtagcattagctcctactattggtcatggataaggcagccccccgggaagggattggaat |
| | | ggatcggctatatttactacagtgggagcaccaattacaacccctcactgaagtctagagttacaatcagc |
| | | gttgacacctcaaagaatcagttcagtttgaaattgtctagcgtcacagcagctgatacagccgtctattatt |
| | | gtgtttctctggtctattgcggtggggattgttacagtggctttgactattgggggcagggtactctggttac |
| | | agtttcttcc |
| 291 | RAS/ | caggtacagctgcaggaatctgggcccggacttgtcaagccaagtcagacactttctcttacatgtaccgt |
| | P53 | gagcggcggaagtataagcagtggaggcttttactggtcttggatacggcagcacccaggcaaaggctt |
| | | ggagtggattggatacattcatcattcaggatctacacactataatccatcccttaagtcccgggtcaccatt |
| | | agcattgatacgtctaagaatctgttcagtctcaggctgtcctccgtcactgctgccgacacagccgtgtac |
| | | tactgcgcctccttggtttactgcggaggcgactgttatagcggctttgattattgggggcaggggaccct |
| | | cgtaaccgtgagctct |
| 292 | RAS/ | caggtccaactggtgcagtccggagccgaagtcaagaaaccaggtgcctccgttaaagtgagttgcaaa |
| | P53 | gtctctggatacactctgaccgagctctctatgcactgggtccggcaggcccccggcaagggattggaat |
| | | ggatgggcgggttcgatcctgaggacggagagactatctacgctcaaaaattccagggacgagtgact |
| | | gtgaccgaagacactagtaccgacactgcctacatggaactttcctctctgcgatcagaagataccgcag |
| | | tgtactactgtgctactgaatctaggggcattggatggccctacttcgattactggggtcagggaactctgg |
| | | tgactgtctccagc |
| 293 | RAS/ | caggtccagttggtcgaaagtggcggtggtgtagtgcagccgggccgcagtttgaggctttcctgtgcg |
| | P53 | gcttcaggctttactttttccagctatggaatgcactgggtgcggcaggcccccggcaaaggacttgagtg |
| | | ggtggccgtcatttcttatgacggatcagataagtactacgtggacagcgtcaagggcagattcaccatct |
| | | ctagggacaacagtaaaaatagactctacctccagatgaatagcctcagagctgaagacacggccgtct |
| | | actattgtgctcgggagcggtatagtggcagagactactgggggcagggcacactcgttacagtgagta |
| | | gc |
| 294 | RAS/ | gacatccagttgacacagagcccgagttccttgtccgcctccgtcggggatagagtgtcatttacctgtca |
| | P53 | ggcctctcaggatattaataactttctgaattggtatcagcaaaagcccggaaaggcacccaagctgttga |
| | | tttacgacgccagtaacctggagacaggcgtgccctcccggtttagtggtagcggaagcggtacggattt |
| | | tacctttactatcagctctctccaacccgaagacattgcaacctactattgtcaacaatatggaaacctgcct |
| | | tttacatttggcggcggcaccaaggtggagattaagcgg |
| 295 | RAS/ | gatatccagctcactcaaagcccctctagtctctctgcctcagtgggggatcgggtcagttttacttgtcaa |
| | P53 | gcttcacaggatatcaacaacttccttaattggtatcagcagaagccaggaaaagcacccaagctgctcat |
| | | ctatgatgcctcaaatttggagacgggtgttcccagtcgattctctgggtcagggtccgggaccgacttta |
| | | cgtttacgatctcctctctgcagcccgaagacatcgccacatactattgtcaacagtacggcaacttgccttt |
| | | cacatttggggggggactaaggttgaaatcaagagg |
| 296 | RAS/ | gatattcagatgactcaatctccttcttctctgtccgcttccgtgggcgatagagtgaccattacttgtaggg |
| | P53 | cgtcccagtcaatctccagttatttgaattggtatcagcagaagcccgggaaagcacctaagctgttgatc |
| | | agcggggcttctagcctgaagagtggggtaccttcacggttcagcggaagcggaagcggaaccgattt |
| | | caccctgactatcagcagcctgccacctgaggactttgcaacttactactgccaacagtcatacagcactc |
| | | cgatcactttcggccagggcacccggctcgaaatcaagcgc |
| 297 | RAS/ | gagattgttatgacccagagtcctgcgaccctctcagtcagccccggggagcgcgcaactttgtcttgca |
| | P53 | gagctagtcagtccgtgtcctctcttctgacatggtaccagcaaaagcccgggcaggctccgcgccttttg |
| | | atctttggggcttcaacaagagccactgggattcccgcacgattctctggctccgggagcggtactggttt |
| | | caccctgacgattagcagtctccagagcgaggacttcgccgtatactactgccagcagtacgatacgtgg |
| | | ccattcacttttggaccagggactaaagtggattttaagcgc |
| 298 | RAS/ | QVQLQESGPGLVKPSETLSLTCTVSGGSISSYYWSWIRQPPGKGLE |
| | P53 | WIGYIYYSGSTNYNPSLKSRVTISVDTSKNQFSLKLSSVTAADTAV |
| | | YYCVSLVYCGGDCYSGFDYWGQGTLVTVSS |
| 299 | RAS/ | QVQLQESGPGLVKPSQTLSLTCTVSGGSISSGGFYWSWIRQHPGK |
| | P53 | GLEWIGYIHHSGSTHYNPSLKSRVTISIDTSKNLFSLRLSSVTAADT |
| | | AVYYCASLVYCGGDCYSGFDYWGQGTLVTVSS |
| 300 | RAS/ | QVQLVQSGAEVKKPGASVKVSCKVSGYTLTELSMHWVRQAPGK |
| | P53 | GLEWMGGFDPEDGETIYAQKFQGRVTVTEDTSTDTAYMELSSLR |
| | | SEDTAVYYCATESRGIGWPYFDYWGQGTLVTVSS |
| 301 | RAS/ | QVQLVESGGGVVQPGRSLRLSCAASGFTFSSYGMHWVRQAPGKG |
| | P53 | LEWVAVISYDGSDKYYVDSVKGRFTISRDNSKNRLYLQMNSLRA |
| | | EDTAVYYCARERYSGRDYWGQGTLVTVSS |
| 302 | RAS/ | DIQLTQSPSSLSASVGDRVSFTCQASQDINNFLNWYQQKPGKAPK |
| | P53 | LLIYDASNLETGVPSRFSGSGSGTDFTFTISSLQPEDIATYYCQQYG |
| | | NLPFTFGGGTKVEIKR |
| 303 | RAS/ | DIQLTQSPSSLSASVGDRVSFTCQASQDINNFLNWYQQKPGKAPK |
| | P53 | LLIYDASNLETGVPSRFSGSGSGTDFTFTISSLQPEDIATYYCQQYG |
| | | NLPFTFGGGTKVEIKR |
| 304 | RAS/ | DIQMTQSPSSLSASVGDRVTITCRASQSISSYLNWYQQKPGKAPKL |
| | P53 | LISGASSLKSGVPSRFSGSGSGTDFTLTISSLPPEDFATYYCQQSYST |
| | | PITFGQGTRLEIKR |
| 305 | RAS/ | EIVMTQSPATLSVSPGERATLSCRASQSVSSLLTWYQQKPGQAPR |
| | P53 | LLIFGASTRATGIPARFSGSGSGTGFTLTISSLQSEDFAVYYCQQYD |
| | | TWPFTFGPGTKVDFKR |
| 306 | RAS/P53/ | ggaattccatatgagtcaacaaggagaagaagatcc |
| | PIK3CA | |
| 307 | RAS/P53/ | ttgtcagtcgacttagagtctctcagctggtacacg |
| | PIK3CA | |
| 308 | RAS/P53/ | tctctcatatggatggtggaattactcaatccccaa |
| | PIK3CA | |
| 309 | RAS/P53/ | tagaaaccggtggccaggcacaccagtgtggc |
| | PIK3CA | |
| 310 | RAS/P53/ | MEKMLECAFIVLWLQLGWLSGEDQVTQSPEALRLQEGESSSLNCS |
| | PIK3CA | YTVSGLRGLFWYRQDPGKGPEFLFTLYSAGEEKEKERLKATLTKK |
| | | ESFLHITAPKPEDSATYLCAVQGENSGYSTLTFGKGTMLLVSPDIQ |
| | | NPDPAVYQLRDSKSSDKSVCLFTDFDSQTNVSQSKDSDVYITDKT |
| | | VLDMRSMDFKSNSAVAWSNKSDFACANAFNNSIIPEDTFFPSPESS |
| | | CDVKLVEKSFETDTNLNFQNLSVIGFRILLLKVAGFNLLMTLRLW |
| | | SS |
| 311 | RAS/P53/ | MEKMLECAFIVLWLQLGWLSG |
| | PIK3CA | |
| 312 | RAS/P53/ | MEKMLECAFIVLWLQLGWLSGEDQVTQSPEALRLQEGESSSLNCS |
| | PIK3CA | YTVSGLRGLFWYRQDPGKGPEFLFTLYSAGEEKEKERLKATLTKK |
| | | ESFLHITAPKPEDSATYLCAVQ |
| 313 | RAS/P53/ | VSGLRG |
| | PIK3CA | |
| 314 | RAS/P53/ | LYS |
| | PIK3CA | |
| 315 | RAS/P53/ | CAVQGENSGYSTLTF |
| | PIK3CA | |
| 316 | RAS/P53/ | NSGYSTLTFGKGTMLLVSP |
| | PIK3CA | |
| 317 | RAS/P53/ | DIQNPDPAVYQLRDSKSSDKSVCLFTDFDSQTNVSQSKDSDVYIT |
| | PIK3CA | DKTVLDMRSMDFKSNSAVAWSNKSDFACANAFNNSIIPEDTFFPS |
| | | PESSCDVKLVEKSFETDTNLNFQNLSVIGFRILLLKVAGFNLLMTL |
| | | RLWSS |
| 318 | RAS/P53/ | MGPQLLGYVVLCLLGAGPLEAQVTQNPRYLITVTGKKLTVTCSQ |
| | PIK3CA | NMNHEYMSWYRQDPGLGLRQIYYSMNVEVTDKGDVPEGYKVSR |
| | | KEKRNFPLILESPSPNQTSLYFCASSLGPGLAAYNEQFFGPGTRLTV |
| | | LEDLKNVFPPEVAVFEPSEAEISHTQKATLVCLATGFYPDHVELS |
| | | WWVNGKEVHSGVSTDPQPLKEQPALNDSRYCLSSRLRVSATFWQ |
| | | NPRNHFRCQVQFYGLSENDEWTQDRAKPVTQIVSAEAWGRADC |
| | | GFTSESYQQGVLSATILYEILLGKATLYAVLVSALVLMAMVKRKD |
| | | SRG |
| 319 | RAS/P53/ | MGPQLLGYVVLCLLGAGPLMGPQLLGYVVLCLLGAGPLEAQVTQ |
| | PIK3CA | NPRYLITVTGKKLTVTCSQNMNHEYMSWYRQDPGLGLRQIYYSM |
| | | NVEVTDKGDVPEGYKVSRKEKRNFPLILESPSPNQTSLYFCASSL |
| 320 | RAS/P53/ | MNHEY |
| | PIK3CA | |
| 321 | RAS/P53/ | SMNVEV |
| | PIK3CA | |
| 322 | RAS/P53/ | CASSLGPGLAAYNEQF |
| | PIK3CA | |
| 323 | RAS/P53/ | YNEQFFGPGTRLTVL |
| | PIK3CA | |
| 324 | RAS/P53/ | EDLKNVFPPEVAVFEPSEAEISHTQKATLVCLATGFYPDHVELSW |
| | PIK3CA | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 325 | RAS/P53/ | MKTFAGFSFLFLWLQLDCMSRGEDVEQSLFLSVREGDSSVINCTY |
| | PIK3CA | TDSSSTYLYWYKQEPGAGLQLLTYIFSNMDMKQDQRLTVLLNKK |
| | | DKHLSLRIADTQTGDSAIYFCAEYSSASKIIFGSGTRLSIRPNIQNPD |
| | | PAVYQLRDSKSSDKSVCLFTDFDSQTNVSQSKDSDVYITDKTVLD |
| | | MRSMDFKSNSAVAWSNKSDFACANAFNNSIIPEDTFFPSPESSCDV |
| | | KLVEKSFETDTNLNFQNLSVIGFRILLLKVAGFNLLMTLRLWSS |
| 326 | RAS/P53/ | MKTFAGFSFLFLWLQLDCMSR |
| | PIK3CA | |
| 327 | RAS/P53/ | MKTFAGFSFLFLWLQLDCMSRGEDVEQSLFLSVREGDSSVINCTY |
| | PIK3CA | TDSSSTYLYWYKQEPGAGLQLLTYIFSNMDMKQDQRLTVLLNKK |
| | | DKHLSLRIADTQTGDSAIYFCAE |
| 328 | RAS/P53/ | DSSSTY |
| | PIK3CA | |
| 329 | RAS/P53/ | IFS |
| | PIK3CA | |
| 330 | RAS/P53/ | CAEYSSASKIIF |
| | PIK3CA | |
| 331 | RAS/P53/ | YSSASKIIFGSGTRLSIRP |
| | PIK3CA | |
| 332 | RAS/P53/ | NIQNPDPAVYQLRDSKSSDKSVCLFTDFDSQTNVSQSKDSDVYIT |
| | PIK3CA | DKTVLDMRSMDFKSNSAVAWSNKSDFACANAFNNSIIPEDTFFPS |
| | | PESSCDVKLVEKSFETDTNLNFQNLSVIGFRILLLKVAGFNLLMTL |
| | | RLWSS |
| 333 | RAS/P53/ | MGSWTLCCVSLCILVAKHTDAGVIQSPRHEVTEMGQEVTLRCKPI |
| | PIK3CA | SGHDYLFWYRQTMMRGLELLIYFNNNVPIDDSGMPEDRFSAKMP |
| | | NASFSTLKIQPSEPRDSAVYFCASRANTGELFFGEGSRLTVLEDLK |
| | | NVFPPEVAVFEPSEAEISHTQKATLVCLATGFYPDHVELSWWVNG |
| | | KEVHSGVSTDPQPLKEQPALNDSRYCLSSRLRVSATFWQNPRNHF |
| | | RCQVQFYGLSENDEWTQDRAKPVTQIVSAEAWGRADCGFTSESY |
| | | QQGVLSATILYEILLGKATLYAVLVSALVLMAMVKRKDSRG |
| 334 | RAS/P53/ | MGSWTLCCVSLCILVAKHT |
| | PIK3CA | |
| 335 | RAS/P53/ | MGSWTLCCVSLCILVAKHTDAGVIQSPRHEVTEMGQEVTLRCKPI |
| | PIK3CA | SGHDYLFWYRQTMMRGLELLIYFNNNVPIDDSGMPEDRFSAKMP |
| | | NASFSTLKIQPSEPRDSAVYFCAS |
| 336 | RAS/P53/ | SGHDY |
| | PIK3CA | |
| 337 | RAS/P53/ | FNNNVP |
| | PIK3CA | |
| 338 | RAS/P53/ | CASRANTGELFF |
| | PIK3CA | |
| 339 | RAS/P53/ | NTGELFFGEGSRLTVL |
| | PIK3CA | |
| 340 | RAS/P53/ | EDLKNVFPPEVAVFEPSEAEISHTQKATLVCLATGFYPDHVELSW |
| | PIK3CA | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 341 | RAS/P53/ | MLTASLLRAVIASICVVSSMAQKVTQAQTEISVVEKEDVTLDCVY |
| | PIK3CA | ETRDTTYYLFWYKQPPSGELVFLIRRNSFDEQNEISGRYSWNFQKS |
| | | TSSFNFTITASQVVDSAVYFCALSEGNSGNTPLVFGKGTRLSVIANI |
| | | QNPDPAVYQLRDSKSSDKSVCLFTDFDSQTNVSQSKDSDVYITDK |
| | | TVLDMRSMDFKSNSAVAWSNKSDFACANAFNNSIIPEDTFFPSPES |
| | | SCDVKLVEKSFETDTNLNFQNLSVIGFRILLLKVAGFNLLMTLRL |
| | | WSS |
| 342 | RAS/P53/ | MLTASLLRAVIASICVVSSM |
| | PIK3CA | |
| 343 | RAS/P53/ | MLTASLLRAVIASICVVSSMAQKVTQAQTEISVVEKEDVTLDCVY |
| | PIK3CA | ETRDTTYYLFWYKQPPSGELVFLIRRNSFDEQNEISGRYSWNFQKS |
| | | TSSFNFTITASQVVDSAVYFCALSE |
| 344 | RAS/P53/ | TRDTTYY |
| | PIK3CA | |
| 345 | RAS/P53/ | RNSF |
| | PIK3CA | |
| 346 | RAS/P53/ | CALSEGNSGNTPLVF |
| | PIK3CA | |
| 347 | RAS/P53/ | NSGNTPLVFGKGTRLSVIA |
| | PIK3CA | |
| 348 | RAS/P53/ | NIQNPDPAVYQLRDSKSSDKSVCLFTDFDSQTNVSQSKDSDVYIT |
| | PIK3CA | DKTVLDMRSMDFKSNSAVAWSNKSDFACANAFNNSIIPEDTFFPS |
| | | PESSCDVKLVEKSFETDTNLNFQNLSVIGFRILLLKVAGFNLLMTL |
| | | RLWSS |
| 349 | RAS/P53/ | MGIRLLCRVAFCFLAVGLVDVKVTQSSRYLVKRTGEKVFLECVQ |
| | PIK3CA | DMDHENMFWYRQDPGLGLRLIYFSYDVKMKEKGDIPEGYSVSRE |
| | | KKERFSLILESASTNQTSMYLCASSLSSGSHQETQYFGPGTRLLVL |
| | | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 350 | RAS/P53/ | MGIRLLCRVAFCFLAVGLV |
| | PIK3CA | |
| 351 | RAS/P53/ | MGIRLLCRVAFCFLAVGLVDVKVTQSSRYLVKRTGEKVFLECVQ |
| | PIK3CA | DMDHENMFWYRQDPGLGLRLIYFSYDVKMKEKGDIPEGYSVSRE |
| | | KKERFSLILESASTNQTSMYLCASSL |
| 352 | RAS/P53/ | MDHEN |
| | PIK3CA | |
| 353 | RAS/P53/ | SYDVKM |
| | PIK3CA | |
| 354 | RAS/P53/ | CASSLSSGSHQETQYF |
| | PIK3CA | |
| 355 | RAS/P53/ | QETQYFGPGTRLLVL |
| | PIK3CA | |
| 356 | RAS/P53/ | EDLKNVFPPEVAVFEPSEAEISHTQKATLVCLATGFYPDHVELSW |
| | PIK3CA | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 357 | RAS/P53 | gatatccagaaccctgaccctgccgtgtaccagctgagagactctaaatccagtgacaagtctgtctgcct |
| | | attcaccgattttgattctcaaacaaatgtgtcacaaagtaaggattctgatgtgtatatcacagacaaaact |
| | | gtgctagacatgaggtctatggacttcaagagcaacagtgctgtggcctggagcaacaaatctgactttg |
| | | catgtgcaaacgccttcaacaacagcattattccagaagacaccttcttccccagcccagaaagttcctgt |
| | | gatgtcaagctggtcgagaaaagctttgaaacagatacgaacctaaactttcaaaacctgtcagtgattgg |
| | | gttccgaatcctcctcctgaaagtggccgggtttaatctgctcatgacgctgcggctgtggtccagc |
| 358 | RAS/P53 | atccagaaccctgaccctgccgtgtaccagctgagagactctaaatccagtgacaagtctgtctgcctatt |
| | | caccgattttgattctcaaacaaatgtgtcacaaagtaaggattctgatgtgtatatcacagacaaaactgtg |
| | | ctagacatgaggtctatggacttcaagagcaacagtgctgtggcctggagcaacaaatctgactttgcatg |
| | | tgcaaacgccttcaacaacagcattattccagaagacaccttcttccccagcccagaaagttcctgtgatgt |
| | | caagctggtcgagaaaagctttgaaacagatacgaacctaaactttcaaaacctgtcagtgattgggttcc |
| | | gaatcctcctcctgaaagtggccgggtttaatctgctcatgacgctgcggctgtggtccagc |
| 359 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gtgcgtgctggacatgcggagcatggacttcaagagcaactccgccgtggcctggtccaacaagtccg |
| | | atttcgcctgcgccaacgccttcaacaacagcattatccccgaggacacattcttcccaagcagcgacgt |
| | | gccctgcgacgtgaagctggtggaaaagagcttcgagacagacaccaacctgaacttccagaacctga |
| | | gcgtgatcggcttcagaatcctgctgctgaaagtggccggctttaacctgctgatgaccctgcggctgtg |
| | | gtccagc |
| 360 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcgacgtgaagctggtggaaaagagcttcgagacagacaccaacctgaacttccagaacctga |
| | | gcgtgatcggcttcagaatcctgctgctgaaagtggccggctttaacctgctgatgaccctgcggctgtg |
| | | gtccagc |
| 361 | RAS/P53 | cctgatcctgccgtgtaccagctgcgggatagcaagagcagcgacaagagcgtgtgcctgttcaccga |
| | | cttcgacagccagaccaacgtgtctcagtctaaggatagtgatgtgtatatcaccgacaagaccgtgctg |
| | | gacatgcggagcatggacttcaagagcaacagcgccgtggcctggtccaacaagagcgacttcgcctg |
| | | cgccaacgccttcaacaacagcatcatccccgaggacaccttcttccccagccccgagagcagctgcg |
| | | acgtgaaactggtggagaagagcttcgagacagacaccaacctgaacttccagaacctgagcgtgatc |
| | | ggcttcagaatcctgctgctgaaggtggccggcttcaacctgctgatgaccctgcggctgtggagcagc |
| 362 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gtgcgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcgacgtgaagctggtggaaaagagcttcgagacagacaccaacctgaacttccagaacctga |
| | | gcgtgatcggcttcagaatcctgctgctgaaagtggccggctttaacctgctgatgaccctgcggctgtg |
| | | gtccagc |
| 363 | RAS/P53 | cctgatcctgccgtgtaccagctggggatagcaagagcagcgacaagagcgtgtgtctgttcaccgac |
| | | ttcgacagccagaccaacgtgtcccagagcaaggactccgacgtgtacatcaccgataagtgcgtgctg |
| | | gacatgcggagcatggacttcaagagcaactccgccgtggcctggtccaacaagtccgatttcgcctgc |
| | | gccaacgccttcaacaacagcattatccccgaggacacattcttcccaagccccgagagcagctgcgac |
| | | gtgaagctggtggaaaagagcttcgagacagacaccaacctgaacttccagaacctgagcgtgatcgg |
| | | cttcagaatcctgctgctgaaagtggccggctttaacctgctgatgaccctgcggctgtggtccagc |
| 364 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagcagcgacgt |
| | | gccctgcgacgtgaagctggtggaaaagagcttcgagacagacaccaacctgaacttccagaacctga |
| | | gcgtgatcggcttcagaatcctgctgctgaaagtggccggctttaacctgctgatgaccctgcggctgtg |
| | | gtccagc |
| 365 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaaccgcgccgtggcctggtccaacaagtccg |
| | | atttcgcctgcgccaacgccttcaacaacagcattatccccgaggacacattcttcccaagccccgagag |
| | | cagctgcgacgtgaagctggtggaaaagagcttcgagacagacaccaacctgaacttccagaacctga |
| | | gcgtgatcggcttcagaatcctgctgctgaaagtggccggctttaacctgctgatgaccctgcggctgtg |
| | | gtccagc |
| 366 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaaccgcgccgtggcctggtccaacaagtccg |
| | | atttcgcctgcgccaacgccttcaacaacagcattatccccgaggacacattcttcccaagcagcgacgt |
| | | gccctgcgacgtgaagctggtggaaaagagcttcgagacagacaccaacctgaacttccagaacctga |
| | | gcgtgatcggcttcagaatcctgctgctgaaagtggccggctttaacctgctgatgaccctgcggctgtg |
| | | gtccagc |
| 367 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagcagcgacag |
| | | cagctgcgacgtgaagctggtggaaaagagcttcgagacagacaccaacctgaacttccagaacctga |
| | | gcgtgatcggcttcagaatcctgctgctgaaagtggccggctttaacctgctgatgaccctgcggctgtg |
| | | gtccagc |
| 368 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcggtacccagagttttgggttgttggaccctaaattgtgctatctgttggacggaatcctgttcatt |
| | | tacggagtgatcctgaccgcattgttcctgcgggttaagttttccagatccgcagacgcacccgcttaccaa |
| | | caagggcagaaccaactctacaatgaacttaatttggggcggagggaagagtacgacgttctggacaaa |
| | | aggaggggacgagatcccgagatgggcggaaagcctcagcgaaggaagaatccgcaagaggggct |
| | | ttataacgaactccagaaagataaaatggcagaagcctatagtgagattgggatgaagggagaaagga |
| | | ggaggggtaaaggtcacgatgggttgtaccaaggattgagcacagccactaaagatacctatgatgcgc |
| | | tccacatgcaggccctgccgccaaga |
| 369 | RAS/P53 | atccagaaccctgaacctgccgtgtaccagctgaaggaccccagaagccaggacagcaccctgtgcct |
| | | gttcaccgacttcgacagccagatcaacgtgcccaagacaatggaaagcggcaccttcatcaccgacaa |
| | | gaccgtgctggacatgaaggctatggacagcaagagcaacggcgccattgcctggtccaaccagacc |
| | | agcttcacatgccaggacatcttcaaagagacaaacgccacctacccctccagcgacgtgccctgtgac |
| | | gccaccctgaccgagaagtccttcgagacagacatgaacctcaacttccagaacctgagcgtgatgggc |
| | | ctgcgaatcctgctgctgaaagtggccggcttcaacctgctgatgaccctgcggctgtggtccagc |
| 370 | RAS/P53 | gtgcaggaccctgaccctagcgtgtaccagatgagaagccccaagaccggcaggaccgtgtgcctgtt |
| | | caccgacttcgacagccaggccgacttccagaagcctgagggggccaccgtgatctccctgaacagca |
| | | ccgtgctggacatgaaagtgatggacagcaagagcaacggcgctctgacctggaacagcgagacaaa |
| | | catcgagtgcaaagagaacttccagcagcccttctaccccagcagcaactactcctgcgacgccaagct |
| | | gatcgagaagtccttcgagacagacatggacctgaacttctacaacctgctggtcatcggcctgagaatc |
| | | ctgctgctgaaagtgatcggcttcaacctgttcatgaccctgaggctgtggtcctgc |
| 371 | RAS/P53 | gaggacctgaacaaggtgttcccacccgaggtcgctgtgtttgagccatcagaagcagagatctcccac |
| | | acccaaaaggccacactggtgtgcctggccacaggcttcttccccgaccacgtggagctgagctggtg |
| | | ggtgaatgggaaggaggtgcacagtggggtcagcacagacccgcagcccctcaaggagcagcccgc |
| | | cctcaatgactccagatactgcctgagcagccgcctgagggtctcggccaccttctggcagaacccccg |
| | | caaccacttccgctgtcaagtccagttctacgggctctcggagaatgacgagtggacccaggatagggc |
| | | caaacccgtcacccagatcgtcagcgccgaggcctggggtagagcagactgtggctttacctcggtgtc |
| | | ctaccagcaaggggtcctgtctgccaccatcctctatgagatcctgctagggaaggccaccctgtatgct |
| | | gtgctggtcagcgcccttgtgttgatggccatggtcaagagaaaggatttc |
| 372 | RAS/P53 | gaggacctgaaaaacgtgttcccacccgaggtcgctgtgtttgagccatcagaagcagagatctcccac |
| | | acccaaaaggccacactggtatgcctggccacaggcttctaccccgaccacgtggagctgagctggtg |
| | | ggtgaatgggaaggaggtgcacagtggggtcagcacagacccgcagcccctcaaggagcagcccgc |
| | | cctcaatgactccagatactgcctgagcagccgcctgagggtctcggccaccttctggcagaacccccg |
| | | caaccacttccgctgtcaagtccagttctacgggctctcggagaatgacgagtggacccaggatagggc |
| | | caaacccgtcacccagatcgtcagcgccgaggcctggggtagagcagactgtggcttcacctccgagt |
| | | cttaccagcaaggggtcctgtctgccaccatcctctatgagatcttgctagggaaggccaccttgtatgcc |
| | | gtgctggtcagtgccctcgtgctgatggccatggtcaagagaaaggattccagaggc |
| 373 | RAS/P53 | gaggacctgaaaaacgtgttcccacccgaggtcgctgtgtttgagccatcagaagcagagatctcccac |
| | | acccaaaaggccacactggtatgcctggccacaggcttctaccccgaccacgtggagctgagctggtg |
| | | ggtgaatgggaaggaggtgcacagtggggtcagcacagacccgcagcccctcaaggagcagcccgc |
| | | cctcaatgactccagatactgcctgagcagccgcctgagggtctcggccaccttctggcagaacccccg |
| | | caaccacttccgctgtcaagtccagttctacgggctctcggagaatgacgagtggacccaggatagggc |
| | | caaacccgtcacccagatcgtcagcgccgaggcctggggtagagcagactgtggcttcacctccgagt |
| | | cttaccagcaaggggtcctgtctgccaccatcctctatgagatcttgctagggaaggccaccttgtatgcc |
| | | gtgctggtcagtgccctcgtgctgatggccatggtcaagagaaaggattccagaggc |
| 374 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtgtaccgacccccagccactgaaagaacagcccgcc |
| | | ctgaacgacagccggtactgcctgagcagcagactgagagtgtccgccaccttctggcagaacccccg |
| | | gaaccacttcagatgccaggtgcagttctacggcctgagcgagaacgacgagtggacccaggacaga |
| | | gccaagcccgtgacccagatcgtgtctgccgaagcctggggcagagccgattgcggctttaccagcga |
| | | gagctaccagcagggcgtgctgagcgccaccatcctgtacgagatcctgctgggcaaggccaccctgt |
| | | acgccgtgctggtgtctgccctggtgctgatggctatggtcaagcggaaggacagccgggga |
| 375 | RAS/P53 | gaggacctgaagaacgtgttcccacctgaggtggccgtgttcgagcctagcaaggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtgtaccgacccccagccactgaaagaacagcccgcc |
| | | ctgaacgacagccggtactgcctgagcagcagactgagagtgtccgccaccttctggcagaacccccg |
| | | gaaccacttcagatgccaggtgcagttctacggcctgagcgagaacgacgagtggacccaggacaga |
| | | gccaagcccgtgacccagatcgtgtctgccgaagcctggggcagagccgattgcggcatcaccagcg |
| | | ccagctaccaccagggcgtgctgagcgccaccatcctgtacgagatcctgctgggcaaggccaccctg |
| | | tacgccgtgctggtgtctgccctggtgctgatggctatggtcaagcggaaggacagccgggga |
| 376 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 377 | RAS/P53 | Gacc(gg)tgaagaacgtgttcccacctgaggtggccgtgttcgagcctagcgaggccgagatcagcc |
| | | acacccagaaagccaccctcgtgtgcctggctaccggcttctaccccgaccatgtggaactgtcttggtg |
| | | ggtcaacggcaaagaggtgcacagcggcgtgtctaccgacccccagccactgaaagaacagcccgc |
| | | cctgaacgacagccggtactgcctgagcagcagactgagagtgtccgccaccttctggcagaaccccc |
| | | ggaaccacttcagatgccaggtgcagttctacggcctgagcgagaacgacgagtggacccaggacag |
| | | agccaagcccgtgacccagatcgtgtctgccgaagcctggggcagagccgattgcggctttaccagcg |
| | | agagctaccagcagggcgtgctgagcgccaccatcctgtacgagatcctgctgggcaaggccaccctg |
| | | tacgccgtgctggtgtctgccctggtgctgatggctatggtcaagcggaaggacagccgggga |
| 378 | RAS/P53 | gaggacctgaagaacgtgttcccacctgaggtggccgtgttcgagcccagcgaggccgagatttcaca |
| | | cactcagaaagccacactcgtatgtctggcgactggtttctttcccgaccacgtggagctgtcttggtgggt |
| | | gaacggcaaagaggtgcacagcggggtctctaccgacccccagccactgaaagagcagcccgccct |
| | | gaacgacagccggtactgcctctcttctcggctgagagtgtccgccaccttctggcagaacccccggaa |
| | | ccacttcagatgccaggtgcagttctacggcctgagcgagaacgacgagtggacccaggacagagcc |
| | | aagcctgtgacccagatcgtgagtgctgaagcgtggggacgcgccgattgcggctttaccagcgagag |
| | | ctaccagcagggcgtgctgtctgccaccatcctgtacgagatcctgctgggcaaggccaccctgtacgc |
| | | cgtgctggtgtccgccctggtgctgatggctatggtgaagcggaaggacttc |
| 379 | RAS/P53 | gaggacctgaacaaggtgttcccacctgaggtggccgtgttcgagcccagcgaggccgagatttcaca |
| | | cactcagaaagccacactcgtatgtctggcgactggtttctttcccgaccacgtggagctgtcttggtgggt |
| | | gaacggcaaagaggtgcacagcggggtctctaccgacccccagccactgaaagagcagcccgccct |
| | | gaacgacagccggtactgcctctcttctcggctgagagtgtccgccaccttctggcagaacccccggaa |
| | | ccacttcagatgccaggtgcagttctacggcctgagcgagaacgacgagtggacccaggacagagcc |
| | | aagcctgtgacccagatcgtgagtgctgaagcgtggggacgcgccgattgcggctttaccagcgtgag |
| | | ctaccagcagggcgtgctgtctgccaccatcctgtacgagatcctgctgggcaaggccaccctgtacgc |
| | | cgtgctggtgtccgccctggtgctgatggctatggtgaagcggaaggacttc |
| 380 | RAS/P53 | gaggacctgaagaacgtgttcccacctgaggtggccgtgttcgagcctagcaaggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggcatcaccagcgc |
| | | cagctaccaccagggcgtgctgagcgccaccatcctgtacgagatcctgctgggcaaggccaccctgt |
| | | acgccgtgctggtgtctgccctggtgctgatggctatggtcaagcggaaggacagccgggga |
| 381 | RAS/P53 | gaggacctgaagaacgtgttcccacctgaggtggccgtgttcgagcctagcaaggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 382 | RAS/P53 | gaggacctgaagaacgtgttcccacctgaggtggccgtgttcgagcctagcaaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggcatcaccagcga |
| | | gagctaccagcagggcgtgctgagcgccaccatcctgtacgagatcctgctgggcaaggccaccctgt |
| | | acgccgtgctggtgtctgccctggtgctgatggctatggtcaagcggaaggacagccgggga |
| 383 | RAS/P53 | gaggacctgaagaacgtgttcccacctgaggtggccgtgttcgagcctagcaaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgcc |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 384 | RAS/P53 | gaggacctgaagaacgtgttcccacctgaggtggccgtgttcgagcctagcaaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccaccagggcgtgctgagcgccaccatcctgtacgagatcctgctgggcaaggccaccctgta |
| | | cgccgtgctggtgtctgccctggtgctgatggctatggtcaagcggaaggacagccgggga |
| 385 | RAS/P53 | gaggacctgaagaacgtgttcccacctgaggtggccgtgttcgagcctagcagggccgagatcgccca |
| | | cacccagaaagccaccctcgtgtgcctggctaccggcttctacccgaccatgtggaactgtcttggtggg |
| | | tcaacggcaaagaggtgcacagcggcgtgtctaccgacccccagccactgaaagaacagcccgccct |
| | | gaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 386 | RAS/P53 | gaggacctgaagaacgtgttcccacctgaggtggccgtgttcgagcctagcagggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 387 | RAS/P53 | gaggacctgaagaacgtgttcccacctgaggtggccgtgttcgagcctagcaaggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcggactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggcatcaccagcgc |
| | | cagctaccaccagggcgtgctgagcgccaccatcctgtacgagatcctgctgggcaaggccaccctgt |
| | | acgccgtgctggtgtctgccctggtgctgatggctatggtcaagcggaaggacagccgggga |
| 388 | RAS/P53 | gaggacctgaagaacgtgttcccacctgaggtggccgtgttcgagcctagcaaggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcggactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 389 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcggactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 390 | RAS/P53 | gaggatctgcggaacgtgaccccacccaaggtgtccctgttcgagcccagcaaggccgagatcgcca |
| | | ataaacagaaggccaccctcgtgtgcctggccagaggcttcttccccgaccacgtggaactgtcttggtg |
| | | ggtcaacggcaaagaggtgcacagcggcgtcagcaccgaccctcaggcctacaaagagagcaacta |
| | | cagctactgcctgagcagtcggctgcgggtgtccgccaccttctggcacaacccccggaaccacttcag |
| | | atgccaggtgcagttccacggcctgagcgaagaggacaagtggcccgagggcagccccaagcctgtc |
| | | acccagaacatcagcgccgaggcctggggcagagcctgtggcatcaccagcagctaccagcagggc |
| | | gtgctgagcgccaccatcctgtacgagatcctgctgggcaaggccaccctgtacgccgtgctggtgtcc |
| | | accctggtggtcatggctatggtcaagcggaagaacagc |
| 391 | RAS/P53 | gaggacctccagaaagtgaccccccctaccgtgaccgtgttcgagccttctgaggccgagatcagccg |
| | | gacccagaaagccacactcgtgtgcctggctcggggcttctaccccgatcacgtggaactgtcttggtgg |
| | | gtcaacggcaaagaagtgaccagcggcttcagcaccgacccccagcctgacaaagagcggcccagc |
| | | gagaacgacagcagctactgcctgagcagcagactgagagtgtccgccagcttctggcacaacccccg |
| | | gaaccacttcagatgccaggtgcagttctacggcctgggcgacgacgacgagtggaagtacgacaga |
| | | gtgaagcccatcacccagaacatcagcgccgaagcctggggcagagccgattgcggctttacctccgt |
| | | cagctaccaccagggcgtgctgagcgccaccatcctgtacgagatcctgctgggcaaggccaccctgt |
| | | acgccgtgctggtgtctatcctggtgctgatggctaaagtgaagcggaagggcagc |
| 392 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggatcccagagcttc |
| | | ggcctgctggaccccaagctgtgctacctgctggatggcatcctgttcatctacggcgtgatcctgaccg |
| | | ccctgttcctgagagtgaagttcagcagaagcgccgacgcccctgcctaccagcagggacagaaccaa |
| | | ctgtacaacgagctgaacctgggcagacgggaagagtacgacgtgctggacaagcggagaggcagg |
| | | gaccctgagatgggcggaaagccccagcggagaaagaacccccaggaaggactgtataacgaactc |
| | | cagaaagacaagatggccgaagcctacagcgagatcggcatgaagggcgagcggagaagaggcaa |
| | | gggccacgatggcctgtaccagggcctgagcaccgccaccaaggacacctatgacgccctgcacatg |
| | | caggccctgcctccaaga |
| 393 | RAS/P53 | gggtgtccagccctacccacaggtgtgggcggcacaccctttccttctctggccccaccaatcatgctgc |
| | | tggtggatggaaagcagcagatggtggtggtctgcctggtccttgatgttgcaccccctggccttgacag |
| | | ccccatctggttctcagccggcaatggcagtgcactggatgccttcacctatggcccttccccagcaacg |
| | | gatggcacctggaccaacttggcccatctctccctgccttctgaggagctggcatcctgggagcctttggt |
| | | ctgccacactgggcctggggctgagggtcacagcaggagtacacagcccatgcatctgtcaggagag |
| | | gcttctacagccaggacctgcccccaggagcctctcagggggacaccgggtggggcgctgtggctgg |
| | | gggtcctgcggctgctgctcttcaagctgctgctgtttgacctgctcctgacctgcagctgcctgtgcgac |
| | | cccgcgggcccgctgccttcccccgcaaccaccacccgcctgcgagccctcggntcccatcgactgca |
| | | cccggccacggagactgggggacgagaggccaccagctcacccagaccccagcctcgggaccgcc |
| | | gctggggtgacacccctccgggtcggaagcccgggagcccagtatggggggaagggtcttacctcag |
| | | cagttaccccacttgcccagcacaggcctggtgctcaagatctcgcctcagggctccttcctccagtcttg |
| | | gagcattttttgcaggtgacctgcctcctcctctgcaggctggagctgcc |
| 394 | RAS/P53 | accccattcccttctctggcccctcccatcatgctgctggtggacggcaagcagcagatggtggtcgtgt |
| | | gcctggtgctggatgtggccccacctggactggacagccccatctggtttagcgccggcaatggctctg |
| | | ccctggacgcctttacctacggcccaagccctgccaccgatggcacctggacaaatctggcccacctga |
| | | gcctgcccagcgaggaactggcttcttgggagcctctcgtgtgccacacaggacctggcgctgaggga |
| | | cacagcagatccacccagcctatgcacctgtctggcgaggccagcaccgccagaacctgtcctcagga |
| | | acctctgagaggcacccctggcggagcactgtggctgggagtgctgagactgctgctgttcaagctgct |
| | | gctgtttgacctgctgctgacctgctcctgtctgtgcgatcctgccggccctctgccttctcctgccaccac |
| | | cacaagactgagagccctgggcagccacagactgcaccctgccacagagacaggcggcagagagg |
| | | ccacaagcagccctagaccccagcccagagacagaagatggggcgataccccccctggcagaaagc |
| | | ctggatctccagtgtggggcgagggcagctacctgagcagctaccctacatgccctgcccaggcttggt |
| | | gcagcagaagcgcactgagagcacccagctctagcctgggagccttctttgccggcgatctgcctccac |
| | | cactccaggctggcgctgct |
| 395 | RAS/P53 | accccattcccttctctggcccctcccatcatgctgctggtggacggcaagcagcagatggtggtcgtgt |
| | | gcctggtgctggatgtggccccacctggactggacagccccatctggtttagcgccggcaatggctctg |
| | | ccctggacgcctttacctacggcccaagccctgccaccgatggcacctggacaaatctggcccacctga |
| | | gcctgcccagcgaggaactggcttcttgggagcctctcgtgtgccacacaggacctggcgctgaggga |
| | | cacagcagatccacccagcctatgcacctgtctggcgaggccagcaccgccagaacctgtcctcagga |
| | | acctctgagaggcacccctggcggagcactgtggctgggagtgctgagactgctgctgttcaagctgct |
| | | gctgtttgacctgctgctgacctgctcctgtctgtgcgatcctgccggccctctgccttctcctgccaccac |
| | | cacaagactgagagccctgggcagccacagactgcaccctgccacagagacaggggcagagagg |
| | | ccacaagcagccctagaccccagcccagagacagaagatggggcgataccccccctggcagaaagc |
| | | ctggatctccagtc |
| 396 | RAS/P53 | gataaacaacttgatgcagatgtttcccccaagcccactatttttcttccttcaattgctgaaacaaaactcc |
| | | agaaggctggaacatacctttgtcttcttgagaaatttttcccagatattattaagatacattggcaagaaaa |
| | | gaagagcaacacgattctgggatcccaggaggggaacaccatgaagactaacgacacatacatgaaattt |
| | | agctggttaacggtgccagaagagtcactggacaaagaacacagatgtatcgtcagacatgagaataat |
| | | aaaaacggaattgatcaagaaattatctttcctccaataaagacagatgtcaccacagtggatcccaaata |
| | | caattattcaaaggatgcaaatgatgtcatcacaatggatcccaaagacaattggtcaaaagatgcaaatg |
| | | atacactactgctgcagctcacaaacacctctgcatattacatgtacctcctcctgctcctcaagagtgtggt |
| | | ctattttgccatcatcacctgctgtctgcttggaagaacggctttctgctgcaatggagagaaatca |
| 397 | RAS/P53 | gacaagcagctggacgccgacgtgtcccccaagcctaccatcttcctgccctctatcgccgagacaaag |
| | | ctccagaaggccggcacctacctgtgcctgctggaaaagttcttcccagatgtgattaagatccactggc |
| | | aggaaaagaagtccaacaccatcctgggcagccaggaaggcaacaccatgaagaccaacgacaccta |
| | | catgaagttcagctggctgaccgtgcccgagaagtccctggacaaagaacacagatgcatcgtgcggc |
| | | acgaaaacaacaagaacggcgtggaccaggaaatcatcttcccacccatcaagaccgatgtgattacaa |
| | | tggaccccaaggacaactgctccaaggacgccaacgataccctgctgctccagctgaccaacaccagc |
| | | gcctactacatgtacttgttgctgctgctgaagtccgtggtgtacttcgccatcatcacatgctgcctgctgc |
| | | ggagaaccgccttctgctgcaacggcgagaagtct |
| 398 | RAS/P53 | agtcagcctcataccaaaccatccgtttttgtcatgaaaaatggaacaaatgtcgcttgtctggtgaaggaa |
| | | ttctaccccaaggatataagaataaatctcgtgtcatccaagaagataacagagtttgatcctgctattgtca |
| | | tctctcccagtgggaagtacaatgctgtcaagcttggtaaatatgaagattcaaattcagtgacatgttcagt |
| | | tcaacacgacaataaaactgtgcactccactgactttgaagtgaagacagattctacagatcacgtaaaac |
| | | caaaggaaactgaaaacacaaagcaaccttcaaagagctgccataaacccaaagccatagttcataccg |
| | | agaaggtgaacatgatgtccctcacagtgcttgggctacgaatgctgtttgcaaagactgttgccgtcaatt |
| | | ttctcttgactgccaagttatttttcttg |
| 399 | RAS/P53 | agccagcctcacaccaagcccagcgtgttcgtgatgaagaacggcaccaacgtggcctgcctcgtgaa |
| | | agagttctaccccaaggacatcaggatcaacctggtgtccagcaagaagatcaccgagttcgaccccgc |
| | | catcgtgatctcccccagcggcaagtacaacgccgtgaagctggggaagtacgaggacagcaacagc |
| | | gtgacctgctccgtgcagcacgacaacaagaccgtgcacagcaccgactttgaagtgaaaaccgactc |
| | | caccgaccacgtgaagcccaaagagacagagaacaccaagcagcccagcaagagctgccacaagc |
| | | ccaaggccatcgtgcacaccgagaaagtgaacatgatgagcctgaccgtgctgggcctgcggatgctg |
| | | ttcgccaaaaccgtggccgtgaacttcctgctgaccgccaagctgttcttcctc |
| 400 | RAS/P53 | atccagaaccctgatcctgccgtgtacatcaccgataagaccgtgctggacatgcggagcatggacttca |
| | | agagcaactccgccgtggcctggtccaacaagtccgatttcgcctgcgccaacgccttcaacaacagca |
| | | ttatccccgaggacacattcttcccaagccccgagagcagctgcgacgtgaagctggtggaaaagagct |
| | | tcgagacagacaccaacctgaacttccagaacctgagcgtgatcggcttcagaatcctgctgctgaaagt |
| | | ggccggctttaacctgctgatgaccctgcggctgtggtccagc |
| 401 | RAS/P53 | gaggacctgaagaacgtgttcccacctgaggtggccgtgttcgagcctagcagggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtgtaccgacccccagccactgaaagaacagcccgcc |
| | | ctgaacgacagccggtactgcctgagcagcagactgagagtgtccgccaccttctggcagaacccccg |
| | | gaaccacttcagatgccaggtgcagttctacggcctgagcgagaacgacgagtggacccaggacaga |
| | | gccaagcccgtgacccagatcgtgtctgccgaagcctggggcagagccgattgcggctttaccagcgg |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 402 | RAS/P53 | gaggacctgaagaacgtgttcccacctgaggtggccgtgttcgagcctagcagggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtgtaccgacccccagccactgaaagaacagcccgcc |
| | | ctgaacgacagccggtactgcctgagcagcagactgagagtgtccgccaccttctggcagaacccccg |
| | | gaaccacttcagatgccaggtgcagttctacggcctgagcgagaacgacgagtggacccaggacaga |
| | | gccaagcccgtgacccagatcgtgtctgccgaagcctggggcagagccgattgcggctttaccagcga |
| | | gagctaccagcagggcgtgctgagcgccaccatcctgtacgagatcctgctgggcaaggccaccctgt |
| | | acgccgtgctggtgtctgccctggtgctgatggctatggtcaagcggaaggacagccgggga |
| 403 | RAS/P53 | DIQNPDPAVYQLRDSKSSDKSVCLFTDFDSQTNVSQSKDSDVYIT |
| | | DKTVLDMRSMDFKSNSAVAWSNKSDFACANAFNNSIIPEDTFFPS |
| | | PESSCDVKLVEKSFETDTNLNFQNLSVIGFRILLLKVAGFNLLMTL |
| | | RLWSS |
| 404 | RAS/P53 | IQNPDPAVYQLRDSKSSDKSVCLFTDFDSQTNVSQSKDSDVYITD |
| | | KCVLDMRSMDFKSNSAVAWSNKSDFACANAFNNSIIPEDTFFPSS |
| | | DVPCDVKLVEKSFETDTNLNFQNLSVIGFRILLLKVAGFNLLMTLR |
| | | LWSS |
| 405 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 406 | RAS/P53 | PDPAVYQLRDSKSSDKSVCLFTDFDSQTNVSQSKDSDVYITDKTV |
| | | LDMRSMDFKSNSAVAWSNKSDFACANAFNNSIIPEDTFFPSPESSC |
| | | DVKLVEKSFETDTNLNFQNLSVIGFRILLLKVAGFNLLMTLRLWSS |
| 407 | RAS/P53 | IQNPDPAVYQLRDSKSSDKSVCLFTDFDSQTNVSQSKDSDVYITD |
| | | KCVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 408 | RAS/P53 | PDPAVYQLRDSKSSDKSVCLFTDFDSQTNVSQSKDSDVYITDKCV |
| | | LDMRSMDFKSNSAVAWSNKSDFACANAFNNSIIPEDTFFPSPESSC |
| | | DVKLVEKSFETDTNLNFQNLSVIGFRILLLKVAGFNLLMTLRLWSS |
| 409 | RAS/P53 | IQNPDPAVYQLRDSKSSDKSVCLFTDFDSQTNVSQSKDSDVYITD |
| | | KTVLDMRSMDFKSNRAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 410 | RAS/P53 | IQNPDPAVYQLRDSKSSDKSVCLFTDFDSQTNVSQSKDSDVYITD |
| | | KTVLDMRSMDFKSNRAVAWSNKSDFACANAFNNSIIPEDTFFPSS |
| | | DVPCDVKLVEKSFETDTNLNFQNLSVIGFRILLLKVAGFNLLMTLR |
| | | LWSS |
| 411 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSS |
| | | DSSCDVKLVEKSFETDTNLNFQNLSVIGFRILLLKVAGFNLLMTLR |
| | | LWSS |
| 412 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCGTQSFGLLDPKLCYLLDGILFIYGVILTALFLRVKFSRSADAP |
| | | AYQQGQNQLYNELNLGRREEYDVLDKRRGRDPEMGGKPQRRKN |
| | | PQEGLYNELQKDKMAEAYSEIGMKGERRRGKGHDGLYQGLSTA |
| | | TKDTYDALHMQALPPR |
| 413 | RAS/P53 | IQNPEPAVYQLKDPRSQDSTLCLFTDFDSQINVPKTMESGTFITDK |
| | | TVLDMKAMDSKSNGAIAWSNQTSFTCQDIFKETNATYPSSDVPCD |
| | | ATLTEKSFETDMNLNFQNLSVMGLRILLLKVAGFNLLMTLRLWSS |
| 414 | RAS/P53 | VQDPDPSVYQMRSPKTGRTVCLFTDFDSQADFQKPEGATVISLNS |
| | | TVLDMKVMDSKSNGALTWNSETNIECKENFQQPFYPSSNYSCDA |
| | | KLIEKSFETDMDLNFYNLLVIGLRILLLKVIGFNLFMTLRLWSC |
| 415 | RAS/P53 | EDLNKVFPPEVAVFEPSEAEISHTQKATLVCLATGFFPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSVSYQQGVLSATILYEILLGKATLYAVLVSALVLMAMVKRKDF |
| 416 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 417 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 418 | RAS/P53 | EDLKNVFPPEVAVFEPSEAEISHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 419 | RAS/P53 | EDLKNVFPPEVAVFEPSKAEIAHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDSR |
| | | G |
| 420 | RAS/P53 | DLKNVFPPEVAVFEPSEAEISHTQKATLVCLATGFYPDHVELSWW |
| | | VNGKEVHSGVSTDPQPLKEQPALNDSRYCLSSRLRVSATFWQNPR |
| | | NHFRCQVQFYGLSENDEWTQDRAKPVTQIVSAEAWGRADCGFTS |
| | | ESYQQGVLSATILYEILLGKATLYAVLVSALVLMAMVKRKDSRG |
| 421 | RAS/P53 | EDLKNVFPPEVAVFEPSEAEISHTQKATLVCLATGFFPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDF |
| 422 | RAS/P53 | EDLNKVFPPEVAVFEPSEAEISHTQKATLVCLATGFFPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSVSYQQGVLSATILYEILLGKATLYAVLVSALVLMAMVKRKDF |
| 423 | RAS/P53 | EDLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDSR |
| | | G |
| 424 | RAS/P53 | EDLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 425 | RAS/P53 | EDLKNVFPPEVAVFEPSKAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSESYQQGVLSATILYEILLGKATLYAVLVSALVLMAMVKRKDSR |
| | | G |
| 426 | RAS/P53 | EDLKNVFPPEVAVFEPSKAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSASYQQGVLSATILYEILLGKATLYAVLVSALVLMAMVKRKDSR |
| | | G |
| 427 | RAS/P53 | EDLKNVFPPEVAVFEPSKAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYHQGVLSATILYEILLGKATLYAVLVSALVLMAMVKRKDSR |
| | | G |
| 428 | RAS/P53 | EDLKNVFPPEVAVFEPSRAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 429 | RAS/P53 | EDLKNVFPPEVAVFEPSRAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 430 | RAS/P53 | EDLKNVFPPEVAVFEPSKAEIAHTQKATLVCLATGFYPDHVELSW |
| | | WVNGKEVHSGVSTDPQPLKEQPALNDSRYCLSSGLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDSR |
| | | G |
| 431 | RAS/P53 | EDLKNVFPPEVAVFEPSKAEIAHTQKATLVCLATGFYPDHVELSW |
| | | WVNGKEVHSGVSTDPQPLKEQPALNDSRYCLSSGLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 432 | RAS/P53 | EDLKNVFPPEVAVFEPSEAEISHTQKATLVCLATGFYPDHVELSW |
| | | WVNGKEVHSGVSTDPQPLKEQPALNDSRYCLSSGLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 433 | RAS/P53 | EDLRNVTPPKVSLFEPSKAEIANKQKATLVCLARGFFPDHVELSW |
| | | WVNGKEVHSGVSTDPQAYKESNYSYCLSSRLRVSATFWHNPRNH |
| | | FRCQVQFHGLSEEDKWPEGSPKPVTQNISAEAWGRACGITSSYQQ |
| | | GVLSATILYEILLGKATLYAVLVSTLVVMAMVKRKNS |
| 434 | RAS/P53 | EDLQKVTPPTVTVFEPSEAEISRTQKATLVCLARGFYPDHVELSW |
| | | WVNGKEVTSGFSTDPQPDKERPSENDSSYCLSSRLRVSASFWHNP |
| | | RNHFRCQVQFYGLGDDDEWKYDRVKPITQNISAEAWGRADCGFT |
| | | SVSYHQGVLSATILYEILLGKATLYAVLVSILVLMAKVKRKGS |
| 435 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLRVKFSRSADAPAYQQG |
| | | QNQLYNELNLGRREEYDVLDKRRGRDPEMGGKPQRRKNPQEGL |
| | | YNELQKDKMAEAYSEIGMKGERRRGKGHDGLYQGLSTATKDTY |
| | | DALHMQALPPR |
| 436 | RAS/P53 | GCPALPTGVGGTPFPSLAPPIMLLVDGKQQMVVVCLVLDVAPPGL |
| | | DSPIWFSAGNGSALDAFTYGPSPATDGTWTNLAHLSLPSEELASW |
| | | EPLVCHTGPGAEGHSRSTQPMHLSGEASTARTCPQEPLRGTPGGA |
| | | LWLGVLRLLLFKLLLFDLLLTCSCLCDPAGPLPSPATTTRLRALG |
| | | SHRLHPATETGGREATSSPRPQPRDRRWGDTPPGRKPGSPVWGEG |
| | | SYLSSYPTCPAQAWCSRSRLRAPSSSLGAFFAGDLPPPLQAGAA |
| 437 | RAS/P53 | TPFPSLAPPIMLLVDGKQQMVVVCLVLDVAPPGLDSPIWFSAGNG |
| | | SALDAFTYGPSPATDGTWTNLAHLSLPSEELASWEPLVCHTGPGA |
| | | EGHSRSTQPMHLSGEASTARTCPQEPLRGTPGGALWLGVLRLLLF |
| | | KLLLFDLLLTCSCLCDPAGPLPSPATTTRLRALGSHRLHPATETG |
| | | GREATSSPRPQPRDRRWGDTPPGRKPGSPVWGEGSYLSSYPTCPA |
| | | QAWCSRSALRAPSSSLGAFFAGDLPPPLQAGAA |
| 438 | RAS/P53 | TPFPSLAPPIMLLVDGKQQMVVVCLVLDVAPPGLDSPIWFSAGNG |
| | | SALDAFTYGPSPATDGTWTNLAHLSLPSEELASWEPLVCHTGPGA |
| | | EGHSRSTQPMHLSGEASTARTCPQEPLRGTPGGALWLGVLRLLLF |
| | | KLLLFDLLLTCSCLCDPAGPLPSPATTTRLRALGSHRLHPATETGG |
| | | REATSSPRPQPRDRRWGDTPPGRKPGSPV |
| 439 | RAS/P53 | DKQLDADVSPKPTIFLPSIAETKLQKAGTYLCLLEKFFPDIIKIHWQ |
| | | EKKSNTILGSQEGNTMKTNDTYMKFSWLTVPEESLDKEHRCIVRH |
| | | ENNKNGIDQEIIFPPIKTDVTTVDPKYNYSKDANDVITMDPKDNW |
| | | SKDANDTLLLQLTNTSAYYMYLLLLLKSVVYFAIITCCLLGRTAFC |
| | | CNGEKS |
| 440 | RAS/P53 | DKQLDADVSPKPTIFLPSIAETKLQKAGTYLCLLEKFFPDVIKIHW |
| | | QEKKSNTILGSQEGNTMKTNDTYMKFSWLTVPEKSLDKEHRCIV |
| | | RHENNKNGVDQEIIFPPIKTDVITMDPKDNCSKDANDTLLLQLTNT |
| | | SAYYMYLLLLLKSVVYFAIITCCLLRRTAFCCNGEKS |
| 441 | RAS/P53 | SQPHTKPSVFVMKNGTNVACLVKEFYPKDIRINLVSSKKITEFDPA |
| | | IVISPSGKYNAVKLGKYEDSNSVTCSVQHDNKTVHSTDFEVKTDS |
| | | TDHVKPKETENTKQPSKSCHKPKAIVHTEKVNMMSLTVLGLRML |
| | | FAKTVAVNFLLTAKLFFL |
| 442 | RAS/P53 | SQPHTKPSVFVMKNGTNVACLVKEFYPKDIRINLVSSKKITEFDPA |
| | | IVISPSGKYNAVKLGKYEDSNSVTCSVQHDNKTVHSTDFEVKTDS |
| | | TDHVKPKETENTKQPSKSCHKPKAIVHTEKVNMMSLTVLGLRML |
| | | FAKTVAVNFLLTAKLFFL |
| 443 | RAS/P53 | IQNPDPAVYITDKTVLDMRSMDFKSNSAVAWSNKSDFACANAFN |
| | | NSIIPEDTFFPSPESSCDVKLVEKSFETDTNLNFQNLSVIGFRILLLK |
| | | VAGFNLLMTLRLWSS |
| 444 | RAS/P53 | EDLKNVFPPEVAVFEPSRAEISHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 445 | RAS/P53 | EDLKNVFPPEVAVFEPSRAEIAHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 446 | RAS/P53 | atccagaaccctgatcctgccgtgtaccagctgcgggattgcaagagcagcgacaagagcgtgtgtctg |
| | | ttcaccgacttcgacagccagaccaacgtgtcccagagcaaggactccgacgtgtacatcaccgataag |
| | | accgtgctggacatgcggagcatggacttcaagagcaactccgccgtggcctggtccaacaagtccgat |
| | | ttcgcctgcgccaacgccttcaacaacagcattatccccgaggacacattcttcccaagccccgagagca |
| | | gctgcgacgtgaagctggtggaaaagagcttcgagacagacaccaacctgaacttccagaacctgagc |
| | | gtgatcggcttcagaatcctgctgctgaaagtggccggctttaacctgctgatgaccctgcggctgtggtc |
| | | cagc |
| 447 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatctgcgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcgacgtgaagctggtggaaaagagcttcgagacagacaccaacctgaacttccagaacctga |
| | | gcgtgatcggcttcagaatcctgctgctgaaagtggccggctttaacctgctgatgaccctgcggctgtg |
| | | gtccagc |
| 448 | RAS/P53 | gaggacctgaagaacgtgttcccacctgaggtggccgtgttctgccctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 449 | RAS/P53 | gaggacctgaagaacgtgttcccacctgaggtggccgtgttcgagccttgcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 450 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctacctgcccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 451 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgtgcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggctttaccagcgag |
| | | agctaccagcagggcgtgctgagcgccaccatcctgtacgagatcctgctgggcaaggccaccctgta |
| | | cgccgtgctggtgtctgccctggtgctgatggctatggtcaagcggaaggacagccgggga |
| 452 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcgga |
| 453 | RAS/P53 | gaggacctgaagaacgtgttcccacctgaggtggccgtgttcgagcctagcaaggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtgtaccgacccccagccactgaaagaacagcccgcc |
| | | ctgaacgacagccggtactgcctgagcagcagactgagagtgtccgccaccttctggcagaacccccg |
| | | gaaccacttcagatgccaggtgcagttctacggcctgagcgagaacgacgagtggacccaggacaga |
| | | gccaagcccgtgacccagatcgtgtctgccgaagcctggggcagagccgattgc |
| 454 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtgtaccgacccccagccactgaaagaacagcccgcc |
| | | ctgaacgacagccggtactgcctgagcagcagactgagagtgtccgccaccttctggcagaacccccg |
| | | gaaccacttcagatgccaggtgcagttctacggcctgagcgagaacgacgagtggacccaggacaga |
| | | gccaagcccgtgacccagatcgtgtctgccgaagcctggggcagagccgattgc |
| 455 | RAS/P53 | cagagcttcggcctgctggaccccaagctgtgctacctgctggatggcatcctgttcatctacggcgtgat |
| | | cctgaccgccctgttcctg |
| 456 | RAS/P53 | cagagcttcggcctgctggaccccaagctgtgctacctgctggatggcatcctgttcatctacggcgtgat |
| | | cctgaccgccctgttcctgagagtgaagttcagcagaagcgccgacgcccctgcctaccagcagggac |
| | | agaaccaactgtacaacgagctgaacctgggcagacgggaagagtacgacgtgctggacaagcgga |
| | | gaggcagggaccctgagatgggcggaaagccccagcggagaaagaacccccaggaaggactgtat |
| | | aacgaactccagaaagacaagatggccgaagcctacagcgagatcggcatgaagggcgagcggaga |
| | | agaggcaagggccacgatggcctgtaccagggcctgagcaccgccaccaaggacacctatgacgcc |
| | | ctgcacatgcaggccctgcctccaaga |
| 457 | RAS/P53 | cagagcttcggcctgctggaccccaagctgtgctacctgctggatggcatcctgttcatctacggcgtgat |
| | | cctgaccgccctgttcctgagagtgaagttcagcagaagcgccgacgcccctgcctaccagcagggac |
| | | agaaccaactgtacaacgagctgaacctgggcagacgggaagagtacgacgtgctggacaagcgga |
| | | gaggcagggaccctgagatgggcggaaagccccagcggagaaagaacccccaggaaggactgtat |
| | | aacgaactccagaaagacaagatggccgaagcctacagcgagatcggcatgaagggcgagcggaga |
| | | agaggcaagggccacgatggcctgtaccagggcctgagcaccgccaccaaggacacctatgacgcc |
| | | ctgcacatgcaggccctgcctccaaga |
| 458 | RAS/P53 | aggagtaagaggagcaggctcctgcacagtgactacatgaacatgactccacgccgccccggaccca |
| | | cccgcaagcattaccagccctatgccccaccacgcgacttcgcagcctatcgctcc |
| 459 | RAS/P53 | aaacggggcagaaagaaactcctgtatatattcaaacaaccatttatgagaccagtacaaactactcaag |
| | | aggaagatggctgtagctgccgatttccagaagaagaagaaggaggatgtgaactg |
| 460 | RAS/P53 | aagaaccggaaggccaaggccaagcctgtgacaagaggtgctggtgctggcggcagacagagaggc |
| | | cagaacaaagaaagacctcctcctgtgcctaatcctgactacgagcccatcagaaaaggccagcggga |
| | | tctgtacagcggcctgaaccagcggcggatc |
| 461 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggatcccagagcttc |
| | | ggcctgctggaccccaagctgtgctacctgctggatggcatcctgttcatctacggcgtgatcctgaccg |
| | | ccctgttcctgagagtgaagttcagcagaagcgccgacgcccctgcctaccagcagggacagaaccaa |
| | | ctgtacaacgagctgaacctgggcagacgggaagagtacgacgtgctggacaagcggagaggcagg |
| | | gaccctgagatgggcggaaagccccagcggagaaagaacccccaggaaggactgtataacgaactc |
| | | cagaaagacaagatggccgaagcctacagcgagatcggcatgaagggcgagcggagaagaggcaa |
| | | gggccacgatggcctgtaccagggcctgagcaccgccaccaaggacacctatgacgccctgcacatg |
| | | caggccctgcctccaaga |
| 462 | RAS/P53 | gaggacctgaagaacgtgttcccacctgaggtggccgtgttcgagcctagcaaggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtgtaccgacccccagccactgaaagaacagcccgcc |
| | | ctgaacgacagccggtactgcctgagcagcagactgagagtgtccgccaccttctggcagaacccccg |
| | | gaaccacttcagatgccaggtgcagttctacggcctgagcgagaacgacgagtggacccaggacaga |
| | | gccaagcccgtgacccagatcgtgtctgccgaagcctggggcagagccgattgcggatcccagagctt |
| | | cggcctgctggaccccaagctgtgctacctgctggatggcatcctgttcatctacggcgtgatcctgacc |
| | | gccctgttcctgagagtgaagttcagcagaagcgccgacgcccctgcctaccagcagggacagaacca |
| | | actgtacaacgagctgaacctgggcagacgggaagagtacgacgtgctggacaagcggagaggcag |
| | | ggaccctgagatgggcggaaagccccagcggagaaagaacccccaggaaggactgtataacgaact |
| | | ccagaaagacaagatggccgaagcctacagcgagatcggcatgaagggcgagcggagaagaggca |
| | | agggccacgatggcctgtaccagggcctgagcaccgccaccaaggacacctatgacgccctgcacat |
| | | gcaggccctgcctccaaga |
| 463 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtgtaccgacccccagccactgaaagaacagcccgcc |
| | | ctgaacgacagccggtactgcctgagcagcagactgagagtgtccgccaccttctggcagaacccccg |
| | | gaaccacttcagatgccaggtgcagttctacggcctgagcgagaacgacgagtggacccaggacaga |
| | | gccaagcccgtgacccagatcgtgtctgccgaagcctggggcagagccgattgcggatcccagagctt |
| | | cggcctgctggaccccaagctgtgctacctgctggatggcatcctgttcatctacggcgtgatcctgacc |
| | | gccctgttcctgaaacggggcagaaagaaactcctgtatatattcaaacaaccatttatgagaccagtaca |
| | | aactactcaagaggaagatggctgtagctgccgatttccagaagaagaagaaggaggatgtgaactga |
| | | gagtgaagttcagcagaagcgccgacgcccctgcctaccagcagggacagaaccaactgtacaacga |
| | | gctgaacctgggcagacgggaagagtacgacgtgctggacaagcggagaggcagggaccctgagat |
| | | gggcggaaagccccagcggagaaagaacccccaggaaggactgtataacgaactccagaaagacaa |
| | | gatggccgaagcctacagcgagatcggcatgaagggcgagcggagaagaggcaagggccacgatg |
| | | gcctgtaccagggcctgagcaccgccaccaaggacacctatgacgccctgcacatgcaggccctgcct |
| | | ccaaga |
| 464 | RAS/P53 | gaggacctgaagaacgtgttcccacctgaggtggccgtgttctgccctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggatcccagagcttc |
| | | ggcctgctggaccccaagctgtgctacctgctggatggcatcctgttcatctacggcgtgatcctgaccg |
| | | ccctgttcctgaaacggggcagaaagaaactcctgtatatattcaaacaaccatttatgagaccagtacaa |
| | | actactcaagaggaagatggctgtagctgccgatttccagaagaagaagaaggaggatgtgaactgag |
| | | agtgaagttcagcagaagcgccgacgcccctgcctaccagcagggacagaaccaactgtacaacgag |
| | | ctgaacctgggcagacgggaagagtacgacgtgctggacaagcggagaggcagggaccctgagatg |
| | | ggcggaaagccccagcggagaaagaacccccaggaaggactgtataacgaactccagaaagacaag |
| | | atggccgaagcctacagcgagatcggcatgaagggcgagcggagaagaggcaagggccacgatgg |
| | | cctgtaccagggcctgagcaccgccaccaaggacacctatgacgccctgcacatgcaggccctgcctc |
| | | caaga |
| 465 | RAS/P53 | gaggacctgaagaacgtgttcccacctgaggtggccgtgttcgagccttgcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggatcccagagcttc |
| | | ggcctgctggaccccaagctgtgctacctgctggatggcatcctgttcatctacggcgtgatcctgaccg |
| | | ccctgttcctgaaacggggcagaaagaaactcctgtatatattcaaacaaccatttatgagaccagtacaa |
| | | actactcaagaggaagatggctgtagctgccgatttccagaagaagaagaaggaggatgtgaactgag |
| | | agtgaagttcagcagaagcgccgacgcccctgcctaccagcagggacagaaccaactgtacaacgag |
| | | ctgaacctgggcagacgggaagagtacgacgtgctggacaagcggagaggcagggaccctgagatg |
| | | ggcggaaagccccagcggagaaagaacccccaggaaggactgtataacgaactccagaaagacaag |
| | | atggccgaagcctacagcgagatcggcatgaagggcgagcggagaagaggcaagggccacgatgg |
| | | cctgtaccagggcctgagcaccgccaccaaggacacctatgacgccctgcacatgcaggccctgcctc |
| | | caaga |
| 466 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctacctgcccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggatcccagagcttc |
| | | ggcctgctggaccccaagctgtgctacctgctggatggcatcctgttcatctacggcgtgatcctgaccg |
| | | ccctgttcctgaaacggggcagaaagaaactcctgtatatattcaaacaaccatttatgagaccagtacaa |
| | | actactcaagaggaagatggctgtagctgccgatttccagaagaagaagaaggaggatgtgaactgag |
| | | agtgaagttcagcagaagcgccgacgcccctgcctaccagcagggacagaaccaactgtacaacgag |
| | | ctgaacctgggcagacgggaagagtacgacgtgctggacaagcggagaggcagggaccctgagatg |
| | | ggcggaaagccccagcggagaaagaacccccaggaaggactgtataacgaactccagaaagacaag |
| | | atggccgaagcctacagcgagatcggcatgaagggcgagcggagaagaggcaagggccacgatgg |
| | | cctgtaccagggcctgagcaccgccaccaaggacacctatgacgccctgcacatgcaggccctgcctc |
| | | caaga |
| 467 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgtgcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggatcccagagcttc |
| | | ggcctgctggaccccaagctgtgctacctgctggatggcatcctgttcatctacggcgtgatcctgaccg |
| | | ccctgttcctgaaacggggcagaaagaaactcctgtatatattcaaacaaccatttatgagaccagtacaa |
| | | actactcaagaggaagatggctgtagctgccgatttccagaagaagaagaaggaggatgtgaactgag |
| | | agtgaagttcagcagaagcgccgacgcccctgcctaccagcagggacagaaccaactgtacaacgag |
| | | ctgaacctgggcagacgggaagagtacgacgtgctggacaagcggagaggcagggaccctgagatg |
| | | ggcggaaagccccagcggagaaagaacccccaggaaggactgtataacgaactccagaaagacaag |
| | | atggccgaagcctacagcgagatcggcatgaagggcgagcggagaagaggcaagggccacgatgg |
| | | cctgtaccagggcctgagcaccgccaccaaggacacctatgacgccctgcacatgcaggccctgcctc |
| | | caaga |
| 468 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggatcccagagcttc |
| | | ggcctgctggaccccaagctgtgctacctgctggatggcatcctgttcatctacggcgtgatcctgaccg |
| | | ccctgttcctgaggagtaagaggagcaggctcctgcacagtgactacatgaacatgactccacgccgcc |
| | | ccggacccacccgcaagcattaccagccctatgccccaccacgcgacttcgcagcctatcgctccaga |
| | | gtgaagttcagcagaagcgccgacgcccctgcctaccagcagggacagaaccaactgtacaacgagc |
| | | tgaacctgggcagacgggaagagtacgacgtgctggacaagcggagaggcagggaccctgagatgg |
| | | gcggaaagccccagcggagaaagaacccccaggaaggactgtataacgaactccagaaagacaaga |
| | | tggccgaagcctacagcgagatcggcatgaagggcgagcggagaagaggcaagggccacgatggc |
| | | ctgtaccagggcctgagcaccgccaccaaggacacctatgacgccctgcacatgcaggccctgcctcc |
| | | aaga |
| 469 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggatcccagagcttc |
| | | ggcctgctggaccccaagctgtgctacctgctggatggcatcctgttcatctacggcgtgatcctgaccg |
| | | ccctgttcctgaaacggggcagaaagaaactcctgtatatattcaaacaaccatttatgagaccagtacaa |
| | | actactcaagaggaagatggctgtagctgccgatttccagaagaagaagaaggaggatgtgaactgag |
| | | agtgaagttcagcagaagcgccgacgcccctgcctaccagcagggacagaaccaactgtacaacgag |
| | | ctgaacctgggcagacgggaagagtacgacgtgctggacaagcggagaggcagggaccctgagatg |
| | | ggcggaaagccccagcggagaaagaacccccaggaaggactgtataacgaactccagaaagacaag |
| | | atggccgaagcctacagcgagatcggcatgaagggcgagcggagaagaggcaagggccacgatgg |
| | | cctgtaccagggcctgagcaccgccaccaaggacacctatgacgccctgcacatgcaggccctgcctc |
| | | caaga |
| 470 | RAS/P53 | gaggacctgaagaacgtgttcccacctgaggtggccgtgttcgagcctagcgaggccgagatcagcca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggatcccagagcttc |
| | | ggcctgctggaccccaagctgtgctacctgctggatggcatcctgttcatctacggcgtgatcctgaccg |
| | | ccctgttcctgaaacggggcagaaagaaactcctgtatatattcaaacaaccatttatgagaccagtacaa |
| | | actactcaagaggaagatggctgtagctgccgatttccagaagaagaagaaggaggatgtgaactgaa |
| | | gaaccggaaggccaaggccaagcctgtgacaagaggtgctggtgctggcggcagacagagaggcca |
| | | gaacaaagaaagacctcctcctgtgcctaatcctgactacgagcccatcagaaaaggccagcgggatct |
| | | gtacagcggcctgaaccagcggcggatc |
| 471 | RAS/P53 | atccagaaccctgatcctgccgtgtaccagctggggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgc |
| 472 | RAS/P53 | atccagaaccctgatcctgccgtgtaccagctggggatagcaagagcagcgacaagagcgtgtgtct |
| | | gttcaccgacttcgacagccagaccaacgtgtcccagagcaaggactccgacgtgtacatcaccgataa |
| | | gtgcgtgctggacatgcggagcatggacttcaagagcaactccgccgtggcctggtccaacaagtccg |
| | | atttcgcctgcgccaacgccttcaacaacagcattatccccgaggacacattcttcccaagcaggacgtg |
| | | ccctgc |
| 473 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gtgcgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgc |
| 474 | RAS/P53 | cagagttttgggttgttggaccctaaattgtgctatctgttggacggaatcctgttcatttacggagtgatc |
| | | ctgaccgcattgttcctg |
| 475 | RAS/P53 | cgggttaagttttccagatccgcagacgcacccgcttaccaacaagggcagaaccaactctacaatgaa |
| | | cttaatttggggcggagggaagagtacgacgttctggacaaaaggaggggacgagatcccgagatgg |
| | | gcggaaagcctcagcgaaggaagaatccgcaagaggggctttataacgaactccagaaagataaaatg |
| | | gcagaagcctatagtgagattgggatgaagggagaaaggaggaggggtaaaggtcacgatgggttgt |
| | | accaaggattgagcacagccactaaagatacctatgatgcgctccacatgcaggccctgccgccaaga |
| 476 | RAS/P53 | cagagttttgggttgttggaccctaaattgtgctatctgttggacggaatcctgttcatttacggagtgatcc |
| | | tgaccgcattgttcctgcgggttaagttttccagatccgcagacgcacccgcttaccaacaagggcagaac |
| | | caactctacaatgaacttaatttggggcggagggaagagtacgacgttctggacaaaaggaggggacg |
| | | agatcccgagatgggcggaaagcctcagcgaaggaagaatccgcaagaggggctttataacgaactc |
| | | cagaaagataaaatggcagaagcctatagtgagattgggatgaagggagaaaggaggaggggtaaag |
| | | gtcacgatgggttgtaccaaggattgagcacagccactaaagatacctatgatgcgctccacatgcaggc |
| | | cctgccgccaaga |
| 477 | RAS/P53 | aggagcaagcggtcacggctgctccacagcgattacatgaatatgacaccacgaagaccgggtcctac |
| | | tcgcaaacactaccaaccctacgcacctccccgcgactttgcagcgtaccgatct |
| 478 | RAS/P53 | aagagaggccggaagaagctgctgtacatcttcaagcagcctttcatgcggcccgtgcagaccaccca |
| | | ggaagaggacggctgctcctgccggttccccgaggaagaagaaggcggctgcgagctg |
| 479 | RAS/P53 | aagaatagaaaggccaaagctaaacccgtgaccagaggcgctggcgcaggcggaaggcaaagggg |
| | | acaaaacaaagaacggccaccaccagtgcaaaatccagattatgagcctattcggaaaggacagaggg |
| | | acctgtactctggcctgaatcagagaagaatc |
| 480 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcggtacccagagttttgggttgttggaccctaaattgtgctatctgttggacggaatcctgttcatt |
| | | tacggagtgatcctgaccgcattgttcctgcgggttaagttttccagatccgcagacgcacccgcttaccaa |
| | | caagggcagaaccaactctacaatgaacttaatttggggcggagggaagagtacgacgttctggacaaa |
| | | aggaggggacgagatcccgagatgggcggaaagcctcagcgaaggaagaatccgcaagaggggct |
| | | ttataacgaactccagaaagataaaatggcagaagcctatagtgagattgggatgaagggagaaagga |
| | | ggaggggtaaaggtcacgatgggttgtaccaaggattgagcacagccactaaagatacctatgatgcgc |
| | | tccacatgcaggccctgccgccaaga |
| 481 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gtgcgtgctggacatgcggagcatggacttcaagagcaactccgccgtggcctggtccaacaagtccg |
| | | atttcgcctgcgccaacgccttcaacaacagcattatccccgaggacacattcttcccaagcagcgacgt |
| | | gccctgcggtacccagagttttgggttgttggaccctaaattgtgctatctgttggacggaatcctgttcatt |
| | | tacggagtgatcctgaccgcattgttcctgcgggttaagttttccagatccgcagacgcacccgcttaccaa |
| | | caagggcagaaccaactctacaatgaacttaatttggggcggagggaagagtacgacgttctggacaaa |
| | | aggaggggacgagatcccgagatgggggaaagcctcagcgaaggaagaatccgcaagaggggct |
| | | ttataacgaactccagaaagataaaatggcagaagcctatagtgagattgggatgaagggagaaagga |
| | | ggaggggtaaaggtcacgatgggttgtaccaaggattgagcacagccactaaagatacctatgatgcgc |
| | | tccacatgcaggccctgccgccaaga |
| 482 | RAS/P53 | atccagaaccctgatcctgccgtgtaccagctggggatagcaagagcagcgacaagagcgtgtgtct |
| | | gttcaccgacttcgacagccagaccaacgtgtcccagagcaaggactccgacgtgtacatcaccgataa |
| | | gtgcgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcggtacccagagttttgggttgttggaccctaaattgtgctatctgttggacggaatcctgttcatt |
| | | tacggagtgatcctgaccgcattgttcctgcgggttaagttttccagatccgcagacgcacccgcttaccaa |
| | | caagggcagaaccaactctacaatgaacttaatttggggcggagggaagagtacgacgttctggacaaa |
| | | aggaggggacgagatcccgagatgggcggaaagcctcagcgaaggaagaatccgcaagaggggct |
| | | ttataacgaactccagaaagataaaatggcagaagcctatagtgagattgggatgaagggagaaagga |
| | | ggaggggtaaaggtcacgatgggttgtaccaaggattgagcacagccactaaagatacctatgatgcgc |
| | | tccacatgcaggccctgccgccaaga |
| 483 | RAS/P53 | atccagaaccctgatcctgccgtgtgccagctggggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcggtacccagagttttgggttgttggaccctaaattgtgctatctgttggacggaatcctgttcatt |
| | | tacggagtgatcctgaccgcattgttcctgcgggttaagttttccagatccgcagacgcacccgcttaccaa |
| | | caagggcagaaccaactctacaatgaacttaatttggggggagggaagagtacgacgttctggacaaa |
| | | aggaggggacgagatcccgagatgggcggaaagcctcagcgaaggaagaatccgcaagaggggct |
| | | ttataacgaactccagaaagataaaatggcagaagcctatagtgagattgggatgaagggagaaagga |
| | | ggaggggtaaaggtcacgatgggttgtaccaaggattgagcacagccactaaagatacctatgatgcgc |
| | | tccacatgcaggccctgccgccaaga |
| 484 | RAS/P53 | atccagaaccctgatcctgccgtgtaccagctggggattgcaagagcagcgacaagagcgtgtgtctg |
| | | ttcaccgacttcgacagccagaccaacgtgtcccagagcaaggactccgacgtgtacatcaccgataag |
| | | accgtgctggacatgcggagcatggacttcaagagcaactccgccgtggcctggtccaacaagtccgat |
| | | ttcgcctgcgccaacgccttcaacaacagcattatccccgaggacacattcttcccaagccccgagagca |
| | | gctgcggtacccagagttttgggttgttggaccctaaattgtgctatctgttggacggaatcctgttcattta |
| | | cggagtgatcctgaccgcattgttcctgcgggttaagttttccagatccgcagacgcacccgcttaccaaca |
| | | agggcagaaccaactctacaatgaacttaatttggggcggagggaagagtacgacgttctggacaaaa |
| | | ggaggggacgagatcccgagatgggcggaaagcctcagcgaaggaagaatccgcaagaggggcttt |
| | | ataacgaactccagaaagataaaatggcagaagcctatagtgagattgggatgaagggagaaaggagg |
| | | aggggtaaaggtcacgatgggttgtaccaaggattgagcacagccactaaagatacctatgatgcgctc |
| | | cacatgcaggccctgccgccaaga |
| 485 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatctgcgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcggtacccagagttttgggttgttggaccctaaattgtgctatctgttggacggaatcctgttcatt |
| | | tacggagtgatcctgaccgcattgttcctgcgggttaagttttccagatccgcagacgcacccgcttaccaa |
| | | caagggcagaaccaactctacaatgaacttaatttggggcggagggaagagtacgacgttctggacaaa |
| | | aggaggggacgagatcccgagatgggcggaaagcctcagcgaaggaagaatccgcaagaggggct |
| | | ttataacgaactccagaaagataaaatggcagaagcctatagtgagattgggatgaagggagaaagga |
| | | ggaggggtaaaggtcacgatgggttgtaccaaggattgagcacagccactaaagatacctatgatgcgc |
| | | tccacatgcaggccctgccgccaaga |
| 486 | RAS/P53 | atccagaaccctgatcctgccgtgtaccagctggggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcggtacccagagttttgggttgttggaccctaaattgtgctatctgttggacggaatcctgttcatt |
| | | tacggagtgatcctgaccgcattgttcctgaggagcaagcggtcacggctgctccacagcgattacatga |
| | | atatgacaccacgaagaccgggtcctactcgcaaacactaccaaccctacgcacctccccgcgactttg |
| | | cagcgtaccgatctcgggttaagttttccagatccgcagacgcacccgcttaccaacaagggcagaacc |
| | | aactctacaatgaacttaatttggggcggagggaagagtacgacgttctggacaaaaggaggggacga |
| | | gatcccgagatgggcggaaagcctcagcgaaggaagaatccgcaagaggggctttataacgaactcc |
| | | agaaagataaaatggcagaagcctatagtgagattgggatgaagggagaaaggaggaggggtaaagg |
| | | tcacgatgggttgtaccaaggattgagcacagccactaaagatacctatgatgcgctccacatgcaggcc |
| | | ctgccgccaaga |
| 487 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcggtacccagagttttgggttgttggaccctaaattgtgctatctgttggacggaatcctgttcatt |
| | | tacggagtgatcctgaccgcattgttcctgaagagaggccggaagaagctgctgtacatcttcaagcagc |
| | | ctttcatgcggcccgtgcagaccacccaggaagaggacggctgctcctgccggttccccgaggaagaa |
| | | gaaggcggctgcgagctgcgggttaagttttccagatccgcagacgcacccgcttaccaacaagggca |
| | | gaaccaactctacaatgaacttaatttggggcggagggaagagtacgacgttctggacaaaaggaggg |
| | | gacgagatcccgagatgggcggaaagcctcagcgaaggaagaatccgcaagaggggctttataacga |
| | | actccagaaagataaaatggcagaagcctatagtgagattgggatgaagggagaaaggaggaggggt |
| | | aaaggtcacgatgggttgtaccaaggattgagcacagccactaaagatacctatgatgcgctccacatgc |
| | | aggccctgccgccaaga |
| 488 | RAS/P53 | atccagaaccctgatcctgccgtgtaccagctgcgggatagcaagagcagcgacaagagcgtgtgtct |
| | | gttcaccgacttcgacagccagaccaacgtgtcccagagcaaggactccgacgtgtacatcaccgataa |
| | | gaccgtgctggacatgcggagcatggacttcaagagcaactccgccgtggcctggtccaacaagtccg |
| | | atttcgcctgcgccaacgccttcaacaacagcattatccccgaggacacattcttcccaagccccgagag |
| | | cagctgcggtacccagagttttgggttgttggaccctaaattgtgctatctgttggacggaatcctgttcatt |
| | | tacggagtgatcctgaccgcattgttcctgaagagaggccggaagaagctgctgtacatcttcaagcagc |
| | | ctttcatgcggcccgtgcagaccacccaggaagaggacggctgctcctgccggttccccgaggaagaa |
| | | gaaggcggctgcgagctgaagaatagaaaggccaaagctaaacccgtgaccagaggcgctggcgca |
| | | ggcggaaggcaaaggggacaaaacaaagaacggccaccaccagtgcaaaatccagattatgagccta |
| | | ttcggaaaggacagagggacctgtactctggcctgaatcagagaagaatc |
| 489 | RAS/P53 | IQNPDPAVCQLRDSKSSDKSVCLFTDFDSQTNVSQSKDSDVYITDK |
| | | TVLDMRSMDFKSNSAVAWSNKSDFACANAFNNSIIPEDTFFPSPES |
| | | SCDVKLVEKSFETDTNLNFQNLSVIGFRILLLKVAGFNLLMTLRL |
| | | WSS |
| 490 | RAS/P53 | IQNPDPAVYQLRDCKSSDKSVCLFTDFDSQTNVSQSKDSDVYITD |
| | | KTVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 491 | RAS/P53 | IQNPDPAVYQLRDSKSSDKSVCLFTDFDSQTNVSQSKDSDVYICD |
| | | KTVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 492 | RAS/P53 | EDLKNVFPPEVAVFCPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 493 | RAS/P53 | EDLKNVFPPEVAVFEPCEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 494 | RAS/P53 | EDLKNVFPPEVAVFEPSEAEISHTQKATLVCLATGFYPDHVELSW |
| | | WVNGKEVHSGVSTCPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 495 | RAS/P53 | EDLKNVFPPEVAVFEPSEAEISHTQKATLVCLATGFYPDHVELSW |
| | | WVNGKEVHSGVSTDPQPLKEQPALNDSRYCLCSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 496 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCG |
| 497 | RAS/P53 | EDLKNVFPPEVAVFEPSKAEIAHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADC |
| 498 | RAS/P53 | EDLKNVFPPEVAVFEPSEAEISHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADC |
| 499 | RAS/P53 | QSFGLLDPKLCYLLDGILFIYGVILTALFL |
| 500 | RAS/P53 | QSFGLLDPKLCYLLDGILFIYGVILTALFLRVKFSRSADAPAYQQG |
| | | QNQLYNELNLGRREEYDVLDKRRGRDPEMGGKPQRRKNPQEGL |
| | | YNELQKDKMAEAYSEIGMKGERRRGKGHDGLYQGLSTATKDTY |
| | | DALHMQALPPR |
| 501 | RAS/P53 | QSFGLLDPKLCYLLDGILFIYGVILTALFLRVKFSRSADAPAYQQG |
| | | QNQLYNELNLGRREEYDVLDKRRGRDPEMGGKPQRRKNPQEGL |
| | | YNELQKDKMAEAYSEIGMKGERRRGKGHDGLYQGLSTATKDTY |
| | | DALHMQALPPR |
| 502 | RAS/P53 | RSKRSRLLHSDYMNMTPRRPGPTRKHYQPYAPPRDFAAYRS |
| 503 | RAS/P53 | KRGRKKLLYIFKQPFMRPVQTTQEEDGCSCRFPEEEEGGCEL |
| 504 | RAS/P53 | KNRKAKAKPVTRGAGAGGRQRGQNKERPPPVPNPDYEPIRKGQR |
| | | DLYSGLNQRRI |
| 505 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLRVKFSRSADAPAYQQG |
| | | QNQLYNELNLGRREEYDVLDKRRGRDPEMGGKPQRRKNPQEGL |
| | | YNELQKDKMAEAYSEIGMKGERRRGKGHDGLYQGLSTATKDTY |
| | | DALHMQALPPR |
| 506 | RAS/P53 | EDLKNVFPPEVAVFEPSKAEIAHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLRVKFSRSADAPAYQQG |
| | | QNQLYNELNLGRREEYDVLDKRRGRDPEMGGKPQRRKNPQEGL |
| | | YNELQKDKMAEAYSEIGMKGERRRGKGHDGLYQGLSTATKDTY |
| | | DALHMQALPPR |
| 507 | RAS/P53 | EDLKNVFPPEVAVFEPATGFYPDHVELSWWVNGKEVHSGVCTDP |
| | | QPLKEQPALNDSRYCLSSRLRVSATFWQNPRNHFRCQVQFYGLSE |
| | | NDEWTQDRAKPVTQIVSAEAWGRADCGSQSFGLLDPKLCYLLDG |
| | | ILFIYGVILTALFLKRGRKKLLYIFKQPFMRPVQTTQEEDGCSCRFP |
| | | EEEEGGCELRVKFSRSADAPAYQQGQNQLYNELNLGRREEYDVL |
| | | DKRRGRDPEMGGKPQRRKNPQEGLYNELQKDKMAEAYSEIGMK |
| | | GERRRGKGHDGLYQGLSTATKDTYDALHMQALPPR |
| 508 | RAS/P53 | EDLKNVFPPEVAVFCPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLKRGRKKLLYIFKQPFMR |
| | | PVQTTQEEDGCSCRFPEEEEGGCELRVKFSRSADAPAYQQGQNQL |
| | | YNELNLGRREEYDVLDKRRGRDPEMGGKPQRRKNPQEGLYNEL |
| | | QKDKMAEAYSEIGMKGERRRGKGHDGLYQGLSTATKDTYDALH |
| | | MQALPPR |
| 509 | RAS/P53 | EDLKNVFPPEVAVFEPCEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLKRGRKKLLYIFKQPFMR |
| | | PVQTTQEEDGCSCRFPEEEEGGCELRVKFSRSADAPAYQQGQNQL |
| | | YNELNLGRREEYDVLDKRRGRDPEMGGKPQRRKNPQEGLYNEL |
| | | QKDKMAEAYSEIGMKGERRRGKGHDGLYQGLSTATKDTYDALH |
| | | MQALPPR |
| 510 | RAS/P53 | EDLKNVFPPEVAVFEPSEAEISHTQKATLVCLATGFYPDHVELSW |
| | | WVNGKEVHSGVSTCPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLKRGRKKLLYIFKQPFMR |
| | | PVQTTQEEDGCSCRFPEEEEGGCELRVKFSRSADAPAYQQGQNQL |
| | | YNELNLGRREEYDVLDKRRGRDPEMGGKPQRRKNPQEGLYNEL |
| | | QKDKMAEAYSEIGMKGERRRGKGHDGLYQGLSTATKDTYDALH |
| | | MQALPPR |
| 511 | RAS/P53 | EDLKNVFPPEVAVFEPSEAEISHTQKATLVCLATGFYPDHVELSW |
| | | WVNGKEVHSGVSTDPQPLKEQPALNDSRYCLCSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLKRGRKKLLYIFKQPFMR |
| | | PVQTTQEEDGCSCRFPEEEEGGCELRVKFSRSADAPAYQQGQNQL |
| | | YNELNLGRREEYDVLDKRRGRDPEMGGKPQRRKNPQEGLYNEL |
| | | QKDKMAEAYSEIGMKGERRRGKGHDGLYQGLSTATKDTYDALH |
| | | MQALPPR |
| 512 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLRSKRSRLLHSDYMNMT |
| | | PRRPGPTRKHYQPYAPPRDFAAYRSRVKFSRSADAPAYQQGQNQ |
| | | LYNELNLGRREEYDVLDKRRGRDPEMGGKPQRRKNPQEGLYNEL |
| | | QKDKMAEAYSEIGMKGERRRGKGHDGLYQGLSTATKDTYDALH |
| | | MQALPPR |
| 513 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLKRGRKKLLYIFKQPFMR |
| | | PVQTTQEEDGCSCRFPEEEEGGCELRVKFSRSADAPAYQQGQNQL |
| | | YNELNLGRREEYDVLDKRRGRDPEMGGKPQRRKNPQEGLYNEL |
| | | QKDKMAEAYSEIGMKGERRRGKGHDGLYQGLSTATKDTYDALH |
| | | MQALPPR |
| 514 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLKRGRKKLLYIFKQPFMR |
| | | PVQTTQEEDGCSCRFPEEEEGGCELKNRKAKAKPVTRGAGAGGR |
| | | QRGQNKERPPPVPNPDYEPIRKGQRDLYSGLNQRRI |
| 515 | RAS/P53 | IQNPDPAVYQLRDSKSSDKSVCLFTDFDSQTNVSQSKDSDVYITD |
| | | KCVLDMRSMDFKSNSAVAWSNKSDFACANAFNNSIIPEDTFFPSP |
| | | ESSCGTQSFGLLDPKLCYLLDGILFIYGVILTALFLRVKFSRSADAP |
| | | AYQQGQNQLYNELNLGRREEYDVLDKRRGRDPEMGGKPQRRKN |
| | | PQEGLYNELQKDKMAEAYSEIGMKGERRRGKGHDGLYQGLSTA |
| | | TKDTYDALHMQALPPR |
| 516 | RAS/P53 | IQNPDPAVCQLRDSKSSDKSVCLFTDFDSQTNVSQSKDSDVYITDK |
| | | TVLDMRSMDFKSNSAVAWSNKSDFACANAFNNSIIPEDTFFPSPES |
| | | SCGTQSFGLLDPKLCYLLDGILFIYGVILTALFLRVKFSRSADAPAY |
| | | QQGQNQLYNELNLGRREEYDVLDKRRGRDPEMGGKPQRRKNPQ |
| | | EGLYNELQKDKMAEAYSEIGMKGERRRGKGHDGLYQGLSTATK |
| | | DTYDALHMQALPPR |
| 517 | RAS/P53 | IQNPDPAVYQLRDCKSSDKSVCLFTDFDSQTNVSQSKDSDVYITD |
| | | KTVLDMRSMDFKSNSAVAWSNKSDFACANAFNNSIIPEDTFFPSP |
| | | ESSCGTQSFGLLDPKLCYLLDGILFIYGVILTALFLRVKFSRSADAP |
| | | AYQQGQNQLYNELNLGRREEYDVLDKRRGRDPEMGGKPQRRKN |
| | | PQEGLYNELQKDKMAEAYSEIGMKGERRRGKGHDGLYQGLSTA |
| | | TKDTYDALHMQALPPR |
| 518 | RAS/P53 | IQNPDPAVYQLRDSKSSDKSVCLFTDFDSQTNVSQSKDSDVYICD |
| | | KTVLDMRSMDFKSNSAVAWSNKSDFACANAFNNSIIPEDTFFPSP |
| | | ESSCGTQSFGLLDPKLCYLLDGILFIYGVILTALFLRVKFSRSADAP |
| | | AYQQGQNQLYNELNLGRREEYDVLDKRRGRDPEMGGKPQRRKN |
| | | PQEGLYNELQKDKMAEAYSEIGMKGERRRGKGHDGLYQGLSTA |
| | | TKDTYDALHMQALPPR |
| 519 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCGTQSFGLLDPKLCYLLDGILFIYGVILTALFLRSKRSRLLHSD |
| | | YMNMTPRRPGPTRKHYQPYAPPRDFAAYRSRVKFSRSADAPAY |
| | | QQGQNQLYNELNLGRREEYDVLDKRRGRDPEMGGKPQRRKNPQ |
| | | EGLYNELQKDKMAEAYSEIGMKGERRRGKGHDGLYQGLSTATK |
| | | DTYDALHMQALPPR |
| 520 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCGTQSFGLLDPKLCYLLDGILFIYGVILTALFLKRGRKKLLYIF |
| | | KQPFMRPVQTTQEEDGCSCRFPEEEEGGCELRVKFSRSADAPA |
| | | YQQGQNQLYNELNLGRREEYDVLDKRRGRDPEMGGKPQRRKNP |
| | | QEGLYNELQKDKMAEAYSEIGMKGERRRGKGHDGLYQGLSTAT |
| | | KDTYDALHMQALPPR |
| 521 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCGTQSFGLLDPKLCYLLDGILFIYGVILTALFLKRGRKKLLYIF |
| | | KQPFMRPVQTTQEEDGCSCRFPEEEEGGCELKNRKAKAKPVTRG |
| | | AGAGGRQRGQNKERPPPVQNPDYEPIRKGQRDLYSGLNQRRI |
| 522 | RAS/P53 | IQNPDPAVYQLRDCKSSDKSVCLFTDFDSQTNVSQSKDSDVYITD |
| | | KTVLDMRSMDFKSNSAVAWSNKSDFACANAFNNSIIPEDTFFPSS |
| | | DVPCDVKLVEKSFETDTNLNFQNLSVIGFRILLLKVAGFNLLMTLR |
| | | LWSS |
| 523 | RAS/P53 | IQNPDPAVYQLRDCKSSDKSVCLFTDFDSQTNVSQSKDSDVYITD |
| | | KCVLDMRSMDFKSNSAVAWSNKSDFACANAFNNSIIPEDTFFPSS |
| | | DVPCDVKLVEKSFETDTNLNFQNLSVIGFRILLLKVAGFNLLMTLR |
| | | LWSS |
| 524 | RAS/P53 | EDLKNVFPPEVAVFCPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDSR |
| | | G |
| 525 | RAS/P53 | EDLKNVFPPEVAVFCPSKAEIAHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDSR |
| | | G |
| 526 | RAS/P53 | atccagaaccctgatcctgccgtgtaccagctgcgggattgcaagagcagcgacaagagcgtgtgtctg |
| | | ttcaccgacttcgacagccagaccaacgtgtcccagagcaaggactccgacgtgtacatcaccgataag |
| | | accgtgctggacatgcggagcatggacttcaagagcaactccgccgtggcctggtccaacaagtccgat |
| | | ttcgcctgcgccaacgccttcaacaacagcattatccccgaggacacattcttcccaagcagcgacgtgc |
| | | cctgcgacgtgaagctggtggaaaagagcttcgagacagacaccaacctgaacttccagaacctgagc |
| | | gtgatcggcttcagaatcctgctgctgaaagtggccggctttaacctgctgatgaccctgcggctgtggtc |
| | | cagc |
| 527 | RAS/P53 | atccagaaccctgatcctgccgtgtaccagctggggattgcaagagcagcgacaagagcgtgtgtctg |
| | | ttcaccgacttcgacagccagaccaacgtgtcccagagcaaggactccgacgtgtacatcaccgataag |
| | | tgcgtgctggacatgcggagcatggacttcaagagcaactccgccgtggcctggtccaacaagtccgat |
| | | ttcgcctgcgccaacgccttcaacaacagcattatccccgaggacacattcttcccaagcagcgacgtgc |
| | | cctgcgacgtgaagctggtggaaaagagcttcgagacagacaccaacctgaacttccagaacctgagc |
| | | gtgatcggcttcagaatcctgctgctgaaagtggccggctttaacctgctgatgaccctgcggctgtggtc |
| | | cagc |
| 528 | RAS/P53 | gaggacctgaagaacgtgttcccacctgaggtggccgtgttctgccctagcaaggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtctaccgacccccagccactgaaagaacagcccgccc |
| | | tgaacgacagccggtactgcctgagcagcagactgagagtgtccgccaccttctggcagaacccccgg |
| | | aaccacttcagatgccaggtgcagttctacggcctgagcgagaacgacgagtggacccaggacagag |
| | | ccaagcccgtgacccagatcgtgtctgccgaagcctggggcagagccgattgcggcatcaccagcgc |
| | | cagctaccaccagggcgtgctgagcgccaccatcctgtacgagatcctgctgggcaaggccaccctgt |
| | | acgccgtgctggtgtctgccctggtgctgatggctatggtcaagcggaaggacagccgggga |
| 529 | RAS/P53 | gaggacctgaagaacgtgttcccacctgaggtggccgtgttctgccctagcaaggccgagatcgccca |
| | | cacccagaaagccaccctcgtgtgcctggctaccggcttctaccccgaccatgtggaactgtcttggtgg |
| | | gtcaacggcaaagaggtgcacagcggcgtgtgtaccgacccccagccactgaaagaacagcccgcc |
| | | ctgaacgacagccggtactgcctgagcagcagactgagagtgtccgccaccttctggcagaacccccg |
| | | gaaccacttcagatgccaggtgcagttctacggcctgagcgagaacgacgagtggacccaggacaga |
| | | gccaagcccgtgacccagatcgtgtctgccgaagcctggggcagagccgattgcggcatcaccagcg |
| | | ccagctaccaccagggcgtgctgagcgccaccatcctgtacgagatcctgctgggcaaggccaccctg |
| | | tacgccgtgctggtgtctgccctggtgctgatggctatggtcaagcggaaggacagccgggga |
| 530 | RAS/P53 | cagtctgtgctgacacagcctcctagtgtgtctggcgctcctggacagagagtgaccatcagctgtacag |
| | | gcagcagcagcaatatcggagccggctatgacgtgcactggtatcagcagctgcctggcacagcccct |
| | | aaactgctgatctacggcaacagcaacagacccagcggcgtgcccgatagattttccggctctaagagc |
| | | ggcacaagcgccagcctggctattactggactccaggccgaggacgaggccgactactactgtcagag |
| | | ctacgacagcagcctgagcggctctgtgtttggcacaggcacaaaggtgctggtgctt |
| 531 | RAS/P53 | aacttcatgctgacccagcctcacagcgtgtccgagtctccaggcaagaccgtgaccatcagctgtaca |
| | | ggcagcagcggctctatcgccagcaactacgtgcagtggtatcagcagaggccaggcagcgccccta |
| | | ccatcctgatctacgaggataacaagcggcctagcggcgtgcccgatagattttctggcagcatcgaca |
| | | gcagcagcaacagcgccagcctgacaatcagcggcctgaaaacaggcgacgaggccgactactact |
| | | gccagagctacgacgacatcaaccactgggttttcggcggaggcaccaagctgacagttcttgga |
| 532 | RAS/P53 | QSVLTQPPSVSGAPGQRVTISCTGSSSNIGAGYDVHWYQQLPGTA |
| | | PKLLIYGNSNRPSGVPDRFSGSKSGTSASLAITGLQAEDEADYYCQ |
| | | SYDSSLSGSVFGTGTKVLVL |
| 533 | RAS/P53 | NFMLTQPHSVSESPGKTVTISCTGSSGSIASNYVQWYQQRPGSAPT |
| | | ILIYEDNKRPSGVPDRFSGSIDSSSNSASLTISGLKTGDEADYYCQS |
| | | YDDINHWVFGGGTKLTVLGQ |
| 534 | RAS/P53 | SSNIGAGYD |
| 535 | RAS/P53 | SGSIASNY |
| 536 | RAS/P53 | GNSN |
| 537 | RAS/P53 | EDNK |
| 538 | RAS/P53 | QSYDSSLSGSV |
| 539 | RAS/P5 | QSYDDINHWV |
| 540 | RAS/P53 | caagttcagctggttcagagcggagccgaagtgaagaaacctggcagcagcgtgaaggtgtcctgcaa |
| | | agcaagcggcggcaccttcagcagctacaccatcaacgtttggagacaggccccaggccagggcctt |
| | | gaatggatgggaggcttcatccctatcagcggcaccgtgaactacgcccagaaattccagggcagagt |
| | | gacaatcaccgccgacgagagcacaagcaccgcctacatggaactgtccagcctgagaagcgaggac |
| | | accgccgtgtactactgtgccagacctctggattggaccgaggacatctggggccagggaacactggtc |
| | | acagtgtctagt |
| 541 | RAS/P53 | gaagttcagctggttcagagcggagccgaagtgaagaaacctggggcctctgtgaaggtgtcctgcaa |
| | | ggcttccggctacacctttaccgcctactacctgcactggctgagacaggctccaggccaaggactgga |
| | | atggatgggctggatgaacaccaacaacggggccaccagatacgcccagaaattccaggacagagtg |
| | | accatgaccagagacaccagcatcaacaccgcctacatggaaatgagcggcctgtcctccgacgacac |
| | | cgccatgtactattgtgccagaggcgacatcagccaggacttcgctgatgtgtggggccagggaacact |
| | | ggtcacagtgtcatct |
| 542 | RAS/P53 | QVQLVQSGAEVKKPGSSVKVSCKASGGTFSSYTINVWRQAPGQG |
| | vH | LEWMGGFIPISGTVNYAQKFQGRVTITADESTSTAYMELSSLRSED |
| | | TAVYYCARPLDWTEDIWGQGTLVTVSS |
| 543 | RAS/P53 | EVQLVQSGAEVKKPGASVKVSCKASGYTFTAYYLHWLRQAPGQ |
| | vH | GLEWMGWMNTNNGATRYAQKFQDRVTMTRDTSINTAYMEMSG |
| | | LSSDDTAMYYCARGDISQDFADVWGQGTLVTVSS |
| 544 | RAS/P53 | GGTFSSYT |
| 545 | RAS/P53 | GYTFTAYY |
| 546 | RAS/P53 | FIPISGTV |
| 547 | RAS/P53 | MNTNNGAT |
| 548 | RAS/P53 | ARPLDWTEDI |
| 549 | RAS/P53 | ARGDISQDFADV |
| 550 | RAS/P53 | RASQDISKYLN |
| 551 | RAS/P53 | SRLHSGV |
| 552 | RAS/P53 | GNTLPYTFG |
| 553 | RAS/P53 | DYGVS |
| 554 | RAS/P53 | VIWGSETTYYNSALKS |
| 555 | RAS/P53 | YAMDYWG |
| 556 | RAS/P53 | EVKLQESGPGLVAPSQSLSVTCTVSGVSLPDYGVSWIQPPRKGLE |
| | | WLGVIWGSETTYYNSALKSRLTIIKDNSKSQVFLKMNSLQTDDTA |
| | | IYYCAKHYYYGGSYAMDYWGQGTSVTVSS |
| 557 | RAS/P53 | DIQMTQTTSSLSASLGDRVTISCRASQDISKYLNWYQQKPDGTVK |
| | | LLIYHTSRLHSGVPSRFSGSGSGTDYSLTISNLEQEDIATYFCQQGN |
| | | TLPYTFGGGTKLEIT |
| 558 | RAS/P53 | DIQMTQTTSSLSASLGDRVTISCRASQDISKYLNWYQQKPDGTVK |
| | | LLIYHTSRLHSGVPSRFSGSGSGTDYSLTISNLEQEDIATYFCQQGN |
| | | TLPYTFGGGTKLEITGSTSGSGKPGSGEGSTKGEVKLQESGPGLVA |
| | | PSQSLSVTCTVSGVSLPDYGVSWIRQPPRKGLEWLGVIWGSETTY |
| | | YNSALKSRLTIIKDNSKSQVFLKMNSLQTDDTAIYYCAKHYYYGG |
| | | SYAMDYWGQGTSVTVSS |
| 559 | RAS/P53 | KASQNVGTNVA |
| 560 | RAS/P53RAS/ | SATYRNS |
| | P53 | |
| 561 | RAS/P53 | QQYNRYPYT |
| 562 | RAS/P53 | SYWMN |
| 563 | RAS/P53 | QIYPGDGDTNYNGKFKG |
| 564 | RAS/P53 | KTISSVVDFYFDY |
| 565 | RAS/P53 | EVKLQQSGAELVRPGSSVKISCKASGYAFSSYWMNWVKQRPGQG |
| | | LEWIGQIYPGDGDTNYNGKFKGQATLTADKSSSTAYMQLSGLTSE |
| | | DSAVYFCARKTISSVVDFYFDYWGQGTTVTVSS |
| 566 | RAS/P53 | DIELTQSPKFMSTSVGDRVSVTCKASQNVGTNVAWYQQKPGQSP |
| | | KPLIYSATYRNSGVPDRFTGSGSGTDFTLTITNVQSKDLADYFCQQ |
| | | YNRYPYTSGGGTKLEIKR |
| 567 | RAS/P53 | EVKLQQSGAELVRPGSSVKISCKASGYAFSSYWMNWVKQRPGQG |
| | | LEWIGQIYPGDGDTNYNGKFKGQATLTADKSSSTAYMQLSGLTSE |
| | | DSAVYFCARKTISSVVDFYFDYWGQGTTVTVSSGGGGSGGGGSG |
| | | GGGSDIELTQSPKFMSTSVGDRVSVTCKASQNVGTNVAWYQQKP |
| | | GQSPKPLIYSATYRNSGVPDRFTGSGSGTDFTLTITNVQSKDLADY |
| | | FCQQYNRYPYTSGGGTKLEIKR |
| 568 | RAS/P53 | HYYYGGSYAMDY |
| 569 | RAS/P53 | HTSRLHS |
| 570 | RAS/P53 | QQGNTLPYT |
| 571 | RAS/P53 | gacatccagatgacccagaccacctccagcctgagcgccagcctgggcgaccgggtgaccatcagct |
| | | gccgggccagccaggacatcagcaagtacctgaactggtatcagcagaagcccgacggcaccgtcaa |
| | | gctgctgatctaccacaccagccggctgcacagcggcgtgcccagccggtttagcggcagcggctccg |
| | | gcaccgactacagcctgaccatctccaacctggaacaggaagatatcgccacctacttttgccagcagg |
| | | gcaacacactgccctacacctttggggggaacaaagctggaaatcaccggcagcacctccggcag |
| | | cggcaagcctggcagcggcgagggcagcaccaagggcgaggtgaagctgcaggaaagcggccctg |
| | | gcctggtggcccccagccagagcctgagcgtgacctgcaccgtgagcggcgtgagcctgcccgacta |
| | | cggcgtgagctggatccggcagccccccaggaagggcctggaatggctgggcgtgatctggggcag |
| | | cgagaccacctactacaacagcgccctgaagagccggctgaccatcatcaaggacaacagcaagagc |
| | | caggtgttcctgaagatgaacagcctgcagaccgacgacaccgccatctactactgcgccaagcactac |
| | | tactacggcggcagctacgccatggactactggggccagggcaccagcgtgaccgtgagcagc |
| 572 | PIK3CA | DRGSQS |
| 573 | PIK3CA | IYSNGD |
| 574 | PIK3CA | CAGNTGTASKLTF |
| 575 | PIK3CA | SGDLS |
| 576 | PIK3CA | YYNGEE |
| 577 | PIK3CA | CASSGLAGGPVSGANVLTF |
| 578 | PIK3CA | MKSLRVLLVILWLQLSWVWSQQKEVEQNSGPLSVPEGAIASLNCT |
| | | YSDRGSQSFFWYRQYSGKSPELIMSIYSNGDKEDGRFTAQLNKAS |
| | | QYVSLLIRDSQPSDSATYLCAGNTGTASKLTFGTGTRLQVTL |
| 579 | PIK3CA | MKSLRVLLVILWLQLSWVWSQQKEVEQNSGPLSVPEGAIASLNCT |
| | | YSDRGSQSFFWYRQYSGKSPELIMSIYSNGDKEDGRFTAQLNKAS |
| | | QYVSLLIRDSQPSDSATYLCAGNTGTASKLTFGTGTRLQVTLNIQN |
| | | PEPAVYQLKDPRSQDSTLCLFTDFDSQINVPKTMESGTFITDKCVL |
| | | DMKAMDSKSNGAIAWSNQTSFTCQDIFKETNATYPSSDVPCDATL |
| | | TEKSFETDMNLNFQNLLVIVLRILLLKVAGENLLMTLRLWSS |
| 580 | PIK3CA | MGFRLLCCVAFCLLGAGPVDSGVTQTPKHLITATGQRVTLRCSPR |
| | | SGDLSVYWYQQSLDQGLQFLIQYYNGEERAKGNILERFSAQQFPD |
| | | LHSELNLSSLELGDSALYFCASSGLAGGPVSGANVLTFGAGSRLT |
| | | VL |
| 581 | PIK3CA | MGFRLLCCVAFCLLGAGPVDSGVTQTPKHLITATGQRVTLRCSPR |
| | | SGDLSVYWYQQSLDQGLQFLIQYYNGEERAKGNILERFSAQQFPD |
| | | LHSELNLSSLELGDSALYFCASSGLAGGPVSGANVLTFGAGSRLT |
| | | VLEDLRNVTPPKVSLFEPSKAEIANKQKATLVCLARGFFPDHVELS |
| | | WWVNGKEVHSGVCTDPQAYKESNYSYCLSSRLRVSATFWHNPR |
| | | NHFRCQVQFHGLSEEDKWPEGSPKPVTQNISAEAWGRADCGITSA |
| | | SYQQGVLSATILYEILLGKATLYAVLVSTLVVMAMVKRKNS |
| 582 | PIK3CA | NIATNDY |
| 583 | PIK3CA | GYKTK |
| 584 | PIK3CA | CLVGGAYTGGFKTIF |
| 585 | PIK3CA | SGHAT |
| 586 | PIK3CA | FQNNGV |
| 587 | PIK3CA | CASSLVAETYEQYF |
| 588 | PIK3CA | MRQVARVIVFLTLSTLSLAKTTQPISMDSYEGQEVNITCSHNNIAT |
| | | NDYITWYQQFPSQGPRFIIQGYKTKVTNEVASLFIPADRKSSTLSLP |
| | | RVSLSDTAVYYCLVGGAYTGGFKTIFGAGTRLFVKA |
| 589 | PIK3CA | MRQVARVIVFLTLSTLSLAKTTQPISMDSYEGQEVNITCSHNNIAT |
| | | NDYITWYQQFPSQGPRFIIQGYKTKVTNEVASLFIPADRKSSTLSLP |
| | | RVSLSDTAVYYCLVGGAYTGGFKTIFGAGTRLFVKANIQNPEPAV |
| | | YQLKDPRSQDSTLCLFTDFDSQINVPKTMESGTFITDKCVLDMKA |
| | | MDSKSNGAIAWSNQTSFTCQDIFKETNATYPSSDVPCDATLTEKSF |
| | | ETDMNLNFQNLLVIVLRILLLKVAGFNLLMTLRLWSS |
| 590 | PIK3CA | MGTRLLCWAALCLLGAELTEAGVAQSPRYKIIEKRQSVAFWCNPI |
| | | SGHATLYWYQQILGQGPKLLIQFQNNGVVDDSQLPKDRFSAERL |
| | | KGVDSTLKIQPAKLEDSAVYLCASSLVAETYEQYFGPGTRLTVT |
| 591 | PIK3CA | MGTRLLCWAALCLLGAELTEAGVAQSPRYKIIEKRQSVAFWCNPI |
| | | SGHATLYWYQQILGQGPKLLIQFQNNGVVDDSQLPKDRFSAERL |
| | | KGVDSTLKIQPAKLEDSAVYLCASSLVAETYEQYFGPGTRLTVTE |
| | | DLRNVTPPKVSLFEPSKAEIANKQKATLVCLARGFFPDHVELSWW |
| | | VNGKEVHSGVCTDPQAYKESNYSYCLSSRLRVSATFWHNPRNHF |
| | | RCQVQFHGLSEEDKWPEGSPKPVTQNISAEAWGRADCGITSASYQ |
| | | QGVLSATILYEILLGKATLYAVLVSTLVVMAMVKRKNS |
| 592 | PIK3CA | ATGYPS |
| 593 | PIK3CA | ATKADDK |
| 594 | PIK3CA | CALTVGGSYIPTF |
| 595 | PIK3CA | MGHRA |
| 596 | PIK3CA | YSYEKL |
| 597 | PIK3CA | CASSQGGQGWRETQYF |
| 598 | PIK3CA | MNYSPGLVSLILLLLGRTRGDSVTQMEGPVTLSEEAFLTINCTYTA |
| | | TGYPSLFWYVQYPGEGLQLLLKATKADDKGSNKGFEATYRKETT |
| | | SFHLEKGSVQVSDSAVYFCALTVGGSYIPTFGRGTSLIVHP |
| 599 | PIK3CA | MNYSPGLVSLILLLLGRTRGDSVTQMEGPVTLSEEAFLTINCTYTA |
| | | TGYPSLFWYVQYPGEGLQLLLKATKADDKGSNKGFEATYRKETT |
| | | SFHLEKGSVQVSDSAVYFCALTVGGSYIPTFGRGTSLIVHPNIQNP |
| | | EPAVYQLKDPRSQDSTLCLFTDFDSQINVPKTMESGTFITDKCVLD |
| | | MKAMDSKSNGAIAWSNQTSFTCQDIFKETNATYPSSDVPCDATLT |
| | | EKSFETDMNLNFQNLLVIVLRILLLKVAGFNLLMTLRLWSS |
| 600 | PIK3CA | MGCRLLCCAVLCLLGAVPIDTEVTQTPKHLVMGMTNKKSLKCEQ |
| | | HMGHRAMYWYKQKAKKPPELMFVYSYEKLSINESVPSRFSPECP |
| | | NSSLLNLHLHALQPEDSALYLCASSQGGQGWRETQYFGPGTRLL |
| | | VL |
| 601 | PIK3CA | MGCRLLCCAVLCLLGAVPIDTEVTQTPKHLVMGMTNKKSLKCEQ |
| | | HMGHRAMYWYKQKAKKPPELMFVYSYEKLSINESVPSRFSPECP |
| | | NSSLLNLHLHALQPEDSALYLCASSQGGQGWRETQYFGPGTRLL |
| | | VLEDLRNVTPPKVSLFEPSKAEIANKQKATLVCLARGFFPDHVELS |
| | | WWVNGKEVHSGVCTDPQAYKESNYSYCLSSRLRVSATFWHNPR |
| | | NHFRCQVQFHGLSEEDKWPEGSPKPVTQNISAEAWGRADCGITSA |
| | | SYQQGVLSATILYEILLGKATLYAVLVSTLVVMAMVKRKNS |
| 602 | PIK3CA | NIQNPDPAVYQLRDSKSSDKSVCLFTDFDSQTNVSQSKDSDVYIT |
| | | DKTVLDMRSMDFKSNSAVAWSNKSDFACANAFNNSIIPEDTFFPS |
| | | PESSCDVKLVEKSFETDTNLNFQNLSVIGFRILLLKVAGFNLLMTL |
| | | RLWSS |
| 603 | PIK3CA | NIQNPEPAVYQLKDPRSQDSTLCLFTDFDSQINVPKTMESGTFITD |
| | | KCVLDMKAMDSKSNGAIAWSNQTSFTCQDIFKETNATYPSSDVP |
| | | CDATLTEKSFETDMNLNFQNLLVIVLRILLLKVAGFNLLMTLRLW |
| | | SS |
| 604 | PIK3CA | EDLNKVFPPEVAVFEPSEAEISHTQKATLVCLATGFFPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSVSYQQGVLSATILYEILLGKATLYAVLVSALVLMAMVKRKDF |
| 605 | PIK3CA | EDLRNVTPPKVSLFEPSKAEIANKQKATLVCLARGFFPDHVELSW |
| | | WVNGKEVHSGVCTDPQAYKESNYSYCLSSRLRVSATFWHNPRN |
| | | HFRCQVQFHGLSEEDKWPEGSPKPVTQNISAEAWGRADCGITSAS |
| | | YQQGVLSATILYEILLGKATLYAVLVSTLVVMAMVKRKNS |
| 606 | PIK3CA | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 607 | RAS | CAVSVGPGNTGKLIF |
| 608 | RAS | CASSLGVLGLRYF |
| 609 | RAS | CAENSGGSNYKLTF |
| 610 | RAS | CASSWERAGKAFF |
| 611 | RAS | CAMSVFIYSTFIF |
| 612 | RAS | CASSGRQETQYF |
| 613 | RAS | CATDAYTRQLTF |
| 614 | RAS | CASGGPGANRPQHF |
| 615 | RAS | CAYRSLWGSGYALNF |
| 616 | RAS | CSVEKGAQETQYF |
| 617 | RAS | CAALFGNEKLTF |
| 618 | RAS | CATLQGWSYNEQFF |
| 619 | RAS | CALSTGGFKTIF |
| 620 | RAS | CSVLGPGTGGRGANYGYTF |
| 621 | RAS | CATTIDGQKLLF |
| 622 | RAS | CSASDRGSGELFF |
| 623 | RAS | CATDAYNARLMF |
| 624 | RAS | CSVVASGSVDTQYF |
| 625 | RAS | CAGPAGAQKLVF |
| 626 | RAS | CASSPVPYSGNTIYF |
| 627 | RAS | CAVSKSDKIIF |
| 628 | RAS | CATSEGGSTGTEAFF |
| 629 | RAS | CALNRNTGNQFYF |
| 630 | RAS | CASSIPQGNGYTF |
| 631 | RAS | MLLLLVPVLEVIFTLGGTRAQSVTQLGSHVSVSEGALVLLRCNYS |
| | | SSVPPYLFWYVQYPNQGLQLLLKYTSAATLVKGINGFEAEFKKSE |
| | | TSFHLTKPSAHMSDAAEYFCAVSVGPGNTGKLIFGQGTTLQVKPD |
| | | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 632 | RAS | MGIRLLCRVAFCFLAVGLVDVKVTQSSRYLVKRTGEKVFLECVQ |
| | | DMDHENMFWYRQDPGLGLRLIYFSYDVKMKEKGDIPEGYSVSRE |
| | | KKERFSLILESASTNQTSMYLCASSLGVLGLRYFGPGTRLTVTEDL |
| | | NKVFPPEVAVFEPSEAEISHTQKATLVCLATGFFPDHVELSWWVN |
| | | GKEVHSGVSTDPQPLKEQPALNDSRYCLSSRLRVSATFWQNPRNH |
| | | FRCQVQFYGLSENDEWTQDRAKPVTQIVSAEAWGRADCGFTSVS |
| | | YQQGVLSATILYEILLGKATLYAVLVSALVLMAMVKRKDF |
| 633 | RAS | MAGIRALFMYLWLQLDWVSRGESVGLHLPTLSVQEGDNSIINCA |
| | | YSNSASDYFIWYKQESGKGPQFIIDIRSNMDKRQGQRVTVLLNKT |
| | | VKHLSLQIAATQPGDSAVYFCAENSGGSNYKLTFGKGTLLTVNPN |
| | | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 634 | RAS | MGPGLLCWVLLCLLGAGSVETGVTQSPTHLIKTRGQQVTLRCSSQ |
| | | SGHNTVSWYQQALGQGPQFIFQYYREEENGRGNFPPRFSGLQFPN |
| | | YSSELNVNALELDDSALYLCASSWERAGKAFFGQGTRLTVVEDL |
| | | NKVFPPEVAVFEPSEAEISHTQKATLVCLATGFFPDHVELSWWVN |
| | | GKEVHSGVSTDPQPLKEQPALNDSRYCLSSRLRVSATFWQNPRNH |
| | | FRCQVQFYGLSENDEWTQDRAKPVTQIVSAEAWGRADCGFTSVS |
| | | YQQGVLSATILYEILLGKATLYAVLVSALVLMAMVKRKDF |
| 635 | RAS | MMKSLRVLLVILWLQLSWVWSQQKEVEQDPGPLSVPEGAIVSLN |
| | | CTYSNSAFQYFMWYRQYSRKGPELLMYTYSSGNKEDGRFTAQV |
| | | DKSSKYISLFIRDSQPSDSATYLCAMSVFIYSTFIFGSGTRLSVKPDI |
| | | QNPDPAVYQLRDSKSSDKSVCLFTDFDSQTNVSQSKDSDVYITDK |
| | | TVLDMRSMDFKSNSAVAWSNKSDFACANAFNNSIIPEDTFFPSPES |
| | | SCDVKLVEKSFETDTNLNFQNLSVIGFRILLLKVAGFNLLMTLRL |
| | | WSS |
| 636 | RAS | MGIRLLCRVAFCFLAVGLVDVKVTQSSRYLVKRTGEKVFLECVQ |
| | | DMDHENMFWYRQDPGLGLRLIYFSYDVKMKEKGDIPEGYSVSRE |
| | | KKERFSLILESASTNQTSMYLCASSGRQETQYFGPGTRLLVLEDLN |
| | | KVFPPEVAVFEPSEAEISHTQKATLVCLATGFFPDHVELSWWVNG |
| | | KEVHSGVSTDPQPLKEQPALNDSRYCLSSRLRVSATFWQNPRNHF |
| | | RCQVQFYGLSENDEWTQDRAKPVTQIVSAEAWGRADCGFTSVSY |
| | | QQGVLSATILYEILLGKATLYAVLVSALVLMAMVKRKDF |
| 637 | RAS | METLLGVSLVILWLQLARVNSQQGEEDPQALSIQEGENATMNCSY |
| | | KTSINNLQWYRQNSGRGLVHLILIRSNEREKHSGRLRVTLDTSKKS |
| | | SSLLITASRAADTASYFCATDAYTRQLTFGSGTQLTVLPDIQNPDP |
| | | AVYQLRDSKSSDKSVCLFTDFDSQTNVSQSKDSDVYITDKTVLDM |
| | | RSMDFKSNSAVAWSNKSDFACANAFNNSIIPEDTFFPSPESSCDVK |
| | | LVEKSFETDTNLNFQNLSVIGFRILLLKVAGFNLLMTLRLWSS |
| 638 | RAS | MSNQVLCCVVLCLLGANTVDGGITQSPKYLFRKEGQNVTLSCEQ |
| | | NLNHDAMYWYRQDPGQGLRLIYYSQIVNDFQKGDIAEGYSVSRE |
| | | KKESFPLTVTSAQKNPTAFYLCASGGPGANRPQHFGDGTRLSILED |
| | | LNKVFPPEVAVFEPSEAEISHTQKATLVCLATGFFPDHVELSWWV |
| | | NGKEVHSGVSTDPQPLKEQPALNDSRYCLSSRLRVSATFWQNPRN |
| | | HFRCQVQFYGLSENDEWTQDRAKPVTQIVSAEAWGRADCGFTSV |
| | | SYQQGVLSATILYEILLGKATLYAVLVSALVLMAMVKRKDF |
| 639 | RAS | MACPGFLWALVISTCLEFSMAQTVTQSQPEMSVQEAETVTLSCTY |
| | | DTSESDYYLFWYKQPPSRQMILVIRQEAYKQQNATENRFSVNFQK |
| | | AAKSFSLKISDSQLGDAAMYFCAYRSLWGSGYALNFGKGTSLLV |
| | | TPHIQNPDPAVYQLRDSKSSDKSVCLFTDFDSQTNVSQSKDSDVYI |
| | | TDKTVLDMRSMDFKSNSAVAWSNKSDFACANAFNNSIIPEDTFFP |
| | | SPESSCDVKLVEKSFETDTNLNFQNLSVIGFRILLLKVAGFNLLMT |
| | | LRLWSS |
| 640 | RAS | MLSLLLLLLGLGSVFSAVISQKPSRDICQRGTSLTIQCQVDSQVTM |
| | | MFWYRQQPGQSLTLIATANQGSEATYESGFVIDKFPISRPNLTFST |
| | | LTVSNMSPEDSSIYLCSVEKGAQETQYFGPGTRLLVLEDLNKVFPP |
| | | EVAVFEPSEAEISHTQKATLVCLATGFFPDHVELSWWVNGKEVHS |
| | | GVSTDPQPLKEQPALNDSRYCLSSRLRVSATFWQNPRNHFRCQVQ |
| | | FYGLSENDEWTQDRAKPVTQIVSAEAWGRADCGFTSVSYQQGVL |
| | | SATILYEILLGKATLYAVLVSALVLMAMVKRKDF |
| 641 | RAS | MTSIRAVFIFLWLQLDLVNGENVEQHPSTLSVQEGDSAVIKCTYS |
| | | DSASNYFPWYKQELGKGPQLIIDIRSNVGEKKDQRIAVTLNKTAK |
| | | HFSLHITETQPEDSAVYFCAALFGNEKLTFGTGTRLTIIPNIQNPDP |
| | | AVYQLRDSKSSDKSVCLFTDFDSQTNVSQSKDSDVYITDKTVLDM |
| | | RSMDFKSNSAVAWSNKSDFACANAFNNSIIPEDTFFPSPESSCDVK |
| | | LVEKSFETDTNLNFQNLSVIGFRILLLKVAGFNLLMTLRLWSS |
| 642 | RAS | MGTRLLFWVAFCLLGADHTGAGVSQSPSNKVTEKGKDVELRCDP |
| | | ISGHTALYWYRQSLGQGLEFLIYFQGNSAPDKSGLPSDRFSAERTG |
| | | GSVSTLTIQRTQQEDSAVYLCATLQGWSYNEQFFGPGTRLTVLED |
| | | LNKVFPPEVAVFEPSEAEISHTQKATLVCLATGFFPDHVELSWWV |
| | | NGKEVHSGVSTDPQPLKEQPALNDSRYCLSSRLRVSATFWQNPRN |
| | | HFRCQVQFYGLSENDEWTQDRAKPVTQIVSAEAWGRADCGFTSV |
| | | SYQQGVLSATILYEILLGKATLYAVLVSALVLMAMVKRKDF |
| 643 | RAS | MKPTLISVLVIIFILRGTRAQRVTQPEKLLSVFKGAPVELKCNYSYS |
| | | GSPELFWYVQYSRQRLQLLLRHISRESIKGFTADLNKGETSFHLKK |
| | | PFAQEEDSAMYYCALSTGGFKTIFGAGTRLFVKANIQNPDPAVYQ |
| | | LRDSKSSDKSVCLFTDFDSQTNVSQSKDSDVYITDKTVLDMRSMD |
| | | FKSNSAVAWSNKSDFACANAFNNSIIPEDTFFPSPESSCDVKLVEK |
| | | SFETDTNLNFQNLSVIGFRILLLKVAGFNLLMTLRLWSS |
| 644 | RAS | MLSLLLLLLGLGSVFSAVISQKPSRDICQRGTSLTIQCQVDSQVTM |
| | | MFWYRQQPGQSLTLIATANQGSEATYESGFVIDKFPISRPNLTFST |
| | | LTVSNMSPEDSSIYLCSVLGPGTGGRGANYGYTFGSGTRLTVVED |
| | | LNKVFPPEVAVFEPSEAEISHTQKATLVCLATGFFPDHVELSWWV |
| | | NGKEVHSGVSTDPQPLKEQPALNDSRYCLSSRLRVSATFWQNPRN |
| | | HFRCQVQFYGLSENDEWTQDRAKPVTQIVSAEAWGRADCGFTSV |
| | | SYQQGVLSATILYEILLGKATLYAVLVSALVLMAMVKRKDF |
| 645 | RAS | METLLGVSLVILWLQLARVNSQQGEEDPQALSIQEGENATMNCSY |
| | | KTSINNLQWYRQNSGRGLVHLILIRSNEREKHSGRLRVTLDTSKKS |
| | | SSLLITASRAADTASYFCATTIDGQKLLFARGTMLKVDLNIQNPDP |
| | | AVYQLRDSKSSDKSVCLFTDFDSQTNVSQSKDSDVYITDKTVLDM |
| | | RSMDFKSNSAVAWSNKSDFACANAFNNSIIPEDTFFPSPESSCDVK |
| | | LVEKSFETDTNLNFQNLSVIGFRILLLKVAGFNLLMTLRLWSS |
| 646 | RAS | MLLLLLLLGPAGSGLGAVVSQHPSRVICKSGTSVKIECRSLDFQAT |
| | | TMFWYRQFPKKSLMLMATSNEGSKATYEQGVEKDKFLINHASLT |
| | | LSTLTVTSAHPEDSSFYICSASDRGSGELFFGEGSRLTVLEDLNKVF |
| | | PPEVAVFEPSEAEISHTQKATLVCLATGFFPDHVELSWWVNGKEV |
| | | HSGVSTDPQPLKEQPALNDSRYCLSSRLRVSATFWQNPRNHFRCQ |
| | | VQFYGLSENDEWTQDRAKPVTQIVSAEAWGRADCGFTSVSYQQG |
| | | VLSATILYEILLGKATLYAVLVSALVLMAMVKRKDF |
| 647 | RAS | METLLGVSLVILWLQLARVNSQQGEEDPQALSIQEGENATMNCSY |
| | | KTSINNLQWYRQNSGRGLVHLILIRSNEREKHSGRLRVTLDTSKKS |
| | | SSLLITASRAADTASYFCATDAYNARLMFGDGTQLVVKPNIQNPD |
| | | PAVYQLRDSKSSDKSVCLFTDFDSQTNVSQSKDSDVYITDKTVLD |
| | | MRSMDFKSNSAVAWSNKSDFACANAFNNSIIPEDTFFPSPESSCDV |
| | | KLVEKSFETDTNLNFQNLSVIGFRILLLKVAGFNLLMTLRLWSS |
| 648 | RAS | MLSLLLLLLGLGSVFSAVISQKPSRDICQRGTSLTIQCQVDSQVTM |
| | | MFWYRQQPGQSLTLIATANQGSEATYESGFVIDKFPISRPNLTFST |
| | | LTVSNMSPEDSSIYLCSVVASGSVDTQYFGPGTRLTVLEDLNKVFP |
| | | PEVAVFEPSEAEISHTQKATLVCLATGFFPDHVELSWWVNGKEVH |
| | | SGVSTDPQPLKEQPALNDSRYCLSSRLRVSATFWQNPRNHFRCQV |
| | | QFYGLSENDEWTQDRAKPVTQIVSAEAWGRADCGFTSVSYQQGV |
| | | LSATILYEILLGKATLYAVLVSALVLMAMVKRKDF |
| 649 | RAS | MVLKFSVSILWIQLAWVSTQLLEQSPQFLSIQEGENLTVYCNSSSV |
| | | FSSLQWYRQEPGALEGPVLLVTVVTGGEVKKLKRLTFQFGDARK |
| | | DSSLHITAAQPGDTGLYLCAGPAGAQKLVFGQGTRLTINPNIQNP |
| | | DPAVYQLRDSKSSDKSVCLFTDFDSQTNVSQSKDSDVYITDKTVL |
| | | DMRSMDFKSNSAVAWSNKSDFACANAFNNSIIPEDTFFPSPESSCD |
| | | VKLVEKSFETDTNLNFQNLSVIGFRILLLKVAGFNLLMTLRLWSS |
| 650 | RAS | MSISLLCCAAFPLLWAGPVNAGVTQTPKFRLKIGQSMTLQCTQD |
| | | MNHNYMYWYRQDPGMGLKLIYYSVGAGITDKGEVPNGYNVSRS |
| | | TTEDFPLRLELAAPSQTSVYFCASSPVPYSGNTIYFGEGSWLTVVE |
| | | DLNKVFPPEVAVFEPSEAEISHTQKATLVCLATGFFPDHVELSWW |
| | | VNGKEVHSGVSTDPQPLKEQPALNDSRYCLSSRLRVSATFWQNPR |
| | | NHFRCQVQFYGLSENDEWTQDRAKPVTQIVSAEAWGRADCGFTS |
| | | VSYQQGVLSATILYEILLGKATLYAVLVSALVLMAMVKRKDF |
| 651 | RAS | MLLLLVPVLEVIFTLGGTRAQSVTQLGSHVSVSEGALVLLRCNYS |
| | | SSVPPYLFWYVQYPNQGLQLLLKYTSAATLVKGINGFEAEFKKSE |
| | | TSFHLTKPSAHMSDAAEYFCAVSKSDKIIFGKHILPNIQNPDPAVY |
| | | QLRDSKSSDKSVCLFTDFDSQTNVSQSKDSDVYITDKTVLDMRSM |
| | | DFKSNSAVAWSNKSDFACANAFNNSIIPEDTFFPSPESSCDVKLVE |
| | | KSFETDTNLNFQNLSVIGFRILLLKVAGFNLLMTLRLWSS |
| 652 | RAS | MASLLFFCGAFYLLGTGSMDADVTQTPRNRITKTGKRIMLECSQT |
| | | KGHDRMYWYRQDPGLGLRLIYYSFDVKDINKGEISDGYSVSRQA |
| | | QAKFSLSLESAIPNQTALYFCATSEGGSTGTEAFFGQGTRLTVVED |
| | | LNKVFPPEVAVFEPSEAEISHTQKATLVCLATGFFPDHVELSWWV |
| | | NGKEVHSGVSTDPQPLKEQPALNDSRYCLSSRLRVSATFWQNPRN |
| | | HFRCQVQFYGLSENDEWTQDRAKPVTQIVSAEAWGRADCGFTSV |
| | | SYQQGVLSATILYEILLGKATLYAVLVSALVLMAMVKRKDF |
| 653 | RAS | MNYSPGLVSLILLLLGRTRGNSVTQMEGPVTLSEEAFLTINCTYTA |
| | | TGYPSLFWYVQYPGEGLQLLLKATKADDKGSNKGFEATYRKETT |
| | | SFHLEKGSVQVSDSAVYFCALNRNTGNQFYFGTGTSLTVIPNIQNP |
| | | DPAVYQLRDSKSSDKSVCLFTDFDSQTNVSQSKDSDVYITDKTVL |
| | | DMRSMDFKSNSAVAWSNKSDFACANAFNNSIIPEDTFFPSPESSCD |
| | | VKLVEKSFETDTNLNFQNLSVIGFRILLLKVAGFNLLMTLRLWSS |
| 654 | RAS | MSNQVLCCVVLCLLGANTVDGGITQSPKYLFRKEGQNVTLSCEQ |
| | | NLNHDAMYWYRQDPGQGLRLIYYSQIVNDFQKGDIAEGYSVSRE |
| | | KKESFPLTVTSAQKNPTAFYLCASSIPQGNGYTFGSGTRLTVVEDL |
| | | NKVFPPEVAVFEPSEAEISHTQKATLVCLATGFFPDHVELSWWVN |
| | | GKEVHSGVSTDPQPLKEQPALNDSRYCLSSRLRVSATFWQNPRNH |
| | | FRCQVQFYGLSENDEWTQDRAKPVTQIVSAEAWGRADCGFTSVS |
| | | YQQGVLSATILYEILLGKATLYAVLVSALVLMAMVKRKDF |
| 655 | RAS | atgctcctgctgctcgtcccagtgctcgaggtgatttttaccctgggaggaaccagagcccagtcggtga |
| | | cccagcttggcagccacgtctctgtctctgaaggagccctggttctgctgaggtgcaactactcatcgtct |
| | | gttccaccatatctcttctggtatgtgcaataccccaaccaaggactccagcttctcctgaagtacacatca |
| | | gcggccaccctggttaaaggcatcaacggttttgaggctgaatttaagaagagtgaaacctccttccacct |
| | | gacgaaaccctcagcccatatgagcgacgcggctgagtacttctgtgctgtgagtgtaggtcctggcaac |
| | | acaggcaaactaatctttgggcaagggacaactttacaagtaaaaccagatatccagaaccctgaccctg |
| | | ccgtgtaccagctgagagactctaaatccagtgacaagtctgtctgcctattcaccgattttgattctcaaac |
| | | aaatgtgtcacaaagtaaggattctgatgtgtatatcacagacaaaactgtgctagacatgaggtctatgga |
| | | cttcaagagcaacagtgctgtggcctggagcaacaaatctgactttgcatgtgcaaacgccttcaacaac |
| | | agcattattccagaagacaccttcttccccagcccagaaagttcctgtgatgtcaagctggtcgagaaaag |
| | | ctttgaaacagatacgaacctaaactttcaaaacctgtcagtgattgggttccgaatcctcctcctgaaagg |
| | | tggccgggtttaatctgctcatgacgctgcggctgtggtccagcTAA |
| 656 | RAS | atgggaatcaggctcctgtgtcgtgtggccttttgtttcctggctgtaggcctcgtagatgtgaaagtaacc |
| | | cagagctcgagatatctagtcaaaaggacgggagagaaagtttttctggaatgtgtccaggatatggacc |
| | | atgaaaatatgttctggtatcgacaagacccaggtctggggctacggctgatctatttctcatatgatgttaa |
| | | aatgaaagaaaaaggagatattcctgaggggtacagtgtctctagagagaagaaggagcgcttctccct |
| | | gattctggagtccgccagcaccaaccagacatctatgtacctctgtgccagcagtttgggggttctaggcc |
| | | tgcggtacttcgggccgggcaccaggctcacggtcacagaggacctgaacaaggtgttcccacccgag |
| | | gtcgctgtgtttgagccatcagaagcagagatctcccacacccaaaaggccacactggtgtgcctggcc |
| | | acaggcttcttccctgaccacgtggagctgagctggtgggtgaatgggaaggaggtgcacagtggggt |
| | | cagcacggacccgcagcccctcaaggagcagcccgccctcaatgactccagatactgcctgagcagc |
| | | cgcctgagggtctcggccaccttctggcagaacccccgcaaccacttccgctgtcaagtccagttctacg |
| | | ggctctcggagaatgacgagtggacccaggatagggccaaacccgtcacccagatcgtcagcgccga |
| | | ggcctggggtagagcagactgtggctttacctcggtgtcctaccagcaaggggtcctgtctgccaccatc |
| | | ctctatgagatcctgctagggaaggccaccctgtatgctgtgctggtcagcgcccttgtgttgatggccat |
| | | ggtcaagagaaaggatttcTAA |
| 657 | RAS | atggcaggcattcgagctttatttatgtacttgtggctgcagctggactgggtgagcagaggagagagtgt |
| | | ggggctgcatcttcctaccctgagtgtccaggagggtgacaactctattatcaactgtgcttattcaaacag |
| | | cgcctcagactacttcatttggtacaagcaagaatctqqaaaaqqtcctcaattcattataqacattcqttca |
| | | aatatqqacaaaaqqcaaqqccaaagagtcaccgttttattgaataagacagtgaaacatctctctctgca |
| | | aattgcagctactcaacctggagactcagctgtctacttttgtgcagagaatagtggaggtagcaactataa |
| | | actgacatttggaaaaggaactctcttaaccgtgaatccaaatatccagaaccctgaccctgccgtgtacc |
| | | agctgagagactctaaatccagtgacaagtctgtctgcctattcaccgattttgattctcaaacaaatgtgtc |
| | | acaaagtaaggattctgatgtgtatatcacagacaaaactgtgctagacatgaggtctatggacttcaaga |
| | | gcaacagtgctgtggcctggagcaacaaatctgactttgcatgtgcaaacgccttcaacaacagcattatt |
| | | ccagaagacaccttcttccccagcccagaaagttcctgtgatgtcaagctggtcgagaaaagctttgaaa |
| | | cagatacgaacctaaactttcaaaacctgtcagtgattgggttccgaatcctcctcctgaaagtggccggg |
| | | tttaatctgctcatgacgctgcggctgtggtccagcTAA |
| 658 | RAS | atgggccctgggctcctctgctgggtgctgctttgtctcctgggagcaggctcagtggagactggagtca |
| | | cccaaagtcccacacacctgatcaaaacgagaggacagcaagtgactctgagatgctcttctcagtctgg |
| | | gcacaacactgtgtcctggtaccaacaggccctgggtcaggggccccagtttatctttcagtattataggg |
| | | aggaagagaatggcagaggaaacttccctcctagattctcaggtctccagttccctaattatagctctgag |
| | | ctgaatgtgaacgccttggagctggacgactcggccctgtatctctgtgccagcagctgggaaagggcg |
| | | gggaaagctttctttggacaaggcaccagactcacagttgtagaggacctgaacaaggtgttcccaccc |
| | | gaggtcgctgtgtttgagccatcagaagcagagatctcccacacccaaaaggccacactggtgtgcctg |
| | | gccacaggcttcttccctgaccacgtggagctgagctggtgggtgaatgggaaggaggtgcacagtgg |
| | | ggtcagcacggacccgcagcccctcaaggagcagcccgccctcaatgactccagatactgcctgagc |
| | | agccgcctgagggtctcggccaccttctggcagaacccccgcaaccacttccgctgtcaagtccagttct |
| | | acgggctctcggagaatgacgagtggacccaggatagggccaaacccgtcacccagatcgtcagcgc |
| | | cgaggcctggggtagagcagactgtggctttacctcggtgtcctaccagcaaggggtcctgtctgccac |
| | | catcctctatgagatcctgctagggaaggccaccctgtatgctgtgctggtcagcgcccttgtgttgatgg |
| | | ccatggtcaagagaaaggatttcTAA |
| 659 | RAS | atgatgaaatccttgagagttttactggtgatcctgtggcttcagttaagctgggtttggagccaacagaag |
| | | gaggtggagcaggatcctggaccactcagtgttccagagggagccattgtttctctcaactgcacttaca |
| | | gcaacagtgcttttcaatacttcatgtggtacagacagtattccagaaaaggccctgagttgctgatgtaca |
| | | catactccagtggtaacaaagaagatggaaggtttacagcacaggtcgataaatccagcaagtatatctc |
| | | cttgttcatcagagactcacagcccagtgattcagccacctacctctgtgcaatgagcgtctttatttatagc |
| | | acattcatctttgggagtgggacaagattatcagtaaaacctgatatccagaaccctgaccctgccgtgta |
| | | ccagctgagagactctaaatccagtgacaagtctgtctgcctattcaccgattttgattctcaaacaaatgtg |
| | | tcacaaagtaaggattctgatgtgtatatcacagacaaaactgtgctagacatgaggtctatggacttcaag |
| | | agcaacagtgctgtggcctggagcaacaaatctgactttgcatgtgcaaacgccttcaacaacagcattat |
| | | tccagaagacaccttcttccccagcccagaaagttcctgtgatgtcaagctggtcgagaaaagctttgaaa |
| | | cagatacgaacctaaactttcaaaacctgtcagtgattgggttccgaatcctcctcctgaaagtgqccggg |
| | | tttaatctgctcatgacgctgcggctgtggtccagcTAA |
| 660 | RAS | atgggaatcaggctcctgtgtcgtgtggccttttgtttcctggctgtaggcctcgtagatgtgaaagtaacc |
| | | cagagctcgagatatctagtcaaaaggacgggagagaaagtttttctggaatgtgtccaggatatggacc |
| | | atgaaaatatgttctggtatcgacaagacccaggtctggggctacggctgatctatttctcatatgatgttaa |
| | | aatgaaagaaaaaggagatattcctgaggggtacagtgtctctagagagaagaaggagcgcttctccct |
| | | gattctggagtccgccagcaccaaccagacatctatgtacctctgtgccagctctgggaggcaagagac |
| | | ccagtacttcgggccaggcacgcggctcctggtgctcgaggacctgaacaaggtgttcccacccgagg |
| | | tcgctgtgtttgagccatcagaagcagagatctcccacacccaaaaggccacactggtgtgcctggcca |
| | | caggcttcttccctgaccacgtggagctgagctggtgggtgaatgggaaggaggtgcacagtggggtc |
| | | agcacggacccgcagcccctcaaggagcagcccgccctcaatgactccagatactgcctgagcagcc |
| | | gcctgagggtctcggccaccttctggcagaacccccgcaaccacttccgctgtcaagtccagttctacgg |
| | | gctctcggagaatgacgagtggacccaggatagggccaaacccgtcacccagatcgtcagcgccgag |
| | | gcctggggtagagcagactgtggctttacctcggtgtcctaccagcaaggggtcctgtctgccaccatcc |
| | | tctatgagatcctgctagggaaggccaccctgtatgctgtgctggtcagcgcccttgtgttgatggccatg |
| | | gtcaagagaaaggatttcTAA |
| 661 | RAS | atggaaactctcctgggagtgtctttggtgattctatggcttcaactggctagggtgaacagtcaacaggg |
| | | agaagaggatcctcaggccttgagcatccaggagggtgaaaatgccaccatgaactgcagttacaaaa |
| | | ctagtataaacaatttacagtggtatagacaaaattcaggtagaggccttgtccacctaattttaatacgttc |
| | | aaatgaaagagagaaacacagtggaagattaagagtcacgcttgacacttccaagaaaagcagttccttgt |
| | | tgatcacggcttcccgggcagcagacactgcttcttacttctgtgctacggacgcgtacacaaggcaact |
| | | gacctttggatctgggacacaattgactgttttacctgatatccagaaccctgaccctgccgtgtacgacat |
| | | gaggtctatggacttcaagagcaacagtgctgtggcctggagcaacaaatctgactttgcatgtgcaaac |
| | | gccttcaacaacagcattattccagaagacaccttcttccccagcccagaaagttcctgtgatgtcaagct |
| | | ggtcgagaaaagctttgaaacagatacgaacctaaactttcaaaacctgtcagtgattgggttccgaatcc |
| | | tcctcctgaaagtggccgggtttaatctgctcatgacgctgcggctgtggtccagcTAA |
| 662 | RAS | atgagcaaccaggtgctctgctgtgtggtcctttgtctcctgggagcaaacaccgtggatggtggaatcac |
| | | tcagtccccaaagtacctgttcagaaaggaaggacagaatgtgaccctgagttgtgaacagaatttgaac |
| | | cacgatgccatgtactggtaccgacaggacccagggcaagggctgagattgatctactactcacagata |
| | | gtaaatgactttcagaaaggagatatagctgaagggtacagcgtctctcgggagaagaaggaatcctttc |
| | | ctctcactgtgacatcggcccaaaagaacccgacagctttctatctctgtgccagtgggggaccggggg |
| | | ctaacaggccccagcattttggtgatgggactcgactctccatcctagaggacctgaacaaggtgttccc |
| | | acccgaggtcgctgtgtttgagccatcagaagcagagatctcccacacccaaaaggccacactggtgtg |
| | | cctggccacaggcttcttccctgaccacgtggagctgagctggtgggtgaatgggaaggaggtgcaca |
| | | gtggggtcagcacggacccgcagcccctcaaggagcagcccgccctcaatgactccagatactgcctg |
| | | agcagccgcctgagggtctcggccaccttctggcagaacccccgcaaccacttccgctgtcaagtccag |
| | | ttctacgggctctcggagaatgacgagtggacccaggatagggccaaacccgtcacccagatcgtcag |
| | | cgccgaggcctggggtagagcagactgtggctttacctcggtgtcctaccagcaaggggtcctgtctgc |
| | | caccatcctctatgagatcctgctagggaaggccaccctgtatgctgtgctggtcagcgcccttgtgttgat |
| | | qqccatqqtcaaqaqaaaqqatttcTAA |
| 663 | RAS | atggcatgccctggcttcctgtgggcacttgtgatctccacctgtcttgaatttagcatggctcagacagtca |
| | | ctcagtctcaaccagagatgtctgtgcaggaggcagagaccgtgaccctgagctgcacatatgacacca |
| | | gtgagagtgattattatttattctggtacaagcagcctcccagcaggcagatgattctcgttattcgccaaga |
| | | agcttataagcaacagaatgcaacagagaatcgtttctctgtgaacttccagaaagcagccaaatccttca |
| | | gtctcaagatctcagactcacagctgggggatgccgcgatgtatttctgtgcttataggagcctttgggggt |
| | | ccgggtatgcactcaacttcggcaaaggcacctcgctgttggtcacaccccatatccagaaccctgaccc |
| | | tgccgtgtaccagctgagagactctaaatccagtgacaagtctgtctgcctattcaccgattttgattctcaa |
| | | acaaatgtgtcacaaagtaaggattctgatgtgtatatcacagacaaaactgtgctagacatgaggtctatg |
| | | gacttcaagagcaacagtgctgtggcctggagcaacaaatctgactttgcatgtgcaaacgccttcaaca |
| | | acagcattattccagaagacaccttcttccccagcccagaaagttcctgtgatgtcaagctggtcgagaaa |
| | | agctttgaaacagatacgaacctaaactttcaaaacctgtcagtgattgggttccgaatcctcctcctqaaa |
| | | qtggccgggtttaatctgctcatgacgctgcggctgtggtccaqcTAA |
| 664 | RAS | atgctgagtcttctgctccttctcctgggactaggctctgtgttcagtgctgtcatctctcaaaagccaagca |
| | | gggatatctgtcaacgtggaacctccctgacgatccagtgtcaagtcgatagccaagtcaccatgatgttc |
| | | tggtaccgtcagcaacctggacagagcctgacactgatcgcaactgcaaatcagggctctgaggccac |
| | | atatgagagtggatttgtcattgacaagtttcccatcagccgcccaaacctaacattctcaactctgactgtg |
| | | agcaacatgagccctgaagacagcagcatatatctctgcagcgttgaaaagggcgcgcaagagaccca |
| | | gtacttcgggccaggcacgcggctcctggtgctcgaggacctgaacaaggtgttcccacccgaggtcg |
| | | ctgtgtttgagccatcagaagcagagatctcccacacccaaaaggccacactggtgtgcctggccacag |
| | | gcttcttccctgaccacgtggagctgagctggtgggtgaatgggaaggaggtgcacagtggggtcagc |
| | | acggacccgcagcccctcaaggagcagcccgccctcaatgactccagatactgcctgagcagccgcct |
| | | gagggtctcggccaccttctggcagaacccccgcaaccacttccgctgtcaagtccagttctacgggctc |
| | | tcggagaatgacgagtggacccaggatagggccaaacccgtcacccagatcgtcagcgccgaggcct |
| | | ggggtagagcagactgtggctttacctcggtgtcctaccagcaaggggtcctgtctgccaccatcctctat |
| | | gagatcctgctagggaaggccaccctgtatgctgtgctggtcagcgcccttgtgttgatggccatggtca |
| | | agagaaaggatttcTAA |
| 665 | RAS | atgacatccattcgagctgtatttatattcctgtggctgcagctggacttggtgaatggagagaatgtggag |
| | | cagcatccttcaaccctgagtgtccaggagggagacagcgctgttatcaagtgtacttattcagacagtgc |
| | | ctcaaactacttcccttggtataagcaagaacttggaaaaggacctcagcttattatagacattcgttcaaat |
| | | gtgggcgaaaagaaagaccaacgaattgctgttacattgaacaagacagccaaacatttctccctgcaca |
| | | tcacagagacccaacctgaagactcggctgtctacttctgtgcagcactctttggaaatgagaaattaacct |
| | | ttgggactggaacaagactcaccatcatacccaatatccagaaccctgaccctgccgtgtaccagctgag |
| | | agactctaaatccagtgacaagtctgtctgcctattcaccgattttgattctcaaacaaatgtgtcacaaagt |
| | | aaggattctgatgtgtatatcacagacaaaactgtgctagacatgaggtctatggacttcaagagcaacag |
| | | tgctgtggcctggagcaacaaatctgactttgcatgtgcaaacgccttcaacaacagcattattccagaag |
| | | acaccttcttccccagcccagaaagttcctgtgatgtcaagctggtcgagaaaagctttgaaacagatacg |
| | | aacctaaactttcaaaacctgtcagtgattgggttccgaatcctcctcctgaaagtggccgggtttaatctgc |
| | | tcatgacgctgcggctgtggtccagcTAA |
| 666 | RAS | atgggcaccaggctcctcttctgggtggccttctgtctcctgggggcagatcacacaggagctggagtct |
| | | cccagtcccccagtaacaaggtcacagagaagggaaaggatgtagagctcaggtgtgatccaatttcag |
| | | gtcatactgccctttactggtaccgacagagcctggggcagggcctggagtttttaatttacttccaaggca |
| | | acagtgcaccagacaaatcagggctgcccagtgatcgcttctctgcagagaggactgggggatccgtct |
| | | ccactctgacgatccagcgcacacagcaggaggactcggccgtgtatctctgtgccaccctacagggtt |
| | | ggtcttataatgagcagttcttcgggccagggacacggctcaccgtgctagaggacctgaacaaggtgtt |
| | | cccacccgaggtcgctgtgtttgagccatcagaagcagagatctcccacacccaaaaggccacactggt |
| | | gtgcctggccacaggcttcttccctgaccacgtggagctgagctggtgggtgaatgggaaggaggtgc |
| | | acagtggggtcagcacggacccgcagcccctcaaggagcagcccgccctcaatgactccagatactg |
| | | cctgagcagccgcctgagggtctcggccaccttctggcagaacccccgcaaccacttccgctgtcaagt |
| | | ccagttctacgggctctcggagaatgacgagtggacccaggatagggccaaacccgtcacccagatcg |
| | | tcagcgccgaggcctggggtagagcagactgtggctttacctcggtgtcctaccagcaaggggtcctgt |
| | | ctgccaccatcctctatgagatcctgctagggaaggccaccctgtatgctgtgctggtcagcgcccttgtg |
| | | ttgatggccatggtcaagagaaaggatttcTAA |
| 667 | RAS | atgaagcccaccctcatctcagtgcttgtgataatatttatactcagaggaacaagagcccagagagtgac |
| | | tcagcccgagaagctcctctctgtctttaaaggggccccagtggagctgaagtgcaactattcctattctg |
| | | ggagtcctgaactcttctggtatgtccagtactccagacaacgcctccagttactcttgagacacatctcta |
| | | gagagagcatcaaaggcttcactgctgaccttaacaaaggcgagacatctttccacctgaagaaaccatt |
| | | tgctcaagaggaagactcagccatgtattactgtgctctaagtactggaggcttcaaaactatctttggagc |
| | | aggaacaagactatttgttaaagcaaatatccagaaccctgaccctgccgtgtaccagctgagagactct |
| | | aaatccagtgacaagtctgtctgcctattcaccgattttgattctcaaacaaatgtgtcacaaagtaaggatt |
| | | ctgatgtgtatatcacagacaaaactgtgctagacatgaggtctatggacttcaagagcaacagtgctgtg |
| | | gcctggagcaacaaatctgactttgcatgtgcaaacgccttcaacaacagcattattccagaagacacctt |
| | | cttccccagcccagaaagttcctgtgatgtcaagctggtcgagaaaagctttgaaacagatacgaaccta |
| | | aactttcaaaacctgtcagtgattgggttccgaatcctcctcctgaaagtggccgggtttaatctgctcatga |
| | | cgctgcggctgtggtccagcTAA |
| 668 | RAS | atgctgagtcttctgctccttctcctgggactaggctctgtgttcagtgctgtcatctctcaaaagccaagca |
| | | gggatatctgtcaacgtggaacctccctgacgatccagtgtcaagtcgatagccaagtcaccatgatgttc |
| | | tggtaccgtcagcaacctggacagagcctgacactgatcgcaactgcaaatcagggctctgaggccac |
| | | atatgagagtggatttgtcattgacaagtttcccatcagccgcccaaacctaacattctcaactctgactgtg |
| | | agcaacatgagccctgaagacagcagcatatatctctgcagcgttttgggtcccgggacagggggacg |
| | | aggagctaactatggctacaccttcggttcggggaccaggttaaccgttgtagaggacctgaacaaggt |
| | | gttcccacccgaggtcgctgtgtttgagccatcagaagcagagatctcccacacccaaaaggccacact |
| | | ggtgtgcctggccacaggcttcttccctgaccacgtggagctgagctggtgggtgaatgggaaggaggt |
| | | gcacagtggggtcagcacggacccgcagcccctcaaggagcagcccgccctcaatgactccagatac |
| | | tgcctgagcagccgcctgagggtctcggccaccttctggcagaacccccgcaaccacttccgctgtcaa |
| | | gtccagttctacgggctctcggagaatgacgagtggacccaggatagggccaaacccgtcacccagat |
| | | cgtcagcgccgaggcctggggtagagcagactgtggctttacctcggtgtcctaccagcaaggggtcct |
| | | gtctgccaccatcctctatgagatcctgctagggaaggccaccctgtatgctgtgctggtcagcgcccttg |
| | | tgttgatggccatggtcaagagaaaggatttcTAA |
| 669 | RAS | atggaaactctcctgggagtgtctttggtgattctatggcttcaactggctagggtgaacagtcaacaggg |
| | | agaagaggatcctcaggccttgagcatccaggagggtgaaaatgccaccatgaactgcagttacaaaa |
| | | ctagtataaacaatttacagtggtatagacaaaattcaggtagaggccttgtccacctaattttaatacgttc |
| | | aaatgaaagagagaaacacagtggaagattaagagtcacgcttgacacttccaagaaaagcagttccttgt |
| | | tgatcacggcttcccgggcagcagacactgcttcttacttctgtgctaccactatagatggccagaagctg |
| | | ctctttgcaaggggaaccatgttaaaggtggatcttaatatccagaaccctgaccctgccgtgtaccagct |
| | | gagagactctaaatccagtgacaagtctgtctgcctattcaccgattttgattctcaaacaaatgtgtcacaa |
| | | agtaaggattctgatgtgtatatcacagacaaaactgtgctagacatgaggtctatggacttcaagagcaa |
| | | cagtgctgtggcctggagcaacaaatctgactttgcatgtgcaaacgccttcaacaacagcattattccag |
| | | aagacaccttcttccccagcccagaaagttcctgtgatgtcaagctggtcgagaaaagctttgaaacagat |
| | | acgaacctaaactttcaaaacctgtcagtgattgggttccgaatcctcctcctgaaagtggccgggtttaat |
| | | ctgctcatgacgctgcggctgtggtccagcTAA |
| 670 | RAS | atgctgctgcttctgctgcttctggggccagcaggctccgggcttggtgctgtcgtctctcaacatccgag |
| | | cagggttatctgtaagagtggaacctctgtgaagatcgagtgccgttccctggactttcaggccacaacta |
| | | tgttttggtatcgtcagttcccgaaaaagagtctcatgctgatggcaacttccaatgagggctccaaggcc |
| | | acatacgagcaaggcgtcgagaaggacaaqtttctcatcaaccatqcaaqcctqaccttqtccactctqa |
| | | caqtqaccaqtqcccatcctgaagacagcagcttctacatctgcagtgcttctgacagggggtccgggg |
| | | agctgttttttggagaaggctctaggctgaccgtactggaggacctgaacaaggtgttcccacccgaggt |
| | | cgctgtgtttgagccatcagaagcagagatctcccacacccaaaaggccacactggtgtgcctggccac |
| | | aggcttcttccctgaccacgtggagctgagctggtgggtgaatgggaaggaggtgcacagtggggtca |
| | | gcacggacccgcagcccctcaaggagcagcccgccctcaatgactccagatactgcctgagcagccg |
| | | cctgagggtctcggccaccttctggcagaacccccgcaaccacttccgctgtcaagtccagttctacggg |
| | | ctctcggagaatgacgagtggacccaggatagggccaaacccgtcacccagatcgtcagcgccgagg |
| | | cctggggtagagcagactgtggctttacctcggtgtcctaccagcaaggggtcctgtctgccaccatcct |
| | | ctatgagatcctgctagggaaggccaccctgtatgctgtgctggtcagcgcccttgtgttgatggccatgg |
| | | tcaagagaaaggatttcTAA |
| 671 | RAS | atggaaactctcctgggagtgtctttggtgattctatggcttcaactggctagggtgaacagtcaacaggg |
| | | agaagaggatcctcaggccttgagcatccaggagggtgaaaatgccaccatgaactgcagttacaaaa |
| | | ctagtataaacaatttacagtggtatagacaaaattcaggtagaggccttgtccacctaattttaatacgttc |
| | | aaatgaaagagagaaacacagtggaagattaagagtcacgcttgacacttccaagaaaagcagttccttgt |
| | | tgatcacggcttcccgggcagcagacactgcttcttacttctgtgctacggacgcatacaatgccagactc |
| | | atgtttggagatggaactcagctggtggtgaagcccaatatccagaaccctgaccctgccgtgtaccagc |
| | | tgagagactctaaatccagtgacaagtctgtctgcctattcaccgattttgattctcaaacaaatgtgtcaca |
| | | aagtaaggattctgatgtgtatatcacagacaaaactgtgctagacatgaggtctatggacttcaagagca |
| | | acagtgctgtggcctggagcaacaaatctgactttgcatgtgcaaacgccttcaacaacagcattattcca |
| | | gaagacaccttcttccccagcccagaaagttcctgtgatgtcaagctggtcgagaaaagctttgaaacag |
| | | atacgaacctaaactttcaaaacctgtcagtgattgggttccgaatcctcctcctgaaagtggccgggttta |
| | | atctgctcatgacgctgcggctgtggtccagcTAA |
| 672 | RAS | atgctgagtcttctgctccttctcctgggactaggctctgtgttcagtgctgtcatctctcaaaagccaagca |
| | | gggatatctgtcaacgtggaacctccctgacgatccagtgtcaagtcgatagccaagtcaccatgatgttc |
| | | tggtaccgtcagcaacctggacagagcctgacactgatcgcaactgcaaatcagggctctgaggccac |
| | | atatgagagtggatttgtcattgacaagtttcccatcagccgcccaaacctaacattctcaactctgactgtg |
| | | agcaacatgagccctgaagacagcagcatatatctctgcagcgttgtggcaagcgggagtgtagatacg |
| | | cagtattttggcccaggcacccggctgacagtgctcgaggacctgaacaaggtgttcccacccgaggtc |
| | | gctgtgtttgagccatcagaagcagagatctcccacacccaaaaggccacactggtgtgcctggccaca |
| | | ggcttcttccctgaccacgtggagctgagctggtgggtgaatgggaaggaggtgcacagtggggtcag |
| | | cacggacccgcagcccctcaaggagcagcccgccctcaatgactccagatactgcctgagcagccgc |
| | | ctgagggtctcggccaccttctggcagaacccccgcaaccacttccgctgtcaagtccagttctacgggc |
| | | tctcggagaatgacgagtggacccaggatagggccaaacccgtcacccagatcgtcagcgccgaggc |
| | | ctggggtagagcagactgtggctttacctcggtgtcctaccagcaaggggtcctgtctgccaccatcctct |
| | | atgagatcctgctagggaaggccaccctgtatgctgtgctggtcagcgcccttgtgttgatggccatggtc |
| | | aagagaaaggatttcTAA |
| 673 | RAS | atggtcctgaaattctccgtgtccattctttggattcagttggcatgggtgagcacccagctgctggagcag |
| | | agccctcagtttctaagcatccaagagggagaaaatctcactgtgtactgcaactcctcaagtgttttttcca |
| | | gcttacaatggtacagacaggagcctggggaaggtcctgtcctcctggtgacagtagttacgggtggag |
| | | aagtgaagaagctgaagagactaacctttcagtttggtgatgcaagaaaggacagttctctccacatcact |
| | | gcagcccagcctggtgatacaggcctctacctctgtgcaggtcctgcgggagcccagaagctggtattt |
| | | ggccaaggaaccaggctgactatcaacccaaatatccagaaccctgaccctgccgtgtaccagctgag |
| | | agactctaaatccagtgacaagtctgtctgcctattcaccgattttgattctcaaacaaatgtgtcacaaagt |
| | | aaggattctgatgtgtatatcacagacaaaactgtgctagacatgaggtctatggacttcaagagcaacag |
| | | tgctgtggcctggagcaacaaatctgactttgcatgtgcaaacgccttcaacaacagcattattccagaag |
| | | acaccttcttccccagcccagaaagttcctgtgatgtcaagctggtcgagaaaagctttgaaacagatacg |
| | | aacctaaactttcaaaacctgtcagtgattgggttccgaatcctcctcctgaaagtggccgggtttaatctgc |
| | | tcatgacgctgcggctgtggtccagcTAA |
| 674 | RAS | Atgagcatcagcctcctgtgctgtgcagcctttcctctcctgtgggcaggtccagtgaatgctggtgtcac |
| | | tcagaccccaaaattccgcatcctgaagataggacagagcatgacactgcagtgtacccaggatatgaa |
| | | ccataactacatgtactggtatcgacaagacccaggcatggggctgaagctgatttattattcagttggtgc |
| | | tggtatcactgataaaggagaagtcccgaatggctacaacgtctccagatcaaccacagaggatttcccg |
| | | ctcaggctggagttggctgctccctcccagacatctgtgtacttctgtgccagcagccccgtaccctactct |
| | | ggaaacaccatatattttggagagggaagttggctcactgttgtagaggacctgaacaaggtgttcccac |
| | | ccgaggtcgctgtgtttgagccatcagaagcagagatctcccacacccaaaaggccacactggtgtgcc |
| | | tggccacaggcttcttccctgaccacgtggagctgagctggtgggtgaatgggaaggaggtgcacagt |
| | | ggggtcagcacggacccgcagcccctcaaggagcagcccgccctcaatgactccagatactgcctga |
| | | gcagccgcctgagggtctcggccaccttctggcagaacccccgcaaccacttccgctgtcaagtccagt |
| | | tctacgggctctcggagaatgacgagtggacccaggatagggccaaacccgtcacccagatcgtcagc |
| | | gccgaggcctggggtagagcagactgtggctttacctcggtgtcctaccagcaaggggtcctgtctgcc |
| | | accatcctctatgagatcctgctagggaaggccaccctgtatgctgtgctggtcagcgcccttgtgttgatg |
| | | gccatggtcaagagaaaggatttcTAA |
| 675 | RAS | atgctcctgctgctcgtcccagtgctcgaggtgatttttaccctgggaggaaccagagcccagtcggtga |
| | | cccagcttggcagccacgtctctgtctctgaaggagccctggttctgctgaggtgcaactactcatcgtct |
| | | gttccaccatatctcttctggtatgtgcaataccccaaccaaggactccagcttctcctgaagtacacatca |
| | | gcggccaccctggttaaaggcatcaacggttttgaggctgaatttaagaagagtgaaacctccttccacct |
| | | gacgaaaccctcagcccatatgagcgacgcggctgagtacttctgtgctgtgagtaagagtgacaagat |
| | | catctttggaaaagggacacgacttcatattctccccaatatccagaaccctgaccctgccgtgtaccagc |
| | | tgagagactctaaatccagtgacaagtctgtctgcctattcaccgattttgattctcaaacaaatgtgtcaca |
| | | aagtaaggattctgatgtgtatatcacagacaaaactgtgctagacatgaggtctatggacttcaagagca |
| | | acagtgctgtggcctggagcaacaaatctgactttgcatgtgcaaacgccttcaacaacagcattattcca |
| | | gaagacaccttcttccccagcccagaaagttcctgtgatgtcaagctggtcgagaaaagctttgaaacag |
| | | atacgaacctaaactttcaaaacctgtcagtgattgggttccgaatcctcctcctgaaagtggccgggttta |
| | | atctgctcatgacgctgcggctgtggtccagcTAA |
| 676 | RAS | atggcctccctgctcttcttctgtggggccttttatctcctgggaacagggtccatggatgctgatgttaccc |
| | | agaccccaaggaataggatcacaaagacaggaaagaggattatgctggaatgttctcagactaagggtc |
| | | atgatagaatgtactggtatcgacaagacccaggactgggcctacggttgatctattactcctttgatgtca |
| | | aagatataaacaaaggagagatctctgatggatacagtgtctctcgacaggcacaggctaaattctccctg |
| | | tccctagagtctgccatccccaaccagacagctctttacttctgtgccaccagtgagggggggagtacgg |
| | | gcactgaagctttctttggacaaggcaccagactcacagttgtagaggacctgaacaaggtgttcccacc |
| | | cgaggtcgctgtgtttgagccatcagaagcagagatctcccacacccaaaaggccacactggtgtgcct |
| | | ggccacaggcttcttccctgaccacgtggagctgagctggtgggtgaatgggaaggaggtgcacagtg |
| | | gggtcagcacggacccgcagcccctcaaggagcagcccgccctcaatgactccagatactgcctgag |
| | | cagccgcctgagggtctcggccaccttctggcagaacccccgcaaccacttccgctgtcaagtccagtt |
| | | ctacgggctctcggagaatgacgagtggacccaggatagggccaaacccgtcacccagatcgtcagc |
| | | gccgaggcctggggtagagcagactgtggctttacctcggtgtcctaccagcaaggggtcctgtctgcc |
| | | accatcctctatgagatcctgctagggaaggccaccctgtatgctgtgctggtcagcgcccttgtgttgatg |
| | | gccatggtcaagagaaaggatttcTAA |
| 677 | RAS | atgaactattctccaggcttagtatctctgatactcttactgcttggaagaacccgtggaaattcagtgaccc |
| | | agatggaagggccagtgactctctcagaagaggccttcctgactataaactgcacgtacacagccacag |
| | | gatacccttcccttttctggtatgtccaatatcctggagaaggtctacagctcctcctgaaagccacgaagg |
| | | ctgatgacaagggaagcaacaaaggttttgaagccacataccgtaaagaaaccacttctttccacttgga |
| | | gaaaggctcagttcaagtgtcagactcagcggtgtacttctgtgctctgaataggaacaccggtaaccagt |
| | | tctattttgggacagggacaagtttgacggtcattccaaatatccagaaccctgaccctgccgtgtaccag |
| | | ctgagagactctaaatccagtgacaagtctgtctgcctattcaccgattttgattctcaaacaaatgtgtcac |
| | | aaagtaaggattctgatgtgtatatcacagacaaaactgtgctagacatgaggtctatggacttcaagagc |
| | | aacagtgctgtggcctggagcaacaaatctgactttgcatgtgcaaacgccttcaacaacagcattattcc |
| | | agaagacaccttcttccccagcccagaaagttcctgtgatgtcaagctggtcgagaaaagctttgaaaca |
| | | gatacgaacctaaactttcaaaacctgtcagtgattgggttccgaatcctcctcctgaaagtggccgggttt |
| | | aatctgctcatgacgctgcggctgtggtccagcTAA |
| 678 | RAS | atgagcaaccaggtgctctgctgtgtggtcctttgtctcctgggagcaaacaccgtggatggtggaatcac |
| | | tcagtccccaaagtacctgttcagaaaggaaggacagaatgtgaccctgagttgtgaacagaatttgaac |
| | | cacgatgccatgtactggtaccgacaggacccagggcaagggctgagattgatctactactcacagata |
| | | gtaaatgactttcagaaaggagatatagctgaagggtacagcgtctctcgggagaagaaggaatcctttc |
| | | ctctcactgtgacatcggcccaaaagaacccgacagctttctatctctgtgccagtagtatcccacagggc |
| | | aatggctacaccttcggttcggggaccaggttaaccgttgtagaggacctgaacaaggtgttcccacccg |
| | | aggtcgctgtgtttgagccatcagaagcagagatctcccacacccaaaaggccacactggtgtgcctgg |
| | | ccacaggcttcttccctgaccacgtggagctgagctggtgggtgaatgggaaggaggtgcacagtggg |
| | | gtcagcacggacccgcagcccctcaaggagcagcccgccctcaatgactccagatactgcctgagca |
| | | gccgcctgagggtctcggccaccttctggcagaacccccgcaaccacttccgctgtcaagtccagttcta |
| | | cgggctctcggagaatgacgagtggacccaggatagggccaaacccgtcacccagatcgtcagcgcc |
| | | gaggcctggggtagagcagactgtggctttacctcggtgtcctaccagcaaggggtcctgtctgccacc |
| | | atcctctatqaqatcctqctaggaaggccaccctgtatgctgtgctggtcagcgcccttgtgttgatggcca |
| | | tggtcaagagaaaggatttcTAA |
| 679 | RAS | Tgtgctgtgagtgtaggtcctggcaacacaggcaaactaatcttt |
| 680 | RAS | Tgtgccagcagtttgggggttctaggcctgcggtacttc |
| 681 | RAS | Tgtgcagagaatagtggaggtagcaactataaactgacattt |
| 682 | RAS | Tgtgccagcagctgggaaagggcggggaaagctttcttt |
| 683 | RAS | Tgtgcaatgagcgtctttatttatagcacattcatcttt |
| 684 | RAS | Tgtgccagctctgggaggcaagagacccagtacttc |
| 685 | RAS | Tgtgctacggacgcgtacacaaggcaactgaccttt |
| 686 | RAS | Tgtgccagtgggggaccgggggctaacaggccccagcatttt |
| 687 | RAS | Tgtgcttataggagcctttgggggtccgggtatgcactcaacttc |
| 688 | RAS | Tgcagcgttgaaaagggcgcgcaagagacccagtacttc |
| 689 | RAS | Tgtgcagcactctttggaaatgagaaattaaccttt |
| 690 | RAS | Tgtgccaccctacagggttggtcttataatgagcagttcttc |
| 691 | RAS | Tgtgctctaagtactggaggcttcaaaactatcttt |
| 692 | RAS | Tgcagcgttttgggtcccgggacagggggacgaggagctaactatggctacaccttc |
| 693 | RAS | Tgtgctaccactatagatggccagaagctgctcttt |
| 694 | RAS | Tgcagtgcttctgacagggggtccggggagctgtttttt |
| 695 | RAS | Tgtgctacggacgcatacaatgccagactcatgttt |
| 696 | RAS | Tgcagcgttgtggcaagcgggagtgtagatacgcagtatttt |
| 697 | RAS | Tgtgcaggtcctgcgggagcccagaagctggtattt |
| 698 | RAS | Tgtgccagcagccccgtaccctactctggaaacaccatatatttt |
| 699 | RAS | Tgtgctgtgagtaagagtgacaagatcatcttt |
| 700 | RAS | Tgtgccaccagtgagggggggagtacgggcactgaagctttcttt |
| 701 | RAS | Tgtgctctgaataggaacaccggtaaccagttctatttt |
| 702 | RAS | Tgtgccagtagtatcccacagggcaatggctacaccttc |
| 703 | RAS/P53 | DIQNPDPAVYQLRDSKSSDKSVCLFTDFDSQTNVSQSKDSDVYIT |
| | | DKTVLDMRSMDFKSNSAVAWSNKSDFACANAFNNSIIPEDTFFPS |
| | | PESSCDVKLVEKSFETDTNLNFQNLSVIGFRILLLKVAGFNLLMTL |
| | | RLWSS |
| 704 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 705 | RAS/P53 | IQNPDPAVYQLRDSKSSDKSVCLFTDFDSQTNVSQSKDSDVYITD |
| | | KCVLDMRSMDFKSNSAVAWSNKSDFACANAFNNSIIPEDTFFPSS |
| | | DVPCDVKLVEKSFETDTNLNFQNLSVIGFRILLLKVAGFNLLMTLR |
| | | LWSS |
| 706 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 707 | RAS/P53 | IQNPDPAVYQLRDSKSSDKSVCLFTDFDSQTNVSQSKDSDVYITD |
| | | KCVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 708 | RAS/P53 | PDPAVYQLRDSKSSDKSVCLFTDFDSQTNVSQSKDSDVYITDKCV |
| | | LDMRSMDFKSNSAVAWSNKSDFACANAFNNSIIPEDTFFPSPESSC |
| | | DVKLVEKSFETDTNLNFQNLSVIGFRILLLKVAGFNLLMTLRLWSS |
| 709 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSS |
| | | DVPCDVKLVEKSFETDTNLNFQNLSVIGFRILLLKVAGFNLLMTLR |
| | | LWSS |
| 710 | RAS/P53 | IQNPDPAVYQLRDSKSSDKSVCLFTDFDSQTNVSQSKDSDVYITD |
| | | KTVLDMRSMDFKSNRAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 711 | RAS/P53 | IQNPDPAVYQLRDSKSSDKSVCLFTDFDSQTNVSQSKDSDVYITD |
| | | KTVLDMRSMDFKSNRAVAWSNKSDFACANAFNNSIIPEDTFFPSS |
| | | DVPCDVKLVEKSFETDTNLNFQNLSVIGFRILLLKVAGFNLLMTLR |
| | | LWSS |
| 712 | RAS/P53 | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCGTQSFGLLDPKLCYLLDGILFIYGVILTALFLRVKFSRSADAP |
| | | AYQQGQNQLYNELNLGRREEYDVLDKRRGRDPEMGGKPQRRKN |
| | | PQEGLYNELQKDKMAEAYSEIGMKGERRRGKGHDGLYQGLSTA |
| | | TKDTYDALHMQALPPR |
| 713 | RAS/P53 | EDLNKVFPPEVAVFEPSEAEISHTQKATLVCLATGFFPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSVSYQQGVLSATILYEILLGKATLYAVLVSALVLMAMVKRKDF |
| 714 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 715 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 716 | RAS/P53 | EDLKNVFPPEVAVFEPSEAEISHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 717 | RAS/P53 | EDLKNVFPPEVAVFEPSKAEIAHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDSR |
| | | G |
| 718 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 719 | RAS/P53 | EDLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDSR |
| | | G |
| 720 | RAS/P53 | EDLKNVFPPEVAVFEPSEAEISHTQKATLVCLATGFYPDHVELSW |
| | | WVNGKEVHSGVSTDPQPLKEQPALNDSRYCLSSGLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | G |
| 721 | RAS/P53 | EDLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGS |
| | | QSFGLLDPKLCYLLDGILFIYGVILTALFLRVKFSRSADAPAYQQG |
| | | QNQLYNELNLGRREEYDVLDKRRGRDPEMGGKPQRRKNPQEGL |
| | | YNELQKDKMAEAYSEIGMKGERRRGKGHDGLYQGLSTATKDTY |
| | | DALHMQALPPR |
| 722 | RAS/P53 | GCPALPTGVGGTPFPSLAPPIMLLVDGKQQMVVVCLVLDVAPPGL |
| | | DSPIWFSAGNGSALDAFTYGPSPATDGTWTNLAHLSLPSEELASW |
| | | EPLVCHTGPGAEGHSRSTQPMHLSGEASTARTCPQEPLRGTPGGA |
| | | LWLGVLRLLLFKLLLFDLLLTCSCLCDPAGPLPSPATTTRLRALG |
| | | SHRLHPATETGGREATSSPRPQPRDRRWGDTPPGRKPGSPVWGEG |
| | | SYLSSYPTCPAQAWC |
| | | SRSRLRAPSSSLGAFFAGDLPPPLQAGAA |
| 723 | RAS/P53 | TPFPSLAPPIMLLVDGKQQMVVVCLVLDVAPPGLDSPIWFSAGNG |
| | | SALDAFTYGPSPATDGTWTNLAHLSLPSEELASWEPLVCHTGPGA |
| | | EGHSRSTQPMHLSGEASTARTCPQEPLRGTPGGALWLGVLRLLLF |
| | | KLLLFDLLLTCSCLCDPAGPLPSPATTTRLRALGSHRLHPATETG |
| | | GREATSSPRPQPRDRRWGDTPPGRKPGSPVWGEGSYLSSYPTCPA |
| | | QAWCSRSALRAPSSSLGAFFAGDLPPPLQAGAA |
| 724 | RAS/P53 | TPFPSLAPPIMLLVDGKQQMVVVCLVLDVAPPGLDSPIWFSAGNG |
| | | SALDAFTYGPSPATDGTWTNLAHLSLPSEELASWEPLVCHTGPGA |
| | | EGHSRSTQPMHLSGEASTARTCPQEPLRGTPGGALWLGVLRLLLF |
| | | KLLLFDLLLTCSCLCDPAGPLPSPATTTRLRALGSHRLHPATETGG |
| | | REATSSPRPQPRDRRWGDTPPGRKPGSPV |
| 725 | RAS/P53 | DKQLDADVSPKPTIFLPSIAETKLQKAGTYLCLLEKFFPDIIKIHWQ |
| | | EKKSNTILGSQEGNTMKTNDTYMKFSWLTVPEESLDKEHRCIVRH |
| | | ENNKNGIDQEIIFPPIKTDVTTVDPKYNYSKDANDVITMDPKDNW |
| | | SKDANDTLLLQLTNTSAYYMYLLLLLKSVVYFAIITCCLLGRTAFC |
| | | CNGEKS |
| 726 | RAS/P53 | DKQLDADVSPKPTIFLPSIAETKLQKAGTYLCLLEKFFPDVIKIHW |
| | | QEKKSNTILGSQEGNTMKTNDTYMKFSWLTVPEKSLDKEHRCIV |
| | | RHENNKNGVDQEIIFPPIKTDVITMDPKDNCSKDANDTLLLQLTNT |
| | | SAYYMYLLLLLKSVVYFAIITCCLLRRTAFCCNGEKS |
| 727 | RAS/P53 | SQPHTKPSVFVMKNGTNVACLVKEFYPKDIRINLVSSKKITEFDPA |
| | | IVISPSGKYNAVKLGKYEDSNSVTCSVQHDNKTVHSTDFEVKTDS |
| | | TDHVKPKETENTKQPSKSCHKPKAIVHTEKVNMMSLTVLGLRML |
| | | FAKTVAVNFLLTAKLFFL |
| 728 | RAS/P53 | SQPHTKPSVFVMKNGTNVACLVKEFYPKDIRINLVSSKKITEFDPA |
| | | IVISPSGKYNAVKLGKYEDSNSVTCSVQHDNKTVHSTDFEVKTDS |
| | | TDHVKPKETENTKQPSKSCHKPKAIVHTEKVNMMSLTVLGLRML |
| | | FAKTVAVNFLLTAKLFFL |
| 729 | RAS/P53 | KNQVEQSPQSLIILEGKNCTLQCNYTVSPFSNLRWYKQDTGRGPV |
| | | SLTIMTFSENTKSNGRYTATLDADTKQSSLHITASQLSDSASYICV |
| | | VSDRGSTLGRLYFGRGTQLTVWPD |
| 730 | RAS/P53 | DIYQTPRYLVIGTGKKITLECSQTMGHDKMYWYQQDPGMELHLI |
| | | HYSYGVNSTEKGDLSSESTVSRIRTEHFPLTLESARPSHTSQYLCAS |
| | | SENIGTAYEQYFGPGTRLTV |
| 731 | RAS/P53 | GVTQSPTHLIKTRGQQATLRCSPISGHTSVYWYQQALGLGLQFLL |
| | | WYDEGEERNRGNFPPRFSGRQFPNYSSELNVNALELEDSALYLCA |
| | | SSLGGPRGLAGLRGDEQFFGPGTRLTVLE |
| 732 | RAS/P53 | GVTQSPTHLIKTRGQQATLRCSPISGHTSVYWYQQALGLGLQFLL |
| | | WYDEGEERNRGNFPPRFSGRQFPNYSSELNVNALELEDSALYLCA |
| | | SSLGGDELGADGNEQFFGPGTRLTVLE |
| 733 | RAS/P53 | AQTVTQSQPEMSVQEAETVTLSCTYDTSESDYYLFWYKQPPSRQ |
| | | MILVIRQEAYKQQNATENRFSVNFQKAAKSFSLKISDSQLGDAAM |
| | | YFCAYRSAVNARLMFGDGTQLVVKPN |
| 734 | RAS/P53 | EADIYQTPRYLVIGTGKKITLECSQTMGHDKMYWYQQDPGMELH |
| | | LIHYSYGVNSTEKGDLSSESTVSRIRTEHFPLTLESARPSHTSQYLC |
| | | ASSEARGLAEFTDTQYFGPGTRLTVLE |
| 735 | RAS/P53 | QEVTQIPAALSVPEGENLVLNCSFTDSAIYNLQWFRQDPGKGLTSL |
| | | LLIQSSQREQTSGRLNASLDKSSGRSTLYIAASQPGDSATYLCAVR |
| | | PTSGGSYIPTFGRGTSLIVHPY |
| 736 | RAS/P53 | NAGVTQTPKFQVLKTGQSMTLQCAQDMNHEYMSWYRQDPGMG |
| | | LRLIHYSVAIQTTDQGEVPNGYNVSRSTIEDFPLRLLSAAPSQTSVY |
| | | FCASSYLGNTGELFFGEGSRLTVLE |
| 737 | RAS/P53 | ILNVEQSPQSLHVQEGDSTNFTCSFPSSNFYALHWYRWETAKSPE |
| | | ALFVMTLNGDEKKKGRISATLNTKEGYSYLYIKGSQPEDSATYLC |
| | | ARNTGNQFYFGTGTSLTVIP |
| 738 | RAS/P53 | GVTQTPKFQVLKTGQSMTLQCAQDMNHEYMSWYRQDPGMGLR |
| | | LIHYSVGAGITDQGEVPNGYNVSRSTTEDFPLRLLSAAPSQTSVYF |
| | | CASSFQTGASYGYTFGSGTRLTVLE |
| 739 | RAS/P53 | AIELVPEHQTVPVSIGVPATLRCSMKGEAIGNYYINWYRKTQGNT |
| | | MTFIYREKDIYGPGFKDNFQGDIDIAKNLAVLKILAPSERDEGSYY |
| | | CACDTLGMGGEYTDKLIFGKGTRVTVEPR |
| 740 | RAS/P53 | AGHLEQPQISSTKTLSKTARLECVVSGITISATSVYWYRERPGEVIQ |
| | | FLVSISYDGTVRKESGIPSGKFEVDRIPETSTSTLTIHNVEKQDIATY |
| | | YCALWEAQQELGKKIKVFGPGTKLIITDKQL |
| 741 | P53 | QDVNTA |
| 742 | P53 | SAS |
| 743 | P53 | SAY |
| 744 | P53 | QQYSRYSPVTF |
| 745 | P53 | QQQSSTPVTF |
| 746 | P53 | QQSSYYPNTF |
| 747 | P53 | QQQWSSPDTF |
| 748 | P53 | QQSNAYPITF |
| 749 | P53 | GFNVYASGM |
| 750 | P53 | GFNVYQSDM |
| 751 | P53 | GFNLYQRDM |
| 752 | P53 | GFNLSYYDM |
| 753 | P53 | GFNLNSYYM |
| 754 | P53 | KIYPDSDYTY |
| 755 | P53 | TIWPYSGYTY |
| 756 | P53 | GLLYGSDHTE |
| 757 | P53 | LIYYGSGYTY |
| 758 | P53 | MIIPGYGYTN |
| 759 | P53 | SRDSSFYYVYAMDY |
| 760 | P53 | SRDGMYAFDY |
| 761 | P53 | SRATYEEAFDY |
| 762 | P53 | SRGYVSGMDY |
| 763 | P53 | SRSYYMYMDY |
| 764 | RAS | QDVNTA |
| 765 | RAS | SAS |
| 766 | RAS | QQVIYYPFTF |
| 767 | RAS | QQYDYYPFTF |
| 768 | RAS | QQSIYYPFTF |
| 769 | RAS | QQSSYSPWTF |
| 770 | RAS | QQSFSTPITF |
| 771 | RAS | QQGEYSPLTF |
| 772 | RAS | QQTYYTPVTF |
| 773 | RAS | GFNLYSYAI |
| 774 | RAS | GFNISYEAM |
| 775 | RAS | GFNLYTSQM |
| 776 | RAS | GFNVFGYAI |
| 777 | RAS | GFNISPWDM |
| 778 | RAS | GFNISEYLM |
| 779 | RAS | GFNVFESAM |
| 780 | RAS | GFNISHYVM |
| 781 | RAS | LLYPDYGVTS |
| 782 | RAS | LIYPNHGITS |
| 783 | RAS | LVYPGYYVTS |
| 784 | RAS | EVYPGYDVTS |
| 785 | RAS | QLYPSSGYTN |
| 786 | RAS | LLPPGLSYTN |
| 787 | RAS | WVYGSYDYTY |
| 788 | RAS | DFYPHSDSTY |
| 789 | RAS | SRYRSYEYSVSSYSYSAMDY |
| 790 | RAS | SRYSSSAMDY |
| 791 | RAS | SRGAYYYSSAMDY |
| 792 | RAS | SRYSWAGAFDY |
| 793 | RAS | SRSVYWSLDY |
| 794 | RAS | SRYGYYAFDY |
| 795 | RAS | SRSFAYFQAMDY |
| 796 | RAS | SRYQSYSFDY |
| 797 | RAS | QQASRQPYTF |
| 798 | RAS | QQAVSYPWTF |
| 799 | RAS | QQTSSYPITF |
| 800 | RAS | QQSWYSPSTF |
| 801 | RAS | QQSYYAPITF |
| 802 | RAS | QQSYYSPWTF |
| 803 | RAS | QQAYYPPWTF |
| 804 | RAS | QQSYSSGPVTF |
| 805 | RAS | QQTYYYPFTF |
| 806 | RAS | QQSYYPYYPWTF |
| 807 | RAS | QQYDRPITF |
| 808 | RAS | GFNFSESGM |
| 809 | RAS | GFNISSSGI |
| 810 | RAS | GFNIYWYGM |
| 811 | RAS | GFNISASGM |
| 812 | RAS | GFNFSYYGM |
| 813 | RAS | GENISYSNI |
| 814 | RAS | GFNVSRWAM |
| 815 | RAS | GFNFSYGGI |
| 816 | RAS | GFNLYAWGM |
| 817 | RAS | GFNVSHSAM |
| 818 | RAS | GFNIYYEAM |
| 819 | RAS | HFSGDSGYTY |
| 820 | RAS | MVYGGSGYTN |
| 821 | RAS | QVYPWSGFTY |
| 822 | RAS | WIWGGSSYTY |
| 823 | RAS | WIYPFSGYTN |
| 824 | RAS | MIYGTRGGTY |
| 825 | RAS | RVYPSGYLTY |
| 826 | RAS | MIYPLTGYTN |
| 827 | RAS | LVYGGWGSTS |
| 828 | RAS | TVHPDWGNTY |
| 829 | RAS | QIYPWNDYTY |
| 830 | RAS | SRYMYYSGYFDY |
| 831 | RAS | SRWAHYSAYMDY |
| 832 | RAS | SRDYYSYSLDY |
| 833 | RAS | SRGQYLSYMDY |
| 834 | RAS | SREYYSRAFDY |
| 835 | RAS | SRYYSYAMDY |
| 836 | RAS | SRNMQSYMDY |
| 837 | RAS | SRDYYYSVDV |
| 838 | RAS | SRAGSSKMSAGAFDY |
| 839 | RAS | SRWQQYYYSFDY |
| 840 | RAS | SRNYYAATMDY |
| 841 | RAS | QQSYTSPLTF |
| 842 | RAS | QQYWYYYPITF |
| 843 | RAS | QQSYYAPITF |
| 844 | RAS | QQYYLYQPITF |
| 845 | RAS | QQYSNYPLTF |
| 846 | RAS | QQYASDPITF |
| 847 | RAS | QQYSYDPITF |
| 848 | RAS | QQYIYDPVTF |
| 849 | RAS | QQLMYDPITF |
| 850 | RAS | GFNIYYGVM |
| 851 | RAS | GFNIYSYDM |
| 852 | RAS | GFNVQWSHM |
| 853 | RAS | GFNIGMYTM |
| 854 | RAS | GFNVFYGSM |
| 855 | RAS | GFNLDYGWM |
| 856 | RAS | GFNFSYSAM |
| 857 | RAS | GFNVDWAWM |
| 858 | RAS | GFNFGTYWM |
| 859 | RAS | MIYPDSSWTY |
| 860 | RAS | ISPGGSYTY |
| 861 | RAS | RLSPPSGYTN |
| 862 | RAS | LVYPDSGYTN |
| 863 | RAS | FIGPDSTYTY |
| 864 | RAS | WVVPGSDYTD |
| 865 | RAS | DVVPDGDWTY |
| 866 | RAS | WVVGGSDYTY |
| 867 | RAS | WFLPDYDYTL |
| 868 | RAS | SRDQDFHYMNYYLSYALDY |
| 869 | RAS | SRSAFTGYFDV |
| 870 | RAS | SRLILSKGGYGWAMDY |
| 871 | RAS | SRYTWQSMDY |
| 872 | RAS | SRDLGSAYAMDY |
| 873 | RAS | SRFHYTAFDV |
| 874 | RAS | SRGWYALDY |
| 875 | RAS | SRSYYYAFDY |
| 876 | RAS | SRHGEYAFDY |
| 877 | P53 | DIQMTQSPSSLSASVGDRVTITCRASQDVNTAVAWYQQKPGKAP |
| | | KLLIYSAYFLYSGVPSRFSGSRSGTDFTLTISSLQPEDFATYYCQQY |
| | | SRYSPVTFGQGTKVEIKRTGGGSGGGGSGGGASEVQLVESGGGL |
| | | VQPGGSLRLSCAASGFNVYASGMHWVRQAPGKGLEWVAKIYPD |
| | | SDYTYYADSVKGRFTISADTSKNTAYLQMNSLRAEDTAVYYCSR |
| | | DSSFYYVYAMDYWGQGTLVTVSS |
| 878 | P53 | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQQ |
| | | SSTPVTFGQGTKVEIKRTGGGSGGGGSGGGASEVQLVESGGGLVQ |
| | | PGGSLRLSCAASGFNVYQSDMHWVRQAPGKGLEWVATIWPYSG |
| | | YTYYADSVKGRFTISADTSKNTAYLQMNSLRAEDTAVYYCSRDG |
| | | MYAFDYWGQGTLVTVSS |
| 879 | P53 | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | SYYPNTFGQGTKVEIKRTGGGSGGGGSGGGASEVQLVESGGGLV |
| | | QPGGSLRLSCAASGFNLYQRDMHWVRQAPGKGLEWVAGLLYGS |
| | | DHTEYADSVKGRFTISADTSKNTAYLQMNSLRAEDTAVYYCSRA |
| | | TYEEAFDYWGQGTLVTVSS |
| 880 | P53 | DIQMTQSPSSLSASVGDRVTITCRASQDVNTAVAWYQQKPGKAP |
| | | KLLIYSASFLYSGVPSRFSGSRSGTDFTLTITSLQPEDFATYYCQQQ |
| | | WSSPDTFGQGTKVEIKRTGGGSGGGGSGGGASEVQLVESGGGLV |
| | | QPGGSLRLSCAASGFNLSYYDMHWVRQAPGKGLEWVALIYYGS |
| | | GYTYYADSVKGRFTISADTSKNTAYLQMNSLRAEDTAVYYCSRG |
| | | SYVSGMDYWGQGTLVTVSS |
| 881 | P53 | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | NAYPITFGQGTKVEIKRTGGGSGGGGSGGGASEVQLVESGGGLV |
| | | QPGGSLRLSCAASGFNLNSYYMHWVRQAPGKGLEWVAMIIPGYG |
| | | YTNYADSVKGRFTISADTSKNTAYLQMNSLRAEDTAVYYCSRSY |
| | | YMYMDYWGQGTLVTVSS |
| 882 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQVI |
| | | YYPFTFGQGTKVEIKRTGGGSGGGGSGGGASEVQLVESGGGLVQ |
| | | PGGSLRLSCAASGFNLYSYAIHWVRQAPGKGLEWVALLYPDYGV |
| | | TSYADSVKGRFTISADTSKNTAYLQMNSLRAEDTAVYYCSRYRS |
| | | YEYSVSSYSYSAMDYWGQGTLVTVSS |
| 883 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQY |
| | | DYYPFTFGQGTKVEIKRTGGGSGGGGSGGGASEVQLVESGGGLV |
| | | QPGGSLRLSCAASGFNISYEAMHWVRQAPGKGLEWVALIYPNHG |
| | | ITSYADSVKGRFTISADTSKNTAYLQMNSLRAEDTAVYYCSRYSS |
| | | SAMDYWGQGTLVTVSS |
| 884 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQSI |
| | | YYPFTFGQGTKVEIKRTGGGSGGGGSGGGASEVQLVESGGGLVQ |
| | | PGGSLRLSCAASGFNLYTSQMHWVRQAPGKGLEWVALVYPGYY |
| | | VTSYADSVKGRFTISADTSKNTAYLQMNSLRAEDTAVYYCSRGA |
| | | YYYSSAMDYWGQGTLVTVSS |
| 885 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQY |
| | | DYYPFTFGQGTKVEIKRTGGGSGGGGSGGGASEVQLVESGGGLV |
| | | QPGGSLRLSCAASGFNVFGYAIHWVRQAPGKGLEWVAEVYPGY |
| | | DVTSYADSVKGRFTISADTSKNTAYLQMNSLRAEDTAVYYCSRY |
| | | SWAGAFDYWGQGTLVTVSS |
| 886 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | SYSPWTFGQGTKVEIKRTGGGSGGGGSGGGASEVQLVESGGGLV |
| | | QPGGSLRLSCAASGFNISPWDMHWVRQAPGKGLEWVAQLYPSSG |
| | | YTNYADSVKGRFTISADTSKNTAYLQMNSLRAEDTAVYYCSRSV |
| | | YWSLDYWGQGTLVTVSS |
| 887 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | FSTPITFGQGTKVEIKRTGGGSGGGGSGGGASEVQLVESGGGLVQ |
| | | PGGSLRLSCAASGFNISEYLMHWVRQAPGKGLEWVALLPPGLSY |
| | | TNYADSVKGRFTISADTSKNTAYLQMNSLRAEDTAVYYCSRYGY |
| | | YAFDYWGQGTLVTVSS |
| 888 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQG |
| | | EYSPLTFGQGTKVEIKRTGGGSGGGGSGGGASEVQLVESGGGLV |
| | | QPGGSLRLSCAASGFNVFESAMHWVRQAPGKGLEWVAWVYGSY |
| | | DYTYYADSVKGRFTISADTSKNTAYLQMNSLRAEDTAVYYCSRS |
| | | FAYFQAMDYWGQGTLVTVSS |
| 889 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQT |
| | | YYTPVTFGQGTKVEIKRTGGGSGGGGSGGGASEVQLVESGGGLV |
| | | QPGGSLRLSCAASGFNISHYVMHWVRQAPGKGLEWVADFYPHSD |
| | | STYYADSVKGRFTISADTSKNTAYLQMNSLRAEDTAVYYCSRYQ |
| | | SYSFDYWGQGTLVTVSS |
| 890 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQA |
| | | SRQPYTFGQGTKVEIKRTGGGSGGGGSGGGASEVQLVESGGGLV |
| | | QPGGSLRLSCAASGFNFSESGMHWVRQAPGKGLEWVAHFSGDSG |
| | | YTYYADSVKGRFTISADTSKNTAYLQMNSLRAEDTAVYYCSRYM |
| | | YYSGYFDYWGQGTLVTVSS |
| 891 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQA |
| | | VSYPWTFGQGTKVEIKRTGGGSGGGGSGGGASEVQLVESGGGLV |
| | | QPGGSLRLSCAASGFNISSSGIHWVRQAPGKGLEWVAMVYGGSG |
| | | YTNYADSVKGRFTISADTSKNTAYLQMNSLRAEDTAVYYCSRWA |
| | | HYSAYMDYWGQGTLVTVSS |
| 892 | RAS | DIQMTQSPSSLSASVGDRVTITCRASQDVNTANAWYQQKPGKAP |
| | | KLLIYSASFLYSGVPSRFSGSRSGTDFTLTISSLQPEDFATYYCQQT |
| | | SSYPITFGQGTKVEIKRTGGGSGGGGSGGGASEVQLVESGGGLVQ |
| | | PGGSLRLSCAASGFNIYWYGMHWVRQAPGKGJEWVAQVYPWSG |
| | | FTYYADSVKGRFTISADTSKNTAYLQMNSLRAEDTAVYYCSRDY |
| | | YSYSLDYWGQGTLVTVSS |
| 893 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | WYSPSTFGQGTKVEIKRTGGGSGGGGSGGGASEVQLVESGGGLV |
| | | QPGGSLRLSCAASGFNISASGMHWVRQAPGKGLEWVAWIWGGS |
| | | SYTYYADSVKGRFTISADTSKNTAYLQMNSLRAEDTAVYYCSRG |
| | | QYTSYMDYWGQGTLVTVSS |
| 894 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | YYAPITFGQGTKVEIKRTGGGSGGGGSGGGASEVQLVESGGGLV |
| | | QPGGSLRLSCAASGFNFSYYGMHWVRQAPGKGLEWVAWIYPFS |
| | | GYTNYADSVKGRFTISADTSKNTAYLQMNSLRAEDTAVYYCSRE |
| | | YYSRAFDYWGQGTLVTVSS |
| 895 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | YYSPWTFGQGTKVEIKRTGGGSGGGGSGGGASEVQLVESGGGLV |
| | | QPGGSLRLSCAASGENISYSNIHWVRQAPGKGLEWVAMIYGTRG |
| | | GTYYADSVKGRFTISADTSKNTAYLQMNSLRAEDTAVYYSCRYY |
| | | SYAMDYWGQGTLVTVSS |
| 896 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQA |
| | | YYPPWTFGQGTKVEIKRTGGGSGGGGSGGGASEVQLVESGGGLV |
| | | QPGGSLRLSCAASGFNVSRWAMHWVRQAPGKGLEWVARVYPSG |
| | | YLTYYADSVKGRFTISADTSKNTAYLQMNSLRAEDTAVYYCSRN |
| | | MQSYMDYWGQGTLVTVSS |
| 897 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | YSSFPVTFGQGTKVEIKRTGGGSGGGGSGGGASEVQLVESGGGLV |
| | | QPGGSLRLSCAASGFNFSYGGIHWVRQAPGKGLEWVAMIYPLTG |
| | | YTNYADSVKGRFTISADTSKNTAYLQMNSLRAEDTAVYYCSRDY |
| | | YYSVDVWGQGTLVTVSS |
| 898 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQT |
| | | YYYPFTFGQGTKVEIKRTGGGSGGGGSGGGASEVQLVESGGGLV |
| | | QPGGSLRLSCAASGFNLYAWGMHWVRQAPGKGLEWVALVYGG |
| | | WGSTSYADSVKGRFTISADTSKNTAYLQMNSLRAEDTAVYYCSR |
| | | AGSSKMSAGAFDYWGQGTLVTVSS |
| 899 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | YYPYYPWTFGQGTKVEIKRTGGGSGGGGSGGGASEVQLVESGGG |
| | | LVQPGGSLRLSCAASGFNVSHSAMHWVRQAPGKGLEWVATVHP |
| | | DWGNTYYADSVKGRFTISADTSKNTAYLQMNSLRAEDTAVYYSC |
| | | RWQQYYYSFDYWGQGTLVTVSS |
| 900 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQY |
| | | DRPITFGQGTKVEIKRTGGGSGGGGSGGGASEVQLVESGGGLVQP |
| | | GGSLRLSCAASGFNIYYEAMHWVRQAPGKGLWEVAQIYPWNDY |
| | | TYYADSVKGRFTISADTSKNTAYLQMNSLRAEDTAVYYCSRNYY |
| | | AATMDYWGQGTLVTVSS |
| 901 | RAS | DIQMTQSPSSLSASVGFRVTITCRASQDVNTAVAWYQQKPGKAPK |
| | | LLIYSASFLYSGVPSRFSGSRSGTDFTLTISSLQPEDFATYYCQQSY |
| | | TSPLTFGQGTKVEIKRTGGGSGGGGSGGGASEVQLVESGGGLVQP |
| | | GGSLRLSCAASGFNIYYGVMHWVRQAPGKGLEWVAMIYPDSSW |
| | | TYYADSVKGRFTISADTSKNTAYLQMNSLRAEDTAVYYCSRDQD |
| | | FHYMNYYLSYALDYWGQGTLVTVSS |
| 902 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQY |
| | | WYYYPITFGQGTKVEIKRTGGGSGGGGSGGGASEVQLVESGGGL |
| | | VQPGGSLRLSCAASGFNITSTDMHWVRQAPGKGLEWVAISPGGS |
| | | YTYYADSVKGRFTISADTSKNTAYLQMNSLRAEDTAVYYCSRSA |
| | | FTGYFDVWGQGTLVTVSS |
| 903 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQS |
| | | YYAPITFGQGTKVEIKRTGGGSGGGGSGGGASEVQLVESGGGLV |
| | | QPGGSLRLSACCSGFNVQWSHMHWVRQAPGKGLEWVARLSPPS |
| | | GYTNYADSVKGRFTISADTSKNTAYLQMNSLRAEDTAVYYCSRLI |
| | | LSKGGYGWAMDYWGQGTLVTVSS |
| 904 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQY |
| | | YLYQPITFGQGTKVEIKRTGGGSGGGGSGGGASEVQLVESGGGLV |
| | | QPGGSLRLSCAASGFNIGMYTMHWVRQAPGKGLEWVALVYPDS |
| | | GYTNYADSVKGRFTISADTSKNTAYLQMNSLRAEDTAVYYCSRY |
| | | TWQSMDYWGQGTLVTVSS |
| 905 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQY |
| | | SNYPLTFGQGTKVEIKRTGGGSGGGGSGGGASEVQLVESGGGLV |
| | | QPGGSLRLSCAASGFNVFYGSMHWVRQAPGKGLEWVAFIGPDST |
| | | YTYYADSVKGRFTISADTSKNTAYLQMNSLRAEDTAVYYCSRDL |
| | | GSAYAMDYWGQGTLVTVSS |
| 906 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQY |
| | | ASDPITFGQGTKVEIKRTGGGSGGGGSGGGASEVQLVESGGGLVQ |
| | | PGGSLRLSCAASHFNLDYGWMHWVRQAPGKGLEWVAWVVPGS |
| | | DYTDYADSVKGRFTISADTSKNTAYLQMNSLRAEDTAVYYCSRF |
| | | HYTAFDVWGQGTLVTVSS |
| 907 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQY |
| | | SYDPITFGQGTKVEIKRTGGGSGGGGSGGGASEVQLVESGGGLVQ |
| | | PGGSLRLSCAASGFNFSYSAMHWVRQAPGKGLEWVADVVPDGD |
| | | WTYYADSVKGRFTISADTSKNTAYLQMNSLRAEDTAVYYCSRG |
| | | WYALDYWGQGTLVTVSS |
| 908 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQTI |
| | | TDPVTFGQGTKVEIKRTGGGSGGGGSGGGASEVQLVESGGGLVQ |
| | | PGGSLRLSCAASGFNVDWAWMHWVRQAPGKGLEWVAWVVGG |
| | | SDYTYYADSVKGRFTISADTSKNTAYLQMNSLRAEDTAVYYCSR |
| | | SYYYAFDYWGQGTLVTVSS |
| 909 | RAS | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQL |
| | | MYDPITFGQGTKVEIKRTGGGSGGGGSGGGASEVQLVESGGGLV |
| | | QPGGSLRLSCAASGFNFGTYWMHWVRQAPGKGLEWVAWFLPD |
| | | YDYTLYADSVKGRFTISADTSKNTAYLQMNSLRAEDTAVYYCSR |
| | | HGEYAFDYWGQGTLVTVSS |
| 910 | RAS | MTEYKLVVVGAGGVGKSALTIQLIQNHFVDEYDPTIEDSYRKQVV |
| | | IDGETCLLDILDTAGQEEYSAMRDQYMRTGEGFLCVFAINNTKSF |
| | | EDIHHYREQIKRVKDSEDVPMVLVGNKCDLPSRTVDTKQAQDLA |
| | | RSYGIPFIETSAKTRQRVEDAFYTLVREIRQYRLKKISKEEKTPGCV |
| | | KIKKCIIM |
| 911 | PIK3CA | MPPRPSSGELWGIHLMPPRILVECLLPNGMIVTLECLREATLITIKH |
| | | ELFKEARKYPLHQLLQDESSYIFVSVTQEAEREEFFDETRRLCDLR |
| | | LFQPFLKVIEPVGNREEKILNREIGFAIGMPVCEFDMVKDPEVQDF |
| | | RRNILNVCKEAVDLRDLNSPHSRAMYVYPPNVESSPELPKHIYNK |
| | | LDKGQIIVVIWVIVSPNNDKQKYTLKINHDCVPEQVIAEAIRKKTR |
| | | SMLLSSEQLKLCVLEYQGKYILKVCGCDEYFLEKYPLSQYKYIRS |
| | | CIMLGRMPNLMLMAKESLYSQLPMDCFTMPSYSRRISTATPYMN |
| | | GETSTKSLWVINSALRIKILCATYVNVNIRDIDKIYVRTGIYHGGEP |
| | | LCDNVNTQRVPCSNPRWNEWLNYDIYIPDLPRAARLCLSICSVKG |
| | | RKGAKEEHCPLAWGNINLFDYTDTLVSGKMALNLWPVPHGLEDL |
| | | LNPIGVTGSNPNKETPCLELEFDWFSSVVKFPDMSVIEEHANWSVS |
| | | REAGFSYSHAGLSNRLARDNELRENDKEQLKAISTRDPLSEIEQEK |
| | | DFLWSHRHYCVTIPEILPKLLLSVKWNSRDEVAQMYCLVKDWPPI |
| | | KPEQAMELLDCNYPDPMVRGFAVRCLEKYLTDDKLSQYLIQLVQ |
| | | VLKYEQYLDNLLVRFLLKKALTNQRIGHFFFWHLKSEMHNKTVS |
| | | QRFGLLLESYCRACGMYLKHLNRQVEAMEKLINLTDILKQEKKD |
| | | ETQKVQMKFLVEQMRRPDFMDALQGFLSPLNPAHQLGNLRLEEC |
| | | RIMSSAKRPLWLNWENPDIMSELLFQNNEIIFKNGDDLRQDMLTL |
| | | QIIRIMENIWQNQGLDLRMLPYGCLSIGDCVGLIEVVRNSHTIMQI |
| | | QCKGGLKGALQFNSHTLHQWLKDKNKGEIYDAAIDLFTRSCAGY |
| | | CVATFILGIGDRHNSNIMVKDDGQLFHIDFGHFLDHKKKKFGYKR |
| | | ERVPFVLTQDFLIVISKGAQECTKTREFERFQEMCYKAYLAIRQHA |
| | | NLFINLFSMMLGSGMPELQSFDDIAYIRKTLALDKTEQEALEYFM |
| | | KQMNDAHHGGWTTKMDWIFHTIKQHALN |
| 912 | P53 | MEEPQSDPSVEPPLSQETFSDLWKLLPENNVLSPLPSQAMDDLML |
| | | SPDDIEQWFIEDPGPDEAPRMPEAAPPVAPAPAAPTPAAPAPAPSW |
| | | PLSSSVPSQKTYQGSYGFRLGFLHSGTAKSVTCTYSPALNKMFCQ |
| | | LAKTCPVQLWVDSTPPPGTRVRAMAIYKQSQHMTEVVRRCPHHE |
| | | RCSDSDGLAPPQHLIRVEGNLRVEYLDDRNTFRHSVVVPYEPPEV |
| | | GSDCTTIHYNYMCNSSCMGGMNRRPILTIITLEDSSGNLLGRNSFE |
| | | VRVCACPGRDRRIFEENLRKKGEPHHELPPGSTKRALPNNTSSSPQ |
| | | PKKKPLDGEYFTLQIRGRERFEMFRELNEALELKDAQAGKEPGGS |
| | | RAHSSHLKSKKGQSTSRHKKLMFKIEGPDSD |
| 913 | RAS | SARDRGLVSLPSVEAFF |
| 914 | RAS | CAGRNFGNEKLTF |
| 915 | RAS | MLLLLLLLGPGSGLGAVVSQHPSRVICKSGTSVKIECRSLDFQATT |
| | | MFWYRQFPKQSLMLMATSNEGSKATYEQGVEKDKFLINHASLTL |
| | | STLTVTSAHPEDSSFYICSARDRGLVSLPSVEAFFGQGTRLTVVLE |
| | | DLNKVFPPEVAVFEPSEAEISHTQKATLVCLATGFFPDHVELSWW |
| | | VNGKEVHSGVSTDPQPLKEQPALNDSRYCLSSRLRVSATFWQNPR |
| | | NHFRCQVQFYGLSENDEWTQDRAKPVTQIVSAEAWGRADCGFTS |
| | | VSYQQGVLSATILYEILLGKATLYAVLVSALVLMAMVKRKDFGG |
| | | SG |
| 916 | RAS | MLLEHLLIILWMQLTWVSGQQLNQSPQSMFIQEGEDVSMNCTSSS |
| | | IFNTWLWYKQDPGEGPVLLIALYKAGELTSNGRLTAQFGITRKDS |
| | | FLNISASIPSDVGIYFCAGRNFGNEKLTFGTGTRLTIIPDIQNPDPAV |
| | | YQLRDSKSSDKSVCLFTDFDSQTNVSQSKDSDVYITDKCVLDMRS |
| | | MDFKSNSAVAWSNKSDFACANAFNNSIIPEDTFFPSPESSCDVKLV |
| | | EKSFETDTNLNFQNLSVIGFRILLLKVAGFNLLMTLRLWSS* |
| 917 | RAS | ASYLSGSIYNEQFF |
| 918 | RAS | CAVRDQSGANNLFF |
| 919 | RAS | MGTSLLCWMALCLLGADHADTGVSQDPRHKITKRGQNVTFRCD |
| | | PISEHNRLYWYRQTLGQGPEFLTYFQNEAQLEKSRLLSDRFSAERP |
| | | KGSFSTLEIQRTEQGDSAMYLCASYLSGSIYNEQFFGPGTRLTVLE |
| | | DLNKVFPPEVAVFEPSEAEISHTQKATLVCLATGFFPDHVELSWW |
| | | VNGKEVHSGVSTDPQPLKEQPALNDSRYCLSSRLRVSATFWQNPR |
| | | NHFRCQVQFYGLSENDEWTQDRAKPVTQIVSAEAWGRADCGFTS |
| | | VSYQQGVLSATILYEILLGKATLYAVLVSALVLMAMVKRKDFGG |
| | | SG |
| 920 | RAS | MASAPISMLAMLFTLSGLRAQSVAQPEDQVNVAEGNPLTVKCTY |
| | | SVSGNPYLFWYVQYPNRGLQFLLKYITGDNLVKGSYGFEAEFNKS |
| | | QTSFHLKKPSALVSDSALYFCAVRDQSGANNLFFGTGTRLTVIPDI |
| | | QNPDPAVYQLRDSKSSDKSVCLFTDFDSQTNVSQSKDSDVYITDK |
| | | TVLDMRSMDFKSNSAVAWSNKSDFACANAFNNSIIPEDTFFPSPES |
| | | SCDVKLVEKSFETDTNLNFQNLSVIGFRILLLKVAGFNLLMTLRL |
| | | WSS |
| 921 | RAS | ASSYSTERGTIY |
| 922 | RAS | AASGGGGADGLT |
| 923 | RAS | MRSQNDFLESPVPLSSMHRYRRPLRPGAPAMSISLLCCAAFPLLW |
| | | AGPVNAGVTQTPKFRILKIGQSMTLQCTQDMNHNYMYWYRQDP |
| | | GMGLKLIYYSVGAGITDKGEVPNGYNVSRSTTEDFPLRLELAAPS |
| | | QTSVYFCASSYSTERGTIYFGEGSWLTVVEDLNKVFPPEVAVFEPS |
| | | EAEISHTQKATLVCLATGFFPDHVELSWWVNGKEVHSGVSTDPQ |
| | | PLKEQPALNDSRYCLSSRLRVSATFWQNPRNHFRCQVQFYGLSEN |
| | | DEWTQDRAKPVTQIVSAEAWGRADCGFTSVSYQQGVLSATILYEI |
| | | LLGKATLYAVLVSALVLMAMVKRKDF |
| 924 | RAS | MAMLLGASVLILWLQPDWVNSQQKNDDQQVKQNSPSLSVQEGR |
| | | ISILNCDYTNSMFDYFLWYKKYPAEGPTFLISISSIKDKNEDGRFTV |
| | | FLNKSAKHLSLHIVPSQPGDSAVYFCAASGGGGADGLTFGKGTHL |
| | | IIQPDIQNPDPAVYQLRDSKSSDKSVCLFTDFDSQTNVSQSKDSDV |
| | | YITDKTVLDMRSMDFKSNSAVAWSNKSDFACANAFNNSIIPEDTF |
| | | FPSPESSCDVKLVEKSFETDTNLNFQNLSVIGFRILLLKVAGENLL |
| | | MTLRLWSS |
| 925 | RAS | ASSLADIYEQY |
| 926 | RAS | ATDRQSSGDKLT |
| 927 | RAS | MGTRLLCWAALCLLGAELTEAGVAQSPRYKIIEKRQSVAFWCNPI |
| | | SGHATLYWYQQILGQGPKLLIQFQNNGVVDDSQLPKDRFSAERL |
| | | KGVDSTLKIQPAKLEDSAVYLCASSLADIYEQYFGPGTRLTVTEDL |
| | | NKVFPPEVAVFEPSEAEISHTQKATLVCLATGFFPDHVELSWWVN |
| | | GKEVHSGVSTDPQPLKEQPALNDSRYCLSSRLRVSATFWQNPRNH |
| | | FRCQVQFYGLSENDEWTQDRAKPVTQIVSAEAWGRADCGFTSVS |
| | | YQQGVLSATILYEILLGKATLYAVLVSALVLMAMVKRKDF |
| 928 | RAS | METLLGVSLVILWLQLARVNSQQGEEDPQALSIQEGENATMNCSY |
| | | KTSINNLQWYRQNSGRGLVHLILIRSNEREKHSGRLRVTLDTSKKS |
| | | SSLLITASRAADTASYFCATDRQSSGDKLTFGTGTRLAVRPDIQNP |
| | | DPAVYQLRDSKSSDKSVCLFTDFDSQTNVSQSKDSDVYITDKTVL |
| | | DMRSMDFKSNSAVAWSNKSDFACANAFNNSIIPEDTFFPSPESSCD |
| | | VKLVEKSFETDTNLNFQNLSVIGFRILLLKVAGFNLLMTLRLWSS |
| 929 | RAS | CASSARNDEAFF |
| 930 | RAS | CAPGDNFNKFYF |
| 931 | RAS | MGSRLLCWVLLCLLGAGPVKAGVTQTPRYLIKTRGQQVTLSCSPI |
| | | SGHRSVSWYQQTPGQGLQFLFEYFSETQRNKGNFPGRFSGRQFSN |
| | | SRSEMNVSTLELGDSALYLCASSARNDEAFFGQGTRLTVVEDLNK |
| | | VFPPEVAVFEPSEAEISHTQKATLVCLATGFFPDHVELSWWVNGK |
| | | EVHSGVSTDPQPLKEQPALNDSRYCLSSRLRVSATFWQNPRNHFR |
| | | CQVQFYGLSENDEWTQDRAKPVTQIVSAEAWGRADCGFTSVSYQ |
| | | QGVLSATILYEILLGKATLYAVLVSALVLMAMVKRKDF |
| 932 | RAS | MWGVFLLYVSMKMGGTTGQNIDQPTEMTATEGAIVQINCTYQTS |
| | | GFNGLFWYQQHAGEAPTFLSYNVLDGLEEKGRFSSFLSRSKGYSY |
| | | LLLKELQMKDSASYLCAPGDNFNKFYFGSGTKLNVKPDIQNPDPA |
| | | VYQLRDSKSSDKSVCLFTDFDSQTNVSQSKDSDVYITDKTVLDMR |
| | | SMDFKSNSAVAWSNKSDFACANAFNNSIIPEDTFFPSPESSCDVKL |
| | | VEKSFETDTNLNFQNLSVIGFRILLLKVAGFNLLMTLRLWSS |
| 933 | RAS | CASSLGDSEQYF |
| 934 | RAS | CAVKSRAGSYQLTF |
| 935 | RAS | MDSWTFCCVSLCILVAKHTDAGVIQSPRHEVTEMGQEVTLRCKPI |
| | | SGHNSLFWYRQTMMRGLELLIYFNNNVPIDDSGMPEDRFSAKMP |
| | | NASFSTLKIQPSEPRDSAVYFCASSLGDSEQYFGPGTRLTVTEDLN |
| | | KVFPPEVAVFEPSEAEISHTQKATLVCLATGFFPDHVELSWWVNG |
| | | KEVHSGVSTDPQPLKEQPALNDSRYCLSSRLRVSATFWQNPRNHF |
| | | RCQVQFYGLSENDEWTQDRAKPVTQIVSAEAWGRADCGFTSVSY |
| | | QQGVLSATILYEILLGKATLYAVLVSALVLMAMVKRKDF |
| 936 | RAS | MKSLRVLLVILWLQLSWVWSQQKEVEQNSGPLSVPEGAIASLNCT |
| | | YSDRGSQSFFWYRQYSGKSPELIMFIYSNGDKEDGRFTAQLNKAS |
| | | QYVSLLIRDSQPSDSATYLCAVKSRAGSYQLTFGKGTKLSVIPDIQ |
| | | NPDPAVYQLRDSKSSDKSVCLFTDFDSQTNVSQSKDSDVYITDKT |
| | | VLDMRSMDFKSNSAVAWSNKSDFACANAFNNSIIPEDTFFPSPESS |
| | | CDVKLVEKSFETDTNLNFQNLSVIGFRILLLKVAGFNLLMTLRLW |
| | | SS |
| 937 | RAS | CASSQRSNTGELFF |
| 938 | RAS | CVVSGGGSSNTGKLIF |
| 939 | RAS | MGTRLLCWAALCLLGAELTEAGVAQSPRYKIIEKRQSVAFWCNPI |
| | | SGHATLYWYQQILGQGPKLLIQFQNNGVVDDSQLPKDRFSAERL |
| | | KGVDSTLKIQPAKLEDSAVYLCASSQRSNTGELFFGEGSRLTVLED |
| | | LNKVFPPEVAVFEPSEAEISHTQKATLVCLATGFFPDHVELSWWV |
| | | NGKEVHSGVSTDPQPLKEQPALNDSRYCLSSRLRVSATFWQNPRN |
| | | HFRCQVQFYGLSENDEWTQDRAKPVTQIVSAEAWGRADCGFTSV |
| | | SYQQGVLSATILYEILLGKATLYAVLVSALVLMAMVKRKDFGGS |
| | | G |
| 940 | RAS | MLLLLVPVLEVIFTLGGTRAQSVTQLDSHVSVSEGTPVLLRCNYSS |
| | | SYSPSLFWYVQHPNKGLQLLLKYTSAATLVKGINGFEAEFKKSET |
| | | SFHLTKPSAHMSDAAEYFCVVSGGGSSNTGKLIFGQGTTLQVKPD |
| | | IQNPDPAVYQLRDSKSSDKSVCLFTDFDSQTNVSQSKDSDVYITD |
| | | KTVLDMRSMDFKSNSAVAWSNKSDFACANAFNNSIIPEDTFFPSP |
| | | ESSCDVKLVEKSFETDTNLNFQNLSVIGFRILLLKVAGFNLLMTLR |
| | | LWSS |
| 941 | RAS | CASGGRDSTDTQYF |
| 942 | RAS | CATDAGGGADGLTF |
| 943 | RAS | MGFRLLCCVAFCLLGAGPVDSGVTQTPKHLITATGQRVTLRCSPR |
| | | SGDLSVYWYQQSLDQGLQFLIQYYNGEERAKGNILERFSAQQFPD |
| | | LHSELNLSSLELGDSALYFCASGGRDSTDTQYFGPGTRLTVLEDL |
| | | NKVFPPEVAVFEPSEAEISHTQKATLVCLATGFFPDHVELSWWVN |
| | | GKEVHSGVSTDPQPLKEQPALNDSRYCLSSRLRVSATFWQNPRNH |
| | | FRCQVQFYGLSENDEWTQDRAKPVTQIVSAEAWGRADCGFTSVS |
| | | YQQGVLSATILYEILLGKATLYAVLVSALVLMAMVKRKDF |
| 944 | RAS | METLLGVSLVILWLQLARVNSQQGEEDPQALSIQEGENATMNCSY |
| | | KTSINNLQWYRQNSGRGLVHLILIRSNEREKHSGRLRVTLDTSKKS |
| | | SSLLITASRAADTASYFCATDAGGGADGLTFGKGTHLIIQPDIQNP |
| | | DPAVYQLRDSKSSDKSVCLFTDFDSQTNVSQSKDSDVYITDKTVL |
| | | DMRSMDFKSNSAVAWSNKSDFACANAFNNSIIPEDTFFPSPESSCD |
| | | VKLVEKSFETDTNLNFQNLSVIGFRILLLKVAGFNLLMTLRLWSS |
| 945 | RAS | ASSTSFWEVNTEAF |
| 946 | RAS | AGGPNTGNQFY |
| 947 | RAS | MGPGLLCWVLLCLLGAGSVETGVTQSPTALIKTRGQQVTLRCSSQ |
| | | SGHNTVSWYQQALGQGPQFIFQYYREEENGRGNFPPRFSGLQFPN |
| | | YSSELNVNALELDDSALYLCASSTSFWEVNTEAFFGQGTRLTVVK |
| | | DLRNVTPPKVSLFEPSKAEIANKQKATLVCLARGFFPDHVELSWW |
| | | VNGKEVHSGVSTDPQAYKESNYSYCLSSRLRVSATFWHNPRNHF |
| | | RCQVQFHGLSEEDKWPEGSPKPVTQNISAEAWGRADCGITSASYH |
| | | QGVLSATILYEILLGKATLYAVLVSGLVLMAMVKRKDSRGGGSG |
| 948 | RAS | MVLKFSVSILWIQLAWVSTQLLEQSPQFLSIQEGENLTVYCNSSSV |
| | | FSSLQWYRQEPGEGPVLLVTVVTGGEVKKLKRLTFQFGDARKDS |
| | | SLHITAAQPGDTGLYLCAGGPNTGNQFYFGTGTSLTVIPNDIQNPE |
| | | PAVYQLKDPRSQDSTLCLFTDFDSQINVPKTMESGTFITDKTVLD |
| | | MKAMDSKSNGAIAWSNQTSFTCQDIFKETNATYPSSDVPCDATLT |
| | | EKSFETDMNLNFQNLSVMGLRILLLKVAGFNLLMTLRLWSS |
| 949 | RAS | ASSKRGWPYEQY |
| 950 | RAS | AVREEVLYNQGGKLI |
| 951 | RAS | MSNQVLCCVVLCLLGANTVDGGITQSPKYLFRKEGQNVTLSCEQ |
| | | NLNHDAMYWYRQDPGQGLRLIYYSQIVNDFQKGDIAEGYSVSRE |
| | | KKESFPLTVTSAQKNPTAFYLCASSKRGWPYEQYFGPGTRLTVTK |
| | | DLRNVTPPKVSLFEPSKAEIANKQKATLVCLARGFFPDHVELSWW |
| | | VNGKEVHSGVCTDPQAYKESNYSYCLSSRLRVSATFWHNPRNHF |
| | | RCQVQFHGLSEEDKWPEGSPKPVTQNISAEAWGRADCGITSASYH |
| | | QGVLSATILYEILLGKATLYAVLVSGLVLMAMVKRKDSRGGGSG |
| 952 | RAS | MWGVFLLYVSMKMGGTTGQNIDQPTEMTATEGAIVQINCTYQTS |
| | | GFNGLFWYQQHAGEAPTFLSYNVLDGLEEKGRFSSFLSRSKGYSY |
| | | LLLKELQMKDSASYLCAVREEVLYNQGGKLIFGQGTELSVKPDIQ |
| | | NPEPAVYQLKDPRSQDSTLCLFTDFDSQINVPKTMESGTFITDKCV |
| | | LDMKAMDSKSNGAIAWSNQTSFTCQDIFKETNATYPSSDVPCDAT |
| | | LTEKSFETDMNLNFQNLSVMGLRILLLKVAGFNLLMTLRLWSS |
| 953 | RAS | ASSLADIYEQY |
| 954 | RAS | ATDRQSSGDKLT |
| 955 | RAS | MGTRLLCWAALCLLGAELTEAGVAQSPRYKIIEKRQSVAFWCNPI |
| | | SGHATLYWYQQILGQGPKLLIQFQNNGVVDDSQLPKDRFSAERL |
| | | KGVDSTLKIQPAKLEDSAVYLCASSLADIYEQYFGPGTRLTVTKD |
| | | LRNVTPPKVSLFEPSKAEIANKQKATLVCLARGFFPDHVELSWWV |
| | | NGKEVHSGVCTDPQAYKESNYSYCLSSRLRVSATFWHNPRNHFR |
| | | CQVQFHGLSEEDKWPEGSPKPVTQNISAEAWGRADCGITSASYHQ |
| | | GVLSATILYEILLGKATLYAVLVSGLVLMAMVKKKNS |
| 956 | RAS | METLLGVSLVILWLQLARVNSQQGEEDPQALSIQEGENATMNCSY |
| | | KTSINNLQWYRQNSGRGLVHLILIRSNEREKHSGRLRVTLDTSKKS |
| | | SSLLITASRAADTASYFCATDRQSSGDKLTFGTGTRLAVRPDIQNP |
| | | EPAVYQLKDPRSQDSTLCLFTDFDSQINVPKTMESGTFITDKCVLD |
| | | MKAMDSKSNGAIAWSNQTSFTCQDIFKETNATYPSSDVPCDATLT |
| | | EKSFETDMNLNFQNLSVMGLRILLLKVAGFNLLMTLRLWSS* |
| 957 | RAS | ASSSTDRIEAF |
| 958 | RAS | ALYIYGGSQGNLI |
| 959 | RAS | ASTTFKTGRAIEKLF |
| 960 | RAS | ATYNFNKFY |
| 961 | RAS | ASSSRGHSGTEAF |
| 962 | RAS | AVRDRGGSYIPT |
| 963 | RAS | ASSSRGHSGTEAF |
| 964 | RAS | AGLYSSASKII |
| 965 | RAS | ATYKVGDEQF |
| 966 | RAS | LANTGGFKTI |
| 967 | RAS | ASSDWLAGAKDEQY |
| 968 | RAS | AETGFQKLV |
| 969 | RAS | MGIRLLCRVAFCFLAVGLVDVKVTQSSRYLVKRTGEKVFLECVQ |
| | | DMDHENMFWYRQDPGLGLRLIYFSYDVKMKEKGDIPEGYSVSRE |
| | | KKERFSLILESASTNQTSMYLCASSDWLAGAKDEQYFGPGTRLTV |
| | | TKDLRNVTPPKVSLFEPSKAEIANKQKATLVCLARGFFPDHVELS |
| | | WWVNGKEVHSGVCTDPQAYKESNYSYCLSSRLRVSATFWHNPR |
| | | NHFRCQVQFHGLSEEDKWPEGSPKPVTQNISAEAWGRADCGITSA |
| | | SYHQGVLSATILYEILLGKATLYAVLVSGLVLMAMVKRKDFGGS |
| | | G |
| 970 | RAS | MTSIRAVFIFLWLQLDLVNGENVEQHPSTLSVQEGDSAVIKCTYS |
| | | DSASNYFPWYKQELGKRPQLIIDIRSNVGEKKDQRIAVTLNKTAK |
| | | HFSLHITETQPEDSAVYFCAETGFQKLVFGTGTRLLVSPDIQNPEP |
| | | AVYQLKDPRSQDSTLCLFTDFDSQINVPKTMESGTFITDKCVLDM |
| | | KAMDSKSNGAIAWSNQTSFTCQDIFKETNATYPSSDVPCDATLTE |
| | | KSFETDMNLNFQNLSVMGLRILLLKVAGFNLLMTLRLWSS |
| 971 | RAS | ASSLVASNEQF |
| 972 | RAS | ATDPLDYKLS |
| 973 | RAS | ASSLGLLLYNEQF |
| 974 | RAS | ACQGGSEKLV |
| 975 | RAS | ASSLGDSYEQYF |
| 976 | RAS | ATDAQTGANNLF |
| 979 | RAS | ASSEWGSTGELF |
| 978 | RAS | AANAGGTSYGKLT |
| 979 | RAS | ASSEWGSTGELF |
| 980 | RAS | AVDIIGGKST |
| 981 | RAS | ASSEYTMGTQY |
| 982 | RAS | AASAVGQEYGNKLV |
| 983 | RAS | MSNTAFPDPAWNTTLLSWVALFLLGTSSANSGVVQSPRYIIKGKG |
| | | ERSILKCIPISGHLSVAWYQQTQGQELKFFIQHYDKMERDKGNLPS |
| | | RFSVQQFDDYHSEMNMSALELEDSAVYFCASSLTDPLDSDYTFGS |
| | | GTRLLVIEDLRNVTPPKVSLFEPSKAEIANKQKATLVCLARGFFPD |
| | | HVELSWWVNGKEVHSGVCTDPQAYKESNYSYCLSSRLRVSATF |
| | | WHNPRNHFRCQVQFHGLSEEDKWPEGSPKPVTQNISAEAWGRAD |
| | | CGITSASYQQGVLSATILYEILLGKATLYAVLVSTLVVMAMVKRK |
| | | DSRGGGSG |
| 984 | RAS | MQRNLGAVLGILWVQICWVRGDQVEQSPSALSLHEGTDSALRCN |
| | | FTTTMRSVQWFRQNSRGSLISLFYLASGTKENGRLKSAFDSKERR |
| | | YSTLHIRDAQLEDSGTYFCAADSSNTGYQNFYFGKGTSLTVIPNIQ |
| | | NPEPAVYQLKDPRSQDSTLCLFTDFDSQINVPKTMESGTFITDKTV |
| | | LDMKAMDSKSNGAIAWSNQTSFTCQDIFKETNATYPSSDVPCDAT |
| | | LTEKSFETDMNLNFQNLSVMGLRILLLKVAGFNLLMTLRLWSS |
| 985 | RAS | CASSYGPGQHNSPLHF |
| 986 | RAS | CALSGPSGAGSYQLTF |
| 987 | RAS | MSLGLLCCGAFSLLWAGPVNAGVTQTPKFRVLKTGQSMTLLCAQ |
| | | DMNHEYMYWYRQDPGMGLRLIHYSVGEGTTAKGEVPDGYNVS |
| | | RLKKQNFLLGLESAAPSQTSVYFCASSYGPGQHNSPLHFGNGTRL |
| | | TVTEDLNKVFPPEVAVFEPSEAEISHTQKATLVCLATGFYPDHVEL |
| | | SWWVNGKEVHSGVCTDPQPLKEQPALNDSRYALSSRLRVSATFW |
| | | QDPRNHFRCQVQFYGLSENDEWTQDRAKPVTQIVSAEAWGRAD |
| | | CGFTSVSYQQGVLSATILYEILLGKATLYAVLVSALVLMAMVKRK |
| | | DFGGSG |
| 988 | RAS | MLTASLLRAVIASICVVSSMAQKVTQAQTEISVVEKEDVTLDCVY |
| | | ETRDTTYYLFWYKQPPSGELVFLIRRNSFDEQNEISGRYSWNFQKS |
| | | TSSFNFTITASQVVDSAVYFCALSGPSGAGSYQLTFGKGTKLSVIP |
| | | NIQNPDPAVYQLRDSKSSDKSVCLFTDFDSQTNVSQSKDSVDYIT |
| | | DKCVLDMRSMDFKSNSAVAWSNKSDFACANAFNNSIIPEDTFFPS |
| | | PESSCDVKLVEKSFETDTNLNFQNLSVIGFRILLLKVAGFNLLMTL |
| | | RLWSS |
| 989 | RAS | CASSVAGGGQETQY |
| 990 | RAS | CALSEAGTYKYIF |
| 991 | RAS | MGFRLLCCVAFCLLGAGPVDSGVTQTPKHLITATGQRVTLRCSPR |
| | | SGDLSVYWYQQSLDQGLQFLIQYYNGEERAKGNILERFSAQQFPD |
| | | LHSELNLSSLELGDSALYFCASSVAGGGQETQYFGPGTRLLVLED |
| | | LKNVFPPEVAVFEPSEAEISHTQKATLVCLATGFYPDHVELSWWV |
| | | NGKEVHSGVCTDPQPLKEQPALNDSRYCLSSRLRVSATFWQNPR |
| | | NHFRCQVQFYGLSENDEWTQDRAKPVTQIVSAEAWGRADCGFTS |
| | | ESYQQGVLSATILYEILLGKATLYAVLVSALVLMAMVKRKDSRG |
| | | GGSG |
| 992 | RAS | MLTASLLRAVIASICVVSSMAQKVTQAQTEISVVEKEDVTLDCVY |
| | | ETRDTTYYLFWYKQPPSGELVFLIRRNSFDEQNEISGRYSWNFQKS |
| | | TSSFNFTITASQVVDSAVYFCALSEAGTYKYIFGTGTRLKVLANIQ |
| | | NPDPAVYQLRDSKSSDKSVCLFTDFDSQTNVSQSKDSDVYITDKC |
| | | VLDMRSMDFKSNSAVAWSNKSDFACANAFNNSIIPEDTFFPSPESS |
| | | CDVKLVEKSFETDTNLNFQNLSVIGFRILLLKVAGFNLLMTLRLW |
| | | SS |
| 993 | RAS | CASSLSFRQGLREQYF |
| 994 | RAS | CAVNPPDTGFQKLVF |
| 995 | RAS | MGIRLLCRVAFCFLAVGLVDVKVTQSSRYLVKRTGEKVFLECVQ |
| | | DMDHENMFWYRQDPGLGLRLIYFSYDVKMKEKGDIPEGYSVSRE |
| | | KKERFSLILESASTNQTSMYLCASSLSFRQGLREQYFGPGTRLTVT |
| | | EDLKNVFPPEVAVFEPSEAEISHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | GGGSG |
| 996 | RAS | MISLRVLLVILWLQLSWVWSQRKEVEQDPGPFNVPEGATVAFNC |
| | | TYSNSASQSFFWYRQDCRKEPKLLMSVYSSGNEDGRFTAQLNRA |
| | | SQYISLLIRDSKLSDSATYLCAVNPPDTGFQKLVFGTGTRLLVSPNI |
| | | QNPDPAVYQLRDSKSSDKSVCLFTDFDSQTNVSQSKDSDVYITDK |
| | | CVLDMRSMDFKSNSAVAWSNKSDFACANAFNNSIIPEDTFFPSPES |
| | | SCDVKLVEKSFETDTNLNFQNLSVIGFRILLLKVAGFNLLMTLRL |
| | | WSS |
| 997 | RAS | CASKVYGYTF |
| 998 | RAS | CLVGDFNSNSGYALNF |
| 999 | RAS | MGTRLLFWVAFCLLGAYHTGAGVSQSPSNKVTEKGKDVELRCDP |
| | | ISGHTALYWYRQRLGQGLEFLIYFQGNSAPDKSGLPSDRFSAERT |
| | | GESVSTLTIQRTQQEDSAVYLCASKVYGYTFGSGTRLTVVEDLNK |
| | | VFPPEVAVFEPSEAEISHTQKATLVCLATGFFPDHVELSWWVNGK |
| | | EVHSGVCTDPQPLKEQPALNDSRYCLSSRLRVSATFWQNPRNHFR |
| | | CQVQFYGLSENDEWTQDRAKPVTQIVSAEAWGRADCGFTSVSYQ |
| | | QGVLSATILYEILLGKATLYAVLVSALVLMAMVKRKDFGGSG |
| 1000 | RAS | MRQVARVIVFLTLSTLSLAKTTQPISMDSYEGQEVNITCSHNNIAT |
| | | NDYITWYQQFPSQGPRFIIQGYKTKVTNEVASLFIPADRKSSTLSLP |
| | | RVSLSDTAVYYCLVGDFNSNSGYALNFGKGTSLLVTPHIQNPDPA |
| | | VYQLRDSKSSDKSVCLFTDFDSQTNVSQSKDSDVYITDKCVLDMR |
| | | SMDFKSNSAVAWSNKSDFACANAFNNSIIPEDTFFPSPESSCDVKL |
| | | VEKSFETDTNLNFQNLSVIGFRILLLKVAGFNLLMTLRLWSS |
| 1001 | RAS | CSASPRAGQLSSYNSPLHF |
| 1002 | RAS | CAVDKDGGYQKVTF |
| 1003 | RAS | MLLLLLLLGPGISLLLPGSLAGSGLGAVVSQHPSWVICKSGTSVKI |
| | | ECRSLDFQATTMFWYRQFPKQSLMLMATSNEGSKATYEQGVEK |
| | | DKFLINHASLTLSTLTVTSAHPEDSSFYICSASPRAGQLSSYNSPLH |
| | | FGNGTRLTVTEDLNKVFPPEVAVFEPSEAEISHTQKATLVCLATGF |
| | | FPDHVELSWWVNGKEVHSGVCTDPQPLKEQPALNDSRYCLSSRL |
| | | RVSATFWQNPRNHFRCQVQFYGLSENDEWTQDRAKPVTQIVSAE |
| | | AWGRADCGFTSVSYQQGVLSATILYEILLGKATLYAVLVSALVL |
| | | MAMVKRKDFGGSG |
| 1004 | RAS | MKKLLAMILWLQLDRLSGELKVEQNPLFLSMQEGKNYTIYCNYS |
| | | TTSDRLYWYRQDPGKSLESLFVLLSNGAVKQEGRLMASLDTKAR |
| | | LSTLHITAAVHDLSATYFCAVDKDGGYQKVTFGTGTKLQVIPNIQ |
| | | NPDPAVYQLRDSKSSDKSVCLFTDFDSQTNVSQSKDSDVYITDKC |
| | | VLDMRSMDFKSNSAVAWSNKSDFACANAFNNSIIPEDTFFPSPESS |
| | | CDVKLVEKSFETDTNLNFQNLSVIGFRILLLKVAGFNLLMTLRLW |
| | | SS |
| 1005 | RAS | CASSLYGGSISYEQYF |
| 1006 | RAS | CATDPGGFKTIF |
| 1007 | RAS | MGTRLLCWAALCLLGAELTEAGVAQSPRYKIIEKRQSVAFWCNPI |
| | | SGHATLYWYQQILGQGPKLLIQFQNNGVVDDSQLPKDRFSAERL |
| | | KGVDSTLKIQPAKLEDSAVYLCASSLYGGSISYEQYFGPGTRLTVT |
| | | EDLKNVFPPEVAVFEPSEAEISHTQKATLVCLATGFYPDHVELSW |
| | | WVNGKEVHSGVCTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDSR |
| | | GGGSG |
| 1008 | RAS | METLLGVSLVILWLQLARVNSQQGEEDPQALSIQEGENATMNCSY |
| | | KTSINNLQWYRQNSGRGLVHLILIRSNEREKHSGRLRVTLDTSKKS |
| | | SSLLITASRAADTASYFCATDPGGFKTIFGAGTRLFVKANIQNPDP |
| | | AVYQLRDSKSSDKSVCLFTDFDSQTNVSQSKDSDVYITDKCVLD |
| | | MRSMDFKSNSAVAWSNKSDFACANAFNNSIIPEDTFFPSPESSCDV |
| | | KLVEKSFETDTNLNFQNLSVIGFRILLLKVAGFNLLMTLRLWSS |
| 1009 | RAS | CAISESERYYEQYF |
| 1010 | RAS | CATFPNFGNEKLTF |
| 1011 | RAS | MGTRLFFYVALCLLWTGHMDAGITQSPRHKVTETGTPVTLRCHQ |
| | | TENHRYMYWYRQDPGHGLRLIHYSYGVKDTDKGEVSDGYSVSR |
| | | SKTEDFLLTLESATSSQTSVYFCAISESERYYEQYFGPGTRLTVTED |
| | | LKNVFPPEVAVFEPSEAEISHTQKATLVCLATGFYPDHVELSWWV |
| | | NGKEVHSGVCTDPQPLKEQPALNDSRYCLSSRLRVSATFWQNPR |
| | | NHFRCQVQFYGLSENDEWTQDRAKPVTQIVSAEAWGRADCGFTS |
| | | ESYQQGVLSATILYEILLGKATLYAVLVSALVLMAMVKRKDSRG |
| | | GSGG |
| 1012 | RAS | METLLGVSLVILWLQLARVNSQQGEEDPQALSIQEGENATMNCSY |
| | | KTSINNLQWYRQNSGRGLVHLILIRSNEREKHSGRLRVTLDTSKKS |
| | | SSLLITASRAADTASYFCATFPNFGNEKLTFGTGTRLTIIPNIQNPDP |
| | | AVYQLRDSKSSDKSVCLFTDFDSQTNVSQSKDSDVYITDKCVLD |
| | | MRSMDFKSNSAVAWSNKSDFACANAFNNSIIPEDTFFPSPESSCDV |
| | | KLVEKSFETDTNLNFQNLSVIGFRILLLKVAGFNLLMTLRLWSS |
| 1013 | RAS | SARDRGLVSLPSVEAFF |
| 1014 | RAS | ASYLSGSIYNEQFF |
| 1015 | RAS | ASSYSTERGTIY |
| 1016 | RAS | ASSLADIYEQY |
| 1017 | RAS | CASSARNDEAFF |
| 1018 | RAS | CASSLGDSEQYF |
| 1019 | RAS | CASSQRSNTGELFF |
| 1020 | RAS | CASGGRDSTDTQYF |
| 1021 | RAS | ASSTSFWEVNTEAF |
| 1022 | RAS | ASSKRGWPYEQY |
| 1023 | RAS | ASSLADIYEQY |
| 1024 | RAS | ASSSTDRIEAF |
| 1025 | RAS | ASTTFKTGRAIEKLF |
| 1026 | RAS | ASSSRGHSGTEAF |
| 1027 | RAS | ASSSRGHSGTEAF |
| 1028 | RAS | ATYKVGDEQF |
| 1029 | RAS | ASSDWLAGAKDEQY |
| 1030 | RAS | ASSLVASNEQF |
| 1031 | RAS | ASSLGLLLYNEQF |
| 1032 | RAS | ASSLGDSYEQYF |
| 1033 | RAS | ASSEWGSTGELF |
| 1034 | RAS | ASSEWGSTGELF |
| 1035 | RAS | ASSEYTMGTQY |
| 1036 | RAS | ASSLDFVLAGSYSYNEQ |
| 1037 | RAS | ASSQSGQGPYEQY |
| 1038 | RAS | ASSRTAMNTEAF |

SEQ ID NO: 1039: Human p53 amino acid sequence
MEEPQSDPSVEPPLSQETFSDLWKLLPENNVLSPLPSQAMDDLMLSPDDIEQWFTEDP
GPDEAPRMPEAAPPVAPAPAAPTPAAPAPAPSWPLSSSVPSQKTYQGSYGFRLGFLHS
GTAKSVTCTYSPALNKMFCQLAKTCPVQLWVDSTPPPGTRVRAMAIYKQSQHMTE
VVRRCPHHERCSDSDGLAPPQHLIRVEGNLRVEYLDDRNTFRHSVVVPYEPPEVGSD
CTTIHYNYMCNSSCMGGMNRRPILTIITLEDSSGNLLGRNSFEVRVCACPGRDRRTEE
ENLRKKGEPHHELPPGSTKRALPNNTSSSPQPKKKPLDGEYFTLQIRGRERFEMFREL
NEALELKDAQAGKEPGGSRAHSSHLKSKKGQSTSRHKKLMFKTEGPDSD
SEQ ID NO: 1040: Human PIK3CA amino acid sequence
MPPRPSSGELWGIHLMPPRILVECLLPNGMIVTLECLREATLITIKHELFKEARKYPLH
QLLQDESSYIFVSVTQEAEREEFFDETRRLCDLRLFQPFLKVIEPVGNREEKILNREIGF
AIGMPVCEFDMVKDPEVQDFRRNILNVCKEAVDLRDLNSPHSRAMYVYPPNVESSP
ELPKHIYNKLDKGQIIVVIWVIVSPNNDKQKYTLKINHDCVPEQVIAEAIRKKTRSML
LSSEQLKLCVLEYQGKYILKVCGCDEYFLEKYPLSQYKYIRSCIMLGRMPNLMLMA
KESLYSQLPMDCFTMPSYSRRISTATPYMNGETSTKSLWVINSALRIKILCATYVNVN
IRDIDKIYVRTGIYHGGEPLCDNVNTQRVPCSNPRWNEWLNYDIYIPDLPRAARLCLS
ICSVKGRKGAKEEHCPLAWGNINLFDYTDTLVSGKMALNLWPVPHGLEDLLNPIGV
TGSNPNKETPCLELEFDWFSSVVKFPDMSVIEEHANWSVSREAGFSYSHAGLSNRLA
RDNELRENDKEQLKAISTRDPLSEITEQEKDFLWSHRHYCVTIPEILPKLLLSVKWNS
RDEVAQMYCLVKDWPPIKPEQAMELLDCNYPDPMVRGFAVRCLEKYLTDDKLSQY
LIQLVQVLKYEQYLDNLLVRFLLKKALTNQRIGHFFFWHLKSEMHNKTVSQRFGLLL
ESYCRACGMYLKHLNRQVEAMEKLINLTDILKQEKKDETQKVQMKFLVEQMRRPD
FMDALQGFLSPLNPAHQLGNLRLEECRIMSSAKRPLWLNWENPDIMSELLFQNNEIIF
KNGDDLRQDMLTLQIIRIMENIWQNQGLDLRMLPYGCLSIGDCVGLIEVVRNSHTIM
QIQCKGGLKGALQFNSHTLHQWLKDKNKGEIYDAAIDLFTRSCAGYCVATFILGIGD
RHNSNIMVKDDGQLFHIDFGHFLDHKKKKFGYKRERVPFVLTQDFLIVISKGAQECT
KTREFERFQEMCYKAYLAIRQHANLFINLFSMMLGSGMPELQSFDDIAYIRKTLALD
KTEQEALEYFMKQMNDAHHGGWTTKMDWIFHTIKQHALN
SEQ ID NO: 1041: IL7R signaling domain
KKRIKPIVWPSLPDHKKTLEHLCKKPRKNLNVSFNPESFLDCQIHRVDDIQARDEVEG
FLQDTFPQQLEESEKQRLGGDVQSPNCPSEDVVITPESFGRDSSLTCLAGNVSACDAP
ILSSSRSLDCRESGKNGPHVYQDLLLSLGTTNSTLPPPFSLQSGILTLNPVAQGQPILTS
LGSNQEEAYVTMSSFYQNQ
SEQ ID NO: 1042: IL7R transmembrane domain
PILLTISILSFFSVALLVILACVLW
SEQ ID NO: 1043: CD80 extracellular domain
MGHTRRQGTSPSKCPYLNFFQLLVLAGLSHFCSGVIHVTKEVKEVATLSCGHNVSVE
ELAQTRIYWQKEKKMVLTMMSGDMNIWPEYKNRTIFDITNNLSIVILALRPSDEGTY
ECVVLKYEKDAFKREHLAEVTLSVKADFPTPSISDFEIPTSNIRRIICSTSGGFPEPHLS
WLENGEELNAINTTVSQDPETELYAVSSKLDFNMTTNHSFMCLIKYGHLRVNQTFN
WNTTKQEHFPDN
SEQ ID NO: 1044: CD58 extracellular domain
MVAGSDAGRALGVLSVVCLLHCFGFISCFSQQIYGVVYGNVTFHVPSNVPLKEVLW
KKQKDKVAELENSEFRAFSSFKNRVYLDTVSGSLTIYNLTSSDEDEYEMESPNITDTM
KFFLYVLESLPSPTLTCALTNGSIEVQCMIPEHYNSHRGLIMYSWDCPMEQCKRNSTS
IYFKMENDLPQKIQCTLSNPLFNTTSSIILTTCIPSSGHSRHR
SEQ ID NO: 1045: CD34 extracellular domain
MLVRRGARAGPRMPRGWTALCLLSLLPSGFMSLDNNGTATPELPTQGTFSNVSTNV
SYQETTTPSTLGSTSLHPVSQHGNEATTNITETTVKFTSTSVITSVYGNTNSSVQSQTS
VISTVFTTPANVSTPETTLKPSLSPGNVSDLSTTSTSLATSPTKPYTSSSPILSDIKAEIKC
SGIREVKLTQGICLEQNKTSSCAEFKKDRGEGLARVLCGEEQADADAGAQVCSLLLA
QSEVRPQCLLLVLANRTEISSKLQLMKKHQSDLKKLGILDFTEQDVASHQSYSQKT

### EXAMPLES

### Example 1

### Identification of KRAS G12V-Specific TCRs from the T Cell Repertoire of Healthy Donors

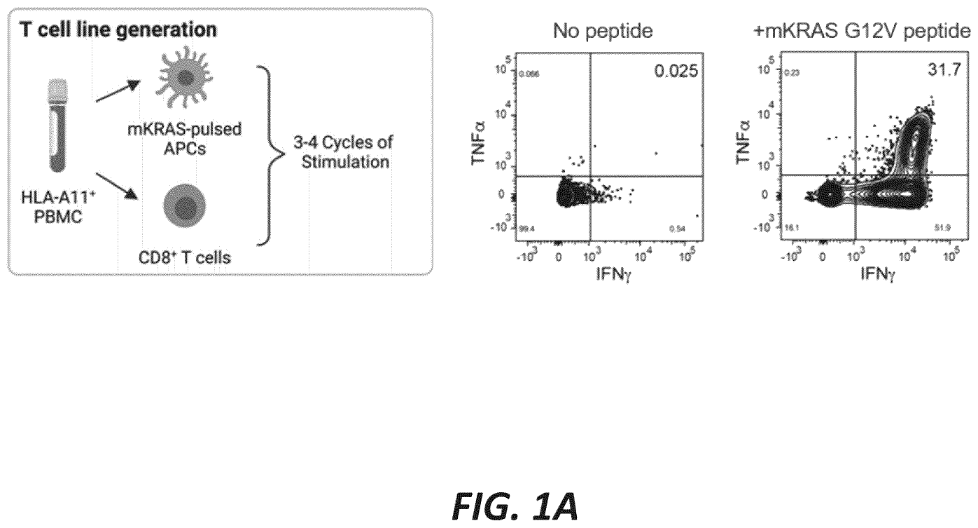

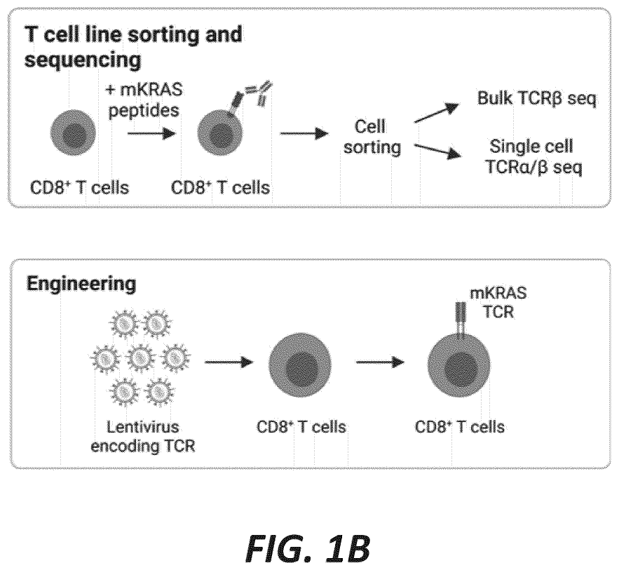

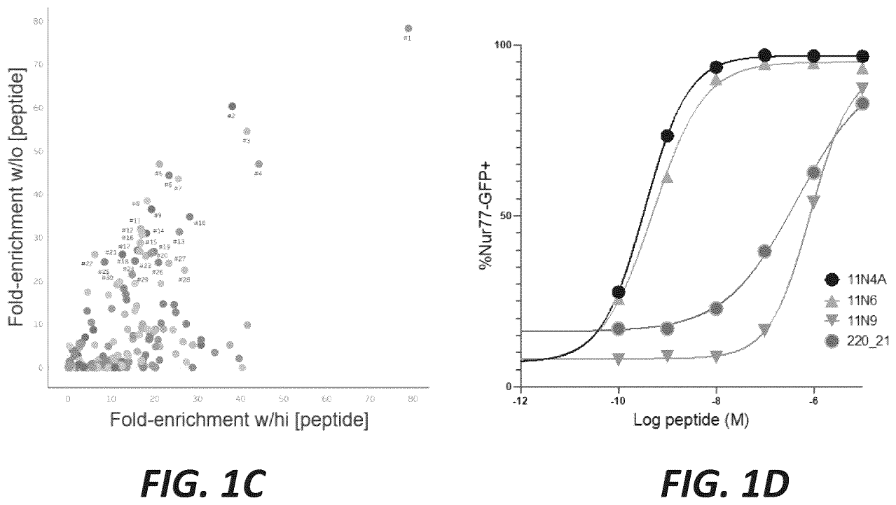

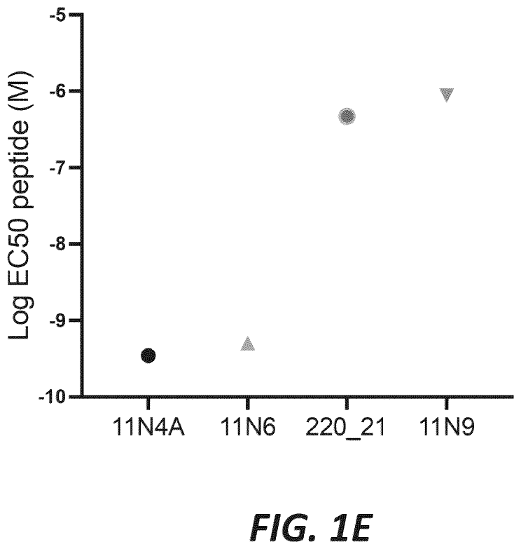

Dendritic cells derived from HLA-A11-positive healthy donor peripheral blood mononuclear cells (PBMCs) were generated, irradiated, and pulsed with KRAS-G12V₇₋₁₆ and KRAS-G12V₈₋₁₆ peptides. These were incubated for 8-10 days with autologous CD8⁺ T cells to induce activation/expansion of antigen-specific CD8+ T cells. These polyclonal T cell lines were then restimulated and expanded for 8-10 days two times with peptide-pulsed irradiated autologous PBMCs to further expand antigen specific clones. This process was conducted across ten lines of CD8+ T cells from each of 15 HLA-matched donors. (Ho W Y et al., J Immunol Methods. 2006; 310(1):40-52. doi:10.1016/j.jim.2005.11.023) (FIG. 1A).

To identify TCRs with strong binding to their cognate peptide (i.e., a KRAS peptide) presented in the context of HLA-A11, T cells were stimulated overnight with titrated concentrations of cognate KRAS G12V peptides and CD137 upregulation was assessed by flow cytometry. Cells expressing CD137 were isolated by flow cytometric cell sorting and TCR beta repertoire analysis was performed (Adaptive Biotechnologies, Seattle, WA). TCR clonotypes that were highly enriched in CD137+ populations and that responded to low concentrations of peptide were identified, and TCR alpha/beta pairing was determined by 10× single cell RNAseq analysis on similarly sorted populations (10× Genomics, Pleasanton, CA). A representative analysis of clonotype enrichment in CD137+ sorted populations compared to total unsorted cells treated with low and high peptide concentrations is shown in FIG. 1B. Paired TCRalpha/beta sequences from identified clonotypes were assembled and synthesized as P2A-linked expression cassettes and lentivirally transduced into reporter Jurkat cells that express GFP under the control of the Nur77 locus (Nur77-GFP-Jurkats). Peptide dose-dependent responses for each TCR were assessed by analyzing GFP expression following overnight culture with A11 target cells pulsed with decreasing concentrations of peptide as indicated (FIG. 1C). Dose-response curves were fitted by non-linear regression, and EC50 values were calculated using Graphpad Prism (Boston, MA) (FIGS. 1D, 1E).

### Example 2

### Functional Avidity of KRAS-G12V-Specific TCRs Expressed in Primary CD8⁺ T Cells

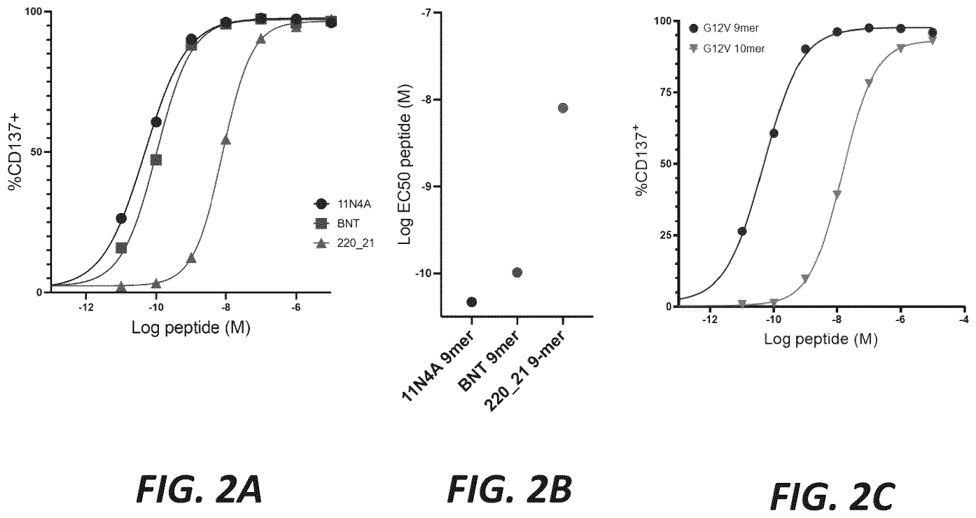

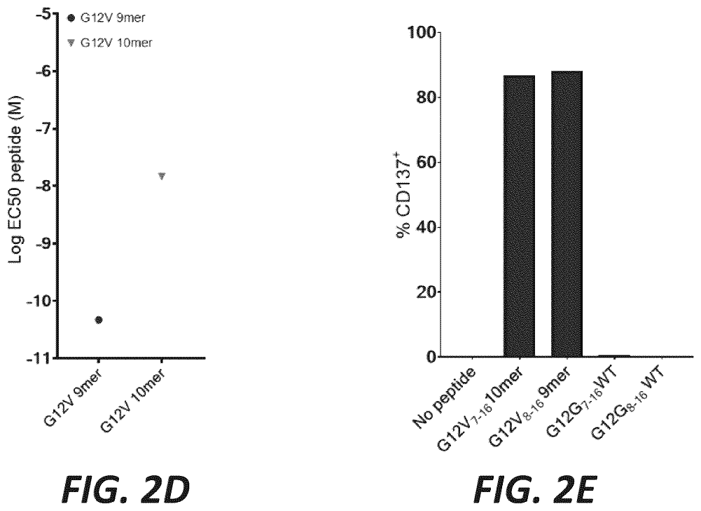

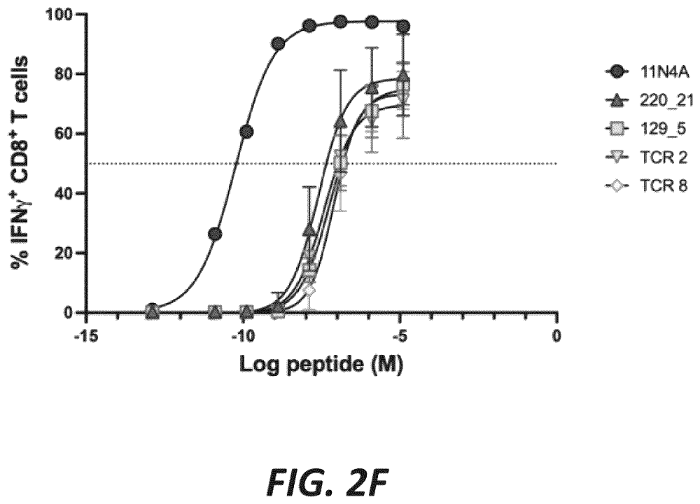

Primary CD8⁺ T cells were transduced with polynucleotides encoding KRAS-G12V-specific TCRs, sort purified, and expanded. Sort-purified T cells were then cultured overnight with decreasing concentrations of KRAS-G12V₈₋₁₆ peptide and CD137 expression was assessed by flow cytometry. Dose-response curves were fitted by non-linear regression, and EC50 values were calculated using Graphpad Prism (FIGS. 2A, 2B). In this experiment, TCR 11N4A was compared to a KRAS G12V-specific TCR “220_21” (see SEQ ID NOs:61 and 62 herein), and to TCR “BNT”, having variable domains encoded by SEQ ID NOs:54 (Vα) and 57 (Vβ) of US Publication No. US 2021/0340215A1 (see also SEQ ID NOs:59 and 60 herein). All TCRs were encoded by lentivirus in TCRβ-P2A-TCRα expression cassettes.

TCR 11N4A was compared to 220_21 and other TCRs using a similar assay, measuring peptide antigen dose-response for IFN-γ expression (FIG. 2C).

### Example 3

### KRAS-G12V-Specific TCR-Transduced T Cell Recognition of KRAS-G12V Expressing Tumor Cell Lines

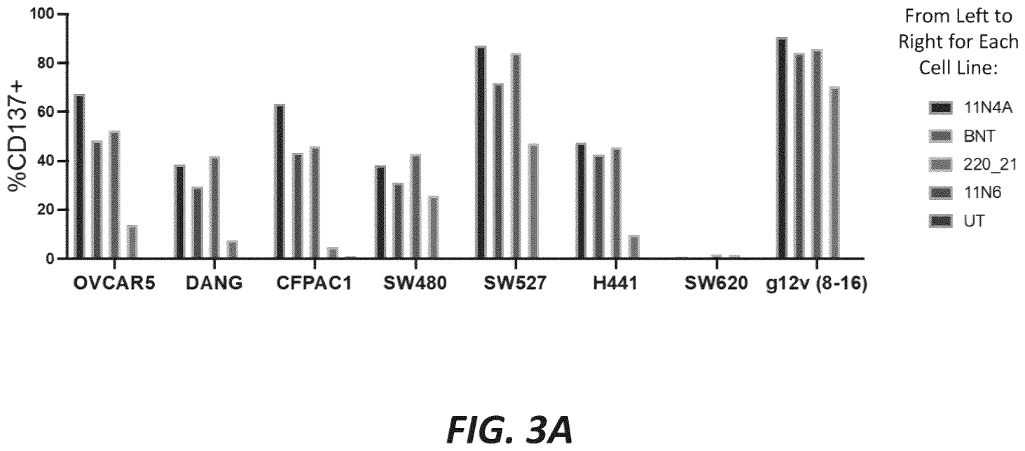

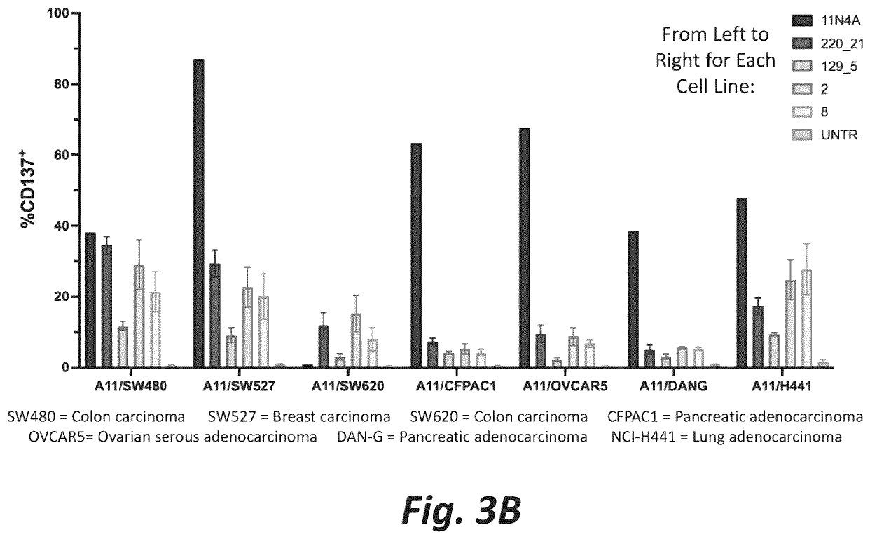

Primary CD8+ T cells were transduced with KRAS-G12V-specific TCRs, sort purified, and expanded. Sort-purified T cells were cultured overnight with tumor cell lines that express mutant KRAS-G12V. T cells cultured with 1 mg/ml of KRAS-G12V₈₋₁₆ peptide were included as a positive control. T cell responses were assessed by measuring CD137 expression in response to TCR signaling (FIGS. 3A-3B). Tumor lines were first transduced to express HLA-A11 as-needed and sort-purified for HLA-A11 expression.

### Example 4

### Specific Killing of KRAS-G12V-Expressing Tumor Cell Lines by CD8⁺ T Cells Expressing KRAS-G12V-Specific TCRS

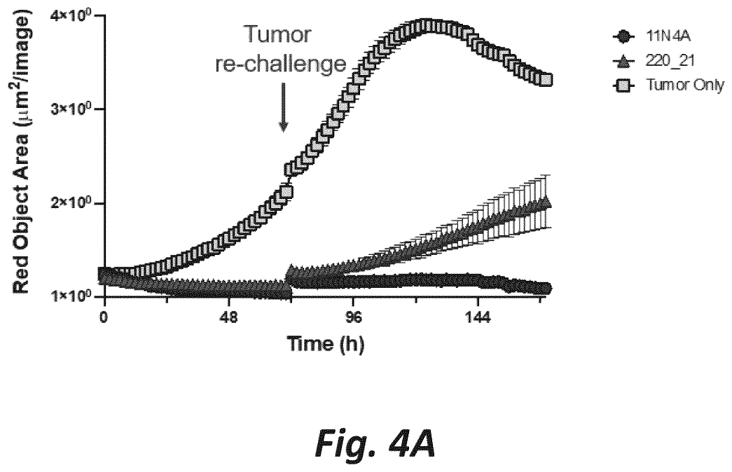

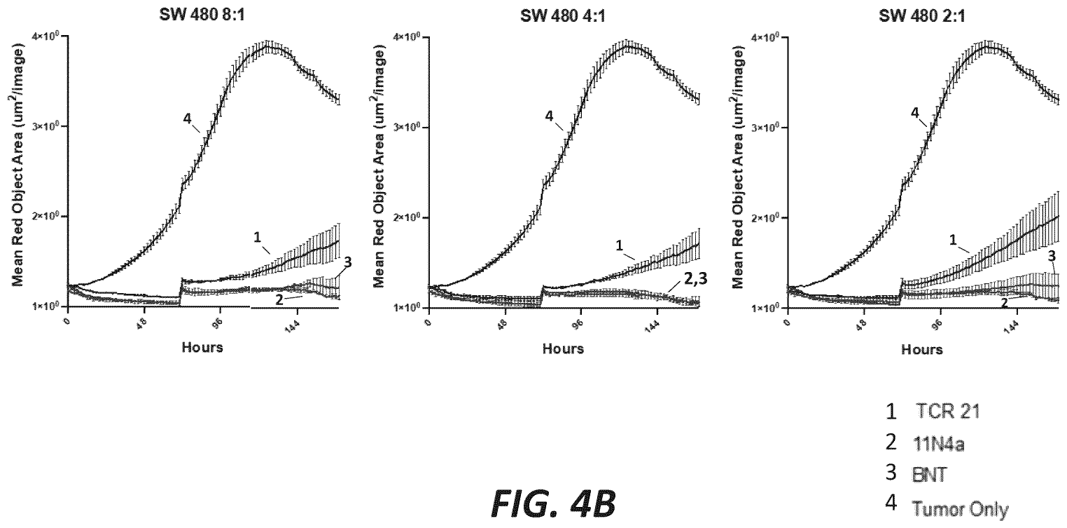

Red fluorescent SW480 cells, a KRAS-G12V expressing tumor cell line transduced to express HLA-A11, were cocultured with TCR-transduced T cells as indicated and enumerated over time by live cell imaging using the IncuCyte S3 microscope and software package. CD8⁺ T cell cytotoxicity is indicated by a decrease in the total red target cell area per well as compared to no treatment wells. Additional tumor cells were added at 72 hours to assess TCR-mediated tumor cell lysis by transduced T cells in the presence of persistent antigen. (FIG. 4A). In a separate experiment, three increasingly stringent effector:target cell ratios were used to measure relative TCR-mediated tumor lysis in conditions when T cells are limiting. Data are shown in FIG. 4B.

### Example 5

### Mutational Scan to Characterize

### The Peptide Binding Motif of TCR 11N4A

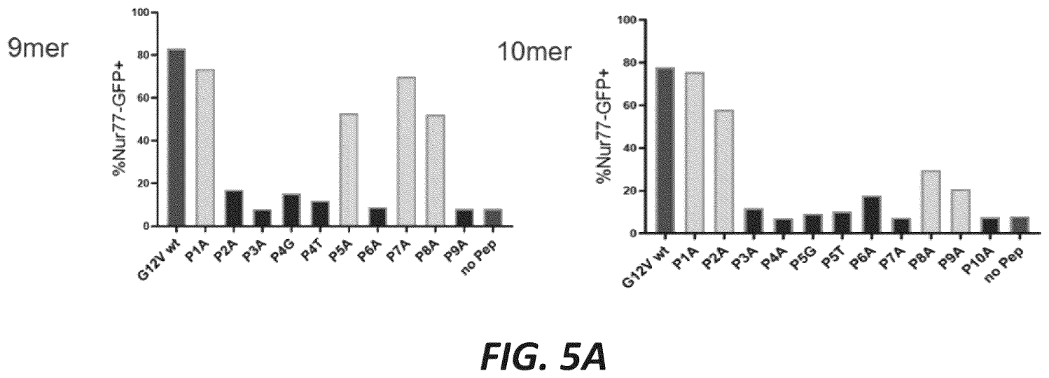

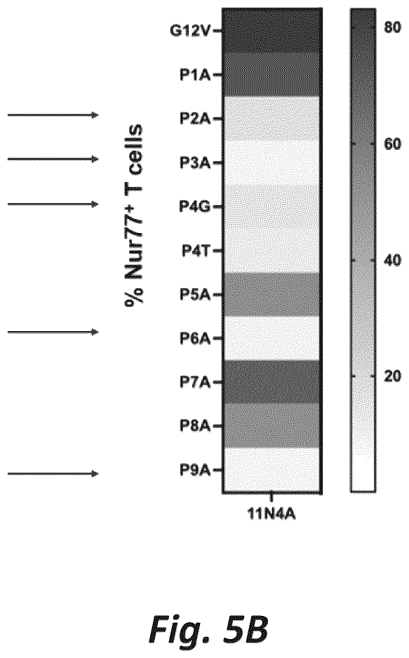

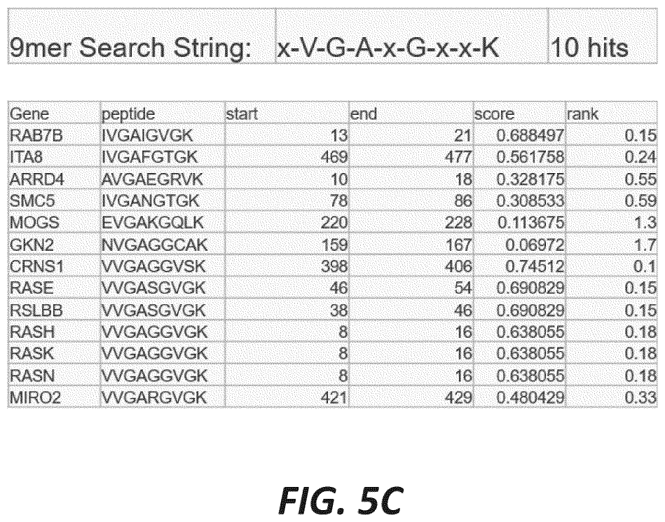

To assess the potential cross-reactivity of TCR 11N4A, a mutational scan was performed to identify peptide residues critical for TCR binding. Peptides were synthesized in which each residue of the cognate KRAS-G12V peptide was changed to an alanine. Position 4 of the cognate 9mer peptide (position 5 of the 10mer peptide) already contains an alanine, so peptides were generated that contain a glycine or a threonine at this position. TCR 11N4A-transduced Nur77-GFP-Jurkats were cultured overnight with HLA-A11⁺ B-LCL cells pulsed with 1 mg/ml of each peptide followed by flow cytometric analysis of GFP expression. Peptides that contained a substitution at position 1, 5, 7 or 8 of the 9mer and the corresponding positions of the 10mer were able to elicit a response from cells expressing TCR 11N4A, indicating that TCR 11N4A can recognize peptides with other amino acids at these positions (FIGS. 5A and 5B). A search of the human proteome for similar motifs was performed using ScanProsite (prosite.expasy.org/scanprosite/) using the search string: x-V-G-A-x-G-x-x-K (SEQ ID NO:4). The resulting potentially cross-reactive peptides are shown in FIG. 5C with predicted HLA-A11 binding data from IEDB (NetPanMHC4.1) shown as percentile rank (lower is better) and score (higher is better). These data include two peptides that each appear in multiple proteins (RASE and RSLBB; wildtype RAS proteins RASH, RASK and RASN).

### Example 6

### Analysis of TCR 11N4A Reactivity to Potentially Cross-Reactive Peptides

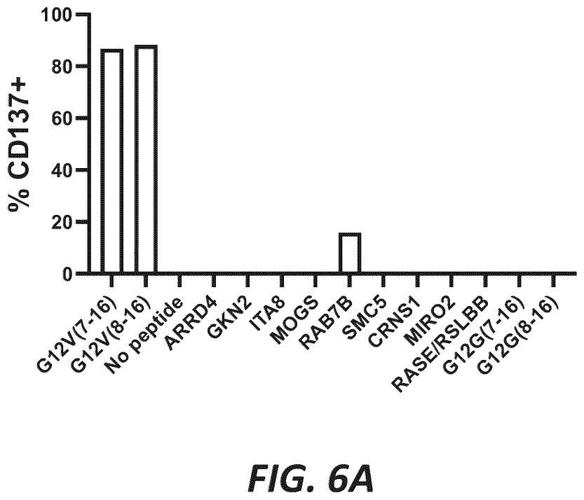

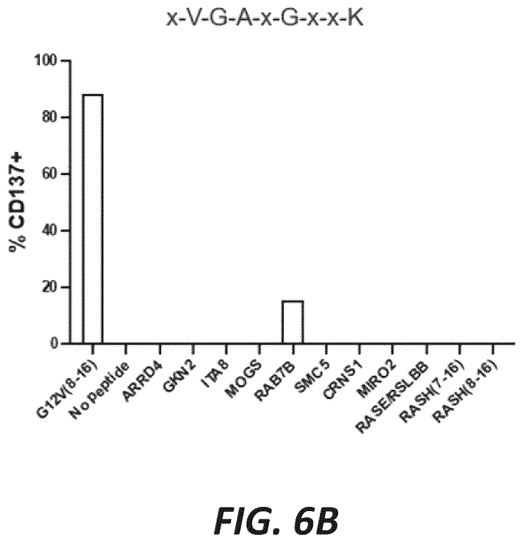

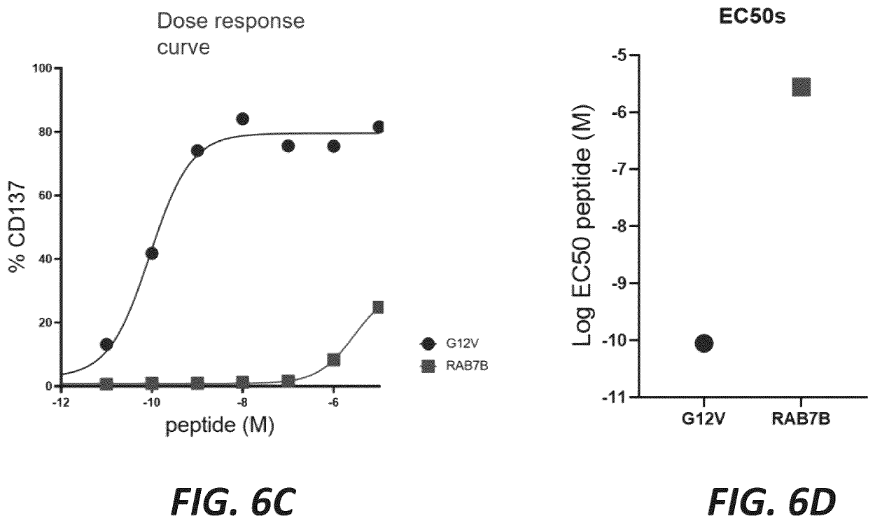

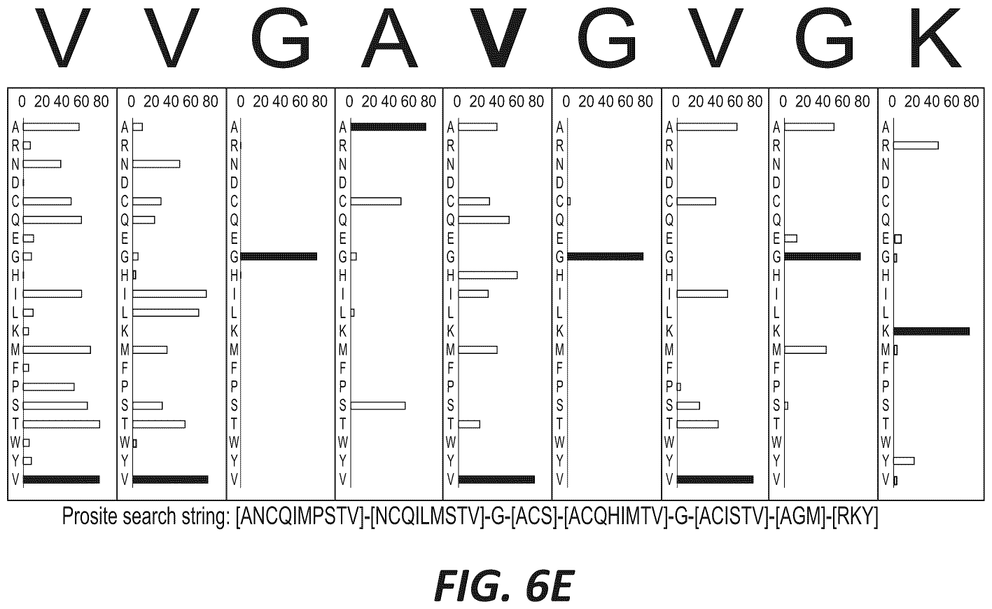

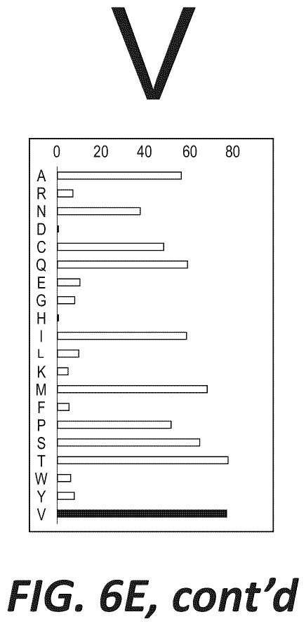

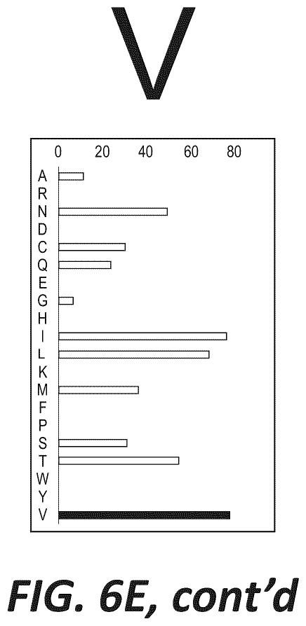

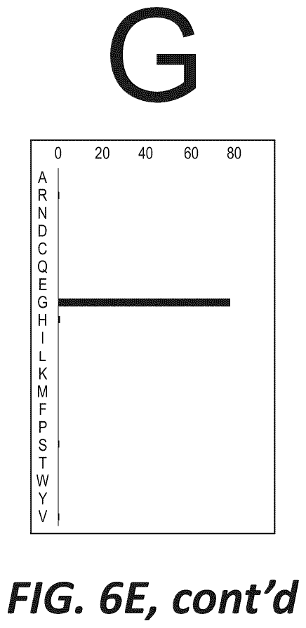

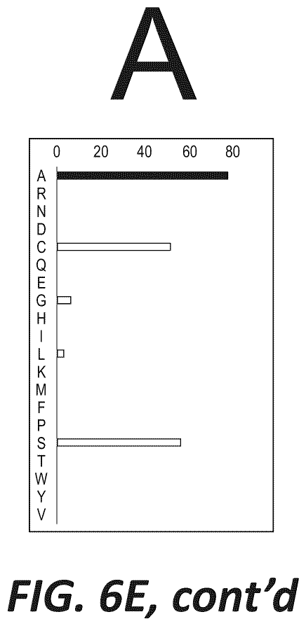

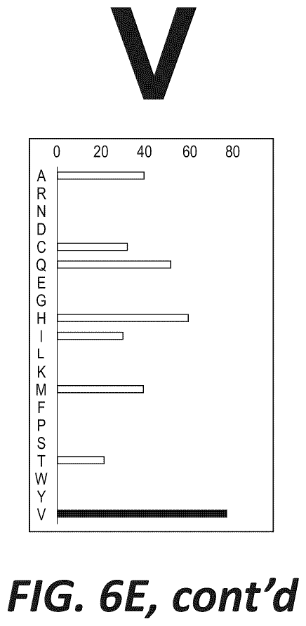

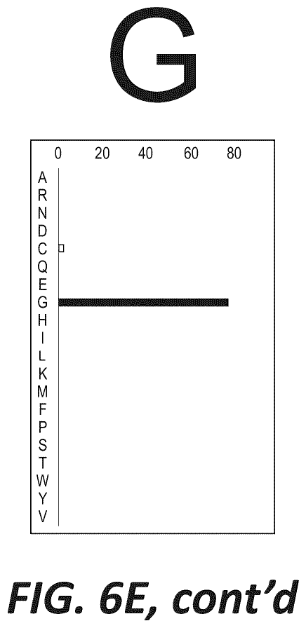

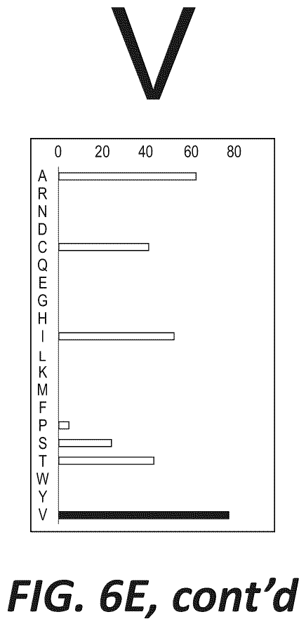

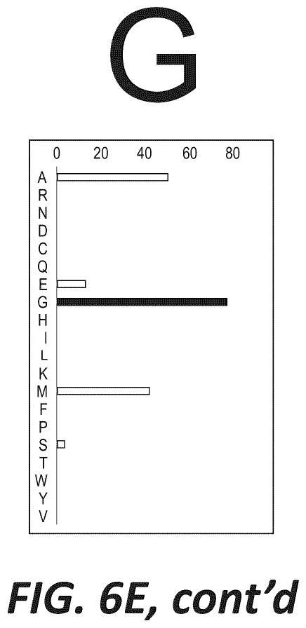

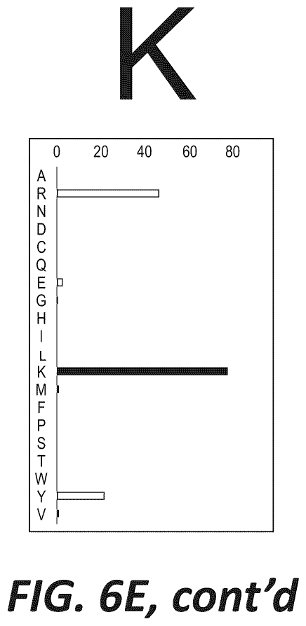

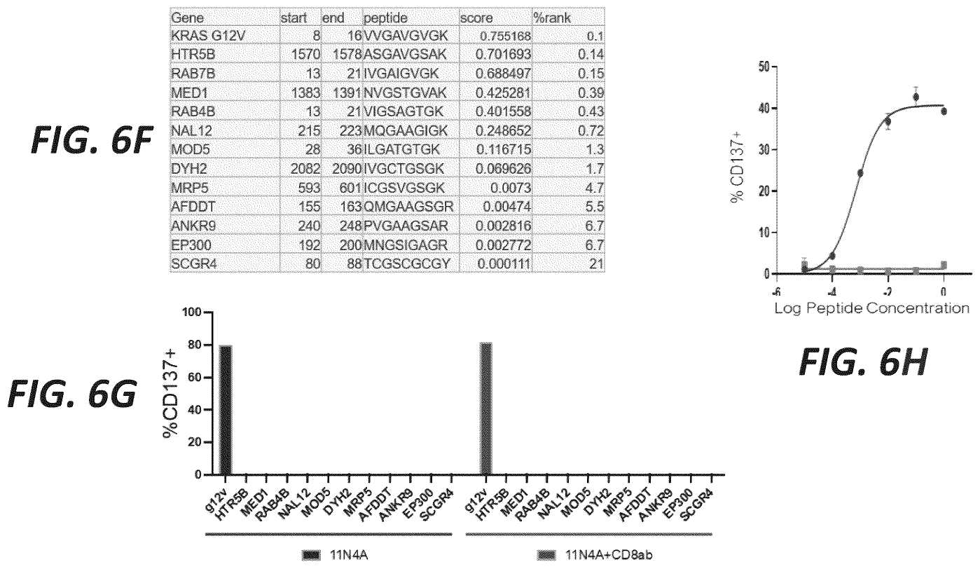

TCR 11N4A-transduced donor-derived CD8⁺ T cells were cultured overnight with each of the identified potential cross-reactive peptides or cognate KRAS-G12V peptides (1 mg/ml), and activation-induced CD137 expression was assessed by flow cytometry. No response was detected from any peptides, except for a low-level response (<20%) from a RAB7B-derived peptide (FIGS. 6A, 6B). To further assess functional avidity of TCR 11N4A against the RAB7B peptide, sort-purified TCR 11N4A-transduced T cells were cultured overnight with decreasing concentrations of KRAS-G12V₈₋₁₆ peptide or RAB7B peptide and CD137 expression was assessed by flow cytometry. Dose-response curves were fitted by non-linear regression (FIGS. 6C and 6H), and EC50 values were calculated using Graphpad Prism (FIG. 6D).

The calculated EC50 for RAB7B peptide was ˜35 mg/ml, a very high concentration of peptide that can result in a density of peptide-loaded MHC on the target cell surface that is several orders of magnitude greater than the density of any particular peptide/HLA-A11 complex presented on the surface of a typical cell. Cells normally present a diverse array of processed cellular proteins, at a density that has been reported to be in the range of 10-150 peptide/MHC complexes per cell for several well-presented self-peptides (Bossi et al., Oncoimmunology. 2013; 2(11):e26840; Liddy et al., Nat Med. 2012; 18(6):980-7; Purbhoo et al., J Immunol. 2006; 176(12):7308-16.) To specifically characterize the relationship between peptide concentration and epitope presentation by T2 cells, soluble, high-affinity TCRs coupled with single-molecule fluorescence microscopy were used to quantify several well-characterized self-peptides on peptide-pulsed T2 cells. (Bossi et al.). The results of this analysis suggest that peptide concentrations in the low nanomolar range (1-10 nM) are required to approximate physiological levels of presented antigen.

In contrast, even at the high dose of 10 mg/ml (˜10 mM), only a low-level response by TCR 11N4A-transduced T cells was observed (˜25% of T cells responding, compared to >80% of T cells responding to the cognate KRAS-G12V peptide). Importantly, no response by TCR 11N4A-transduced T cells was observed with peptide concentrations of 100 nM or lower. These data support that TCR 11N4A-transduced T cells do not have sufficient affinity for the RAB7B peptide to recognize the naturally processed and presented epitope.

To further assess the potential for TCR cross-reactivity, CD8⁺ T cells expressing TCR 11N4A were cultured overnight with a comprehensive panel of positional scanning peptides containing a substitution of every possible amino acid at each position of the cognate KRAS G12V peptide (a library of 172 peptides was synthesized to 90% purity spanning all possible amino acid substitutions of the reference peptide (VVGAVGVGK)). Whereas alanine scanning mutagenesis assesses serial substitutions of alanine at each of the peptide positions, XScan evaluates all other 19 amino acids at each position of the target KRAS^{G12V} peptide (Border et al. (2019) Oncoimmunology, 8(2): e1532759; doi.org/10.1080/2162402X.2018.1532759). The percentage of T cells expressing CD137 in response to each peptide is shown in FIG. 6E, organized by peptide position.

From these data, a potentially cross-reactive peptide motif was determined, and peptides that match that motif were identified by searching the human proteome using ScanProsite (prosite.expasy.org/scanprosite/). Peptides that elicited a response of greater than 15% were considered positive in this assay. The potentially cross-reactive peptides identified from the ScanProsite search are shown in the table (FIG. 6F). RAB7B, the only peptide identified as cross-reactive in the mutational scan analysis, was the only peptide that was also identified in the Xscan analysis, validating the utility of this type of analysis. The additional peptides identified were synthesized and added at 100 ng/ml to sort-purified primary CD8⁺ T cells transduced to express TCR 11N4A or TCR 11N4A+CD8αβ co-receptor (e.g. exogenous CD8αβ co-receptor). After overnight culture, activation-induced CD137 expression was assessed by flow cytometry. No reactivity was detectable for any of the additional identified peptides (FIG. 6G).

### Example 7

### Alloreactivity Screen for TCR 11N4A with or without CD8αβ Shows No Alloreactivity Against B-LCLs Expressing Common HLA Alleles

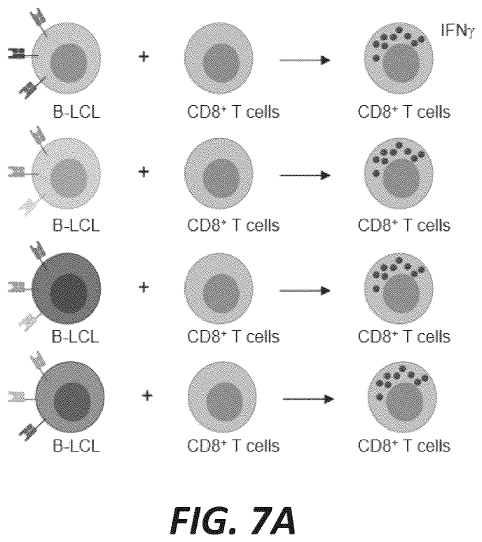

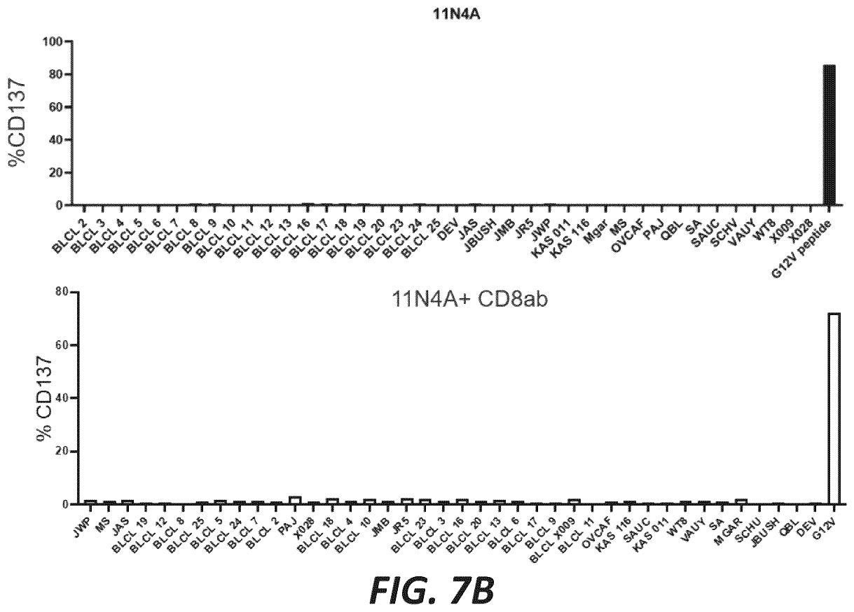

To determine whether TCR 11N4A exhibits alloreactivity towards common non-A11 HLA alleles, sort purified primary CD8+ T cells were transduced with either a polynucleotide encoding TCR 11N4A alone, or an alternative construct that contains CD8 alpha and CD8 beta coding sequences in addition to the TCR 11N4A alpha and beta chains and cultured overnight with a panel of B-LCL cell lines that express a diverse set of HLA alleles that are commonly found in the US population (FIG. 7A). Activation-induced CD137 expression after overnight culture was assessed by flow cytometry (FIG. 7B).

### Example 8

### Specific Killing Activity of CD4+ T Cells Expressing TCR 11N4A and a CD8 Co-Receptor

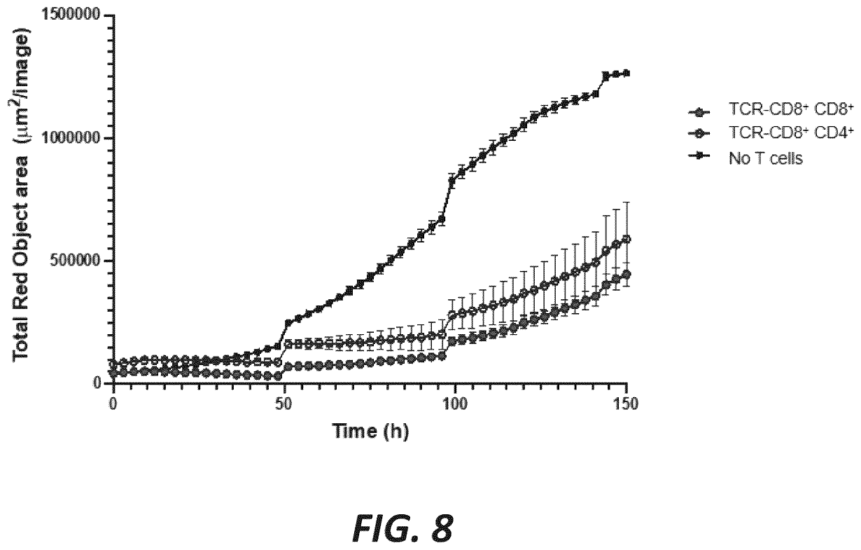

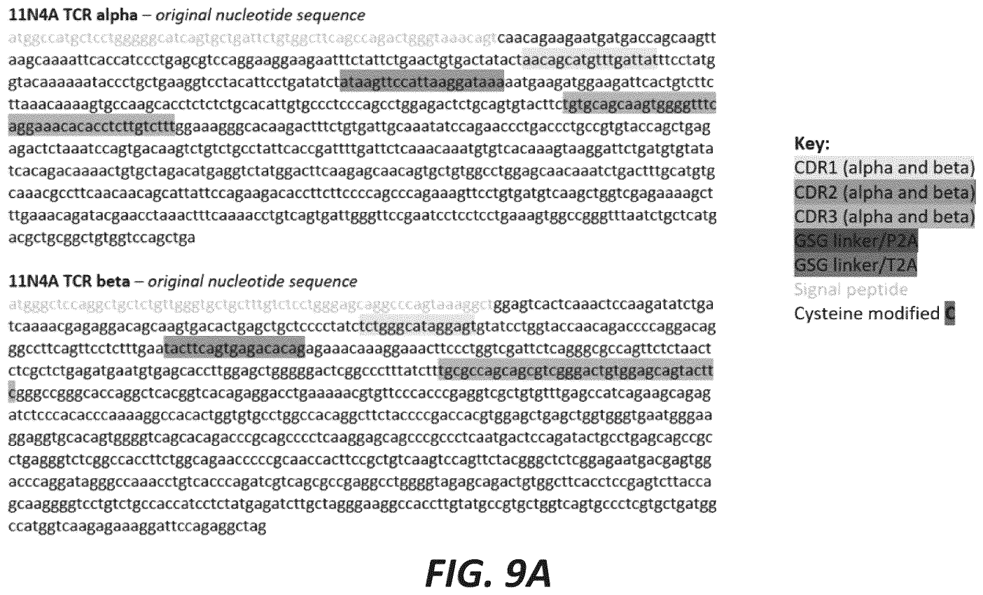

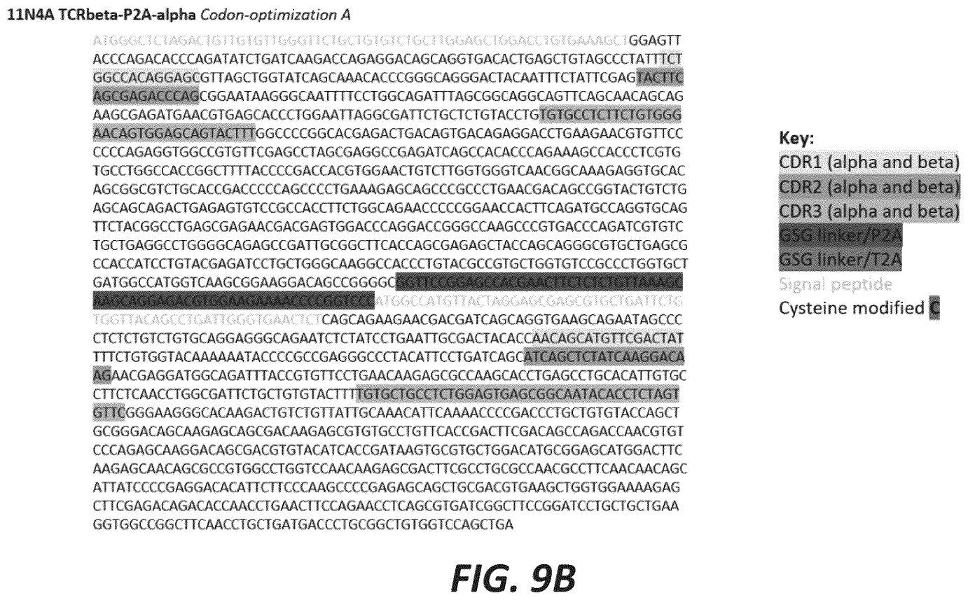

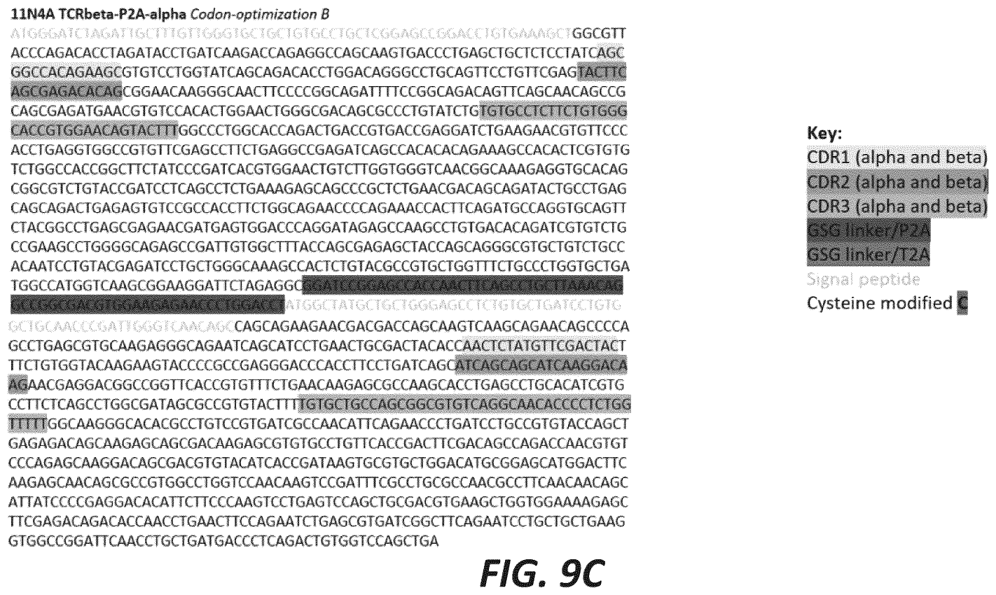

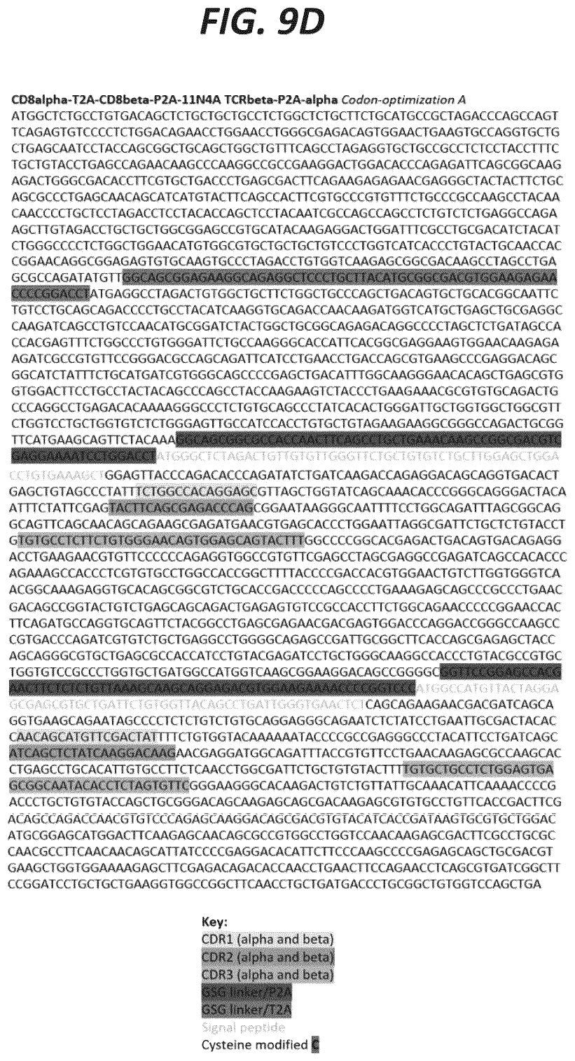

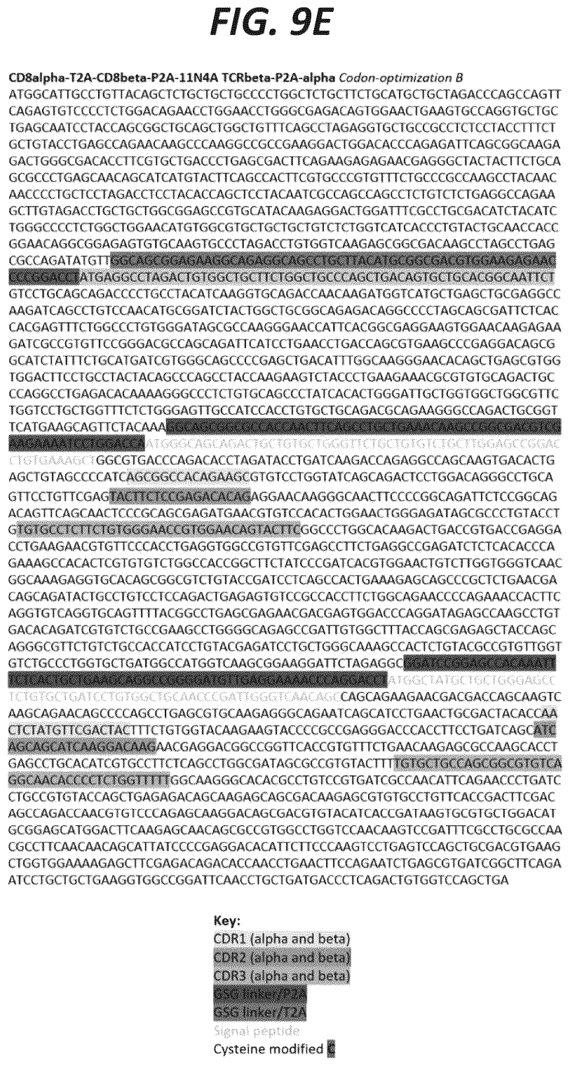

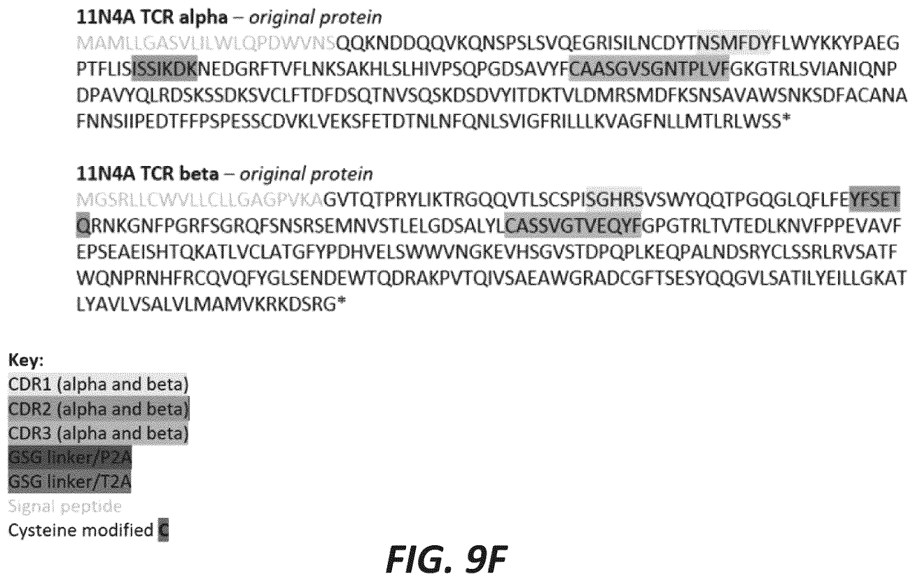

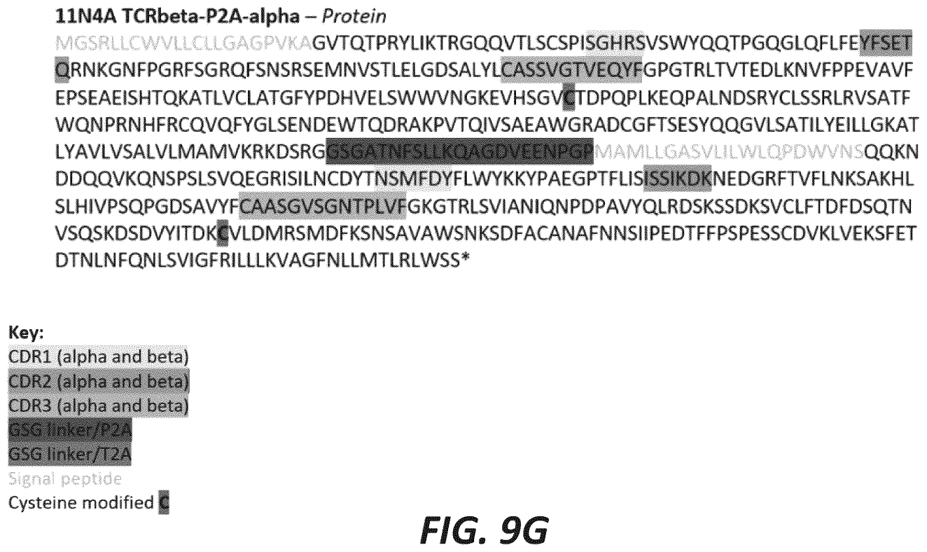

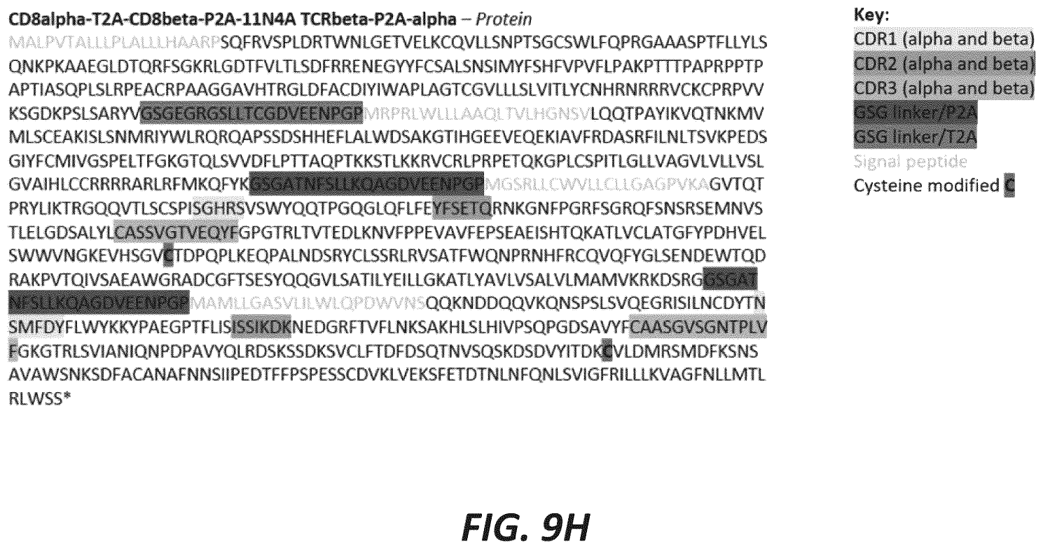

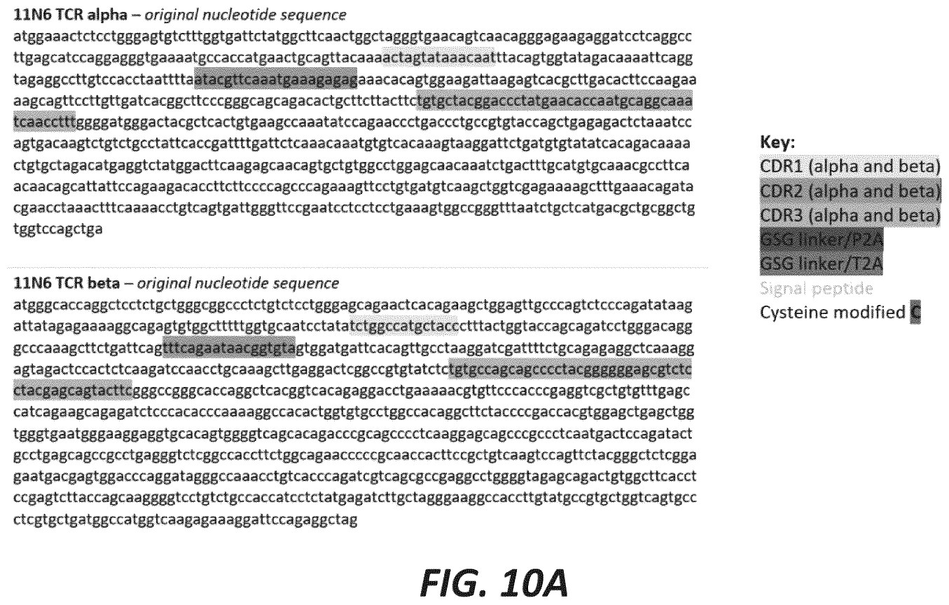

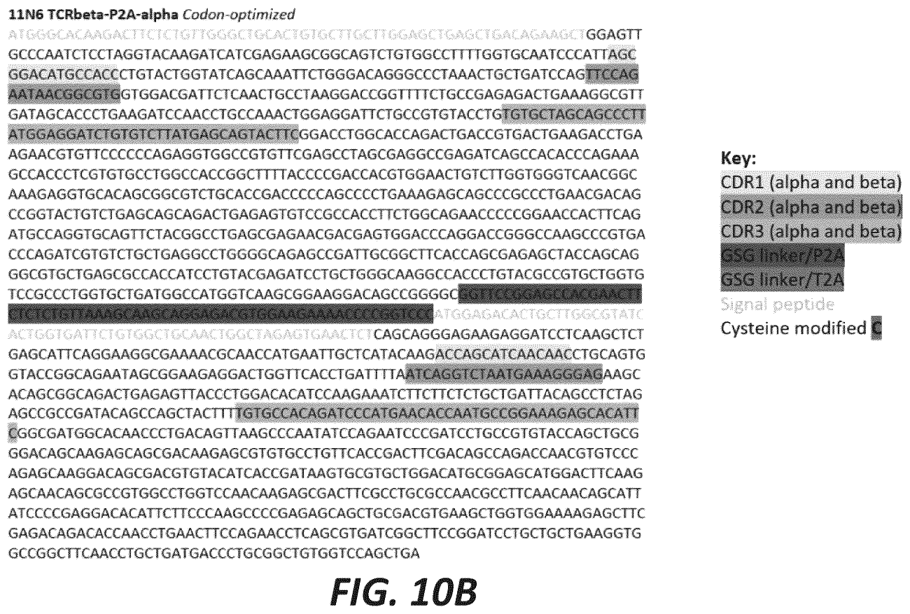

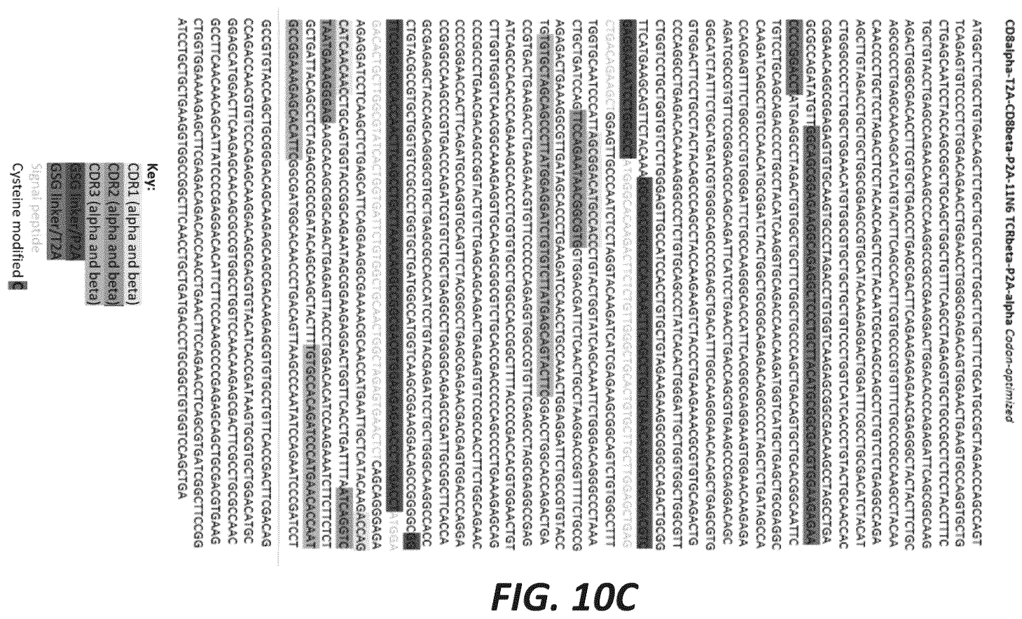

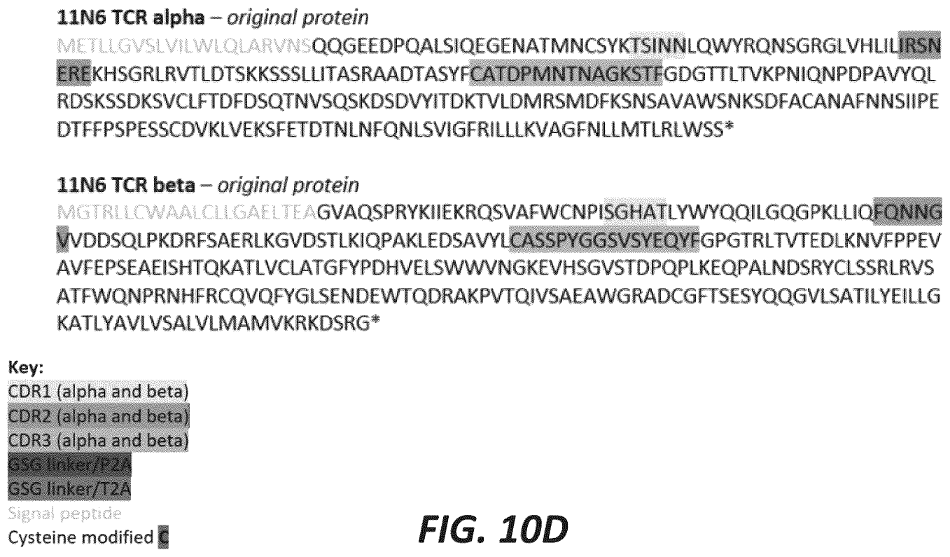

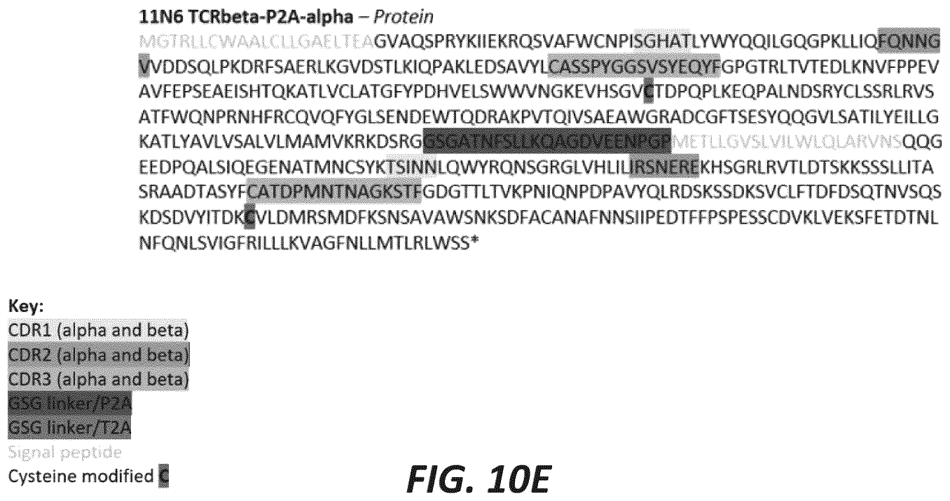

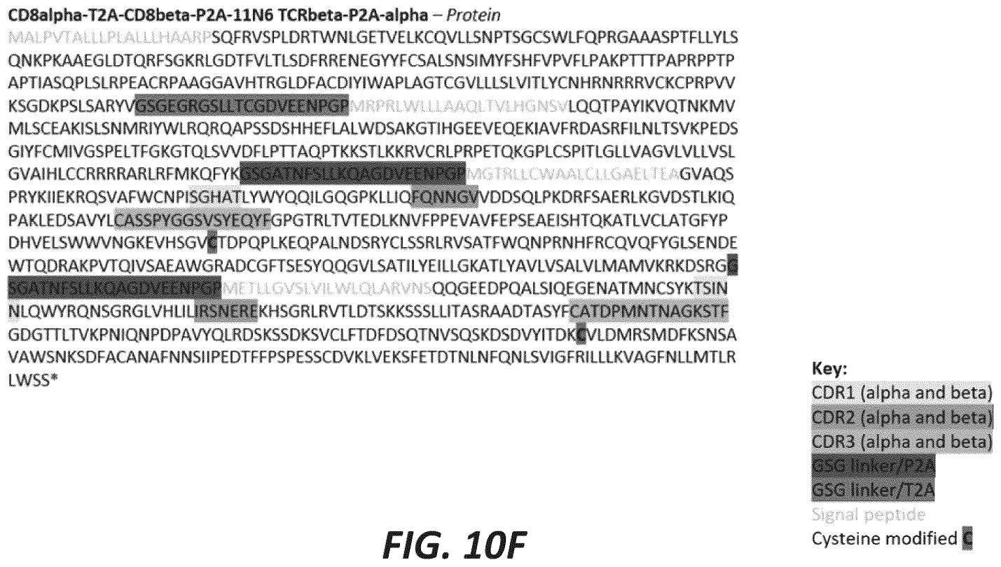

CD4+ and CD8+ T cells were transduced to express TCR 11N4A and a CD8αβ co-receptor (e.g. exogenous CD8αβ co-receptor). Killing activity of the engineered T cells was assessed using an IncuCyte assay (FIG. 8).

### Example 9

### Enhancing T Cell Survival and Function with Addition of FAS/41BB Fusion Proteins to T Cells Expressing TCRs Targeting KRAS

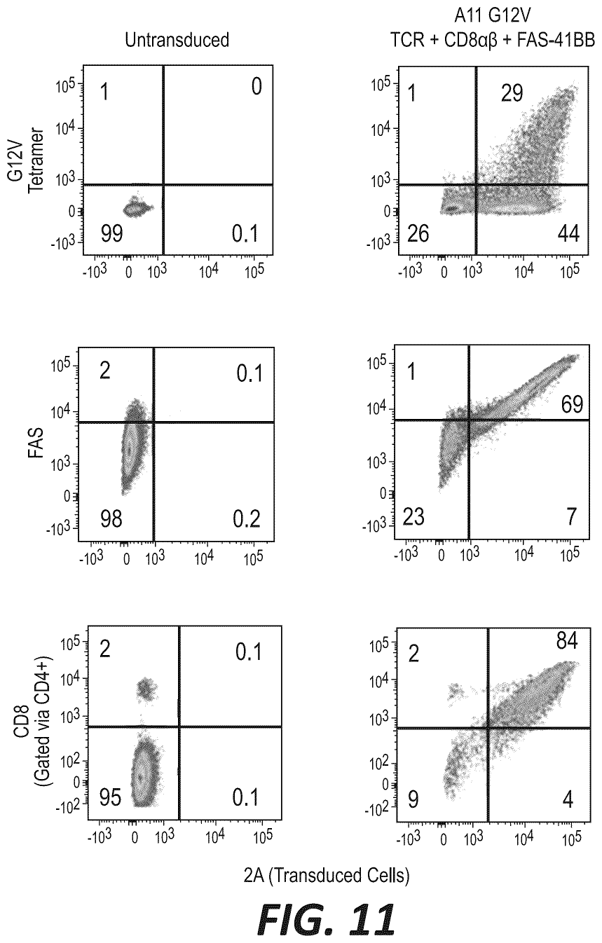

Host cells described herein also include host cells comprising fusion proteins comprised of the extracellular domain of Fas, or portions thereof, and an intracellular signaling domain of 41BB. The extracellular component may comprise all or a portion of the extracellular domain of Fas. In some embodiments, the transmembrane component may be comprised of the domain of Fas, 41BB, or CD28, or portions thereof. The extracellular component may comprise all or a portion of the extracellular domain of Fas or may be truncated to preserve maintain a short spatial distance between the cells (-9aas) upon receptor-ligand interaction. In some other example Fas-41BB fusion proteins, the transmembrane component comprises the transmembrane domain of 41BB. Additionally, a Fas-41BB construct has the capacity to convert a signal initiated by the binding of Fas to its target into a positive (e.g., costimulatory) signal generated by the 41BB intracellular signaling domain. FIG. 11 (FIG. 11) illustrates some of the potential advantages of including Fas-41BB fusion proteins alongside TCRs according to the current disclosure.

Fas-41BB fusion proteins and a transgenic TCR (e.g., TCR 11N4A) can be co-expressed in transduced murine T cells. Accordingly, cells comprising such a fusion protein (e.g., the nucleotide sequence of SEQ ID NO: 83 or the protein sequence of SEQ ID NO: 80) and the TCR 11N4A were generated using the general methods described herein.

FIG. 11 demonstrates that cells transduced with a lentiviral construct bearing TCR 11N4A, CD8αβ co-receptor (e.g. exogenous CD8αβ co-receptor), and FAS/41BB fusion protein successfully express all three markers. Shown is representative flow cytometric plots of engineered TCR expression (G12V Tetramer, top), FAS-41BB fusion protein (FAS, middle), and exogenous CD8 (CD8 gated via CD4+, bottom) in primary human CD4/CD8 T cells either untransduced (left) or engineered to express A11 G12V TCR+CD8αβ+FAS41BB (right). Intracellular 2A staining (x-axis) identified transduced cells via 2A elements that separate the individual parameters within the lentiviral construct. CD8 analysis included only CD4+ T cells, thus excluding endogenous CD8+ T cells. T cells activated with anti-CD3/CD28 beads for 2 days, lentivirally transduced, and analyzed by flow cytometry after 3 days of expansion.

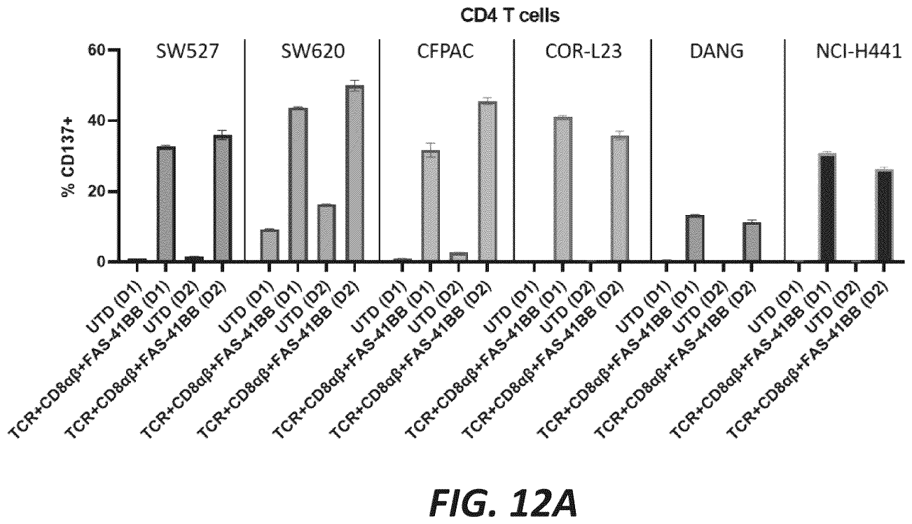

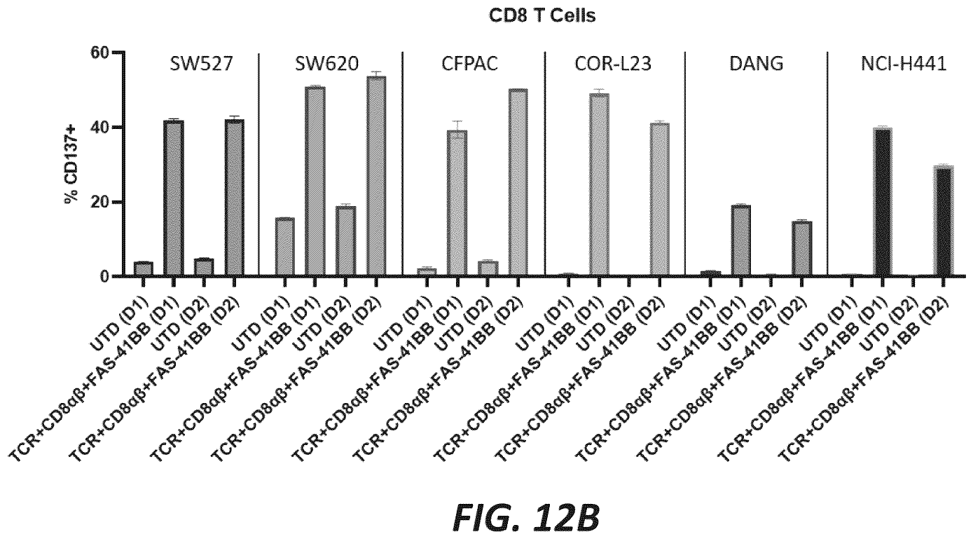

To confirm that T cells transduced with TCR 11N4A, CD8αβ co-receptor (e.g. exogenous CD8αβ co-receptor), and a FAS/41BB fusion protein are able to respond to endogenously expressed and presented KRAS^{G12V}, a panel of tumor cell lines derived from diverse indications and expressing HLA-A*11:01 and KRAS^{G12V} antigen was tested (FIG. 12). Research-grade products derived from 2 different donors were activated by co-culture with all KRAS^{G12V}-expressing tumor cell lines tested, whereas untransduced T cells (UTD) from the same donors exhibited minimal activation as assessed by CD137 FACS staining. CD4+ and CD8+ T cells are activated at similar levels by the tumor cell panel demonstrating the ability of CD8α/β coreceptor to enable MHC class I restricted responses in CD4+ T cells (FIGS. 12A, 12B)..

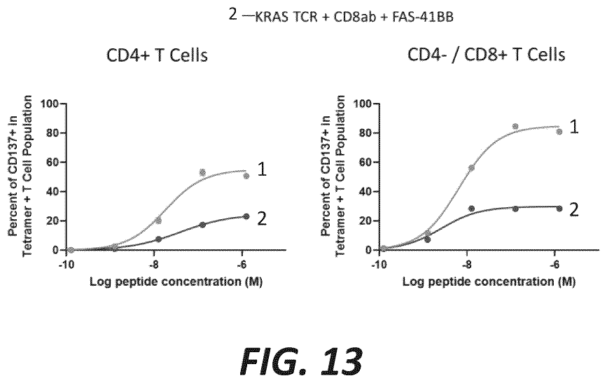

As shown in FIG. 13 (FIG. 13), a FAS-41BB fusion protein improved KRAS engineered T cell sensitivity of re-stimulated T cells. In this experiment, T cells comprising the TCR 11N4A against KRAS, CD8αβ co-receptor (e.g. exogenous CD8αβ co-receptor), and a FAS/41BB fusion protein according to SEQ ID NO: 80 (alongside the indicated controls) were treated with escalating G12V peptide concentration to stimulate the T cell, and the percentage of cells stimulated to express the CD137 receptor was assessed. Inclusion of the FAS-41BB fusion protein effectively increased the magnitude of the stimulatory response to the G12V peptide.

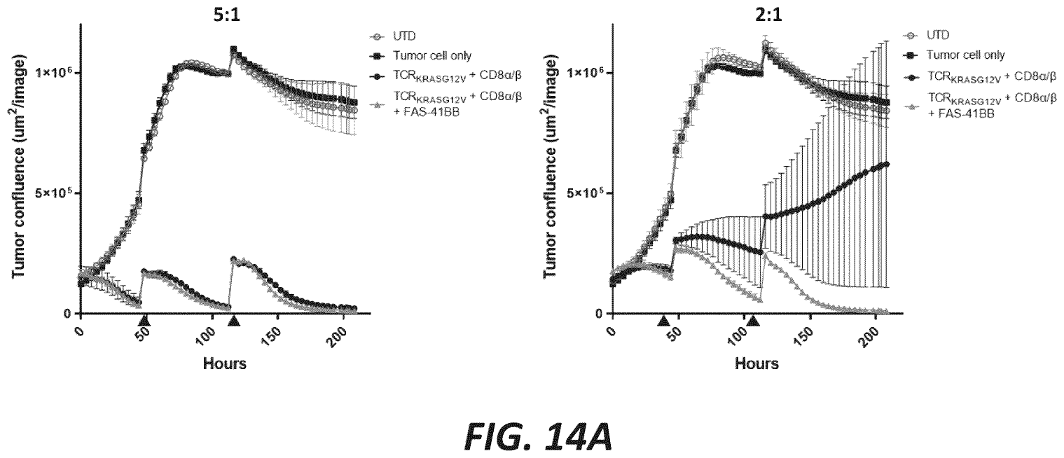

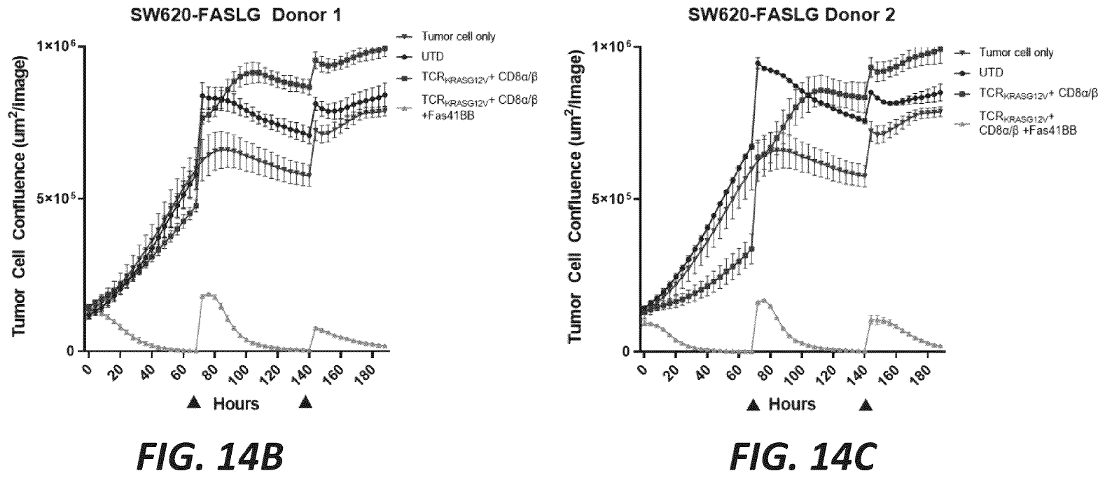

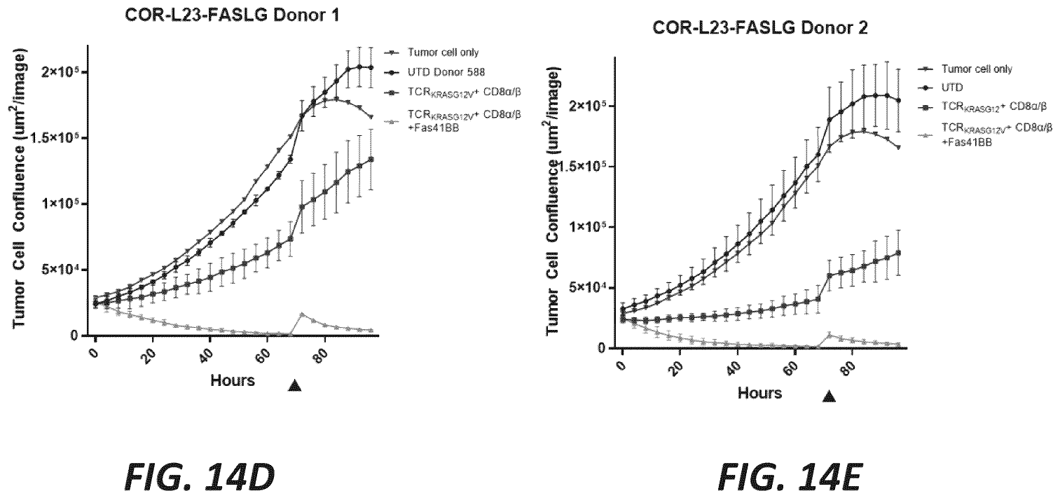

Further, FIGS. 14A-14D (FIGS. 14A-14D) demonstrate that a FAS-41BB fusion protein improved KRAS engineered T-cell tumor killing in vitro (e.g. cells expressing high levels of Fas ligand). In this experiment, CD4 and CD8 T cells comprising the TCR 11N4A against KRAS, CD8αβ co-receptor (e.g. exogenous CD8αβ co-receptor), and a FAS-41BB fusion protein according to SEQ ID NO: 80 (alongside the indicated controls) were co-cultured at 5:1 and 2:1effector:target cell ratios with SW527 tumor cells bearing the KRAS G12 mutation. As can be seen at the 2:1 condition, FAS-41BB fusion protein inclusion with KRAS TCRs improved killing of KRAS-positive tumor cells over just KRAS TCR alone. At the 2:1 target:effector ratio, large error bars indicate T cells losing tumor efficacy at different rates.

Untransduced T cells (UTD), T cells transduced with TCRKRASG12V+CD8α/β co-receptor or research-grade AFNT-211 T cells transduced with TCRKRASG12V, CD8α/β, and FAS-41BB were co-cultured with 1×104 HLA-A*11:01 SW620 tumor cells (A,B) or HLA-A*11:01 COR-L23 tumor cells (C,D) overexpressing FASLG and a NucLight Red fluorescent protein at a 5:1 effector:target ratio for up to 8 days. Cultures were restimulated approximately every 72 hours with equal numbers of tumor cells to mimic chronic antigen stimulation (▴). Two different donors were tested within the same study. Tumor confluence as measured by total NucLight Red object area is reported as a metric of tumor cell growth/viability throughout the study.

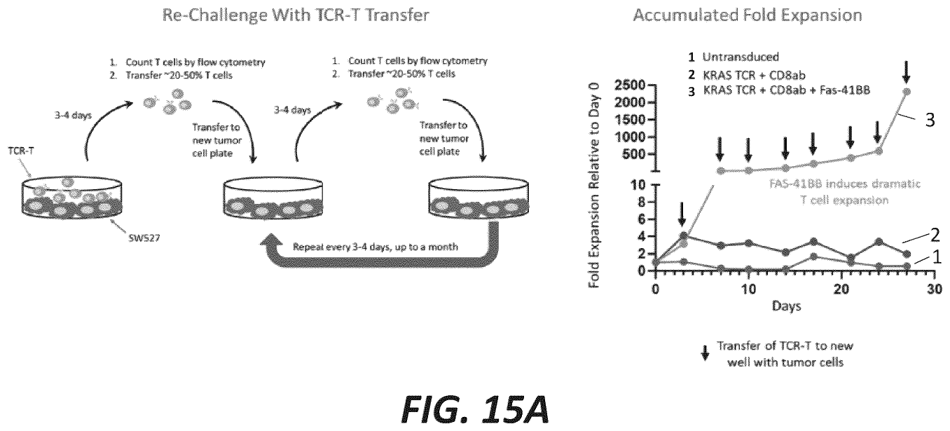

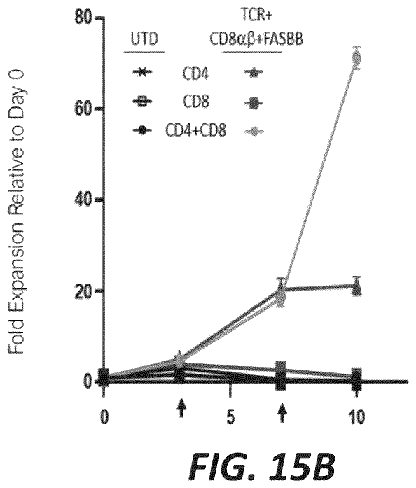

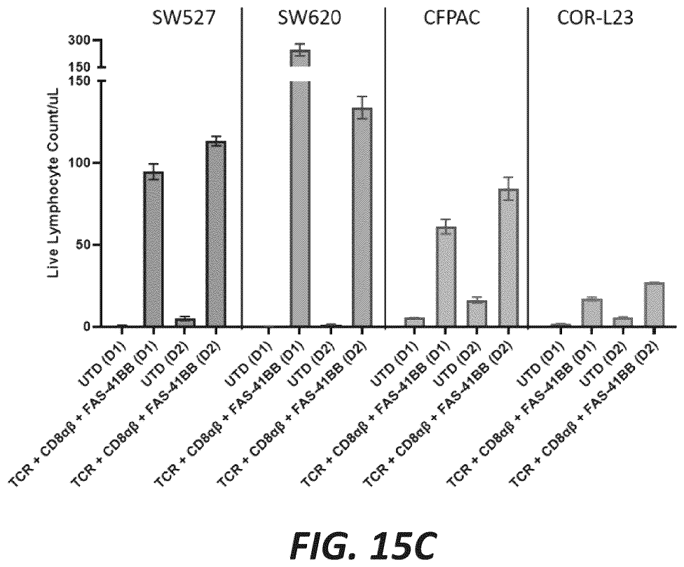

Additional in vitro experiments also demonstrated that a FAS-41BB fusion protein improved expansion of KRAS TCR bearing cells in an in vitro re-challenge assay as shown in FIG. 15A and FIG. 15B. The left panel of the figure is a scheme whereby T-cells comprising the TCR 11N4A against KRAS, CD8αβ co-receptor (e.g. exogenous CD8αβ co-receptor), and a FAS-41BB fusion protein according to SEQ ID NO: 80 (alongside the indicated controls) were co-cultured with SW527 cells for 3-4 days, followed by counting and transfer to a fresh cell plate of SW527 cells; repeating transfer to fresh plates of SW527 cells repeatedly as indicated. In the right panel is shown a graph of the expansion of the transferred T cells over time. As can be seen in the right panel graph, FAS-41BB fusion protein inclusion with KRAS TCRs improves proliferation of KRAS TCR bearing cells.

In addition, in FIG. 15B, an in vitro re-challenge assay was conducted to demonstrate that expansion of KRAS TCR-, CD8α/CD8β-, and FAS-41BB fusion protein-bearing cells was improved when the cells comprise both CD4⁺ and CD8⁺ T cells. Shown is a plot of accumulated fold expansion of CD4+), CD8+, CD4+/CD8+ mixture, or corresponding untransduced control primary T cells in co-culture with SW527 cell line expressing HLA-A*11:01 and KRAS mutant G12V. T cells were activated with anti-CD3/CD28 antibodies, either untransduced or lentivirally transduced with A11 G12V TCR+CD8αβ+FAS-41BB, expanded for 7 days, and cryopreserved. Frozen T cells were thawed and co-cultured with SW527 at an initial ratio of 1:1. Every 3-4 days (indicated by arrow), T cells were harvested from the culture, quantified by flow cytometry, and transferred to a secondary culture containing freshly plated SW527 tumor cells. Moreover, the TCR-engineered cells show improved proliferation rates relative to untransduced cells in response to endogenous processing and presentation of KRAS G12V antigen across a diverse panel of tumor cell lines (FIG. 15C).

### Example 10

### In Vivo Anti-Tumor Efficacy and Kaplan-Meier Survival Curve of Tumor-Bearing Mice Following Administration of Engineered CD4/CD8 T Cells with Addition of FAS/41BB Fusion Proteins

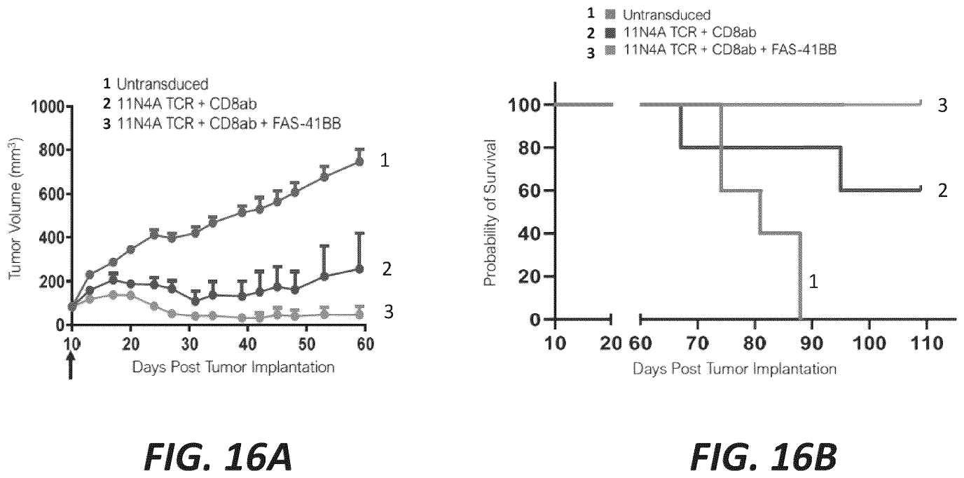

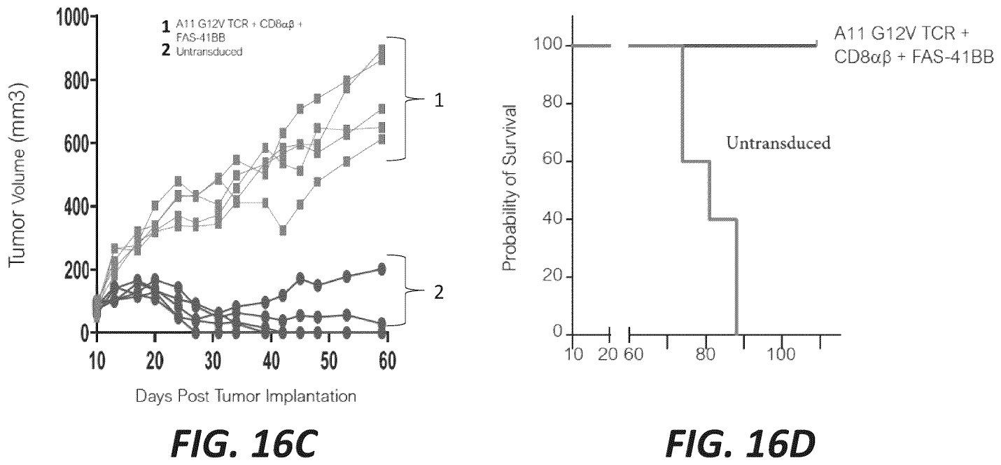

In vivo data as shown in FIG. 16A-FIG. 16D demonstrates that a FAS-41BB fusion protein improves therapeutic efficacy of cells expressing a KRAS TCR in an in vivo xenograft tumor model with SW527 cells. In this experiment, 10 million T cells comprising the TCR 11N4A against KRAS, CD8αβ co-receptor (e.g. exogenous CD8αβ co-receptor), and a FAS-41BB fusion protein (SEQ ID NO: 80) (alongside the indicated controls) were administered intravenously to immunodeficient mice bearing subcutaneous SW527 tumors, and tumor volume was measured over time. As shown in FIG. 16A, FAS-41BB fusion protein coexpression with KRAS TCRs improves killing of the SW527 tumors in vivo relative to that of the KRAS TCRs alone (FIG. 16A).

FIG. 16B is a Kaplan-Meier survival curve of mice bearing a SW527 xenograft model expressing HLA-A*11:01 and endogenous KRAS mutant G12V. Tumor-bearing mice received primary CD4/CD8 T cells that were either untransduced or lentivirally transduced with A11 G12V TCR+CD8αβ or A11 G12V TCR, CD8αβ co-receptor (e.g. exogenous CD8αβ co-receptor), and FAS-41BB and expanded for 7 days with anti-CD3/CD28 beads following transduction. 10 million transduced T cells were administered intravenously 10 days following SW527 subcutaneous inoculation when the tumor reached approximately 100 mm³. T cells were cryopreserved and thawed prior to administration.

In FIG. 16C, most mice achieved a complete response when treated with the engineered T cells disclosed that expressed a FAS-41BB fusion protein. In this experiment, primary CD4/CD8 T cells were lentivirally transduced with A11 G12V TCR, CD8αβ co-receptor (e.g. exogenous CD8αβ co-receptor), and FAS/41BB fusion protein. Transduced T cells were expanded for 7 days with ani-CD3/CD28 beads following transduction. Further, 10 million transduced T cells were administered intravenously 10 days following SW527 subcutaneous inoculation when the tumor reached approximately 100 mm³. After about a 60-day continuous measurement, most mice receiving T cells transduced with the A11 G12V TCR, CD8αβ co-receptor (e.g. exogenous CD8αβ co-receptor), and FAS-41BB achieved a complete reduction in tumor volume.

Cells transduced with TCR 11N4A, a CD8αβ co-receptor (e.g. exogenous CD8αβ co-receptor), and FAS/41BB fusion protein allow for longer survival of tumor-bearing mice versus mice administered untransduced cells. FIG. 16D is a Kaplan-Meier survival curve of mice bearing SW527 xenografts expressing HLA-A*11:01 and endogenous KRAS mutant G12V following administration of engineered CD4/CD8 T cells.. Tumor-bearing mice received primary CD4+/CD8+ T cells that either untransduced or lentivirally transduced with A11 G12V TCR, CD8αβ co-receptor (e.g. exogenous CD8αβ co-receptor), and FAS-41BB. Cells were expanded for 7 days with anti-CD3/CD28 beads following transduction. To initiate the experiment, 10 million transduced T cells were administered intravenously 10 days following SW527 cell subcutaneous inoculation when tumor reached approximately 100 mm³. T cells were cryopreserved and thawed prior to administration.

### Example 11

### Coordinated CD4/CD8 Response

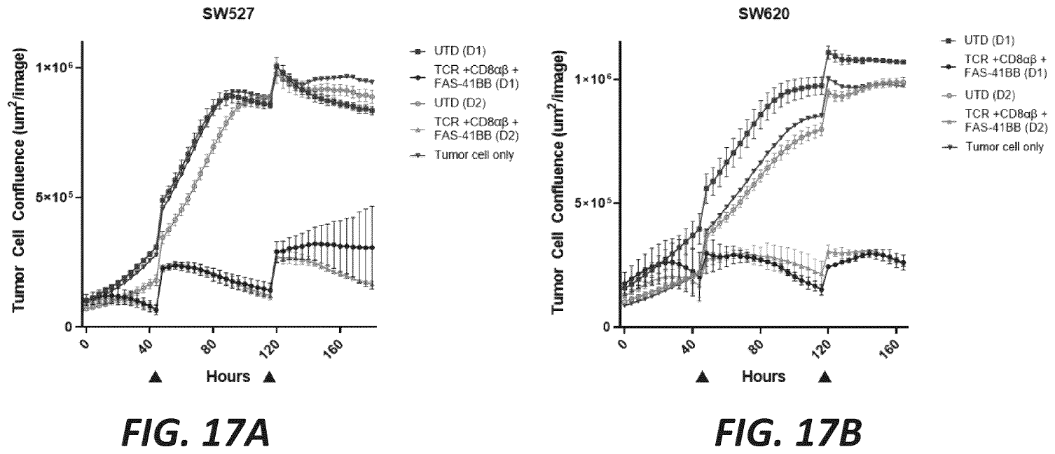

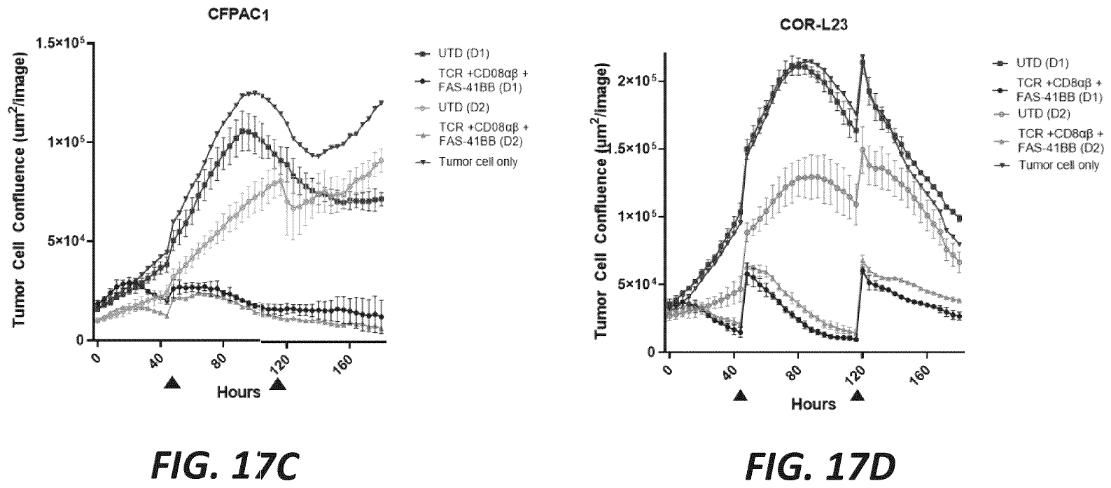

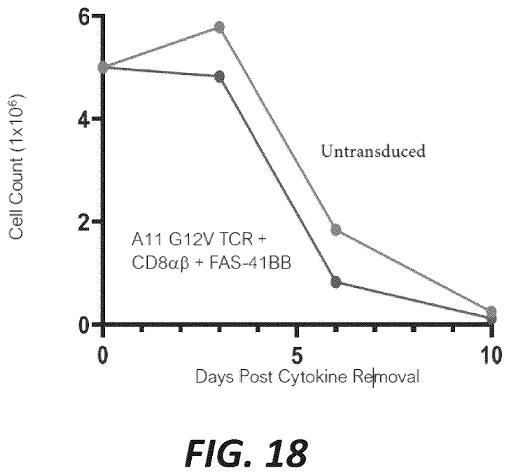

T cells lentivirally transduced to express a KRAS TCR, CD8αβ co-receptor (e.g. exogenous CD8αβ co-receptor), and a FAS-41BB fusion protein-have improved anti-tumor activity when they comprise both CD4⁺ and CD8⁺ T cells relative to CD4+ or CD8+ T cells alone. FIG. 17 is a plot of confluence of SW527 tumor cell line expressing a red fluorescent protein, HLA-A*11:01, and endogenous KRAS mutant G12V monitored in a live tumor-visualization assay quantifying red fluorescence signal over time. Cultures comprised a SW527 monoculture (“tumor cell alone”) or were co-cultured with untransduced CD4+/CD8+ mixed T cells, or CD4+, CD8+, or CD4+/CD8+ mixed T cells lentivirally transduced with A11 G12V TCR, CD8αβ co-receptor, FAS-41BB. Primary T cells were activated with anti-CD3/CD28 beads, expanded for 5 days following transduction, and co-cultured with SW527 cells at an initial ratio of 0.5:1. Every 3 days (indicated by arrow) additional fresh SW527 cells was added to the culture.

### Example 12

### Safety Profile of Primary T Cells Transduced with A11 G12V TCR+CD8αβ+FAS-41BB

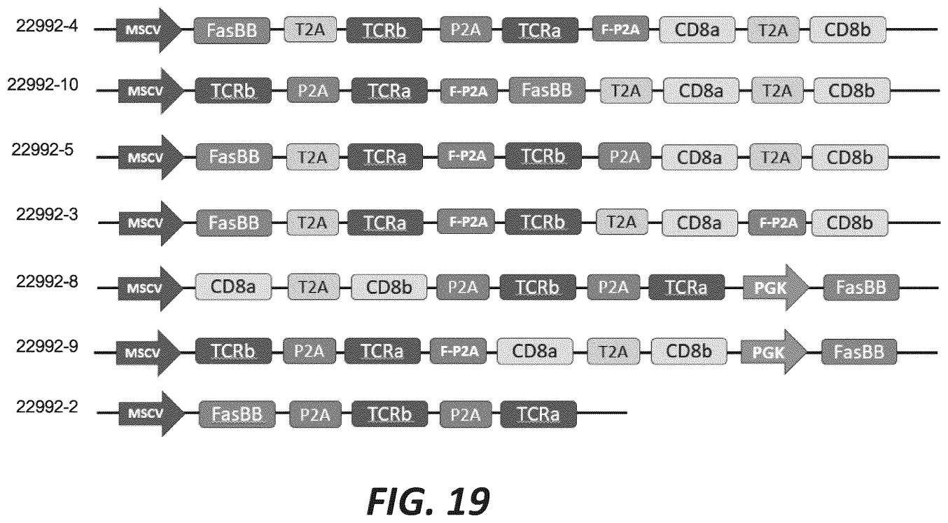

Cells transduced with TCR 11N4A, CD8αβ co-receptor (e.g. exogenous CD8αβ co-receptor), and a FAS/41BB fusion protein fail to proliferate in the absence of exogenous cytokine support, enhancing their safety profile. FIG. 19 is a plot of persistence (measured by cell count) of CD4+/CD8+ T cells monitored by quantifying cells every 2-4 days in absence of exogenous cytokines. Shown are primary T cells either untransduced (top line) or transduced with A11 G12V TCR, CD8αβ co-receptor, and FAS-41BB (bottom line) that have been expanded with anti-CD3/CD28 beads in media containing IL2/IL7/IL15 for 7-10 days and transferred to media without cytokine. Half of the media (without cytokine) was replenished every 2-4 days.

### Example 13

### Lentiviral Vector Design

Having established that T cells comprising both an anti-KRAS TCR (e.g., TCR 11N4A) and FAS/41BB fusion protein had superior qualities to those with just a TCR, designs for single lentiviral vectors comprising anti-KRAS TCR and FAS-41BB fusion protein (alongside CD8α/CD8β coreceptors) were executed (see e.g., FIG. 19). Most of the designs contemplated expressing anti-KRAS TCR (“TCRb” or “TCRa”), CD8α/CD8β (“CD8a” or “CD8b”), and FAS-41BB (“FasBB”) on a single translated RNA with the usage of in-frame sequences encoding self-cleaving peptides (“P2A,” “T2A,” “F-P2A”) separating regions encoding the separate polypeptides. It was contemplated that such constructs which have the multiple elements on a single vector or single cistron would have advantages in terms of manufacturing ease or cost, polypeptide expression, T cell therapeutic efficacy, or any combination of these things.

### Lentiviral Design Testing

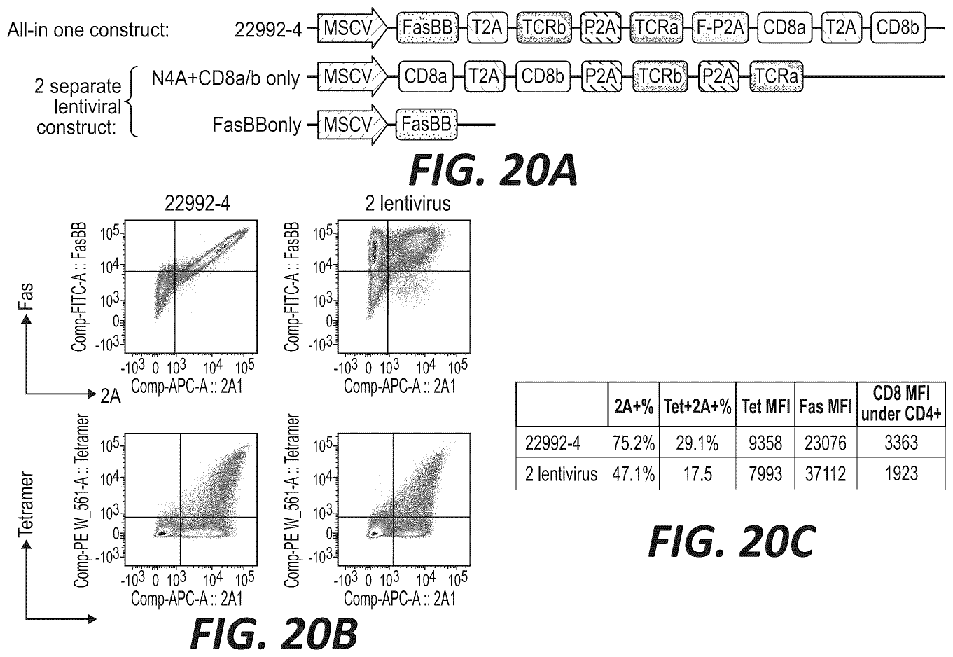

First, the performance of a manufacturing strategy that involves a single vector comprising anti-KRAS TCR, FAS-41BB fusion protein, and CD8α/CD8β was evaluated versus a strategy that involves anti-KRAS TCR and FAS-41BB fusion proteins on separate vectors (FIGS. 20A-20C). Lentiviral vectors were generated, and T cells transfected as described previously, and FACS analysis was performed to evaluate cells percentage of cells expressing a cistron comprising the anti-KRAS TCR (“2A+%”), percentage of cells expressing functional TCR and a cistron comprising the anti-KRAS TCR (“Tet+2A+%”), overall functional TCR expression (“Tet MFI”), FAS-41BB fusion protein expression (“Fas MFI”), and CD8α/CD8β coreceptor expression by CD4+ cells (“CD8 MFI under CD4+”). The FACS analysis indicated that, the single lentiviral strategy (“22992-4”) and the dual lentiviral strategy (“2 lentivirus”) were both able to express TCR and CD8α/CD8β transgenes.

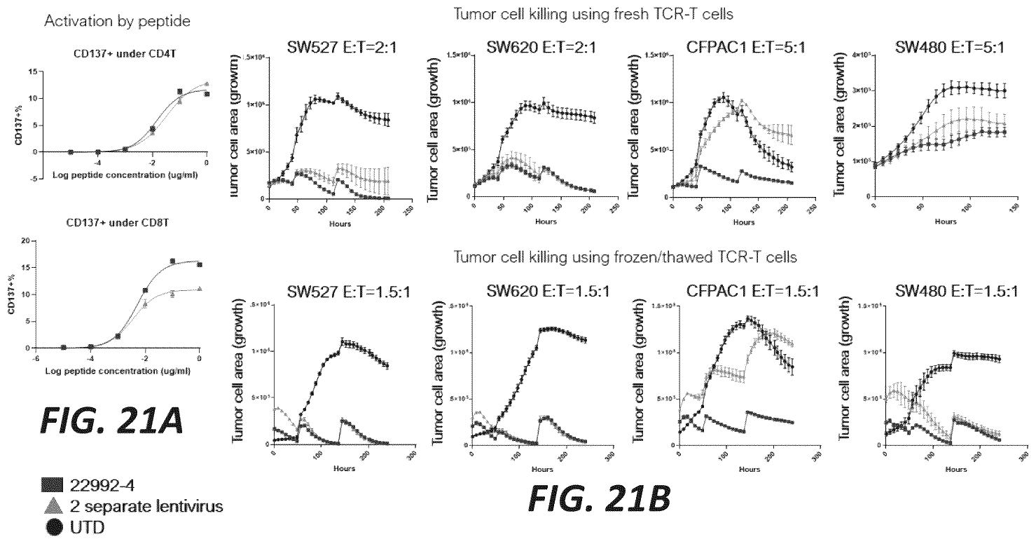

After transfecting the T cells, the cells comprising anti-KRAS TCR, FAS-41BB fusion protein, and CD8α/CD8β on a single construct (“22992-4”) were evaluated versus cells comprising anti-KRAS TCR and FAS-41BB fusion protein (“2 lentivirus”) in terms of activation by antigenic peptide (FIG. 21A) and tumor cell killing (FIG. 21B). Consistent with the superior expression, cells transfected with the single lentiviral vector (“22992-4”) were equivalent or superior to the dual lentiviral vector (“2 lentivirus”).

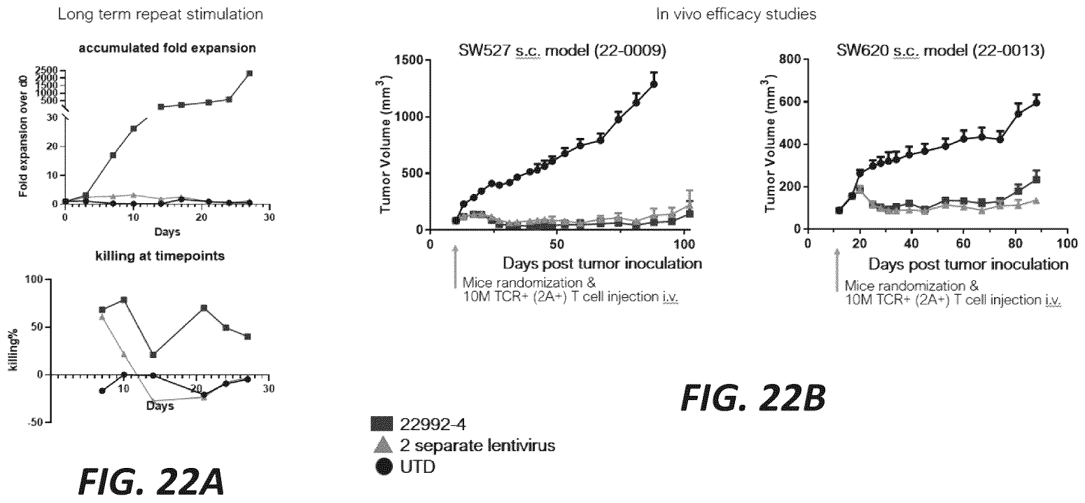

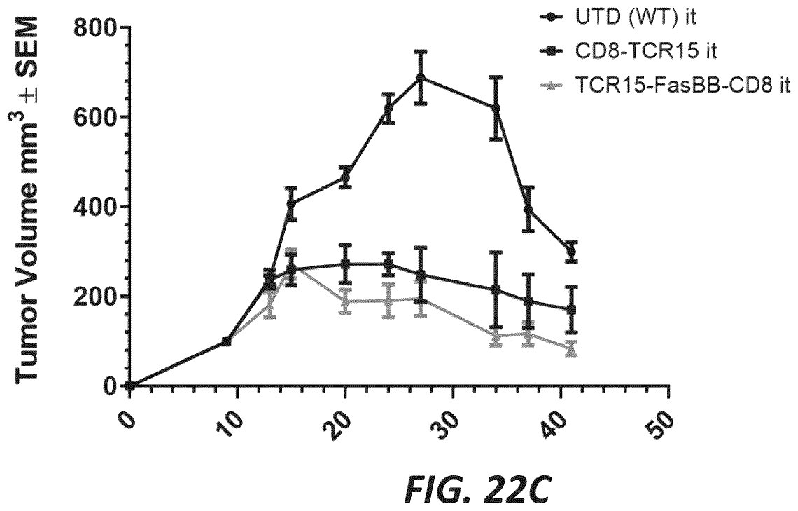

Similarly transfected cells were also evaluated in terms of repeat stimulation and cell killing (FIG. 22A) and in vivo efficacy in a xenograft model (FIG. 22B) as previously described. Consistent with the superior expression, cells transfected with the single lentiviral vector (“22992-4”) were equivalent or superior to the dual lentiviral vector (“2 lentivirus”) in these evaluations.

CD4+ and CD8+ T cells transfected with lentiviral vector encoding an anti-KRAS G12D TCR, a Fas-41BB fusion protein, and a CD8αβ co-receptor (e.g. exogenous CD8αβ co-receptor) also displayed in vivo efficacy in a xenograft model (FIG. 22C).

### Example 14

### Clinical Development Plan (Prophetic)

A First-in-Human (FIH), single-arm, open-label, multi-center Phase I study comprising a dose finding part followed by a dose expansion part to evaluate the safety, tolerability, and preliminary anti-tumor efficacy of cells transduced with TCR 11N4A, CD8αβ co-receptor (e.g. exogenous CD8αβ co-receptor), and a FAS/41BB fusion protein will be evaluated as an autologous, HLA-A*11:01-restricted KRASG12V targeting TCR T cell therapy in subjects with advanced or metastatic solid tumors. To be eligible, subjects are positive for the KRASG12V mutation (e.g., via a KRAS sequencing or genotyping test) in the tumor and present with an HLA-A*11:01 allele.

### Manufacturing for Lentiviral Vector

The lentiviral vector encoding HLA-A*11:01, KRASG12V-specific TCRα/β, FAS-41BB fusion protein and the CD8α/β coreceptor is a key drug substance intermediate (DSI) used in the manufacturing process. DSI is manufactured under cGMP conditions and comprises a plasmid encoding the HLA-A*11:01, KRASG12V-specific TCRα/β, FAS-41BB and the CD8a/p coreceptor (in that order, except that the beta chain of the KRAS TCR is upstream from the alpha chain). The lentiviral vector will be produced using a transient transfection process.

### Cell Transduction (Function) Titer Evaluation

The lentiviral vector (LVV) transduction titer (reported in TU/mL) is used to calculate the volume of LVV required for the transduction of patient T cells to achieve the targeted multiplicity of infection (MOI).

### Potency by Expression

Jurkat E6-1 cells do not express endogenous CD8 and have been additionally disrupted for endogenous TCRα and TCRβ expression (double knockout of the TRAC and TRBC loci) for the evaluation of LVV-driven TCR expression. Transduced cells are cultured for 3 days and then subject to fluorescent antibody staining and flow cytometry analyses to evaluate the expression of transduced CD8 chains. CD8 chains are the components of the resultant transgene cassette encoded by the LVV and therefore their expression can be considered a surrogate for expression of the upstream transgenes (TCRα, TCRβ and FAS-41BB).

### Vector Genome (Genomic) Titer Evaluation

LVV genome titer is measured using Reverse Transcription-mediated droplet digital PCR (RT-ddPCR) to determine the number of LVV genome copies present per unit volume. Encoded transgenes are codon-optimized and can be distinguished from their cellular counterparts. A primer/probe set was designed to detect and quantify nucleic acid sequences, specific to the TCRα codon-optimized nucleic acid sequences. Results are reported as vector genomes per mL (VG/mL). Physical titer (P24) will also be analyzed as part of characterization.

TABLE 1
The Proposed Phase I Specification for Lentiviral Vector

| | | |
|---|---|---|
| Test | Method | Reportable |

Potency

| | | |
|---|---|---|
| Transduction | Cell based assay | TU/mL |
| (functional) Titer | | |
| Potency by Expression | Flow Cytometry | % CD8+ |

Strength

| | | |
|---|---|---|
| Genomic to Functional | Calculation | Ratio (VG/TU) |
| Ratio | | |
| LVV Genome (genomic) | RT-ddPCR | VG/mL |
| Titer | | |

Identity

| | | |
|---|---|---|
| Proviral Sequencing¹ | Sanger Sequencing | % Sequence match |

Safety

| | | |
|---|---|---|
| Sterility | USP <71>/Ph. Eur | Free from viable |
| | 2.6.1 | bacterial and fungal |
| | | contamination |
| Endotoxin | USP <85>/Ph. Eur | EU/mL |
| | 2.6.14 | |
| Mycoplasma² | USP <63>/Ph. Eur | No evidence of |
| | 2.6.7 | mycoplasma |
| | | contamination |
| RCL³ | C8166 cell assay/Q- | No RCL detected |
| | PERT | |
| Adventitious Virus | Cell based/ | No Cytopathic effect |
| Agents⁴ | microscopically | or hemadsorption |
| | | observed |

Purity⁵

| | | |
|---|---|---|
| Residual Nuclease | ELISA | pg/mL of nuclease |
| Residual plasmid DNA | ddPCR | Copies/μL of plasmid |
| | | DNA |
| Residual Host Cell DNA | ddPCR | pg/μL of genomic |
| | | DNA |
| Residual Host Cell | ELISA | ng/mL of HCP |
| Protein | | |
| Ad5 E1A | ddPCR | pg/μL of genomic |
| | | DNA |
| Appearance | Visual inspection | Color & turbidity |
| pH | USP <791>/Ph. Eur | pH |
| | 2.2.3 | |
| Osmolality | USP <785>/Ph. Eur | mOsmol/kg |
| | 2.2.35 | |

Therapeutic Cell Transduction and Expansion
¹Testing performed on purified bulk
²Testing performed on bulk harvest
³RCL testing is performed on both viral supernatant as well as end of production cells
⁴Testing performed on bulk harvest
⁵Testing performed on purified bulk

Cells prepared by leukapheresis from a patient are stored via controlled rate freezing in liquid nitrogen until use. On Process Day 0, the cryopreserved apheresis is thawed and positively selected first for CD8-expressing T cells using immunomagnetic beads; the flow through is then positively selected for CD4-expressing T cells. The CD8+ and CD4+ selected T cells are combined at a fixed CD4:CD8 ratio, activated with CD3/CD28-specific antibodies, and cultured in serum-free media supplemented with serum replacement and cytokines. Activated cells are then incubated overnight at 37° C. and 5% CO2. On the following day, T cells are transduced with the LVV, combined with a chemical transduction enhancer, and again incubated at 37° C. and 5% CO₂. The cells are expanded, formulated, and cryopreserved.

### Flow Cytometry

Flow cytometry is used to evaluate A11G12V TCR expression frequency, transduction frequency and T cell purity of the therapeutic cell formulation using staining of CD3, CD4, CD8, Dextramer (comprised of single chain monomers attached to a flexible dextran backbone which is fluorescently conjugated) specific to A11G12V TCR.

### TCR Expression Frequency for Potency and Dose Calculation

TCR detection is performed by Dextramer® reagent staining (fluor-conjugated A11 MHC complexed with KRAS G12V peptide and multimerized via biotin-streptavidin interactions) to detect the expression and structural functionality of the TCR on the cell surface. Assay controls include untransduced healthy donor cells (negative reference control) which provide a baseline measure and demonstrate specificity. The percent of TCR+ cells (vial dextramer staining) are used for the dose calculation (Dose=total viable cell count*% Dextramer+of CD3+ cells).

### Cytokine Secretion

As a further measurement of potency, cytokine secretion can be evaluated to demonstrate engineered T cell functionality. The production of specific cytokines is observed as a consequence of T cell activation; interferon γ (IFNγ) is a widely accepted biomarker of activated T-cells. DP cells are co-cultured with HLA-matched antigen presenting cells (APC) and loaded with KRAS G12V peptide. Untransduced cells are included as negative control. Co-culture of DP cells with peptide loaded APC cells provides a relevant tissue culture platform to assess T cell activation signaling. Following co-culture, the supernatant is collected and measured for IFNγ concentration using immunological methods.

### Identity by PCR

To ensure drug product identity, genomic DNA is extracted from post LVV-integrated DP cells. The DNA is isolated, normalized and then evaluated using primer/probe sets specific for the encoded transgenes. Additionally, both positive and negative controls are evaluated in parallel to assure assay performance.

### Vector Copy Number

Vector copy number (VCN) is determined using droplet digital PCR (ddPCR) to quantify the number of proviral integrated DNA copies per host cell genome. VCN is determined by ddPCR using multiplex primer/probe sets against the WPRE (LVV backbone) and RPPH1 (RNaseP; genome reference) cassettes in drug products. The resulting VCN is normalized to the transduction frequency.

### Replication-Competent Lentivirus

The presence or absence of replication competent lentivirus (RCL) is determined using a droplet digital polymerase chain reaction (ddPCR) assay. This ddPCR assay is used to detect the gene sequence for the vesicular stomatitis virus G (VSV-G) envelope protein of the lentiviral vector as an indicator of RCL in the test sample. Results are reported as detected or not detected for the presence of the target gene (VSV-G) in the test sample.

TABLE 2
The Proposed Phase I Specification
for Therapeutic Cell Formulation

| | | |
|---|---|---|
| Test | Method | Reportable |

Potency

| | | |
|---|---|---|
| Transgene expression | Flow Cytometry | % Dextramer+ (of CD3+) |
| frequency | | |
| Cytokine Secretion | Co-culture and | IFNγ (pg/mL) |
| | Immunoassay | |

Identity

| | | |
|---|---|---|
| Identity by PCR | ddPCR | Codon Optimized TCRα |

Safety

| | | |
|---|---|---|
| Rapid Sterility | BacT/Alert | Colony Growth |
| Endotoxin | USP <85>/Ph. Eur. | EU/mL |
| | 2.6.14 | |
| Rapid Mycoplasma⁶ | Reverse | Sequence presence |
| | transcription- | |
| | multiplex PCR | |
| Rapid RCL | ddPCR | VSV-G sequence presence |

Purity

| | | |
|---|---|---|
| T-cell Purity | Flow Cytometry | % CD3+ |
| Viability | Automated Cell | % Viable |
| | Counter | |

Quality

| | | |
|---|---|---|
| Appearance | Visual inspection | Color and turbidity |

Strength

| | | |
|---|---|---|
| Dose | Flow Cytometry | Total Viable cell count * % |
| | | Dextramer+ (of CD3+ |
| | | cells) |

### Dose Finding/Escalation Study For Cell Therapy (Prophetic)

Dose finding/escalation of cells transduced with TCR 11N4A, CD8αβ co-receptor (e.g. exogenous CD8αβ co-receptor), and a FAS/41BB fusion protein is guided by the Bayesian optimal interval phase I/II (BOIN12) trial design (Lin et al., (2020) JCO Precision Oncology, 4, PO.20.00257; doi.org/10.1200/PO.20.00257) to find the optimal biological dose (OBD). The BOIN12 design uses utility to quantify the desirability of a dose in terms of “toxicity-efficacy tradeoffs”, and adaptively allocates subjects to the dose that has the highest estimated desirability. The OBD will be selected as the dose that is admissible and has the highest estimated utility based on the isotonic estimation method described in Lin et al. A total sample size of up to 20 subjects is enrolled in dose finding/escalation. Staggering of at least 28 days between subject 1 and subject 2 of each new dose level is required. Each subject of the previous cohort completes the full dose limiting toxicity (DLT) observation period of 28 days before a new, not previously assessed dose level cohort can enter the treatment and active care period which is defined as the period between start of the first day of lymphodepleting chemotherapy (LDC) and end of day 28 after cell Investigational Medicinal Product administration (i.e., the DLT observation period). To prevent assignment of subjects to toxic and/or futile doses, two dose acceptability criteria are used by BOIN12 to decide which doses may be used to treat subjects.

To ensure the safety of study subjects, the following criteria must be met prior to start of the treatment and active care period, i.e., from start of lymphodepleting chemotherapy to end of DLT observation period.
Eastern Cooperative Oncology Group (ECOG) performance status 0-1
Adequate organ and marrow function
Female subjects of childbearing age have a negative serum pregnancy test within 14 days prior to LDC

Any cytotoxic chemotherapy, investigational agents, or any anti-tumor drug from a previous treatment regimen or clinical study is stopped 5 half-lives or 14 days (whichever comes first) prior to start of the treatment and active care period. The same rule applies to the administration of bridging therapy if permitted in the protocol.

Study subjects are closely monitored for adverse events by trained medical staff. Blood samples to monitor cytokine levels are collected at regular intervals and ad-hoc based on the clinical presentation of a subject. Anti-microbial prophylaxis is administered to subjects as per institutional guidelines.

Grading of adverse events is performed in accordance with NCI CTCAE version 5.0. For immune effector cell-associated neurotoxicity syndrome (ICANS) and cytokine release syndrome (CRS), the ASTCT Consensus Grading is used (Lee et al., (2019) Biology of Blood and Marrow Transplantation: Journal of the American Society for Blood and Marrow Transplantation, 25(4), Article 4; doi.org/10.1016/j.bbmt.2018.12.758.) If applicable, the DLT assessment period is extended to follow ongoing AEs until resolution of the event or confirmation that the event is a DLT.

The DLTs are defined as follows:
1. Any treatment emergent Grade 4 or 5 CRS
2. Any treatment emergent Grade 3 CRS that does not resolve to Grade 2 within 7 days
3. Grade 3 or higher neurotoxicity that does not resolve to Grade 2 within 72 hours
4. Grade 3 or greater allergic reactions related to cell infusion
5. Any treatment-emergent autoimmune toxicity ≥Grade 3
6. Grade 3 or greater organ toxicity (cardiac, dermatologic, gastrointestinal, hepatic, pulmonary, renal/genitourinary), not pre-existing or not due to the underlying malignancy occurring within 30 days of cell infusion.
7. Any Grade 3 or higher non-hematologic toxicity should resolve to Grade 2 or less within 7 days
8. Grade 3 thrombocytopenia with bleeding or Grade ≥hematologic toxicities that fail to recover to ≤Grade 2 within 7 days
9. Any other clinically significant toxicity related to cell therapy not meeting above criteria that is deemed by the investigator to represent a DLT.

The following conditions are not considered DLTs:
Grade 3 fatigue
Grade 3 endocrine disorder (thyroid, pituitary, and/or adrenal insufficiency) that is managed with or without systemic corticosteroids and/or hormonal replacement therapy with resolution of symptoms
Grade 3 hypertension that can be controlled with medical therapy
Grade 3 lab value abnormalities that are asymptomatic and clinically insignificant
Vitiligo or alopecia of any AE grade Subjects who receive cells transduced with TCR 11N4A, CD8αβ co-receptor (e.g. exogenous CD8αβ co-receptor), and a FAS/41BB fusion protein and have a confirmed partial response (PR) on imaging may receive a second infusion of the cells, at the investigator's discretion. Subjects who achieve a transient complete response (CR) and later progressed within the short-term follow-up (STFU) period of this study may also be considered for re-treatment at the investigator's discretion. Additionally, subjects need to have tolerated the initial infusion of cells transduced with TCR 11N4A, CD8αβ co-receptor (e.g. exogenous CD8αβ co-receptor), and a FAS/41BB fusion protein without occurrence of any DLTs. Re-treatment is administered at the same dose level as the first infusion of the cells. In cases of limited cell product availability for re-treatment, a lower dose is considered after discussion with the medical monitor. No LDC is administered if subjects receive the second dose of cells transduced with TCR 11N4A, CD8αβ co-receptor (e.g. exogenous CD8αβ co-receptor), and a FAS/41BB fusion protein within 2 months after the first dose. If re-treatment occurs more than 2 months after the first infusion of the cells, the decision to administer LDC is left to the investigator. However, re-administration of LDC prior to a second infusion of the TCR-engineered cells follows the LDC inclusion criteria described in the draft protocol synopsis. AEs following re-treatment with the cells are collected and reported but are not used in the DLT analysis.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A polynucleotide comprising a nucleic acid sequence encoding:
(a) a binding protein, wherein the binding protein comprises:
a T cell receptor (TCR) or a functional derivative thereof; or
a chimeric antigen receptor (CAR) or a functional derivative thereof; and
(b) a fusion protein, wherein the fusion protein comprises:
(i) an extracellular component comprising a CD95 ligand (FasL) binding domain that comprises a CD95 (Fas) ectodomain or a functional fragment thereof; and
(ii) an intracellular component comprising a CD137 (4-1BB) intracellular signaling domain, wherein the nucleic acid sequence encoding the binding protein is positioned upstream of the nucleic acid sequence encoding the fusion polypeptide.

2. The polynucleotide of claim 1,
further comprising a nucleic acid sequence encoding:
(c) a CD8 co-receptor α or β chain or a portion or variant thereof, wherein the sequence encoding the binding protein is positioned upstream of the sequence encoding the extracellular portion of a CD8 co-receptor α or β chain or the portion or variant thereof; and/or
further comprising a nucleic acid sequence encoding:
(c) a CD8 co-receptor α and β chain or portions or variants thereof, wherein the sequence encoding the binding protein is positioned upstream of the sequence encoding the extracellular portion of the CD8 co-receptor α and β chains or the portions or variants thereof.

3. The polynucleotide of claim 2, wherein the nucleic acid sequence encoding the fusion protein further encodes:
(d) a hydrophobic component between the extracellular and intracellular components of the fusion protein.

4. The polynucleotide of claim 1,
wherein the binding protein comprises a binding domain that binds to a peptide:HLA complex, wherein the complex comprises a neoantigen peptide and an HLA protein;
wherein the binding protein comprises a single-chain TCR (scTCR) or a single-chain T cell receptor variable fragment (scTv);
wherein the binding protein comprises a TCR α chain variable (Vα) domain or a TCR β chain variable (Vβ) domain; OR
wherein the binding protein comprises a TCR α chain variable (Vα) domain and a TCR β chain variable (Vβ) domain.

5. The polynucleotide of claim 1, wherein the CD95 (Fas) ligand binding domain is a Fas ectodomain or a functional fragment thereof and/or wherein the intracellular component is a CD137 (4-1BB) transmembrane domain or a functional fragment thereof.

6. The polynucleotide of claim 1, wherein the neoantigen peptide is a KRAS, HRAS, NRAS, p53, or PIK3CA mutant peptide.

7. The polynucleotide of claim 1, wherein the KRAS mutant peptide comprises x-V-G-A-x-G-x-x-K, wherein x denotes any amino acid; and wherein the HLA protein is encoded by an HLA-A*11 or HLA-A*11:01 allele.

8. The polynucleotide of claim 2,
further comprising a nucleic acid sequence encoding a self-cleaving peptide between the nucleic acid sequence encoding the TCR receptor variable α (Vα) region and the nucleic acid sequence encoding the TCR receptor variable β (Vβ) region further comprising a nucleic acid sequence encoding a self-cleaving peptide disposed between (a) and (b) or, where (c) is present, (b) and (c);
further comprising a nucleic acid sequence encoding a self-cleaving peptide between the sequence encoding the CD8 co-receptor α chain and the sequence encoding the CD8 co-receptor β chain
further comprising a nucleic acid sequence that encodes a self-cleaving peptide that is disposed between the nucleic acid sequence encoding a binding protein and the nucleic acid sequence encoding a polypeptide comprising an extracellular portion of a CD8 co-receptor α chain; and/or the nucleic acid sequence encoding a binding protein and the nucleic acid sequence encoding a polypeptide comprising an extracellular portion of a CD8 co-receptor β chain.
further comprising, operably linked in-frame:
(iii)(pnBP)-(pnSCP₁)-(pnCD8α)-(pnSCP₂)-(pnCD8β)-(pnFP); or
(iv)(pnBP)-(pnSCP₁)-(pnCD8β)-(pnSCP₂)-(pnCD8α)-(pnFP);
(iii)(pnBP)-(pnSCP₁)-(pnFP)-(pnSCP₁)-(pnCD8α)-(pnSCP₂)-(pnCD8β); or
(iv)(pnBP)-(pnSCP₁)-(pnFP)-(pnSCP₁)-(pnCD8β)-(pnSCP₂)-(pnCD8α);
wherein pnCD8α is the nucleic acid sequence encoding a polypeptide that comprises an extracellular portion of a CD8 co-receptor α chain,
wherein pnCD8β is the nucleic acid sequence encoding a polypeptide that comprises an extracellular portion of a CD8 co-receptor α chain,
wherein pnBP is the nucleic acid sequence encoding a binding protein,
wherein pnFP is the nucleic acid sequence encoding a fusion protein, and
wherein pnSCP₁ and pnSCP₂ are each independently a polynucleotide encoding a self-cleaving peptide, wherein the polynucleotides and/or the encoded self-cleaving peptides are optionally the same or different.

9. The polynucleotide of claim 8, wherein the self-cleaving peptide is a P2A, T2A, E2A, or a furin peptide.

10. A vector comprising the polynucleotide of claim 1.

11. A host cell comprising the polynucleotide of claim 1.

12. A method for treating a disease or disorder associated with a KRAS G12V mutation or a NRAS G12V mutation or a HRAS G12V mutation in a subject, the method comprising administering to the subject an effective amount of the host cell of claim 11.

13. A method of eliciting an immune reaction against a cell expressing a neoantigen, the method comprising contacting the cell with the cell comprising the polynucleotide of claim 1.

14. A method of eliciting an immune reaction against a cell expressing a neoantigen, the method comprising contacting the cell with the host cell of claim 11.

15. A method of genetically engineering an immune cell, the method comprising contacting the cell with a polynucleotide comprising a nucleic acid sequence encoding a T cell receptor (TCR) or functional fragment or variant thereof, a CD8α and/or a CD8β co-receptor or functional fragment or variant thereof, and a fusion protein comprising a CD95 (Fas) ectodomain or a functional fragment thereof and an intracellular component comprising a CD137 (4-1BB) intracellular signaling domain, and expanding the immune cell.

16. A host cell comprising:
(a) a fusion protein, wherein the fusion protein comprises:
(i) an extracellular component comprising a CD95 ligand (FasL) binding domain that comprises a CD95 (Fas) ectodomain or a functional fragment thereof; and
(ii) an intracellular component comprising a CD137 (4-1BB) intracellular signaling domain, wherein the nucleic acid sequence encoding the binding protein is positioned upstream of the nucleic acid sequence encoding the fusion polypeptide; and
(b) an exogenous CD8 co-receptor α or β chain or a portion or variant thereof.

17. A method for treating a cancer in a subject, comprising administering to the subject an effective amount of the host cell of claim 11.

18. A composition comprising a plurality of host cell, wherein the host cells comprise T-cells directed against a mutant KRAS peptide wherein the composition:
(a) comprises at least 10%, 15%, 20%, 25%, 30%, 35%, 40%, 45%, 50%, 55%, 60%, 65%, 70%, 75%, 80%, 85%, 90%, or greater CD3⁺ cells that stain with dextramer specific for mutant KRAS peptide as assessed by flow cytometry;
(b) comprises at least 80%, 85%, 90%, 92%, 94%, 96%, 98%, or greater T cells that are CD3-positive as assessed by flow cytometry;
(c) comprises at least 70%, 75%, 80%, 85%, 90%, or greater viable cells as assessed by automated cell counting.

19. A composition comprising the host cells of claim 11 and a pharmaceutically acceptable excipient.

20. A composition comprising the polynucleotide of claim 1 and a pharmaceutically acceptable excipient.


SEQUENCE LISTING (1071 sequences)

SEQ ID NO: 1 - protein, 189 aa, synthetic construct [sequence omitted]

SEQ ID NO: 2 - protein, 10 aa, synthetic construct
VVVGAVGVGK

SEQ ID NO: 3 - protein, 9 aa, synthetic construct
VVGAVGVGK

SEQ ID NO: 4 - protein, 9 aa, Homo sapiens
  VARIANT 7: note = N can be any amino acid
  VARIANT 1: note = N can be any amino acid
  VARIANT 8: note = N can be any amino acid
  VARIANT 5: note = N can be any amino acid
NVGANGNNK

SEQ ID NO: 5 - DNA, 843 bp, unidentified
  source 1..843: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagtggggtttcaggaaacacacctcttgtctttggaaagggcacaagactttctgtgattgcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 6 - DNA, 930 bp, unidentified
  source 1..930: mol_type = other DNA
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcgtcgggactgtggagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 7 - DNA, 1836 bp, synthetic construct
  source 1..1836: mol_type = other DNA
atgggctctagactgttgtgttgggttctgctgtgtctgcttggagctggacctgtgaaagctggagttacccagacacccagatatctgatcaagaccagaggacagcaggtgacactgagctgtagccctatttctggccacaggagcgttagctggtatcagcaaacacccgggcagggactacaatttctattcgagtacttcagcgagacccagcggaataagggcaattttcctggcagatttagcggcaggcagttcagcaacagcagaagcgagatgaacgtgagcaccctggaattaggcgattctgctctgtacctgtgtgcctcttctgtgggaacagtggagcagtactttggccccggcacgagactgacagtgacagaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggccatgttactaggagcgagcgtgctgattctgtggttacagcctgattgggtgaactctcagcagaagaacgacgatcagcaggtgaagcagaatagcccctctctgtctgtgcaggagggcagaatctctatcctgaattgcgactacaccaacagcatgttcgactattttctgtggtacaaaaaataccccgccgagggccctacattcctgatcagcatcagctctatcaaggacaagaacgaggatggcagatttaccgtgttcctgaacaagagcgccaagcacctgagcctgcacattgtgccttctcaacctggcgattctgctgtgtacttttgtgctgcctctggagtgagcggcaatacacctctagtgttcgggaagggcacaagactgtctgttattgcaaacattcaaaaccccgaccctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 8 - DNA, 1836 bp, synthetic construct
  source 1..1836: mol_type = other DNA
atgggatctagattgctttgttgggtgctgctgtgcctgctcggagccggacctgtgaaagctggcgttacccagacacctagatacctgatcaagaccagaggccagcaagtgaccctgagctgctctcctatcagcggccacagaagcgtgtcctggtatcagcagacacctggacagggcctgcagttcctgttcgagtacttcagcgagacacagcggaacaagggcaacttccccggcagattttccggcagacagttcagcaacagccgcagcgagatgaacgtgtccacactggaactgggcgacagcgccctgtatctgtgtgcctcttctgtgggcaccgtggaacagtactttggccctggcaccagactgaccgtgaccgaggatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtttctgccctggtgctgatggccatggtcaagcggaaggattctagaggcggatccggagccaccaacttcagcctgcttaaacaggccggcgacgtggaagagaaccctggacctatggctatgctgctgggagcctctgtgctgatcctgtggctgcaacccgattgggtcaacagccagcagaagaacgacgaccagcaagtcaagcagaacagccccagcctgagcgtgcaagagggcagaatcagcatcctgaactgcgactacaccaactctatgttcgactactttctgtggtacaagaagtaccccgccgagggacccaccttcctgatcagcatcagcagcatcaaggacaagaacgaggacggccggttcaccgtgtttctgaacaagagcgccaagcacctgagcctgcacatcgtgccttctcagcctggcgatagcgccgtgtacttttgtgctgccagcggcgtgtcaggcaacacccctctggtttttggcaagggcacacgcctgtccgtgatcgccaacattcagaaccctgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagtccgatttcgcctgcgccaacgccttcaacaacagcattatccccgaggacacattcttcccaagtcctgagtccagctgcgacgtgaagctggtggaaaagagcttcgagacagacaccaacctgaacttccagaatctgagcgtgatcggcttcagaatcctgctgctgaaggtggccggattcaacctgctgatgaccctcagactgtggtccagctga

SEQ ID NO: 9 - DNA, 3300 bp, synthetic construct
  source 1..3300: mol_type = other DNA
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggcagcggagaaggcagaggctccctgcttacatgcggcgacgtggaagagaaccccggacctatgaggcctagactgtggctgcttctggctgcccagctgacagtgctgcacggcaattctgtcctgcagcagacccctgcctacatcaaggtgcagaccaacaagatggtcatgctgagctgcgaggccaagatcagcctgtccaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaacgcgtgtgcagactgcccaggcctgagacacaaaagggccctctgtgcagccctatcacactgggattgctggtggctggcgttctggtcctgctggtgtctctgggagttgccatccacctgtgctgtagaagaaggcgggccagactgcggttcatgaagcagttctacaaaggcagcggcgccaccaacttcagcctgctgaaacaagccggcgacgtcgaggaaaatcctggacctatgggctctagactgttgtgttgggttctgctgtgtctgcttggagctggacctgtgaaagctggagttacccagacacccagatatctgatcaagaccagaggacagcaggtgacactgagctgtagccctatttctggccacaggagcgttagctggtatcagcaaacacccgggcagggactacaatttctattcgagtacttcagcgagacccagcggaataagggcaattttcctggcagatttagcggcaggcagttcagcaacagcagaagcgagatgaacgtgagcaccctggaattaggcgattctgctctgtacctgtgtgcctcttctgtgggaacagtggagcagtactttggccccggcacgagactgacagtgacagaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggccatgttactaggagcgagcgtgctgattctgtggttacagcctgattgggtgaactctcagcagaagaacgacgatcagcaggtgaagcagaatagcccctctctgtctgtgcaggagggcagaatctctatcctgaattgcgactacaccaacagcatgttcgactattttctgtggtacaaaaaataccccgccgagggccctacattcctgatcagcatcagctctatcaaggacaagaacgaggatggcagatttaccgtgttcctgaacaagagcgccaagcacctgagcctgcacattgtgccttctcaacctggcgattctgctgtgtacttttgtgctgcctctggagtgagcggcaatacacctctagtgttcgggaagggcacaagactgtctgttattgcaaacattcaaaaccccgaccctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 10 - DNA, 3300 bp, synthetic construct
  source 1..3300: mol_type = other DNA
atggcattgcctgttacagctctgctgctgcccctggctctgcttctgcatgctgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtctctggtcatcaccctgtactgcaaccaccggaacaggcggagagtgtgcaagtgccctagacctgtggtcaagagcggcgacaagcctagcctgagcgccagatatgttggcagcggagaaggcagaggcagcctgcttacatgcggcgacgtggaagagaaccccggacctatgaggcctagactgtggctgcttctggctgcccagctgacagtgctgcacggcaattctgtcctgcagcagacccctgcctacatcaaggtgcagaccaacaagatggtcatgctgagctgcgaggccaagatcagcctgtccaacatgcggatctactggctgcggcagagacaggcccctagcagcgattctcaccacgagtttctggccctgtgggatagcgccaagggaaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaacgcgtgtgcagactgcccaggcctgagacacaaaagggccctctgtgcagccctatcacactgggattgctggtggctggcgttctggtcctgctggtttctctgggagttgccatccacctgtgctgcagacgcagaagggccagactgcggttcatgaagcagttctacaaaggcagcggcgccaccaacttcagcctgctgaaacaagccggcgacgtcgaagaaaatcctggaccaatgggcagcagactgctgtgctgggttctgctgtgtctgcttggagccggacctgtgaaagctggcgtgacccagacacctagatacctgatcaagaccagaggccagcaagtgacactgagctgtagccccatcagcggccacagaagcgtgtcctggtatcagcagactcctggacagggcctgcagttcctgttcgagtacttctccgagacacagaggaacaagggcaacttccccggcagattctccggcagacagttcagcaactcccgcagcgagatgaacgtgtccacactggaactgggagatagcgccctgtacctgtgtgcctcttctgtgggaaccgtggaacagtacttcggccctggcacaagactgaccgtgaccgaggacctgaagaacgtgttcccacctgaggtggccgtgttcgagccttctgaggccgagatctctcacacccagaaagccacactcgtgtgtctggccaccggcttctatcccgatcacgtggaactgtcttggtgggtcaacggcaaagaggtgcacagcggcgtctgtaccgatcctcagccactgaaagagcagcccgctctgaacgacagcagatactgcctgtcctccagactgagagtgtccgccaccttctggcagaaccccagaaaccacttcaggtgtcaggtgcagttttacggcctgagcgagaacgacgagtggacccaggatagagccaagcctgtgacacagatcgtgtctgccgaagcctggggcagagccgattgtggctttaccagcgagagctaccagcagggcgttctgtctgccaccatcctgtacgagatcctgctgggcaaagccactctgtacgccgtgttggtgtctgccctggtgctgatggccatggtcaagcggaaggattctagaggcggatccggagccacaaatttctcactgctgaagcaggccggggatgttgaggaaaacccaggacctatggctatgctgctgggagcctctgtgctgatcctgtggctgcaacccgattgggtcaacagccagcagaagaacgacgaccagcaagtcaagcagaacagccccagcctgagcgtgcaagagggcagaatcagcatcctgaactgcgactacaccaactctatgttcgactactttctgtggtacaagaagtaccccgccgagggacccaccttcctgatcagcatcagcagcatcaaggacaagaacgaggacggccggttcaccgtgtttctgaacaagagcgccaagcacctgagcctgcacatcgtgccttctcagcctggcgatagcgccgtgtacttttgtgctgccagcggcgtgtcaggcaacacccctctggtttttggcaagggcacacgcctgtccgtgatcgccaacattcagaaccctgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagtccgatttcgcctgcgccaacgccttcaacaacagcattatccccgaggacacattcttcccaagtcctgagtccagctgcgacgtgaagctggtggaaaagagcttcgagacagacaccaacctgaacttccagaatctgagcgtgatcggcttcagaatcctgctgctgaaggtggccggattcaacctgctgatgaccctcagactgtggtccagctga

SEQ ID NO: 11 - protein, 280 aa, unidentified
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 12 - protein, 259 aa, unidentified
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - protein, 118 aa, synthetic construct
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIA

SEQ ID NO: 14 - protein, 6 aa, synthetic construct
NSMFDY

SEQ ID NO: 15 - protein, 7 aa, synthetic construct
ISSIKDK

SEQ ID NO: 16 - protein, 14 aa, synthetic construct
CAASGVSGNTPLVF

SEQ ID NO: 17 - protein, 12 aa, synthetic construct
AASGVSGNTPLV

SEQ ID NO: 18 - protein, 141 aa, unidentified
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - protein, 141 aa, synthetic construct
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 259 aa, synthetic construct
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 21 - protein, 309 aa, unidentified
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 22 - protein, 288 aa, unidentified
GVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 109 aa, synthetic construct
GVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVT

SEQ ID NO: 24 - protein, 5 aa, synthetic construct
SGHRS

SEQ ID NO: 25 - protein, 6 aa, synthetic construct
YFSETQ

SEQ ID NO: 26 - protein, 12 aa, synthetic construct
CASSVGTVEQYF

SEQ ID NO: 27 - protein, 10 aa, synthetic construct
ASSVGTVEQY

SEQ ID NO: 28 - protein, 179 aa, unidentified
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 29 - protein, 179 aa, synthetic construct
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 30 - protein, 288 aa, synthetic construct
GVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 611 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 32 - protein, 1099 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGSGEGRGSLLTCGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKGSGATNFSLLKQAGDVEENPGPMGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 33 - DNA, 825 bp, unidentified
  source 1..825: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggaccctatgaacaccaatgcaggcaaatcaacctttggggatgggactacgctcactgtgaagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 34 - DNA, 945 bp, unidentified
  source 1..945: mol_type = other DNA
atgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagcccctacggggggagcgtctcctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 35 - DNA, 1833 bp, synthetic construct
  source 1..1833: mol_type = other DNA
atgggcacaagacttctctgttgggctgcactgtgcttgcttggagctgagctgacagaagctggagttgcccaatctcctaggtacaagatcatcgagaagcggcagtctgtggccttttggtgcaatcccattagcggacatgccaccctgtactggtatcagcaaattctgggacagggccctaaactgctgatccagttccagaataacggcgtggtggacgattctcaactgcctaaggaccggttttctgccgagagactgaaaggcgttgatagcaccctgaagatccaacctgccaaactggaggattctgccgtgtacctgtgtgctagcagcccttatggaggatctgtgtcttatgagcagtacttcggacctggcaccagactgaccgtgactgaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggagacactgcttggcgtatcactggtgattctgtggctgcaactggctagagtgaactctcagcagggagaagaggatcctcaagctctgagcattcaggaaggcgaaaacgcaaccatgaattgctcatacaagaccagcatcaacaacctgcagtggtaccggcagaatagcggaagaggactggttcacctgattttaatcaggtctaatgaaagggagaagcacagcggcagactgagagttaccctggacacatccaagaaatcttcttctctgctgattacagcctctagagccgccgatacagccagctacttttgtgccacagatcccatgaacaccaatgccggaaagagcacattcggcgatggcacaaccctgacagttaagcccaatatccagaatcccgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 36 - DNA, 3297 bp, synthetic construct
  source 1..3297: mol_type = other DNA
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggcagcggagaaggcagaggctccctgcttacatgcggcgacgtggaagagaaccccggacctatgaggcctagactgtggctgcttctggctgcccagctgacagtgctgcacggcaattctgtcctgcagcagacccctgcctacatcaaggtgcagaccaacaagatggtcatgctgagctgcgaggccaagatcagcctgtccaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaacgcgtgtgcagactgcccaggcctgagacacaaaagggccctctgtgcagccctatcacactgggattgctggtggctggcgttctggtcctgctggtgtctctgggagttgccatccacctgtgctgtagaagaaggcgggccagactgcggttcatgaagcagttctacaaaggcagcggcgccaccaacttcagcctgctgaaacaagccggcgacgtcgaggaaaatcctggacctatgggcacaagacttctctgttgggctgcactgtgcttgcttggagctgagctgacagaagctggagttgcccaatctcctaggtacaagatcatcgagaagcggcagtctgtggccttttggtgcaatcccattagcggacatgccaccctgtactggtatcagcaaattctgggacagggccctaaactgctgatccagttccagaataacggcgtggtggacgattctcaactgcctaaggaccggttttctgccgagagactgaaaggcgttgatagcaccctgaagatccaacctgccaaactggaggattctgccgtgtacctgtgtgctagcagcccttatggaggatctgtgtcttatgagcagtacttcggacctggcaccagactgaccgtgactgaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccaccaacttcagcctgcttaaacaggccggcgacgtggaagagaaccctggacctatggagacactgcttggcgtatcactggtgattctgtggctgcaactggctagagtgaactctcagcagggagaagaggatcctcaagctctgagcattcaggaaggcgaaaacgcaaccatgaattgctcatacaagaccagcatcaacaacctgcagtggtaccggcagaatagcggaagaggactggttcacctgattttaatcaggtctaatgaaagggagaagcacagcggcagactgagagttaccctggacacatccaagaaatcttcttctctgctgattacagcctctagagccgccgatacagccagctacttttgtgccacagatcccatgaacaccaatgccggaaagagcacattcggcgatggcacaaccctgacagttaagcccaatatccagaatcccgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 37 - protein, 274 aa, unidentified
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 253 aa, unidentified
QQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 39 - protein, 112 aa, synthetic construct
QQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKP

SEQ ID NO: 40 - protein, 5 aa, synthetic construct
TSINN

SEQ ID NO: 41 - protein, 7 aa, synthetic construct
IRSNERE

SEQ ID NO: 42 - protein, 15 aa, synthetic construct
CATDPMNTNAGKSTF

SEQ ID NO: 43 - protein, 13 aa, synthetic construct
ATDPMNTNAGKST

SEQ ID NO: 44 - protein, 141 aa, unidentified; identical to SEQ ID NO: 18

SEQ ID NO: 45 - protein, 141 aa, synthetic construct; identical to SEQ ID NO: 19

SEQ ID NO: 46 - protein, 253 aa, synthetic construct
QQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 47 - protein, 314 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 48 - protein, 293 aa, synthetic construct
GVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 49 - protein, 114 aa, synthetic construct
GVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVT

SEQ ID NO: 50 - protein, 5 aa, synthetic construct
SGHAT

SEQ ID NO: 51 - protein, 6 aa, synthetic construct
FQNNGV

SEQ ID NO: 52 - protein, 16 aa, synthetic construct
CASSPYGGSVSYEQYF

SEQ ID NO: 53 - protein, 14 aa, synthetic construct
ASSPYGGSVSYEQY

SEQ ID NO: 54 - protein, 179 aa, unidentified; identical to SEQ ID NO: 28

SEQ ID NO: 55 - protein, 179 aa, synthetic construct; identical to SEQ ID NO: 29

SEQ ID NO: 56 - protein, 293 aa, synthetic construct
GVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 57 - protein, 610 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 1098 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGSGEGRGSLLTCGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKGSGATNFSLLKQAGDVEENPGPMGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 59 - protein, 132 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLADIYEQYFGPGTRLTVT

SEQ ID NO: 60 - protein, 132 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDRQSSGDKLTFGTGTRLAVRP

SEQ ID NO: 61 - protein, 113 aa, synthetic construct
GEDVEQSLFLSVREGDSSVINCTYTDSSSTYLYWYKQEPGAGLQLLTYIFSNMDMKQDQRLTVLLNKKDKHLSLRIADTQTGDSAIYFCAEPIIGGNTPLVFGKGTRLSVIAN

SEQ ID NO: 62 - protein, 119 aa, synthetic construct
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSSEGLAGGPTAGELFFGEGSRLTVL

SEQ ID NO: 63 - protein, 114 aa, synthetic construct
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGASGTYKYIFGTGTRLKVLAN

SEQ ID NO: 64 - protein, 113 aa, synthetic construct
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLALSYEQYFGPGTRLTVT

SEQ ID NO: 65 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 66 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 67-68 - skipped (2 entries)

SEQ ID NO: 69 - protein, 141 aa, synthetic construct
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 70 - protein, 177 aa, unidentified
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 71 - protein, 177 aa, synthetic construct
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 72 - protein, 179 aa, unidentified
EDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - protein, 179 aa, synthetic construct
EDLKNVFPPKVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 74 - protein, 22 aa, unidentified
GSGATNFSLLKQAGDVEENPGP

SEQ ID NO: 75 - protein, 24 aa, unidentified
LEGGGEGRGSLLTCGDVEENPGPR

SEQ ID NO: 76 - protein, 20 aa, Equine rhinitis A virus
QCTNYALLKLAGDVESNPGP

SEQ ID NO: 77 - protein, 25 aa, unidentified
GSGVKQTLNFDLLKLAGDVESNPGP

SEQ ID NO: 78 - protein, 189 aa, unidentified
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 79 - protein, 189 aa, unidentified
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 80 - protein, 232 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRGRKKLLYIFKQPFMRPVQTTQEEDGCSCRFPEEEEGGCEL

SEQ ID NO: 81 - protein, 173 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 82 - protein, 56 aa, synthetic construct
LCLLLLPIPLIVWVKRGRKKLLYIFKQPFMRPVQTTQEEDGCSCRFPEEEEGGCEL

SEQ ID NO: 83 - protein, 696 aa, synthetic construct
ATGCTGGGCATCTGGACCCTGCTGCCCTTGGTGCTGACTAGCGTGGCTAGACTGAGCAGCAAGAGCGTGAACGCCCAAGTGACCGACATCAACAGCAAGGGCCTGGAGCTGAGAAAGACCGTGACCACCGTGGAGACACAGAACCTGGAGGGCCTGCACCACGACGGGCAGTTCTGCCACAAGCCCTGCCCCCCCGGCGAGAGAAAGGCTAGAGACTGCACCGTGAACGGCGACGAGCCCGACTGCGTGCCCTGCCAAGAGGGCAAGGAGTACACCGACAAGGCCCACTTCAGCAGCAAGTGCAGAAGATGCAGACTGTGCGACGAGGGCCACGGCCTGGAGGTGGAGATCAACTGCACGCGTACGCAGAATACCAAATGCCGCTGCAAGCCCAACTTCTTCTGCAACAGCACCGTGTGCGAGCACTGCGACCCCTGCACCAAGTGCGAGCACGGCATCATCAAGGAGTGCACCCTGACAAGCAACACCAAGTGTAAGGAAGAGGGCTCACGGAGCAACCTGGGCTGGCTGTGCCTGCTGCTGCTGCCCATCCCCCTGATCGTGTGGGTGAAGAGAGGCAGAAAGAAGCTGCTGTACATCTTCAAGCAGCCCTTCATGAGACCCGTGCAGACCACCCAAGAGGAGGACGGGTGCAGCTGTAGATTCCCCGAAGAAGAAGAAGGCGGCTGTGAGCTT

SEQ ID NO: 84 - protein, 9 aa, synthetic construct
LVVVGAAGV

SEQ ID NO: 85 - protein, 9 aa, synthetic construct
LLVVGAAGV

SEQ ID NO: 86 - protein, 9 aa, synthetic construct
LMVVGAAGV

SEQ ID NO: 87 - protein, 9 aa, synthetic construct
LVVVGACGV

SEQ ID NO: 88 - protein, 9 aa, synthetic construct
LLVVGACGV

SEQ ID NO: 89 - protein, 9 aa, synthetic construct
LMVVGACGV

SEQ ID NO: 90 - protein, 9 aa, synthetic construct
LVVVGAVGV

SEQ ID NO: 91 - protein, 9 aa, synthetic construct
LLVVGAVGV

SEQ ID NO: 92 - protein, 9 aa, synthetic construct
LMVVGAVGV

SEQ ID NO: 93 - protein, 9 aa, synthetic construct
GLKDLLNPI

SEQ ID NO: 94 - protein, 9 aa, synthetic construct
GLKDLLNPV

SEQ ID NO: 95 - protein, 9 aa, synthetic construct
GMKDLLNPV

SEQ ID NO: 96 - protein, 9 aa, synthetic construct
ILNREIDFA

SEQ ID NO: 97 - protein, 9 aa, synthetic construct
ILNREIDFV

SEQ ID NO: 98 - protein, 9 aa, synthetic construct
IMNREIDFV

SEQ ID NO: 99 - protein, 9 aa, synthetic construct
ILNREIDFL

SEQ ID NO: 100 - protein, 65 aa, synthetic construct
HAGLSNRLARDNELRENDKEQLKAISTRDPLSEITEQEKDFLWSHRHYCVTIPEILPKLLLSVKW

SEQ ID NO: 101 - protein, 25 aa, synthetic construct
HAGLSNRLARDNELRENDKEQLKAI

SEQ ID NO: 102 - protein, 65 aa, synthetic construct
LSNRLARDNELRENDKEQLKAISTRDPLSEITEQEKDFLWSHRHYCVTIPEILPKLLLSVKWNSR

SEQ ID NO: 103 - protein, 65 aa, synthetic construct
LSNRLARDNELRENDKEQLKAISTRDPLSEITKQEKDFLWSHRHYCVTIPEILPKLLLSVKWNSR

SEQ ID NO: 104 - protein, 43 aa, synthetic construct
KTLALDKTEQEALEYFMKQMNDAHHGGWTTKMDWIFHTIKQHA

SEQ ID NO: 105 - protein, 43 aa, synthetic construct
KTLALDKTEQEALEYFMKQMNDARHGGWTTKMDWIFHTIKQHA

SEQ ID NO: 106 - protein, 43 aa, synthetic construct
KTLALDKTEQEALEYFMKQMNDALHGGWTTKMDWIFHTIKQHA

SEQ ID NO: 107 - protein, 6 aa, synthetic construct
DSAIYN

SEQ ID NO: 108 - protein, 7 aa, synthetic construct
IQSSQRE

SEQ ID NO: 109 - protein, 13 aa, synthetic construct
CAVKGSDDYKLSF

SEQ ID NO: 110 - protein, 5 aa, synthetic construct
KGHSH

SEQ ID NO: 111 - protein, 5 aa, synthetic construct
KGHSH

SEQ ID NO: 112 - protein, 19 aa, synthetic construct
CASSPVNLAGVSRADTQYF

SEQ ID NO: 113 - protein, 131 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVKGSDDYKLSFGAGTTVTVRA

SEQ ID NO: 114 - protein, 268 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVKGSDDYKLSFGAGTTVTVRANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 115 - protein, 138 aa, synthetic construct
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPVNLAGVSRADTQYFGPGTRLTVL

SEQ ID NO: 116 - protein, 311 aa, synthetic construct
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPVNLAGVSRADTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 117 - protein, 6 aa, synthetic construct
TSGFNG

SEQ ID NO: 118 - protein, 6 aa, synthetic construct
NVLDGL

SEQ ID NO: 119 - protein, 11 aa, synthetic construct
CAVTSWGKLQF

SEQ ID NO: 120 - protein, 5 aa, synthetic construct
SGHTA

SEQ ID NO: 121 - protein, 6 aa, synthetic construct
FQGNSA

SEQ ID NO: 122 - protein, 13 aa, synthetic construct
CASSPRGYQPQHF

SEQ ID NO: 123 - protein, 129 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVTSWGKLQFKLQFGAGTQVVVTP

SEQ ID NO: 124 - protein, 266 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVTSWGKLQFKLQFGAGTQVVVTPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 125 - protein, 132 aa, synthetic construct
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSPRGYQPQHFGDGTRLSIL

SEQ ID NO: 126 - protein, 305 aa, synthetic construct
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSPRGYQPQHFGDGTRLSILEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 127 - protein, 7 aa, synthetic construct
TSDQSYG

SEQ ID NO: 128 - protein, 8 aa, synthetic construct
QGSYDEQN

SEQ ID NO: 129 - protein, 15 aa, synthetic construct
CAMREVLDNTDKLIF

SEQ ID NO: 130 - protein, 5 aa, synthetic construct
KGHSH

SEQ ID NO: 131 - protein, 6 aa, synthetic construct
LQKENI

SEQ ID NO: 132 - protein, 16 aa, synthetic construct
CASSPPEAGLDTEAFF

SEQ ID NO: 133 - protein, 136 aa, synthetic construct
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREVLDNTDKLIFGTGTRLQVFP

SEQ ID NO: 134 - protein, 273 aa, synthetic construct
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREVLDNTDKLIFGTGTRLQVFPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 135 - protein, 135 aa, synthetic construct
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPPEAGLDTEAFFGQGTRLTVV

SEQ ID NO: 136 - protein, 308 aa, synthetic construct
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPPEAGLDTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 137 - protein, 6 aa, synthetic construct
NSAFQY

SEQ ID NO: 138 - protein, 6 aa, synthetic construct
TYSSGN

SEQ ID NO: 139 - protein, 13 aa, synthetic construct
CAMNSGGYQKVTF

SEQ ID NO: 140 - protein, 6 aa, synthetic construct
DFQATT

SEQ ID NO: 141 - protein, 7 aa, synthetic construct
SNEGSKA

SEQ ID NO: 142 - protein, 14 aa, synthetic construct
CSAREQGPLEEQYF

SEQ ID NO: 143 - protein, 133 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMNSGGYQKVTFGIGTKLQVIP

SEQ ID NO: 144 - protein, 268 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMNSGGYQKVTFIFTKLQVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNNFQNLLVIVLRILLLKVAGFNLLMTLRLWSS

SEQ ID NO: 145 - protein, 134 aa, synthetic construct
MLLLLLLLGPAGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSACSAREQGPLEEQYFGPGTRLTVT

SEQ ID NO: 146 - protein, 307 aa, synthetic construct
MLLLLLLLGPAGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSACSAREQGPLEEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 147 - protein, 7 aa, synthetic construct
TISGNEY

SEQ ID NO: 148 - protein, 5 aa, synthetic construct
GLKNN

SEQ ID NO: 149 - protein, 14 aa, synthetic construct
CIVRVAGSARQLTF

SEQ ID NO: 150 - protein, 5 aa, synthetic construct
SGHDT

SEQ ID NO: 151 - protein, 6 aa, synthetic construct
YYEEEE

SEQ ID NO: 152 - protein, 14 aa, synthetic construct
CASSFGTATYEQYF

SEQ ID NO: 153 - protein, 128 aa, synthetic construct
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVAGSARQLTFGSGTQLTVLP

SEQ ID NO: 154 - protein, 265 aa, synthetic construct
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVAGSARQLTFGSGTQLTVLPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 155 - protein, 132 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSFGTATYEQYFGPGTRLTVT

SEQ ID NO: 156 - protein, 305 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSFGTATYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 157 - protein, 6 aa, synthetic construct
DSASNY

SEQ ID NO: 158 - protein, 7 aa, synthetic construct
IRSNVGE

SEQ ID NO: 159 - protein, 14 aa, synthetic construct
CAASIPGTASKLTF

SEQ ID NO: 160 - protein, 5 aa, synthetic construct
MNHEY

SEQ ID NO: 161 - protein, 6 aa, synthetic construct
SMNVEV

SEQ ID NO: 162 - protein, 15 aa, synthetic construct
CASSPYRQGSYGYTF

SEQ ID NO: 163 - protein, 132 aa, synthetic construct
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASIPGTASKLTFGTGTRLQVTL

SEQ ID NO: 164 - protein, 269 aa, synthetic construct
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASIPGTASKLTFGTGTRLQVTLNIQNPEPAVYQLKDPRSQDSTLCLFTDFDSQINVPKTMESGTFITDKCVLDMKAMDSKSNGAIAWSNQTSFTCQDIFKETNATYPSSDVPCSATLTEKSFETDMNLNFQNLLVIVLRILLLKVAGFNLLMTLRLWSS

SEQ ID NO: 165 - protein, 133 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPYRQGSYGYTFGSGTRLTVV

SEQ ID NO: 166 - protein, 306 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPYRQGSYGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 167 - protein, 6 aa, synthetic construct
NTAFDY

SEQ ID NO: 168 - protein, 7 aa, synthetic construct
IRPDVSE

SEQ ID NO: 169 - protein, 13 aa, synthetic construct
CAASTGNFNKFYF

SEQ ID NO: 170 - protein, 5 aa, synthetic construct
SGHNS

SEQ ID NO: 171 - protein, 6 aa, synthetic construct
FNNNVP

SEQ ID NO: 172 - protein, 14 aa, synthetic construct
CASNRQGTVTEAFF

SEQ ID NO: 173 - protein, 140 aa, synthetic construct
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASTGNFNKFYFGSGTKLNVKP

SEQ ID NO: 174 - protein, 277 aa, synthetic construct
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASTGNFNKFYFGSGTKLNVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 175 - protein, 133 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASNRQGTVTEAFFGQGTRLTVV

SEQ ID NO: 176 - protein, 305 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASNRQGTVTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRHFRCQVQFHGLSEEDKWPEGSPKPVTQNISAEAWGRADCGITSASYQQGVLSATILYEILLGKATLYAVLVSTLVVMAMVKRKNS

SEQ ID NO: 177 - protein, 9 aa, synthetic construct
KTYPVQLWV

SEQ ID NO: 178 - protein, 9 aa, synthetic construct
KLYPVQLWV

SEQ ID NO: 179 - protein, 9 aa, synthetic construct
KMYPVQLWV

SEQ ID NO: 180 - protein, 9 aa, synthetic construct
KMYPVQLWL

SEQ ID NO: 181 - protein, 9 aa, synthetic construct
KLYPVQLWI

SEQ ID NO: 182 - protein, 9 aa, synthetic construct
GLAPPQLLI

SEQ ID NO: 183 - protein, 9 aa, synthetic construct
GLAPPQLLV

SEQ ID NO: 184 - protein, 9 aa, synthetic construct
GMAPPQLLV

SEQ ID NO: 185 - protein, 9 aa, synthetic construct
GLAPPQYLI

SEQ ID NO: 186 - protein, 9 aa, synthetic construct
GLAPPQYLV

SEQ ID NO: 187 - protein, 9 aa, synthetic construct
GMAPPQYLV

SEQ ID NO: 188 - protein, 9 aa, synthetic construct
ALNNMFCQL

SEQ ID NO: 189 - protein, 9 aa, synthetic construct
ALNNMFCQV

SEQ ID NO: 190 - protein, 10 aa, synthetic construct
ANINNMFCQV

SEQ ID NO: 191 - protein, 9 aa, synthetic construct
NMFCQLAKT

SEQ ID NO: 192 - protein, 9 aa, synthetic construct
NLFCQLAKV

SEQ ID NO: 193 - protein, 9 aa, synthetic construct
QLWVDSTPL

SEQ ID NO: 194 - protein, 9 aa, synthetic construct
QLWVDSTPI

SEQ ID NO: 195 - protein, 9 aa, synthetic construct
QLWVDSTPV

SEQ ID NO: 196 - protein, 9 aa, synthetic construct
RLILTIITL

SEQ ID NO: 197 - protein, 9 aa, synthetic construct
RLILTIITV

SEQ ID NO: 198 - protein, 9 aa, synthetic construct
YQGSYGFLL

SEQ ID NO: 199 - protein, 9 aa, synthetic construct
YQGSYGFLI

SEQ ID NO: 200 - protein, 9 aa, synthetic construct
YQGSYGFLV

SEQ ID NO: 201 - protein, 9 aa, synthetic construct
SVTCTYFPA

SEQ ID NO: 202 - protein, 9 aa, synthetic construct
SMTCTYFPL

SEQ ID NO: 203 - protein, 9 aa, synthetic construct
SLTCTYFPV

SEQ ID NO: 204 - protein, 9 aa, synthetic construct
SMTCTYFPV

SEQ ID NO: 205 - protein, 9 aa, synthetic construct
SLTCTYFPL

SEQ ID NO: 206 - protein, 9 aa, synthetic construct
SMTCTYFPI

SEQ ID NO: 207 - protein, 9 aa, synthetic construct
LLGRNSFEM

SEQ ID NO: 208 - protein, 9 aa, synthetic construct
LLGRNSFEL

SEQ ID NO: 209 - protein, 9 aa, synthetic construct
LLGRNSFEI

SEQ ID NO: 210 - protein, 9 aa, synthetic construct
LLGRNSFEV

SEQ ID NO: 211 - protein, 9 aa, synthetic construct
VVPCEPPEV

SEQ ID NO: 212 - protein, 9 aa, synthetic construct
VLPCEPPEV

SEQ ID NO: 213 - protein, 9 aa, synthetic construct
VMPCEPPEV

SEQ ID NO: 214 - protein, 142 aa, synthetic construct
METLLGLLILWLQLQWVSSKQEVTQIPAALSVPEGENLVLNCSFDSAIYNLQWFRQDPGKGLTSLLLIQSSQREQTSGRLNASLDKSSGRSTLYIAASQPGDSATYLCAPNDYKLSFGAGTTVTVRANIQNPDPAVYQLRDS

SEQ ID NO: 215 - protein, 146 aa, synthetic construct
MGCRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFSSDNEQFFGPGTRLTVLEDLKNVFPPEVAVFE

SEQ ID NO: 216 - protein, 145 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDSNDYKLSFGAGTTVTVRANIQNPDPAVYQLRDS

SEQ ID NO: 217 - protein, 145 aa, synthetic construct
MDIWLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSWGGELFFGEGSRLTVLEDLKNVFPPEVAVFE

SEQ ID NO: 218 - protein, 153 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSDAGGTSYGKLTFGQGTILTVHPNIQNPDPAVYQLRDS

SEQ ID NO: 219 - protein, 149 aa, synthetic construct
MSIGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSPRTGGAGNTIYFGEGSWLTVVEDLNKVFPPEVAVFE

SEQ ID NO: 220 - protein, 151 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDIEVVNAGNMLTFGGGTRLMVKPHIQNPDPAVYQLRDS

SEQ ID NO: 221 - protein, 146 aa, synthetic construct
MDIWLVCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASTDRVRYEQFFGPGTRLTVLEDLKNVFPPEVAVFE

SEQ ID NO: 222 - protein, 150 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFLGSGNTGKLIFGQGTTLQVKPDIQNPDPAVYQLRDS

SEQ ID NO: 223 - protein, 146 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAIRPSGGAFEQYFGPGTRLTVTEDLKNVFPPEVAVFE

SEQ ID NO: 224 - protein, 147 aa, synthetic construct
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAARATSGTYKYIFGTGTRLKVLANIQNPDPAVYQLRDS

SEQ ID NO: 225 - protein, 145 aa, synthetic construct
MLLLLLLLGPGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSASFSRDIETQYFGPGTRLLVLEDLKNVFPPEVAVFE

SEQ ID NO: 226 - protein, 143 aa, synthetic construct
MWGAFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRLGDAGNMLTFGGGTRLMVKPHIQNPDPAVYQLRDS

SEQ ID NO: 227 - protein, 148 aa, synthetic construct
MGTRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGRGMNTEAFFGQGTRLTVVEDLNKVFPPEVAVFE

SEQ ID NO: 228 - protein, 142 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCADNAGNMLTFGGGTRLMVKPHIQNPDPAVYQLRDS

SEQ ID NO: 229 - protein, 146 aa, synthetic construct
MLLLLLLLGPAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDPGRNYGYTFGSGTRLTVVEDLNKVFPPEVAVFE

SEQ ID NO: 230 - protein, 142 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCGDNAGNMLTFGGGTRLMVKPHIQNPDPAVYQLRDS

SEQ ID NO: 231 - protein, 145 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDEDRPYGYTFGSGTRLTVVEDLNKVFPPEVAVFE

SEQ ID NO: 232 - protein, 148 aa, synthetic construct
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSDGGATNKLIFGTGTLLAVQPNIQNPDPAVYQLRDS

SEQ ID NO: 233 - protein, 148 aa, synthetic construct
MGTRLLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSGGRASFTQYFGPGTRLTVLEDLKNVFPPEVAVFE

SEQ ID NO: 234 - protein, 148 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSDPNNQGGKLIFGQGTELSVKPNIQNPDPAVYQLRDS

SEQ ID NO: 235 - protein, 147 aa, synthetic construct
MGTRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQVRGTTEAFFGQGTRLTVVEDLNKVFPPEVAVFE

SEQ ID NO: 236 - protein, 142 aa, synthetic construct
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAEPNTDKLIFGTGTRLQVFPNIQNPDPAVYQLRDS

SEQ ID NO: 237 - protein, 146 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDTAMYLCATNYDRGYEQYFGPGTRLTVTEDLKNVFPPEVAVFE

SEQ ID NO: 238 - protein, 146 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVWATGNQFYFGTGTSLTVIPNIQNPDPAVYQLRDS

SEQ ID NO: 239 - protein, 146 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDTAMYLCATSFDRGYEQYFGPGTRLTVTEDLKNVFPPEVAVFE

SEQ ID NO: 240 - protein, 147 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNERNSGYALNFGKGTSLLVTPHIQNPDPAVYQLRDS

SEQ ID NO: 241 - protein, 147 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGPDSNQPQHFGDGTRLSILEDLNKVFPPEVAVFE

SEQ ID NO: 242 - protein, 147 aa, synthetic construct
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGQLRYGGATNKLIFGTGTLLAVQPNIQNPDPAVYQLRDS

SEQ ID NO: 243 - protein, 147 aa, synthetic construct
MGCRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSVGGASYEQYFGPGTRLTVTEDLKNVFPPEVAVFE

SEQ ID NO: 244 - protein, 144 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGPTYNQGGKLIFGQGTELSVKPNIQNPDPAVYQLRDS

SEQ ID NO: 245 - protein, 143 aa, synthetic construct
MLLLLLLLGPGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSARTAEVELFFGSGTQLSVLEDLNKVFPPEVAVFE

SEQ ID NO: 246 - protein, 150 aa, synthetic construct
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAVASWGSGSARQLTFGSGTQLTVLPDIQNPDPAVYQLRDS

SEQ ID NO: 247 - protein, 146 aa, synthetic construct
MGTRLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLDAGYEQYFGPGTRLTVTEDLKNVFPPEVAVFE

SEQ ID NO: 248 - protein, 146 aa, synthetic construct
METVLQVLLGILGFQAAWVSSQELEQSPQSLIVQEGKNLTINCTSSKTLYGLYWYKQKYGEGLIFLMMLQKGGEEKSHEKITAKLDEKKQQSSLHITASQPSHAGIYLCGADMLDYQKVTFGIGTKLQVIPNIQNPDPAVYQLRDS

SEQ ID NO: 249 - protein, 146 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRRGGSEQYFGPGTRLTVTEDLKNVFPPEVAVFE

SEQ ID NO: 250 - protein, 151 aa, synthetic construct
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREGQDSSYKLIFGSGTRLLVRPDIQNPDPAVYQLRDS

SEQ ID NO: 251 - protein, 147 aa, synthetic construct
MGTRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVLGPNTEAFFGQGTRLTVVEDLNKVFPPEVAVFE

SEQ ID NO: 252 - protein, 152 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSNAGGTSYGKLTFGQGTILTVHPNIQNPDPAVYQLRDS

SEQ ID NO: 253 - protein, 148 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSRRLPPPYGYTFGSGTRLTVVEDLNKVFPPEVAVFE

SEQ ID NO: 254 - protein, 150 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESSNTGKLIFGQGTTLQVKPDIQNPDPAVYQLRDS

SEQ ID NO: 255 - protein, 148 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSRREPPPYGYTFGSGTRLTVVEDLNKVFPPEVAVFE

SEQ ID NO: 256 - protein, 151 aa, synthetic construct
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREIRDSSYKLIFGSGTRLLVRPDIQNPDPAVYQLRDS

SEQ ID NO: 257 - protein, 147 aa, synthetic construct
MGTRLLCCVAFCLLGAGPVDSGVTQTPKHLITATGQRVTLRCSPRSGDLSVYWYQQSLDQGLQFLIQHYNGEERAKGNILERFSAQQFPDLHSELNLSSLELGDSALYFCASSPAGPNTEAFFGQGTRLTVVEDLNKVFPPEVAVFE

SEQ ID NO: 258 - protein, 354 aa, synthetic construct
GAGGTGCAGCTGTTGGAGTCTGGGGGAGGCTTGGTACAGCCTGGGGGGTCCCTGAGACTCTCCTGTGCAGCCTCTGGATTCACCTTTAGCAGCTATGCCATGAGCTGGGTCCGCCAGGCTCCAGGGAAGGGGCTGGAGTGGGTCTCAGCTATTAGTGGTAGTGGTGGTAGCACATACTACGCAGACTCCGTGAAGGGCCGGTTCACCATCTCCAGAGACAATTCCAAGAACACGCTGTATCTGCAAATGAACAGCCTGAGAGCCGAGGACACGGCGGTGTACTACTGCGCAAGAGCCGAGATGGGAGCCGTATTCGACATATGGGGTCAGGGTACAATGGTCACCGTCTCCTCA

SEQ ID NO: 259 - DNA, 366 bp, synthetic construct
  source 1..366: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcgtatgatggaagtaataaatactatgcagactccgtgaagggccgattcaccatctccagagacaattccaagaacacgctgtatctgcaaatgaacagcctgagagccgaggacacggcggtgtactactgcgccagagacggtacttatctaggtggtctctggtacttcgacttatgggggagaggtaccttggtcaccgtctcctca

SEQ ID NO: 260 - DNA, 348 bp, synthetic construct
  source 1..348: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctggtggtggtagcacaagctacgcacagaagttccagggcagagtcaccatgaccagggacacgtccacgagcacagtctacatggagctgagcagcctgagatctgaggacacggcggtgtactactgcgccagagagagttggccaatggacgtatggggccagggaacaactgtcaccgtctcctca

SEQ ID NO: 261 - DNA, 366 bp, synthetic construct
  source 1..366: mol_type = other DNA
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagaggcaggggatatgcaaccagcttagccttcgatatctggggtcagggtacaatggtcaccgtctcctca

SEQ ID NO: 262 - DNA, 357 bp, synthetic construct
  source 1..357: mol_type = other DNA
gaggtgcagctggtggagtctgggggaggcttggtacagcctggggggtccctgagactctcctgtgcagcctctggattcaccttcagtagctatagcatgaactgggtccgccaggctccagggaaggggctggagtgggtttcaaccattagtagtagtagtagtaccatatactacgcagactctgtgaagggccgattcaccatctccagagacaatgccaagaactcactgtatctgcaaatgaacagcctgagagctgaggacacggcggtgtactactgcgccagaggttctcaggagcacctgattttcgattattggggacagggtacattggtcaccgtctcctca

SEQ ID NO: 263 - DNA, 366 bp, synthetic construct
  source 1..366: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcgtatgatggaagtaataaatactatgcagactccgtgaagggccgattcaccatctccagagacaattccaagaacacgctgtatctgcaaatgaacagcctgagagccgaggacacggcggtgtactactgcgccagaactgacttctggagcggatcccctccaggcttagattactggggacagggtacattggtcaccgtctcctca

SEQ ID NO: 264 - DNA, 378 bp, synthetic construct
  source 1..378: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagaactcctgaatactcctccagcatatggcactattactacggcatggacgtatggggccagggaacaactgtcaccgtctcctca

SEQ ID NO: 265 - DNA, 369 bp, synthetic construct
  source 1..369: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcgtatgatggaagtaataaatactatgcagactccgtgaagggccgattcaccatctccagagacaattccaagaacacgctgtatctgcaaatgaacagcctgagagccgaggacacggcggtgtactactgcgtcaaggggccgttgcaggagccgccatacgattatggaatggacgtatggggccagggaacaactgtcaccgtctcctca

SEQ ID NO: 266 - DNA, 321 bp, synthetic construct
  source 1..321: mol_type = other DNA
gaaattgtgttgacacagtctccagccaccctgtctttgtctccaggggaaagagccaccctctcctgcagggccagtcagagtgttagcaggtacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagagaatctcctggcctttcacttttggcggagggaccaaggttgagatcaaa

SEQ ID NO: 267 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcagggactcggcctccctctcacttttggcggagggaccaaggttgagatcaaa

SEQ ID NO: 268 - DNA, 318 bp, synthetic construct
  source 1..318: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtacgccgcctaccctacttttggcggagggaccaaggttgagatcaaa

SEQ ID NO: 269 - DNA, 321 bp, synthetic construct
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagagacacgtctggcctcctacttttggcggagggaccaaggttgagatcaaa

SEQ ID NO: 270 - DNA, 321 bp, synthetic construct
  source 1..321: mol_type = other DNA
gaaattgtgttgacacagtctccagccaccctgtctttgtctccaggggaaagagccaccctctcctgcagggccagtcagagtgttagcaggtacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagagattctactacccttggacttttggcggagggaccaaggttgagatcaaa

SEQ ID NO: 271 - DNA, 321 bp, synthetic construct
  source 1..321: mol_type = other DNA
gacatccagttgacccagtctccatcttccgtgtctgcatctgtaggagacagagtcaccatcacttgtcgggcgagtcagggtattagcagctggttagcctggtatcagcagaaaccagggaaagcccctaagctcctgatctatggtgcatccagtttgcaaagtggggtcccatcaaggttcagcggcagtggatctgggacagatttcactctcaccatcagcagcctgcagcctgaagattttgcaacttattactgtcagcagatatacaccttccctttcacttttggcggagggaccaaggttgagatcaaa

SEQ ID NO: 272 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagttcgcccacactcctttcacttttggcggagggaccaaggttgagatcaaa

SEQ ID NO: 273 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatagcgcatccaccagggccactggtatcccagccaggttcagtggcagtgggtctgggacagagttcactctcaccatcagcagcctgcagtctgaagattttgcagtttattactgtcagcagcaccacgtctggcctctcacttttggcggagggaccaaggttgagatcaaa

SEQ ID NO: 274 - protein, 118 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 275 - protein, 122 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 276 - protein, 116 aa, synthetic construct
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 277 - protein, 122 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 278 - protein, 119 aa, synthetic construct
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 279 - protein, 122 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 280 - protein, 126 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 281 - protein, 123 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 282 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 283 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 284 - protein, 106 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 285 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 286 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 287 - protein, 107 aa, synthetic construct
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 288 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 289 - protein, 107 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 290 - DNA, 366 bp, synthetic construct
  source 1..366: mol_type = other DNA
caggtgcagctgcaggaatccggaccggggctggtgaagcccagcgagactctgagtctcacgtgtacagtttctggaggtagcattagctcctactattggtcatggataaggcagccccccgggaagggattggaatggatcggctatatttactacagtgggagcaccaattacaacccctcactgaagtctagagttacaatcagcgttgacacctcaaagaatcagttcagtttgaaattgtctagcgtcacagcagctgatacagccgtctattattgtgtttctctggtctattgcggtggggattgttacagtggctttgactattgggggcagggtactctggttacagtttcttcc

SEQ ID NO: 291 - DNA, 372 bp, synthetic construct
  source 1..372: mol_type = other DNA
caggtacagctgcaggaatctgggcccggacttgtcaagccaagtcagacactttctcttacatgtaccgtgagcggcggaagtataagcagtggaggcttttactggtcttggatacggcagcacccaggcaaaggcttggagtggattggatacattcatcattcaggatctacacactataatccatcccttaagtcccgggtcaccattagcattgatacgtctaagaatctgttcagtctcaggctgtcctccgtcactgctgccgacacagccgtgtactactgcgcctccttggtttactgcggaggcgactgttatagcggctttgattattgggggcaggggaccctcgtaaccgtgagctct

SEQ ID NO: 292 - DNA, 363 bp, synthetic construct
  source 1..363: mol_type = other DNA
caggtccaactggtgcagtccggagccgaagtcaagaaaccaggtgcctccgttaaagtgagttgcaaagtctctggatacactctgaccgagctctctatgcactgggtccggcaggcccccggcaagggattggaatggatgggcgggttcgatcctgaggacggagagactatctacgctcaaaaattccagggacgagtgactgtgaccgaagacactagtaccgacactgcctacatggaactttcctctctgcgatcagaagataccgcagtgtactactgtgctactgaatctaggggcattggatggccctacttcgattactggggtcagggaactctggtgactgtctccagc

SEQ ID NO: 293 - DNA, 351 bp, synthetic construct
  source 1..351: mol_type = other DNA
caggtccagttggtcgaaagtggcggtggtgtagtgcagccgggccgcagtttgaggctttcctgtgcggcttcaggctttactttttccagctatggaatgcactgggtgcggcaggcccccggcaaaggacttgagtgggtggccgtcatttcttatgacggatcagataagtactacgtggacagcgtcaagggcagattcaccatctctagggacaacagtaaaaatagactctacctccagatgaatagcctcagagctgaagacacggccgtctactattgtgctcgggagcggtatagtggcagagactactgggggcagggcacactcgttacagtgagtagc

SEQ ID NO: 294 - DNA, 324 bp, synthetic construct
  source 1..324: mol_type = other DNA
gacatccagttgacacagagcccgagttccttgtccgcctccgtcggggatagagtgtcatttacctgtcaggcctctcaggatattaataactttctgaattggtatcagcaaaagcccggaaaggcacccaagctgttgatttacgacgccagtaacctggagacaggcgtgccctcccggtttagtggtagcggaagcggtacggattttacctttactatcagctctctccaacccgaagacattgcaacctactattgtcaacaatatggaaacctgccttttacatttggcggcggcaccaaggtggagattaagcgg

SEQ ID NO: 295 - DNA, 324 bp, synthetic construct
  source 1..324: mol_type = other DNA
gatatccagctcactcaaagcccctctagtctctctgcctcagtgggggatcgggtcagttttacttgtcaagcttcacaggatatcaacaacttccttaattggtatcagcagaagccaggaaaagcacccaagctgctcatctatgatgcctcaaatttggagacgggtgttcccagtcgattctctgggtcagggtccgggaccgactttacgtttacgatctcctctctgcagcccgaagacatcgccacatactattgtcaacagtacggcaacttgcctttcacatttgggggcgggactaaggttgaaatcaagagg

SEQ ID NO: 296 - DNA, 324 bp, synthetic construct
  source 1..324: mol_type = other DNA
gatattcagatgactcaatctccttcttctctgtccgcttccgtgggcgatagagtgaccattacttgtagggcgtcccagtcaatctccagttatttgaattggtatcagcagaagcccgggaaagcacctaagctgttgatcagcggggcttctagcctgaagagtggggtaccttcacggttcagcggaagcggaagcggaaccgatttcaccctgactatcagcagcctgccacctgaggactttgcaacttactactgccaacagtcatacagcactccgatcactttcggccagggcacccggctcgaaatcaagcgc

SEQ ID NO: 297 - DNA, 324 bp, synthetic construct
  source 1..324: mol_type = other DNA
gagattgttatgacccagagtcctgcgaccctctcagtcagccccggggagcgcgcaactttgtcttgcagagctagtcagtccgtgtcctctcttctgacatggtaccagcaaaagcccgggcaggctccgcgccttttgatctttggggcttcaacaagagccactgggattcccgcacgattctctggctccgggagcggtactggtttcaccctgacgattagcagtctccagagcgaggacttcgccgtatactactgccagcagtacgatacgtggccattcacttttggaccagggactaaagtggattttaagcgc

SEQ ID NO: 298 - protein, 122 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSS

SEQ ID NO: 299 - protein, 124 aa, synthetic construct
QVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSS

SEQ ID NO: 300 - protein, 121 aa, synthetic construct
QVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSS

SEQ ID NO: 301 - protein, 117 aa, synthetic construct
QVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSS

SEQ ID NO: 302 - protein, 108 aa, synthetic construct
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKR

SEQ ID NO: 303 - protein, 108 aa, synthetic construct; identical to SEQ ID NO: 302

SEQ ID NO: 304 - protein, 108 aa, synthetic construct
DIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKR

SEQ ID NO: 305 - protein, 108 aa, synthetic construct
EIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKR

SEQ ID NO: 306 - DNA, 36 bp, synthetic construct
  source 1..36: mol_type = other DNA
ggaattccatatgagtcaacaaggagaagaagatcc

SEQ ID NO: 307 - DNA, 36 bp, synthetic construct
  source 1..36: mol_type = other DNA
ttgtcagtcgacttagagtctctcagctggtacacg

SEQ ID NO: 308 - DNA, 36 bp, synthetic construct
  source 1..36: mol_type = other DNA
tctctcatatggatggtggaattactcaatccccaa

SEQ ID NO: 309 - DNA, 32 bp, synthetic construct
  source 1..32: mol_type = other DNA
tagaaaccggtggccaggcacaccagtgtggc

SEQ ID NO: 310 - protein, 274 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQGENSGYSTLTFGKGTMLLVSPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 311 - protein, 21 aa, synthetic construct
MEKMLECAFIVLWLQLGWLSG

SEQ ID NO: 312 - protein, 112 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQ

SEQ ID NO: 313 - protein, 6 aa, synthetic construct
VSGLRG

SEQ ID NO: 314 - skipped

SEQ ID NO: 315 - protein, 15 aa, synthetic construct
CAVQGENSGYSTLTF

SEQ ID NO: 316 - protein, 19 aa, synthetic construct
NSGYSTLTFGKGTMLLVSP

SEQ ID NO: 317 - protein, 141 aa, synthetic construct
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 318 - protein, 314 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGPGLAAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 319 - protein, 133 aa, synthetic construct
MGPQLLGYVVLCLLGAGPLMGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSL

SEQ ID NO: 320 - protein, 5 aa, synthetic construct
MNHEY

SEQ ID NO: 321 - protein, 6 aa, synthetic construct
SMNVEV

SEQ ID NO: 322 - protein, 16 aa, synthetic construct
CASSLGPGLAAYNEQF

SEQ ID NO: 323 - protein, 15 aa, synthetic construct
YNEQFFGPGTRLTVL

SEQ ID NO: 324 - protein, 179 aa, synthetic construct; identical to SEQ ID NO: 28

SEQ ID NO: 325 - protein, 272 aa, synthetic construct
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEYSSASKIIFGSGTRLSIRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 326 - protein, 21 aa, synthetic construct
MKTFAGFSFLFLWLQLDCMSR

SEQ ID NO: 327 - protein, 112 aa, synthetic construct
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAE

SEQ ID NO: 328 - protein, 6 aa, synthetic construct
DSSSTY

SEQ ID NO: 329 - skipped

SEQ ID NO: 330 - protein, 12 aa, synthetic construct
CAEYSSASKIIF

SEQ ID NO: 331 - protein, 19 aa, synthetic construct
YSSASKIIFGSGTRLSIRP

SEQ ID NO: 332 - protein, 141 aa, synthetic construct; identical to SEQ ID NO: 18

SEQ ID NO: 333 - protein, 310 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRANTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 334 - protein, 19 aa, synthetic construct
MGSWTLCCVSLCILVAKHT

SEQ ID NO: 335 - protein, 113 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCAS

SEQ ID NO: 336 - protein, 5 aa, synthetic construct
SGHDY

SEQ ID NO: 337 - protein, 6 aa, synthetic construct
FNNNVP

SEQ ID NO: 338 - protein, 12 aa, synthetic construct
CASRANTGELFF

SEQ ID NO: 339 - protein, 16 aa, synthetic construct
NTGELFFGEGSRLTVL

SEQ ID NO: 340 - protein, 179 aa, synthetic construct; identical to SEQ ID NO: 28

SEQ ID NO: 341 - protein, 277 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEGNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 342 - protein, 20 aa, synthetic construct
MLTASLLRAVIASICVVSSM

SEQ ID NO: 343 - protein, 116 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSE

SEQ ID NO: 344 - protein, 7 aa, synthetic construct
TRDTTYY

SEQ ID NO: 345 - protein, 4 aa, synthetic construct
RNSF

SEQ ID NO: 346 - protein, 15 aa, synthetic construct
CALSEGNSGNTPLVF

SEQ ID NO: 347 - protein, 19 aa, synthetic construct
NSGNTPLVFGKGTRLSVIA

SEQ ID NO: 348 - protein, 141 aa, synthetic construct; identical to SEQ ID NO: 18

SEQ ID NO: 349 - protein, 313 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLSSGSHQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 350 - protein, 19 aa, synthetic construct
MGIRLLCRVAFCFLAVGLV

SEQ ID NO: 351 - protein, 114 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSL

SEQ ID NO: 352 - protein, 5 aa, synthetic construct
MDHEN

SEQ ID NO: 353 - protein, 6 aa, synthetic construct
SYDVKM

SEQ ID NO: 354 - protein, 16 aa, synthetic construct
CASSLSSGSHQETQYF

SEQ ID NO: 355 - protein, 15 aa, synthetic construct
QETQYFGPGTRLLVL

SEQ ID NO: 356 - protein, 179 aa, synthetic construct; identical to SEQ ID NO: 28

SEQ ID NO: 357 - DNA, 423 bp, synthetic construct
  source 1..423: mol_type = other DNA
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 358 - DNA, 420 bp, synthetic construct
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 359 - DNA, 420 bp, synthetic construct
  source 1..420: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagtgcgtgctggacatgcggagcatggacttcaagagcaactccgccgtggcctggtccaacaagtccgatttcgcctgcgccaacgccttcaacaacagcattatccccgaggacacattcttcccaagcagcgacgtgccctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 360 - DNA, 420 bp, synthetic construct
  source 1..420: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 361 - DNA, 411 bp, synthetic construct
  source 1..411: mol_type = other DNA
cctgatcctgccgtgtaccagctgcgggatagcaagagcagcgacaagagcgtgtgcctgttcaccgacttcgacagccagaccaacgtgtctcagtctaaggatagtgatgtgtatatcaccgacaagaccgtgctggacatgcggagcatggacttcaagagcaacagcgccgtggcctggtccaacaagagcgacttcgcctgcgccaacgccttcaacaacagcatcatccccgaggacaccttcttccccagccccgagagcagctgcgacgtgaaactggtggagaagagcttcgagacagacaccaacctgaacttccagaacctgagcgtgatcggcttcagaatcctgctgctgaaggtggccggcttcaacctgctgatgaccctgcggctgtggagcagc

SEQ ID NO: 362 - DNA, 420 bp, synthetic construct
  source 1..420: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagtgcgtgctggacatgcggagcatggacttcaagagcaactccgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 363 - DNA, 411 bp, synthetic construct
  source 1..411: mol_type = other DNA
cctgatcctgccgtgtaccagctgcgggatagcaagagcagcgacaagagcgtgtgtctgttcaccgacttcgacagccagaccaacgtgtcccagagcaaggactccgacgtgtacatcaccgataagtgcgtgctggacatgcggagcatggacttcaagagcaactccgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 364 - DNA, 420 bp, synthetic construct
  source 1..420: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagcagcgacgtgccctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 365 - DNA, 420 bp, synthetic construct
  source 1..420: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaaccgcgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 366 - DNA, 420 bp, synthetic construct
  source 1..420: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaaccgcgccgtggcctggtccaacaagtccgatttcgcctgcgccaacgccttcaacaacagcattatccccgaggacacattcttcccaagcagcgacgtgccctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 367 - DNA, 420 bp, synthetic construct
  source 1..420: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagcagcgacagcagctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 368 - DNA, 717 bp, synthetic construct
  source 1..717: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcggtacccagagttttgggttgttggaccctaaattgtgctatctgttggacggaatcctgttcatttacggagtgatcctgaccgcattgttcctgcgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 369 - DNA, 408 bp, synthetic construct
  source 1..408: mol_type = other DNA
atccagaaccctgaacctgccgtgtaccagctgaaggaccccagaagccaggacagcaccctgtgcctgttcaccgacttcgacagccagatcaacgtgcccaagacaatggaaagcggcaccttcatcaccgacaagaccgtgctggacatgaaggctatggacagcaagagcaacggcgccattgcctggtccaaccagaccagcttcacatgccaggacatcttcaaagagacaaacgccacctacccctccagcgacgtgccctgtgacgccaccctgaccgagaagtccttcgagacagacatgaacctcaacttccagaacctgagcgtgatgggcctgcgaatcctgctgctgaaagtggccggcttcaacctgctgatgaccctgcggctgtggtccagc

SEQ ID NO: 370 - DNA, 399 bp, synthetic construct
  source 1..399: mol_type = other DNA
gtgcaggaccctgaccctagcgtgtaccagatgagaagccccaagaccggcaggaccgtgtgcctgttcaccgacttcgacagccaggccgacttccagaagcctgagggggccaccgtgatctccctgaacagcaccgtgctggacatgaaagtgatggacagcaagagcaacggcgctctgacctggaacagcgagacaaacatcgagtgcaaagagaacttccagcagcccttctaccccagcagcaactactcctgcgacgccaagctgatcgagaagtccttcgagacagacatggacctgaacttctacaacctgctggtcatcggcctgagaatcctgctgctgaaagtgatcggcttcaacctgttcatgaccctgaggctgtggtcctgc

SEQ ID NO: 371 - DNA, 531 bp, synthetic construct
  source 1..531: mol_type = other DNA
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 372 - DNA, 537 bp, synthetic construct
  source 1..537: mol_type = other DNA
gaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 373 - DNA, 537 bp, synthetic construct; identical to SEQ ID NO: 372
  source 1..537: mol_type = other DNA

SEQ ID NO: 374 - DNA, 537 bp, synthetic construct
  source 1..537: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtgtaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 375 - DNA, 537 bp, synthetic construct
  source 1..537: mol_type = other DNA
gaggacctgaagaacgtgttcccacctgaggtggccgtgttcgagcctagcaaggccgagatcgcccacacccagaaagccaccctcgtgtgcctggctaccggcttctaccccgaccatgtggaactgtcttggtgggtcaacggcaaagaggtgcacagcggcgtgtgtaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggcatcaccagcgccagctaccaccagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 376 - DNA, 537 bp, synthetic construct
  source 1..537: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 377 - DNA, 536 bp, synthetic construct
  source 1..536: mol_type = other DNA
gaccggtgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 378 - DNA, 531 bp, synthetic construct
  source 1..531: mol_type = other DNA
gaggacctgaagaacgtgttcccacctgaggtggccgtgttcgagcccagcgaggccgagatttcacacactcagaaagccacactcgtatgtctggcgactggtttctttcccgaccacgtggagctgtcttggtgggtgaacggcaaagaggtgcacagcggggtctctaccgacccccagccactgaaagagcagcccgccctgaacgacagccggtactgcctctcttctcggctgagagtgtccgccaccttctggcagaacccccggaaccacttcagatgccaggtgcagttctacggcctgagcgagaacgacgagtggacccaggacagagccaagcctgtgacccagatcgtgagtgctgaagcgtggggacgcgccgattgcggctttaccagcgagagctaccagcagggcgtgctgtctgccaccatcctgtacgagatcctgctgggcaaggccaccctgtacgccgtgctggtgtccgccctggtgctgatggctatggtgaagcggaaggacttc

SEQ ID NO: 379 - DNA, 531 bp, synthetic construct
  source 1..531: mol_type = other DNA
gaggacctgaacaaggtgttcccacctgaggtggccgtgttcgagcccagcgaggccgagatttcacacactcagaaagccacactcgtatgtctggcgactggtttctttcccgaccacgtggagctgtcttggtgggtgaacggcaaagaggtgcacagcggggtctctaccgacccccagccactgaaagagcagcccgccctgaacgacagccggtactgcctctcttctcggctgagagtgtccgccaccttctggcagaacccccggaaccacttcagatgccaggtgcagttctacggcctgagcgagaacgacgagtggacccaggacagagccaagcctgtgacccagatcgtgagtgctgaagcgtggggacgcgccgattgcggctttaccagcgtgagctaccagcagggcgtgctgtctgccaccatcctgtacgagatcctgctgggcaaggccaccctgtacgccgtgctggtgtccgccctggtgctgatggctatggtgaagcggaaggacttc

SEQ ID NO: 380 - DNA, 537 bp, synthetic construct
  source 1..537: mol_type = other DNA
gaggacctgaagaacgtgttcccacctgaggtggccgtgttcgagcctagcaaggccgagatcgcccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggcatcaccagcgccagctaccaccagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 381 - DNA, 537 bp, synthetic construct
  source 1..537: mol_type = other DNA
gaggacctgaagaacgtgttcccacctgaggtggccgtgttcgagcctagcaaggccgagatcgcccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 382 - DNA, 537 bp, synthetic construct
  source 1..537: mol_type = other DNA
gaggacctgaagaacgtgttcccacctgaggtggccgtgttcgagcctagcaaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggcatcaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 383 - DNA, 537 bp, synthetic construct
  source 1..537: mol_type = other DNA
gaggacctgaagaacgtgttcccacctgaggtggccgtgttcgagcctagcaaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgccagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 384 - DNA, 537 bp, synthetic construct
  source 1..537: mol_type = other DNA
gaggacctgaagaacgtgttcccacctgaggtggccgtgttcgagcctagcaaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccaccagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 385 - DNA, 536 bp, synthetic construct
  source 1..536: mol_type = other DNA
gaggacctgaagaacgtgttcccacctgaggtggccgtgttcgagcctagcagggccgagatcgcccacacccagaaagccaccctcgtgtgcctggctaccggcttctacccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 386 - DNA, 537 bp, synthetic construct
  source 1..537: mol_type = other DNA
gaggacctgaagaacgtgttcccacctgaggtggccgtgttcgagcctagcagggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 387 - DNA, 537 bp, synthetic construct
  source 1..537: mol_type = other DNA
gaggacctgaagaacgtgttcccacctgaggtggccgtgttcgagcctagcaaggccgagatcgcccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcggactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggcatcaccagcgccagctaccaccagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 388 - DNA, 537 bp, synthetic construct
  source 1..537: mol_type = other DNA
gaggacctgaagaacgtgttcccacctgaggtggccgtgttcgagcctagcaaggccgagatcgcccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcggactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 389 - DNA, 537 bp, synthetic construct
  source 1..537: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcggactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 390 - DNA, 513 bp, synthetic construct
  source 1..513: mol_type = other DNA
gaggatctgcggaacgtgaccccacccaaggtgtccctgttcgagcccagcaaggccgagatcgccaataaacagaaggccaccctcgtgtgcctggccagaggcttcttccccgaccacgtggaactgtcttggtgggtcaacggcaaagaggtgcacagcggcgtcagcaccgaccctcaggcctacaaagagagcaactacagctactgcctgagcagtcggctgcgggtgtccgccaccttctggcacaacccccggaaccacttcagatgccaggtgcagttccacggcctgagcgaagaggacaagtggcccgagggcagccccaagcctgtcacccagaacatcagcgccgaggcctggggcagagcctgtggcatcaccagcagctaccagcagggcgtgctgagcgccaccatcctgtacgagatcctgctgggcaaggccaccctgtacgccgtgctggtgtccaccctggtggtcatggctatggtcaagcggaagaacagc

SEQ ID NO: 391 - DNA, 531 bp, synthetic construct
  source 1..531: mol_type = other DNA
gaggacctccagaaagtgaccccccctaccgtgaccgtgttcgagccttctgaggccgagatcagccggacccagaaagccacactcgtgtgcctggctcggggcttctaccccgatcacgtggaactgtcttggtgggtcaacggcaaagaagtgaccagcggcttcagcaccgacccccagcctgacaaagagcggcccagcgagaacgacagcagctactgcctgagcagcagactgagagtgtccgccagcttctggcacaacccccggaaccacttcagatgccaggtgcagttctacggcctgggcgacgacgacgagtggaagtacgacagagtgaagcccatcacccagaacatcagcgccgaagcctggggcagagccgattgcggctttacctccgtcagctaccaccagggcgtgctgagcgccaccatcctgtacgagatcctgctgggcaaggccaccctgtacgccgtgctggtgtctatcctggtgctgatggctaaagtgaagcggaagggcagc

SEQ ID NO: 392 - DNA, 828 bp, synthetic construct
  source 1..828: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 393 - DNA, 807 bp, synthetic construct
  source 1..807: mol_type = other DNA
gggtgtccagccctacccacaggtgtgggcggcacaccctttccttctctggccccaccaatcatgctgctggtggatggaaagcagcagatggtggtggtctgcctggtccttgatgttgcaccccctggccttgacagccccatctggttctcagccggcaatggcagtgcactggatgccttcacctatggcccttccccagcaacggatggcacctggaccaacttggcccatctctccctgccttctgaggagctggcatcctgggagcctttggtctgccacactgggcctggggctgagggtcacagcaggagtacacagcccatgcatctgtcaggagaggcttctacagccaggacctgcccccaggagcctctcagggggacaccgggtggggcgctgtggctgggggtcctgcggctgctgctcttcaagctgctgctgtttgacctgctcctgacctgcagctgcctgtgcgaccccgcgggcccgctgccttcccccgcaaccaccacccgcctgcgagccctcggntcccatcgactgcacccggccacggagactgggggacgagaggccaccagctcacccagaccccagcctcgggaccgccgctggggtgacacccctccgggtcggaagcccgggagcccagtatggggggaagggtcttacctcagcagttaccccacttgcccagcacaggcctggtgctcaagatctcgcctcagggctccttcctccagtcttggagcattttttgcaggtgacctgcctcctcctctgcaggctggagctgcc

SEQ ID NO: 394 - DNA, 774 bp, synthetic construct
  source 1..774: mol_type = other DNA
accccattcccttctctggcccctcccatcatgctgctggtggacggcaagcagcagatggtggtcgtgtgcctggtgctggatgtggccccacctggactggacagccccatctggtttagcgccggcaatggctctgccctggacgcctttacctacggcccaagccctgccaccgatggcacctggacaaatctggcccacctgagcctgcccagcgaggaactggcttcttgggagcctctcgtgtgccacacaggacctggcgctgagggacacagcagatccacccagcctatgcacctgtctggcgaggccagcaccgccagaacctgtcctcaggaacctctgagaggcacccctggcggagcactgtggctgggagtgctgagactgctgctgttcaagctgctgctgtttgacctgctgctgacctgctcctgtctgtgcgatcctgccggccctctgccttctcctgccaccaccacaagactgagagccctgggcagccacagactgcaccctgccacagagacaggcggcagagaggccacaagcagccctagaccccagcccagagacagaagatggggcgataccccccctggcagaaagcctggatctccagtgtggggcgagggcagctacctgagcagctaccctacatgccctgcccaggcttggtgcagcagaagcgcactgagagcacccagctctagcctgggagccttctttgccggcgatctgcctccaccactccaggctggcgctgct

SEQ ID NO: 395 - DNA, 630 bp, synthetic construct
  source 1..630: mol_type = other DNA
accccattcccttctctggcccctcccatcatgctgctggtggacggcaagcagcagatggtggtcgtgtgcctggtgctggatgtggccccacctggactggacagccccatctggtttagcgccggcaatggctctgccctggacgcctttacctacggcccaagccctgccaccgatggcacctggacaaatctggcccacctgagcctgcccagcgaggaactggcttcttgggagcctctcgtgtgccacacaggacctggcgctgagggacacagcagatccacccagcctatgcacctgtctggcgaggccagcaccgccagaacctgtcctcaggaacctctgagaggcacccctggcggagcactgtggctgggagtgctgagactgctgctgttcaagctgctgctgtttgacctgctgctgacctgctcctgtctgtgcgatcctgccggccctctgccttctcctgccaccaccacaagactgagagccctgggcagccacagactgcaccctgccacagagacaggcggcagagaggccacaagcagccctagaccccagcccagagacagaagatggggcgataccccccctggcagaaagcctggatctccagtc

SEQ ID NO: 396 - DNA, 567 bp, synthetic construct
  source 1..567: mol_type = other DNA
gataaacaacttgatgcagatgtttcccccaagcccactatttttcttccttcaattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcaccacagtggatcccaaatacaattattcaaaggatgcaaatgatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatca

SEQ ID NO: 397 - DNA, 519 bp, synthetic construct
  source 1..519: mol_type = other DNA
gacaagcagctggacgccgacgtgtcccccaagcctaccatcttcctgccctctatcgccgagacaaagctccagaaggccggcacctacctgtgcctgctggaaaagttcttcccagatgtgattaagatccactggcaggaaaagaagtccaacaccatcctgggcagccaggaaggcaacaccatgaagaccaacgacacctacatgaagttcagctggctgaccgtgcccgagaagtccctggacaaagaacacagatgcatcgtgcggcacgaaaacaacaagaacggcgtggaccaggaaatcatcttcccacccatcaagaccgatgtgattacaatggaccccaaggacaactgctccaaggacgccaacgataccctgctgctccagctgaccaacaccagcgcctactacatgtacttgttgctgctgctgaagtccgtggtgtacttcgccatcatcacatgctgcctgctgcggagaaccgccttctgctgcaacggcgagaagtct

SEQ ID NO: 398 - DNA, 459 bp, synthetic construct
  source 1..459: mol_type = other DNA
agtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttg

SEQ ID NO: 399 - DNA, 459 bp, synthetic construct
  source 1..459: mol_type = other DNA
agccagcctcacaccaagcccagcgtgttcgtgatgaagaacggcaccaacgtggcctgcctcgtgaaagagttctaccccaaggacatcaggatcaacctggtgtccagcaagaagatcaccgagttcgaccccgccatcgtgatctcccccagcggcaagtacaacgccgtgaagctggggaagtacgaggacagcaacagcgtgacctgctccgtgcagcacgacaacaagaccgtgcacagcaccgactttgaagtgaaaaccgactccaccgaccacgtgaagcccaaagagacagagaacaccaagcagcccagcaagagctgccacaagcccaaggccatcgtgcacaccgagaaagtgaacatgatgagcctgaccgtgctgggcctgcggatgctgttcgccaaaaccgtggccgtgaacttcctgctgaccgccaagctgttcttcctc

SEQ ID NO: 400 - DNA, 321 bp, synthetic construct
  source 1..321: mol_type = other DNA
atccagaaccctgatcctgccgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 401 - DNA, 536 bp, synthetic construct
  source 1..536: mol_type = other DNA
gaggacctgaagaacgtgttcccacctgaggtggccgtgttcgagcctagcagggccgagatcagccacacccagaaagccaccctcgtgtgcctggctaccggcttctaccccgaccatgtggaactgtcttggtgggtcaacggcaaagaggtgcacagcggcgtgtgtaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcggagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 402 - DNA, 537 bp, synthetic construct
  source 1..537: mol_type = other DNA
gaggacctgaagaacgtgttcccacctgaggtggccgtgttcgagcctagcagggccgagatcgcccacacccagaaagccaccctcgtgtgcctggctaccggcttctaccccgaccatgtggaactgtcttggtgggtcaacggcaaagaggtgcacagcggcgtgtgtaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 403 - protein, 141 aa, synthetic construct; identical to SEQ ID NO: 317

SEQ ID NO: 404 - protein, 140 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 405 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 406 - protein, 137 aa, synthetic construct
PDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 407 - protein, 140 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 408 - protein, 137 aa, synthetic construct
PDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 409 - protein, 140 aa, synthetic construct
IQNPDPAVYQLRDSKSSDKSVCLFTDFDSQTNVSQSKDSDVYITDKTVLDMRSMDFKSNRAVAWSNKSDFACANAFNNSIIPEDTFFPSPESSCDVKLVEKSFETDTNLNFQNLSVIGFRILLLKVAGFNLLMTLRLWSS

SEQ ID NO: 410 - protein, 140 aa, synthetic construct
IQNPDPAVYQLRDSKSSDKSVCLFTDFDSQTNVSQSKDSDVYITDKTVLDMRSMDFKSNRAVAWSNKSDFACANAFNNSIIPEDTFFPSSDVPCDVKLVEKSFETDTNLNFQNLSVIGFRILLLKVAGFNLLMTLRLWSS

SEQ ID NO: 411 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSSDSSCDVKLVEKSFETDTNLNFQNLSVIGFRILLLKVAGFNLLMTLRLWSS

SEQ ID NO: 412 - protein, 239 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGTQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 413 - protein, 136 aa, synthetic construct
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 414 - protein, 133 aa, synthetic construct
VQDPDPSVYQMRSPKTGRTVCLFTDFDSQADFQKPEGATVISLNSTVLDMKVMDSKSNGALTWNSETNIECKENFQQPFYPSSNYSCDAKLIEKSFETDMDLNFYNLLVIGLRILLLKVIGFNLFMTLRLWSC

SEQ ID NO: 415 - protein, 177 aa, synthetic construct; identical to SEQ ID NO: 70

SEQ ID NO: 416 - protein, 179 aa, synthetic construct; identical to SEQ ID NO: 28

SEQ ID NO: 417 - protein, 179 aa, synthetic construct; identical to SEQ ID NO: 28

SEQ ID NO: 418 - protein, 179 aa, synthetic construct; identical to SEQ ID NO: 29

SEQ ID NO: 419 - protein, 179 aa, synthetic construct
EDLKNVFPPEVAVFEPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 420 - protein, 178 aa, synthetic construct
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 421 - protein, 177 aa, synthetic construct
EDLKNVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDF

SEQ ID NO: 422 - protein, 177 aa, synthetic construct; identical to SEQ ID NO: 70

SEQ ID NO: 423 - protein, 179 aa, synthetic construct
EDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 424 - protein, 179 aa, synthetic construct
EDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 425 - protein, 179 aa, synthetic construct
EDLKNVFPPEVAVFEPSKAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSESYQQGVLSATILYEILLGKATLYAVLVSALVLMAMVKRKDSRG

SEQ ID NO: 426 - protein, 179 aa, synthetic construct
EDLKNVFPPEVAVFEPSKAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSASYQQGVLSATILYEILLGKATLYAVLVSALVLMAMVKRKDSRG

SEQ ID NO: 427 - protein, 179 aa, synthetic construct
EDLKNVFPPEVAVFEPSKAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYHQGVLSATILYEILLGKATLYAVLVSALVLMAMVKRKDSRG

SEQ ID NO: 428 - protein, 179 aa, synthetic construct
EDLKNVFPPEVAVFEPSRAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 429 - protein, 179 aa, synthetic construct
EDLKNVFPPEVAVFEPSRAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 430 - protein, 179 aa, synthetic construct
EDLKNVFPPEVAVFEPSKAEIAHTQKATLVCLATGFYPDHVELSWWVNGKEVHSGVSTDPQPLKEQPALNDSRYCLSSGLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 431 - protein, 179 aa, synthetic construct
EDLKNVFPPEVAVFEPSKAEIAHTQKATLVCLATGFYPDHVELSWWVNGKEVHSGVSTDPQPLKEQPALNDSRYCLSSGLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 432 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSGLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 433 - protein, 171 aa, synthetic construct
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRACGITSSYQQGVLSATILYEILLGKATLYAVLVSTLVVMAMVKRKNS

SEQ ID NO: 434 - protein, 177 aa, synthetic construct
EDLQKVTPPTVTVFEPSEAEISRTQKATLVCLARGFYPDHVELSWWVNGKEVTSGFSTDPQPDKERPSENDSSYCLSSRLRVSASFWHNPRNHFRCQVQFYGLGDDDEWKYDRVKPITQNISAEAWGRADCGFTSVSYHQGVLSATILYEILLGKATLYAVLVSILVLMAKVKRKGS

SEQ ID NO: 435 - protein, 276 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGSQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 436 - protein, 269 aa, synthetic construct
GCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSRLRAPSSSLGAFFAGDLPPPLQAGAA

SEQ ID NO: 437 - protein, 258 aa, synthetic construct
TPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGAA

SEQ ID NO: 438 - protein, 210 aa, synthetic construct
TPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPV

SEQ ID NO: 439 - protein, 189 aa, synthetic construct
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYMYLLLLLKSVVYFAIITCCLLGRTAFCCNGEKS

SEQ ID NO: 440 - protein, 173 aa, synthetic construct
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 441 - protein, 153 aa, synthetic construct
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 442 - protein, 153 aa, synthetic construct; identical to SEQ ID NO: 441

SEQ ID NO: 443 - protein, 107 aa, synthetic construct
IQNPDPAVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 444 - protein, 179 aa, synthetic construct
EDLKNVFPPEVAVFEPSRAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 445 - protein, 179 aa, synthetic construct
EDLKNVFPPEVAVFEPSRAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 446 - DNA, 420 bp, synthetic construct
  source 1..420: mol_type = other DNA
atccagaaccctgatcctgccgtgtaccagctgcgggattgcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 447 - DNA, 420 bp, synthetic construct
  source 1..420: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatctgcgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 448 - DNA, 537 bp, synthetic construct
  source 1..537: mol_type = other DNA
gaggacctgaagaacgtgttcccacctgaggtggccgtgttctgccctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 449 - DNA, 537 bp, synthetic construct
  source 1..537: mol_type = other DNA
gaggacctgaagaacgtgttcccacctgaggtggccgtgttcgagccttgcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 450 - DNA, 537 bp, synthetic construct
  source 1..537: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctacctgcccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 451 - DNA, 537 bp, synthetic construct
  source 1..537: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgtgcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 452 - DNA, 396 bp, synthetic construct
  source 1..396: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcgga

SEQ ID NO: 453 - DNA, 393 bp, synthetic construct
  source 1..393: mol_type = other DNA
gaggacctgaagaacgtgttcccacctgaggtggccgtgttcgagcctagcaaggccgagatcgcccacacccagaaagccaccctcgtgtgcctggctaccggcttctaccccgaccatgtggaactgtcttggtgggtcaacggcaaagaggtgcacagcggcgtgtgtaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgc

SEQ ID NO: 454 - DNA, 393 bp, synthetic construct
  source 1..393: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtgtaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgc

SEQ ID NO: 455 - DNA, 90 bp, synthetic construct
  source 1..90: mol_type = other DNA
cagagcttcggcctgctggaccccaagctgtgctacctgctggatggcatcctgttcatctacggcgtgatcctgaccgccctgttcctg

SEQ ID NO: 456 - DNA, 429 bp, synthetic construct
  source 1..429: mol_type = other DNA
cagagcttcggcctgctggaccccaagctgtgctacctgctggatggcatcctgttcatctacggcgtgatcctgaccgccctgttcctgagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 457 - DNA, 429 bp, synthetic construct; identical to SEQ ID NO: 456
  source 1..429: mol_type = other DNA

SEQ ID NO: 458 - DNA, 123 bp, synthetic construct
  source 1..123: mol_type = other DNA
aggagtaagaggagcaggctcctgcacagtgactacatgaacatgactccacgccgccccggacccacccgcaagcattaccagccctatgccccaccacgcgacttcgcagcctatcgctcc

SEQ ID NO: 459 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 460 - DNA, 165 bp, synthetic construct
  source 1..165: mol_type = other DNA
aagaaccggaaggccaaggccaagcctgtgacaagaggtgctggtgctggcggcagacagagaggccagaacaaagaaagacctcctcctgtgcctaatcctgactacgagcccatcagaaaaggccagcgggatctgtacagcggcctgaaccagcggcggatc

SEQ ID NO: 461 - DNA, 828 bp, synthetic construct; identical to SEQ ID NO: 392
  source 1..828: mol_type = other DNA

SEQ ID NO: 462 - DNA, 828 bp, synthetic construct
  source 1..828: mol_type = other DNA
gaggacctgaagaacgtgttcccacctgaggtggccgtgttcgagcctagcaaggccgagatcgcccacacccagaaagccaccctcgtgtgcctggctaccggcttctaccccgaccatgtggaactgtcttggtgggtcaacggcaaagaggtgcacagcggcgtgtgtaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 463 - DNA, 954 bp, synthetic construct
  source 1..954: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtgtaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 464 - DNA, 954 bp, synthetic construct
  source 1..954: mol_type = other DNA
gaggacctgaagaacgtgttcccacctgaggtggccgtgttctgccctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 465 - DNA, 954 bp, synthetic construct
  source 1..954: mol_type = other DNA
gaggacctgaagaacgtgttcccacctgaggtggccgtgttcgagccttgcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 466 - DNA, 954 bp, synthetic construct
  source 1..954: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctacctgcccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 467 - DNA, 954 bp, synthetic construct
  source 1..954: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgtgcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 468 - DNA, 951 bp, synthetic construct
  source 1..951: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgaggagtaagaggagcaggctcctgcacagtgactacatgaacatgactccacgccgccccggacccacccgcaagcattaccagccctatgccccaccacgcgacttcgcagcctatcgctccagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 469 - DNA, 954 bp, synthetic construct
  source 1..954: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagaagcgccgacgcccctgcctaccagcagggacagaaccaactgtacaacgagctgaacctgggcagacgggaagagtacgacgtgctggacaagcggagaggcagggaccctgagatgggcggaaagccccagcggagaaagaacccccaggaaggactgtataacgaactccagaaagacaagatggccgaagcctacagcgagatcggcatgaagggcgagcggagaagaggcaagggccacgatggcctgtaccagggcctgagcaccgccaccaaggacacctatgacgccctgcacatgcaggccctgcctccaaga

SEQ ID NO: 470 - DNA, 780 bp, synthetic construct
  source 1..780: mol_type = other DNA
gaggacctgaagaacgtgttcccacctgaggtggccgtgttcgagcctagcgaggccgagatcagccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggatcccagagcttcggcctgctggaccccaagctgtgctacctgctggatggcatcctgttcatctacggcgtgatcctgaccgccctgttcctgaaacggggcagaaagaaactcctgtatatattcaaacaaccatttatgagaccagtacaaactactcaagaggaagatggctgtagctgccgatttccagaagaagaagaaggaggatgtgaactgaagaaccggaaggccaaggccaagcctgtgacaagaggtgctggtgctggcggcagacagagaggccagaacaaagaaagacctcctcctgtgcctaatcctgactacgagcccatcagaaaaggccagcgggatctgtacagcggcctgaaccagcggcggatc

SEQ ID NO: 471 - DNA, 282 bp, synthetic construct
  source 1..282: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgc

SEQ ID NO: 472 - DNA, 281 bp, synthetic construct
  source 1..281: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagtgcgtgctggacatgcggagcatggacttcaagagcaactccgccgtggcctggtccaacaagtccgatttcgcctgcgccaacgccttcaacaacagcattatccccgaggacacattcttcccaagcaggacgtgccctgc

SEQ ID NO: 473 - DNA, 282 bp, synthetic construct
  source 1..282: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagtgcgtgctggacatgcggagcatggacttcaagagcaactccgccgtggcctggtccaacaagtccgatttcgcctgcgccaacgccttcaacaacagcattatccccgaggacacattcttcccaagccccgagagcagctgc

SEQ ID NO: 474 - DNA, 90 bp, synthetic construct
  source 1..90: mol_type = other DNA
cagagttttgggttgttggaccctaaattgtgctatctgttggacggaatcctgttcatttacggagtgatcctgaccgcattgttcctg

SEQ ID NO: 475 - DNA, 339 bp, synthetic construct
  source 1..339: mol_type = other DNA
cgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 476 - DNA, 429 bp, synthetic construct
  source 1..429: mol_type = other DNA
cagagttttgggttgttggaccctaaattgtgctatctgttggacggaatcctgttcatttacggagtgatcctgaccgcattgttcctgcgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 477 - DNA, 123 bp, synthetic construct
  source 1..123: mol_type = other DNA
aggagcaagcggtcacggctgctccacagcgattacatgaatatgacaccacgaagaccgggtcctactcgcaaacactaccaaccctacgcacctccccgcgactttgcagcgtaccgatct

SEQ ID NO: 478 - DNA, 126 bp, synthetic construct
  source 1..126: mol_type = other DNA
aagagaggccggaagaagctgctgtacatcttcaagcagcctttcatgcggcccgtgcagaccacccaggaagaggacggctgctcctgccggttccccgaggaagaagaaggcggctgcgagctg

SEQ ID NO: 479 - DNA, 165 bp, synthetic construct
  source 1..165: mol_type = other DNA
aagaatagaaaggccaaagctaaacccgtgaccagaggcgctggcgcaggcggaaggcaaaggggacaaaacaaagaacggccaccaccagtgcaaaatccagattatgagcctattcggaaaggacagagggacctgtactctggcctgaatcagagaagaatc

SEQ ID NO: 480 - DNA, 717 bp, synthetic construct; identical to SEQ ID NO: 368
  source 1..717: mol_type = other DNA

SEQ ID NO: 481 - DNA, 717 bp, synthetic construct
  source 1..717: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagtgcgtgctggacatgcggagcatggacttcaagagcaactccgccgtggcctggtccaacaagtccgatttcgcctgcgccaacgccttcaacaacagcattatccccgaggacacattcttcccaagcagcgacgtgccctgcggtacccagagttttgggttgttggaccctaaattgtgctatctgttggacggaatcctgttcatttacggagtgatcctgaccgcattgttcctgcgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 482 - DNA, 717 bp, synthetic construct
  source 1..717: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagtgcgtgctggacatgcggagcatggacttcaagagcaactccgccgtggcctggtccaacaagtccgatttcgcctgcgccaacgccttcaacaacagcattatccccgaggacacattcttcccaagccccgagagcagctgcggtacccagagttttgggttgttggaccctaaattgtgctatctgttggacggaatcctgttcatttacggagtgatcctgaccgcattgttcctgcgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 483 - DNA, 717 bp, synthetic construct
  source 1..717: mol_type = other DNA
atccagaaccctgatcctgccgtgtgccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcggtacccagagttttgggttgttggaccctaaattgtgctatctgttggacggaatcctgttcatttacggagtgatcctgaccgcattgttcctgcgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 484 - DNA, 717 bp, synthetic construct
  source 1..717: mol_type = other DNA
atccagaaccctgatcctgccgtgtaccagctgcgggattgcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcggtacccagagttttgggttgttggaccctaaattgtgctatctgttggacggaatcctgttcatttacggagtgatcctgaccgcattgttcctgcgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 485 - DNA, 717 bp, synthetic construct
  source 1..717: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatctgcgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcggtacccagagttttgggttgttggaccctaaattgtgctatctgttggacggaatcctgttcatttacggagtgatcctgaccgcattgttcctgcgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 486 - DNA, 840 bp, synthetic construct
  source 1..840: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcggtacccagagttttgggttgttggaccctaaattgtgctatctgttggacggaatcctgttcatttacggagtgatcctgaccgcattgttcctgaggagcaagcggtcacggctgctccacagcgattacatgaatatgacaccacgaagaccgggtcctactcgcaaacactaccaaccctacgcacctccccgcgactttgcagcgtaccgatctcgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 487 - DNA, 843 bp, synthetic construct
  source 1..843: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcggtacccagagttttgggttgttggaccctaaattgtgctatctgttggacggaatcctgttcatttacggagtgatcctgaccgcattgttcctgaagagaggccggaagaagctgctgtacatcttcaagcagcctttcatgcggcccgtgcagaccacccaggaagaggacggctgctcctgccggttccccgaggaagaagaaggcggctgcgagctgcgggttaagttttccagatccgcagacgcacccgcttaccaacaagggcagaaccaactctacaatgaacttaatttggggcggagggaagagtacgacgttctggacaaaaggaggggacgagatcccgagatgggcggaaagcctcagcgaaggaagaatccgcaagaggggctttataacgaactccagaaagataaaatggcagaagcctatagtgagattgggatgaagggagaaaggaggaggggtaaaggtcacgatgggttgtaccaaggattgagcacagccactaaagatacctatgatgcgctccacatgcaggccctgccgccaaga

SEQ ID NO: 488 - DNA, 669 bp, synthetic construct
  source 1..669: mol_type = other DNA
atccagaaccctgatcctgccgtgtaccagctgcgggatagcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagccccgagagcagctgcggtacccagagttttgggttgttggaccctaaattgtgctatctgttggacggaatcctgttcatttacggagtgatcctgaccgcattgttcctgaagagaggccggaagaagctgctgtacatcttcaagcagcctttcatgcggcccgtgcagaccacccaggaagaggacggctgctcctgccggttccccgaggaagaagaaggcggctgcgagctgaagaatagaaaggccaaagctaaacccgtgaccagaggcgctggcgcaggcggaaggcaaaggggacaaaacaaagaacggccaccaccagtgcaaaatccagattatgagcctattcggaaaggacagagggacctgtactctggcctgaatcagagaagaatc

SEQ ID NO: 489 - protein, 140 aa, synthetic construct
IQNPDPAVCQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 490 - protein, 140 aa, synthetic construct
IQNPDPAVYQLRDCKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 491 - protein, 140 aa, synthetic construct
IQNPDPAVYQLRDSKSSDKSVCLFTDFDSQTNVSQSKDSDVYICDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 492 - protein, 179 aa, synthetic construct
EDLKNVFPPEVAVFCPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 493 - protein, 179 aa, synthetic construct
EDLKNVFPPEVAVFEPCEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 494 - protein, 179 aa, synthetic construct
EDLKNVFPPEVAVFEPSEAEISHTQKATLVCLATGFYPDHVELSWWVNGKEVHSGVSTCPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 495 - protein, 179 aa, synthetic construct
EDLKNVFPPEVAVFEPSEAEISHTQKATLVCLATGFYPDHVELSWWVNGKEVHSGVSTDPQPLKEQPALNDSRYCLCSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 496 - protein, 132 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCG

SEQ ID NO: 497 - protein, 131 aa, synthetic construct
EDLKNVFPPEVAVFEPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADC

SEQ ID NO: 498 - protein, 131 aa, synthetic construct
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADC

SEQ ID NO: 499 - protein, 30 aa, synthetic construct
QSFGLLDPKLCYLLDGILFIYGVILTALFL

SEQ ID NO: 500 - protein, 143 aa, synthetic construct
QSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 501 - protein, 143 aa, synthetic construct; identical to SEQ ID NO: 500

SEQ ID NO: 502 - protein, 41 aa, synthetic construct
RSKRSRLLHSDYMNMTPRRPGPTRKHYQPYAPPRDFAAYRS

SEQ ID NO: 503 - protein, 42 aa, synthetic construct
KRGRKKLLYIFKQPFMRPVQTTQEEDGCSCRFPEEEEGGCEL

SEQ ID NO: 504 - protein, 55 aa, synthetic construct
KNRKAKAKPVTRGAGAGGRQRGQNKERPPPVPNPDYEPIRKGQRDLYSGLNQRRI

SEQ ID NO: 505 - protein, 276 aa, synthetic construct; identical to SEQ ID NO: 435

SEQ ID NO: 506 - protein, 276 aa, synthetic construct
EDLKNVFPPEVAVFEPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGSQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 507 - protein, 302 aa, synthetic construct
EDLKNVFPPEVAVFEPATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGSQSFGLLDPKLCYLLDGILFIYGVILTALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 508 - protein, 318 aa, synthetic construct
EDLKNVFPPEVAVFCPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGSQSFGLLDPKLCYLLDGILFIYGVILTALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 509 - protein, 318 aa, synthetic construct
EDLKNVFPPEVAVFEPCEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGSQSFGLLDPKLCYLLDGILFIYGVILTALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 510 - protein, 318 aa, synthetic construct
EDLKNVFPPEVAVFEPSEAEISHTQKATLVCLATGFYPDHVELSWWVNGKEVHSGVSTCPQPLKEQPALNDSRYCLSSRLRVSATFWQNPRNHFRCQVQFYGLSENDEWTQDRAKPVTQIVSAEAWGRADCGSQSFGLLDPKLCYLLDGILFIYGVILTALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 511 - protein, 318 aa, synthetic construct
EDLKNVFPPEVAVFEPSEAEISHTQKATLVCLATGFYPDHVELSWWVNGKEVHSGVSTDPQPLKEQPALNDSRYCLCSRLRVSATFWQNPRNHFRCQVQFYGLSENDEWTQDRAKPVTQIVSAEAWGRADCGSQSFGLLDPKLCYLLDGILFIYGVILTALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 512 - protein, 317 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGSQSFGLLDPKLCYLLDGILFIYGVILTALFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 513 - protein, 318 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGSQSFGLLDPKLCYLLDGILFIYGVILTALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 514 - protein, 260 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGSQSFGLLDPKLCYLLDGILFIYGVILTALFLKRGRKKLLYIFKQPFMRPVQTTQEEDGCSCRFPEEEEGGCELKNRKAKAKPVTRGAGAGGRQRGQNKERPPPVPNPDYEPIRKGQRDLYSGLNQRRI

SEQ ID NO: 515 - protein, 239 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCGTQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 516 - protein, 239 aa, synthetic construct
IQNPDPAVCQLRDSKSSDKSVCLFTDFDSQTNVSQSKDSDVYITDKTVLDMRSMDFKSNSAVAWSNKSDFACANAFNNSIIPEDTFFPSPESSCGTQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 517 - protein, 239 aa, synthetic construct
IQNPDPAVYQLRDCKSSDKSVCLFTDFDSQTNVSQSKDSDVYITDKTVLDMRSMDFKSNSAVAWSNKSDFACANAFNNSIIPEDTFFPSPESSCGTQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 518 - protein, 239 aa, synthetic construct
IQNPDPAVYQLRDSKSSDKSVCLFTDFDSQTNVSQSKDSDVYICDKTVLDMRSMDFKSNSAVAWSNKSDFACANAFNNSIIPEDTFFPSPESSCGTQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 519 - protein, 280 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGTQSFGLLDPKLCYLLDGILFIYGVILTALFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 520 - protein, 281 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGTQSFGLLDPKLCYLLDGILFIYGVILTALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 521 - protein, 223 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGTQSFGLLDPKLCYLLDGILFIYGVILTALFLKRGRKKLLYIFKQPFMRPVQTTQEEDGCSCRFPEEEEGGCELKNRKAKAKPVTRGAGAGGRQRGQNKERPPPVQNPDYEPIRKGQRDLYSGLNQRRI

SEQ ID NO: 522 - protein, 140 aa, synthetic construct
IQNPDPAVYQLRDCKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 523 - protein, 140 aa, synthetic construct
IQNPDPAVYQLRDCKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 524 - protein, 179 aa, synthetic construct
EDLKNVFPPEVAVFCPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 525 - protein, 179 aa, synthetic construct
EDLKNVFPPEVAVFCPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 526 - DNA, 420 bp, synthetic construct
  source 1..420: mol_type = other DNA
atccagaaccctgatcctgccgtgtaccagctgcgggattgcaagagcagcgacaagagcgtgtgtctgttcaccgacttcgacagccagaccaacgtgtcccagagcaaggactccgacgtgtacatcaccgataagaccgtgctggacatgcggagcatggacttcaagagcaactccgccgtggcctggtccaacaagtccgatttcgcctgcgccaacgccttcaacaacagcattatccccgaggacacattcttcccaagcagcgacgtgccctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 527 - DNA, 420 bp, synthetic construct
  source 1..420: mol_type = other DNA
atccagaaccctgatcctgccgtgtaccagctgcgggattgcaagagcagcgacaagagcgtgtgtctgttcaccgacttcgacagccagaccaacgtgtcccagagcaaggactccgacgtgtacatcaccgataagtgcgtgctggacatgcggagcatggacttcaagagcaactccgccgtggcctggtccaacaagtccgatttcgcctgcgccaacgccttcaacaacagcattatccccgaggacacattcttcccaagcagcgacgtgccctgcgacgtgaagctggtggaaaagagcttcgagacagacaccaacctgaacttccagaacctgagcgtgatcggcttcagaatcctgctgctgaaagtggccggctttaacctgctgatgaccctgcggctgtggtccagc

SEQ ID NO: 528 - DNA, 537 bp, synthetic construct
  source 1..537: mol_type = other DNA
gaggacctgaagaacgtgttcccacctgaggtggccgtgttctgccctagcaaggccgagatcgcccacacccagaaagccaccctcgtgtgcctggctaccggcttctaccccgaccatgtggaactgtcttggtgggtcaacggcaaagaggtgcacagcggcgtgtctaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggcatcaccagcgccagctaccaccagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 529 - DNA, 537 bp, synthetic construct
  source 1..537: mol_type = other DNA
gaggacctgaagaacgtgttcccacctgaggtggccgtgttctgccctagcaaggccgagatcgcccacacccagaaagccaccctcgtgtgcctggctaccggcttctaccccgaccatgtggaactgtcttggtgggtcaacggcaaagaggtgcacagcggcgtgtgtaccgacccccagccactgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggcatcaccagcgccagctaccaccagggcgtgctgagcgccaccatcctgtacgagatcctgctgggcaaggccaccctgtacgccgtgctggtgtctgccctggtgctgatggctatggtcaagcggaaggacagccgggga

SEQ ID NO: 530 - DNA, 333 bp, synthetic construct
  source 1..333: mol_type = other DNA
cagtctgtgctgacacagcctcctagtgtgtctggcgctcctggacagagagtgaccatcagctgtacaggcagcagcagcaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactccaggccgaggacgaggccgactactactgtcagagctacgacagcagcctgagcggctctgtgtttggcacaggcacaaaggtgctggtgctt

SEQ ID NO: 531 - DNA, 336 bp, synthetic construct
  source 1..336: mol_type = other DNA
aacttcatgctgacccagcctcacagcgtgtccgagtctccaggcaagaccgtgaccatcagctgtacaggcagcagcggctctatcgccagcaactacgtgcagtggtatcagcagaggccaggcagcgcccctaccatcctgatctacgaggataacaagcggcctagcggcgtgcccgatagattttctggcagcatcgacagcagcagcaacagcgccagcctgacaatcagcggcctgaaaacaggcgacgaggccgactactactgccagagctacgacgacatcaaccactgggttttcggcggaggcaccaagctgacagttcttgga

SEQ ID NO: 532 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGTGTKVLVL

SEQ ID NO: 533 - protein, 113 aa, synthetic construct
NFMLTQPHSVSESPGKTVTISCTGSSGSIASNYVQWYQQRPGSAPTILIYEDNKRPSGVPDRFSGSIDSSSNSASLTISGLKTGDEADYYCQSYDDINHWVFGGGTKLTVLGQ

SEQ ID NO: 534 - protein, 9 aa, synthetic construct
SSNIGAGYD

SEQ ID NO: 535 - protein, 8 aa, synthetic construct
SGSIASNY

SEQ ID NO: 536 - protein, 4 aa, synthetic construct
GNSN

SEQ ID NO: 537 - protein, 4 aa, synthetic construct
EDNK

SEQ ID NO: 538 - protein, 11 aa, synthetic construct
QSYDSSLSGSV

SEQ ID NO: 539 - protein, 10 aa, synthetic construct
QSYDDINHWV

SEQ ID NO: 540 - DNA, 351 bp, synthetic construct
  source 1..351: mol_type = other DNA
caagttcagctggttcagagcggagccgaagtgaagaaacctggcagcagcgtgaaggtgtcctgcaaagcaagcggcggcaccttcagcagctacaccatcaacgtttggagacaggccccaggccagggccttgaatggatgggaggcttcatccctatcagcggcaccgtgaactacgcccagaaattccagggcagagtgacaatcaccgccgacgagagcacaagcaccgcctacatggaactgtccagcctgagaagcgaggacaccgccgtgtactactgtgccagacctctggattggaccgaggacatctggggccagggaacactggtcacagtgtctagt

SEQ ID NO: 541 - DNA, 357 bp, synthetic construct
  source 1..357: mol_type = other DNA
gaagttcagctggttcagagcggagccgaagtgaagaaacctggggcctctgtgaaggtgtcctgcaaggcttccggctacacctttaccgcctactacctgcactggctgagacaggctccaggccaaggactggaatggatgggctggatgaacaccaacaacggggccaccagatacgcccagaaattccaggacagagtgaccatgaccagagacaccagcatcaacaccgcctacatggaaatgagcggcctgtcctccgacgacaccgccatgtactattgtgccagaggcgacatcagccaggacttcgctgatgtgtggggccagggaacactggtcacagtgtcatct

SEQ ID NO: 542 - protein, 117 aa, synthetic construct
QVQLVQSGAEVKKPGSSVKVSCKASGGTFSSYTINVWRQAPGQGLEWMGGFIPISGTVNYAQKFQGRVTITADESTSTAYMELSSLRSEDTAVYYCARPLDWTEDIWGQGTLVTVSS

SEQ ID NO: 543 - protein, 119 aa, synthetic construct
EVQLVQSGAEVKKPGASVKVSCKASGYTFTAYYLHWLRQAPGQGLEWMGWMNTNNGATRYAQKFQDRVTMTRDTSINTAYMEMSGLSSDDTAMYYCARGDISQDFADVWGQGTLVTVSS

SEQ ID NO: 544 - protein, 8 aa, synthetic construct
GGTFSSYT

SEQ ID NO: 545 - protein, 8 aa, synthetic construct
GYTFTAYY

SEQ ID NO: 546 - protein, 8 aa, synthetic construct
FIPISGTV

SEQ ID NO: 547 - protein, 8 aa, synthetic construct
MNTNNGAT

SEQ ID NO: 548 - protein, 10 aa, synthetic construct
ARPLDWTEDI

SEQ ID NO: 549 - protein, 12 aa, synthetic construct
ARGDISQDFADV

SEQ ID NO: 550 - protein, 11 aa, synthetic construct
RASQDISKYLN

SEQ ID NO: 551 - protein, 7 aa, synthetic construct
SRLHSGV

SEQ ID NO: 552 - protein, 9 aa, synthetic construct
GNTLPYTFG

SEQ ID NO: 553 - protein, 5 aa, synthetic construct
DYGVS

SEQ ID NO: 554 - protein, 16 aa, synthetic construct
VIWGSETTYYNSALKS

SEQ ID NO: 555 - protein, 7 aa, synthetic construct
YAMDYWG

SEQ ID NO: 556 - protein, 119 aa, synthetic construct
EVKLQESGPGLVAPSQSLSVTCTVSGVSLPDYGVSWIQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 557 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 558 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 559 - protein, 11 aa, synthetic construct
KASQNVGTNVA

SEQ ID NO: 560 - protein, 7 aa, synthetic construct
SATYRNS

SEQ ID NO: 561 - protein, 9 aa, synthetic construct
QQYNRYPYT

SEQ ID NO: 562 - protein, 5 aa, synthetic construct
SYWMN

SEQ ID NO: 563 - protein, 17 aa, synthetic construct
QIYPGDGDTNYNGKFKG

SEQ ID NO: 564 - protein, 13 aa, synthetic construct
KTISSVVDFYFDY

SEQ ID NO: 565 - protein, 122 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 566 - protein, 108 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 567 - protein, 245 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 568 - protein, 12 aa, synthetic construct
HYYYGGSYAMDY

SEQ ID NO: 569 - protein, 7 aa, synthetic construct
HTSRLHS

SEQ ID NO: 570 - protein, 9 aa, synthetic construct
QQGNTLPYT

SEQ ID NO: 571 - DNA, 735 bp, synthetic construct
  source 1..735: mol_type = other DNA
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 572 - protein, 6 aa, synthetic construct
DRGSQS

SEQ ID NO: 573 - protein, 6 aa, Synthetic construct
IYSNGD

SEQ ID NO: 574 - protein, 13 aa, synthetic construct
CAGNTGTASKLTF

SEQ ID NO: 575 - protein, 5 aa, synthetic construct
SGDLS

SEQ ID NO: 576 - protein, 6 aa, synthetic construct
YYNGEE

SEQ ID NO: 577 - protein, 19 aa, synthetic construct
CASSGLAGGPVSGANVLTF

SEQ ID NO: 578 - protein, 132 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAGNTGTASKLTFGTGTRLQVTL

SEQ ID NO: 579 - protein, 269 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAGNTGTASKLTFGTGTRLQVTLNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 580 - protein, 137 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGLAGGPVSGANVLTFGAGSRLTVL

SEQ ID NO: 581 - protein, 310 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGLAGGPVSGANVLTFGAGSRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 582 - protein, 7 aa, synthetic construct
NIATNDY

SEQ ID NO: 583 - protein, 5 aa, synthetic construct
GYKTK

SEQ ID NO: 584 - protein, 15 aa, synthetic construct
CLVGGAYTGGFKTIF

SEQ ID NO: 585 - protein, 5 aa, synthetic construct
SGHAT

SEQ ID NO: 586 - protein, 6 aa, synthetic construct
FQNNGV

SEQ ID NO: 587 - protein, 14 aa, synthetic construct
CASSLVAETYEQYF

SEQ ID NO: 588 - protein, 129 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGGAYTGGFKTIFGAGTRLFVKA

SEQ ID NO: 589 - protein, 266 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGGAYTGGFKTIFGAGTRLFVKANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 590 - protein, 133 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLVAETYEQYFGPGTRLTVT

SEQ ID NO: 591 - protein, 306 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLVAETYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 592 - protein, 6 aa, synthetic construct
ATGYPS

SEQ ID NO: 593 - protein, 7 aa, synthetic construct
ATKADDK

SEQ ID NO: 594 - protein, 13 aa, synthetic construct
CALTVGGSYIPTF

SEQ ID NO: 595 - protein, 5 aa, synthetic construct
MGHRA

SEQ ID NO: 596 - protein, 6 aa, synthetic construct
YSYEKL

SEQ ID NO: 597 - protein, 16 aa, synthetic construct
CASSQGGQGWRETQYF

SEQ ID NO: 598 - protein, 131 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALTVGGSYIPTFGRGTSLIVHP

SEQ ID NO: 599 - protein, 268 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALTVGGSYIPTFGRGTSLIVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 600 - protein, 134 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGGQGWRETQYFGPGTRLLVL

SEQ ID NO: 601 - protein, 307 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGGQGWRETQYFGPGTRLLVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 602 - protein, 141 aa, synthetic construct; identical to SEQ ID NO: 18

SEQ ID NO: 603 - protein, 137 aa, synthetic construct
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 604 - protein, 177 aa, synthetic construct; identical to SEQ ID NO: 70

SEQ ID NO: 605 - protein, 173 aa, synthetic construct
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 606 - protein, 179 aa, synthetic construct; identical to SEQ ID NO: 28

SEQ ID NO: 607 - protein, 15 aa, synthetic construct
CAVSVGPGNTGKLIF

SEQ ID NO: 608 - protein, 13 aa, synthetic construct
CASSLGVLGLRYF

SEQ ID NO: 609 - protein, 14 aa, synthetic construct
CAENSGGSNYKLTF

SEQ ID NO: 610 - protein, 13 aa, synthetic construct
CASSWERAGKAFF

SEQ ID NO: 611 - protein, 13 aa, synthetic construct
CAMSVFIYSTFIF

SEQ ID NO: 612 - protein, 12 aa, synthetic construct
CASSGRQETQYF

SEQ ID NO: 613 - protein, 12 aa, synthetic construct
CATDAYTRQLTF

SEQ ID NO: 614 - protein, 14 aa, synthetic construct
CASGGPGANRPQHF

SEQ ID NO: 615 - protein, 15 aa, synthetic construct
CAYRSLWGSGYALNF

SEQ ID NO: 616 - protein, 13 aa, synthetic construct
CSVEKGAQETQYF

SEQ ID NO: 617 - protein, 12 aa, synthetic construct
CAALFGNEKLTF

SEQ ID NO: 618 - protein, 14 aa, synthetic construct
CATLQGWSYNEQFF

SEQ ID NO: 619 - protein, 12 aa, synthetic construct
CALSTGGFKTIF

SEQ ID NO: 620 - protein, 19 aa, synthetic construct
CSVLGPGTGGRGANYGYTF

SEQ ID NO: 621 - protein, 12 aa, synthetic construct
CATTIDGQKLLF

SEQ ID NO: 622 - protein, 13 aa, synthetic construct
CSASDRGSGELFF

SEQ ID NO: 623 - protein, 12 aa, synthetic construct
CATDAYNARLMF

SEQ ID NO: 624 - protein, 14 aa, synthetic construct
CSVVASGSVDTQYF

SEQ ID NO: 625 - protein, 12 aa, synthetic construct
CAGPAGAQKLVF

SEQ ID NO: 626 - protein, 15 aa, synthetic construct
CASSPVPYSGNTIYF

SEQ ID NO: 627 - protein, 11 aa, synthetic construct
CAVSKSDKIIF

SEQ ID NO: 628 - protein, 15 aa, synthetic construct
CATSEGGSTGTEAFF

SEQ ID NO: 629 - protein, 13 aa, synthetic construct
CALNRNTGNQFYF

SEQ ID NO: 630 - protein, 13 aa, synthetic construct
CASSIPQGNGYTF

SEQ ID NO: 631 - protein, 275 aa, synthetic construct
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSVGPGNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 632 - protein, 308 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLGVLGLRYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 633 - protein, 274 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENSGGSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 634 - protein, 308 aa, synthetic construct
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSWERAGKAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 635 - protein, 274 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSVFIYSTFIFGSGTRLSVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 636 - protein, 307 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSGRQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 637 - protein, 271 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYTRQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 638 - protein, 309 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASGGPGANRPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 639 - protein, 277 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSLWGSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 640 - protein, 306 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVEKGAQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 641 - protein, 271 aa, synthetic construct
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAALFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 642 - protein, 310 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCATLQGWSYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 643 - protein, 268 aa, synthetic construct
MKPTLISVLVIIFILRGTRAQRVTQPEKLLSVFKGAPVELKCNYSYSGSPELFWYVQYSRQRLQLLLRHISRESIKGFTADLNKGETSFHLKKPFAQEEDSAMYYCALSTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 644 - protein, 312 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVLGPGTGGRGANYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 645 - protein, 271 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATTIDGQKLLFARGTMLKVDLNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 646 - protein, 307 aa, synthetic construct
MLLLLLLLGPAGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSASDRGSGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 647 - protein, 271 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 648 - protein, 307 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVVASGSVDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 649 - protein, 271 aa, synthetic construct
MVLKFSVSILWIQLAWVSTQLLEQSPQFLSIQEGENLTVYCNSSSVFSSLQWYRQEPGALEGPVLLVTVVTGGEVKKLKRLTFQFGDARKDSSLHITAAQPGDTGLYLCAGPAGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 650 - protein, 309 aa, synthetic construct
MSISLLCCAAFPLLWAGPVNAGVTQTPKFRLKIGQSMTLQCTQDMNHNYMYWYRQDPGMGLKLIYYSVGAGITDKGEVPNGYNVSRSTTEDFPLRLELAAPSQTSVYFCASSPVPYSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 651 - protein, 267 aa, synthetic construct
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSKSDKIIFGKHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 652 - protein, 310 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSEGGSTGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 653 - protein, 272 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALNRNTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 654 - protein, 308 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIPQGNGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 655 - DNA, 829 bp, synthetic construct
  source 1..829: mol_type = other DNA
atgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgtaggtcctggcaacacaggcaaactaatctttgggcaagggacaactttacaagtaaaaccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaaggtggccgggtttaatctgctcatgacgctgcggctgtggtccagctaa

SEQ ID NO: 656 - DNA, 927 bp, synthetic construct
  source 1..927: mol_type = other DNA
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttgggggttctaggcctgcggtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 657 - DNA, 825 bp, synthetic construct
  source 1..825: mol_type = other DNA; note = N is g nucleotide
atggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctnnaaaanntcctcaattcattatanacattcnttcaaatatnnacaaaanncaannccaaagagtcaccgttttattgaataagacagtgaaacatctctctctgcaaattgcagctactcaacctggagactcagctgtctacttttgtgcagagaatagtggaggtagcaactataaactgacatttggaaaaggaactctcttaaccgtgaatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 658 - DNA, 927 bp, synthetic construct
  source 1..927: mol_type = other DNA
atgggccctgggctcctctgctgggtgctgctttgtctcctgggagcaggctcagtggagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagctgggaaagggcggggaaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 659 - DNA, 825 bp, synthetic construct
  source 1..825: mol_type = other DNA; note = N is g nucleotide
atgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgagcgtctttatttatagcacattcatctttgggagtgggacaagattatcagtaaaacctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtgnccgggtttaatctgctcatgacgctgcggctgtggtccagctaa

SEQ ID NO: 660 - DNA, 924 bp, synthetic construct
  source 1..924: mol_type = other DNA
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagctctgggaggcaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 661 - DNA, 696 bp, synthetic construct
  source 1..696: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgcgtacacaaggcaactgacctttggatctgggacacaattgactgttttacctgatatccagaaccctgaccctgccgtgtacgacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 662 - DNA, 930 bp, synthetic construct
  source 1..930: mol_type = other DNA; note = N is g nucleotide
atgagcaaccaggtgctctgctgtgtggtcctttgtctcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtgggggaccgggggctaacaggccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatnnccatnntcaananaaannatttctaa

SEQ ID NO: 663 - DNA, 834 bp, synthetic construct
  source 1..834: mol_type = other DNA; note = N is g nucleotide
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcctttgggggtccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctnaaantggccgggtttaatctgctcatgacgctgcggctgtggtccanctaa

SEQ ID NO: 664 - DNA, 921 bp, synthetic construct
  source 1..921: mol_type = other DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgaaaagggcgcgcaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 665 - DNA, 816 bp, synthetic construct
  source 1..816: mol_type = other DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcactctttggaaatgagaaattaacctttgggactggaacaagactcaccatcatacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 666 - DNA, 933 bp, synthetic construct
  source 1..933: mol_type = other DNA
atgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccaccctacagggttggtcttataatgagcagttcttcgggccagggacacggctcaccgtgctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 667 - DNA, 807 bp, synthetic construct
  source 1..807: mol_type = other DNA
atgaagcccaccctcatctcagtgcttgtgataatatttatactcagaggaacaagagcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgctctaagtactggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 668 - DNA, 939 bp, synthetic construct
  source 1..939: mol_type = other DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttttgggtcccgggacagggggacgaggagctaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 669 - DNA, 816 bp, synthetic construct
  source 1..816: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctaccactatagatggccagaagctgctctttgcaaggggaaccatgttaaaggtggatcttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 670 - DNA, 924 bp, synthetic construct
  source 1..924: mol_type = other DNA; note = N is g nucleotide
atgctgctgcttctgctgcttctggggccagcaggctccgggcttggtgctgtcgtctctcaacatccgagcagggttatctgtaagagtggaacctctgtgaagatcgagtgccgttccctggactttcaggccacaactatgttttggtatcgtcagttcccgaaaaagagtctcatgctgatggcaacttccaatgagggctccaaggccacatacgagcaaggcgtcgagaaggacaantttctcatcaaccatncaancctnaccttntccactctnacantnaccantncccatcctgaagacagcagcttctacatctgcagtgcttctgacagggggtccggggagctgttttttggagaaggctctaggctgaccgtactggaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 671 - DNA, 816 bp, synthetic construct
  source 1..816: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgcatacaatgccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 672 - DNA, 924 bp, synthetic construct
  source 1..924: mol_type = other DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgtggcaagcgggagtgtagatacgcagtattttggcccaggcacccggctgacagtgctcgaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 673 - DNA, 810 bp, synthetic construct
  source 1..810: mol_type = other DNA
atggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggtcctgcgggagcccagaagctggtatttggccaaggaaccaggctgactatcaacccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 674 - DNA, 933 bp, synthetic construct
  source 1..933: mol_type = other DNA
atgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagccccgtaccctactctggaaacaccatatattttggagagggaagttggctcactgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 675 - DNA, 816 bp, synthetic construct
  source 1..816: mol_type = other DNA
atgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtaagagtgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 676 - DNA, 933 bp, synthetic construct
  source 1..933: mol_type = other DNA
atggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgagggggggagtacgggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 677 - DNA, 819 bp, synthetic construct
  source 1..819: mol_type = other DNA
atgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgaataggaacaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 678 - DNA, 926 bp, synthetic construct
  source 1..926: mol_type = other DNA; note = N is g nucleotide
atgagcaaccaggtgctctgctgtgtggtcctttgtctcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtatcccacagggcaatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatnanatcctnctaggaaggccaccctgtatgctgtgctggtcagcgcccttgtgttgatggccatggtcaagagaaaggatttctaa

SEQ ID NO: 679 - DNA, 45 bp, synthetic construct
  source 1..45: mol_type = other DNA
tgtgctgtgagtgtaggtcctggcaacacaggcaaactaatcttt

SEQ ID NO: 680 - DNA, 39 bp, synthetic construct
  source 1..39: mol_type = other DNA
tgtgccagcagtttgggggttctaggcctgcggtacttc

SEQ ID NO: 681 - DNA, 42 bp, synthetic construct
  source 1..42: mol_type = other DNA
tgtgcagagaatagtggaggtagcaactataaactgacattt

SEQ ID NO: 682 - DNA, 39 bp, synthetic construct
  source 1..39: mol_type = other DNA
tgtgccagcagctgggaaagggcggggaaagctttcttt

SEQ ID NO: 683 - DNA, 39 bp, synthetic construct
  source 1..39: mol_type = other DNA
tgtgcaatgagcgtctttatttatagcacattcatcttt

SEQ ID NO: 684 - DNA, 36 bp, synthetic construct
  source 1..36: mol_type = other DNA
tgtgccagctctgggaggcaagagacccagtacttc

SEQ ID NO: 685 - DNA, 36 bp, synthetic construct
  source 1..36: mol_type = other DNA
tgtgctacggacgcgtacacaaggcaactgaccttt

SEQ ID NO: 686 - DNA, 42 bp, synthetic construct
  source 1..42: mol_type = other DNA
tgtgccagtgggggaccgggggctaacaggccccagcatttt

SEQ ID NO: 687 - DNA, 45 bp, synthetic construct
  source 1..45: mol_type = other DNA
tgtgcttataggagcctttgggggtccgggtatgcactcaacttc

SEQ ID NO: 688 - DNA, 39 bp, synthetic construct
  source 1..39: mol_type = other DNA
tgcagcgttgaaaagggcgcgcaagagacccagtacttc

SEQ ID NO: 689 - DNA, 36 bp, synthetic construct
  source 1..36: mol_type = other DNA
tgtgcagcactctttggaaatgagaaattaaccttt

SEQ ID NO: 690 - DNA, 42 bp, synthetic construct
  source 1..42: mol_type = other DNA
tgtgccaccctacagggttggtcttataatgagcagttcttc

SEQ ID NO: 691 - DNA, 36 bp, synthetic construct
  source 1..36: mol_type = other DNA
tgtgctctaagtactggaggcttcaaaactatcttt

SEQ ID NO: 692 - DNA, 57 bp, synthetic construct
  source 1..57: mol_type = other DNA
tgcagcgttttgggtcccgggacagggggacgaggagctaactatggctacaccttc

SEQ ID NO: 693 - DNA, 36 bp, synthetic construct
  source 1..36: mol_type = other DNA
tgtgctaccactatagatggccagaagctgctcttt

SEQ ID NO: 694 - DNA, 39 bp, synthetic construct
  source 1..39: mol_type = other DNA
tgcagtgcttctgacagggggtccggggagctgtttttt

SEQ ID NO: 695 - DNA, 36 bp, synthetic construct
  source 1..36: mol_type = other DNA
tgtgctacggacgcatacaatgccagactcatgttt

SEQ ID NO: 696 - DNA, 42 bp, synthetic construct
  source 1..42: mol_type = other DNA
tgcagcgttgtggcaagcgggagtgtagatacgcagtatttt

SEQ ID NO: 697 - DNA, 36 bp, synthetic construct
  source 1..36: mol_type = other DNA
tgtgcaggtcctgcgggagcccagaagctggtattt

SEQ ID NO: 698 - DNA, 45 bp, synthetic construct
  source 1..45: mol_type = other DNA
tgtgccagcagccccgtaccctactctggaaacaccatatatttt

SEQ ID NO: 699 - DNA, 33 bp, synthetic construct
  source 1..33: mol_type = other DNA
tgtgctgtgagtaagagtgacaagatcatcttt

SEQ ID NO: 700 - DNA, 45 bp, synthetic construct
  source 1..45: mol_type = other DNA
tgtgccaccagtgagggggggagtacgggcactgaagctttcttt

SEQ ID NO: 701 - DNA, 39 bp, synthetic construct
  source 1..39: mol_type = other DNA
tgtgctctgaataggaacaccggtaaccagttctatttt

SEQ ID NO: 702 - DNA, 39 bp, synthetic construct
  source 1..39: mol_type = other DNA
tgtgccagtagtatcccacagggcaatggctacaccttc

SEQ ID NO: 703 - protein, 141 aa, synthetic construct; identical to SEQ ID NO: 317

SEQ ID NO: 704 - protein, 140 aa, synthetic construct; identical to SEQ ID NO: 405

SEQ ID NO: 705 - protein, 140 aa, synthetic construct; identical to SEQ ID NO: 404

SEQ ID NO: 706 - protein, 140 aa, synthetic construct; identical to SEQ ID NO: 405

SEQ ID NO: 707 - protein, 140 aa, synthetic construct; identical to SEQ ID NO: 407

SEQ ID NO: 708 - protein, 137 aa, synthetic construct; identical to SEQ ID NO: 408

SEQ ID NO: 709 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 710 - protein, 140 aa, synthetic construct; identical to SEQ ID NO: 409

SEQ ID NO: 711 - protein, 140 aa, synthetic construct; identical to SEQ ID NO: 410

SEQ ID NO: 712 - protein, 239 aa, synthetic construct; identical to SEQ ID NO: 412

SEQ ID NO: 713 - protein, 177 aa, synthetic construct; identical to SEQ ID NO: 70

SEQ ID NO: 714 - protein, 179 aa, synthetic construct; identical to SEQ ID NO: 28

SEQ ID NO: 715 - protein, 179 aa, synthetic construct; identical to SEQ ID NO: 28

SEQ ID NO: 716 - protein, 179 aa, synthetic construct; identical to SEQ ID NO: 29

SEQ ID NO: 717 - protein, 179 aa, synthetic construct; identical to SEQ ID NO: 419

SEQ ID NO: 718 - protein, 179 aa, synthetic construct; identical to SEQ ID NO: 28

SEQ ID NO: 719 - protein, 179 aa, synthetic construct; identical to SEQ ID NO: 423

SEQ ID NO: 720 - protein, 179 aa, synthetic construct; identical to SEQ ID NO: 432

SEQ ID NO: 721 - protein, 276 aa, synthetic construct; identical to SEQ ID NO: 435

SEQ ID NO: 722 - protein, 269 aa, synthetic construct; identical to SEQ ID NO: 436

SEQ ID NO: 723 - protein, 258 aa, synthetic construct; identical to SEQ ID NO: 437

SEQ ID NO: 724 - protein, 210 aa, synthetic construct; identical to SEQ ID NO: 438

SEQ ID NO: 725 - protein, 189 aa, synthetic construct; identical to SEQ ID NO: 439

SEQ ID NO: 726 - protein, 173 aa, synthetic construct; identical to SEQ ID NO: 440

SEQ ID NO: 727 - protein, 153 aa, synthetic construct; identical to SEQ ID NO: 441

SEQ ID NO: 728 - protein, 153 aa, synthetic construct; identical to SEQ ID NO: 441

SEQ ID NO: 729 - protein, 115 aa, synthetic construct
KNQVEQSPQSLIILEGKNCTLQCNYTVSPFSNLRWYKQDTGRGPVSLTIMTFSENTKSNGRYTATLDADTKQSSLHITASQLSDSASYICVVSDRGSTLGRLYFGRGTQLTVWPD

SEQ ID NO: 730 - protein, 111 aa, synthetic construct
DIYQTPRYLVIGTGKKITLECSQTMGHDKMYWYQQDPGMELHLIHYSYGVNSTEKGDLSSESTVSRIRTEHFPLTLESARPSHTSQYLCASSENIGTAYEQYFGPGTRLTV

SEQ ID NO: 731 - protein, 119 aa, synthetic construct
GVTQSPTHLIKTRGQQATLRCSPISGHTSVYWYQQALGLGLQFLLWYDEGEERNRGNFPPRFSGRQFPNYSSELNVNALELEDSALYLCASSLGGPRGLAGLRGDEQFFGPGTRLTVLE

SEQ ID NO: 732 - protein, 117 aa, synthetic construct
GVTQSPTHLIKTRGQQATLRCSPISGHTSVYWYQQALGLGLQFLLWYDEGEERNRGNFPPRFSGRQFPNYSSELNVNALELEDSALYLCASSLGGDELGADGNEQFFGPGTRLTVLE

SEQ ID NO: 733 - protein, 115 aa, synthetic construct
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVNARLMFGDGTQLVVKPN

SEQ ID NO: 734 - protein, 118 aa, synthetic construct
EADIYQTPRYLVIGTGKKITLECSQTMGHDKMYWYQQDPGMELHLIHYSYGVNSTEKGDLSSESTVSRIRTEHFPLTLESARPSHTSQYLCASSEARGLAEFTDTQYFGPGTRLTVLE

SEQ ID NO: 735 - protein, 114 aa, synthetic construct
QEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPY

SEQ ID NO: 736 - protein, 114 aa, synthetic construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLE

SEQ ID NO: 737 - protein, 110 aa, synthetic construct
ILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIP

SEQ ID NO: 738 - protein, 113 aa, synthetic construct
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSFQTGASYGYTFGSGTRLTVLE

SEQ ID NO: 739 - protein, 120 aa, synthetic construct
AIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPR

SEQ ID NO: 740 - protein, 126 aa, synthetic construct
AGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQL

SEQ ID NO: 741 - protein, 6 aa, synthetic construct
QDVNTA

SEQ ID NO: 742-743 - skipped (2 entries)

SEQ ID NO: 744 - protein, 11 aa, synthetic construct
QQYSRYSPVTF

SEQ ID NO: 745 - protein, 10 aa, synthetic construct
QQQSSTPVTF

SEQ ID NO: 746 - protein, 10 aa, synthetic construct
QQSSYYPNTF

SEQ ID NO: 747 - protein, 10 aa, synthetic construct
QQQWSSPDTF

SEQ ID NO: 748 - protein, 10 aa, synthetic construct
QQSNAYPITF

SEQ ID NO: 749 - protein, 9 aa, synthetic construct
GFNVYASGM

SEQ ID NO: 750 - protein, 9 aa, synthetic construct
GFNVYQSDM

SEQ ID NO: 751 - protein, 9 aa, synthetic construct
GFNLYQRDM

SEQ ID NO: 752 - protein, 9 aa, synthetic construct
GFNLSYYDM

SEQ ID NO: 753 - protein, 9 aa, synthetic construct
GFNLNSYYM

SEQ ID NO: 754 - protein, 10 aa, synthetic construct
KIYPDSDYTY

SEQ ID NO: 755 - protein, 10 aa, synthetic construct
TIWPYSGYTY

SEQ ID NO: 756 - protein, 10 aa, synthetic construct
GLLYGSDHTE

SEQ ID NO: 757 - protein, 10 aa, synthetic construct
LIYYGSGYTY

SEQ ID NO: 758 - protein, 10 aa, synthetic construct
MIIPGYGYTN

SEQ ID NO: 759 - protein, 14 aa, synthetic construct
SRDSSFYYVYAMDY

SEQ ID NO: 760 - protein, 10 aa, synthetic construct
SRDGMYAFDY

SEQ ID NO: 761 - protein, 11 aa, synthetic construct
SRATYEEAFDY

SEQ ID NO: 762 - protein, 10 aa, synthetic construct
SRGYVSGMDY

SEQ ID NO: 763 - protein, 10 aa, synthetic construct
SRSYYMYMDY

SEQ ID NO: 764 - protein, 6 aa, synthetic construct
QDVNTA

SEQ ID NO: 765 - skipped

SEQ ID NO: 766 - protein, 10 aa, synthetic construct
QQVIYYPFTF

SEQ ID NO: 767 - protein, 10 aa, synthetic construct
QQYDYYPFTF

SEQ ID NO: 768 - protein, 10 aa, synthetic construct
QQSIYYPFTF

SEQ ID NO: 769 - protein, 10 aa, synthetic construct
QQSSYSPWTF

SEQ ID NO: 770 - protein, 10 aa, synthetic construct
QQSFSTPITF

SEQ ID NO: 771 - protein, 10 aa, synthetic construct
QQGEYSPLTF

SEQ ID NO: 772 - protein, 10 aa, synthetic construct
QQTYYTPVTF

SEQ ID NO: 773 - protein, 9 aa, synthetic construct
GFNLYSYAI

SEQ ID NO: 774 - protein, 9 aa, synthetic construct
GFNISYEAM

SEQ ID NO: 775 - protein, 9 aa, synthetic construct
GFNLYTSQM

SEQ ID NO: 776 - protein, 9 aa, synthetic construct
GFNVFGYAI

SEQ ID NO: 777 - protein, 9 aa, synthetic construct
GFNISPWDM

SEQ ID NO: 778 - protein, 9 aa, synthetic construct
GFNISEYLM

SEQ ID NO: 779 - protein, 9 aa, synthetic construct
GFNVFESAM

SEQ ID NO: 780 - protein, 9 aa, synthetic construct
GFNISHYVM

SEQ ID NO: 781 - protein, 10 aa, synthetic construct
LLYPDYGVTS

SEQ ID NO: 782 - protein, 10 aa, synthetic construct
LIYPNHGITS

SEQ ID NO: 783 - protein, 10 aa, synthetic construct
LVYPGYYVTS

SEQ ID NO: 784 - protein, 10 aa, synthetic construct
EVYPGYDVTS

SEQ ID NO: 785 - protein, 10 aa, synthetic construct
QLYPSSGYTN

SEQ ID NO: 786 - protein, 10 aa, synthetic construct
LLPPGLSYTN

SEQ ID NO: 787 - protein, 10 aa, synthetic construct
WVYGSYDYTY

SEQ ID NO: 788 - protein, 10 aa, synthetic construct
DFYPHSDSTY

SEQ ID NO: 789 - protein, 20 aa, synthetic construct
SRYRSYEYSVSSYSYSAMDY

SEQ ID NO: 790 - protein, 10 aa, synthetic construct
SRYSSSAMDY

SEQ ID NO: 791 - protein, 13 aa, synthetic construct
SRGAYYYSSAMDY

SEQ ID NO: 792 - protein, 11 aa, synthetic construct
SRYSWAGAFDY

SEQ ID NO: 793 - protein, 10 aa, synthetic construct
SRSVYWSLDY

SEQ ID NO: 794 - protein, 10 aa, synthetic construct
SRYGYYAFDY

SEQ ID NO: 795 - protein, 12 aa, synthetic construct
SRSFAYFQAMDY

SEQ ID NO: 796 - protein, 10 aa, synthetic construct
SRYQSYSFDY

SEQ ID NO: 797 - protein, 10 aa, synthetic construct
QQASRQPYTF

SEQ ID NO: 798 - protein, 10 aa, synthetic construct
QQAVSYPWTF

SEQ ID NO: 799 - protein, 10 aa, synthetic construct
QQTSSYPITF

SEQ ID NO: 800 - protein, 10 aa, synthetic construct
QQSWYSPSTF

SEQ ID NO: 801 - protein, 10 aa, synthetic construct
QQSYYAPITF

SEQ ID NO: 802 - protein, 10 aa, synthetic construct
QQSYYSPWTF

SEQ ID NO: 803 - protein, 10 aa, synthetic construct
QQAYYPPWTF

SEQ ID NO: 804 - protein, 11 aa, synthetic construct
QQSYSSGPVTF

SEQ ID NO: 805 - protein, 10 aa, synthetic construct
QQTYYYPFTF

SEQ ID NO: 806 - protein, 12 aa, synthetic construct
QQSYYPYYPWTF

SEQ ID NO: 807 - protein, 9 aa, synthetic construct
QQYDRPITF

SEQ ID NO: 808 - protein, 9 aa, synthetic construct
GFNFSESGM

SEQ ID NO: 809 - protein, 9 aa, synthetic construct
GFNISSSGI

SEQ ID NO: 810 - protein, 9 aa, synthetic construct
GFNIYWYGM

SEQ ID NO: 811 - protein, 9 aa, synthetic construct
GFNISASGM

SEQ ID NO: 812 - protein, 9 aa, synthetic construct
GFNFSYYGM

SEQ ID NO: 813 - protein, 9 aa, synthetic construct
GFNISYSNI

SEQ ID NO: 814 - protein, 9 aa, synthetic construct
GFNVSRWAM

SEQ ID NO: 815 - protein, 9 aa, synthetic construct
GFNFSYGGI

SEQ ID NO: 816 - protein, 9 aa, synthetic construct
GFNLYAWGM

SEQ ID NO: 817 - protein, 9 aa, synthetic construct
GFNVSHSAM

SEQ ID NO: 818 - protein, 9 aa, synthetic construct
GFNIYYEAM

SEQ ID NO: 819 - protein, 10 aa, synthetic construct
HFSGDSGYTY

SEQ ID NO: 820 - protein, 10 aa, synthetic construct
MVYGGSGYTN

SEQ ID NO: 821 - protein, 10 aa, synthetic construct
QVYPWSGFTY

SEQ ID NO: 822 - protein, 10 aa, synthetic construct
WIWGGSSYTY

SEQ ID NO: 823 - protein, 10 aa, synthetic construct
WIYPFSGYTN

SEQ ID NO: 824 - protein, 10 aa, synthetic construct
MIYGTRGGTY

SEQ ID NO: 825 - protein, 10 aa, synthetic construct
RVYPSGYLTY

SEQ ID NO: 826 - protein, 10 aa, synthetic construct
MIYPLTGYTN

SEQ ID NO: 827 - protein, 10 aa, synthetic construct
LVYGGWGSTS

SEQ ID NO: 828 - protein, 10 aa, synthetic construct
TVHPDWGNTY

SEQ ID NO: 829 - protein, 10 aa, synthetic construct
QIYPWNDYTY

SEQ ID NO: 830 - protein, 12 aa, synthetic construct
SRYMYYSGYFDY

SEQ ID NO: 831 - protein, 12 aa, synthetic construct
SRWAHYSAYMDY

SEQ ID NO: 832 - protein, 11 aa, synthetic construct
SRDYYSYSLDY

SEQ ID NO: 833 - protein, 11 aa, synthetic construct
SRGQYLSYMDY

SEQ ID NO: 834 - protein, 11 aa, synthetic construct
SREYYSRAFDY

SEQ ID NO: 835 - protein, 10 aa, synthetic construct
SRYYSYAMDY

SEQ ID NO: 836 - protein, 10 aa, synthetic construct
SRNMQSYMDY

SEQ ID NO: 837 - protein, 10 aa, synthetic construct
SRDYYYSVDV

SEQ ID NO: 838 - protein, 15 aa, synthetic construct
SRAGSSKMSAGAFDY

SEQ ID NO: 839 - protein, 12 aa, synthetic construct
SRWQQYYYSFDY

SEQ ID NO: 840 - protein, 11 aa, synthetic construct
SRNYYAATMDY

SEQ ID NO: 841 - protein, 10 aa, synthetic construct
QQSYTSPLTF

SEQ ID NO: 842 - protein, 11 aa, synthetic construct
QQYWYYYPITF

SEQ ID NO: 843 - protein, 10 aa, synthetic construct
QQSYYAPITF

SEQ ID NO: 844 - protein, 11 aa, synthetic construct
QQYYLYQPITF

SEQ ID NO: 845 - protein, 10 aa, synthetic construct
QQYSNYPLTF

SEQ ID NO: 846 - protein, 10 aa, synthetic construct
QQYASDPITF

SEQ ID NO: 847 - protein, 10 aa, synthetic construct
QQYSYDPITF

SEQ ID NO: 848 - protein, 10 aa, synthetic construct
QQYIYDPVTF

SEQ ID NO: 849 - protein, 10 aa, synthetic construct
QQLMYDPITF

SEQ ID NO: 850 - protein, 9 aa, synthetic construct
GFNIYYGVM

SEQ ID NO: 851 - protein, 9 aa, synthetic construct
GFNIYSYDM

SEQ ID NO: 852 - protein, 9 aa, synthetic construct
GFNVQWSHM

SEQ ID NO: 853 - protein, 9 aa, synthetic construct
GFNIGMYTM

SEQ ID NO: 854 - protein, 9 aa, synthetic construct
GFNVFYGSM

SEQ ID NO: 855 - protein, 9 aa, synthetic construct
GFNLDYGWM

SEQ ID NO: 856 - protein, 9 aa, synthetic construct
GFNFSYSAM

SEQ ID NO: 857 - protein, 9 aa, synthetic construct
GFNVDWAWM

SEQ ID NO: 858 - protein, 9 aa, synthetic construct
GFNFGTYWM

SEQ ID NO: 859 - protein, 10 aa, synthetic construct
MIYPDSSWTY

SEQ ID NO: 860 - protein, 9 aa, synthetic construct
ISPGGSYTY

SEQ ID NO: 861 - protein, 10 aa, synthetic construct
RLSPPSGYTN

SEQ ID NO: 862 - protein, 10 aa, synthetic construct
LVYPDSGYTN

SEQ ID NO: 863 - protein, 10 aa, synthetic construct
FIGPDSTYTY

SEQ ID NO: 864 - protein, 10 aa, synthetic construct
WVVPGSDYTD

SEQ ID NO: 865 - protein, 10 aa, synthetic construct
DVVPDGDWTY

SEQ ID NO: 866 - protein, 10 aa, synthetic construct
WVVGGSDYTY

SEQ ID NO: 867 - protein, 10 aa, synthetic construct
WFLPDYDYTL

SEQ ID NO: 868 - protein, 19 aa, synthetic construct
SRDQDFHYMNYYLSYALDY

SEQ ID NO: 869 - protein, 11 aa, synthetic construct
SRSAFTGYFDV

SEQ ID NO: 870 - protein, 16 aa, synthetic construct
SRLILSKGGYGWAMDY

SEQ ID NO: 871 - protein, 10 aa, synthetic construct
SRYTWQSMDY

SEQ ID NO: 872 - protein, 12 aa, synthetic construct
SRDLGSAYAMDY

SEQ ID NO: 873 - protein, 10 aa, synthetic construct
SRFHYTAFDV

SEQ ID NO: 874 - protein, 9 aa, synthetic construct
SRGWYALDY

SEQ ID NO: 875 - protein, 10 aa, synthetic construct
SRSYYYAFDY

SEQ ID NO: 876 - protein, 10 aa, synthetic construct
SRHGEYAFDY

SEQ ID NO: 877 - protein, 245 aa, synthetic construct
DIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSS

SEQ ID NO: 878 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQQSSTPVTFGQGTKVEIKRTGGGSGGGGSGGGASEVQLVESGGGLVQPGGSLRLSCAASGFNVYQSDMHWVRQAPGKGLEWVATIWPYSGYTYYADSVKGRFTISADTSKNTAYLQMNSLRAEDTAVYYCSRDGMYAFDYWGQGTLVTVSS

SEQ ID NO: 879 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSSYYPNTFGQGTKVEIKRTGGGSGGGGSGGGASEVQLVESGGGLVQPGGSLRLSCAASGFNLYQRDMHWVRQAPGKGLEWVAGLLYGSDHTEYADSVKGRFTISADTSKNTAYLQMNSLRAEDTAVYYCSRATYEEAFDYWGQGTLVTVSS

SEQ ID NO: 880 - protein, 241 aa, synthetic construct
DIQMTQSPSSLSASVGDRVTITCRASQDVNTAVAWYQQKPGKAPKLLIYSASFLYSGVPSRFSGSRSGTDFTLTITSLQPEDFATYYCQQQWSSPDTFGQGTKVEIKRTGGGSGGGGSGGGASEVQLVESGGGLVQPGGSLRLSCAASGFNLSYYDMHWVRQAPGKGLEWVALIYYGSGYTYYADSVKGRFTISADTSKNTAYLQMNSLRAEDTAVYYCSRGSYVSGMDYWGQGTLVTVSS

SEQ ID NO: 881 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSNAYPITFGQGTKVEIKRTGGGSGGGGSGGGASEVQLVESGGGLVQPGGSLRLSCAASGFNLNSYYMHWVRQAPGKGLEWVAMIIPGYGYTNYADSVKGRFTISADTSKNTAYLQMNSLRAEDTAVYYCSRSYYMYMDYWGQGTLVTVSS

SEQ ID NO: 882 - protein, 250 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQVIYYPFTFGQGTKVEIKRTGGGSGGGGSGGGASEVQLVESGGGLVQPGGSLRLSCAASGFNLYSYAIHWVRQAPGKGLEWVALLYPDYGVTSYADSVKGRFTISADTSKNTAYLQMNSLRAEDTAVYYCSRYRSYEYSVSSYSYSAMDYWGQGTLVTVSS

SEQ ID NO: 883 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYDYYPFTFGQGTKVEIKRTGGGSGGGGSGGGASEVQLVESGGGLVQPGGSLRLSCAASGFNISYEAMHWVRQAPGKGLEWVALIYPNHGITSYADSVKGRFTISADTSKNTAYLQMNSLRAEDTAVYYCSRYSSSAMDYWGQGTLVTVSS

SEQ ID NO: 884 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSIYYPFTFGQGTKVEIKRTGGGSGGGGSGGGASEVQLVESGGGLVQPGGSLRLSCAASGFNLYTSQMHWVRQAPGKGLEWVALVYPGYYVTSYADSVKGRFTISADTSKNTAYLQMNSLRAEDTAVYYCSRGAYYYSSAMDYWGQGTLVTVSS

SEQ ID NO: 885 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYDYYPFTFGQGTKVEIKRTGGGSGGGGSGGGASEVQLVESGGGLVQPGGSLRLSCAASGFNVFGYAIHWVRQAPGKGLEWVAEVYPGYDVTSYADSVKGRFTISADTSKNTAYLQMNSLRAEDTAVYYCSRYSWAGAFDYWGQGTLVTVSS

SEQ ID NO: 886 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSSYSPWTFGQGTKVEIKRTGGGSGGGGSGGGASEVQLVESGGGLVQPGGSLRLSCAASGFNISPWDMHWVRQAPGKGLEWVAQLYPSSGYTNYADSVKGRFTISADTSKNTAYLQMNSLRAEDTAVYYCSRSVYWSLDYWGQGTLVTVSS

SEQ ID NO: 887 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSFSTPITFGQGTKVEIKRTGGGSGGGGSGGGASEVQLVESGGGLVQPGGSLRLSCAASGFNISEYLMHWVRQAPGKGLEWVALLPPGLSYTNYADSVKGRFTISADTSKNTAYLQMNSLRAEDTAVYYCSRYGYYAFDYWGQGTLVTVSS

SEQ ID NO: 888 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQGEYSPLTFGQGTKVEIKRTGGGSGGGGSGGGASEVQLVESGGGLVQPGGSLRLSCAASGFNVFESAMHWVRQAPGKGLEWVAWVYGSYDYTYYADSVKGRFTISADTSKNTAYLQMNSLRAEDTAVYYCSRSFAYFQAMDYWGQGTLVTVSS

SEQ ID NO: 889 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQTYYTPVTFGQGTKVEIKRTGGGSGGGGSGGGASEVQLVESGGGLVQPGGSLRLSCAASGFNISHYVMHWVRQAPGKGLEWVADFYPHSDSTYYADSVKGRFTISADTSKNTAYLQMNSLRAEDTAVYYCSRYQSYSFDYWGQGTLVTVSS

SEQ ID NO: 890 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQASRQPYTFGQGTKVEIKRTGGGSGGGGSGGGASEVQLVESGGGLVQPGGSLRLSCAASGFNFSESGMHWVRQAPGKGLEWVAHFSGDSGYTYYADSVKGRFTISADTSKNTAYLQMNSLRAEDTAVYYCSRYMYYSGYFDYWGQGTLVTVSS

SEQ ID NO: 891 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQAVSYPWTFGQGTKVEIKRTGGGSGGGGSGGGASEVQLVESGGGLVQPGGSLRLSCAASGFNISSSGIHWVRQAPGKGLEWVAMVYGGSGYTNYADSVKGRFTISADTSKNTAYLQMNSLRAEDTAVYYCSRWAHYSAYMDYWGQGTLVTVSS

SEQ ID NO: 892 - protein, 241 aa, synthetic construct
DIQMTQSPSSLSASVGDRVTITCRASQDVNTANAWYQQKPGKAPKLLIYSASFLYSGVPSRFSGSRSGTDFTLTISSLQPEDFATYYCQQTSSYPITFGQGTKVEIKRTGGGSGGGGSGGGASEVQLVESGGGLVQPGGSLRLSCAASGFNIYWYGMHWVRQAPGKGJEWVAQVYPWSGFTYYADSVKGRFTISADTSKNTAYLQMNSLRAEDTAVYYCSRDYYSYSLDYWGQGTLVTVSS

SEQ ID NO: 893 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSWYSPSTFGQGTKVEIKRTGGGSGGGGSGGGASEVQLVESGGGLVQPGGSLRLSCAASGFNISASGMHWVRQAPGKGLEWVAWIWGGSSYTYYADSVKGRFTISADTSKNTAYLQMNSLRAEDTAVYYCSRGQYTSYMDYWGQGTLVTVSS

SEQ ID NO: 894 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYYAPITFGQGTKVEIKRTGGGSGGGGSGGGASEVQLVESGGGLVQPGGSLRLSCAASGFNFSYYGMHWVRQAPGKGLEWVAWIYPFSGYTNYADSVKGRFTISADTSKNTAYLQMNSLRAEDTAVYYCSREYYSRAFDYWGQGTLVTVSS

SEQ ID NO: 895 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYYSPWTFGQGTKVEIKRTGGGSGGGGSGGGASEVQLVESGGGLVQPGGSLRLSCAASGFNISYSNIHWVRQAPGKGLEWVAMIYGTRGGTYYADSVKGRFTISADTSKNTAYLQMNSLRAEDTAVYYSCRYYSYAMDYWGQGTLVTVSS

SEQ ID NO: 896 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQAYYPPWTFGQGTKVEIKRTGGGSGGGGSGGGASEVQLVESGGGLVQPGGSLRLSCAASGFNVSRWAMHWVRQAPGKGLEWVARVYPSGYLTYYADSVKGRFTISADTSKNTAYLQMNSLRAEDTAVYYCSRNMQSYMDYWGQGTLVTVSS

SEQ ID NO: 897 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYSSFPVTFGQGTKVEIKRTGGGSGGGGSGGGASEVQLVESGGGLVQPGGSLRLSCAASGFNFSYGGIHWVRQAPGKGLEWVAMIYPLTGYTNYADSVKGRFTISADTSKNTAYLQMNSLRAEDTAVYYCSRDYYYSVDVWGQGTLVTVSS

SEQ ID NO: 898 - protein, 245 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQTYYYPFTFGQGTKVEIKRTGGGSGGGGSGGGASEVQLVESGGGLVQPGGSLRLSCAASGFNLYAWGMHWVRQAPGKGLEWVALVYGGWGSTSYADSVKGRFTISADTSKNTAYLQMNSLRAEDTAVYYCSRAGSSKMSAGAFDYWGQGTLVTVSS

SEQ ID NO: 899 - protein, 244 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYYPYYPWTFGQGTKVEIKRTGGGSGGGGSGGGASEVQLVESGGGLVQPGGSLRLSCAASGFNVSHSAMHWVRQAPGKGLEWVATVHPDWGNTYYADSVKGRFTISADTSKNTAYLQMNSLRAEDTAVYYSCRWQQYYYSFDYWGQGTLVTVSS

SEQ ID NO: 900 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYDRPITFGQGTKVEIKRTGGGSGGGGSGGGASEVQLVESGGGLVQPGGSLRLSCAASGFNIYYEAMHWVRQAPGKGLWEVAQIYPWNDYTYYADSVKGRFTISADTSKNTAYLQMNSLRAEDTAVYYCSRNYYAATMDYWGQGTLVTVSS

SEQ ID NO: 901 - protein, 249 aa, synthetic construct
DIQMTQSPSSLSASVGFRVTITCRASQDVNTAVAWYQQKPGKAPKLLIYSASFLYSGVPSRFSGSRSGTDFTLTISSLQPEDFATYYCQQSYTSPLTFGQGTKVEIKRTGGGSGGGGSGGGASEVQLVESGGGLVQPGGSLRLSCAASGFNIYYGVMHWVRQAPGKGLEWVAMIYPDSSWTYYADSVKGRFTISADTSKNTAYLQMNSLRAEDTAVYYCSRDQDFHYMNYYLSYALDYWGQGTLVTVSS

SEQ ID NO: 902 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYWYYYPITFGQGTKVEIKRTGGGSGGGGSGGGASEVQLVESGGGLVQPGGSLRLSCAASGFNITSTDMHWVRQAPGKGLEWVAISPGGSYTYYADSVKGRFTISADTSKNTAYLQMNSLRAEDTAVYYCSRSAFTGYFDVWGQGTLVTVSS

SEQ ID NO: 903 - protein, 246 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYYAPITFGQGTKVEIKRTGGGSGGGGSGGGASEVQLVESGGGLVQPGGSLRLSACCSGFNVQWSHMHWVRQAPGKGLEWVARLSPPSGYTNYADSVKGRFTISADTSKNTAYLQMNSLRAEDTAVYYCSRLILSKGGYGWAMDYWGQGTLVTVSS

SEQ ID NO: 904 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYYLYQPITFGQGTKVEIKRTGGGSGGGGSGGGASEVQLVESGGGLVQPGGSLRLSCAASGFNIGMYTMHWVRQAPGKGLEWVALVYPDSGYTNYADSVKGRFTISADTSKNTAYLQMNSLRAEDTAVYYCSRYTWQSMDYWGQGTLVTVSS

SEQ ID NO: 905 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSNYPLTFGQGTKVEIKRTGGGSGGGGSGGGASEVQLVESGGGLVQPGGSLRLSCAASGFNVFYGSMHWVRQAPGKGLEWVAFIGPDSTYTYYADSVKGRFTISADTSKNTAYLQMNSLRAEDTAVYYCSRDLGSAYAMDYWGQGTLVTVSS

SEQ ID NO: 906 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYASDPITFGQGTKVEIKRTGGGSGGGGSGGGASEVQLVESGGGLVQPGGSLRLSCAASHFNLDYGWMHWVRQAPGKGLEWVAWVVPGSDYTDYADSVKGRFTISADTSKNTAYLQMNSLRAEDTAVYYCSRFHYTAFDVWGQGTLVTVSS

SEQ ID NO: 907 - protein, 239 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYDPITFGQGTKVEIKRTGGGSGGGGSGGGASEVQLVESGGGLVQPGGSLRLSCAASGFNFSYSAMHWVRQAPGKGLEWVADVVPDGDWTYYADSVKGRFTISADTSKNTAYLQMNSLRAEDTAVYYCSRGWYALDYWGQGTLVTVSS

SEQ ID NO: 908 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQTITDPVTFGQGTKVEIKRTGGGSGGGGSGGGASEVQLVESGGGLVQPGGSLRLSCAASGFNVDWAWMHWVRQAPGKGLEWVAWVVGGSDYTYYADSVKGRFTISADTSKNTAYLQMNSLRAEDTAVYYCSRSYYYAFDYWGQGTLVTVSS

SEQ ID NO: 909 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQLMYDPITFGQGTKVEIKRTGGGSGGGGSGGGASEVQLVESGGGLVQPGGSLRLSCAASGFNFGTYWMHWVRQAPGKGLEWVAWFLPDYDYTLYADSVKGRFTISADTSKNTAYLQMNSLRAEDTAVYYCSRHGEYAFDYWGQGTLVTVSS

SEQ ID NO: 910 - protein, 189 aa, synthetic construct [sequence omitted]

SEQ ID NO: 911 - protein, 1066 aa, synthetic construct
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEIEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFLGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 912 - protein, 393 aa, synthetic construct
MEEPQSDPSVEPPLSQETFSDLWKLLPENNVLSPLPSQAMDDLMLSPDDIEQWFIEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRIFEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKIEGPDSD

SEQ ID NO: 913 - protein, 17 aa, synthetic construct
SARDRGLVSLPSVEAFF

SEQ ID NO: 914 - protein, 13 aa, synthetic construct
CAGRNFGNEKLTF

SEQ ID NO: 915 - protein, 316 aa, synthetic construct
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARDRGLVSLPSVEAFFGQGTRLTVVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGGSG

SEQ ID NO: 916 - protein, 270 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGRNFGNEKLTFGTGTRLTIIPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 917 - protein, 14 aa, synthetic construct
ASYLSGSIYNEQFF

SEQ ID NO: 918 - protein, 14 aa, synthetic construct
CAVRDQSGANNLFF

SEQ ID NO: 919 - protein, 315 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASYLSGSIYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGGSG

SEQ ID NO: 920 - protein, 274 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDQSGANNLFFGTGTRLTVIPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 921 - protein, 12 aa, synthetic construct
ASSYSTERGTIY

SEQ ID NO: 922 - protein, 12 aa, synthetic construct
AASGGGGADGLT

SEQ ID NO: 923 - protein, 339 aa, synthetic construct
MRSQNDFLESPVPLSSMHRYRRPLRPGAPAMSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSTERGTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 924 - protein, 280 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGGGGADGLTFGKGTHLIIQPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 925 - protein, 11 aa, synthetic construct
ASSLADIYEQY

SEQ ID NO: 926 - protein, 12 aa, synthetic construct
ATDRQSSGDKLT

SEQ ID NO: 927 - protein, 309 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLADIYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 928 - protein, 273 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDRQSSGDKLTFGTGTRLAVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 929 - protein, 12 aa, synthetic construct
CASSARNDEAFF

SEQ ID NO: 930 - protein, 12 aa, synthetic construct
CAPGDNFNKFYF

SEQ ID NO: 931 - protein, 307 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSARNDEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 932 - protein, 267 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAPGDNFNKFYFGSGTKLNVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 933 - protein, 12 aa, synthetic construct
CASSLGDSEQYF

SEQ ID NO: 934 - protein, 14 aa, synthetic construct
CAVKSRAGSYQLTF

SEQ ID NO: 935 - protein, 308 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGDSEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 936 - protein, 274 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKSRAGSYQLTFGKGTKLSVIPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 937 - protein, 14 aa, synthetic construct
CASSQRSNTGELFF

SEQ ID NO: 938 - protein, 16 aa, synthetic construct
CVVSGGGSSNTGKLIF

SEQ ID NO: 939 - protein, 314 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSQRSNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGGSG

SEQ ID NO: 940 - protein, 276 aa, synthetic construct
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVSGGGSSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 941 - protein, 14 aa, synthetic construct
CASGGRDSTDTQYF

SEQ ID NO: 942 - protein, 14 aa, synthetic construct
CATDAGGGADGLTF

SEQ ID NO: 943 - protein, 309 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASGGRDSTDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 944 - protein, 273 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAGGGADGLTFGKGTHLIIQPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 945 - protein, 14 aa, synthetic construct
ASSTSFWEVNTEAF

SEQ ID NO: 946 - protein, 11 aa, synthetic construct
AGGPNTGNQFY

SEQ ID NO: 947 - protein, 313 aa, synthetic construct
MGPGLLCWVLLCLLGAGSVETGVTQSPTALIKTRGQQVTLRCSSQSGHNTVSWYQQALGQGPQFIFQYYREEENGRGNFPPRFSGLQFPNYSSELNVNALELDDSALYLCASSTSFWEVNTEAFFGQGTRLTVVKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKDSRGGGSG

SEQ ID NO: 948 - protein, 267 aa, synthetic construct
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGGPNTGNQFYFGTGTSLTVIPNDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 949 - protein, 12 aa, synthetic construct
ASSKRGWPYEQY

SEQ ID NO: 950 - protein, 15 aa, synthetic construct
AVREEVLYNQGGKLI

SEQ ID NO: 951 - protein, 311 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSKRGWPYEQYFGPGTRLTVTKDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKDSRGGGSG

SEQ ID NO: 952 - protein, 268 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVREEVLYNQGGKLIFGQGTELSVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 953 - protein, 11 aa, synthetic construct
ASSLADIYEQY

SEQ ID NO: 954 - protein, 12 aa, synthetic construct
ATDRQSSGDKLT

SEQ ID NO: 955 - protein, 305 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLADIYEQYFGPGTRLTVTKDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 956 - protein, 269 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDRQSSGDKLTFGTGTRLAVRPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 957 - protein, 11 aa, synthetic construct
ASSSTDRIEAF

SEQ ID NO: 958 - protein, 13 aa, synthetic construct
ALYIYGGSQGNLI

SEQ ID NO: 959 - protein, 15 aa, synthetic construct
ASTTFKTGRAIEKLF

SEQ ID NO: 960 - protein, 9 aa, synthetic construct
ATYNFNKFY

SEQ ID NO: 961 - protein, 13 aa, synthetic construct
ASSSRGHSGTEAF

SEQ ID NO: 962 - protein, 12 aa, synthetic construct
AVRDRGGSYIPT

SEQ ID NO: 963 - protein, 13 aa, synthetic construct
ASSSRGHSGTEAF

SEQ ID NO: 964 - protein, 11 aa, synthetic construct
AGLYSSASKII

SEQ ID NO: 965 - protein, 10 aa, synthetic construct
ATYKVGDEQF

SEQ ID NO: 966 - protein, 10 aa, synthetic construct
LANTGGFKTI

SEQ ID NO: 967 - protein, 14 aa, synthetic construct
ASSDWLAGAKDEQY

SEQ ID NO: 968 - protein, 9 aa, synthetic construct
AETGFQKLV

SEQ ID NO: 969 - protein, 311 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSDWLAGAKDEQYFGPGTRLTVTKDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKDFGGSG

SEQ ID NO: 970 - protein, 266 aa, synthetic construct
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAETGFQKLVFGTGTRLLVSPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 971 - protein, 11 aa, synthetic construct
ASSLVASNEQF

SEQ ID NO: 972 - protein, 10 aa, synthetic construct
ATDPLDYKLS

SEQ ID NO: 973 - protein, 13 aa, synthetic construct
ASSLGLLLYNEQF

SEQ ID NO: 974 - protein, 10 aa, synthetic construct
ACQGGSEKLV

SEQ ID NO: 975 - protein, 12 aa, synthetic construct
ASSLGDSYEQYF

SEQ ID NO: 976 - protein, 12 aa, synthetic construct
ATDAQTGANNLF

SEQ ID NO: 977 - protein, 12 aa, synthetic construct
ASSEWGSTGELF

SEQ ID NO: 978 - protein, 13 aa, synthetic construct
AANAGGTSYGKLT

SEQ ID NO: 979 - protein, 12 aa, synthetic construct
ASSEWGSTGELF

SEQ ID NO: 980 - protein, 10 aa, synthetic construct
AVDIIGGKST

SEQ ID NO: 981 - protein, 11 aa, synthetic construct
ASSEYTMGTQY

SEQ ID NO: 982 - protein, 14 aa, synthetic construct
AASAVGQEYGNKLV

SEQ ID NO: 983 - protein, 322 aa, synthetic construct
MSNTAFPDPAWNTTLLSWVALFLLGTSSANSGVVQSPRYIIKGKGERSILKCIPISGHLSVAWYQQTQGQELKFFIQHYDKMERDKGNLPSRFSVQQFDDYHSEMNMSALELEDSAVYFCASSLTDPLDSDYTFGSGTRLLVIEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKDSRGGGSG

SEQ ID NO: 984 - protein, 269 aa, synthetic construct
MQRNLGAVLGILWVQICWVRGDQVEQSPSALSLHEGTDSALRCNFTTTMRSVQWFRQNSRGSLISLFYLASGTKENGRLKSAFDSKERRYSTLHIRDAQLEDSGTYFCAADSSNTGYQNFYFGKGTSLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 985 - protein, 16 aa, synthetic construct
CASSYGPGQHNSPLHF

SEQ ID NO: 986 - protein, 16 aa, synthetic construct
CALSGPSGAGSYQLTF

SEQ ID NO: 987 - protein, 315 aa, synthetic construct
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYGPGQHNSPLHFGNGTRLTVTEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADCGFTSVSYQQGVLSATILYEILLGKATLYAVLVSALVLMAMVKRKDFGGSG

SEQ ID NO: 988 - protein, 278 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSGPSGAGSYQLTFGKGTKLSVIPNIQNPDPAVYQLRDSKSSDKSVCLFTDFDSQTNVSQSKDSVDYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 989 - protein, 14 aa, synthetic construct
CASSVAGGGQETQY

SEQ ID NO: 990 - protein, 13 aa, synthetic construct
CALSEAGTYKYIF

SEQ ID NO: 991 - protein, 316 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVAGGGQETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGSG

SEQ ID NO: 992 - protein, 275 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 993 - protein, 16 aa, synthetic construct
CASSLSFRQGLREQYF

SEQ ID NO: 994 - protein, 15 aa, synthetic construct
CAVNPPDTGFQKLVF

SEQ ID NO: 995 - protein, 317 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLSFRQGLREQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGSG

SEQ ID NO: 996 - protein, 274 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCAVNPPDTGFQKLVFGTGTRLLVSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 997 - protein, 10 aa, synthetic construct
CASKVYGYTF

SEQ ID NO: 998 - protein, 16 aa, synthetic construct
CLVGDFNSNSGYALNF

SEQ ID NO: 999 - protein, 310 aa, synthetic construct
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASKVYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGGSG

SEQ ID NO: 1000 - protein, 271 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDFNSNSGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 1001 - protein, 19 aa, synthetic construct
CSASPRAGQLSSYNSPLHF

SEQ ID NO: 1002 - protein, 14 aa, synthetic construct
CAVDKDGGYQKVTF

SEQ ID NO: 1003 - protein, 327 aa, synthetic construct
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSASPRAGQLSSYNSPLHFGNGTRLTVTEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGGSG

SEQ ID NO: 1004 - protein, 271 aa, synthetic construct
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDKDGGYQKVTFGTGTKLQVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 1005 - protein, 16 aa, synthetic construct
CASSLYGGSISYEQYF

SEQ ID NO: 1006 - protein, 12 aa, synthetic construct
CATDPGGFKTIF

SEQ ID NO: 1007 - protein, 318 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLYGGSISYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGSG

SEQ ID NO: 1008 - protein, 271 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDPGGFKTIFGAGTRLFVKANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 1009 - protein, 14 aa, synthetic construct
CAISESERYYEQYF

SEQ ID NO: 1010 - protein, 14 aa, synthetic construct
CATFPNFGNEKLTF

SEQ ID NO: 1011 - protein, 315 aa, synthetic construct
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISESERYYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGG

SEQ ID NO: 1012 - protein, 273 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATFPNFGNEKLTFGTGTRLTIIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 1013 - protein, 17 aa, synthetic construct
SARDRGLVSLPSVEAFF

SEQ ID NO: 1014 - protein, 14 aa, synthetic construct
ASYLSGSIYNEQFF

SEQ ID NO: 1015 - protein, 12 aa, synthetic construct
ASSYSTERGTIY

SEQ ID NO: 1016 - protein, 11 aa, synthetic construct
ASSLADIYEQY

SEQ ID NO: 1017 - protein, 12 aa, synthetic construct
CASSARNDEAFF

SEQ ID NO: 1018 - protein, 12 aa, synthetic construct
CASSLGDSEQYF

SEQ ID NO: 1019 - protein, 14 aa, synthetic construct
CASSORSNTGELFF

SEQ ID NO: 1020 - protein, 14 aa, synthetic construct
CASGGRDSTDTQYF

SEQ ID NO: 1021 - protein, 14 aa, synthetic construct
ASSTSFWEVNTEAF

SEQ ID NO: 1022 - protein, 12 aa, synthetic construct
ASSKRGWPYEQY

SEQ ID NO: 1023 - protein, 11 aa, synthetic construct
ASSLADIYEQY

SEQ ID NO: 1024 - protein, 11 aa, synthetic construct
ASSSTDRIEAF

SEQ ID NO: 1025 - protein, 15 aa, synthetic construct
ASTTFKTGRAIEKLF

SEQ ID NO: 1026 - protein, 13 aa, synthetic construct
ASSSRGHSGTEAF

SEQ ID NO: 1027 - protein, 13 aa, synthetic construct
ASSSRGHSGTEAF

SEQ ID NO: 1028 - protein, 10 aa, synthetic construct
ATYKVGDEQF

SEQ ID NO: 1029 - protein, 14 aa, synthetic construct
ASSDWLAGAKDEQY

SEQ ID NO: 1030 - protein, 11 aa, synthetic construct
ASSLVASNEQF

SEQ ID NO: 1031 - protein, 13 aa, synthetic construct
ASSLGLLLYNEQF

SEQ ID NO: 1032 - protein, 12 aa, synthetic construct
ASSLGDSYEQYF

SEQ ID NO: 1033 - protein, 12 aa, synthetic construct
ASSEWGSTGELF

SEQ ID NO: 1034 - protein, 12 aa, synthetic construct
ASSEWGSTGELF

SEQ ID NO: 1035 - protein, 11 aa, synthetic construct
ASSEYTMGTQY

SEQ ID NO: 1036 - protein, 17 aa, synthetic construct
ASSLDFVLAGSYSYNEQ

SEQ ID NO: 1037 - protein, 13 aa, synthetic construct
ASSQSGQGPYEQY

SEQ ID NO: 1038 - protein, 12 aa, synthetic construct
ASSRTAMNTEAF

SEQ ID NO: 1039 - protein, 393 aa, Homo sapiens [sequence omitted]

SEQ ID NO: 1040 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 1041 - protein, 195 aa, synthetic construct
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 1042 - protein, 25 aa, synthetic construct
PILLTISILSFFSVALLVILACVLW

SEQ ID NO: 1043 - protein, 242 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 1044 - protein, 215 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 1045 - protein, 290 aa, synthetic construct
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 1046 - protein, 9 aa, synthetic construct
IVGAIGVGK

SEQ ID NO: 1047 - protein, 9 aa, synthetic construct
IVGAFGTGK

SEQ ID NO: 1048 - protein, 9 aa, synthetic construct
AVGAEGRVK

SEQ ID NO: 1049 - protein, 9 aa, synthetic construct
IVGANGTGK

SEQ ID NO: 1050 - protein, 9 aa, synthetic construct
EVGAKGQLK

SEQ ID NO: 1051 - protein, 9 aa, synthetic construct
VVGAGGVSK

SEQ ID NO: 1052 - protein, 9 aa, synthetic construct
VVGASGVGK

SEQ ID NO: 1053 - protein, 9 aa, synthetic construct
VVGASGVGK

SEQ ID NO: 1054 - protein, 9 aa, synthetic construct
VVGAGGVGK

SEQ ID NO: 1055 - protein, 9 aa, synthetic construct
VVGAGGVGK

SEQ ID NO: 1056 - protein, 9 aa, synthetic construct
VVGAGGVGK

SEQ ID NO: 1057 - protein, 9 aa, synthetic construct
VVGARGVGK

SEQ ID NO: 1058 - protein, 9 aa, synthetic construct
ASGAVGSAK

SEQ ID NO: 1059 - protein, 9 aa, synthetic construct
IVGAIGVGK

SEQ ID NO: 1060 - protein, 9 aa, synthetic construct
NVGSTGVAK

SEQ ID NO: 1061 - protein, 9 aa, synthetic construct
VIGSAGTGK

SEQ ID NO: 1062 - protein, 9 aa, synthetic construct
MQGAAGIGK

SEQ ID NO: 1063 - protein, 9 aa, synthetic construct
ILGATGTGK

SEQ ID NO: 1064 - protein, 9 aa, synthetic construct
IVGCTGSGK

SEQ ID NO: 1065 - protein, 9 aa, synthetic construct
ICGSVGSGK

SEQ ID NO: 1066 - protein, 9 aa, synthetic construct
QMGAAGSGR

SEQ ID NO: 1067 - protein, 9 aa, synthetic construct
PVGAAGSAR

SEQ ID NO: 1068 - protein, 9 aa, synthetic construct
MNGSIGAGR

SEQ ID NO: 1069 - protein, 9 aa, synthetic construct
TCGSCGCGY

SEQ ID NO: 1070 - protein, 44 aa, synthetic construct
ANCQIMPSTVNCQILMSTVGACSACQHIMTVGACISTVAGMRKY

SEQ ID NO: 1071 - protein, 9 aa, synthetic construct
NVGAGGCAK